(19) United States
(12) Patent Application Publication
(10) Pub. No.: US 2022/0289717 A1
(43) Pub. Date: Sep. 15, 2022

(54) COMPOUND, MIXTURE THEREOF, MATERIAL FOR ORGANIC ELECTROLUMINESCENCE DEVICE, ORGANIC ELECTROLUMINESCENCE DEVICE, AND ELECTRONIC DEVICE

(71) Applicant: IDEMITSU KOSAN CO.,LTD., Tokyo (JP)

(72) Inventors: Masato MITANI, Sodegaura-shi (JP); Masatoshi SAITO, Sodegaura-shi (JP); Kei YOSHIDA, Sodegaura-shi (JP); Tomokatsu KUSHIDA, Sodegaura-shi (JP); Sayaka MIZUTANI, Sodegaura-shi (JP)

(73) Assignee: IDEMITSU KOSAN CO.,LTD., Tokyo (JP)

(21) Appl. No.: 17/573,406

(22) Filed: Jan. 11, 2022

(30) Foreign Application Priority Data
Jan. 15, 2021 (JP) 2021-005152

(51) Int. Cl. C07D 403/10; H01L 51/00; C07D 405/14; C07D 409/14; C07D 403/14; C07D 401/14; C07D 401/10; C07D 519/00

(52) U.S. Cl. CPC C07D 403/10; H01L 51/508; C07D 405/14; C07D 409/14; H01L 51/0074; H01L 51/0073; H01L 51/0072; C07D 403/14; C07D 401/14; H01L 51/0069; H01L 51/0067; H01L 51/0062; C07D 401/10; H01L 51/0071; C07D 519/00; H01L 51/0055

(57) ABSTRACT

Provided is a compound represented by a formula (1). In the formula (1): X¹ to X⁵ each independently represent a nitrogen atom or CR¹⁰; two or more of X¹ to X⁵ are nitrogen atoms; Y¹ to Y⁵ each independently represent a nitrogen atom or CR²⁰; one or more of Y¹ to Y⁵ are nitrogen atoms; R¹⁰, R²⁰, and R⁵ to R⁷ forming neither a monocyclic ring nor a fused ring, and R⁸ and R⁹ each independently represent a hydrogen atom, an aryl group, a heterocyclic group, or the like; a represents 0, 1, 2, or 3; b represents 0, 1, 2, or 3; and L1 and L2 each independently represent a single bond, an arylene group, a divalent heterocyclic group, or the like.

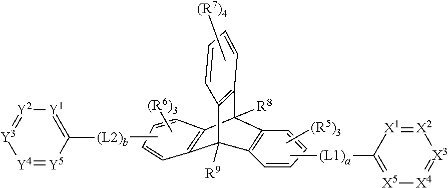

The entire disclosure of Japanese Patent Application No. 2021-005152, filed Jan. 15, 2021 is expressly incorporated by reference herein.

### TECHNICAL FIELD

The present invention relates to a compound, a mixture thereof, a material for organic electroluminescence device, an organic electroluminescence device, and an electronic device.

### BACKGROUND ART

Upon a voltage being applied to an organic electroluminescence device (hereinafter, may be referred to as “organic EL device”), holes are injected from an anode to an emitting layer, while electrons are injected from a cathode to the emitting layer. The injected holes and electrons recombine with each other in the emitting layer to form excitons. Specifically, singlet and triplet excitons are formed at proportions of 25%:75%, respectively, due to the electron spin statistics theorem.

Organic EL devices have been applied to full-color displays included in cellular mobile phones, televisions, and the like.

There have been various studies of compounds included in organic EL devices in order to enhance the performance of the organic EL devices (e.g., see Document 1: International Publication No. WO 2019/163959, Document 2: International Publication No. WO 2018/173882, Document 3: International Publication No. WO 99/19419, Document 4: U.S. Patent Application Publication No. 2007/051944, Document 5: KR 10-2006-0122874 A, and Document 6: Japanese Unexamined Patent Application Publication No. 2007-520875).

Examples of the performance of an organic EL device include luminance, emission wavelength, chromaticity, luminous efficiency, drive voltage, and lifetime.

### SUMMARY OF THE INVENTION

An object of the invention is to provide a compound capable of enhancing the performance of an organic EL device, a mixture thereof capable of enhancing the performance of an organic EL device, a material for organic electroluminescence device including the compound or mixture, an organic electroluminescence device including the compound or mixture, and an electronic device including the organic electroluminescence device.

According to an aspect of the invention, there is provided a compound represented by a formula (1) below.

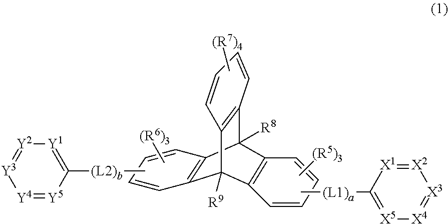

In the formula (1),

X¹ to X⁵ each independently represent a nitrogen atom or CR¹⁰,

two or more of X¹ to X⁵ are nitrogen atoms, and

at least one combination of adjacent two or more of a plurality of R¹⁰ are bonded to each other to form a substituted or unsubstituted monocyclic ring, are bonded to each other to form a substituted or unsubstituted fused ring, or are not bonded to each other,

Y¹ to Y⁵ each independently represent a nitrogen atom or CR²⁰ ,

one or more of Y¹ to Y⁵ are nitrogen atoms,

at least one combination of adjacent two or more of a plurality of R²⁰ are bonded to each other to form a substituted or unsubstituted monocyclic ring, are bonded to each other to form a substituted or unsubstituted fused ring, or are not bonded to each other,

at least one combination of adjacent two or more of a plurality of R⁵ are bonded to each other to form a substituted or unsubstituted monocyclic ring, are bonded to each other to form a substituted or unsubstituted fused ring, or are not bonded to each other,

at least one combination of adjacent two or more of a plurality of R⁶ are bonded to each other to form a substituted or unsubstituted monocyclic ring, are bonded to each other to form a substituted or unsubstituted fused ring, or are not bonded to each other,

at least one combination of adjacent two or more of a plurality of R⁷ are bonded to each other to form a substituted or unsubstituted monocyclic ring, are bonded to each other to form a substituted or unsubstituted fused ring, or are not bonded to each other,

R¹⁰, R²⁰, and R⁵ to R⁷ forming neither the substituted or unsubstituted monocyclic ring nor the substituted or unsubstituted fused ring, and R⁸ and R⁹ each independently represent a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms,

when a plurality of R¹⁰ are present, the plurality of R¹⁰ are mutually the same or different,

when a plurality of R²⁰ are present, the plurality of R²⁰ are mutually the same or different,

the plurality of R⁵ are mutually the same or different,

the plurality of R⁶ are mutually the same or different,

the plurality of R⁷ are mutually the same or different,

a represents 0, 1, 2, or 3,

when a is 2 or 3, a plurality of L1 are mutually the same or different,

b represents 0, 1, 2, or 3,

when b is 2 or 3, a plurality of L2 are mutually the same or different,

when a and b are each independently 1, 2, or 3,

L1 and L2 each independently represent a single bond, a substituted or unsubstituted arylene group having 6 to 50 ring carbon atoms, or a substituted or unsubstituted divalent heterocyclic group having 5 to 50 ring atoms,

when only one of Y¹ to Y⁵ is a nitrogen atom, L2 is not a single bond, or any of Y¹ to Y⁵ other than a nitrogen atom is not CH.

In the compound represented by the formula (1),

R₉₀₁ to R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,

when two or more R₉₀₁ are present, the two or more R₉₀₁ are mutually the same or different; when two or more R₉₀₂ are present, the two or more R₉₀₂ are mutually the same or different; when two or more R₉₀₃ are present, the two or more R₉₀₃ are mutually the same or different; when two or more R₉₀₄ are present, the two or more R₉₀₄ are mutually the same or different; when two or more R₉₀₅ are present, the two or more R₉₀₅ are mutually the same or different; when two or more R₉₀₆ are present, the two or more R₉₀₆ are mutually the same or different; and when two or more R₉₀₇ are present, the two or more R₉₀₇ are mutually the same or different.

According to an aspect of the invention, there is provided a mixture including the compound according to the above aspect of the invention as a first compound; and a second compound that is an enantiomer of the first compound.

According to an aspect of the invention, there is provided a material for organic electroluminescence device including the compound according to the above aspect of the invention.

According to an aspect of the invention, there is provided a material for organic electroluminescence device including the mixture according to the above aspect of the invention.

According to an aspect of the invention, there is provided an organic electroluminescence device including: a cathode; an anode; and one or more organic layers interposed between the cathode and the anode, in which at least one of the organic layers includes the compound according to the above aspect of the invention as a first compound.

According to an aspect of the invention, there is provided an electronic device including the organic electroluminescence device according to the above aspect of the invention.

According to an aspect of the invention, there can be provided a compound capable of enhancing the performance of an organic EL device, a mixture thereof capable of enhancing the performance of an organic EL device, a material for organic electroluminescence device including the compound or mixture, an organic electroluminescence device including the compound or mixture, and an electronic device including the organic electroluminescence device.

### BRIEF DESCRIPTION OF DRAWINGS

FIG. 1 is a schematic diagram of an exemplary layer arrangement of an organic EL device according to an aspect of the invention.

FIG. 2 is a schematic diagram of another exemplary layer arrangement of an organic EL device according to an aspect of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium or tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and do not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) below. (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

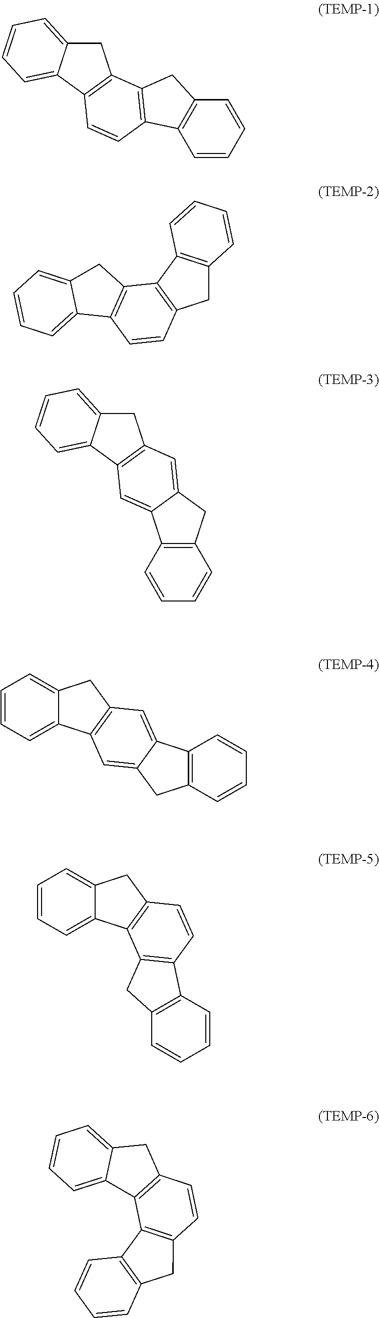

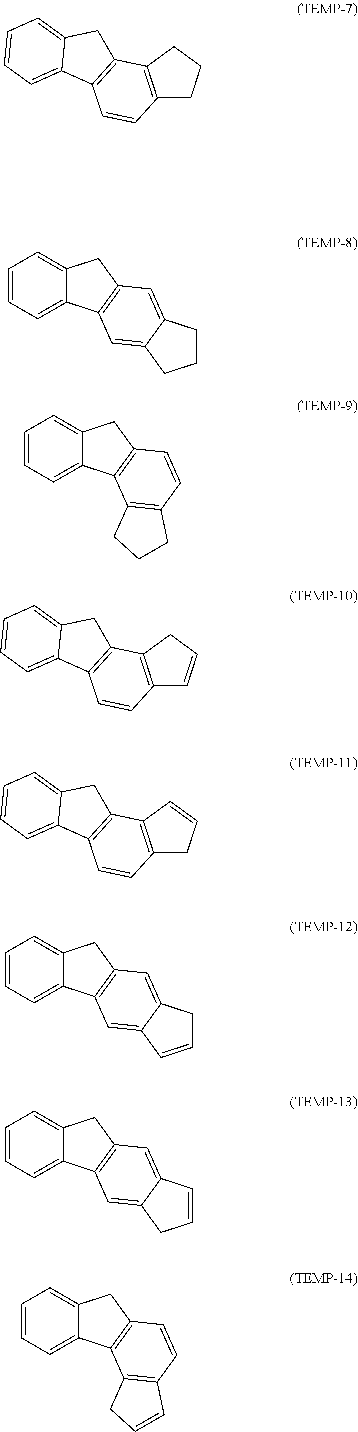

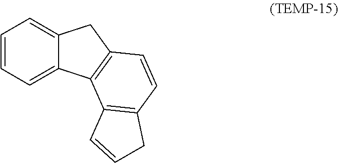 [SMILES: C1=Cc2c(ccc3c2-c2ccccc2C3)C1]

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by formulae (TEMP-16) to (TEMP-33) (Specific Example Group G2A4):

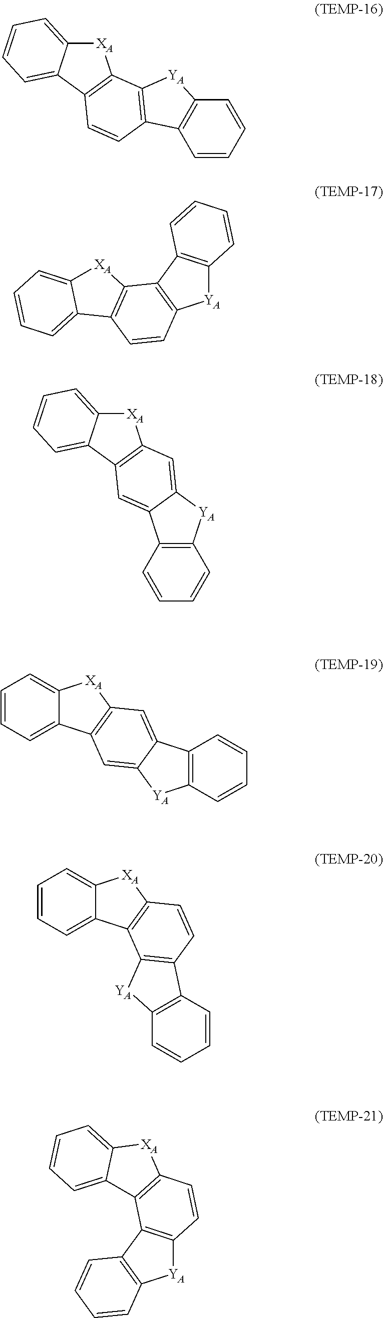

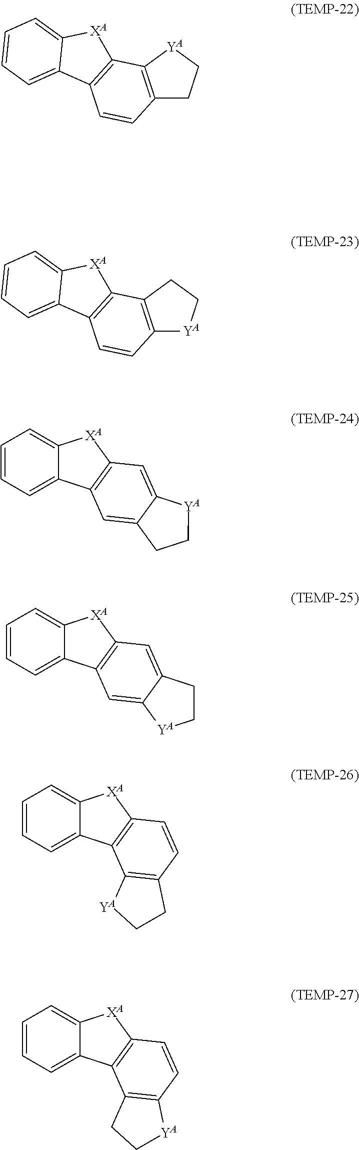

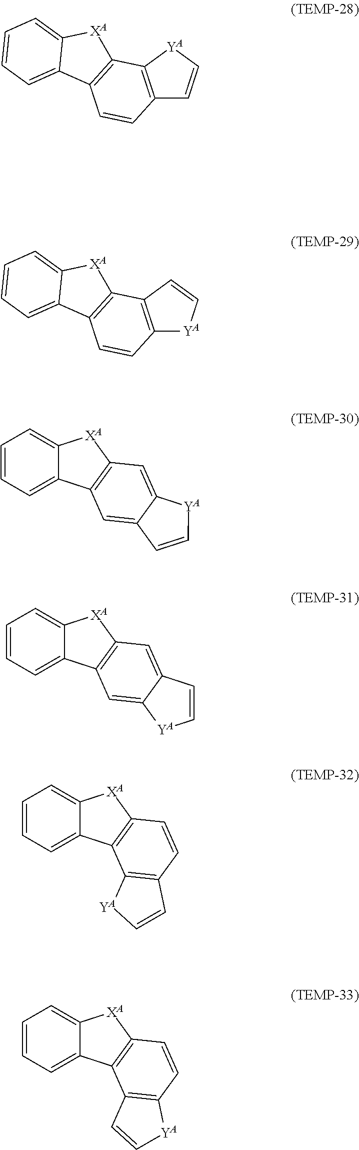

In the formulae (TEMP-16) to (TEMP-33), X_{A} and Y_{A} are each independently an oxygen atom, a sulfur atom, NH, or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group,

t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of XA or YA in a form of NH, and a hydrogen atom of one of XA and YA in a form of a methylene group (CH2).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group,

isobutyl group, s-butyl group, and t-butyl group.

### Substituted Alkyl Group (Specific Example Group G3B):

heptafluoropropyl group (including isomer thereof), pentafluoroethyl group,

2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallylgroup.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent. Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6),

where

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —Si(G1)(G1)(G1) are mutually the same or different;

a plurality of G2 in —Si(G1)(G2)(G2) are mutually the same or different;

a plurality of G1 in —Si(G1)(G1)(G2) are mutually the same or different;

a plurality of G2 in —Si(G2)(G2)(G2) are mutually the same or different;

a plurality of G3 in —Si(G3)(G3)(G3) are mutually the same or different; and

a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —N(G1)(G1) are mutually the same or different;

a plurality of G2 in —N(G2)(G2) are mutually the same or different;

a plurality of G3 in —N(G3)(G3) are mutually the same or different; and

a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, a-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

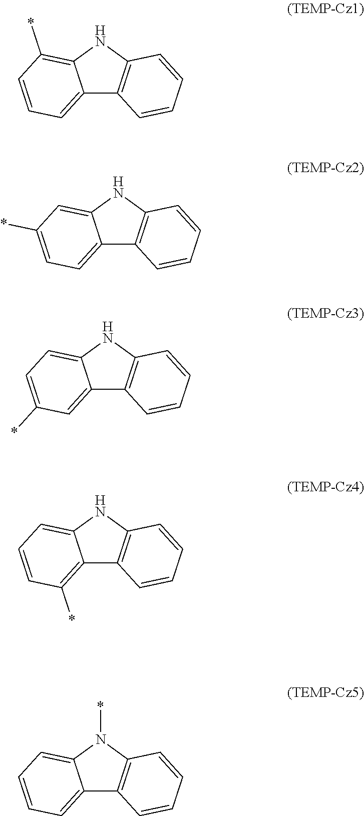 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

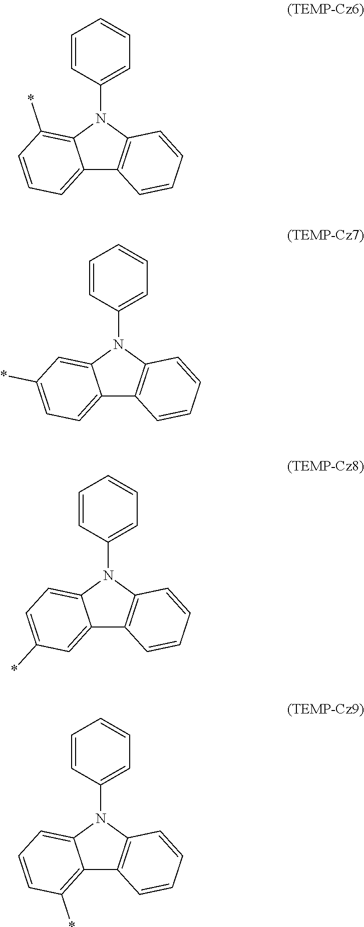 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

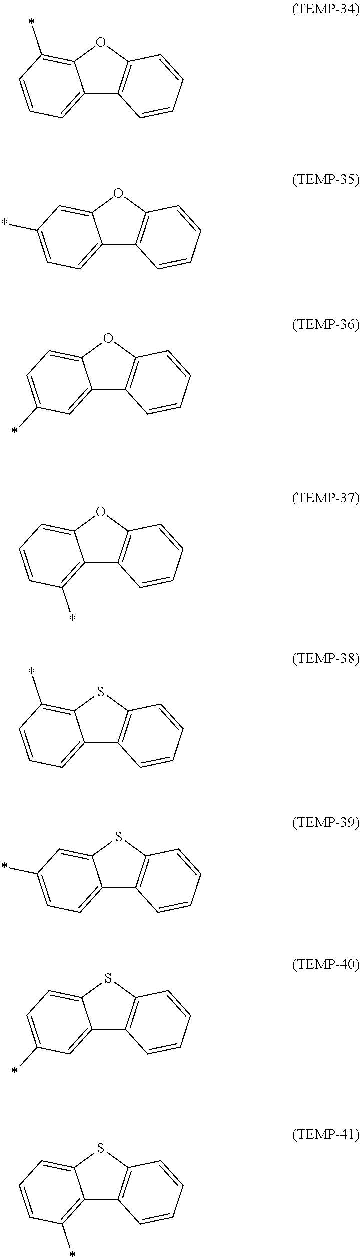

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

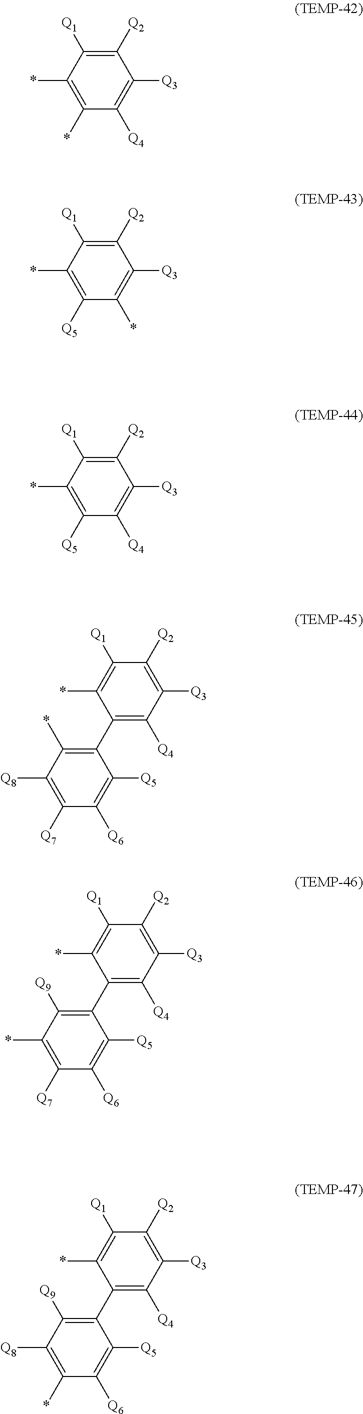

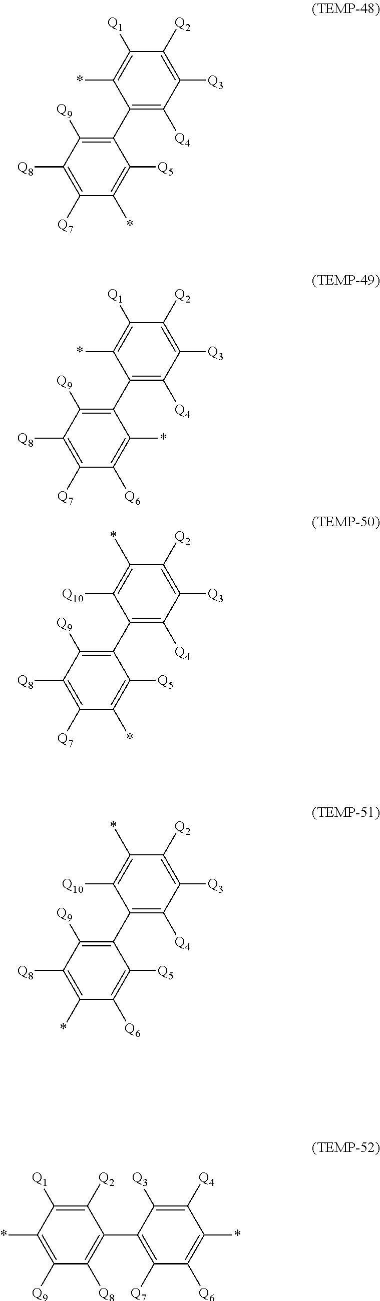

In the formulae (TEMP-42) to (TEMP-52), Q₁ to Q₁₀ are each independently a hydrogen atom or a substituent.

In the formulae (TEMP-42) to (TEMP-52), * represents a bonding position.

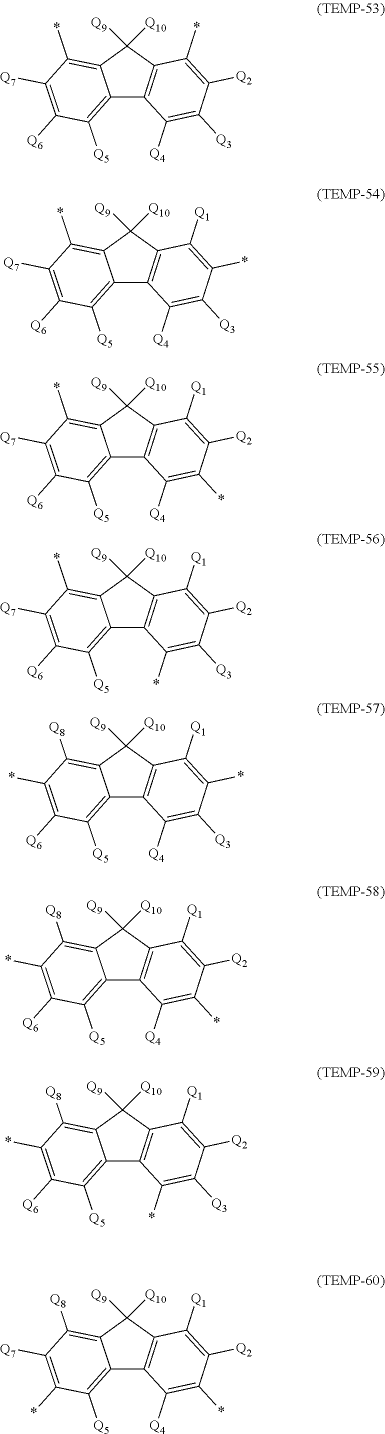

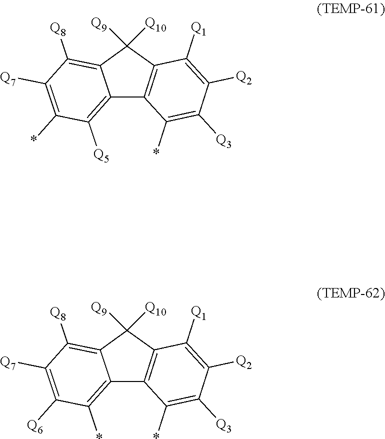

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

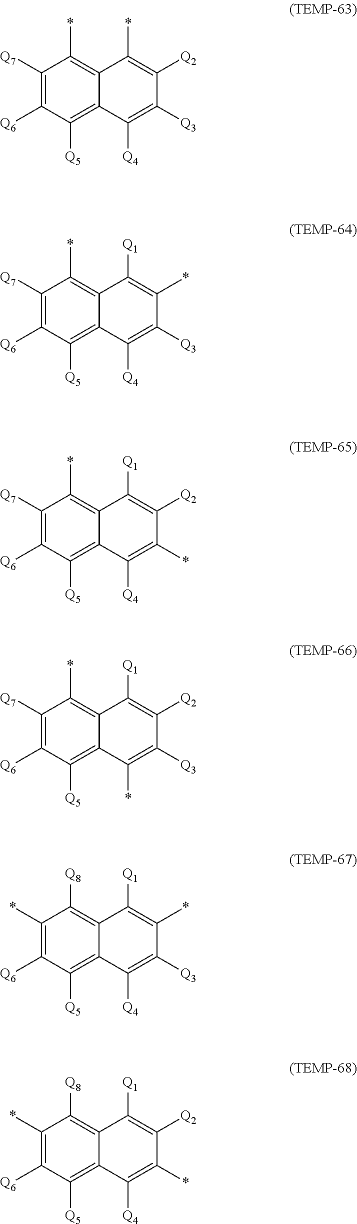

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

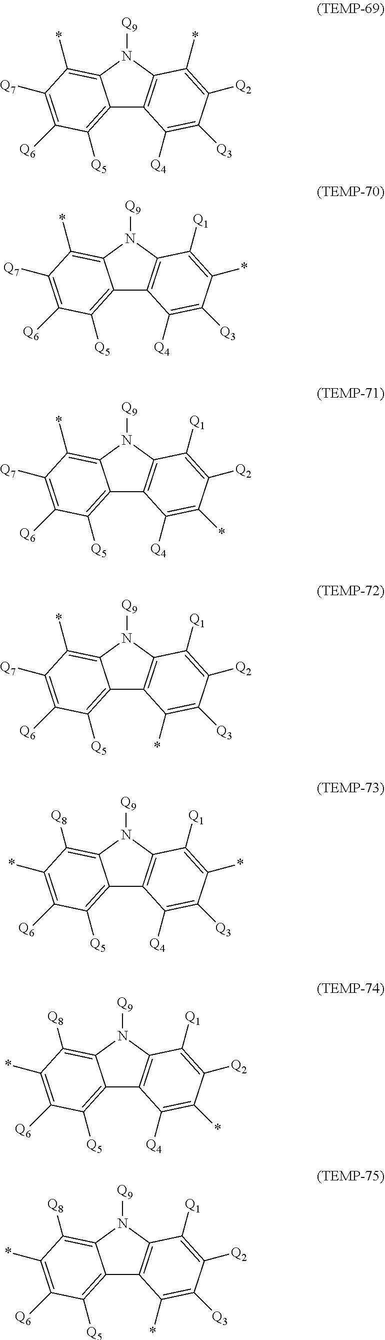

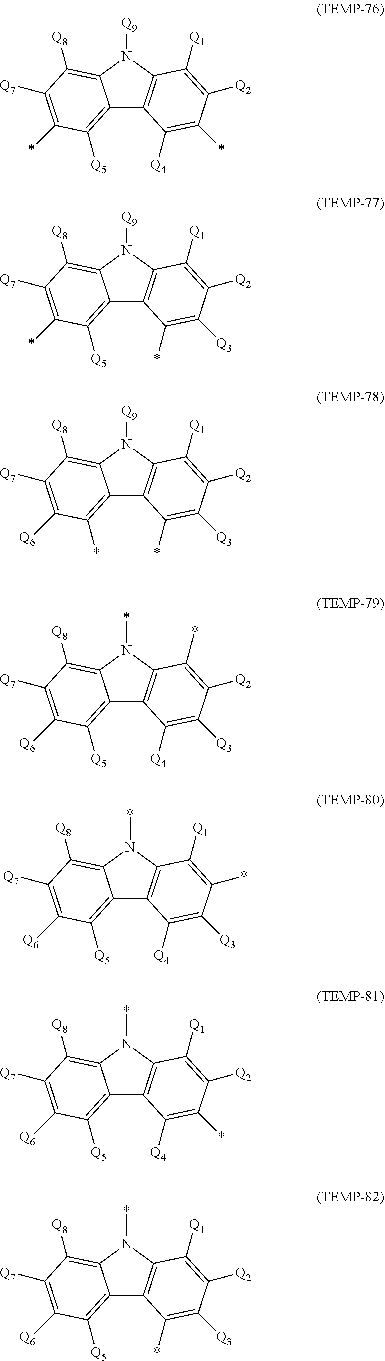

In the formulae (TEMP-69) to (TEMP-82), Q₁ to Q₉ are each independently a hydrogen atom or a substituent.

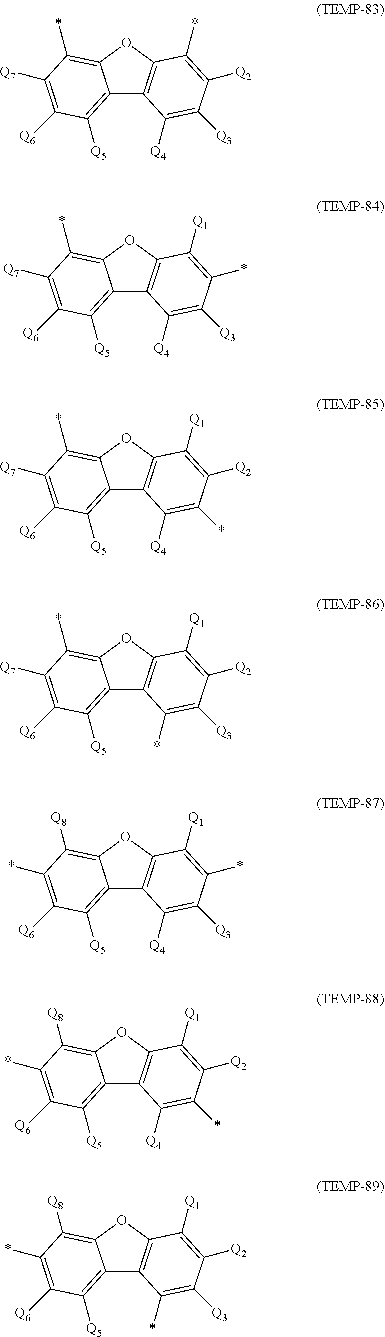

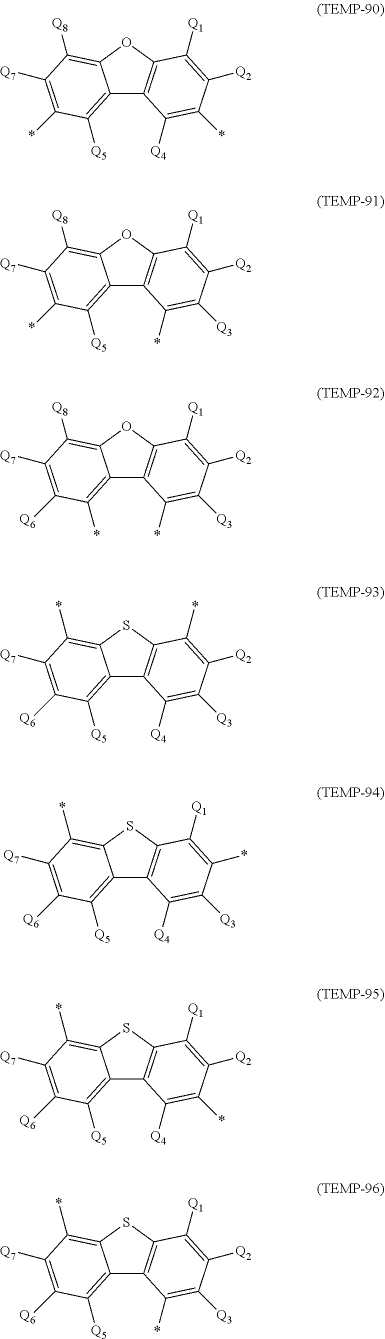

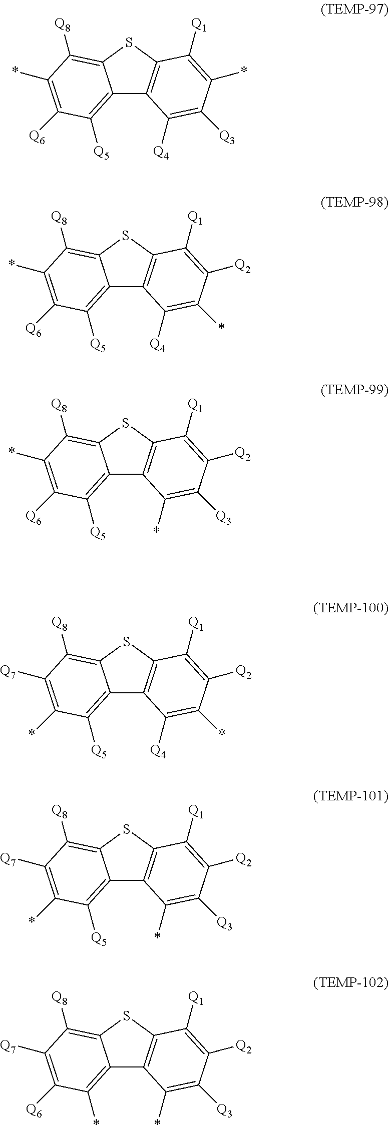

In the formulae (TEMP-83) to (TEMP-102), Q₁ to Q₈ are each independently a hydrogen atom or a substituent.

The substituent mentioned herein has been described above. Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

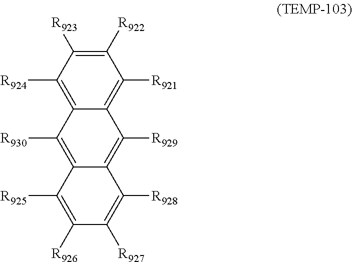

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₀ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring QA and R₉₂₅ and R₉₂₆ are simultaneously mutually bonded to form a ring Q_{B}, the anthracene compound represented by the formula (TEMP-103) is represented by a formula (TEMP-104) below.

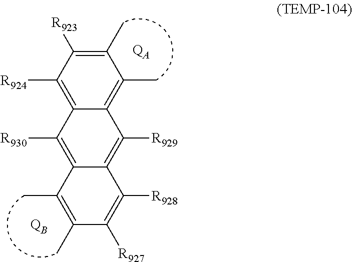

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring QA and R₉₂₂ and R₉₂₃ are mutually bonded to form a ring Qc, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring QA and the ring Qc share R₉₂₂.

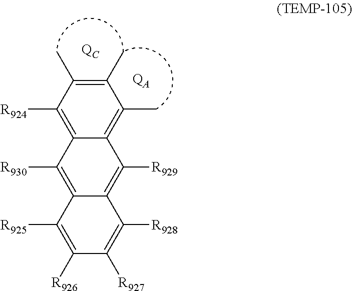

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Qc formed in the formula (TEMP-105) are each a “fused ring.” The ring Q_{A} and the ring Qc in the formula (TEMP-105) are fused to form a fused ring. When the ring Q_{A} in the formula (TMEP-104) is a benzene ring, the ring Q_{A} is a monocyclic ring. When the ring Q_{A} in the formula (TMEP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms,

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,

when two or more R₉₀₁ are present, the two or more R₉₀₁ are mutually the same or different,

when two or more R₉₀₂ are present, the two or more R₉₀₂ are mutually the same or different,

when two or more R₉₀₃ are present, the two or more R₉₀₃ are mutually the same or different,

when two or more R₉₀₄ are present, the two or more R₉₀₄ are mutually the same or different,

when two or more R₉₀₅ are present, the two or more R₉₀₅ are mutually the same or different,

when two or more R₉₀₆ are present, the two or more R₉₀₆ are mutually the same or different, and

when two or more R₉₀₇ are present, the two or more R₉₀₇ are mutually the same or different.

In an exemplary embodiment, the substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Compound

A compound according to a first exemplary embodiment is a compound represented by a formula (1) below.

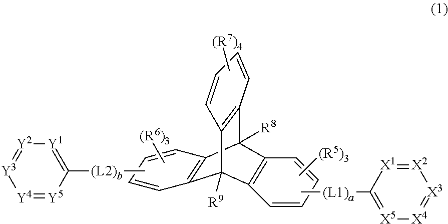

In the formula (1),

X¹ to X⁵ each independently represent a nitrogen atom or CR¹⁰,

two or more of X¹ to X⁵ are nitrogen atoms,

at least one combination of adjacent two or more of a plurality of R¹⁰ are bonded to each other to form a substituted or unsubstituted monocyclic ring, are bonded to each other to form a substituted or unsubstituted fused ring, or are not bonded to each other,

Y¹ to Y⁵ each independently represent a nitrogen atom or CR²⁰,

one or more of Y¹ to Y⁵ are nitrogen atoms,

at least one combination of adjacent two or more of a plurality of R²⁰ are bonded to each other to form a substituted or unsubstituted monocyclic ring, are bonded to each other to form a substituted or unsubstituted fused ring, or are not bonded to each other,

at least one combination of adjacent two or more of a plurality of R⁵ are bonded to each other to form a substituted or unsubstituted monocyclic ring, are bonded to each other to form a substituted or unsubstituted fused ring, or are not bonded to each other,

at least one combination of adjacent two or more of a plurality of R⁶ are bonded to each other to form a substituted or unsubstituted monocyclic ring, are bonded to each other to form a substituted or unsubstituted fused ring, or are not bonded to each other,

at least one combination of adjacent two or more of a plurality of R⁷ are bonded to each other to form a substituted or unsubstituted monocyclic ring, are bonded to each other to form a substituted or unsubstituted fused ring, or are not bonded to each other;

R¹⁰, R²⁰. and R⁵ to R⁷ forming neither the substituted or unsubstituted monocyclic ring nor the substituted or unsubstituted fused ring, and R⁸ and R⁹ each independently represent a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms,

when a plurality of R¹⁰ are present, the plurality of R¹⁰ are mutually the same or different,

when a plurality of R²⁰ are present, the plurality of R²⁰ are mutually the same or different,

the plurality of R⁵ are mutually the same or different,

the plurality of R⁶ are mutually the same or different,

the plurality of R⁷ are mutually the same or different,

a represents 0, 1, 2, or 3,

when a is 2 or 3, a plurality of L1 are mutually the same or different,

b represents 0, 1, 2, or 3,

when b is 2 or 3, a plurality of L2 are mutually the same or different,

when a and b are each independently 1, 2, or 3, L1 and L2 each independently represent a single bond, a substituted or unsubstituted arylene group having 6 to 50 ring carbon atoms, or a substituted or unsubstituted divalent heterocyclic group having 5 to 50 ring atoms, and

when only one of Y¹ to Y⁵ is a nitrogen atom, L2 is not a single bond, or any of Y¹ to Y⁵ other than a nitrogen atom is not CH.

In the compound represented by the formula (1), R₉₀₁ to R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, and

when two or more R₉₀₁ are present, the two or more R₉₀₁ are mutually the same or different; when two or more R₉₀₂ are present, the two or more R₉₀₂ are mutually the same or different; when two or more R₉₀₃ are present, the two or more R₉₀₃ are mutually the same or different; when two or more R₉₀₄ are present, the two or more R₉₀₄ are mutually the same or different; when two or more R₉₀₅ are present, the two or more R₉₀₅ are mutually the same or different; when two or more R₉₀₆ are present, the two or more R₉₀₆ are mutually the same or different; and when two or more R₉₀₇ are present, the two or more R₉₀₇ are mutually the same or different.

A compound in which one substituent including azine is introduced to a triptycene skeleton is known in the related art (e.g., see Documents 1 and 2).

The inventors found that introducing at least one substituent including an azine ring to both terminals of a triptycene skeleton as in the compound represented by the formula (1) results in a compound capable of enhancing the performance of an organic EL device, specifically, a compound capable of reducing the drive voltage of an organic EL device.

The compound according to the exemplary embodiment is suitably used as a material for electron transporting zone of organic EL device (preferably, as a material for electron transporting layer). When the compound according to the exemplary embodiment is used as a material for electron transporting zone, the injectability of electrons to adjacent layers can be improved and, consequently, the reduction in the voltage of an organic EL device may be further readily achieved.

In the compound according to the exemplary embodiment, it is preferable that at least one combination of adjacent two or more of a plurality of R¹⁰ be not bonded to each other, at least one combination of adjacent two or more of a plurality of R²⁰ be not bonded to each other, at least one combination of adjacent two or more of a plurality of R⁵ be not bonded to each other, at least one combination of adjacent two or more of a plurality of R⁶ be not bonded to each other, and at least one combination of adjacent two or more of a plurality of R⁷ be not bonded to each other.

In the compound according to the exemplary embodiment, it is preferable that a combination of adjacent two or more of the plurality of R¹⁰ form neither a substituted or unsubstituted monocyclic ring nor a substituted or unsubstituted fused ring.

In the compound according to the exemplary embodiment, it is preferable that a combination of adjacent two or more of the plurality of R²⁰ form neither a substituted or unsubstituted monocyclic ring nor a substituted or unsubstituted fused ring.

In the compound according to the exemplary embodiment, it is preferable that a combination of adjacent two or more of the plurality of R⁵ form neither a substituted or unsubstituted monocyclic ring nor a substituted or unsubstituted fused ring.

In the compound according to the exemplary embodiment, it is preferable that a combination of adjacent two or more of the plurality of R⁶ form neither a substituted or unsubstituted monocyclic ring nor a substituted or unsubstituted fused ring.

In the compound according to the exemplary embodiment, it is preferable that a combination of adjacent two or more of the plurality of R⁷ form neither a substituted or unsubstituted monocyclic ring nor a substituted or unsubstituted fused ring.

In the formula (1), among partial structures represented by formulae (1A), (1B), and (1C) below, the partial structures represented by the formulae (1A) and (1B) are preferably the same as each other.

In the formula (1), among the partial structures represented by the formulae (1A), (1B), and (1C), the partial structures represented by the formulae (1A) and (1B) are also preferably different from each other.

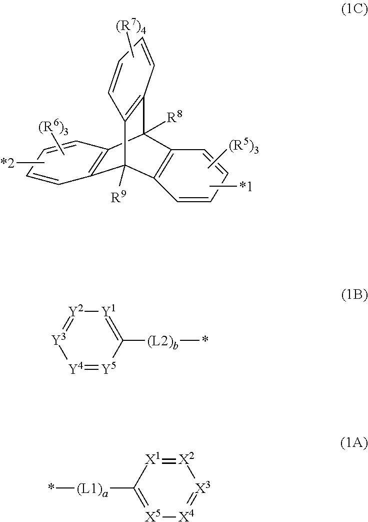

In the formula (1C), R⁵ to R⁹ each independently represent the same as R⁵ to R⁹ in the formula (1),

in the formula (1A), X¹ to X⁵, L1, and a each independently represent the same as X¹ to X⁵, L1, and a in the formula (1), and * represents a bonding position to *1 in the partial structure represented by the formula (1C) in the formula (1), and

in the formula (1B), Y¹ to Y⁵, L2, and b each independently represent the same as Y¹ to Y⁵, L2, and b in the formula (1), and * represents a bonding position to *2 in the partial structure represented by the formula (1C) in the formula (1).

In the formula (1A), two or three of X¹ to X⁵ are preferably nitrogen atoms, and

in the formula (1B), one, two, or three of Y¹ to Y⁵ are preferably nitrogen atoms.

In the compound according to the exemplary embodiment, it is preferable that the partial structure represented by the formula (1A) be represented by any of formulae (1A-1) to (1A-3) below and the partial structure represented by the formula (1B) be represented by any of formulae (1B-1) to (1B-6) below.

In the compound according to the exemplary embodiment, it is more preferable that the partial structure represented by the formula (1A) be represented by a formula (1A-1) or (1A-2) below and the partial structure represented by the formula (1B) be represented by a formula (1B-1), (1B-2), or (1B-3) below.

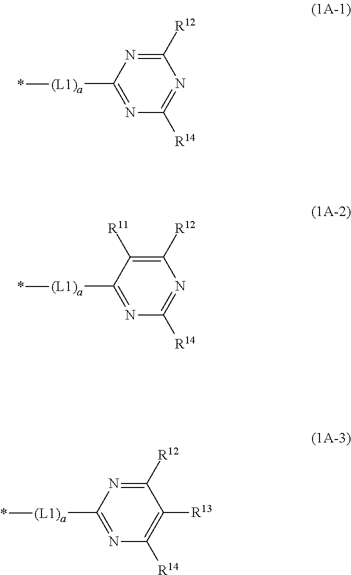

In the formulae (1A-1) to (1A-3), L1 and a each independently represent the same as L1 and a in the formula (1); and R¹¹, R¹², R¹³, and R¹⁴ each independently represent the same as R¹⁰ in the formula (1).

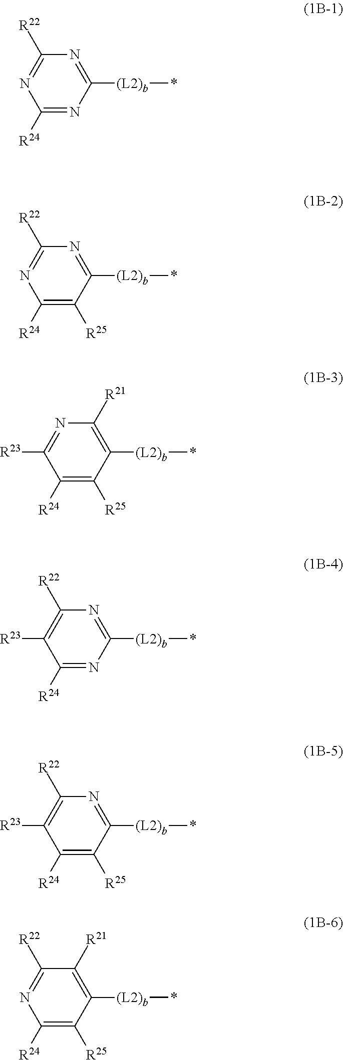

In the formulae (1B-1) to (1B-6), L2 and b each independently represent the same as L2 and b in the formula (1); and R²¹ to R²⁵ each independently represent the same as R²⁰ in the formula (1).

In the compound according to the exemplary embodiment,

when the partial structure represented by the formula (1A) is represented by the formula (1A-1), the partial structure represented by the formula (1B) is preferably represented by the formula (1B-1),

when the partial structure represented by the formula (1A) is represented by the formula (1A-2), the partial structure represented by the formula (1B) is preferably represented by the formula (1B-2); and

when the partial structure represented by the formula (1A) is represented by the formula (1A-3), the partial structure represented by the formula (1B) is preferably represented by the formula (1B-4).

Embodiments of the compound according to the exemplary embodiment in which the partial structure represented by the formula (1A) is represented by the formula (1A-1) and the partial structure represented by the formula (1B) is represented by the formula (1B-1) include an embodiment where the partial structures and the substituents thereof are completely the same and an embodiment where the partial structures are not completely the same.

The embodiment where the partial structures and the substituents thereof are completely the same is an embodiment where, in the formulae (1A-1) and (1B-1), R¹² and R²² are mutually the same, R¹⁴ and R²⁴ are mutually the same, L1 and L2 are mutually the same, and a and b are mutually the same. The embodiment where the partial structures are not completely the same is an embodiment where, in the formulae (1A-1) and (1B-1), at least one of the pairs of R¹² and R²², R¹⁴ and R²⁴, L1 and L2, and a and b are mutually different. In the formulae (1A-1) and (1B-1), “R¹² and R²²” may translate to “R¹² and R²⁴”, and “R¹⁴ and R²⁴” may translate to “R¹⁴ and R²²”.

Similarly, in an embodiment where the partial structure represented by the formula (1A) is represented by the formula (1A-2) and the partial structure represented by the formula (1B) is represented by the formula (1B-2) and in an embodiment where the partial structure represented by the formula (1A) is represented by the formula (1A-3) and the partial structure represented by the formula (1B) is represented by the formula (1B-4), the partial structures and the substituents thereof may be completely the same and may not be completely the same. In the formulae (1A-3) and (1B-4), “R¹² and R²²” may translate to “R¹² and R²⁴” and “R¹⁴ and R²⁴” may translate to “R¹⁴ and R²²”.

In the compound according to the exemplary embodiment, the partial structure represented by the formula (1A) and the partial structure represented by the formula (1B) and the substituents thereof may be completely the same and may not be completely the same.

In the compound according to the exemplary embodiment, the compound represented by the formula (1) is preferably a compound represented by a formula (10) below.

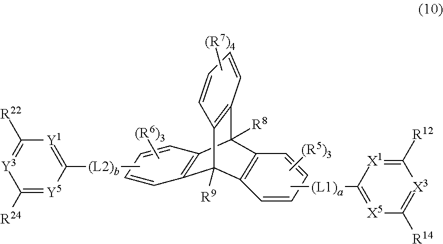

In the formula (10), X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and

R¹² and R¹⁴ each independently represent the same as R¹⁰ in the formula (1); and R²² and R²⁴ each independently represent the same as R²⁰ in the formula (1).

In the compound according to the exemplary embodiment, the compound represented by the formula (1) is preferably a compound represented by a formula (10-1a) below or an enantiomer of the compound represented by the formula (10-1a).

Enantiomers are a pair of stereoisomers that are non-superimposable mirror images.

The enantiomer of the compound represented by the formula (10-1a) can be represented by a formula (10-1b) below.

Herein, the structure of one of a pair of enantiomers may be described as a representative.

When the compound according to the exemplary embodiment is used, only one of the enantiomers may be used alone and the other enantiomer may be also used alone. Or, as described in a second exemplary embodiment below, a mixture including one of the enantiomers (first compound) and the other enantiomer (second compound) may be used.

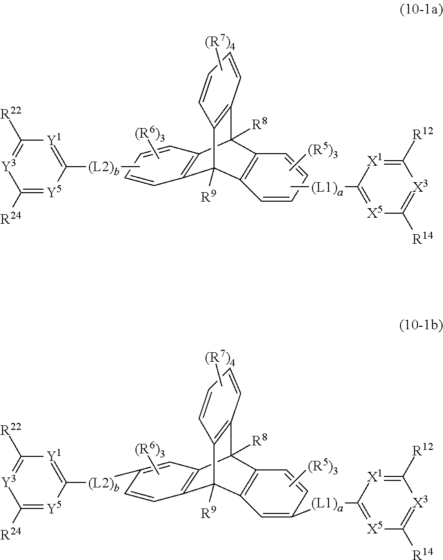

In the formulae (10-1a) and (10-1b),

X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and

R¹² and R¹⁴ each independently represent the same as R¹⁰ in the formula (1); and R²² and R²⁴ each independently represent the same as R²⁰ in the formula (1).

In the compound according to the exemplary embodiment, the compound represented by the formula (1) is also preferably a compound represented by a formula (10-2a) below or an enantiomer of the compound represented by the formula (10-2a).

The enantiomer of the compound represented by the formula (10-2a) can be represented by, for example, a formula (10-2b) below.

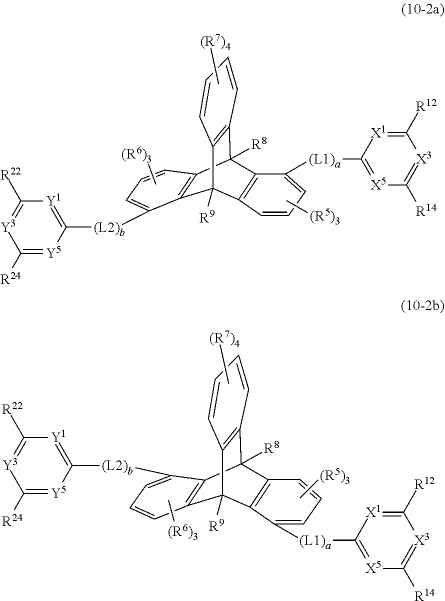

In the formulae (10-2a) and (10-2b),

X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and

R¹² and R¹⁴ each independently represent the same as R¹⁰ in the formula (1); and R²² and R²⁴ each independently represent the same as R²⁰ in the formula (1).

In the compound according to the exemplary embodiment, the compound represented by the formula (1) is also preferably a compound represented by a formula (10-3a) below or an enantiomer of the compound represented by the formula (10-3a).

The enantiomer of the compound represented by the formula (10-3a) can be represented by, for example, a formula (10-3b) below.

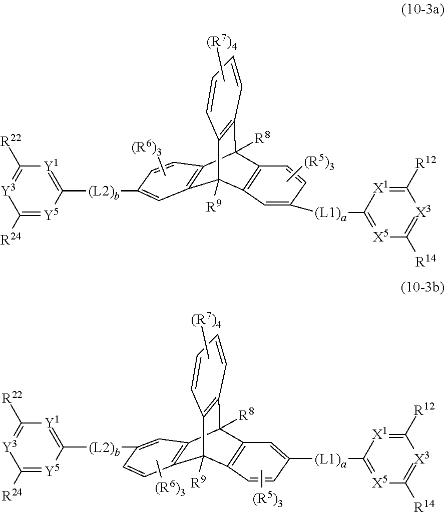

In the formulae (10-3a) and (10-3b),

X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and

R¹² and R¹⁴ each independently represent the same as R¹⁰ in the formula (1); and R²² and R²⁴ each independently represent the same as R²⁰ in the formula (1).

In the compound according to the exemplary embodiment, it is preferable that L1 and L2 each independently represent a single bond, a substituted or unsubstituted arylene group having 6 to 30 ring carbon atoms, or a substituted or unsubstituted divalent heterocyclic group having 5 to 30 ring atoms.

In the compound according to the exemplary embodiment, it is preferable that L1 and L2 each independently represent a single bond, a substituted or unsubstituted phenylene group, a substituted or unsubstituted biphenylene group, a substituted or unsubstituted terphenylene group, a substituted or unsubstituted naphthylene group, a substituted or unsubstituted phenanthrylene group, a substituted or unsubstituted fluorenylene group, a substituted or unsubstituted dibenzofuranylene group, a substituted or unsubstituted dibenzothienylene group, a substituted or unsubstituted pyridylene group, or a substituted or unsubstituted quinolylene group.

In the compound according to the exemplary embodiment, a is preferably 0 or 1.

In the compound according to the exemplary embodiment, b is preferably 0 or 1.

In the compound according to the exemplary embodiment, it is also preferable that L1 and L2 each independently represent at least one group selected from the group consisting of groups represented by formulae (L-1) to (L-16) below.

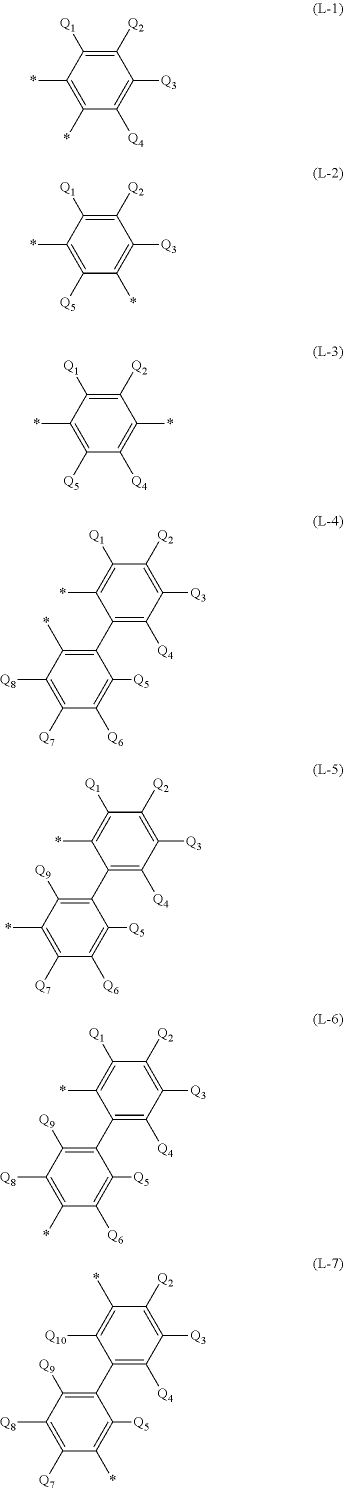

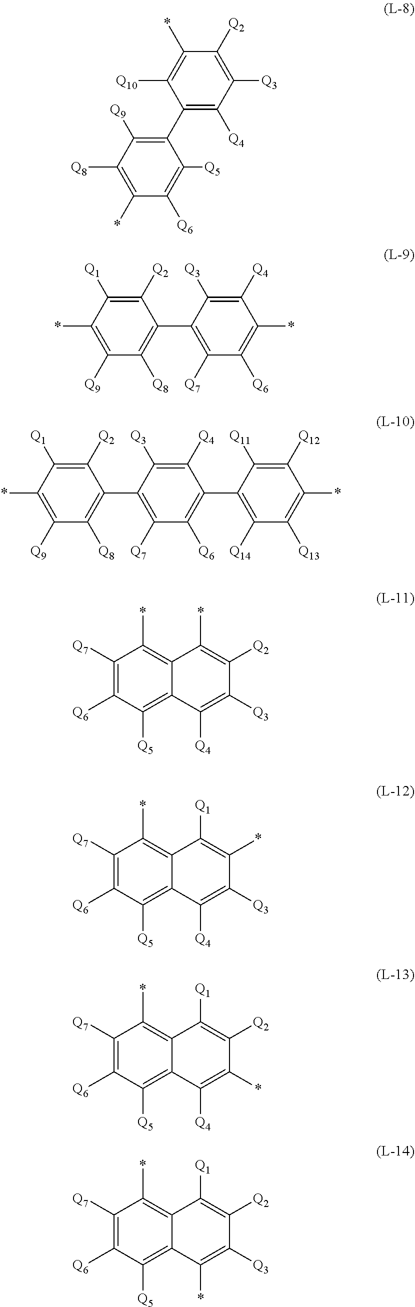

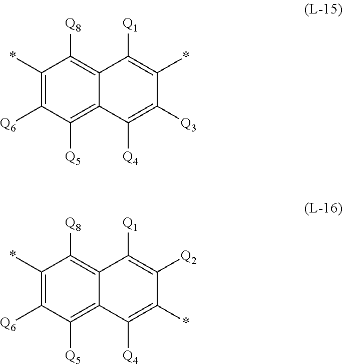

In the formulae (L-1) to (L-16), Q₁ to Q₁₄ preferably each independently represent a hydrogen atom, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms, and more preferably each independently represent a hydrogen atom, an unsubstituted alkyl group having 1 to 18 carbon atoms, an unsubstituted cycloalkyl group having 3 to 18 ring carbon atoms, an unsubstituted aryl group having 6 to 18 ring carbon atoms, or an unsubstituted heterocyclic group having 5 to 18 ring atoms.

In the compound according to the exemplary embodiment, R¹⁰, R²⁰, and R⁵ to R⁹ preferably each independently represent a hydrogen atom, a halogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —N(R₉₀₆)(R₉₀₇), a substituted or unsubstituted aryl group having 6 to 30 ring carbon atoms, or a substituted or unsubstituted heterocyclic group having 5 to 30 ring atoms.

In the compound according to the exemplary embodiment, R¹⁰, R²⁰, and R⁵ to R⁹ preferably each independently represent a hydrogen atom, a halogen atom, a cyano group, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms.

In the compound according to the exemplary embodiment, R¹⁰, R²⁰, and R⁵ to R⁹ preferably each independently represent a hydrogen atom or a group represented by any of formulae (A1) to (A31) below.

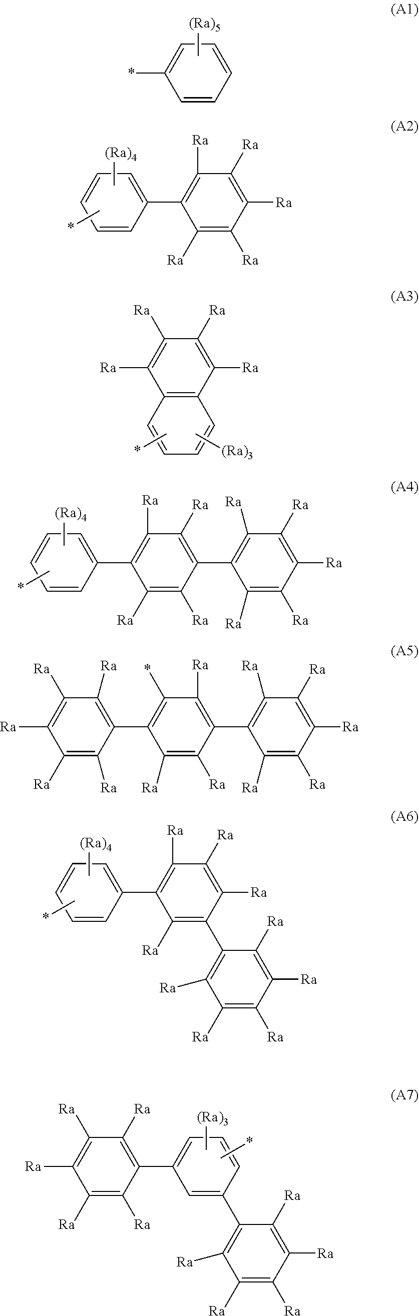

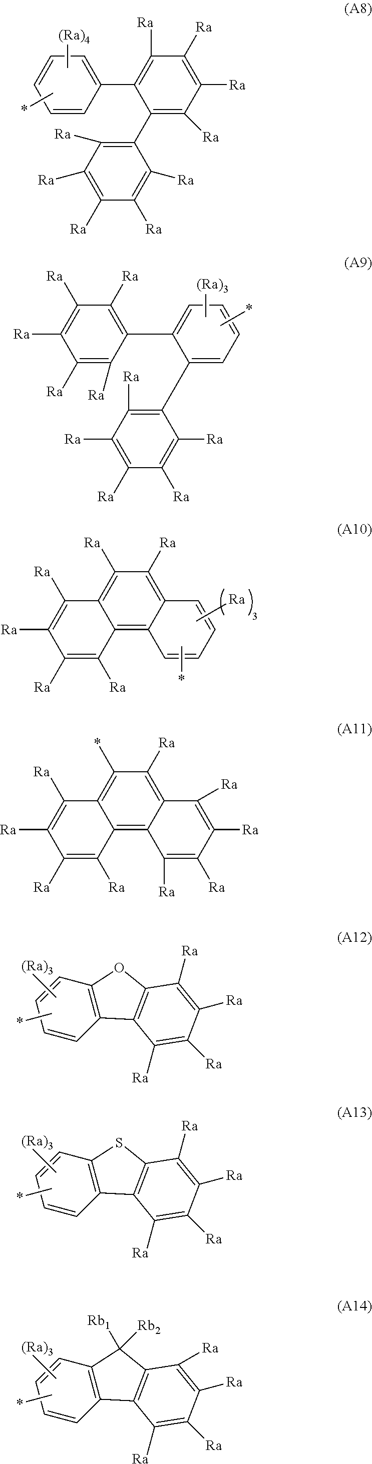

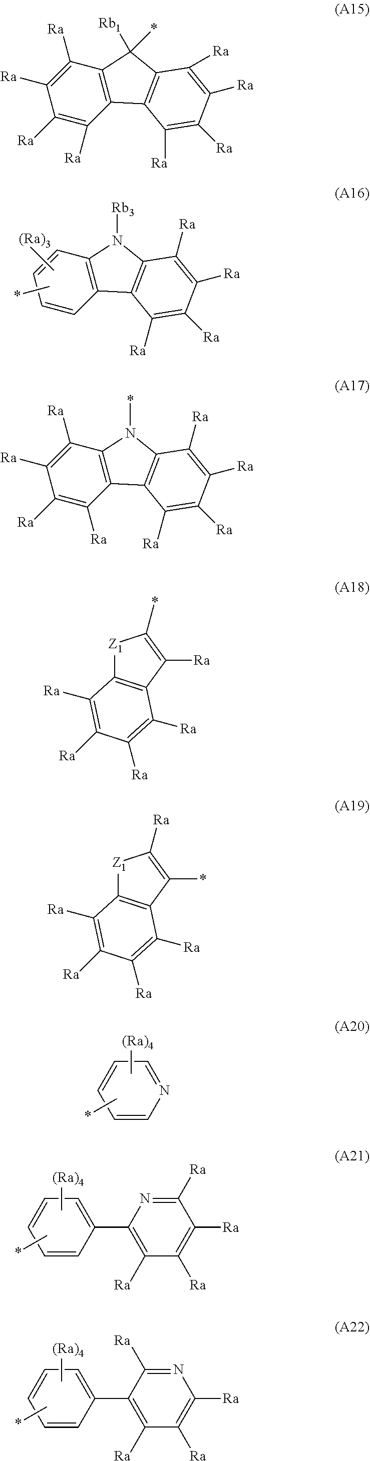

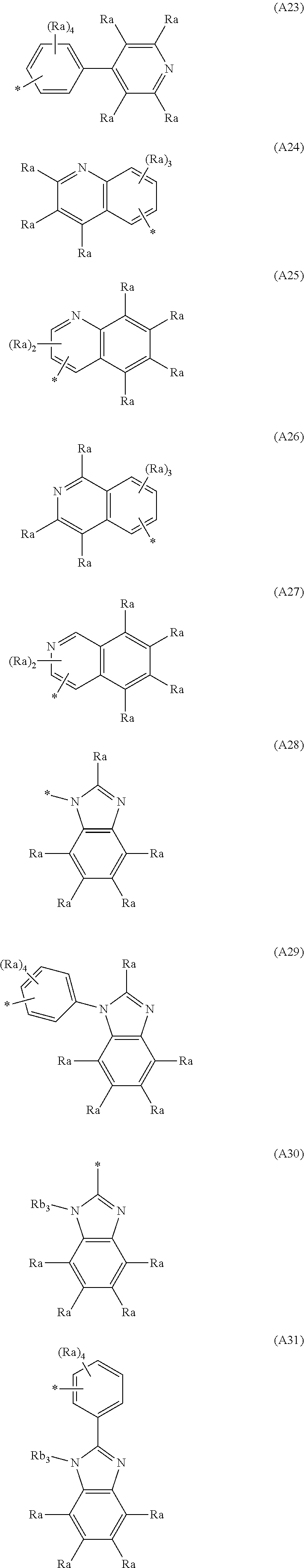

In the formulae (A1) to (A31),

Z₁ represents an oxygen atom, a sulfur atom, or NRb₃;

at least one combination of adjacent two or more of a plurality of Ra are bonded to each other to form a substituted or unsubstituted monocyclic ring, are bonded to each other to form a substituted or unsubstituted fused ring, or are not bonded to each other;

a pair of Rb₁ and Rb₂ are bonded to each other to form a substituted or unsubstituted monocyclic ring, are bonded to each other to form a substituted or unsubstituted fused ring, or are not bonded to each other;

Rb₁ and Rb₂ forming neither the substituted or unsubstituted monocyclic ring nor the substituted or unsubstituted fused ring and Rb₃ each independently represent a hydrogen atom, a substituted or unsubstituted alkyl group having 1 to 30 carbon atoms, a substituted or unsubstituted cycloalkyl group having 3 to 30 ring carbon atoms, a substituted or unsubstituted aryl group having 6 to 30 ring carbon atoms, or a substituted or unsubstituted heterocyclic group having 5 to 30 ring atoms,

Ra forming neither the substituted or unsubstituted monocyclic ring nor the substituted or unsubstituted fused ring each independently represent a hydrogen atom, a halogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —N(R₉₀₆)(R₉₀₇), a substituted or unsubstituted aryl group having 6 to 30 ring carbon atoms, or a substituted or unsubstituted heterocyclic group having 5 to 30 ring atoms,

R₉₀₁ to R₉₀₄, R₉₀₆, and R₉₀₇ each independently represent the same as R₉₀₁ to R₉₀₄, R₉₀₆, and R₉₀₇ in the formula (1),

a plurality of Ra are mutually the same or different,

when a plurality of Z₁ are present, the plurality of Z₁ are mutually the same or different,

when a plurality of Rb₁ are present, the plurality of Rb₁ are mutually the same or different,

when a plurality of Rb₂ are present, the plurality of Rb₂ are mutually the same or different, and

when a plurality of Rb₃ are present, the plurality of Rb₃ are mutually the same or different.

It is also preferable that, in the formulae (A1) to (A31), a combination of adjacent two or more of the plurality of Ra form neither a substituted or unsubstituted monocyclic ring nor a substituted or unsubstituted fused ring.

It is also preferable that, in the formula (A14), Rb₁ and Rb₂ be bonded to each other to form a substituted or unsubstituted monocyclic ring or be bonded to each other to form a substituted or unsubstituted fused ring.

In the formulae (A1) to (A31), Rb₁, Rb₂, Rb₃, and Ra preferably each independently represent a hydrogen atom, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms, and more preferably each independently represent a hydrogen atom, an unsubstituted alkyl group having 1 to 18 carbon atoms, an unsubstituted cycloalkyl group having 3 to 18 ring carbon atoms, an unsubstituted aryl group having 6 to 18 ring carbon atoms, or an unsubstituted heterocyclic group having 5 to 18 ring atoms.

In the compound according to the exemplary embodiment, R⁵ to R⁹ preferably represent a hydrogen atom, and R¹⁰ and R²⁰ preferably each independently represent a hydrogen atom, a halogen atom, a cyano group, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms.

In the compound according to the exemplary embodiment, R₉₀₁ to R₉₀₇ preferably each independently represent a hydrogen atom, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms, and more preferably each independently represent a hydrogen atom, an unsubstituted alkyl group having 1 to 18 carbon atoms, an unsubstituted cycloalkyl group having 3 to 18 ring carbon atoms, an unsubstituted aryl group having 6 to 18 ring carbon atoms, or an unsubstituted heterocyclic group having 5 to 18 ring atoms.

### Manufacturing Method of Compound According to Exemplary Embodiment

The compound according to the exemplary embodiment can be manufactured according to, for instance, a method described later in Examples. The compound of the exemplary embodiment can be manufactured, for instance, by application of known substitution reactions and/or materials tailored for a target compound according to reactions described later in Examples.

Specific examples of the compound according to the exemplary embodiment include the following compounds. Note that the invention is not limited to the specific examples of the compound.

When the compound according to the exemplary embodiment is an enantiomer, only one of the structures is illustrated as a representative.

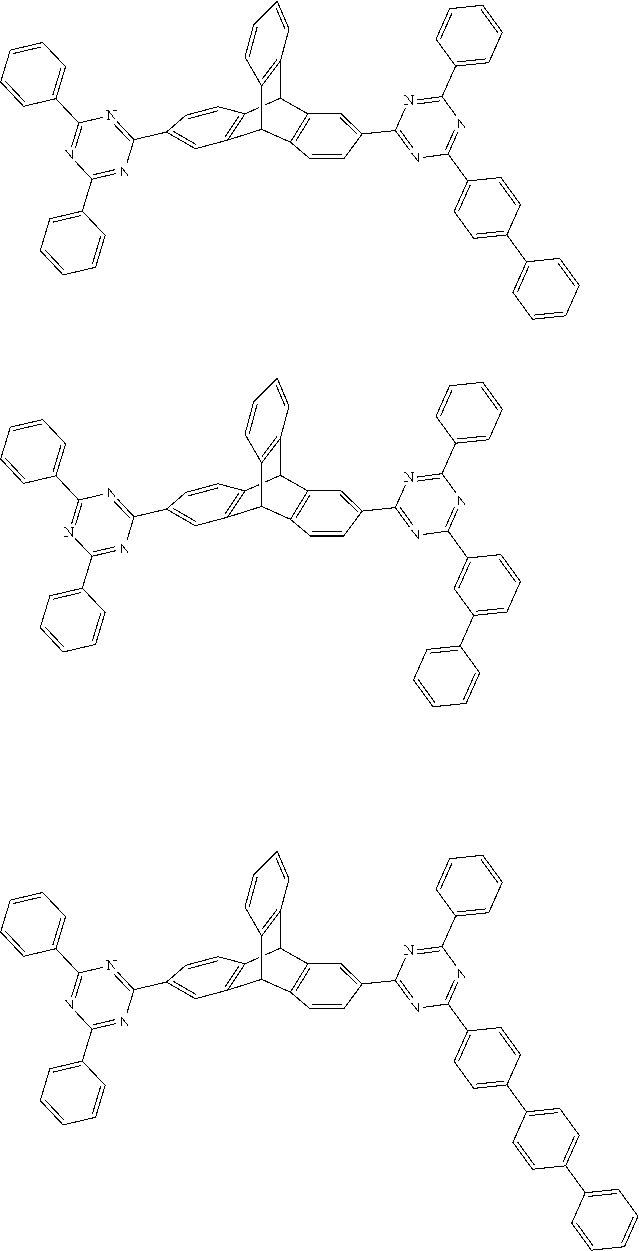 [SMILES: c1ccc(-c2ccc(-c3nc(-c4ccccc4)nc(-c4ccc5c(c4)C4c6ccccc6C5c5cc(-c6nc(-c7ccccc7)nc(-c7ccccc7)n6)ccc54)n3)cc2)cc1 ; c1ccc(-c2cccc(-c3nc(-c4ccccc4)nc(-c4ccc5c(c4)C4c6ccccc6C5c5cc(-c6nc(-c7ccccc7)nc(-c7ccccc7)n6)ccc54)n3)c2)cc1 ; c1ccc(-c2ccc(-c3ccc(-c4nc(-c5ccccc5)nc(-c5ccc6c(c5)C5c7ccccc7C6c6cc(-c7nc(-c8ccccc8)nc(-c8ccccc8)n7)ccc65)n4)cc3)cc2)cc1]

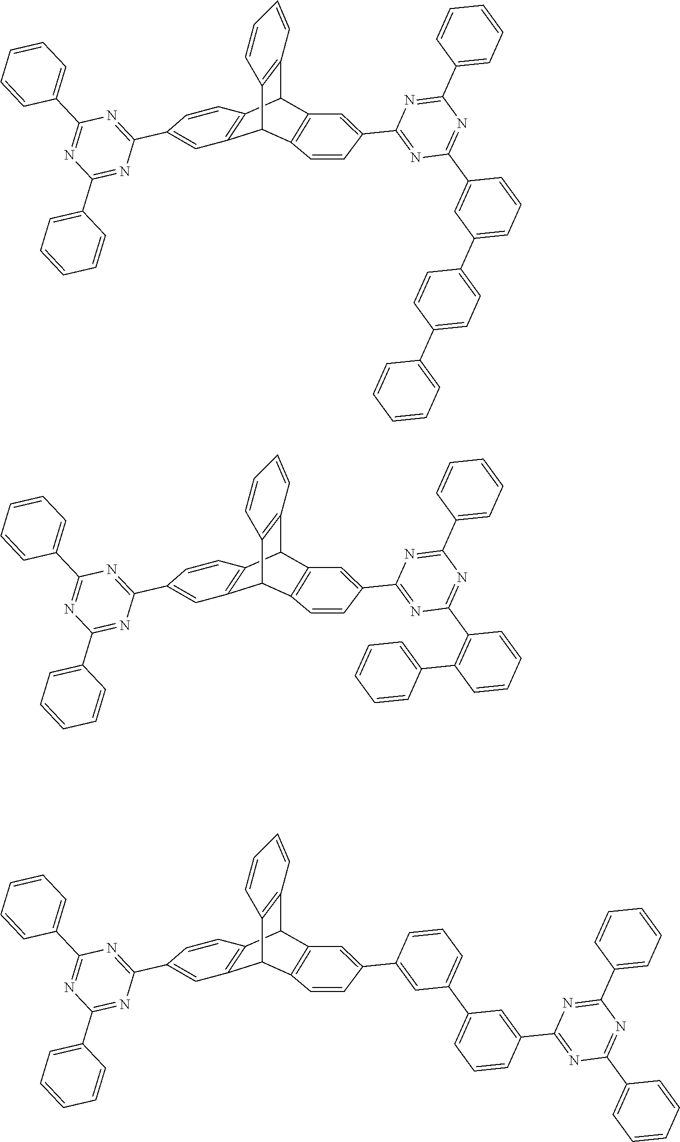 [SMILES: c1ccc(-c2ccc(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)cc2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccccc6)n5)ccc43)n2)cc1 ; c1ccc(-c2nc(-c3ccccc3)nc(-c3cccc(-c4cccc(-c5ccc6c(c5)C5c7ccccc7C6c6cc(-c7nc(-c8ccccc8)nc(-c8ccccc8)n7)ccc65)c4)c3)n2)cc1]

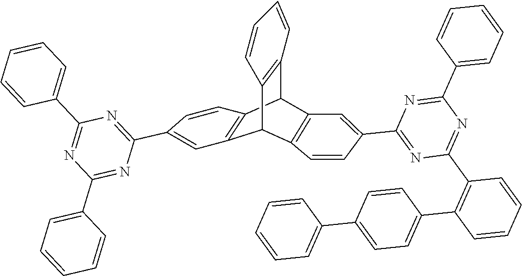 [SMILES: c1ccc(-c2ccc(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)cc2)cc1]

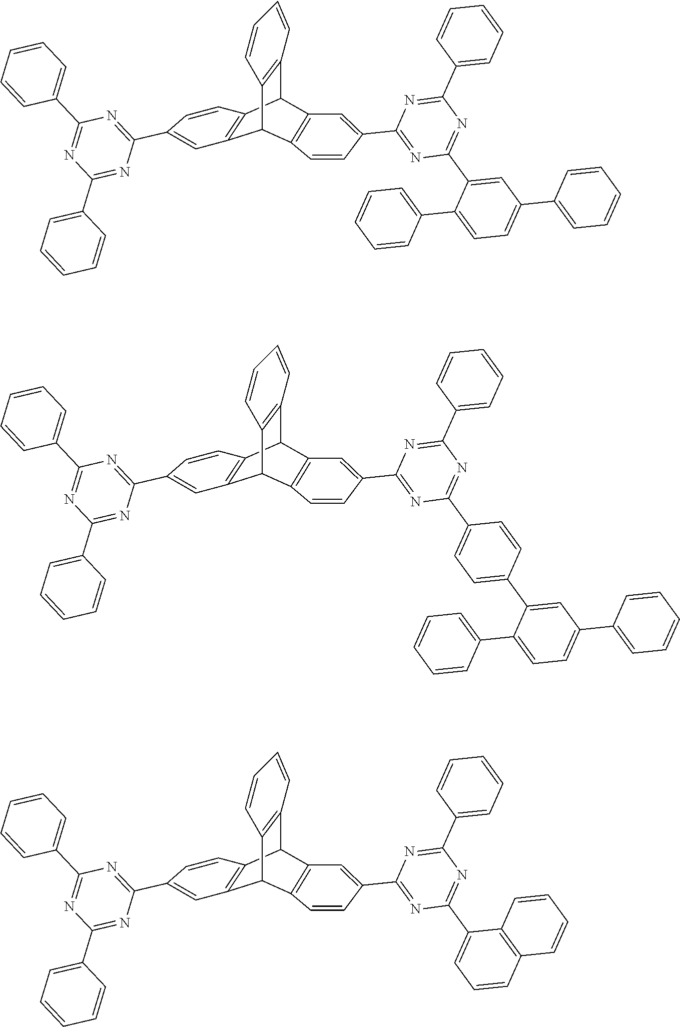 [SMILES: c1ccc(-c2ccc(-c3ccccc3)c(-c3nc(-c4ccccc4)nc(-c4ccc5c(c4)C4c6ccccc6C5c5cc(-c6nc(-c7ccccc7)nc(-c7ccccc7)n6)ccc54)n3)c2)cc1 ; c1ccc(-c2ccc(-c3ccccc3)c(-c3ccc(-c4nc(-c5ccccc5)nc(-c5ccc6c(c5)C5c7ccccc7C6c6cc(-c7nc(-c8ccccc8)nc(-c8ccccc8)n7)ccc65)n4)cc3)c2)cc1 ; c1ccc(-c2nc(-c3ccccc3)nc(-c3ccc4c(c3)C3c5ccccc5C4c4cc(-c5nc(-c6ccccc6)nc(-c6cccc7ccccc67)n5)ccc43)n2)cc1]

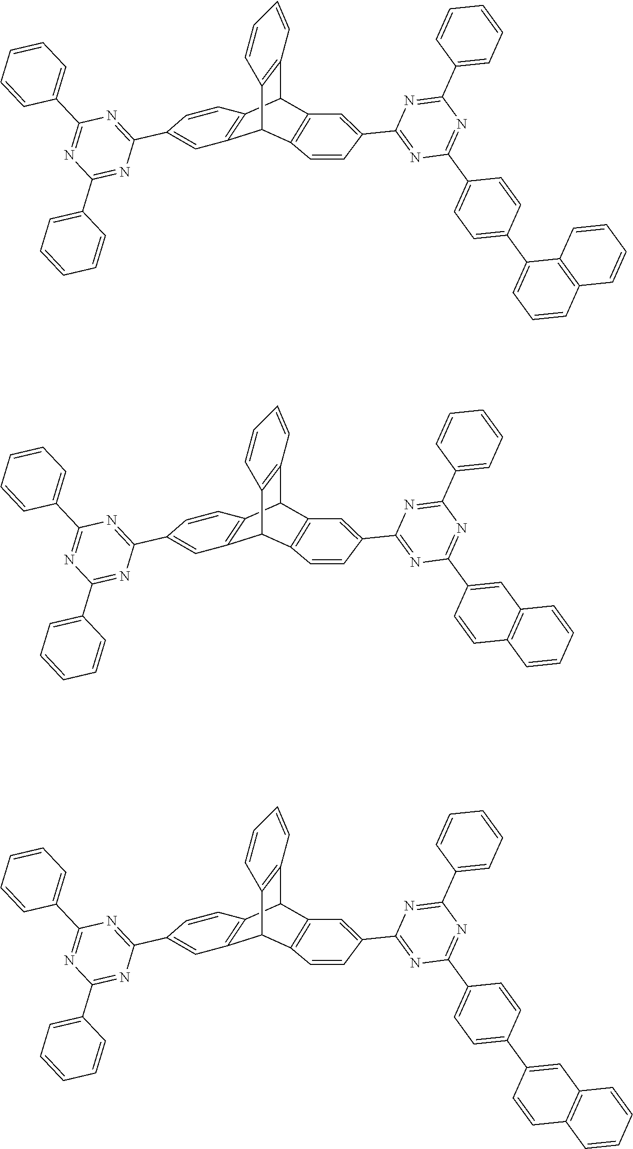 [SMILES: c1ccc(-c2nc(-c3ccccc3)nc(-c3ccc4c(c3)C3c5ccccc5C4c4cc(-c5nc(-c6ccccc6)nc(-c6ccc(-c7cccc8ccccc78)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc7ccccc7c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8ccccc8c7)cc6)n5)ccc43)n2)cc1]

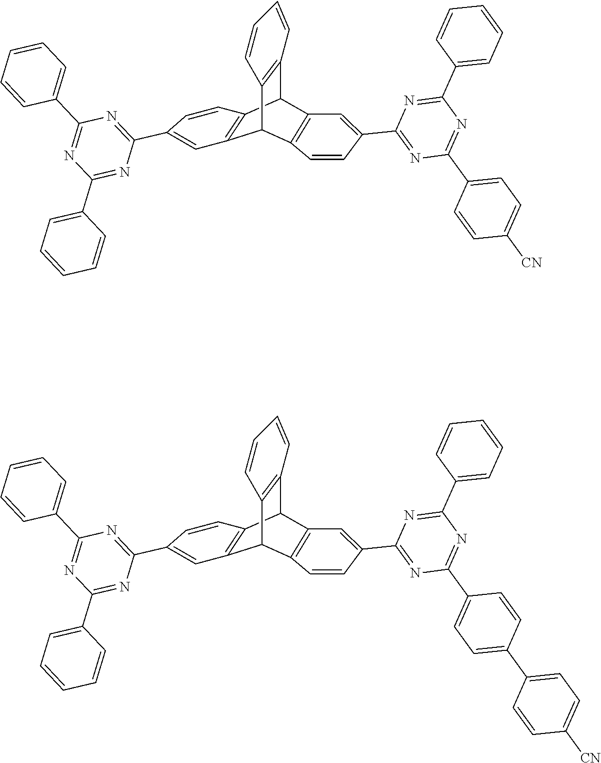 [SMILES: N#Cc1ccc(-c2nc(-c3ccccc3)nc(-c3ccc4c(c3)C3c5ccccc5C4c4cc(-c5nc(-c6ccccc6)nc(-c6ccccc6)n5)ccc43)n2)cc1 ; N#Cc1ccc(-c2ccc(-c3nc(-c4ccccc4)nc(-c4ccc5c(c4)C4c6ccccc6C5c5cc(-c6nc(-c7ccccc7)nc(-c7ccccc7)n6)ccc54)n3)cc2)cc1]

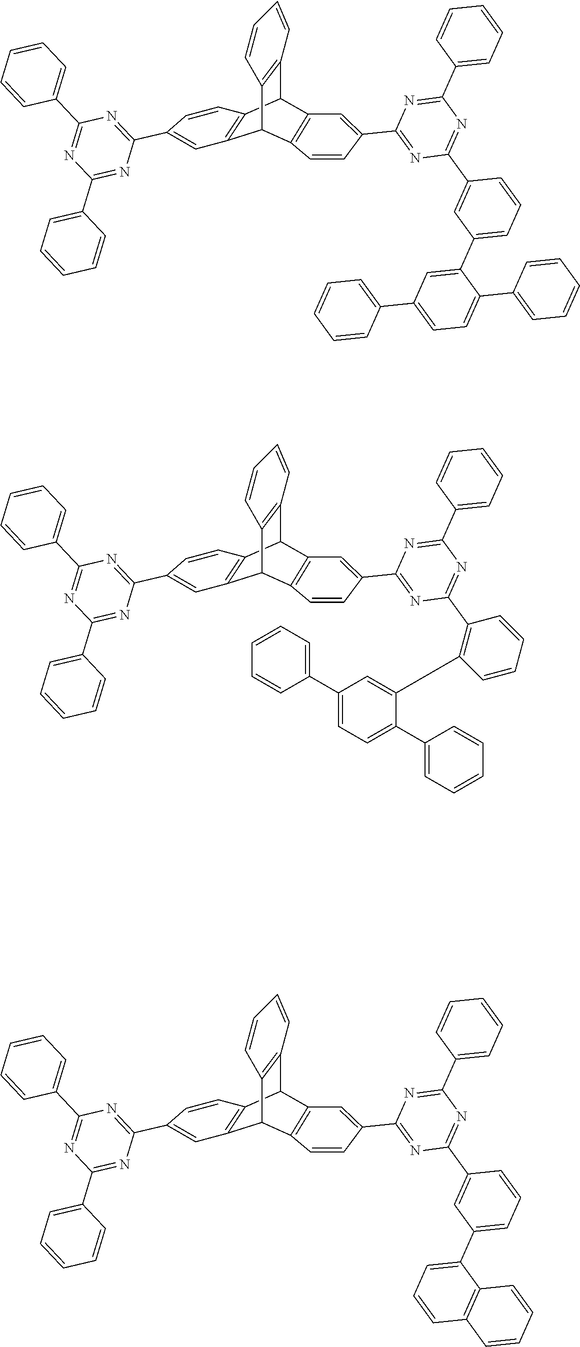 [SMILES: c1ccc(-c2ccc(-c3ccccc3)c(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)c2)cc1 ; c1ccc(-c2ccc(-c3ccccc3)c(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)c2)cc1 ; c1ccc(-c2nc(-c3ccccc3)nc(-c3ccc4c(c3)C3c5ccccc5C4c4cc(-c5nc(-c6ccccc6)nc(-c6cccc(-c7cccc8ccccc78)c6)n5)ccc43)n2)cc1]

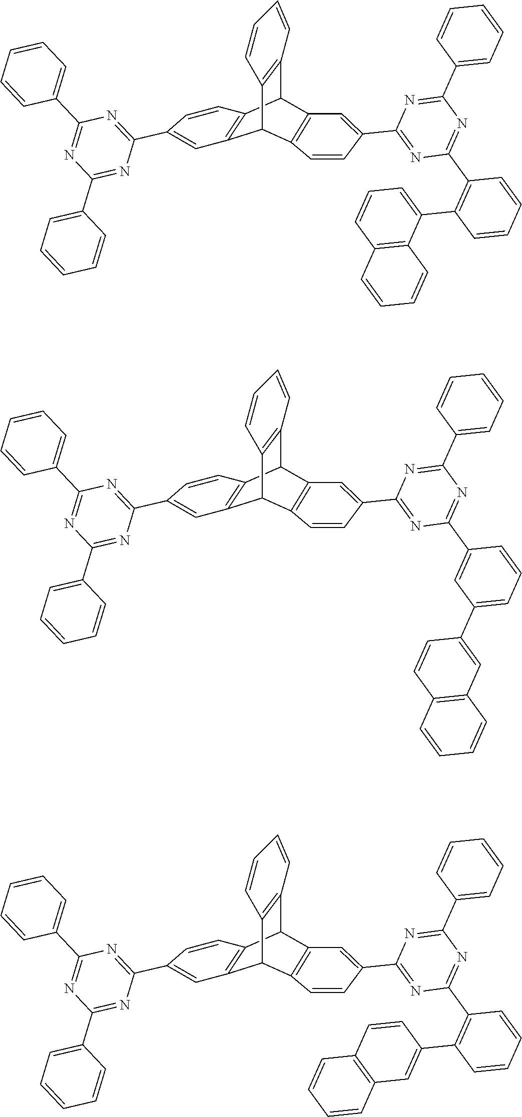 [SMILES: c1ccc(-c2nc(-c3ccccc3)nc(-c3ccc4c(c3)C3c5ccccc5C4c4cc(-c5nc(-c6ccccc6)nc(-c6ccccc6-c6cccc7ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6cccc(-c7ccc8ccccc8c7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7ccccc7c6)n5)ccc43)n2)cc1]

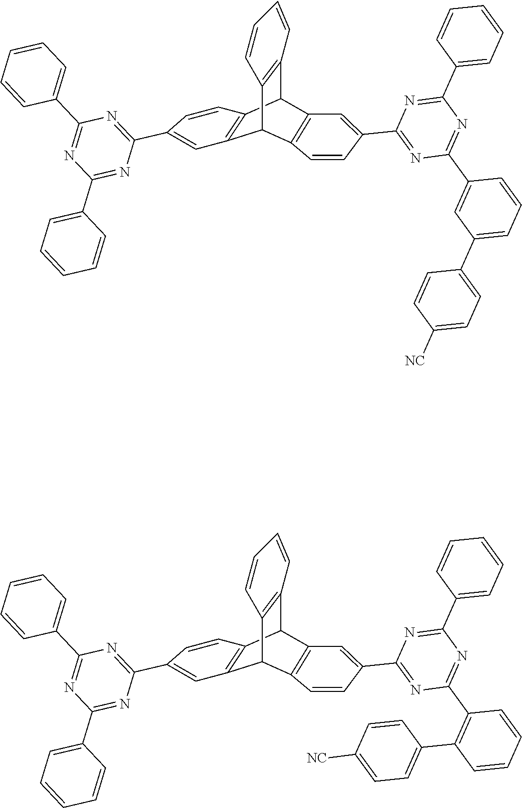 [SMILES: N#Cc1ccc(-c2cccc(-c3nc(-c4ccccc4)nc(-c4ccc5c(c4)C4c6ccccc6C5c5cc(-c6nc(-c7ccccc7)nc(-c7ccccc7)n6)ccc54)n3)c2)cc1 ; N#Cc1ccc(-c2ccccc2-c2nc(-c3ccccc3)nc(-c3ccc4c(c3)C3c5ccccc5C4c4cc(-c5nc(-c6ccccc6)nc(-c6ccccc6)n5)ccc43)n2)cc1]

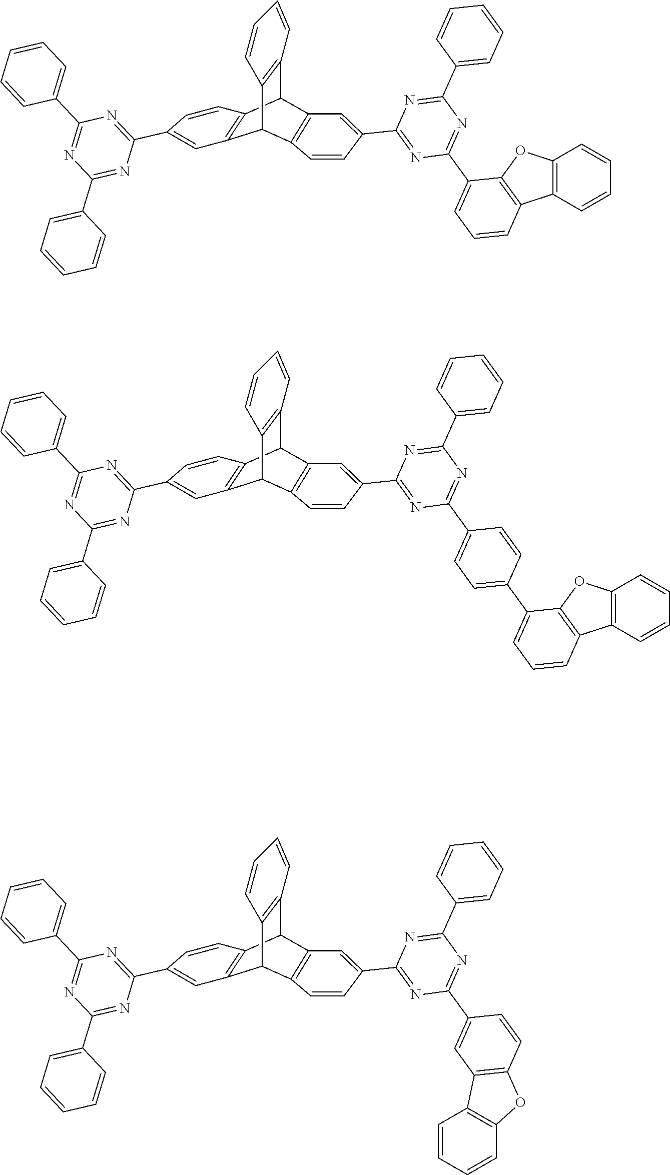 [SMILES: c1ccc(-c2nc(-c3ccccc3)nc(-c3ccc4c(c3)C3c5ccccc5C4c4cc(-c5nc(-c6ccccc6)nc(-c6cccc7c6oc6ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c7oc7ccccc78)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc7oc8ccccc8c7c6)n5)ccc43)n2)cc1]

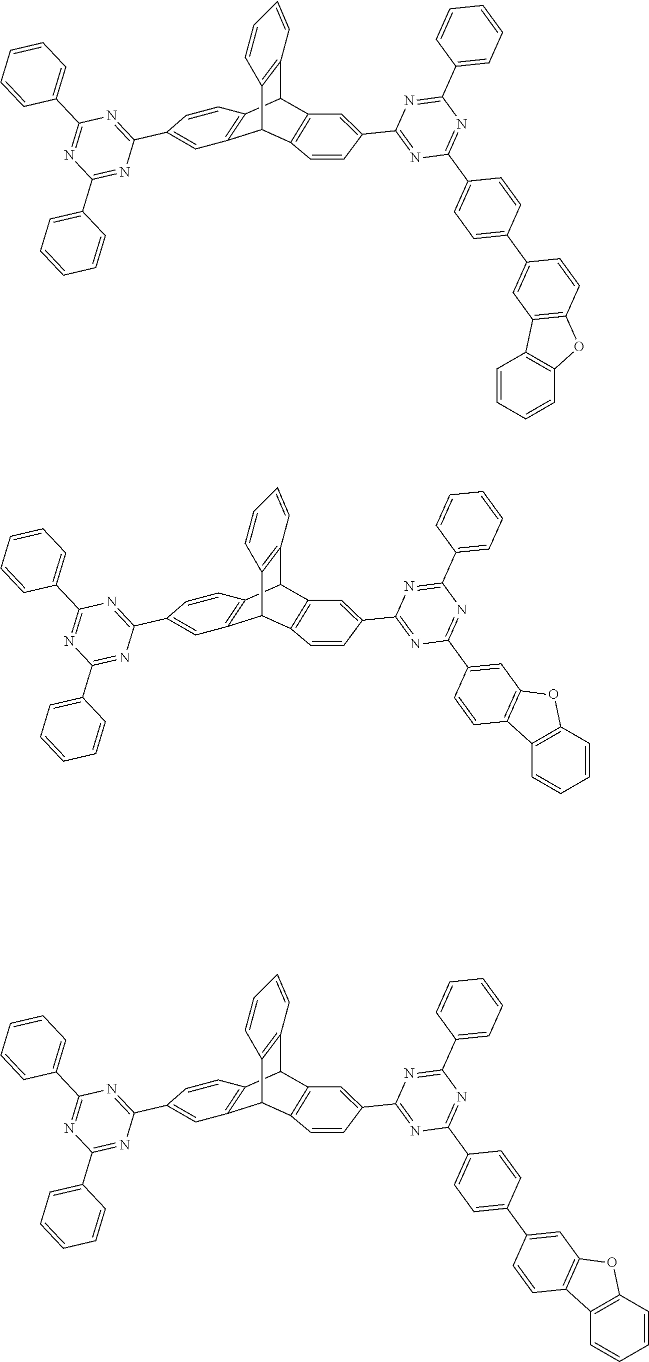 [SMILES: c1ccc(-c2nc(-c3ccccc3)nc(-c3ccc4c(c3)C3c5ccccc5C4c4cc(-c5nc(-c6ccccc6)nc(-c6ccc(-c7ccc8oc9ccccc9c8c7)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc7c(c6)oc6ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c(c7)oc7ccccc78)cc6)n5)ccc43)n2)cc1]

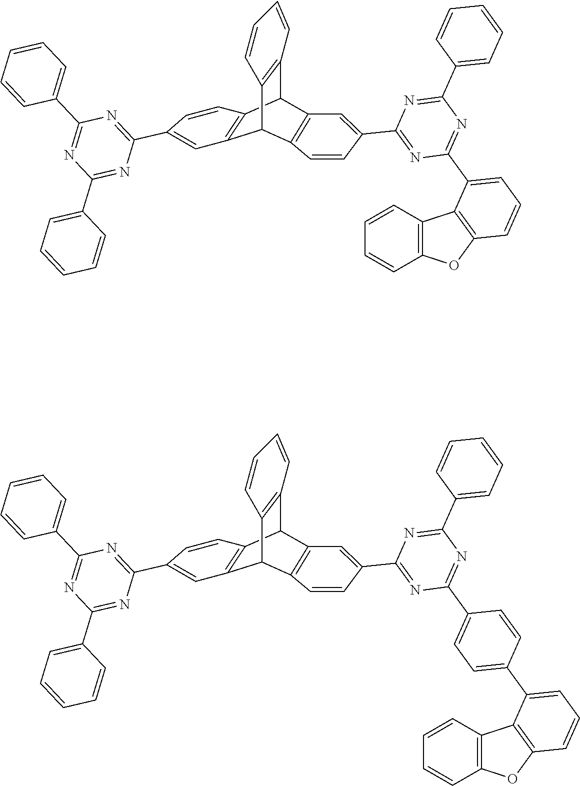 [SMILES: c1ccc(-c2nc(-c3ccccc3)nc(-c3ccc4c(c3)C3c5ccccc5C4c4cc(-c5nc(-c6ccccc6)nc(-c6cccc7oc8ccccc8c67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8oc9ccccc9c78)cc6)n5)ccc43)n2)cc1]

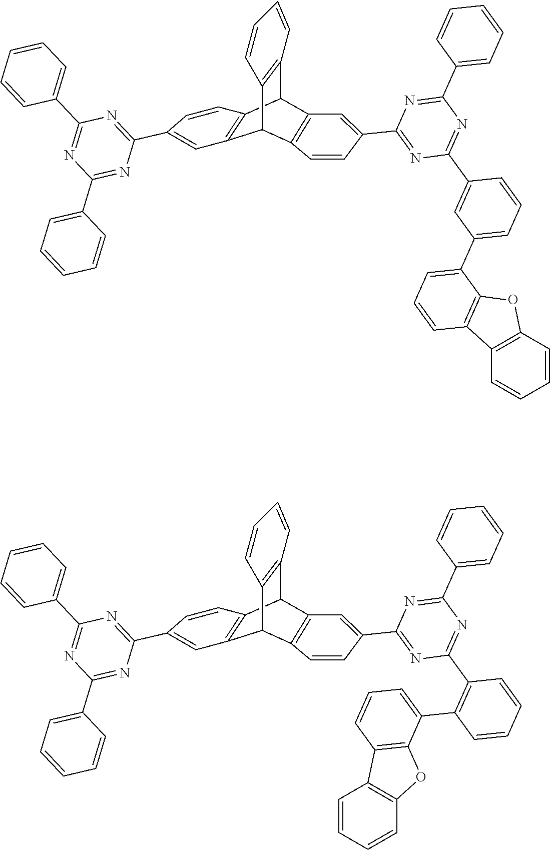 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c7oc7ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c6oc6ccccc67)n5)ccc43)n2)cc1]

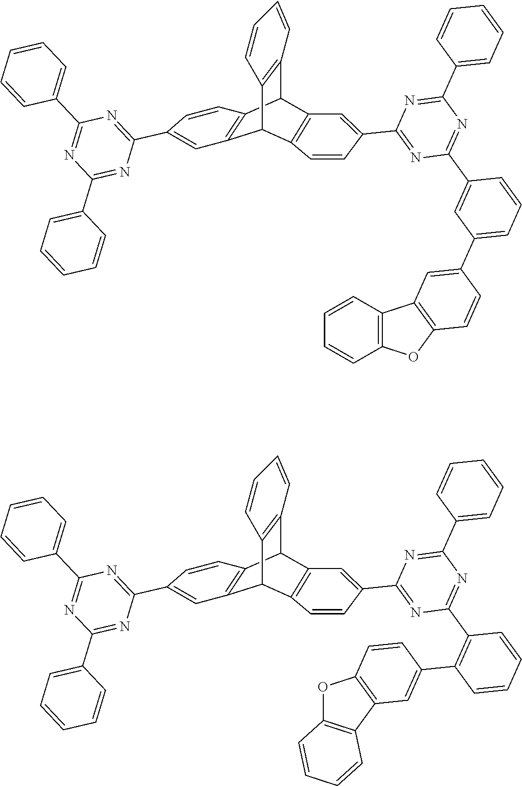 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8oc9ccccc9c8c7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7oc8ccccc8c7c6)n5)ccc43)n2)cc1]

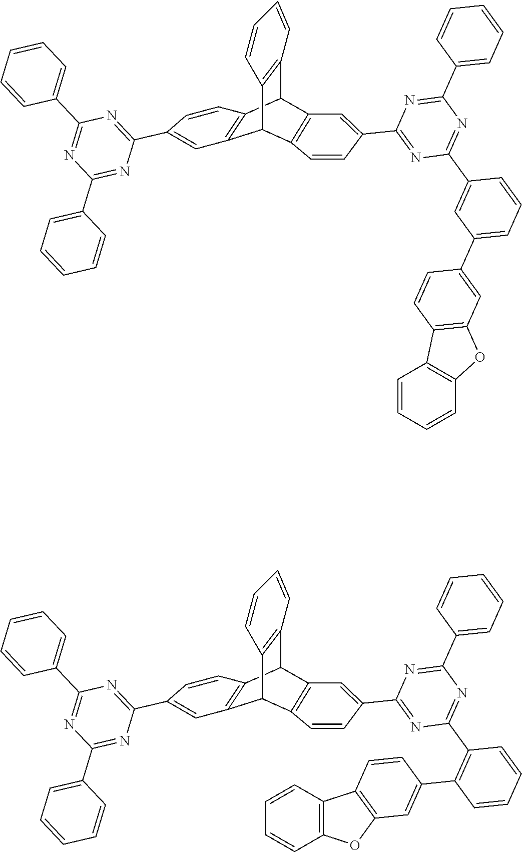 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c(c7)oc7ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c(c6)oc6ccccc67)n5)ccc43)n2)cc1]

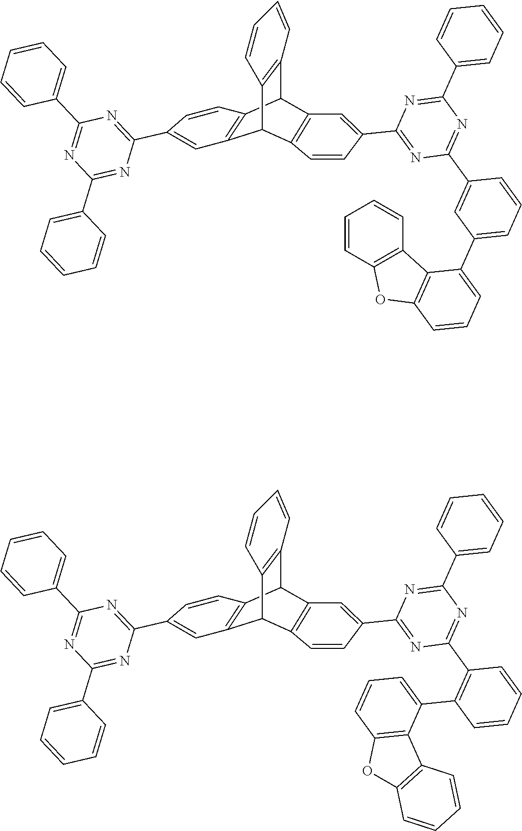 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8oc9ccccc9c78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7oc8ccccc8c67)n5)ccc43)n2)cc1]

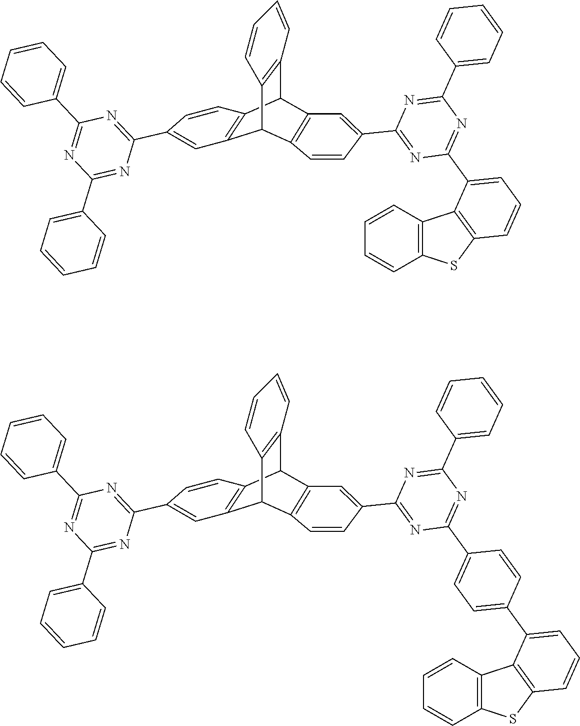 [SMILES: c1ccc(-c2nc(-c3ccccc3)nc(-c3ccc4c(c3)C3c5ccccc5C4c4cc(-c5nc(-c6ccccc6)nc(-c6cccc7sc8ccccc8c67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8sc9ccccc9c78)cc6)n5)ccc43)n2)cc1]

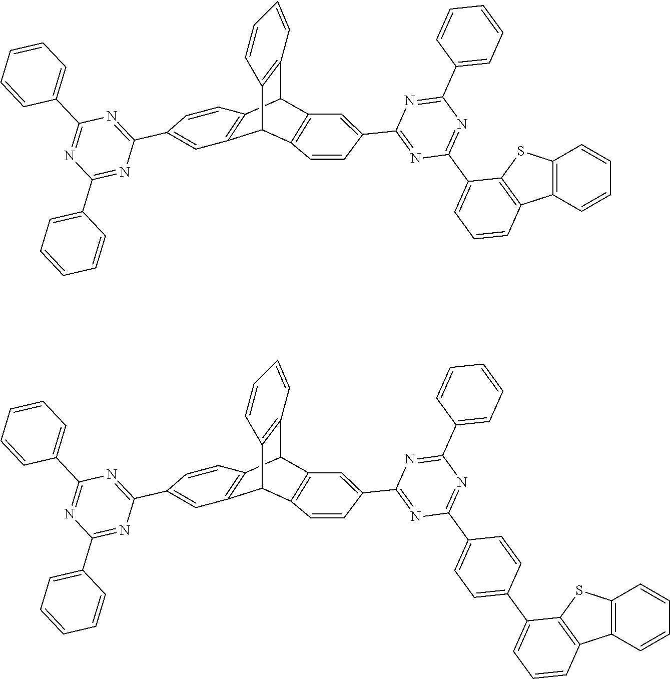 [SMILES: c1ccc(-c2nc(-c3ccccc3)nc(-c3ccc4c(c3)C3c5ccccc5C4c4cc(-c5nc(-c6ccccc6)nc(-c6cccc7c6sc6ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c7sc7ccccc78)cc6)n5)ccc43)n2)cc1]

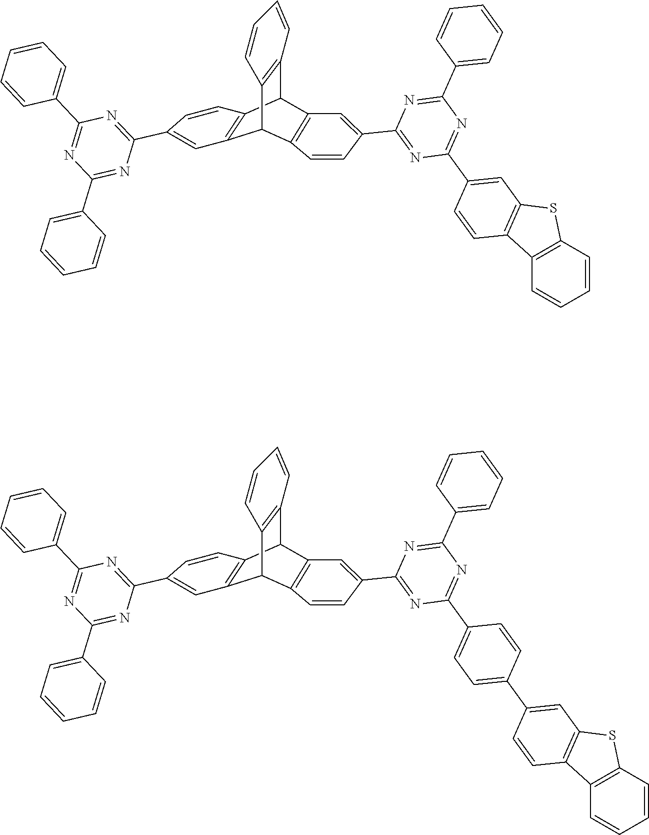 [SMILES: c1ccc(-c2nc(-c3ccccc3)nc(-c3ccc4c(c3)C3c5ccccc5C4c4cc(-c5nc(-c6ccccc6)nc(-c6ccc7c(c6)sc6ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c(c7)sc7ccccc78)cc6)n5)ccc43)n2)cc1]

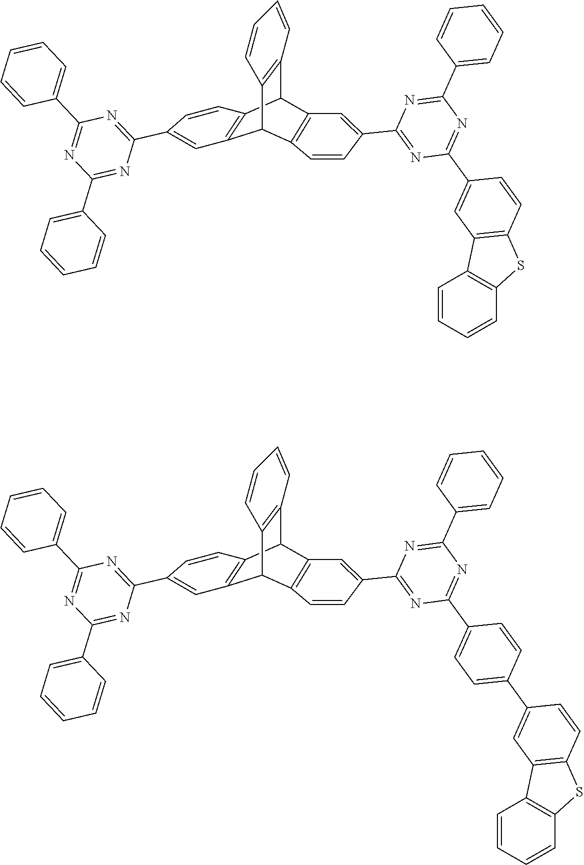 [SMILES: c1ccc(-c2nc(-c3ccccc3)nc(-c3ccc4c(c3)C3c5ccccc5C4c4cc(-c5nc(-c6ccccc6)nc(-c6ccc7sc8ccccc8c7c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8sc9ccccc9c8c7)cc6)n5)ccc43)n2)cc1]

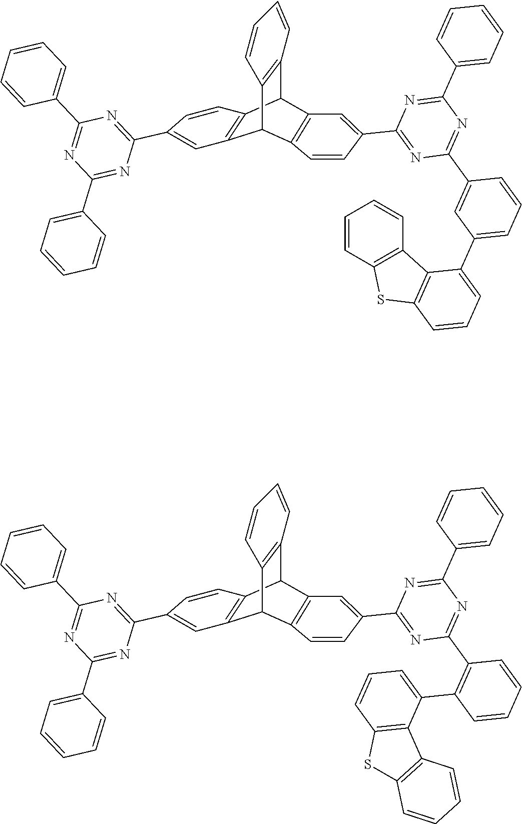 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8sc9ccccc9c78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7sc8ccccc8c67)n5)ccc43)n2)cc1]

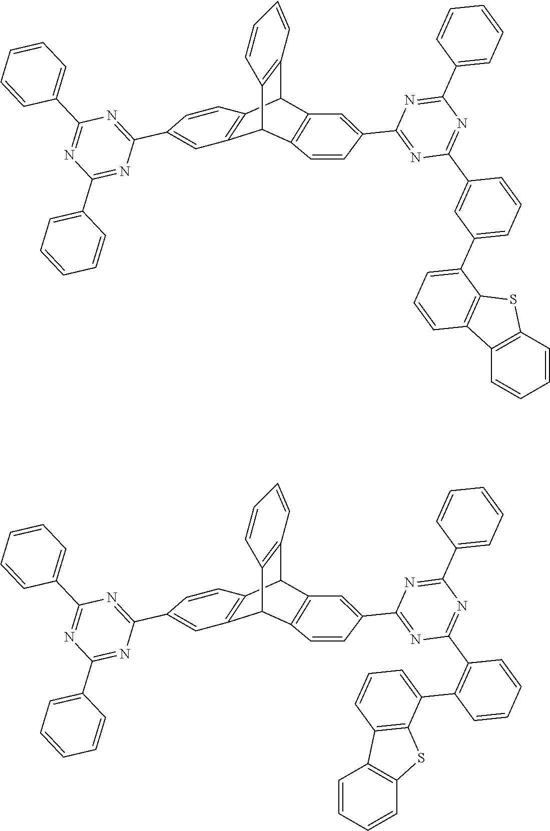 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c7sc7ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c6sc6ccccc67)n5)ccc43)n2)cc1]

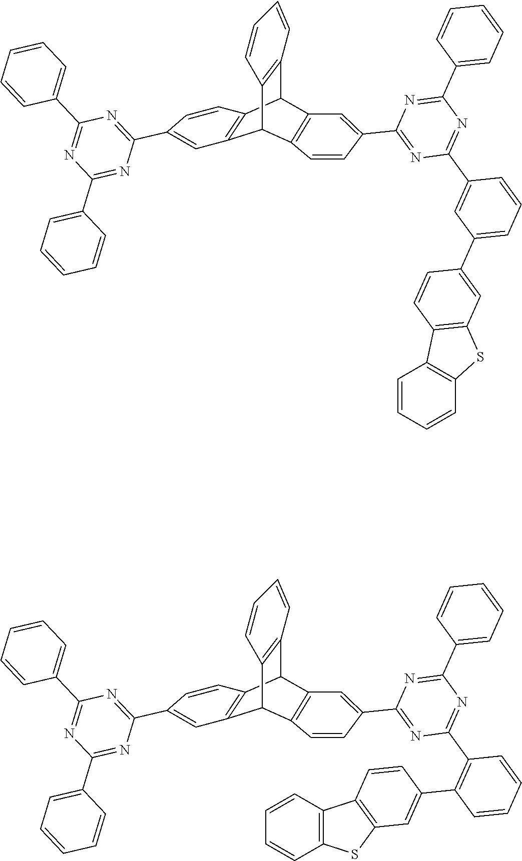 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c(c7)sc7ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c(c6)sc6ccccc67)n5)ccc43)n2)cc1]

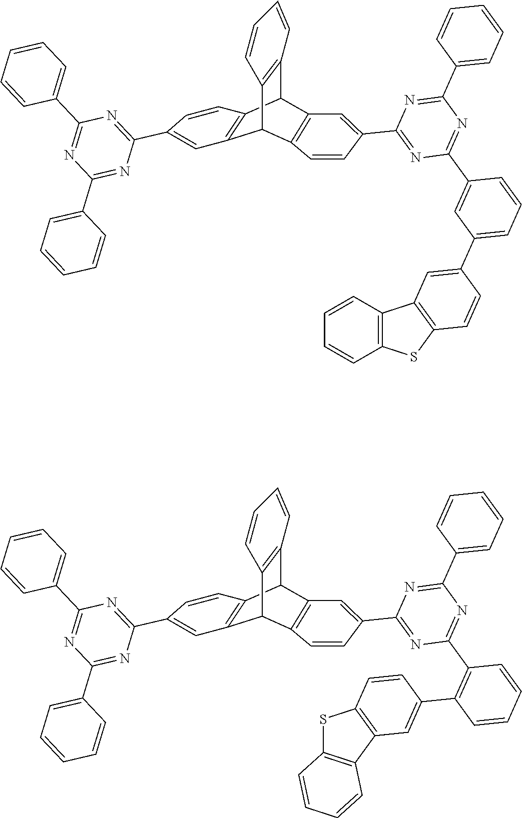 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8sc9ccccc9c8c7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7sc8ccccc8c7c6)n5)ccc43)n2)cc1]

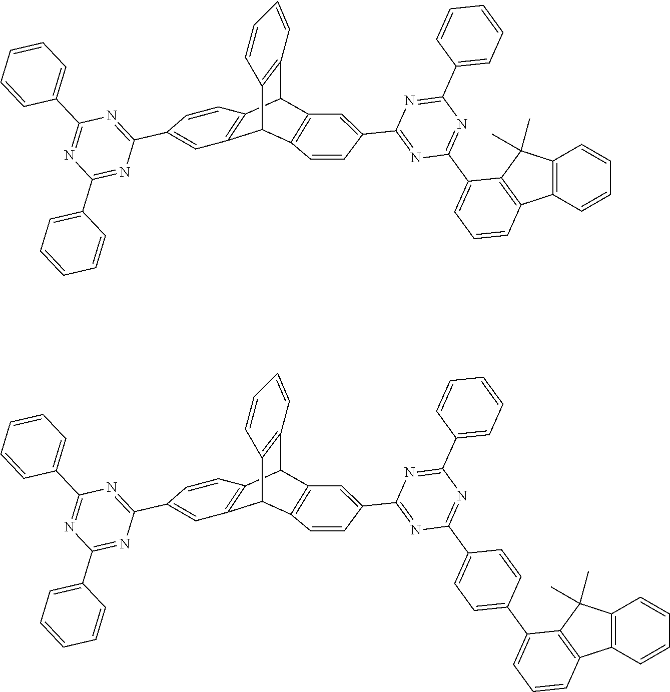 [SMILES: CC1(C)c2ccccc2-c2cccc(-c3nc(-c4ccccc4)nc(-c4ccc5c(c4)C4c6ccccc6C5c5cc(-c6nc(-c7ccccc7)nc(-c7ccccc7)n6)ccc54)n3)c21 ; CC1(C)c2ccccc2-c2cccc(-c3ccc(-c4nc(-c5ccccc5)nc(-c5ccc6c(c5)C5c7ccccc7C6c6cc(-c7nc(-c8ccccc8)nc(-c8ccccc8)n7)ccc65)n4)cc3)c21]

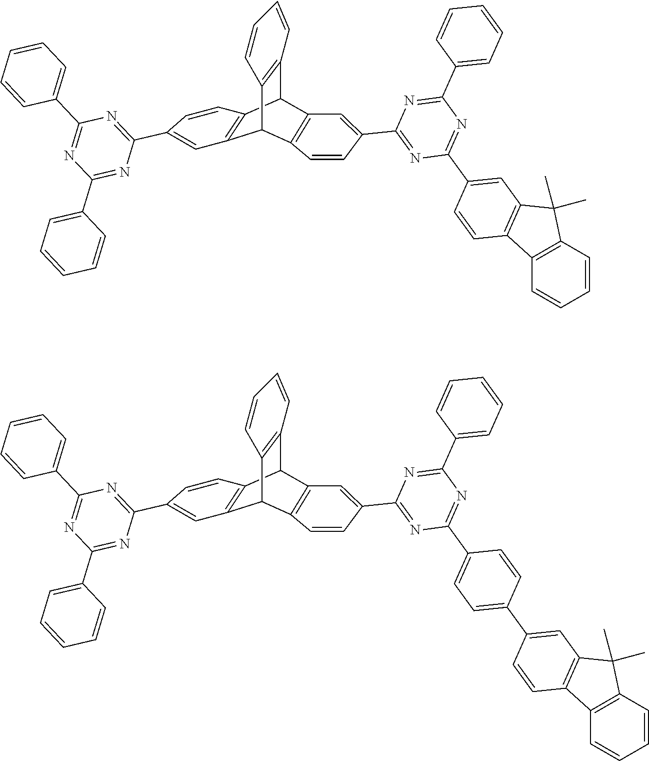 [SMILES: CC1(C)c2ccccc2-c2ccc(-c3nc(-c4ccccc4)nc(-c4ccc5c(c4)C4c6ccccc6C5c5cc(-c6nc(-c7ccccc7)nc(-c7ccccc7)n6)ccc54)n3)cc21 ; CC1(C)c2ccccc2-c2ccc(-c3ccc(-c4nc(-c5ccccc5)nc(-c5ccc6c(c5)C5c7ccccc7C6c6cc(-c7nc(-c8ccccc8)nc(-c8ccccc8)n7)ccc65)n4)cc3)cc21]

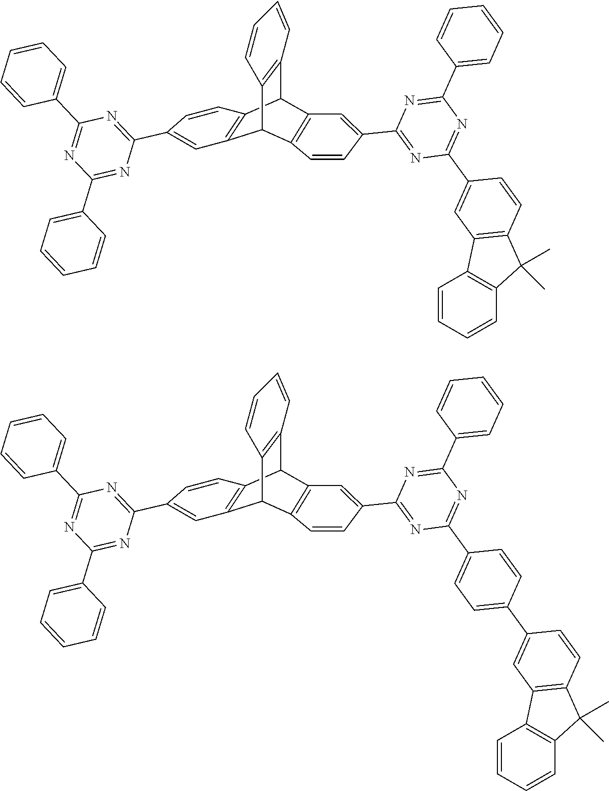

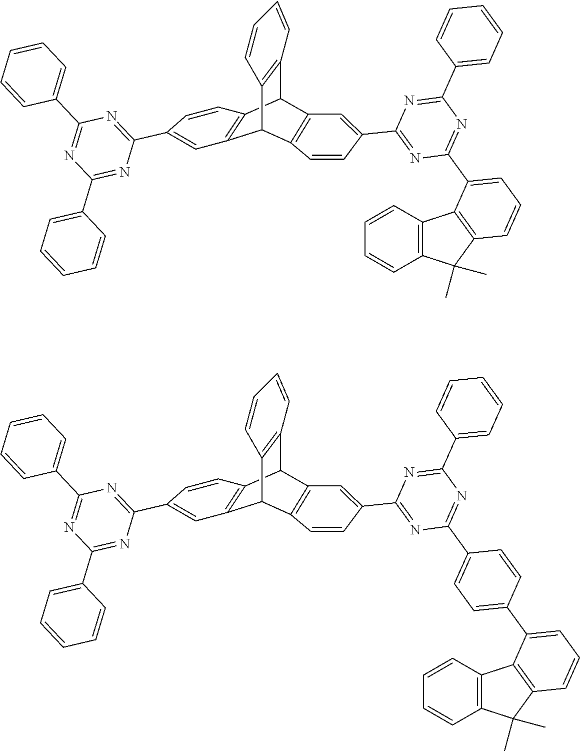 [SMILES: CC1(C)c2ccccc2-c2c(-c3nc(-c4ccccc4)nc(-c4ccc5c(c4)C4c6ccccc6C5c5cc(-c6nc(-c7ccccc7)nc(-c7ccccc7)n6)ccc54)n3)cccc21 ; CC1(C)c2ccccc2-c2c(-c3ccc(-c4nc(-c5ccccc5)nc(-c5ccc6c(c5)C5c7ccccc7C6c6cc(-c7nc(-c8ccccc8)nc(-c8ccccc8)n7)ccc65)n4)cc3)cccc21]

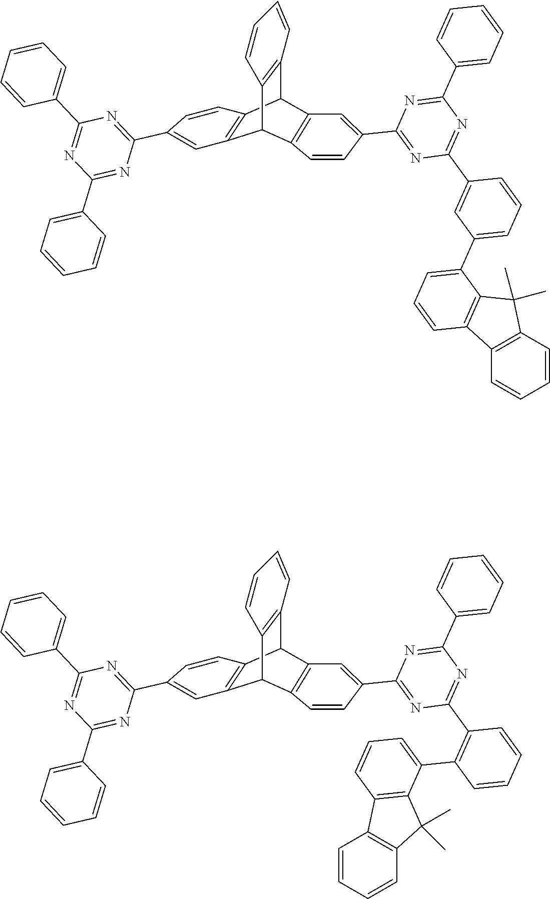 [SMILES: CC1(C)c2ccccc2-c2cccc(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)c21 ; CC1(C)c2ccccc2-c2cccc(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)c21]

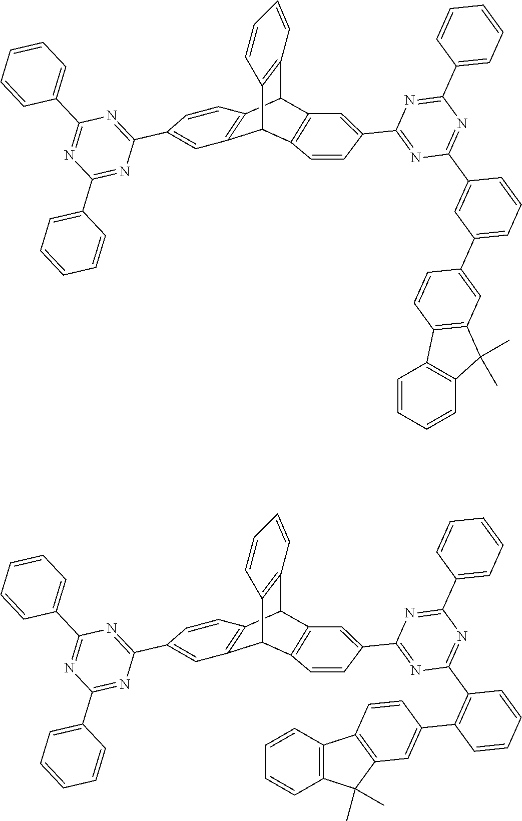 [SMILES: CC1(C)c2ccccc2-c2ccc(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)cc21 ; CC1(C)c2ccccc2-c2ccc(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)cc21]

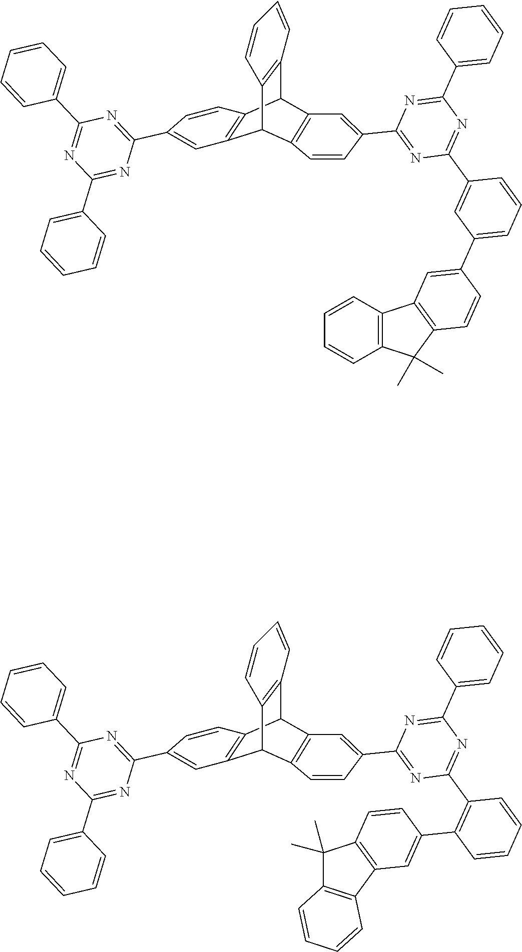 [SMILES: CC1(C)c2ccccc2-c2cc(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)ccc21 ; CC1(C)c2ccccc2-c2cc(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)ccc21]

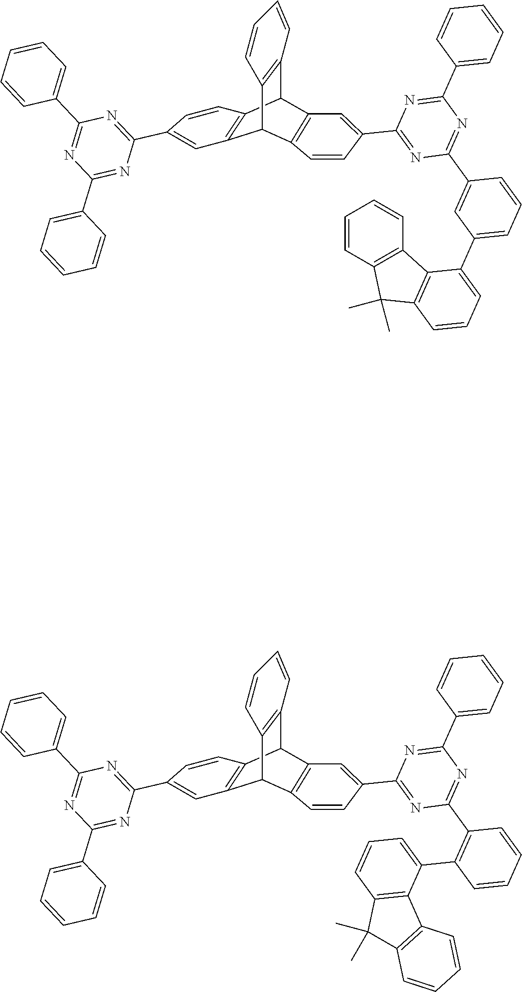 [SMILES: CC1(C)c2ccccc2-c2c(-c3cccc(-c4nc(-c5ccccc5)nc(-c5ccc6c(c5)C5c7ccccc7C6c6cc(-c7nc(-c8ccccc8)nc(-c8ccccc8)n7)ccc65)n4)c3)cccc21 ; CC1(C)c2ccccc2-c2c(-c3ccccc3-c3nc(-c4ccccc4)nc(-c4ccc5c(c4)C4c6ccccc6C5c5cc(-c6nc(-c7ccccc7)nc(-c7ccccc7)n6)ccc54)n3)cccc21]

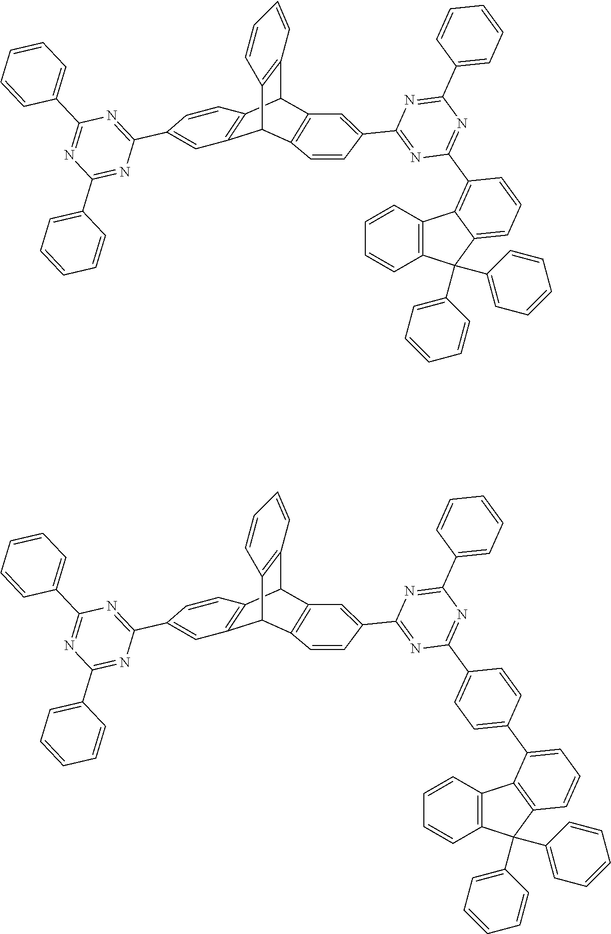 [SMILES: c1ccc(-c2nc(-c3ccccc3)nc(-c3ccc4c(c3)C3c5ccccc5C4c4cc(-c5nc(-c6ccccc6)nc(-c6cccc7c6-c6ccccc6C7(c6ccccc6)c6ccccc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c7-c7ccccc7C8(c7ccccc7)c7ccccc7)cc6)n5)ccc43)n2)cc1]

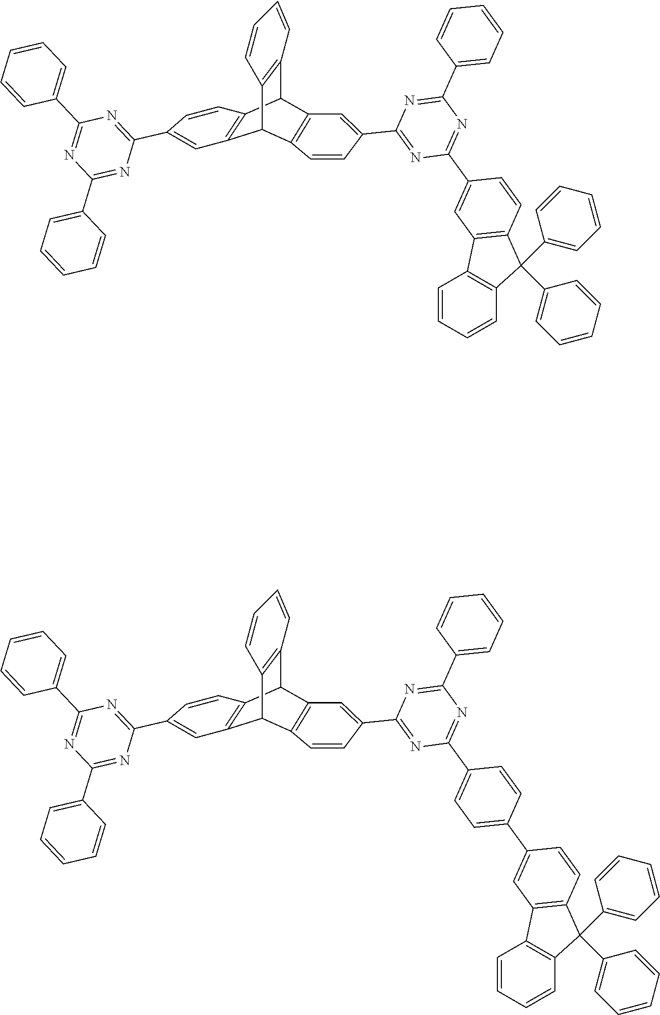 [SMILES: c1ccc(-c2nc(-c3ccccc3)nc(-c3ccc4c(c3)C3c5ccccc5C4c4cc(-c5nc(-c6ccccc6)nc(-c6ccc7c(c6)-c6ccccc6C7(c6ccccc6)c6ccccc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c(c7)-c7ccccc7C8(c7ccccc7)c7ccccc7)cc6)n5)ccc43)n2)cc1]

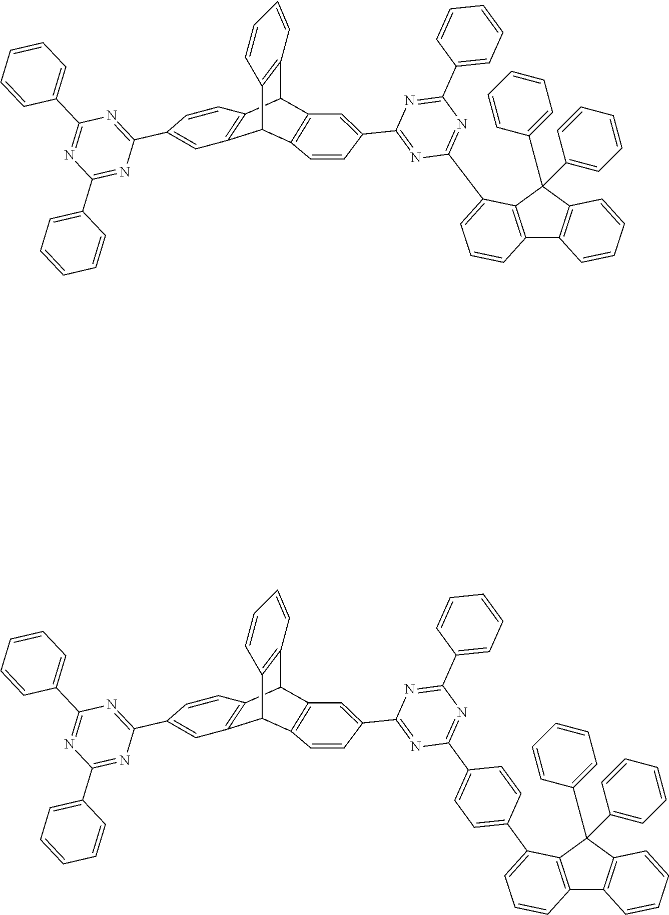 [SMILES: c1ccc(-c2nc(-c3ccccc3)nc(-c3ccc4c(c3)C3c5ccccc5C4c4cc(-c5nc(-c6ccccc6)nc(-c6cccc7c6C(c6ccccc6)(c6ccccc6)c6ccccc6-7)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c7C(c7ccccc7)(c7ccccc7)c7ccccc7-8)cc6)n5)ccc43)n2)cc1]

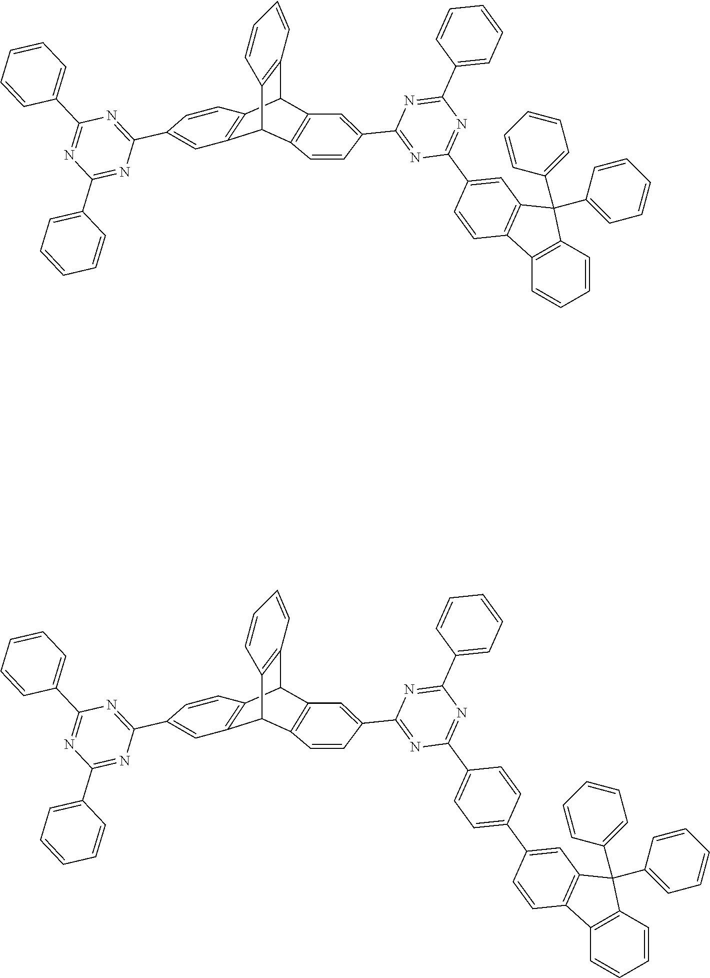 [SMILES: c1ccc(-c2nc(-c3ccccc3)nc(-c3ccc4c(c3)C3c5ccccc5C4c4cc(-c5nc(-c6ccccc6)nc(-c6ccc7c(c6)C(c6ccccc6)(c6ccccc6)c6ccccc6-7)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c(c7)C(c7ccccc7)(c7ccccc7)c7ccccc7-8)cc6)n5)ccc43)n2)cc1]

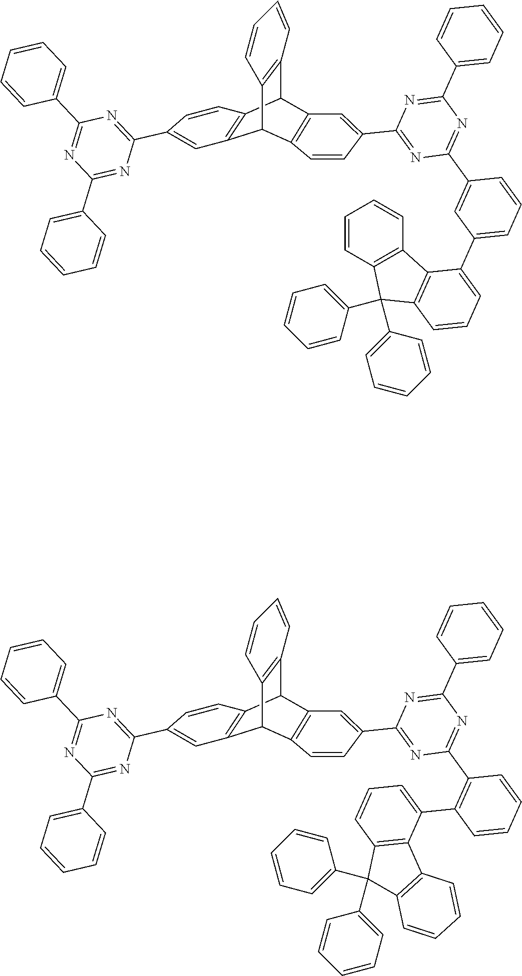 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c7-c7ccccc7C8(c7ccccc7)c7ccccc7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c6-c6ccccc6C7(c6ccccc6)c6ccccc6)n5)ccc43)n2)cc1]

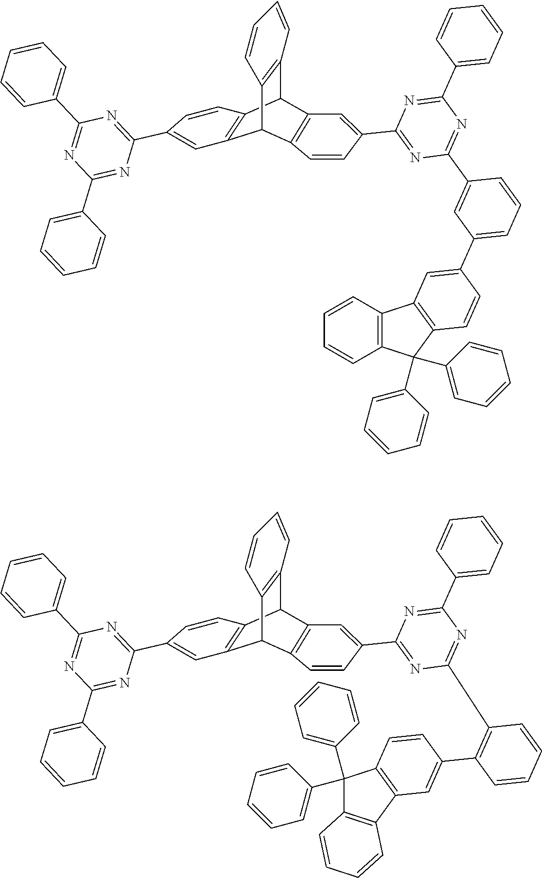 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c(c7)-c7ccccc7C8(c7ccccc7)c7ccccc7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c(c6)-c6ccccc6C7(c6ccccc6)c6ccccc6)n5)ccc43)n2)cc1]

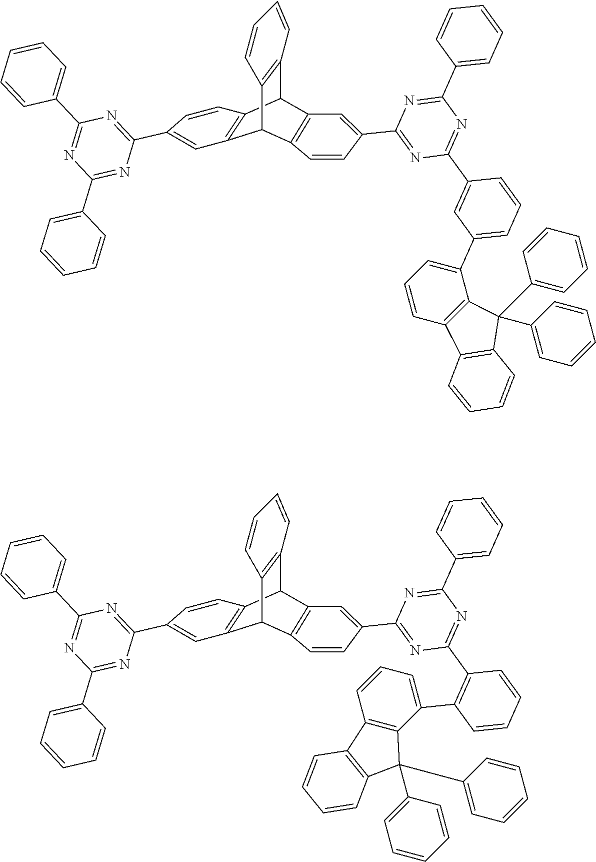 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c7C(c7ccccc7)(c7ccccc7)c7ccccc7-8)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c6C(c6ccccc6)(c6ccccc6)c6ccccc6-7)n5)ccc43)n2)cc1]

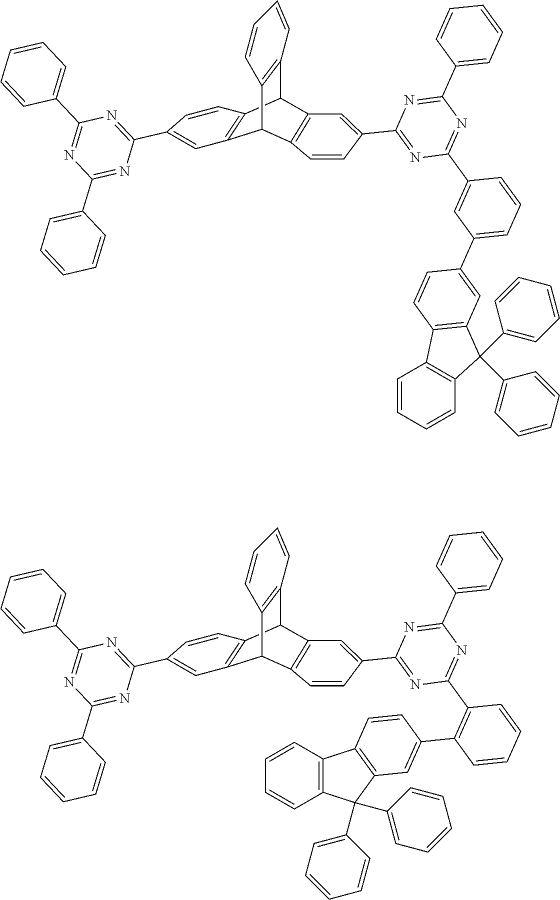 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c(c7)C(c7ccccc7)(c7ccccc7)c7ccccc7-8)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c(c6)C(c6ccccc6)(c6ccccc6)c6ccccc6-7)n5)ccc43)n2)cc1]

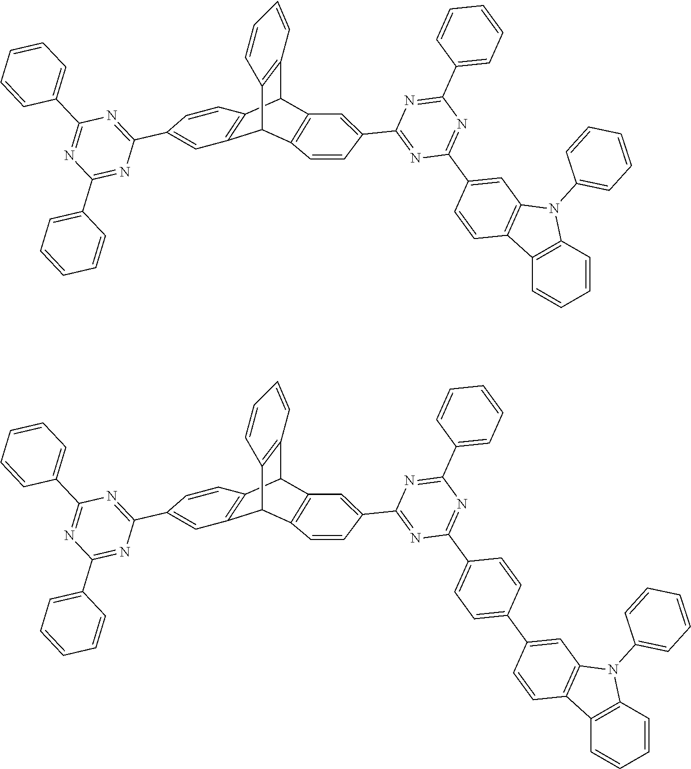 [SMILES: c1ccc(-c2nc(-c3ccccc3)nc(-c3ccc4c(c3)C3c5ccccc5C4c4cc(-c5nc(-c6ccccc6)nc(-c6ccc7c8ccccc8n(-c8ccccc8)c7c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c9ccccc9n(-c9ccccc9)c8c7)cc6)n5)ccc43)n2)cc1]

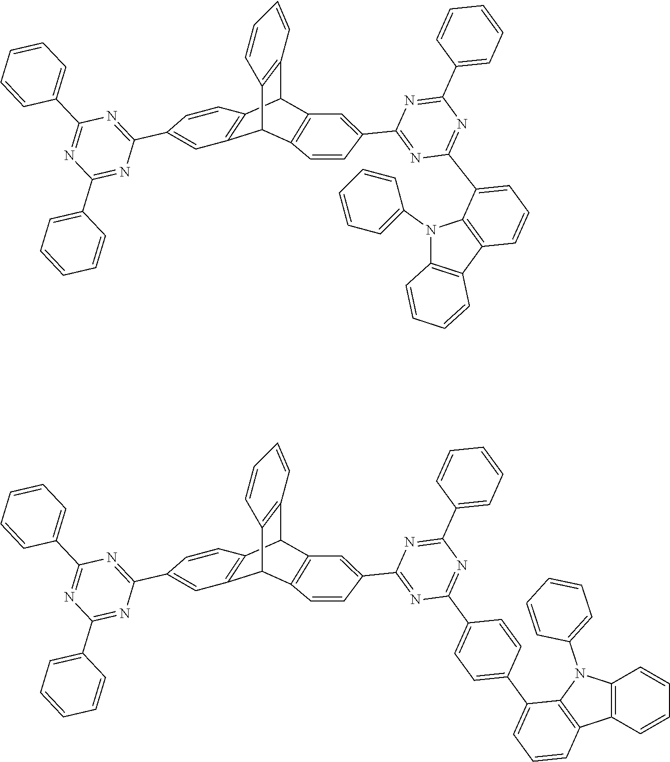 [SMILES: c1ccc(-c2nc(-c3ccccc3)nc(-c3ccc4c(c3)C3c5ccccc5C4c4cc(-c5nc(-c6ccccc6)nc(-c6cccc7c8ccccc8n(-c8ccccc8)c67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c9ccccc9n(-c9ccccc9)c78)cc6)n5)ccc43)n2)cc1]

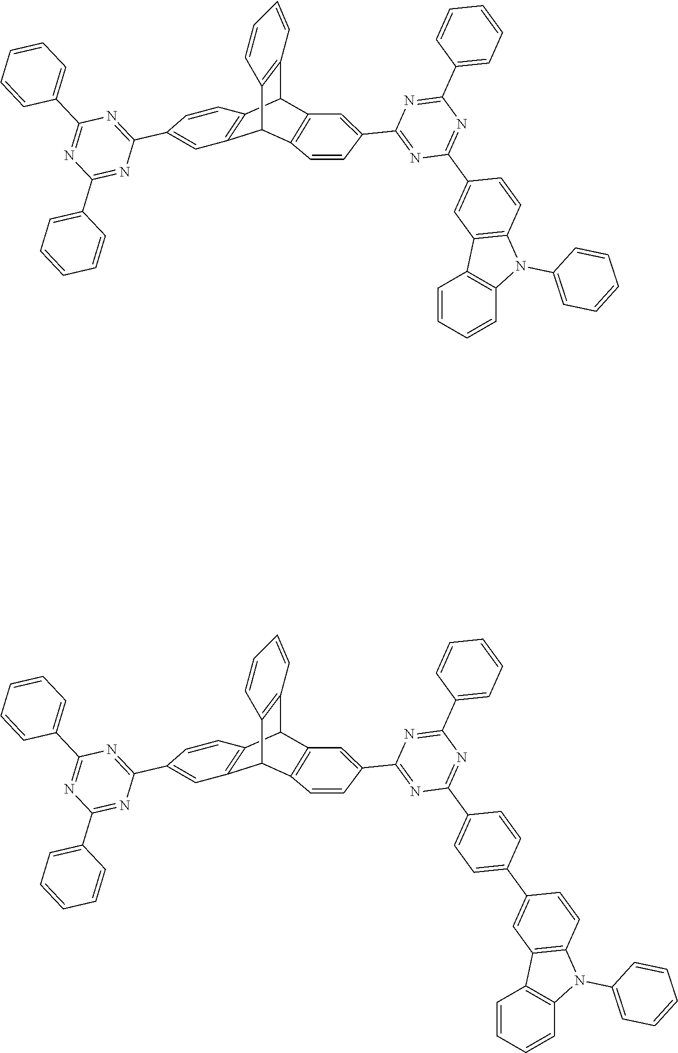 [SMILES: c1ccc(-c2nc(-c3ccccc3)nc(-c3ccc4c(c3)C3c5ccccc5C4c4cc(-c5nc(-c6ccccc6)nc(-c6ccc7c(c6)c6ccccc6n7-c6ccccc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8c(c7)c7ccccc7n8-c7ccccc7)cc6)n5)ccc43)n2)cc1]

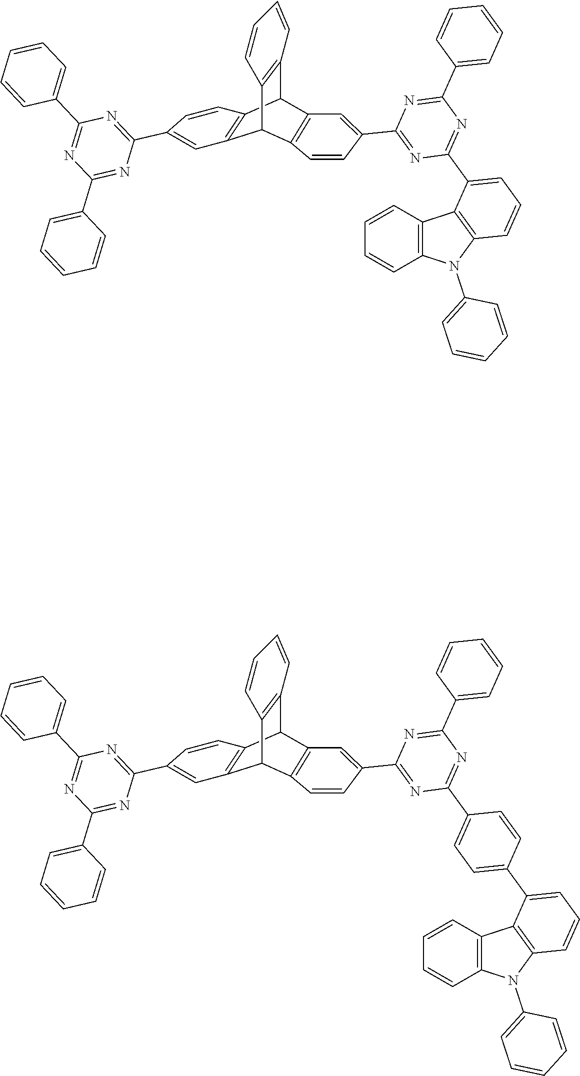 [SMILES: c1ccc(-c2nc(-c3ccccc3)nc(-c3ccc4c(c3)C3c5ccccc5C4c4cc(-c5nc(-c6ccccc6)nc(-c6cccc7c6c6ccccc6n7-c6ccccc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccc8c7c7ccccc7n8-c7ccccc7)cc6)n5)ccc43)n2)cc1]

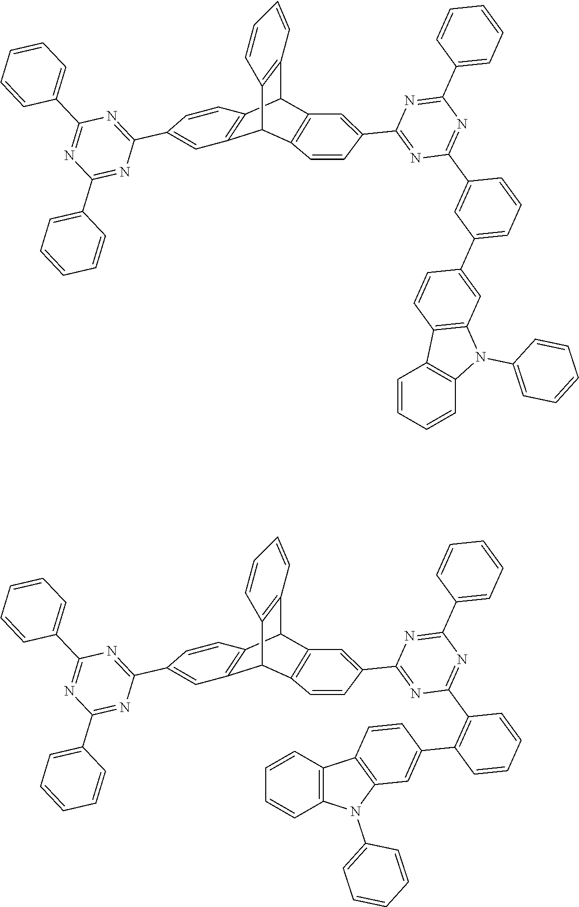 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c9ccccc9n(-c9ccccc9)c8c7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c8ccccc8n(-c8ccccc8)c7c6)n5)ccc43)n2)cc1]

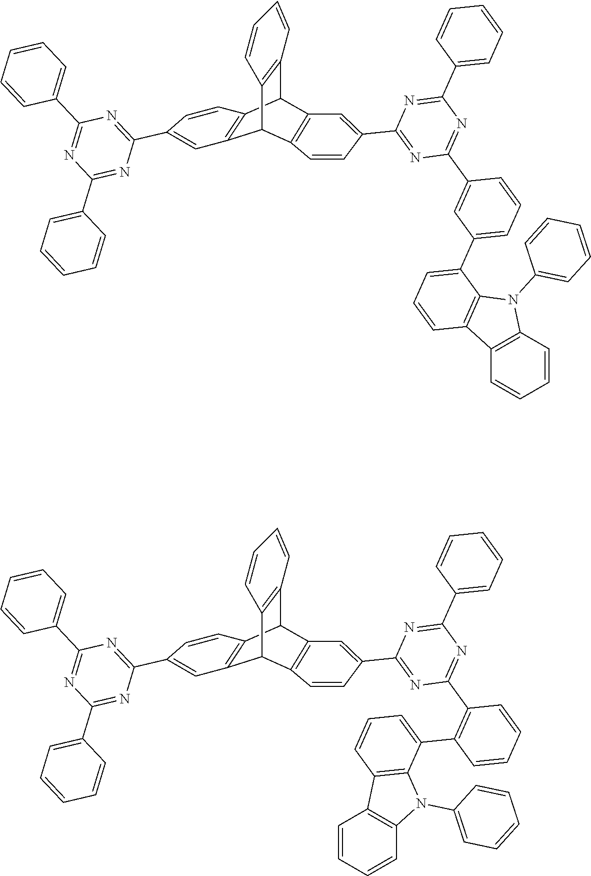 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c9ccccc9n(-c9ccccc9)c78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c8ccccc8n(-c8ccccc8)c67)n5)ccc43)n2)cc1]

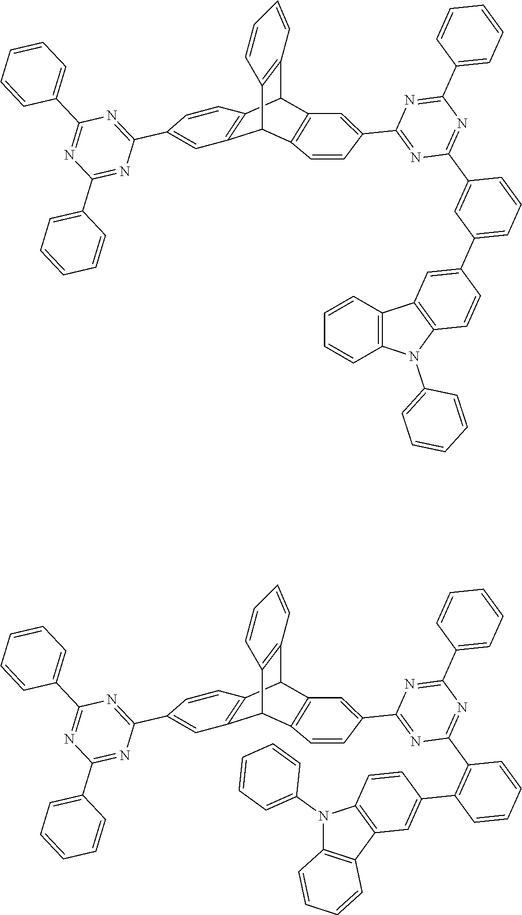 [SMILES: c1ccc(-c2nc(-c3ccccc3)nc(-c3ccc4c(c3)C3c5ccccc5C4c4cc(-c5nc(-c6ccccc6)nc(-c6cccc(-c7ccc8c(c7)c7ccccc7n8-c7ccccc7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7c(c6)c6ccccc6n7-c6ccccc6)n5)ccc43)n2)cc1]

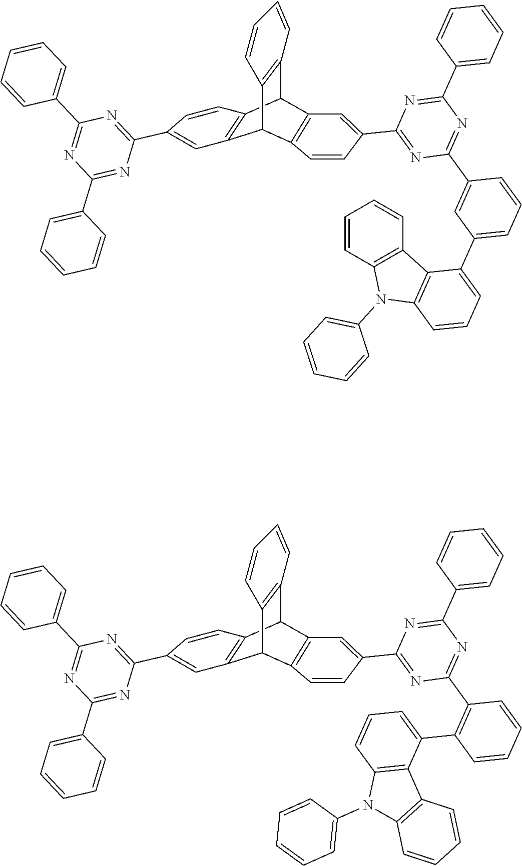 [SMILES: c1ccc(-c2nc(-c3ccccc3)nc(-c3ccc4c(c3)C3c5ccccc5C4c4cc(-c5nc(-c6ccccc6)nc(-c6cccc(-c7cccc8c7c7ccccc7n8-c7ccccc7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccc7c6c6ccccc6n7-c6ccccc6)n5)ccc43)n2)cc1]

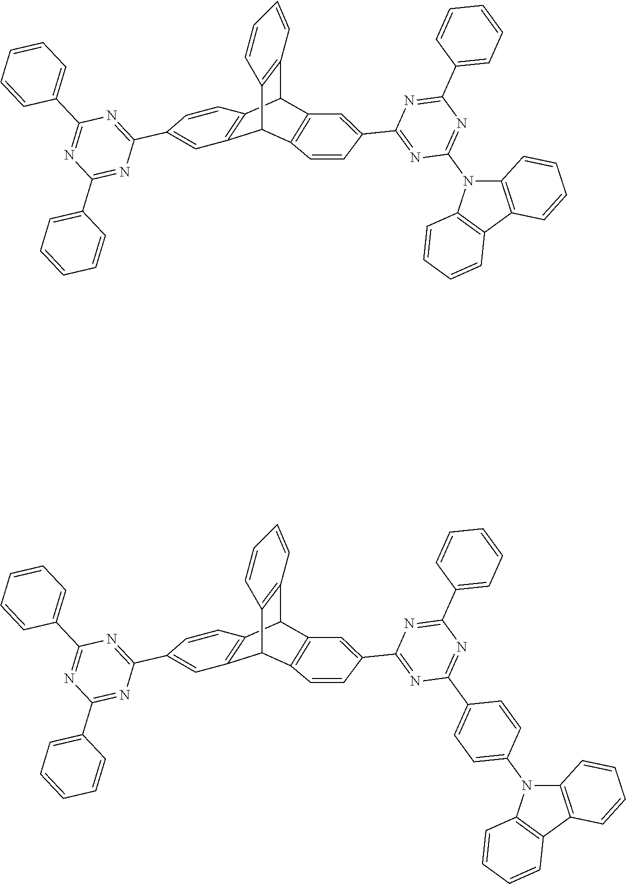 [SMILES: c1ccc(-c2nc(-c3ccccc3)nc(-c3ccc4c(c3)C3c5ccccc5C4c4cc(-c5nc(-c6ccccc6)nc(-n6c7ccccc7c7ccccc76)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-n7c8ccccc8c8ccccc87)cc6)n5)ccc43)n2)cc1]

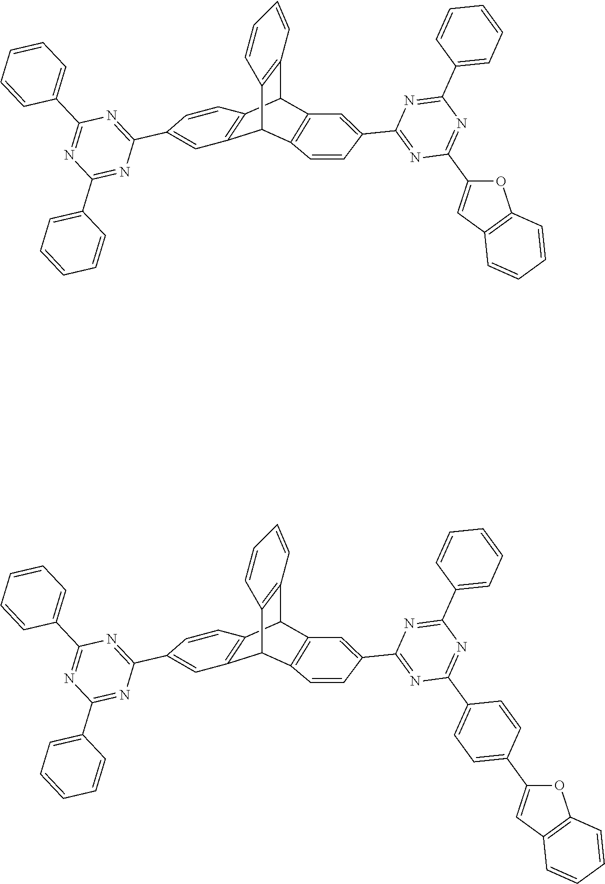 [SMILES: c1ccc(-c2nc(-c3ccccc3)nc(-c3ccc4c(c3)C3c5ccccc5C4c4cc(-c5nc(-c6ccccc6)nc(-c6cc7ccccc7o6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cc8ccccc8o7)cc6)n5)ccc43)n2)cc1]

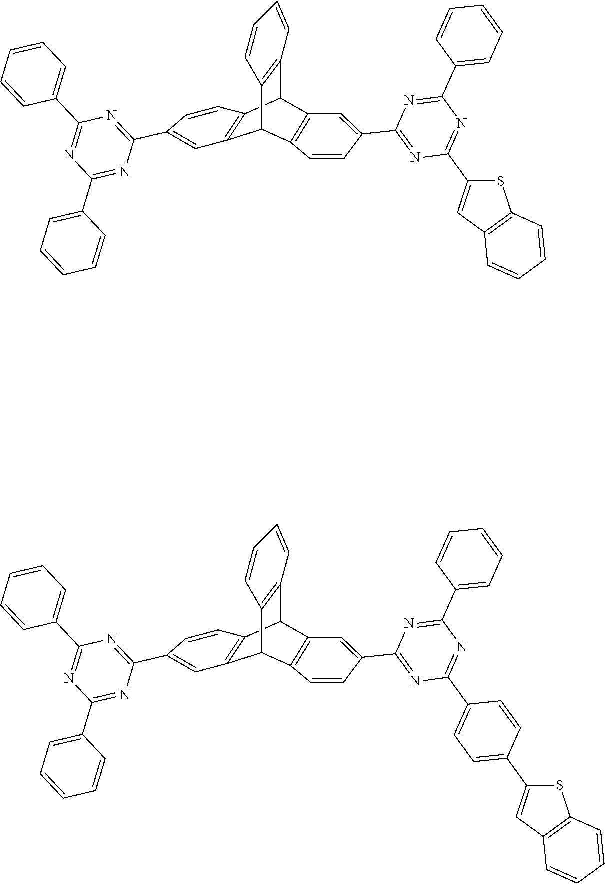 [SMILES: c1ccc(-c2nc(-c3ccccc3)nc(-c3ccc4c(c3)C3c5ccccc5C4c4cc(-c5nc(-c6ccccc6)nc(-c6cc7ccccc7s6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cc8ccccc8s7)cc6)n5)ccc43)n2)cc1]

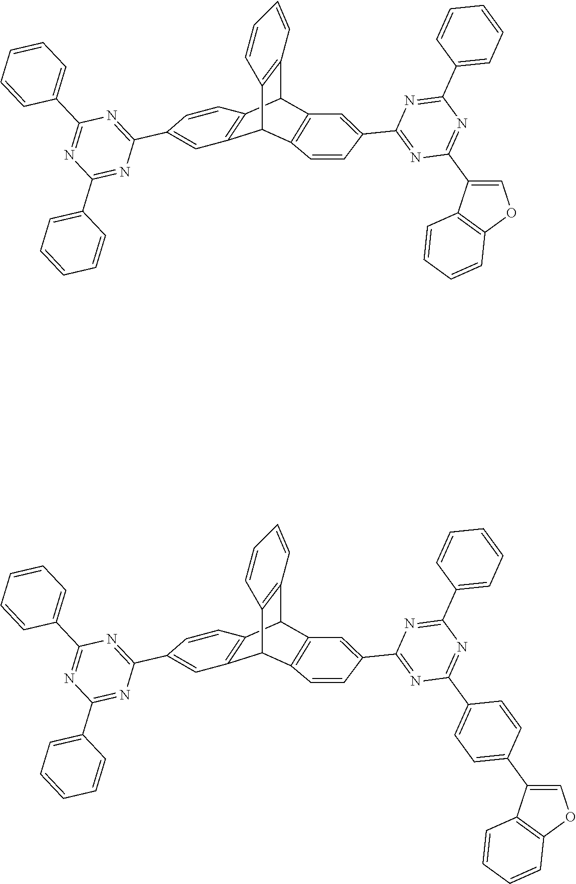 [SMILES: c1ccc(-c2nc(-c3ccccc3)nc(-c3ccc4c(c3)C3c5ccccc5C4c4cc(-c5nc(-c6ccccc6)nc(-c6coc7ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7coc8ccccc78)cc6)n5)ccc43)n2)cc1]

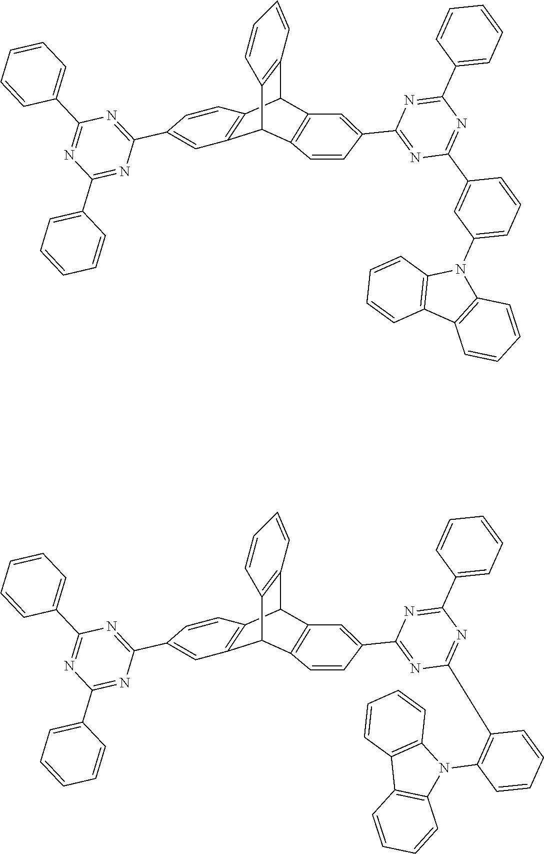 [SMILES: c1ccc(-c2nc(-c3ccccc3)nc(-c3ccc4c(c3)C3c5ccccc5C4c4cc(-c5nc(-c6ccccc6)nc(-c6cccc(-n7c8ccccc8c8ccccc87)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-n6c7ccccc7c7ccccc76)n5)ccc43)n2)cc1]

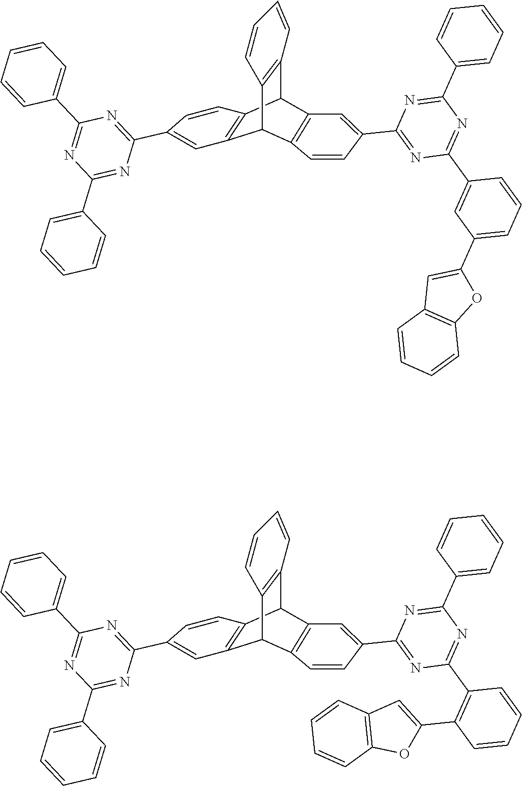 [SMILES: c1ccc(-c2nc(-c3ccccc3)nc(-c3ccc4c(c3)C3c5ccccc5C4c4cc(-c5nc(-c6ccccc6)nc(-c6cccc(-c7cc8ccccc8o7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cc7ccccc7o6)n5)ccc43)n2)cc1]

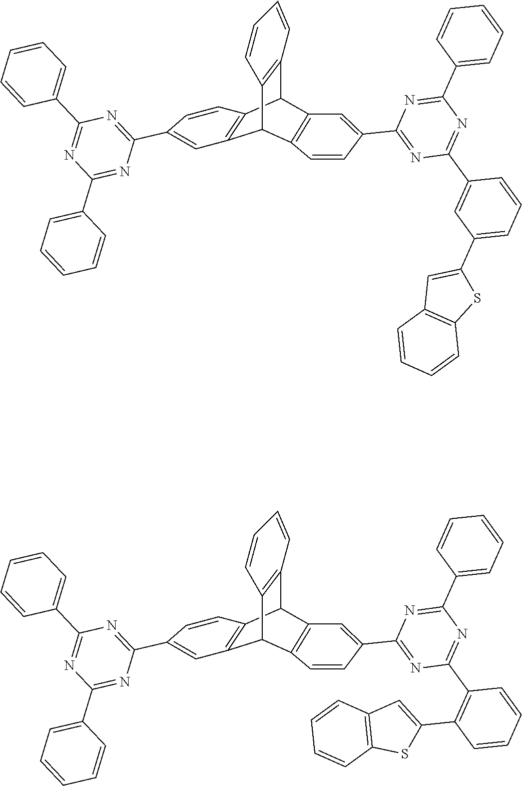 [SMILES: c1ccc(-c2nc(-c3ccccc3)nc(-c3ccc4c(c3)C3c5ccccc5C4c4cc(-c5nc(-c6ccccc6)nc(-c6cccc(-c7cc8ccccc8s7)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cc7ccccc7s6)n5)ccc43)n2)cc1]

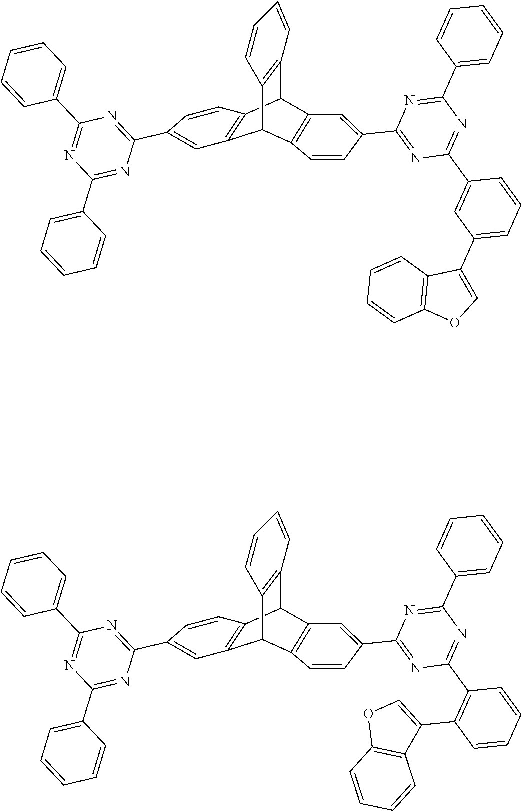 [SMILES: c1ccc(-c2nc(-c3ccccc3)nc(-c3ccc4c(c3)C3c5ccccc5C4c4cc(-c5nc(-c6ccccc6)nc(-c6cccc(-c7coc8ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6coc7ccccc67)n5)ccc43)n2)cc1]

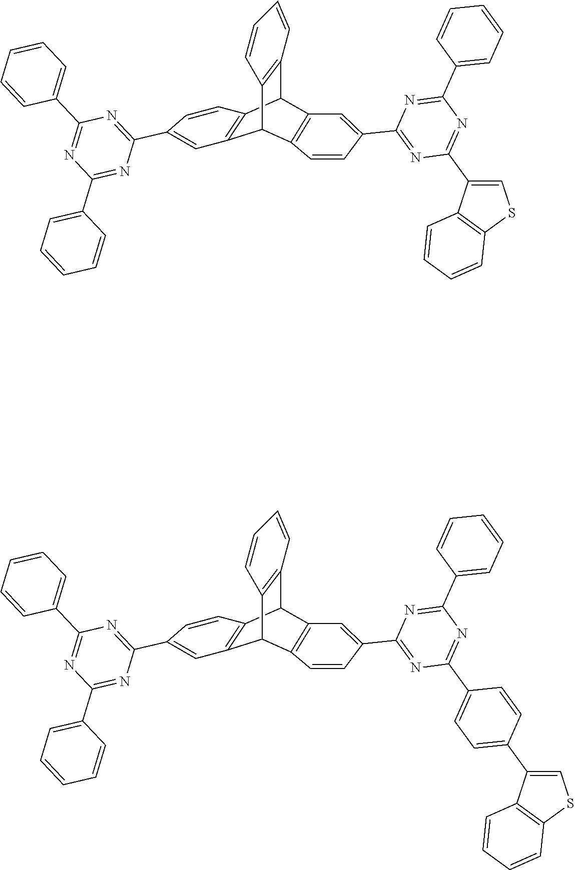 [SMILES: c1ccc(-c2nc(-c3ccccc3)nc(-c3ccc4c(c3)C3c5ccccc5C4c4cc(-c5nc(-c6ccccc6)nc(-c6csc7ccccc67)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7csc8ccccc78)cc6)n5)ccc43)n2)cc1]

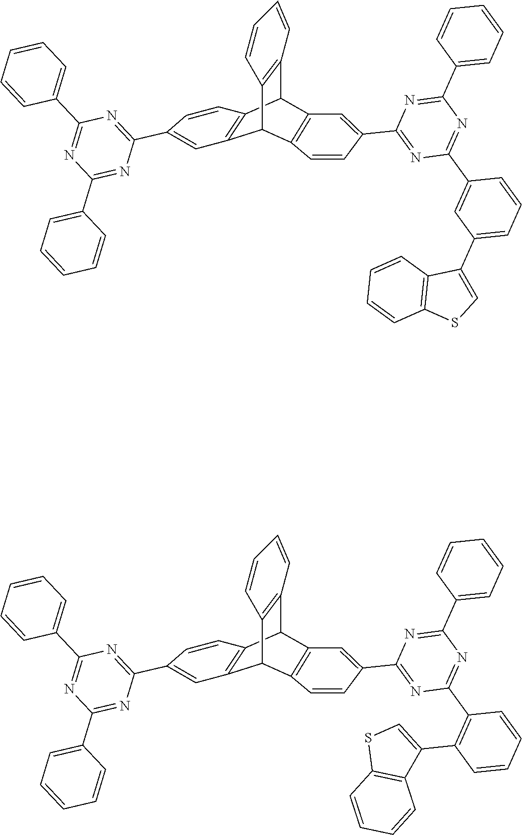 [SMILES: c1ccc(-c2nc(-c3ccccc3)nc(-c3ccc4c(c3)C3c5ccccc5C4c4cc(-c5nc(-c6ccccc6)nc(-c6cccc(-c7csc8ccccc78)c6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6csc7ccccc67)n5)ccc43)n2)cc1]

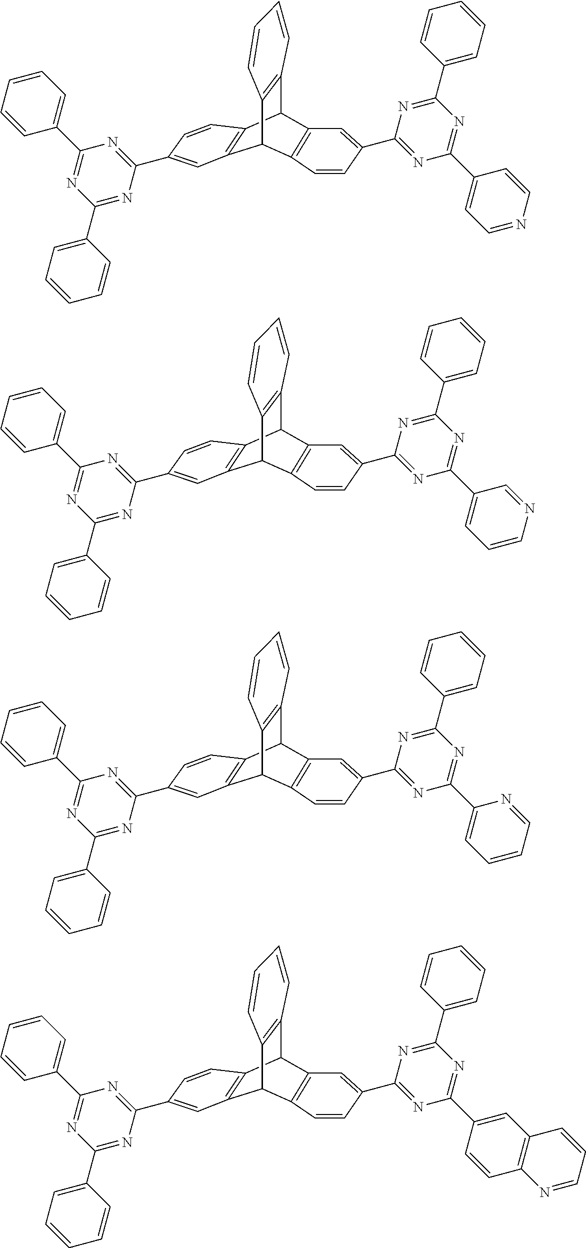 [SMILES: c1ccc(-c2nc(-c3ccccc3)nc(-c3ccc4c(c3)C3c5ccccc5C4c4cc(-c5nc(-c6ccccc6)nc(-c6ccncc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6cccnc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccn6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc7ncccc7c6)n5)ccc43)n2)cc1]

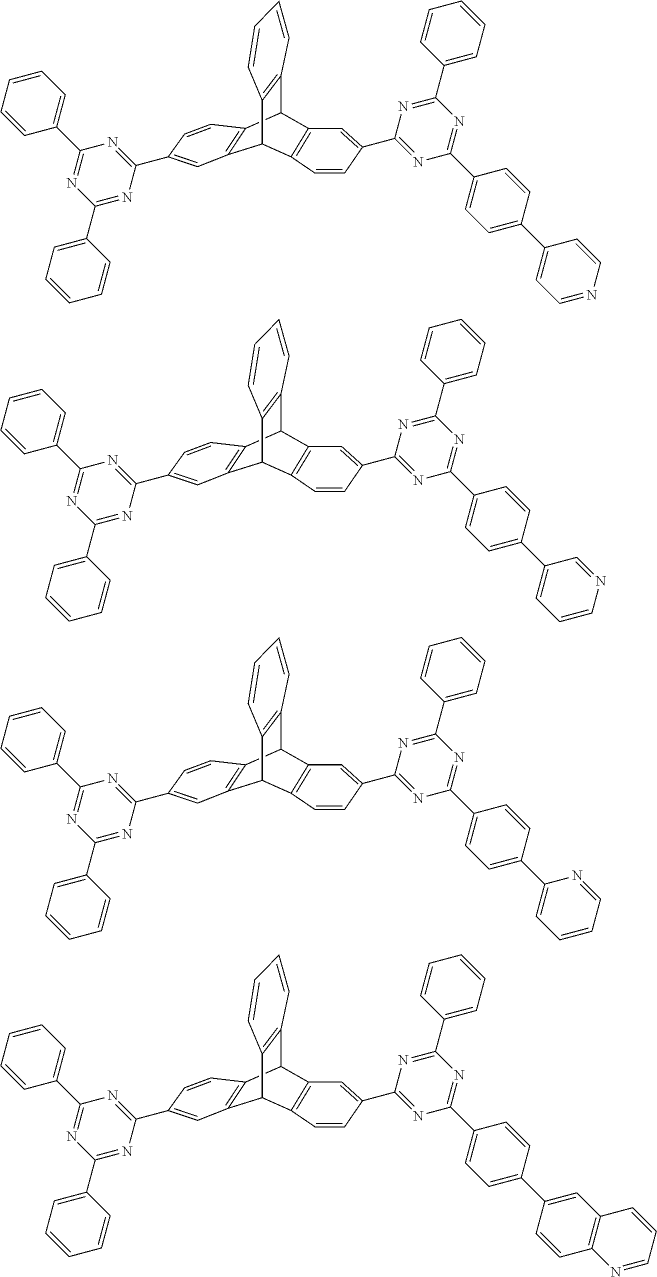 [SMILES: c1ccc(-c2nc(-c3ccccc3)nc(-c3ccc4c(c3)C3c5ccccc5C4c4cc(-c5nc(-c6ccccc6)nc(-c6ccc(-c7ccncc7)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7cccnc7)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccccn7)cc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccc(-c7ccc8ncccc8c7)cc6)n5)ccc43)n2)cc1]

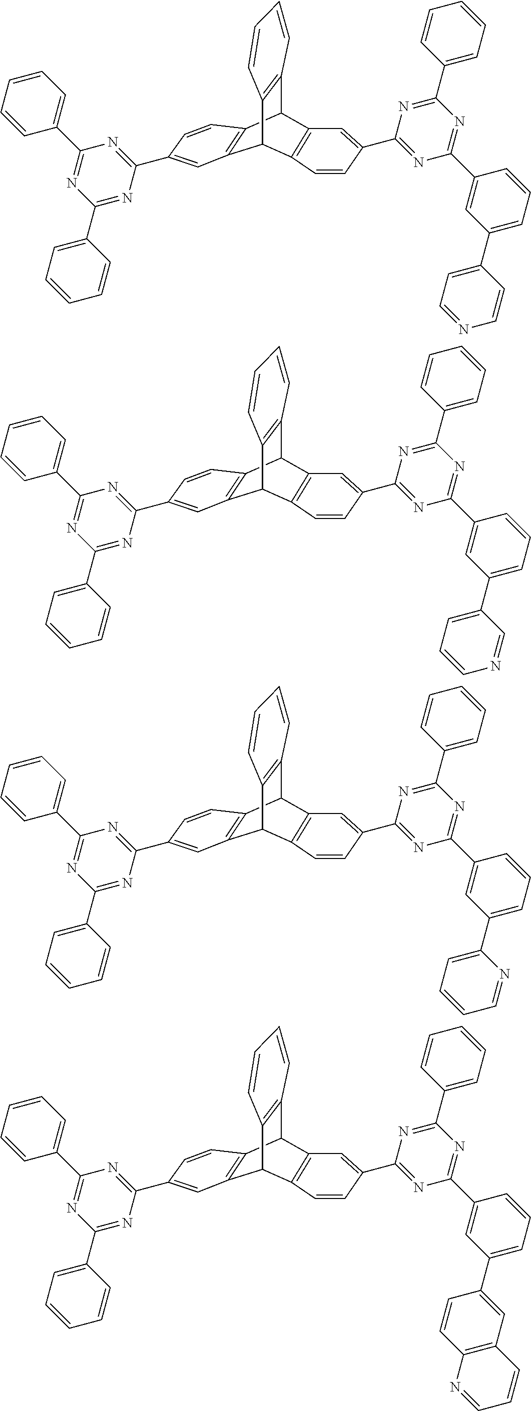

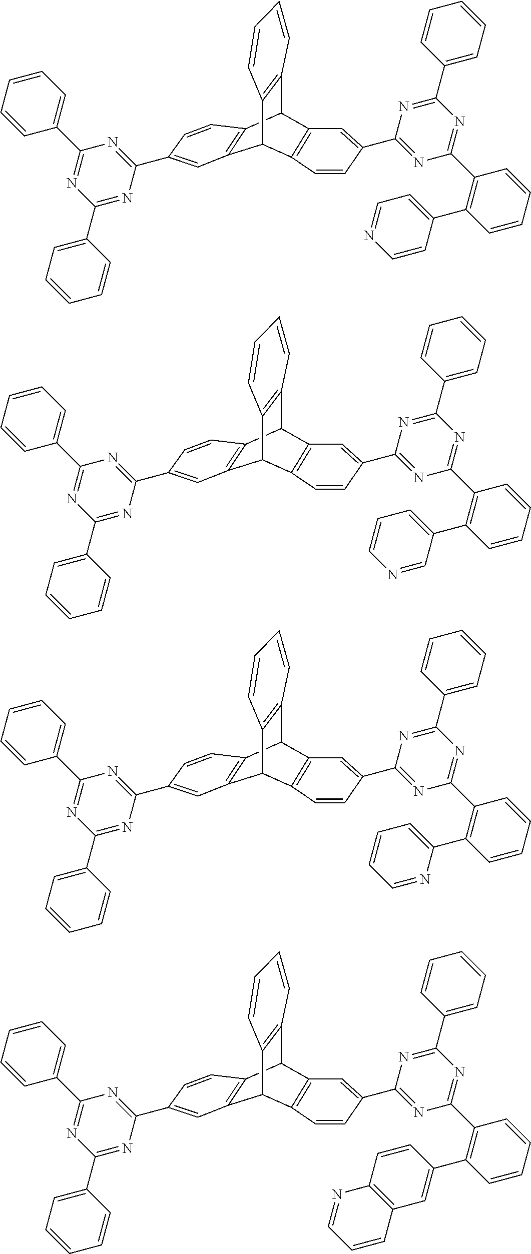 [SMILES: c1ccc(-c2nc(-c3ccccc3)nc(-c3ccc4c(c3)C3c5ccccc5C4c4cc(-c5nc(-c6ccccc6)nc(-c6ccccc6-c6ccncc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6cccnc6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccccn6)n5)ccc43)n2)cc1 ; c1ccc(-c2nc(-c3ccccc3)nc(-c3ccc4c(c3)C3c5ccccc5C4c4cc(-c5nc(-c6ccccc6)nc(-c6ccccc6-c6ccc7ncccc7c6)n5)ccc43)n2)cc1]

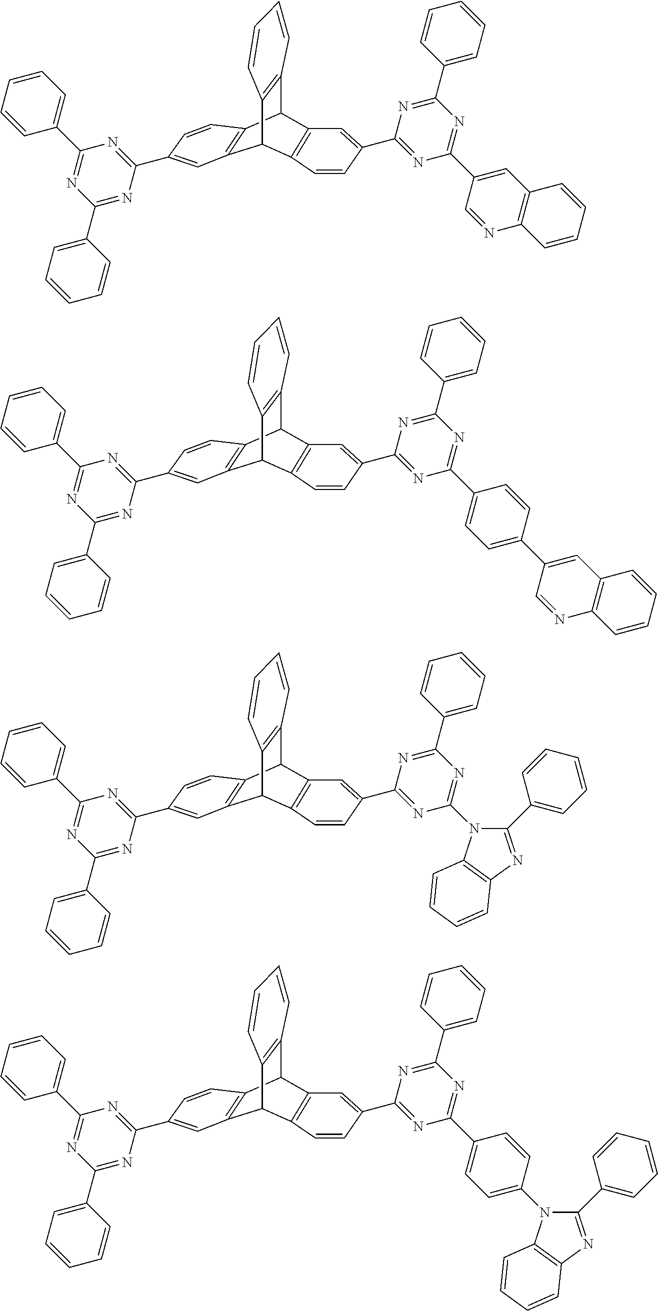

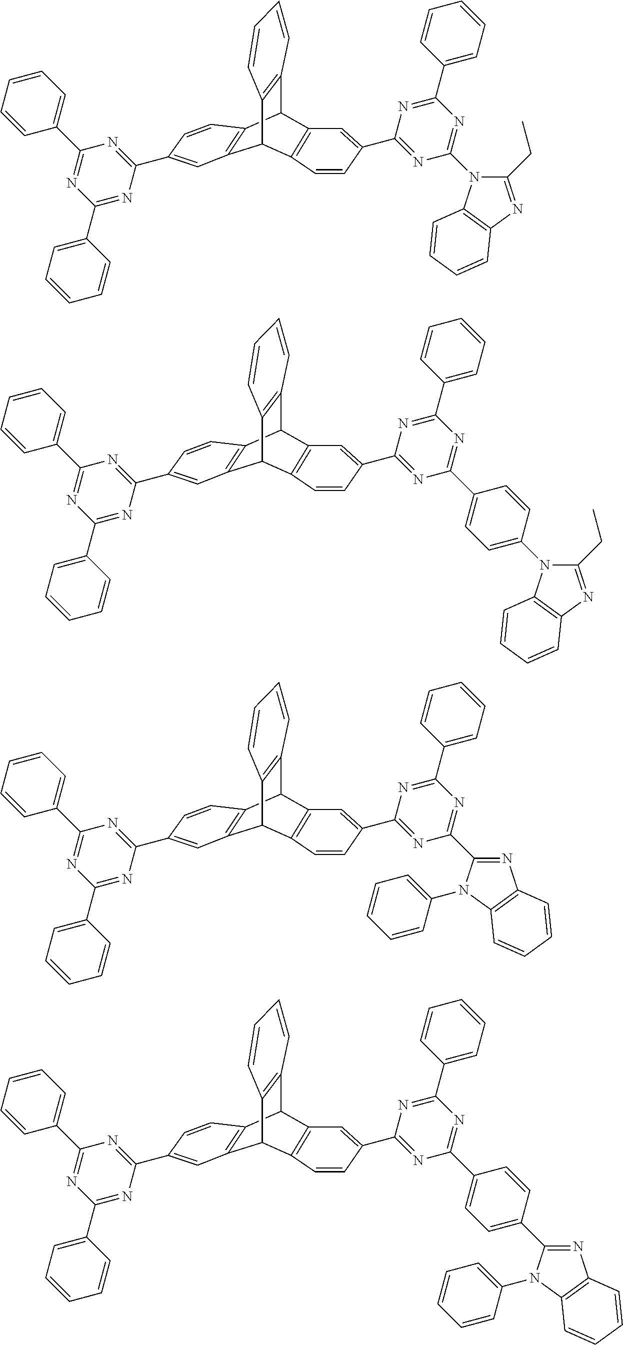

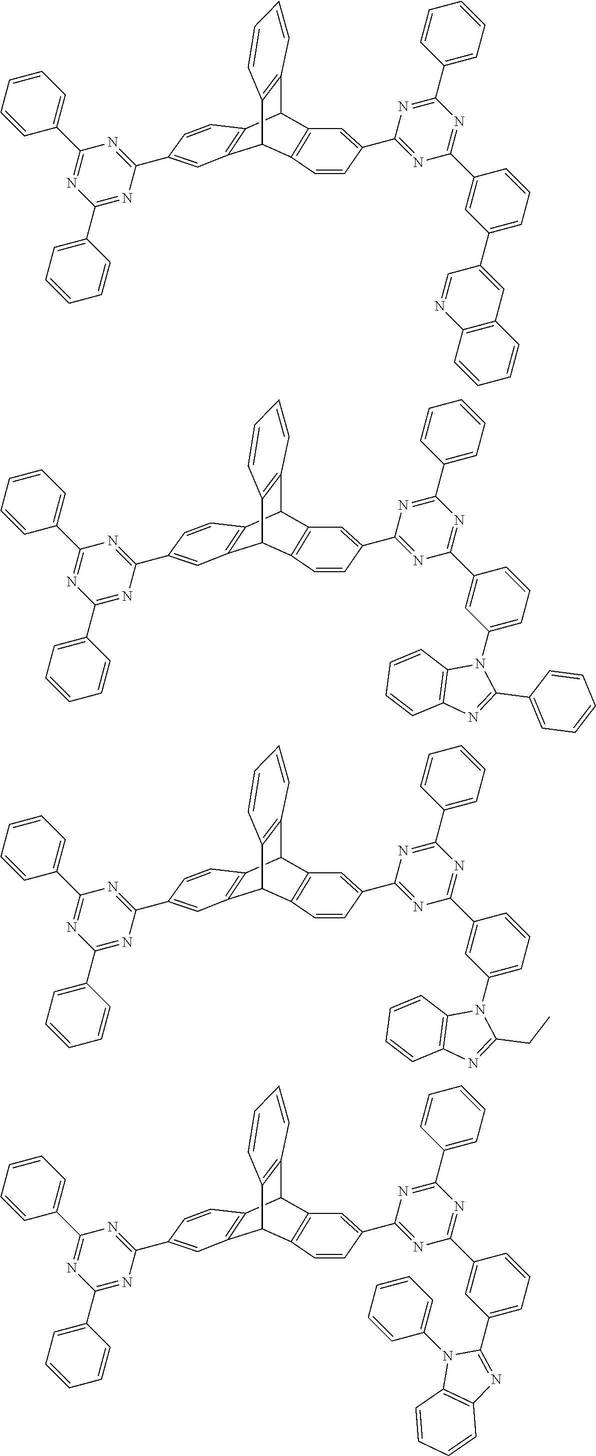

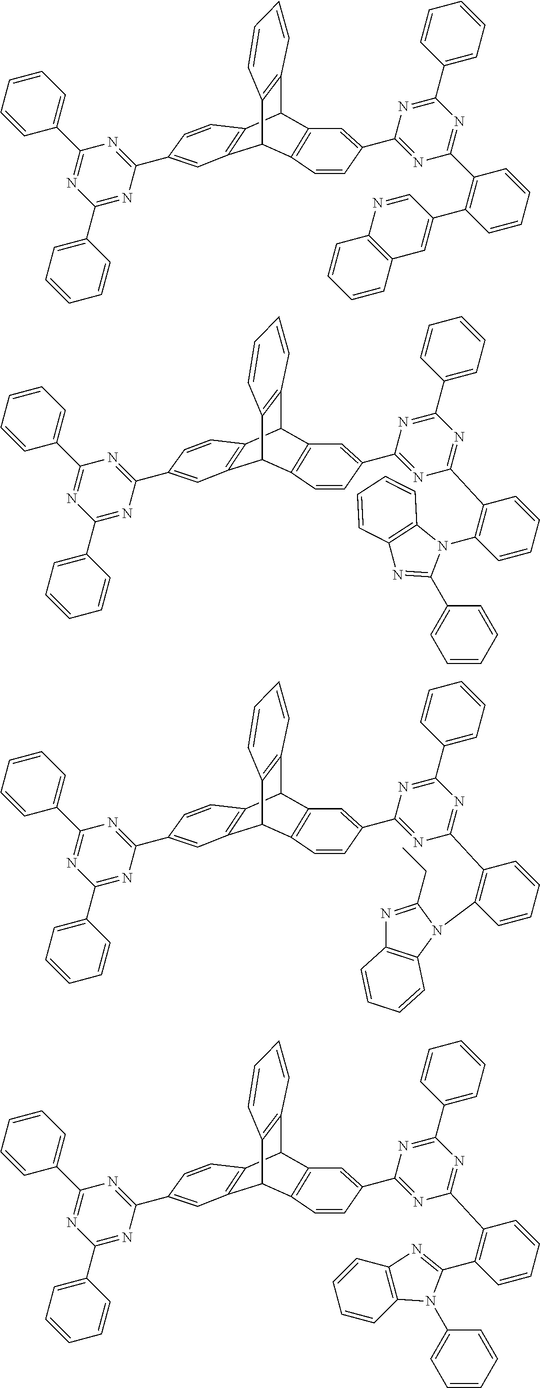

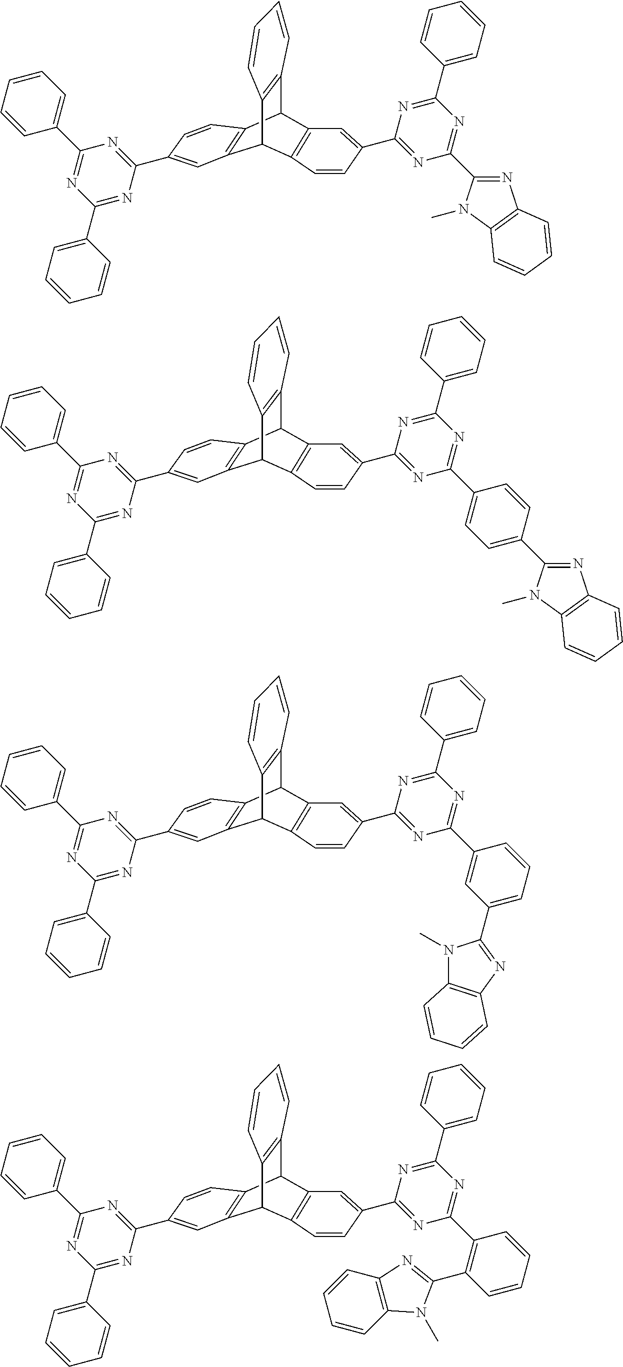

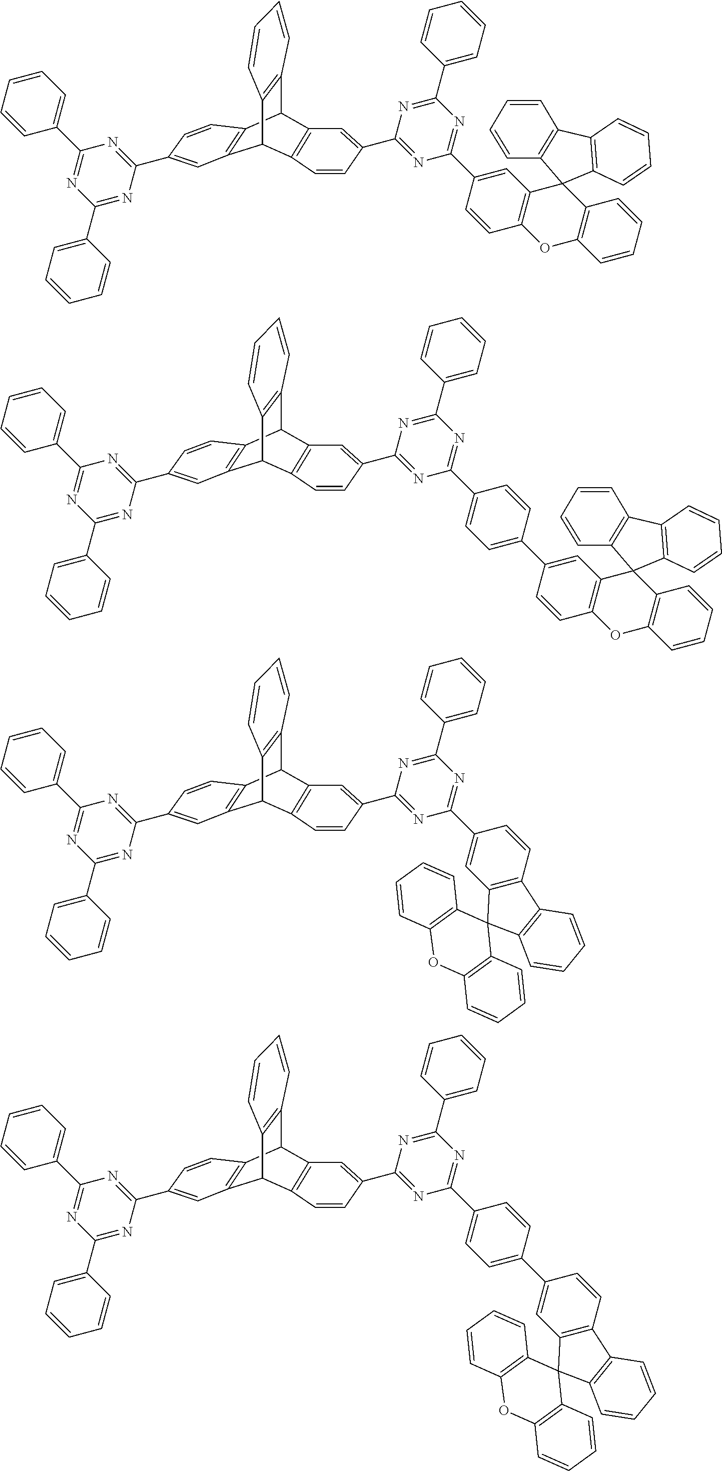

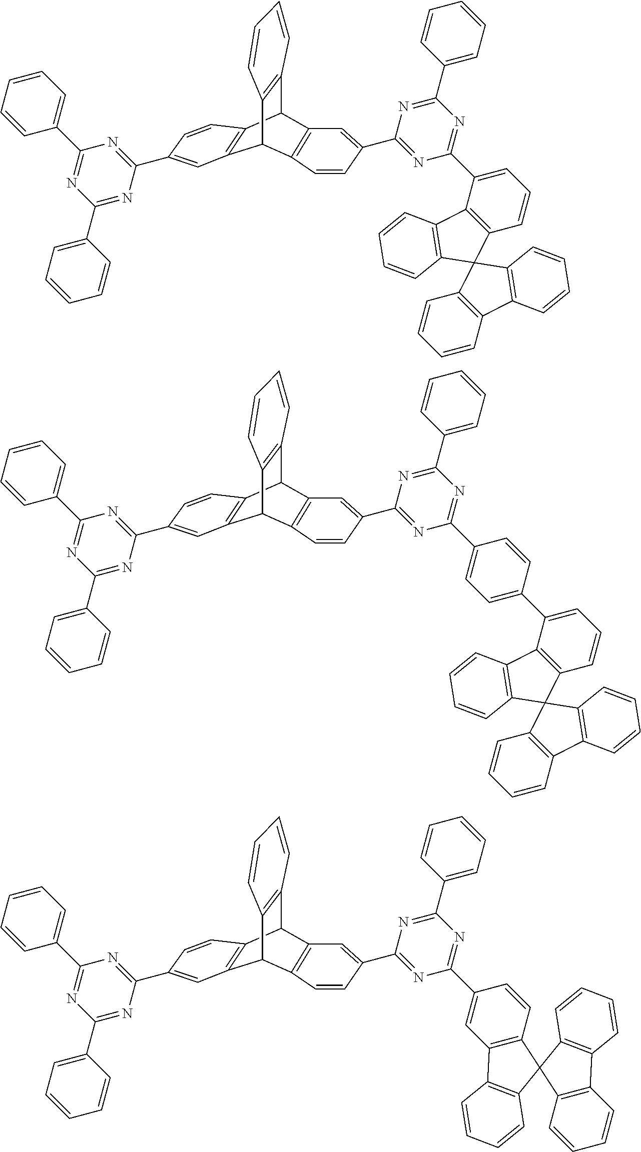

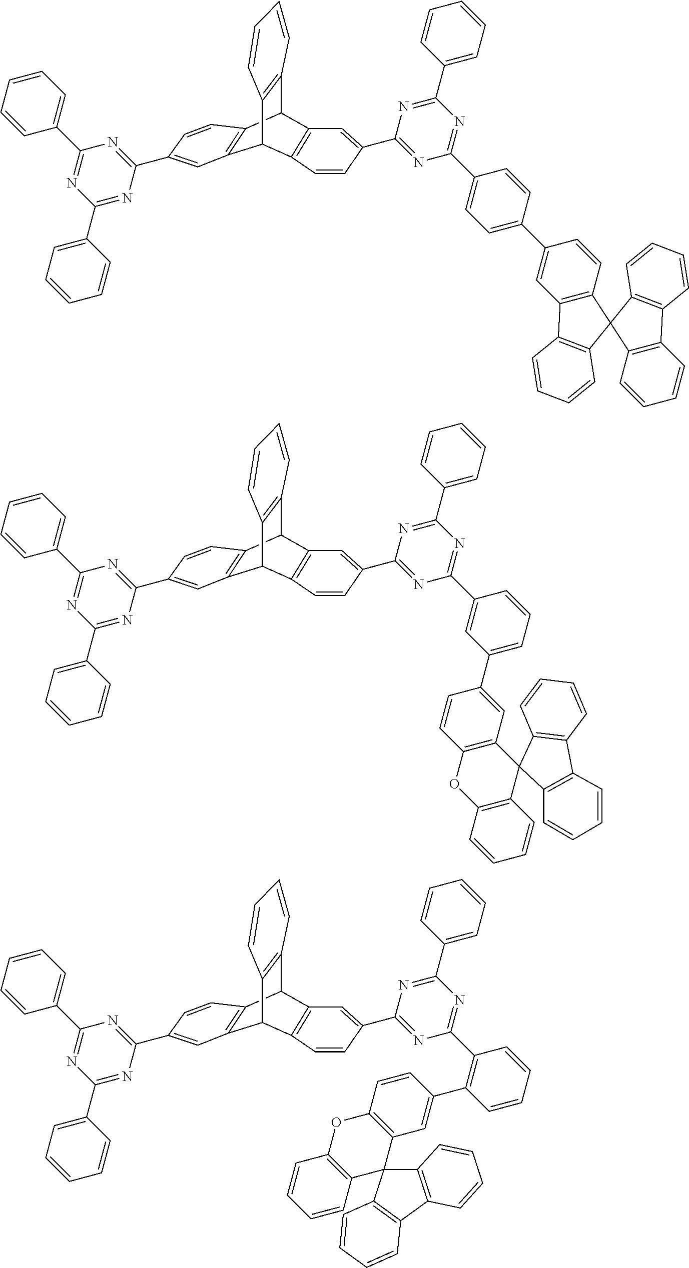

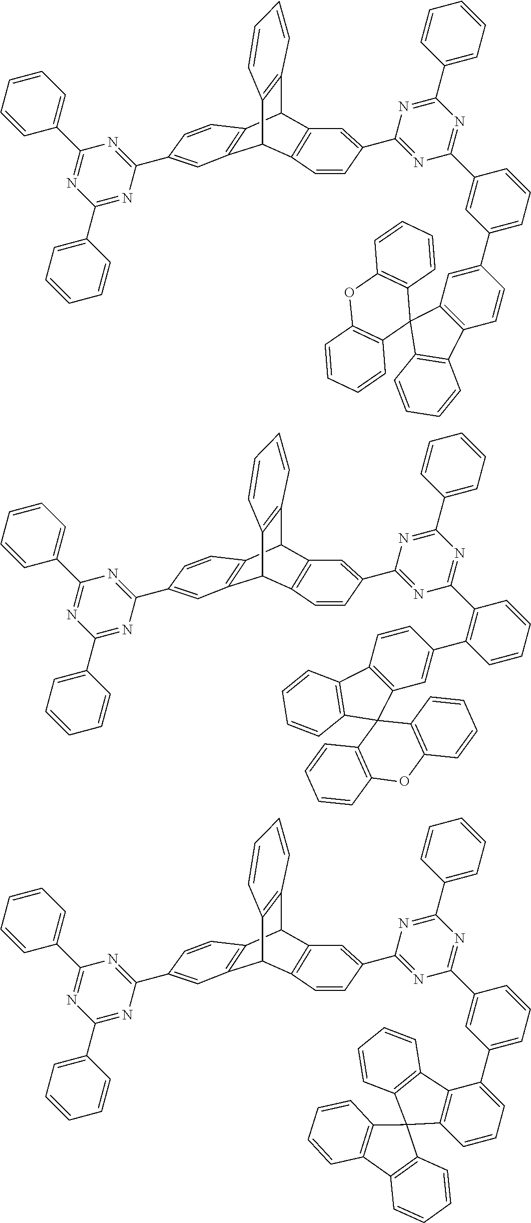

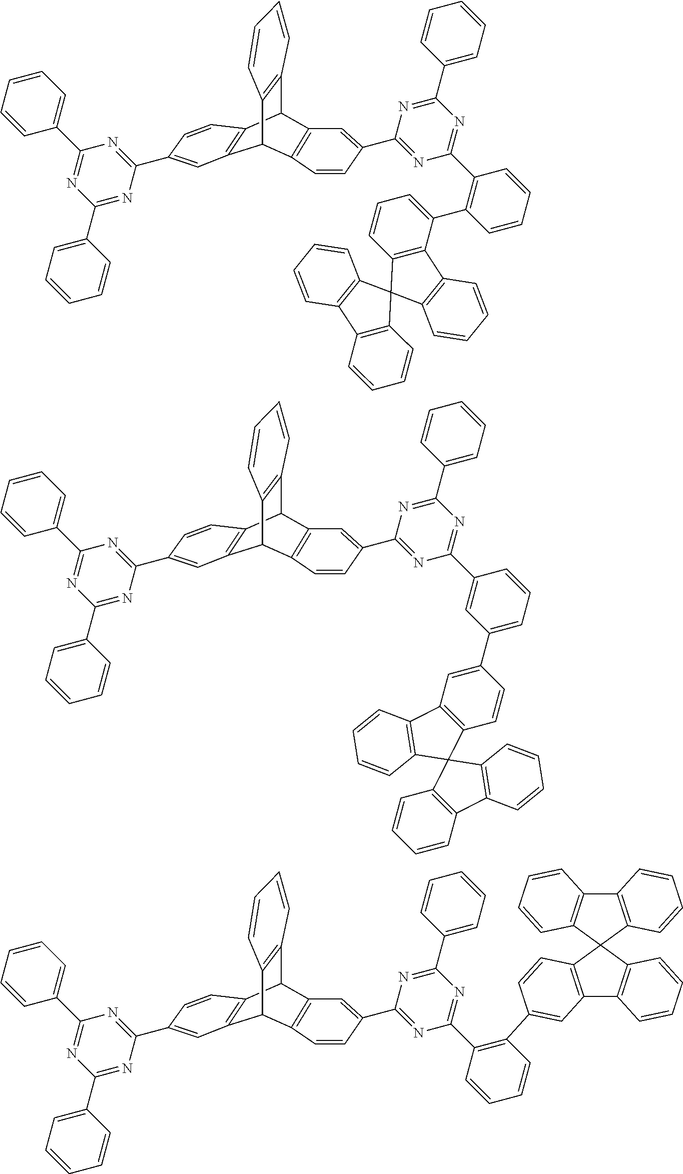

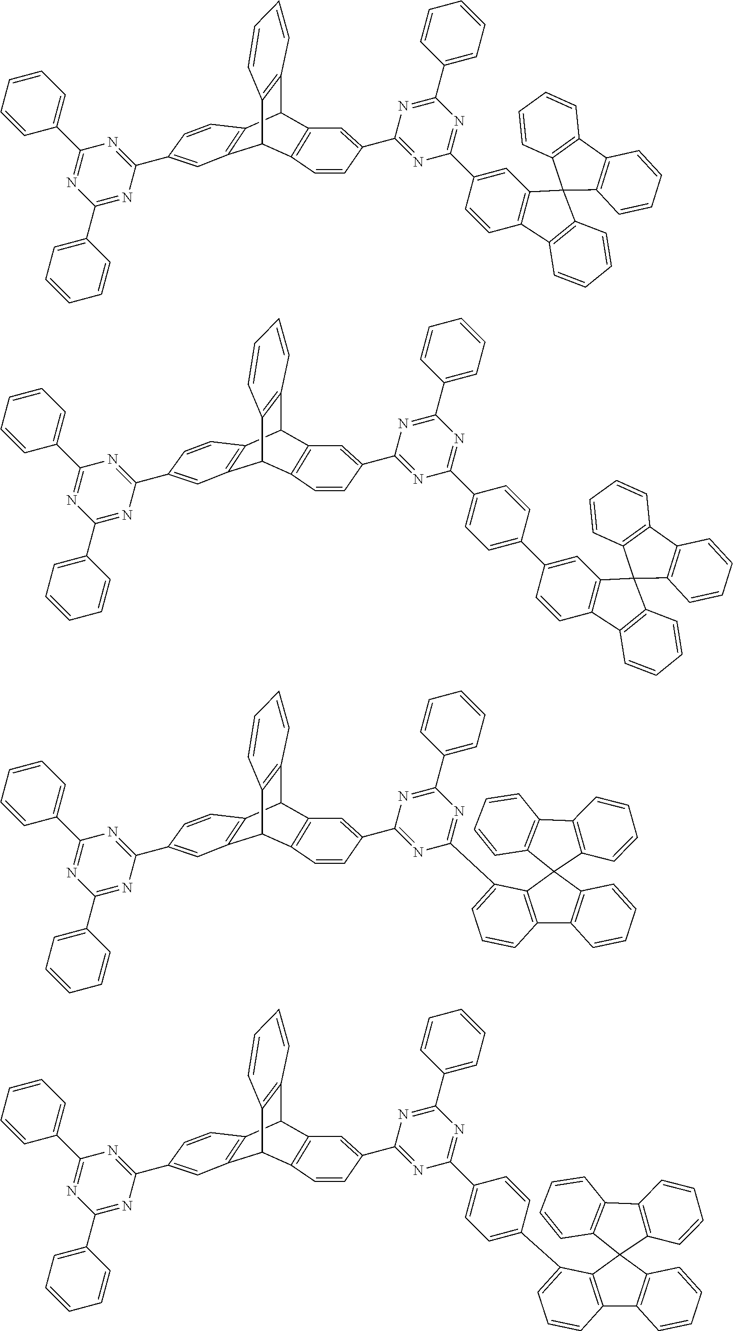

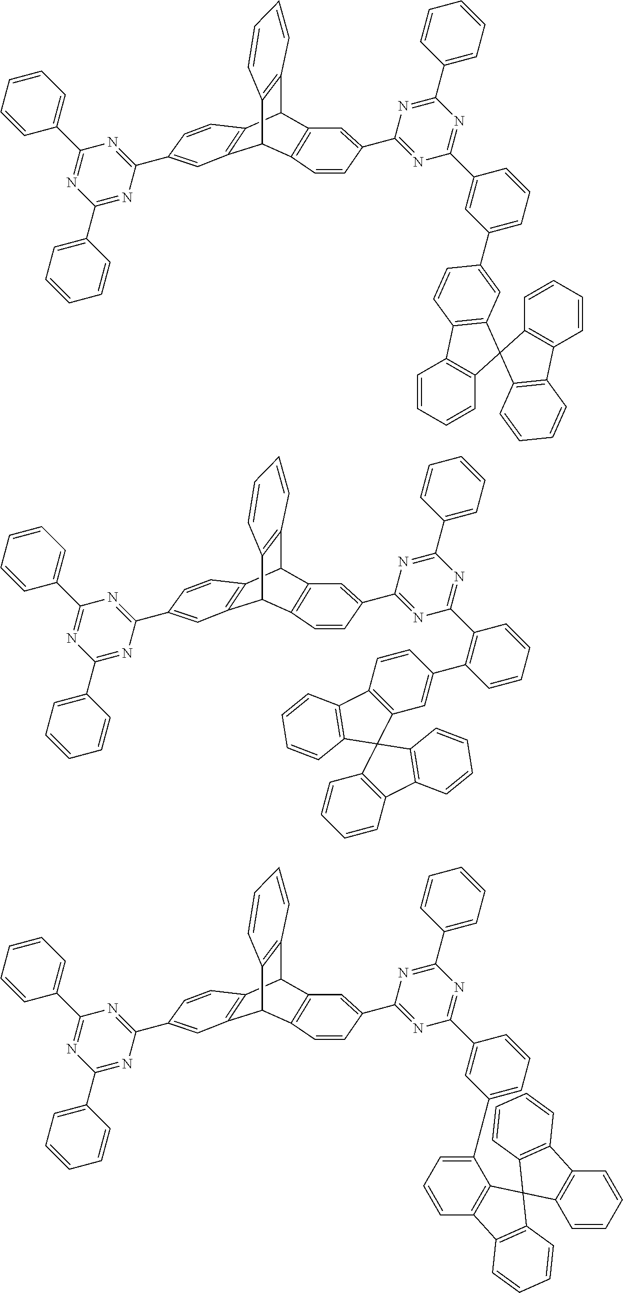

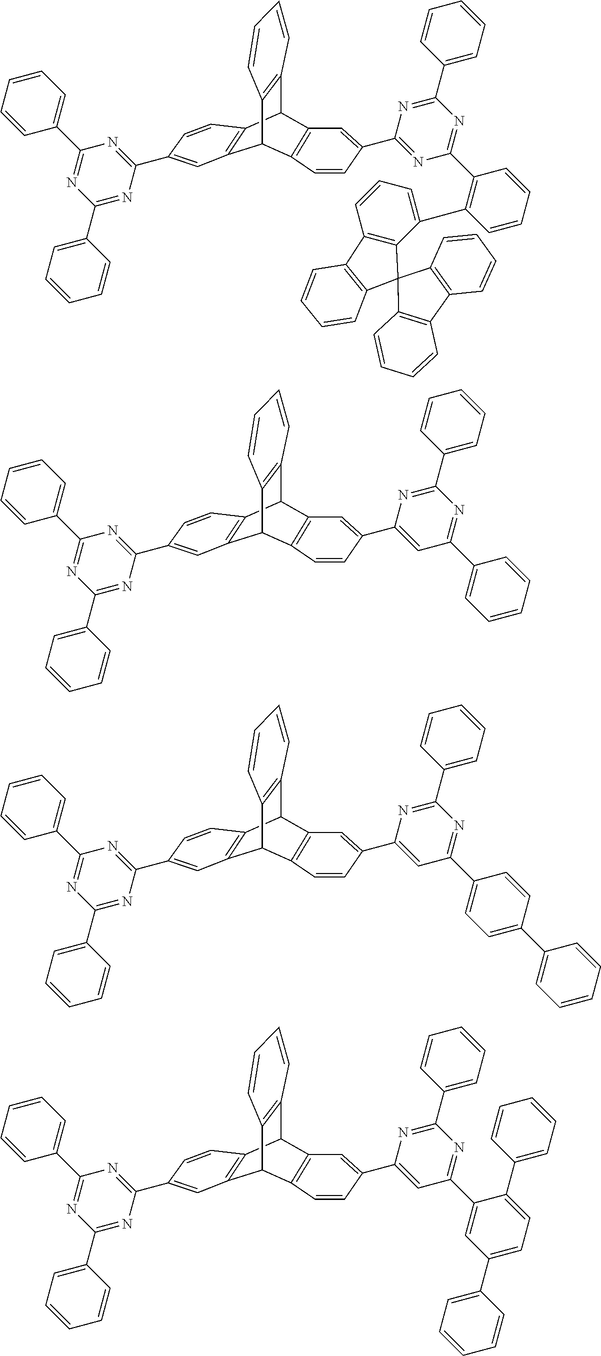

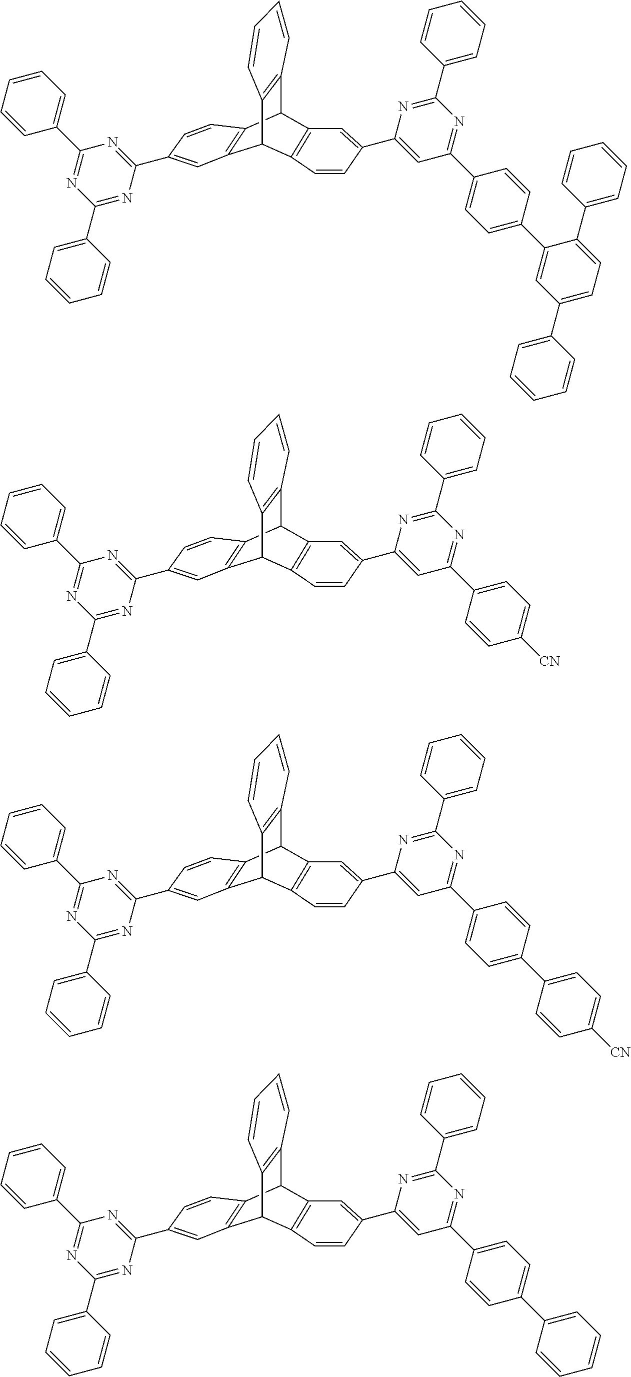 [SMILES: c1ccc(-c2ccc(-c3ccccc3)c(-c3ccc(-c4cc(-c5ccc6c(c5)C5c7ccccc7C6c6cc(-c7nc(-c8ccccc8)nc(-c8ccccc8)n7)ccc65)nc(-c5ccccc5)n4)cc3)c2)cc1 ; N#Cc1ccc(-c2cc(-c3ccc4c(c3)C3c5ccccc5C4c4cc(-c5nc(-c6ccccc6)nc(-c6ccccc6)n5)ccc43)nc(-c3ccccc3)n2)cc1 ; N#Cc1ccc(-c2ccc(-c3cc(-c4ccc5c(c4)C4c6ccccc6C5c5cc(-c6nc(-c7ccccc7)nc(-c7ccccc7)n6)ccc54)nc(-c4ccccc4)n3)cc2)cc1 ; c1ccc(-c2ccc(-c3cc(-c4ccc5c(c4)C4c6ccccc6C5c5cc(-c6nc(-c7ccccc7)nc(-c7ccccc7)n6)ccc54)nc(-c4ccccc4)n3)cc2)cc1]

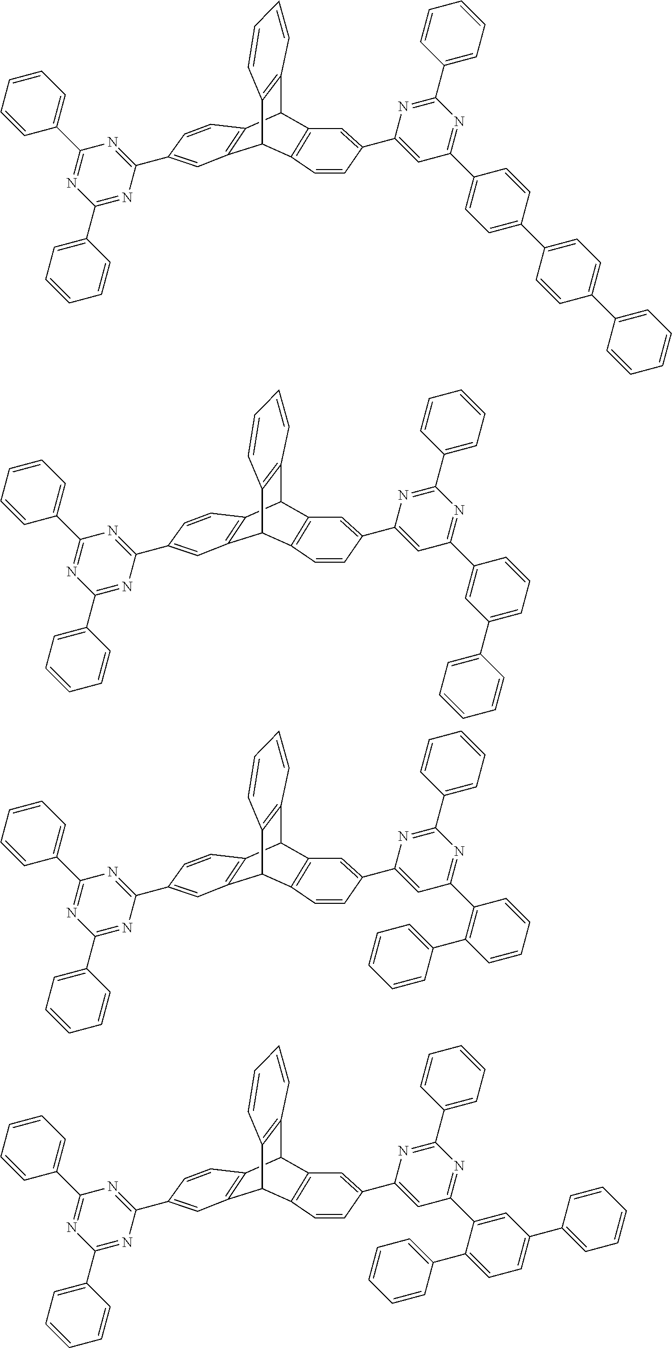

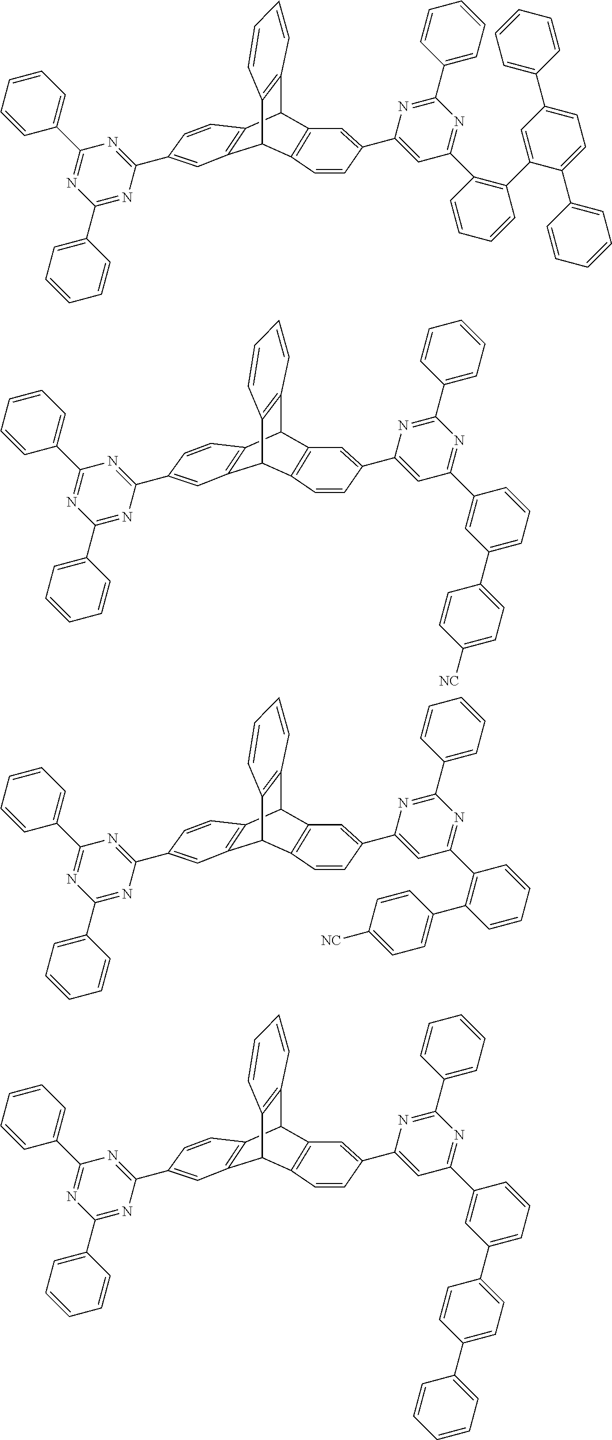

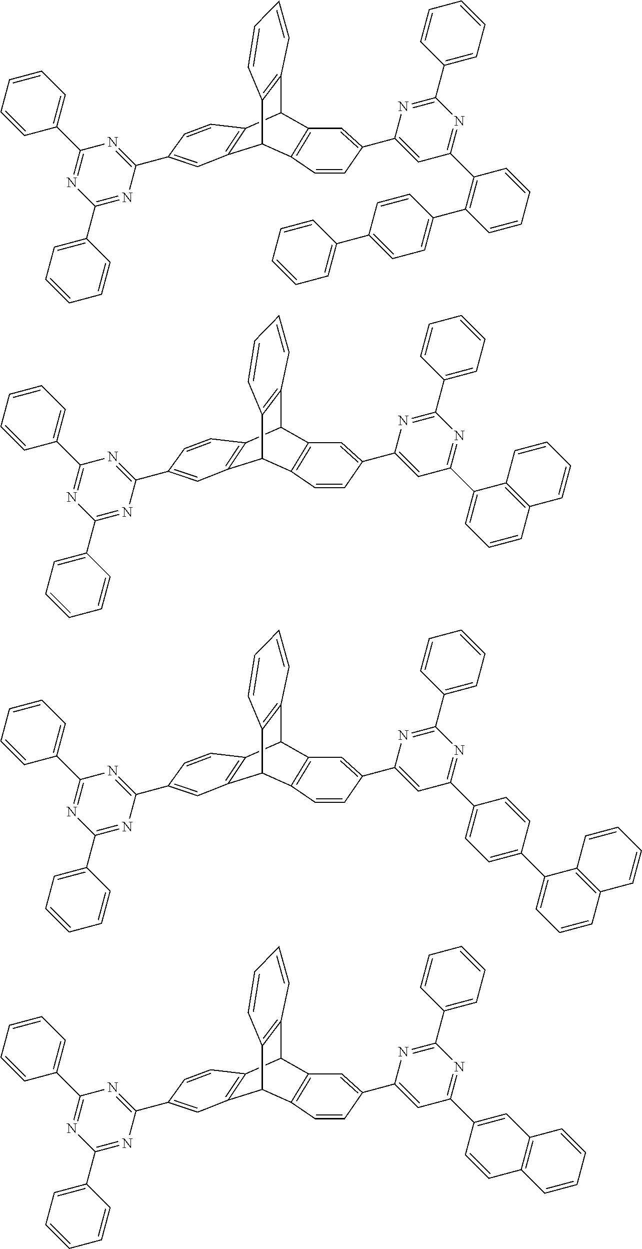

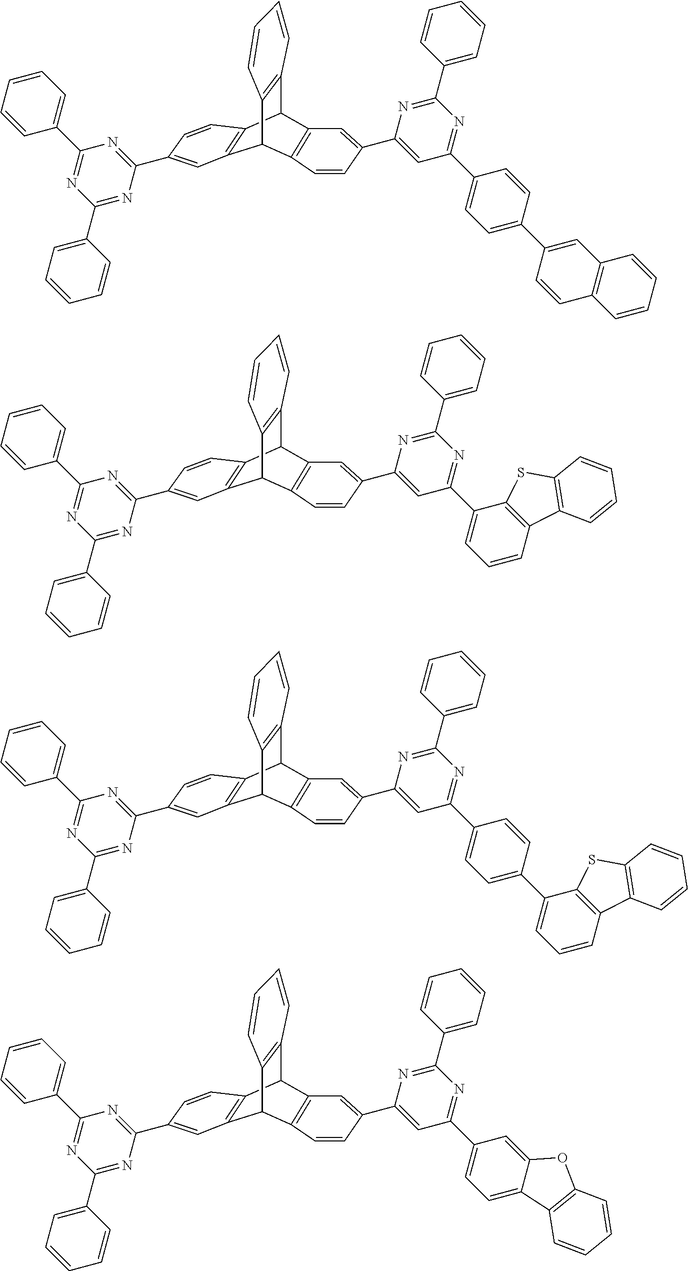 [SMILES: c1ccc(-c2nc(-c3ccc(-c4ccc5ccccc5c4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cccc4c3sc3ccccc34)n2)cc1 ; c1ccc(-c2nc(-c3ccc(-c4cccc5c4sc4ccccc45)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)oc3ccccc34)n2)cc1]

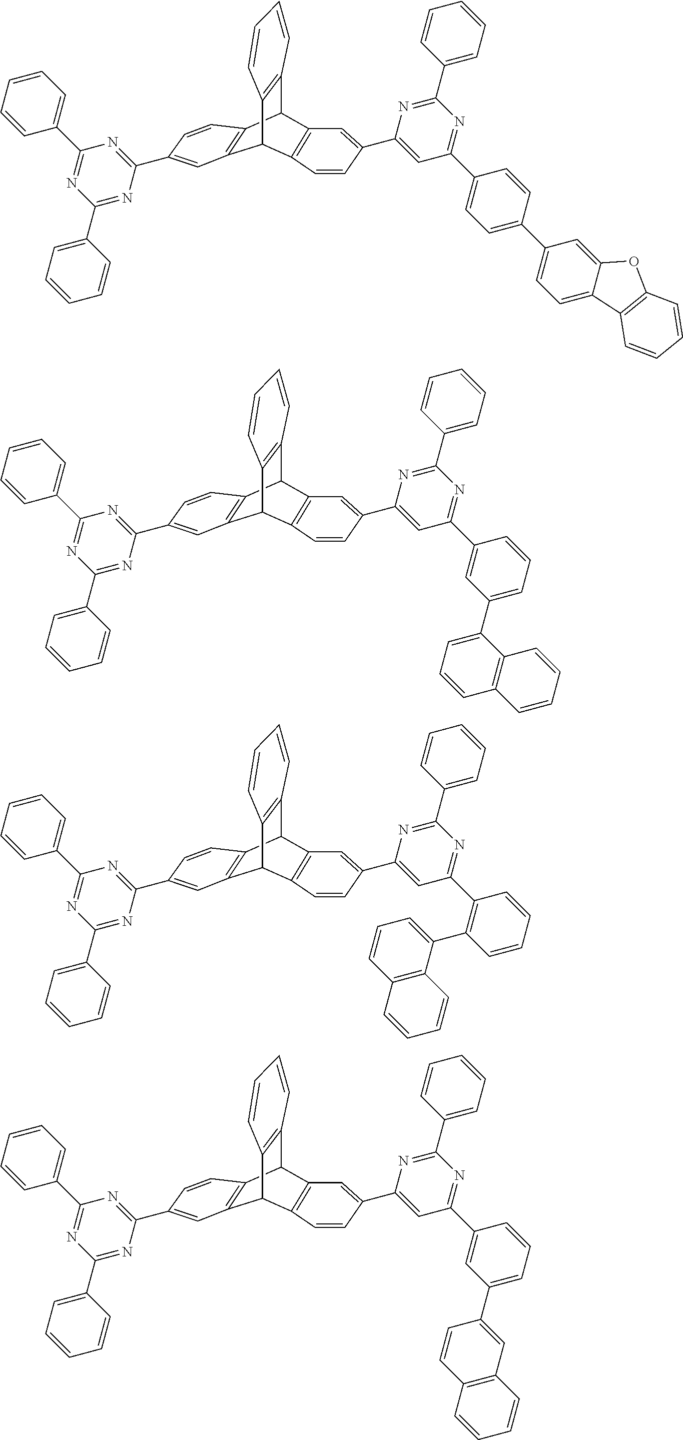

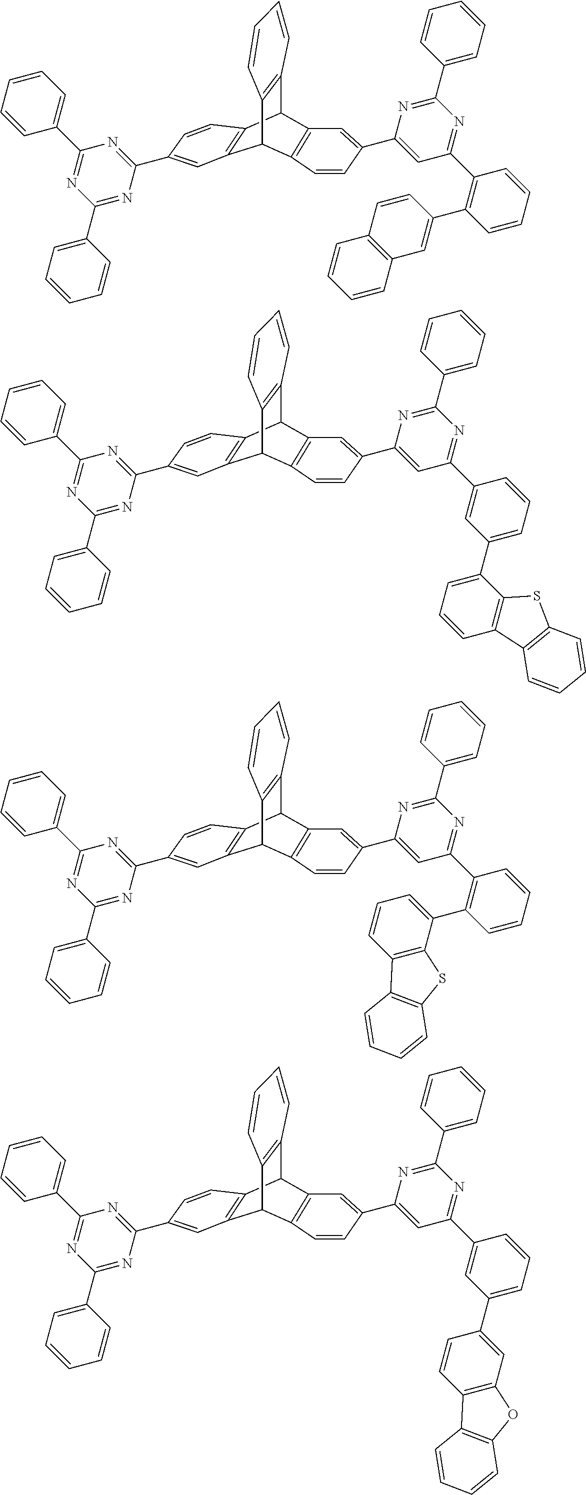

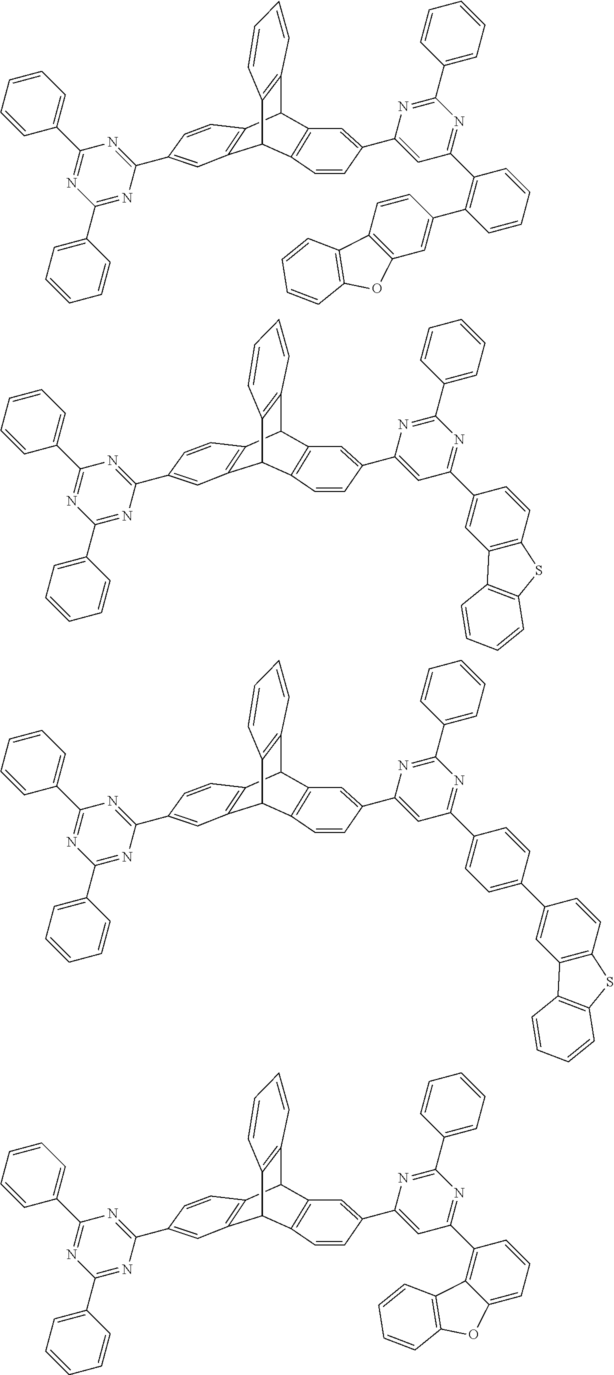

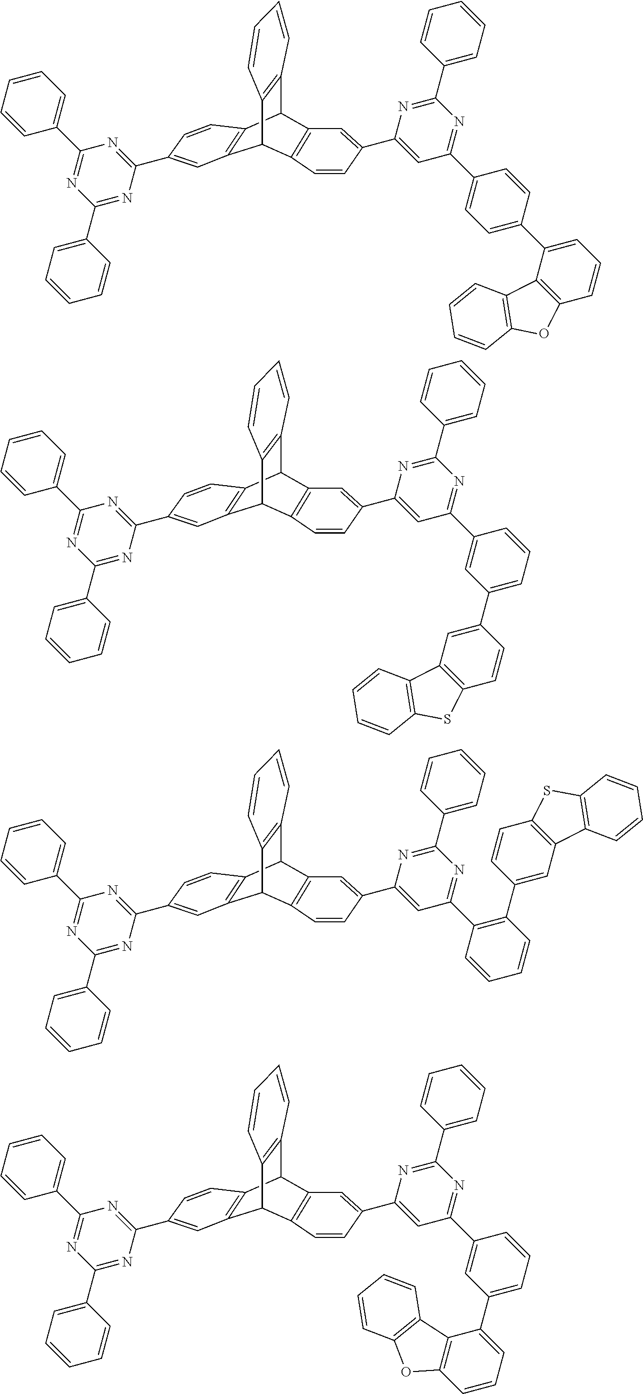

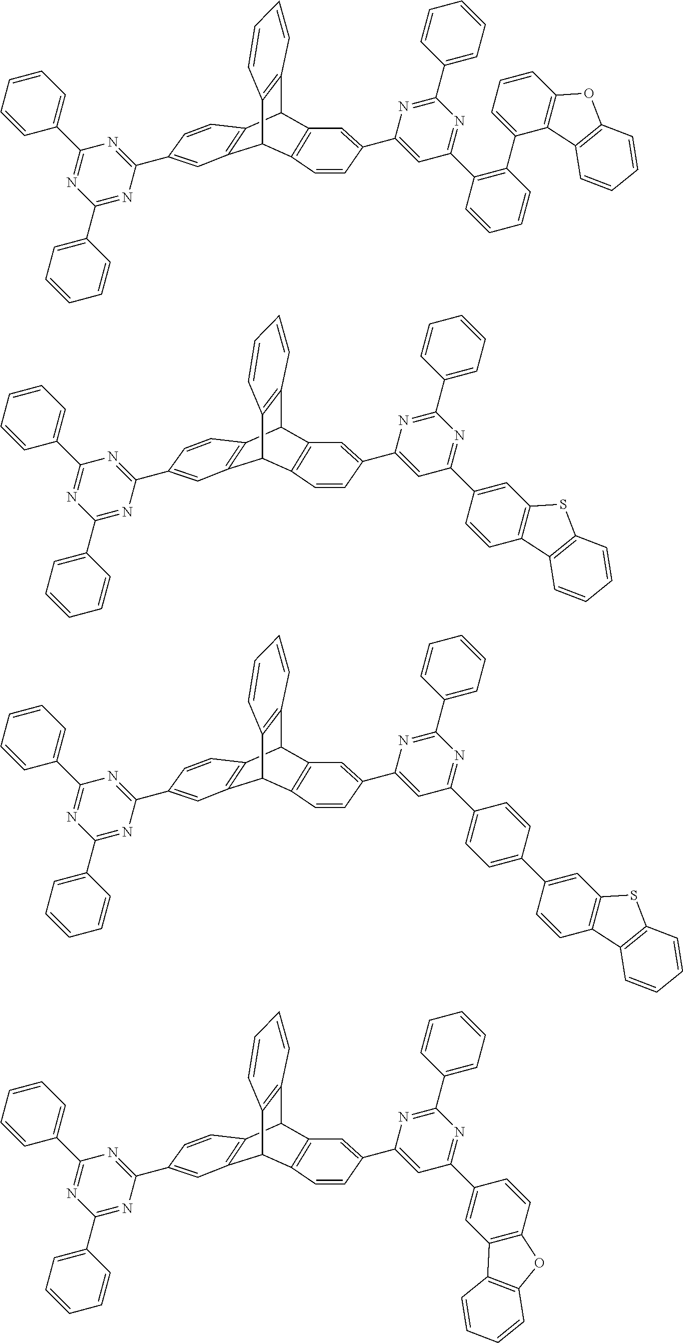 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccc4oc5ccccc5c34)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)sc3ccccc34)n2)cc1 ; c1ccc(-c2nc(-c3ccc(-c4ccc5c(c4)sc4ccccc45)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccc4oc5ccccc5c4c3)n2)cc1]

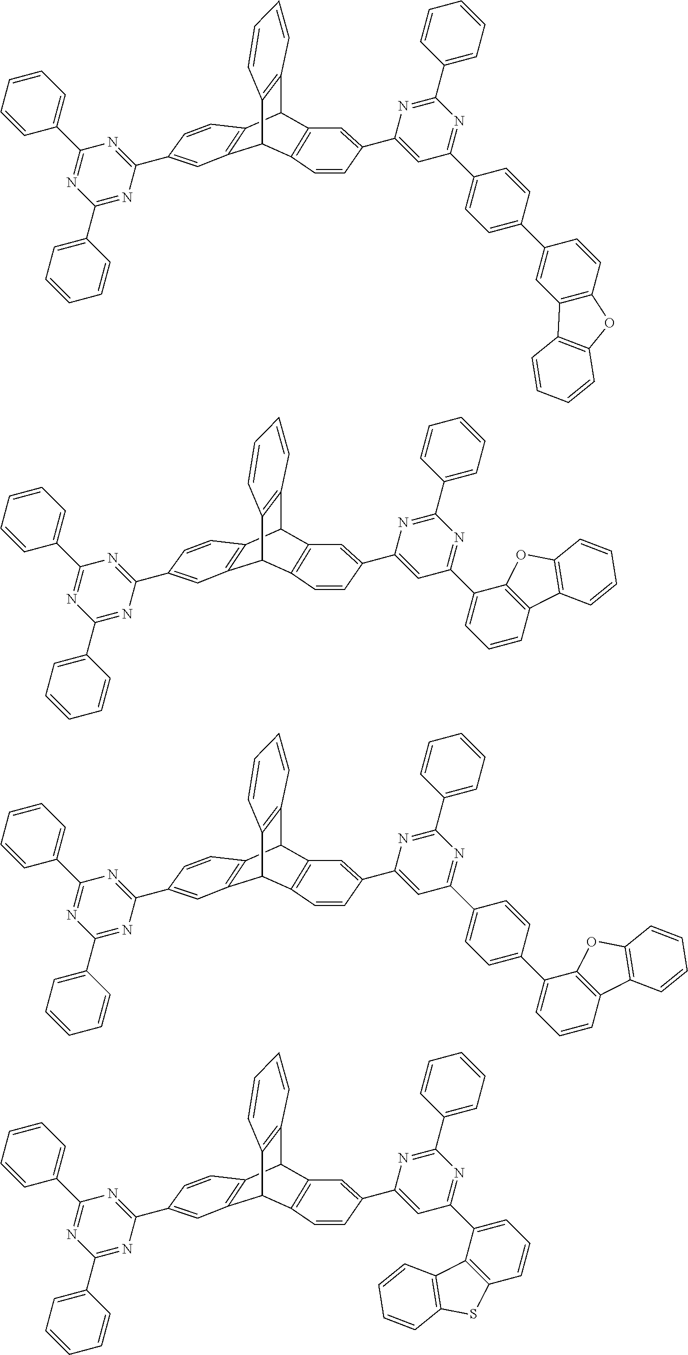 [SMILES: c1ccc(-c2nc(-c3ccc(-c4ccc5oc6ccccc6c5c4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cccc4c3oc3ccccc34)n2)cc1 ; c1ccc(-c2nc(-c3ccc(-c4cccc5c4oc4ccccc45)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cccc4sc5ccccc5c34)n2)cc1]

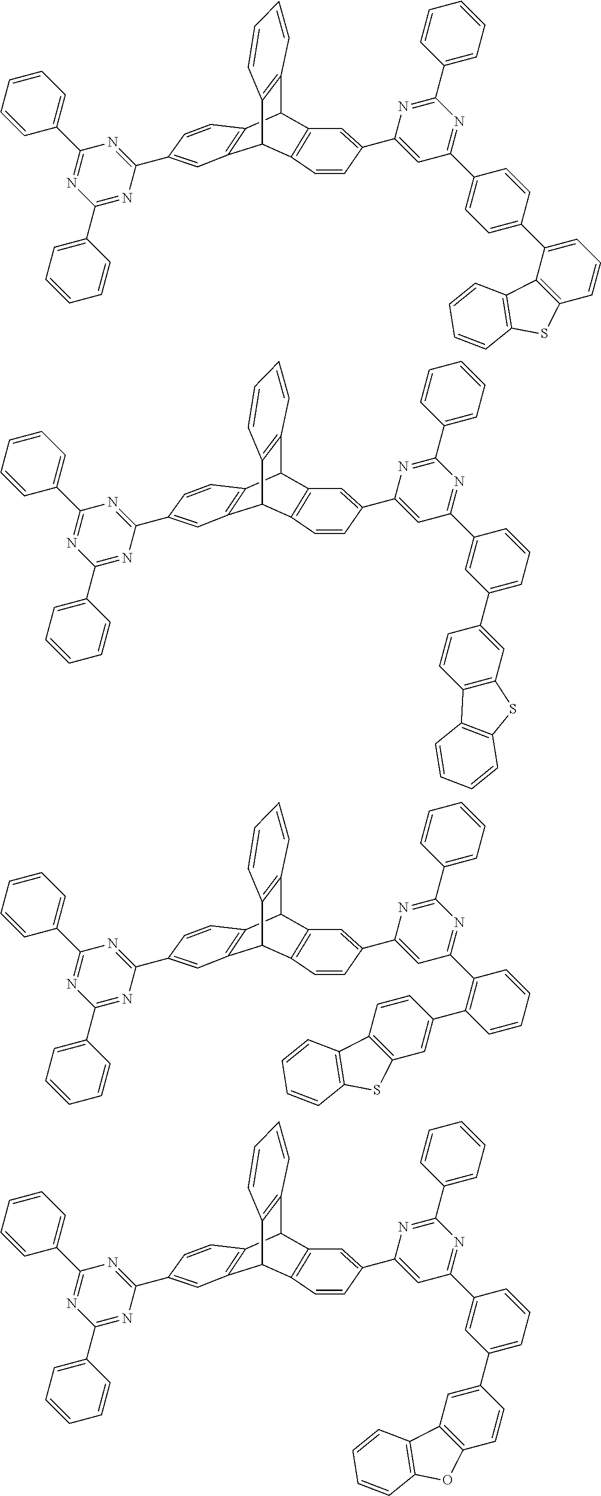

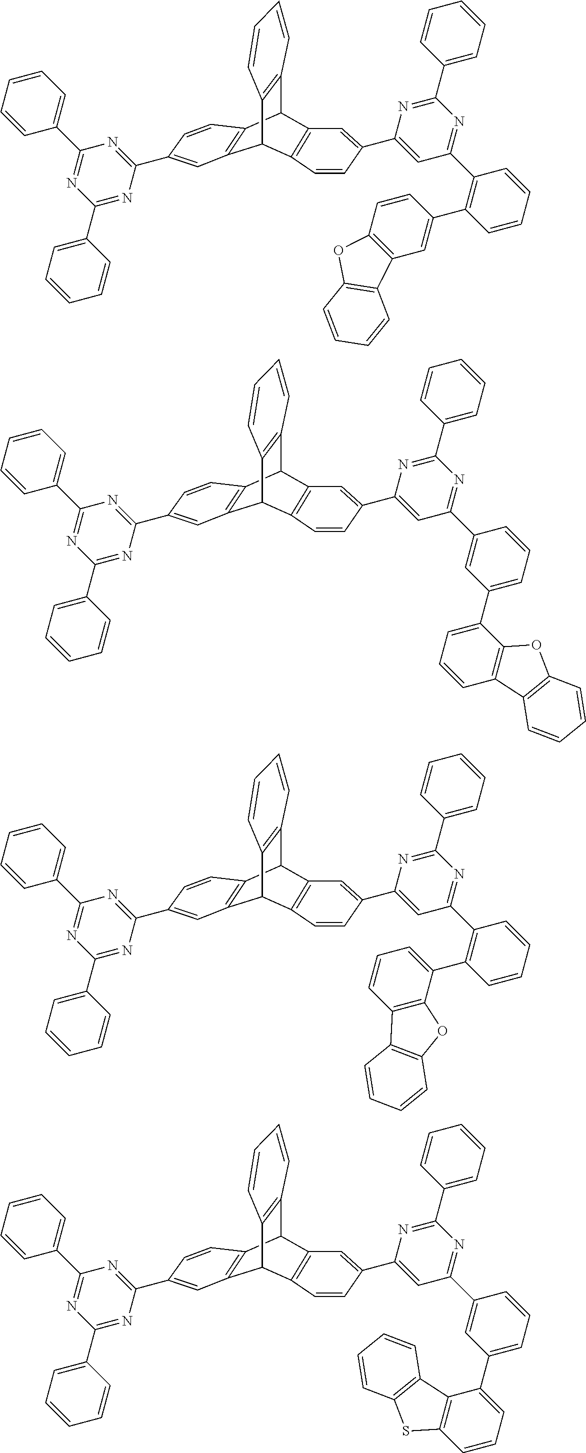

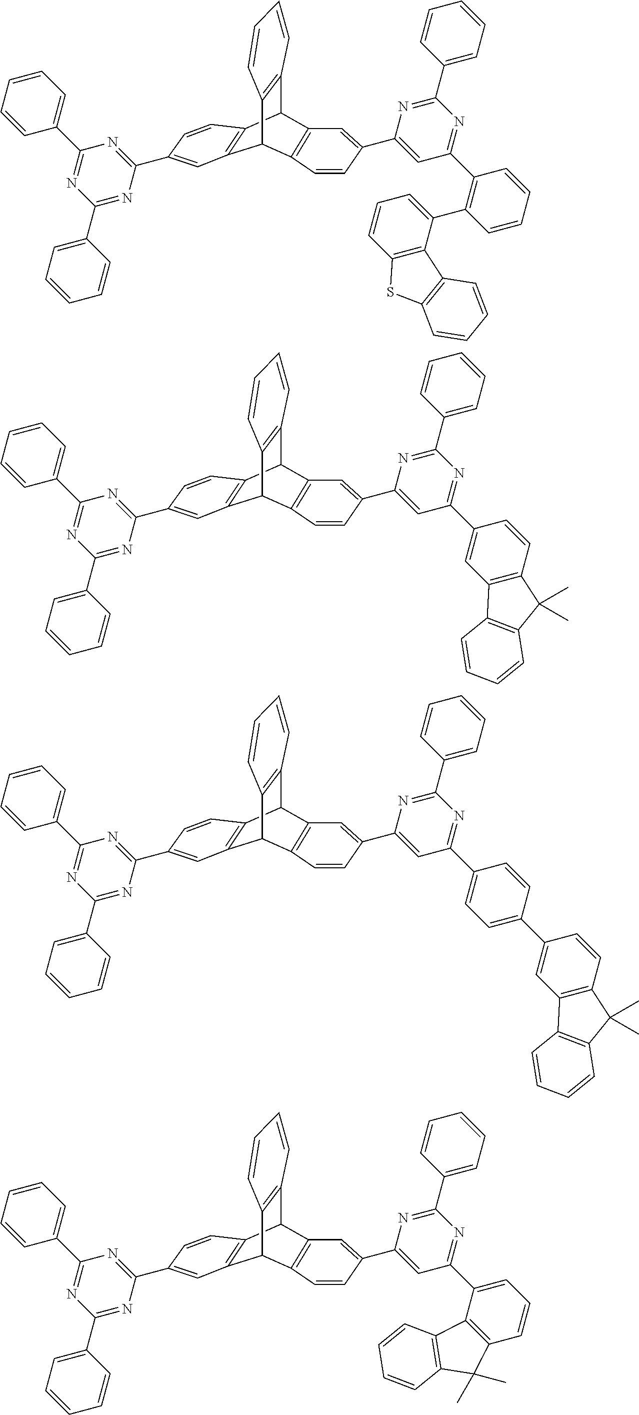

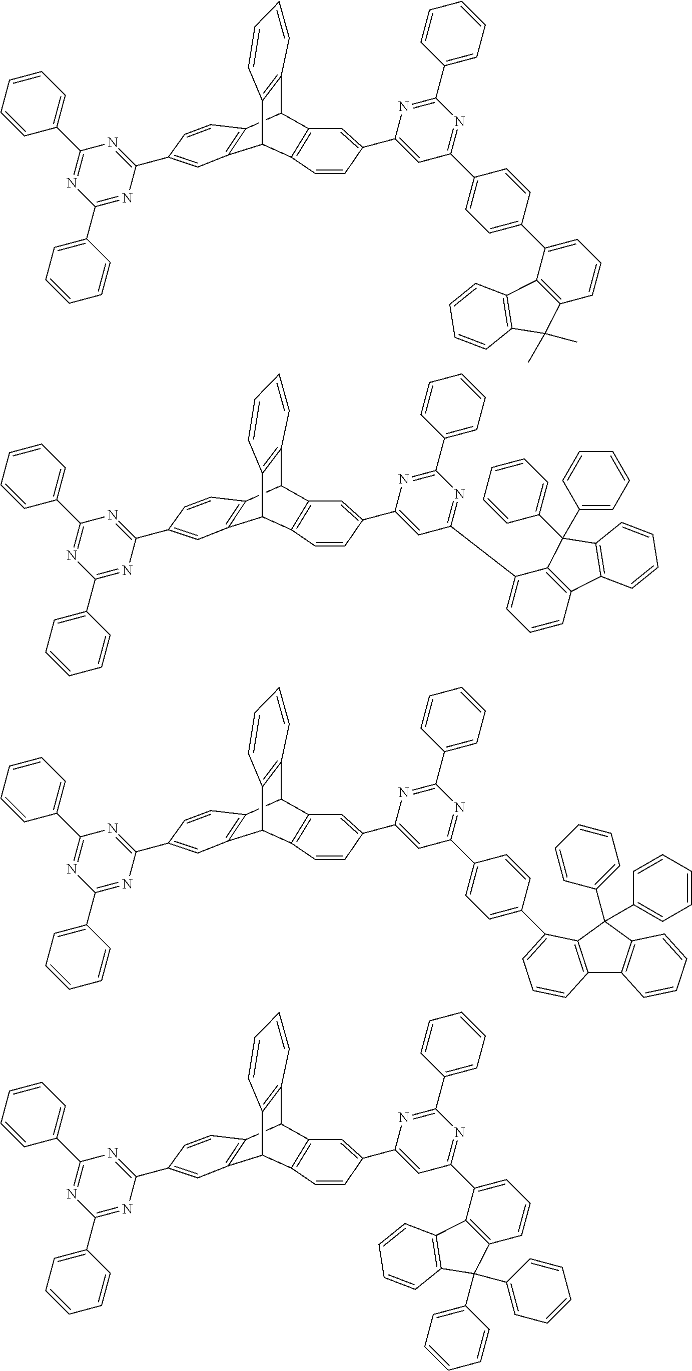

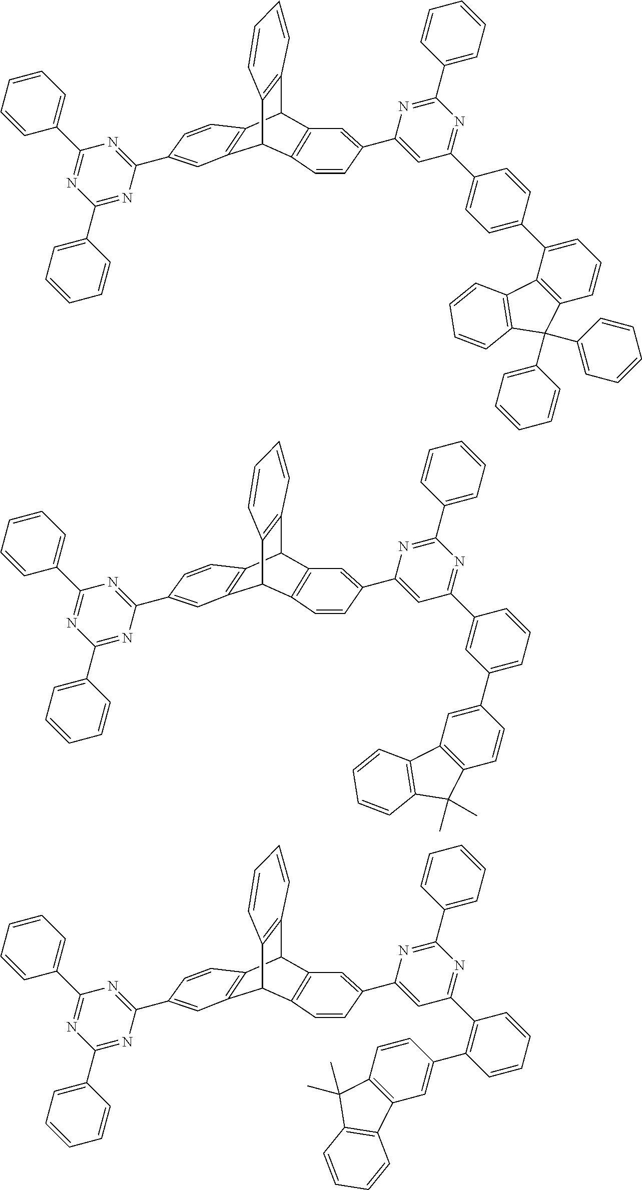

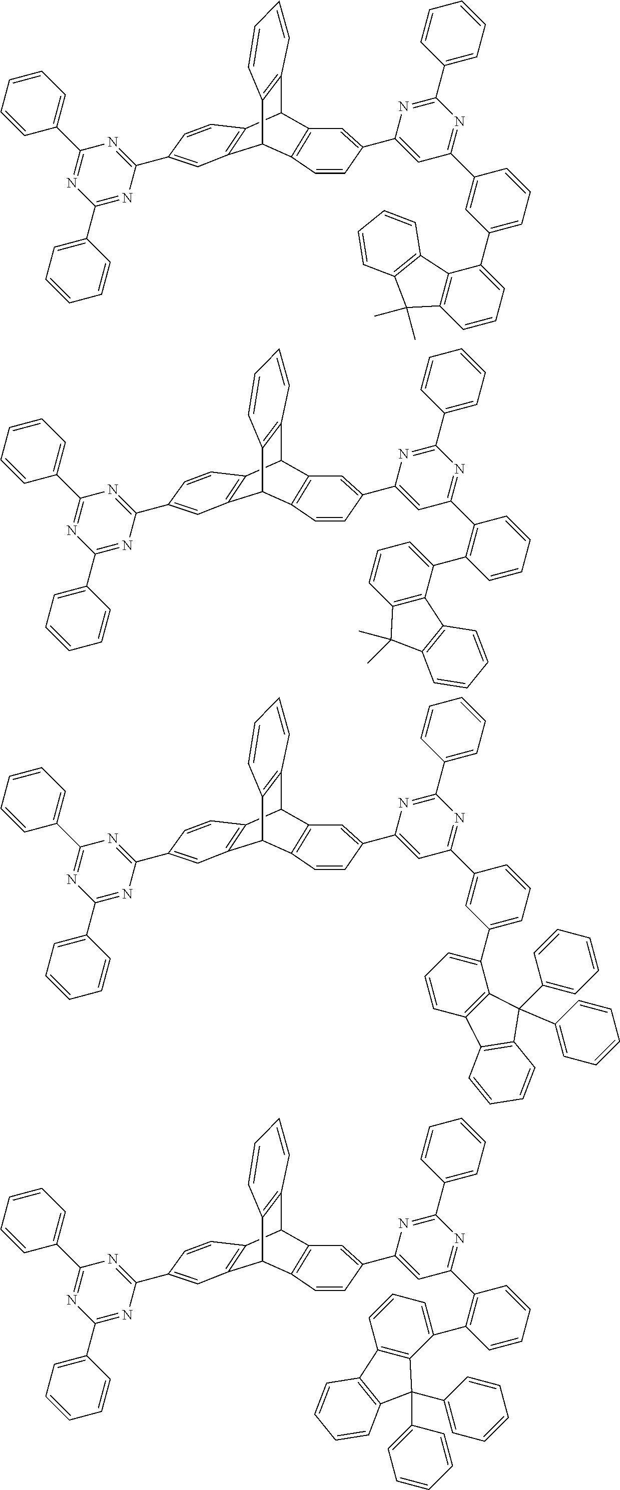

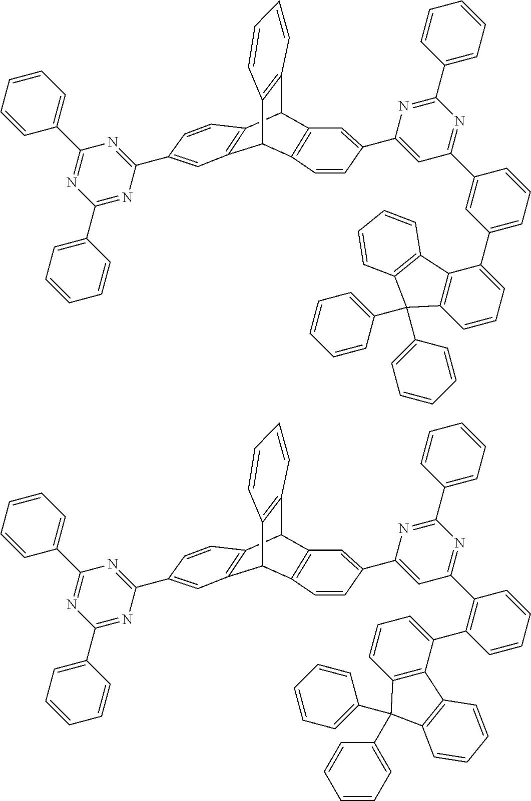

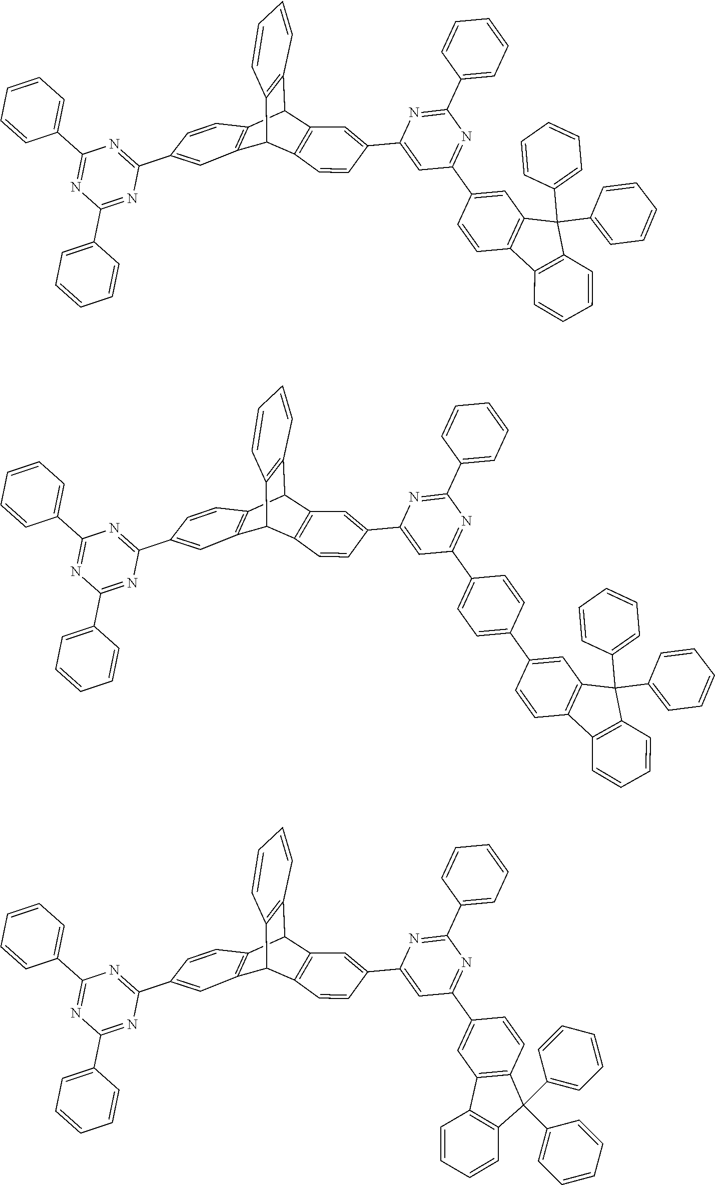 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)C(c3ccccc3)(c3ccccc3)c3ccccc3-4)n2)cc1 ; c1ccc(-c2nc(-c3ccc(-c4ccc5c(c4)C(c4ccccc4)(c4ccccc4)c4ccccc4-5)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)-c3ccccc3C4(c3ccccc3)c3ccccc3)cc(-c3ccc4c(c3)C3c5ccccc5C4c4cc(-c5nc(-c6ccccc6)nc(-c6ccccc6)n5)ccc43)n2)cc1]

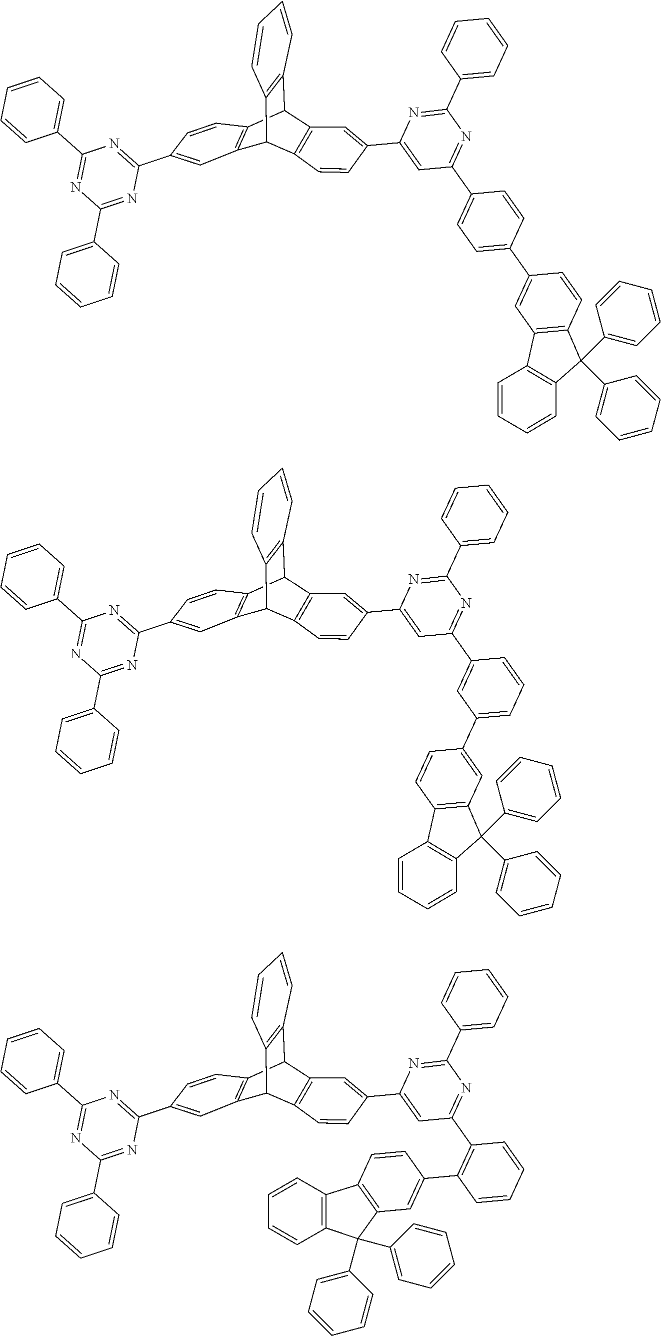 [SMILES: c1ccc(-c2nc(-c3ccc(-c4ccc5c(c4)-c4ccccc4C5(c4ccccc4)c4ccccc4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3cccc(-c4ccc5c(c4)C(c4ccccc4)(c4ccccc4)c4ccccc4-5)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)C(c3ccccc3)(c3ccccc3)c3ccccc3-4)n2)cc1]

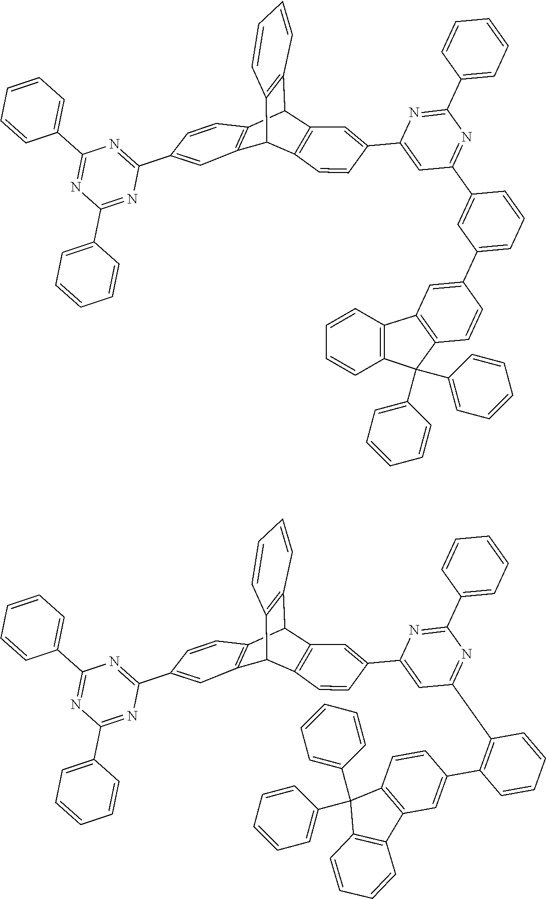 [SMILES: c1ccc(-c2nc(-c3cccc(-c4ccc5c(c4)-c4ccccc4C5(c4ccccc4)c4ccccc4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(Cc3ccccc3-c3ccc4c(c3)-c3ccccc3C4(c3ccccc3)c3ccccc3)cc(-c3ccc4c(c3)C3c5ccccc5C4c4cc(-c5nc(-c6ccccc6)nc(-c6ccccc6)n5)ccc43)n2)cc1]

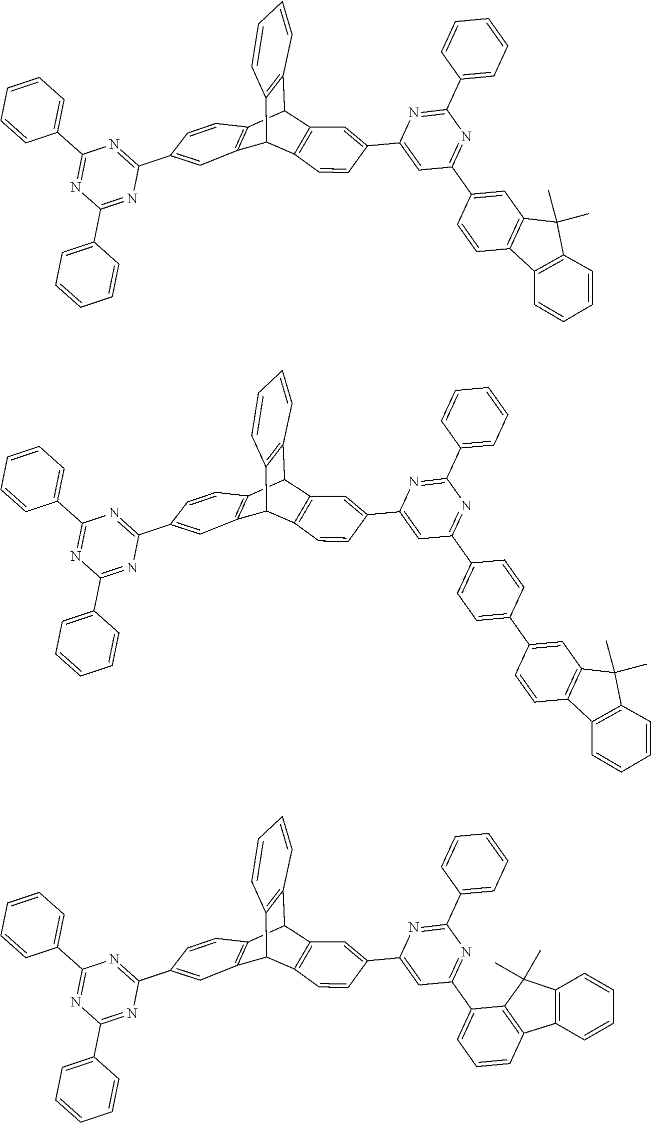 [SMILES: CC1(C)c2ccccc2-c2ccc(-c3cc(-c4ccc5c(c4)C4c6ccccc6C5c5cc(-c6nc(-c7ccccc7)nc(-c7ccccc7)n6)ccc54)nc(-c4ccccc4)n3)cc21 ; CC1(C)c2ccccc2-c2ccc(-c3ccc(-c4cc(-c5ccc6c(c5)C5c7ccccc7C6c6cc(-c7nc(-c8ccccc8)nc(-c8ccccc8)n7)ccc65)nc(-c5ccccc5)n4)cc3)cc21 ; CC1(C)c2ccccc2-c2cccc(-c3cc(-c4ccc5c(c4)C4c6ccccc6C5c5cc(-c6nc(-c7ccccc7)nc(-c7ccccc7)n6)ccc54)nc(-c4ccccc4)n3)c21]

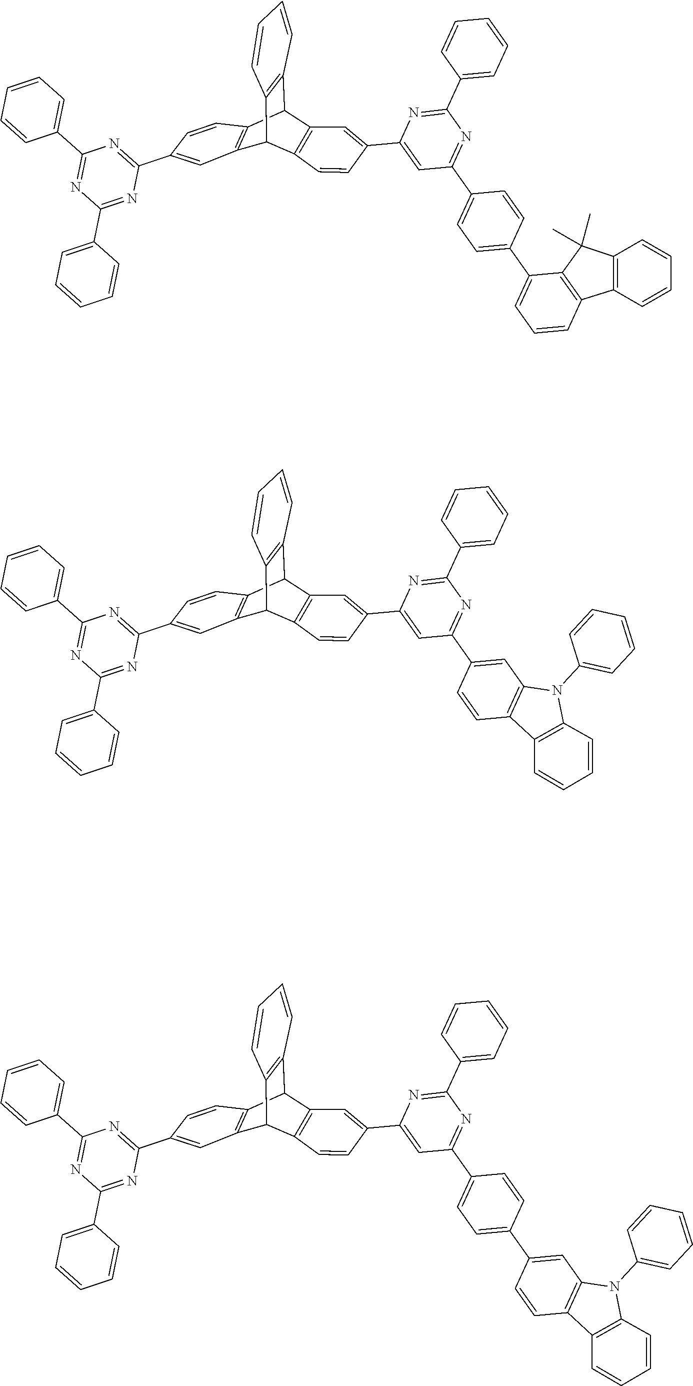 [SMILES: CC1(C)c2ccccc2-c2cccc(-c3ccc(-c4cc(-c5ccc6c(c5)C5c7ccccc7C6c6cc(-c7nc(-c8ccccc8)nc(-c8ccccc8)n7)ccc65)nc(-c5ccccc5)n4)cc3)c21 ; c1ccc(-c2nc(-c3ccc4c(c3)C3c5ccccc5C4c4cc(-c5nc(-c6ccccc6)nc(-c6ccccc6)n5)ccc43)cc(-c3ccc4c5ccccc5n(-c5ccccc5)c4c3)n2)cc1 ; c1ccc(-c2nc(-c3ccc(-c4ccc5c6ccccc6n(-c6ccccc6)c5c4)cc3)cc(-c3ccc4c(c3)C3c5ccccc5C4c4cc(-c5nc(-c6ccccc6)nc(-c6ccccc6)n5)ccc43)n2)cc1]

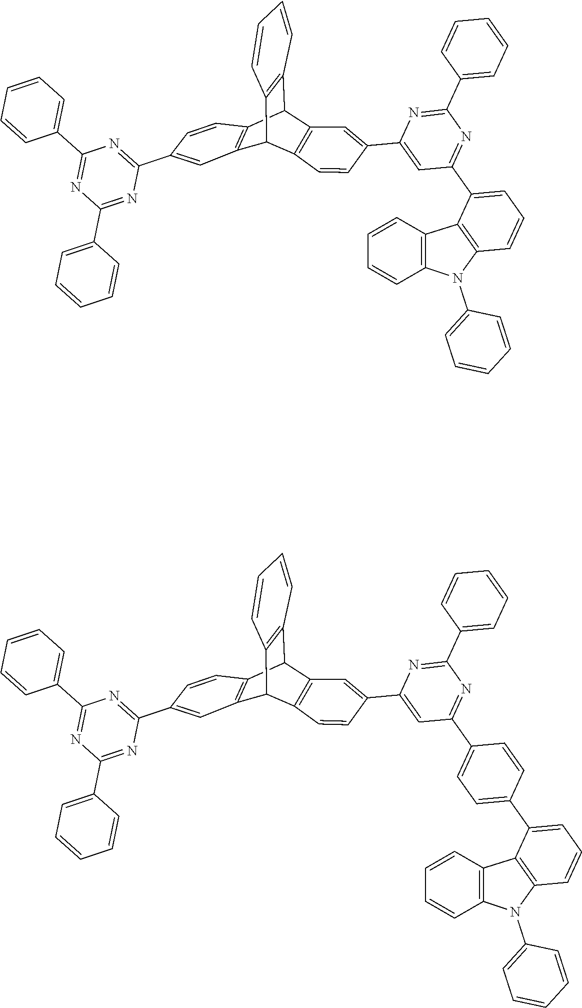 [SMILES: c1ccc(-c2nc(-c3ccc4c(c3)C3c5ccccc5C4c4cc(-c5nc(-c6ccccc6)nc(-c6ccccc6)n5)ccc43)cc(-c3cccc4c3c3ccccc3n4-c3ccccc3)n2)cc1 ; c1ccc(-c2nc(-c3ccc(-c4cccc5c4c4ccccc4n5-c4ccccc4)cc3)cc(-c3ccc4c(c3)C3c5ccccc5C4c4cc(-c5nc(-c6ccccc6)nc(-c6ccccc6)n5)ccc43)n2)cc1]

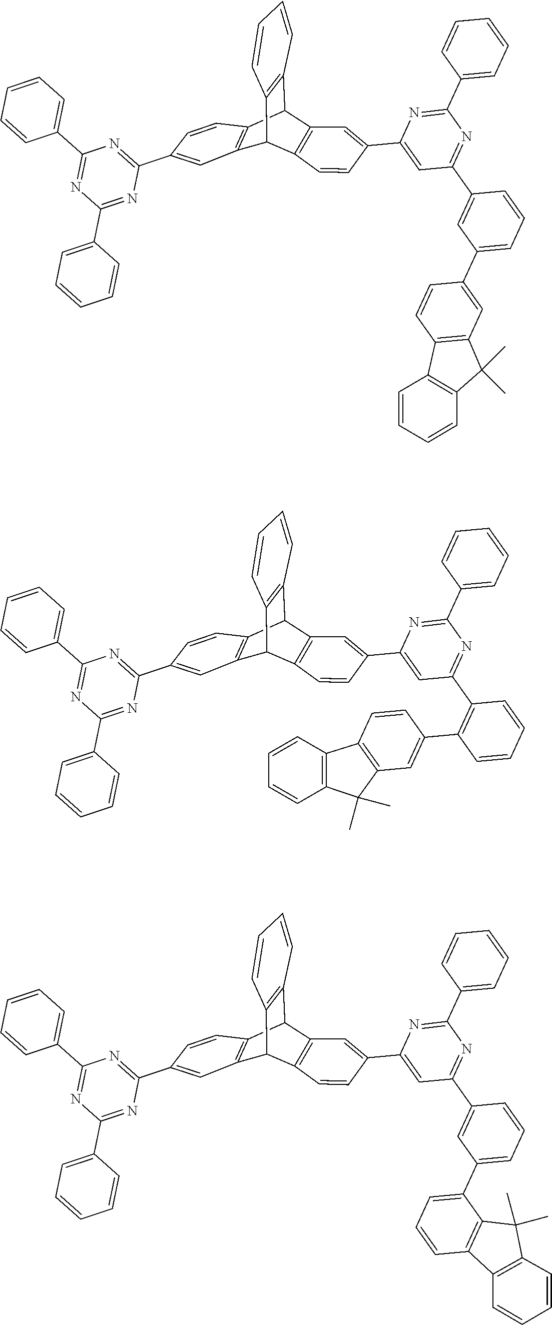 [SMILES: CC1(C)c2ccccc2-c2ccc(-c3cccc(-c4cc(-c5ccc6c(c5)C5c7ccccc7C6c6cc(-c7nc(-c8ccccc8)nc(-c8ccccc8)n7)ccc65)nc(-c5ccccc5)n4)c3)cc21 ; CC1(C)c2ccccc2-c2ccc(-c3ccccc3-c3cc(-c4ccc5c(c4)C4c6ccccc6C5c5cc(-c6nc(-c7ccccc7)nc(-c7ccccc7)n6)ccc54)nc(-c4ccccc4)n3)cc21 ; CC1(C)c2ccccc2-c2cccc(-c3cccc(-c4cc(-c5ccc6c(c5)C5c7ccccc7C6c6cc(-c7nc(-c8ccccc8)nc(-c8ccccc8)n7)ccc65)nc(-c5ccccc5)n4)c3)c21]

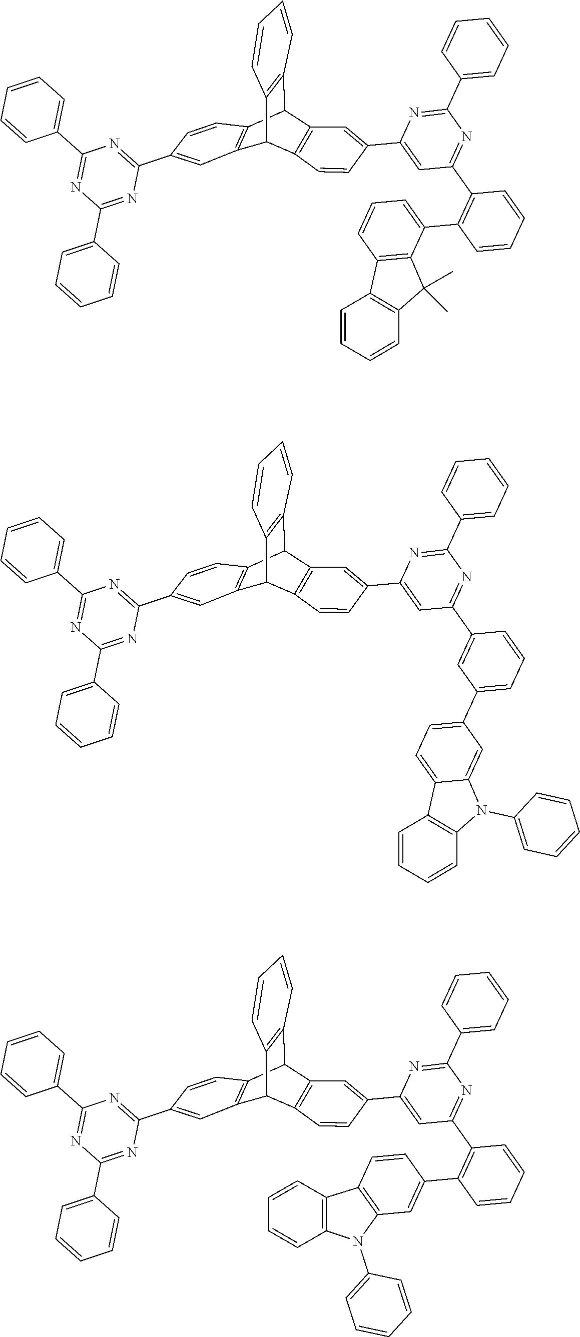 [SMILES: CC1(C)c2ccccc2-c2cccc(-c3ccccc3-c3cc(-c4ccc5c(c4)C4c6ccccc6C5c5cc(-c6nc(-c7ccccc7)nc(-c7ccccc7)n6)ccc54)nc(-c4ccccc4)n3)c21 ; c1ccc(-c2nc(-c3cccc(-c4ccc5c6ccccc6n(-c6ccccc6)c5c4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c5ccccc5n(-c5ccccc5)c4c3)n2)cc1]

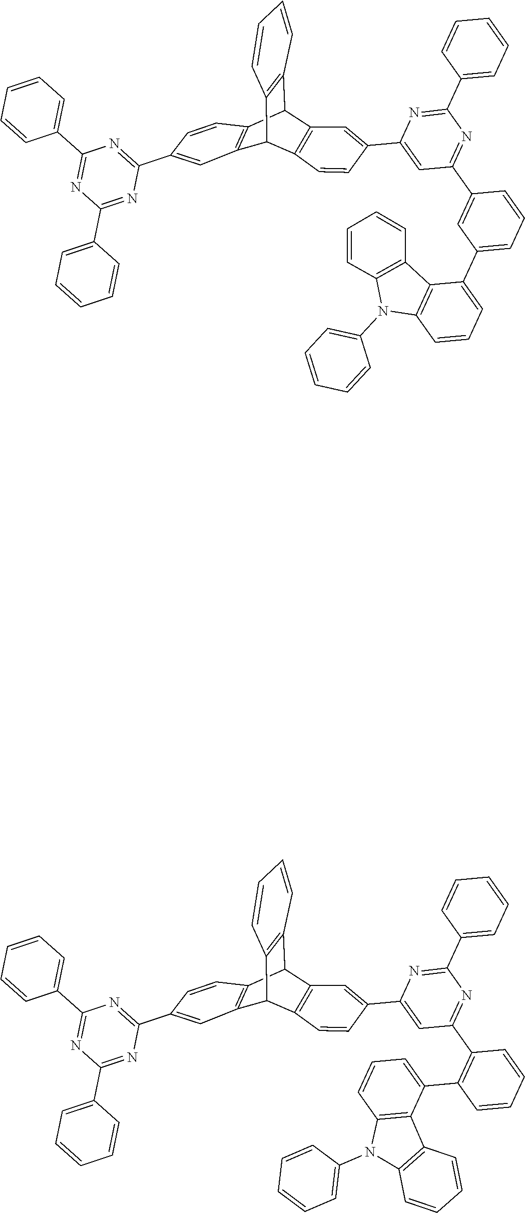 [SMILES: c1ccc(-c2nc(-c3cccc(-c4cccc5c4c4ccccc4n5-c4ccccc4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccc4c3c3ccccc3n4-c3ccccc3)n2)cc1]

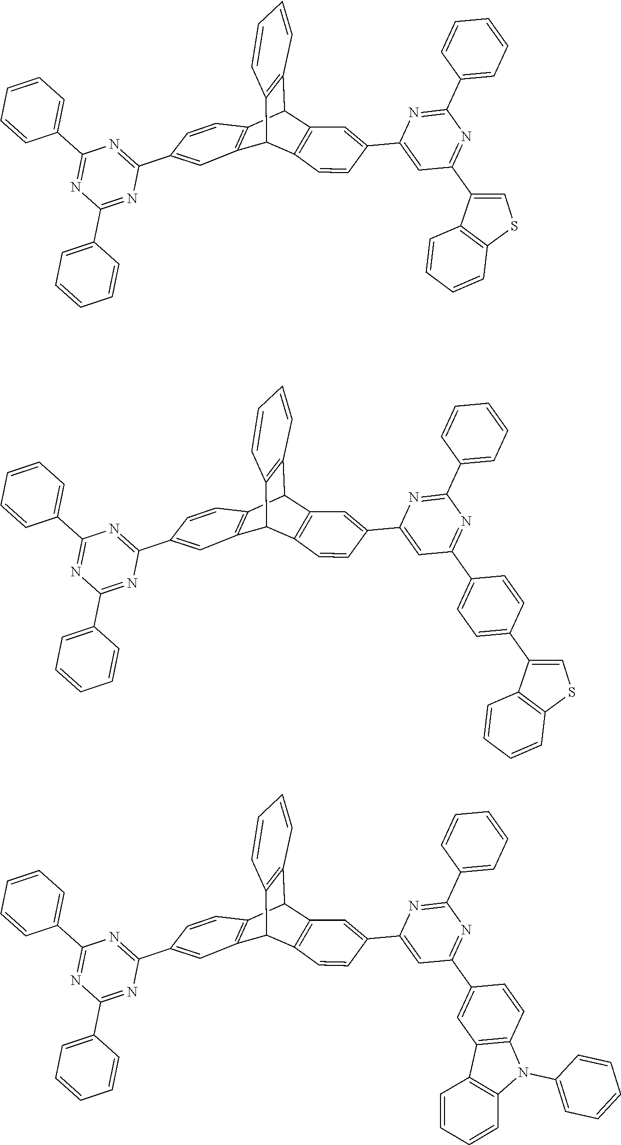 [SMILES: c1ccc(-c2nc(-c3ccc4c(c3)C3c5ccccc5C4c4cc(-c5nc(-c6ccccc6)nc(-c6ccccc6)n5)ccc43)cc(-c3csc4ccccc34)n2)cc1 ; c1ccc(-c2nc(-c3ccc(-c4csc5ccccc45)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)c3ccccc3n4-c3ccccc3)n2)cc1]

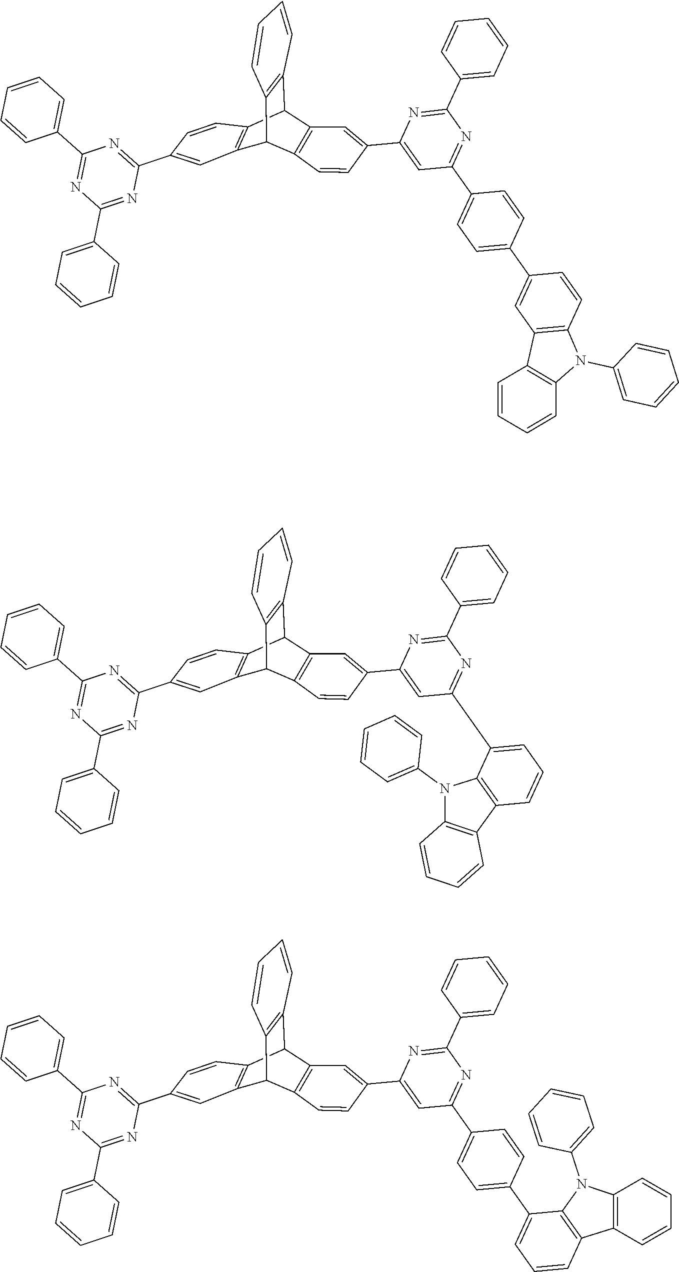 [SMILES: c1ccc(-c2nc(-c3ccc(-c4ccc5c(c4)c4ccccc4n5-c4ccccc4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cccc4c5ccccc5n(-c5ccccc5)c34)n2)cc1 ; c1ccc(-c2nc(-c3ccc(-c4cccc5c6ccccc6n(-c6ccccc6)c45)cc3)cc(-c3ccc4c(c3)C3c5ccccc5C4c4cc(-c5nc(-c6ccccc6)nc(-c6ccccc6)n5)ccc43)n2)cc1]

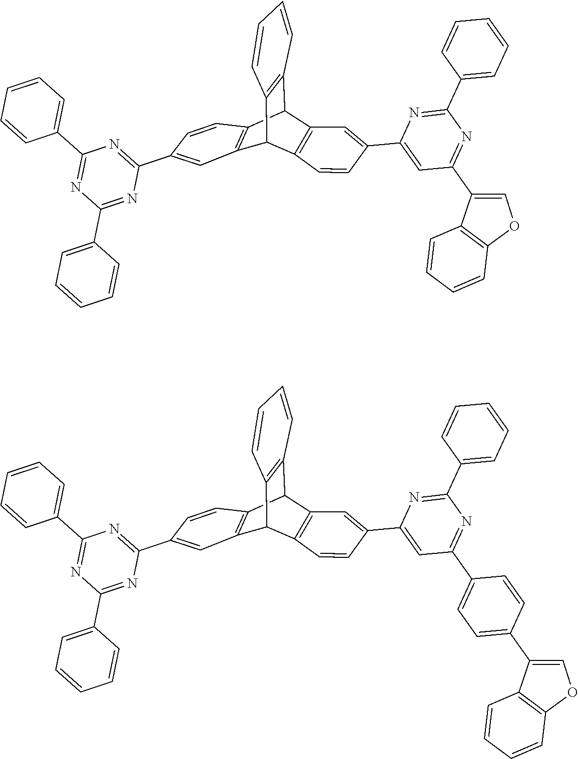 [SMILES: c1ccc(-c2nc(-c3ccc4c(c3)C3c5ccccc5C4c4cc(-c5nc(-c6ccccc6)nc(-c6ccccc6)n5)ccc43)cc(-c3coc4ccccc34)n2)cc1 ; c1ccc(-c2nc(-c3ccc(-c4coc5ccccc45)cc3)cc(-c3ccc4c(c3)C3c5ccccc5C4c4cc(-c5nc(-c6ccccc6)nc(-c6ccccc6)n5)ccc43)n2)cc1]

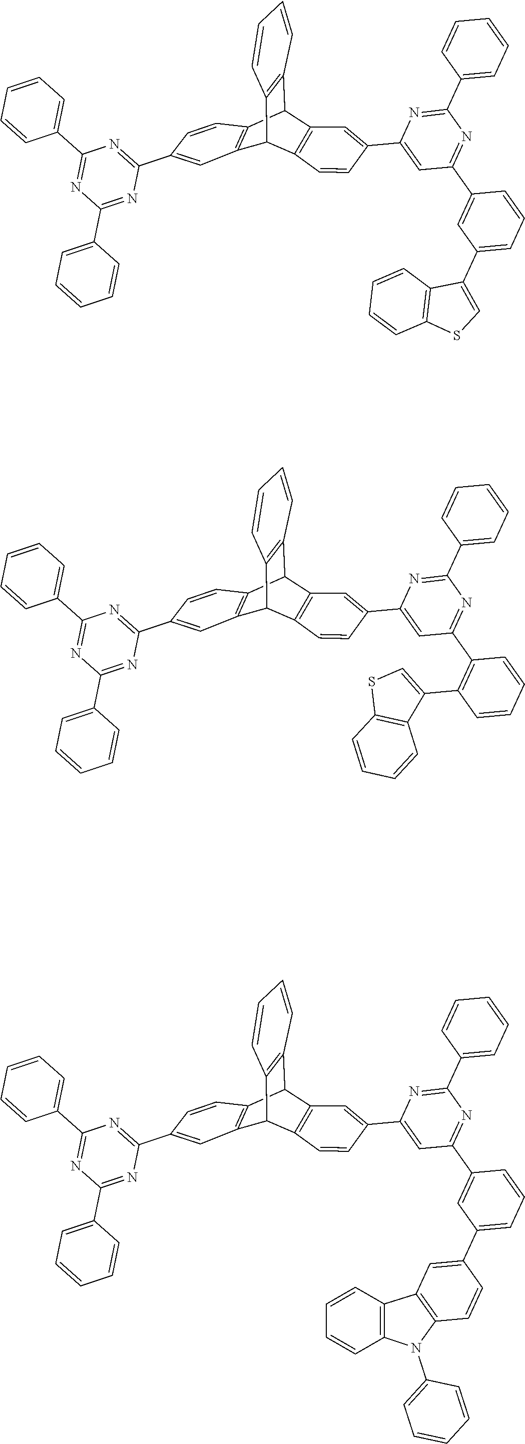 [SMILES: c1ccc(-c2nc(-c3cccc(-c4csc5ccccc45)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3csc4ccccc34)n2)cc1 ; c1ccc(-c2nc(-c3cccc(-c4ccc5c(c4)c4ccccc4n5-c4ccccc4)c3)cc(-c3ccc4c(c3)C3c5ccccc5C4c4cc(-c5nc(-c6ccccc6)nc(-c6ccccc6)n5)ccc43)n2)cc1]

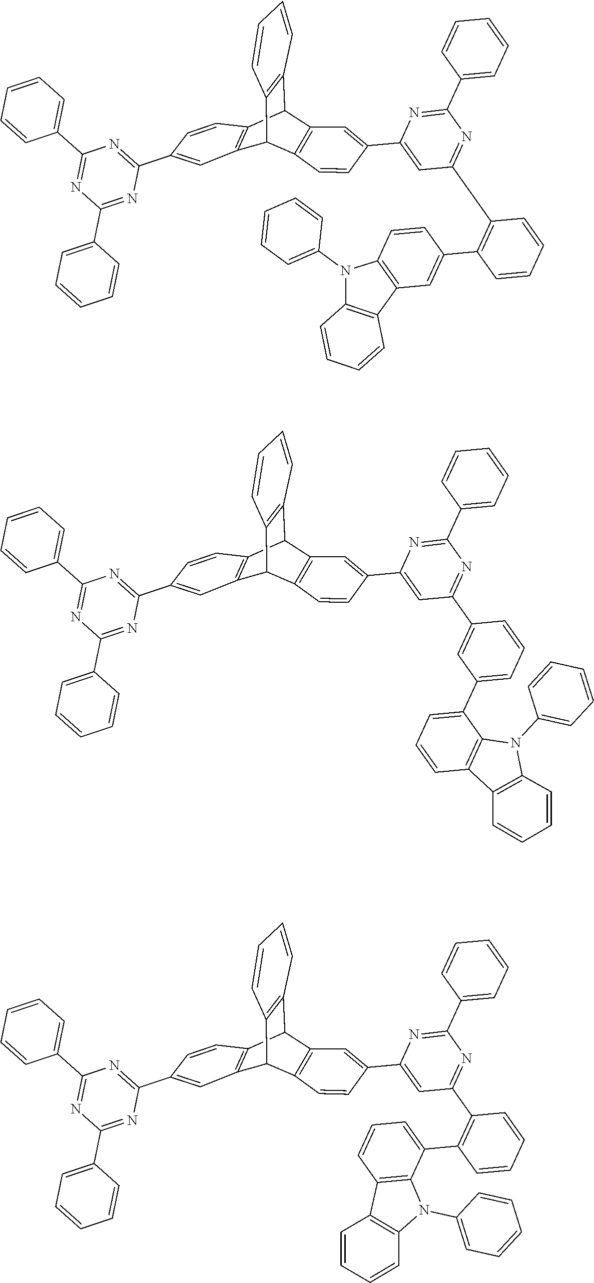 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)c3ccccc3n4-c3ccccc3)n2)cc1 ; c1ccc(-c2nc(-c3cccc(-c4cccc5c6ccccc6n(-c6ccccc6)c45)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccc4c5ccccc5n(-c5ccccc5)c34)n2)cc1]

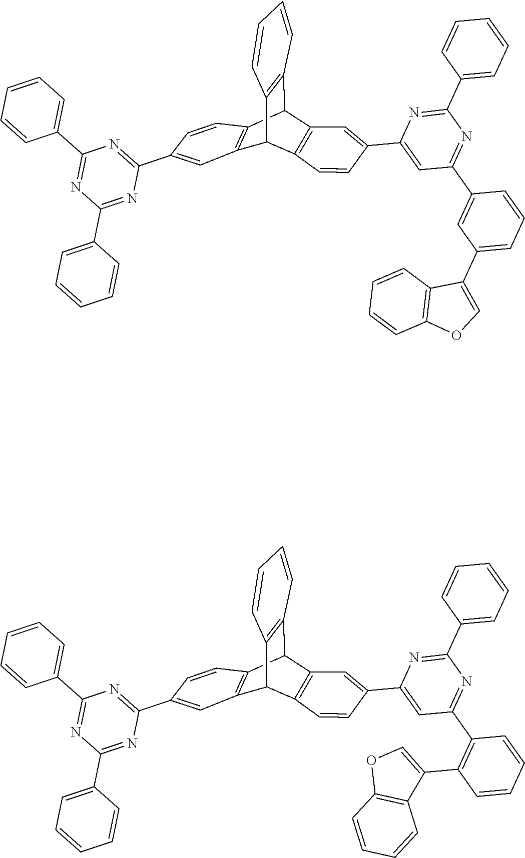 [SMILES: c1ccc(-c2nc(-c3cccc(-c4coc5ccccc45)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3coc4ccccc34)n2)cc1]

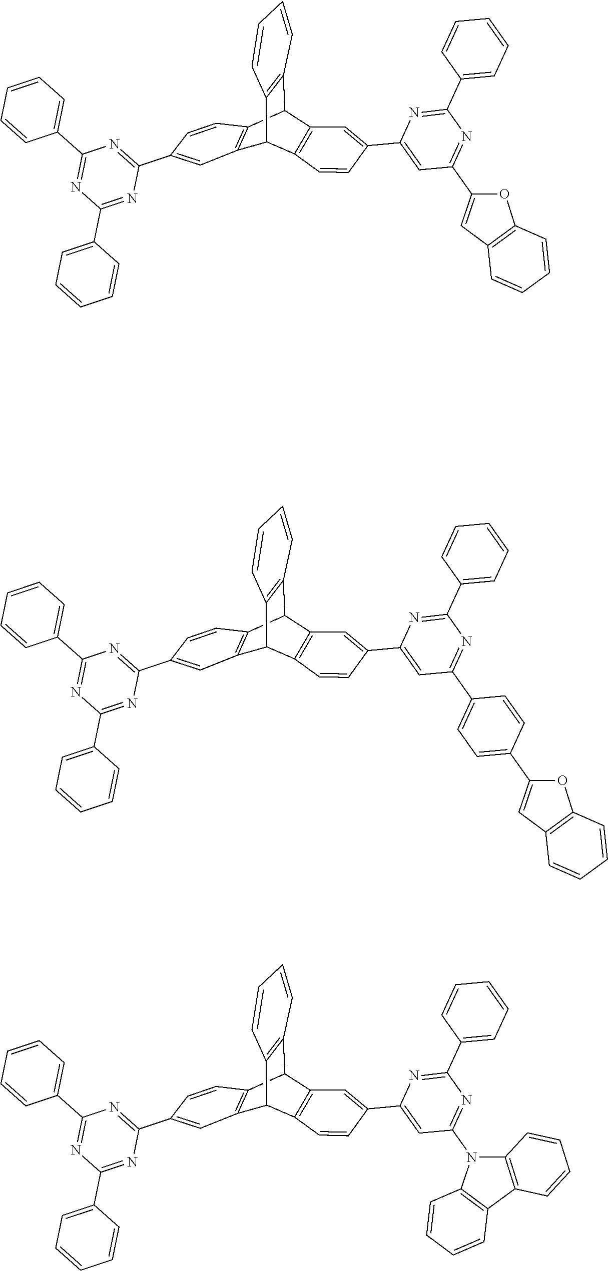 [SMILES: c1ccc(-c2nc(-c3ccc4c(c3)C3c5ccccc5C4c4cc(-c5nc(-c6ccccc6)nc(-c6ccccc6)n5)ccc43)cc(-c3cc4ccccc4o3)n2)cc1 ; c1ccc(-c2nc(-c3ccc(-c4cc5ccccc5o4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-n3c4ccccc4c4ccccc43)n2)cc1]

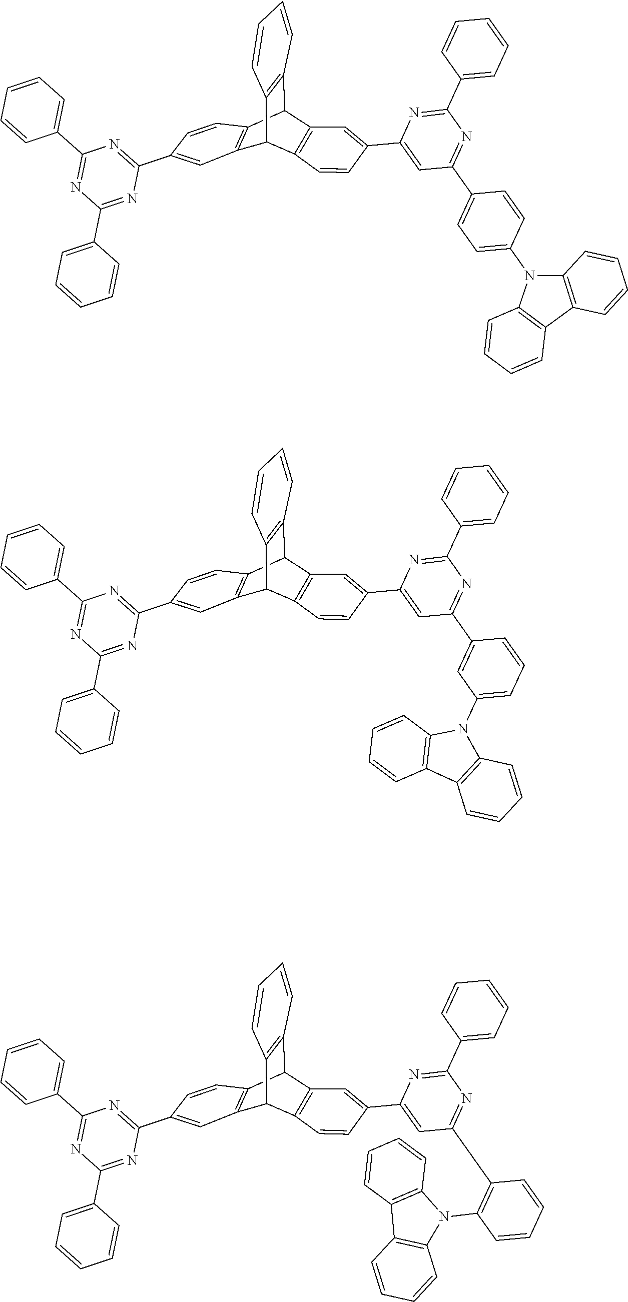 [SMILES: c1ccc(-c2nc(-c3ccc(-n4c5ccccc5c5ccccc54)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3cccc(-n4c5ccccc5c5ccccc54)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-n3c4ccccc4c4ccccc43)n2)cc1]

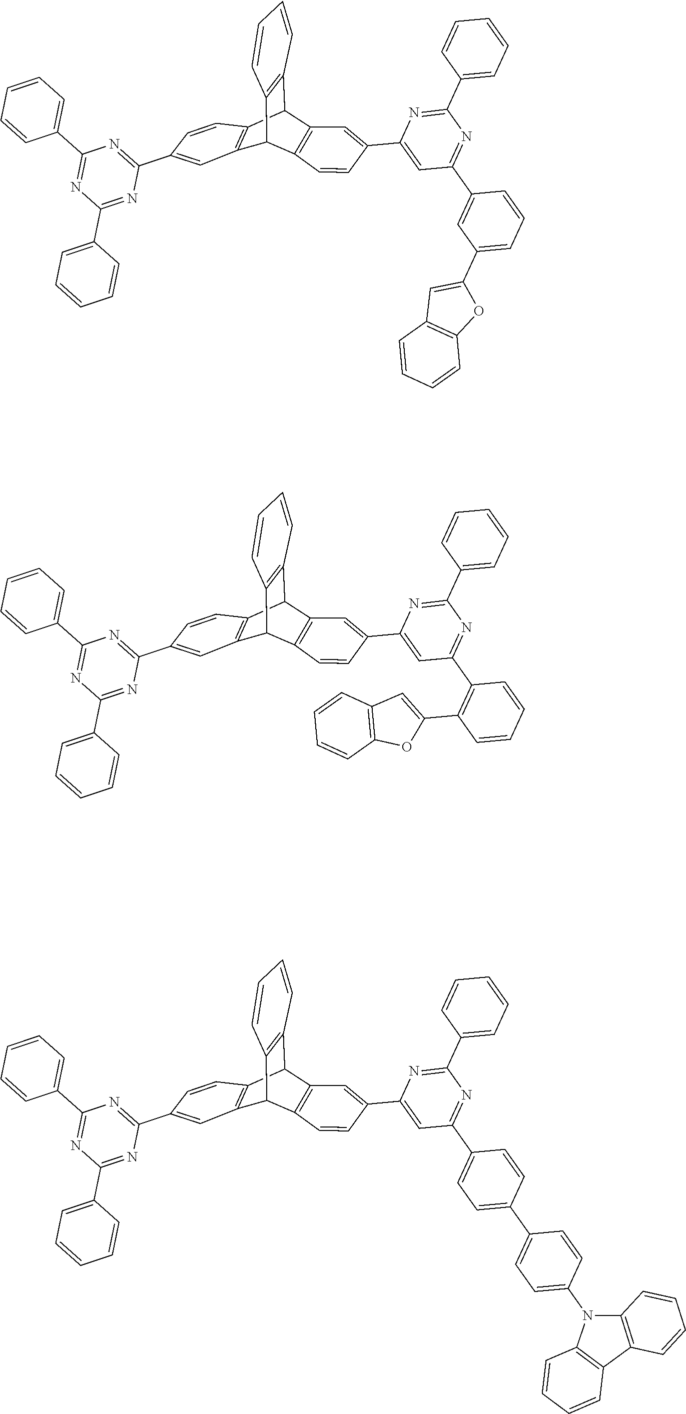 [SMILES: c1ccc(-c2nc(-c3cccc(-c4cc5ccccc5o4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cc4ccccc4o3)n2)cc1 ; c1ccc(-c2nc(-c3ccc(-c4ccc(-n5c6ccccc6c6ccccc65)cc4)cc3)cc(-c3ccc4c(c3)C3c5ccccc5C4c4cc(-c5nc(-c6ccccc6)nc(-c6ccccc6)n5)ccc43)n2)cc1]

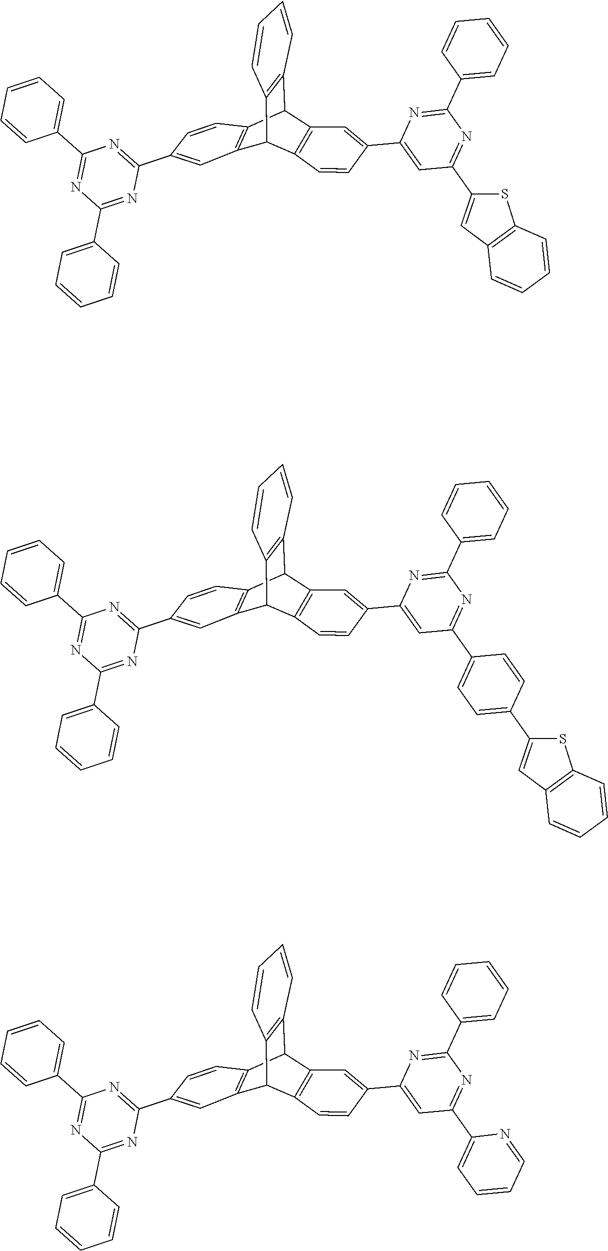 [SMILES: c1ccc(-c2nc(-c3ccc4c(c3)C3c5ccccc5C4c4cc(-c5nc(-c6ccccc6)nc(-c6ccccc6)n5)ccc43)cc(-c3cc4ccccc4s3)n2)cc1 ; c1ccc(-c2nc(-c3ccc(-c4cc5ccccc5s4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccn3)n2)cc1]

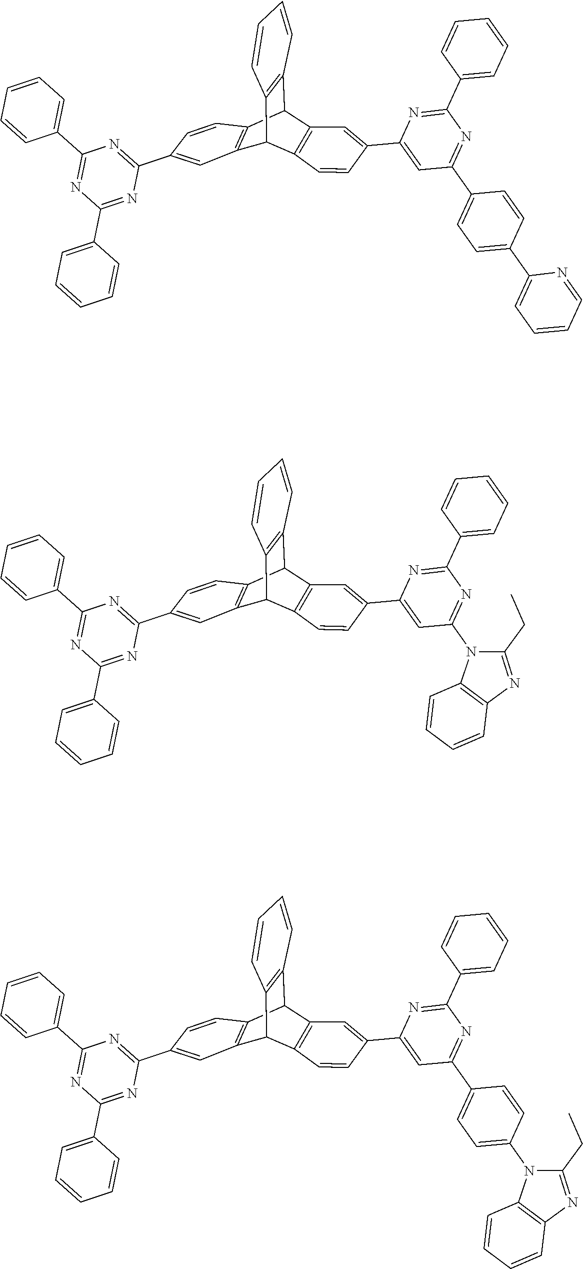 [SMILES: c1ccc(-c2nc(-c3ccc(-c4ccccn4)cc3)cc(-c3ccc4c(c3)C3c5ccccc5C4c4cc(-c5nc(-c6ccccc6)nc(-c6ccccc6)n5)ccc43)n2)cc1 ; CCc1nc2ccccc2n1-c1cc(-c2ccc3c(c2)C2c4ccccc4C3c3cc(-c4nc(-c5ccccc5)nc(-c5ccccc5)n4)ccc32)nc(-c2ccccc2)n1 ; CCc1nc2ccccc2n1-c1ccc(-c2cc(-c3ccc4c(c3)C3c5ccccc5C4c4cc(-c5nc(-c6ccccc6)nc(-c6ccccc6)n5)ccc43)nc(-c3ccccc3)n2)cc1]

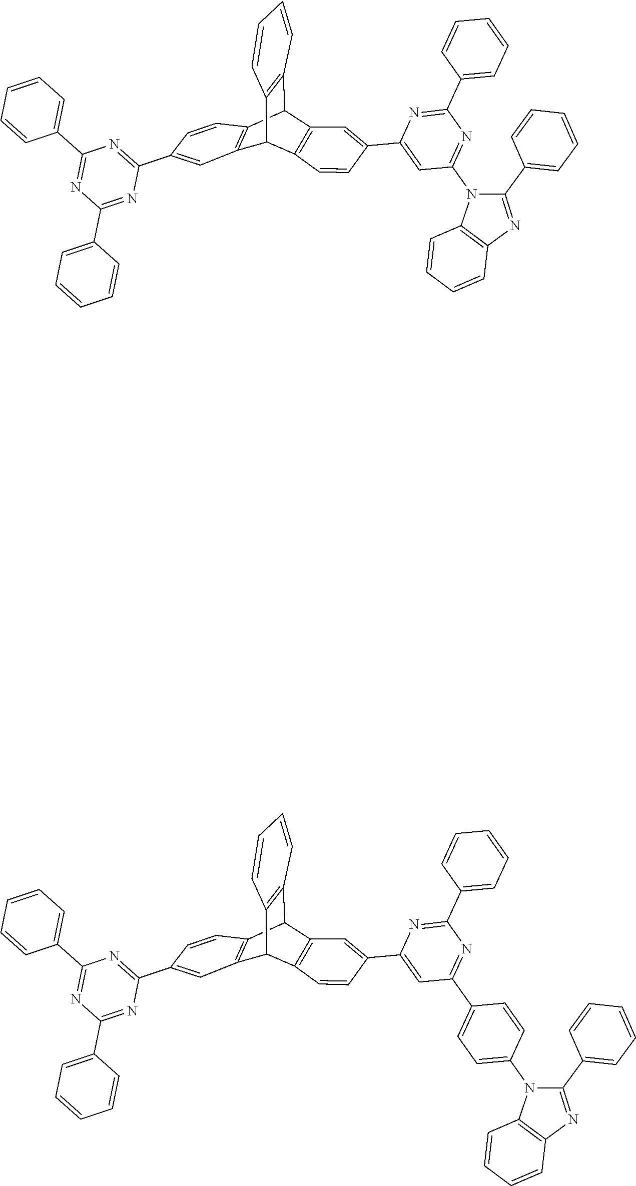 [SMILES: c1ccc(-c2nc(-c3ccc4c(c3)C3c5ccccc5C4c4cc(-c5nc(-c6ccccc6)nc(-c6ccccc6)n5)ccc43)cc(-n3c(-c4ccccc4)nc4ccccc43)n2)cc1 ; c1ccc(-c2nc(-c3ccc(-n4c(-c5ccccc5)nc5ccccc54)cc3)cc(-c3ccc4c(c3)C3c5ccccc5C4c4cc(-c5nc(-c6ccccc6)nc(-c6ccccc6)n5)ccc43)n2)cc1]

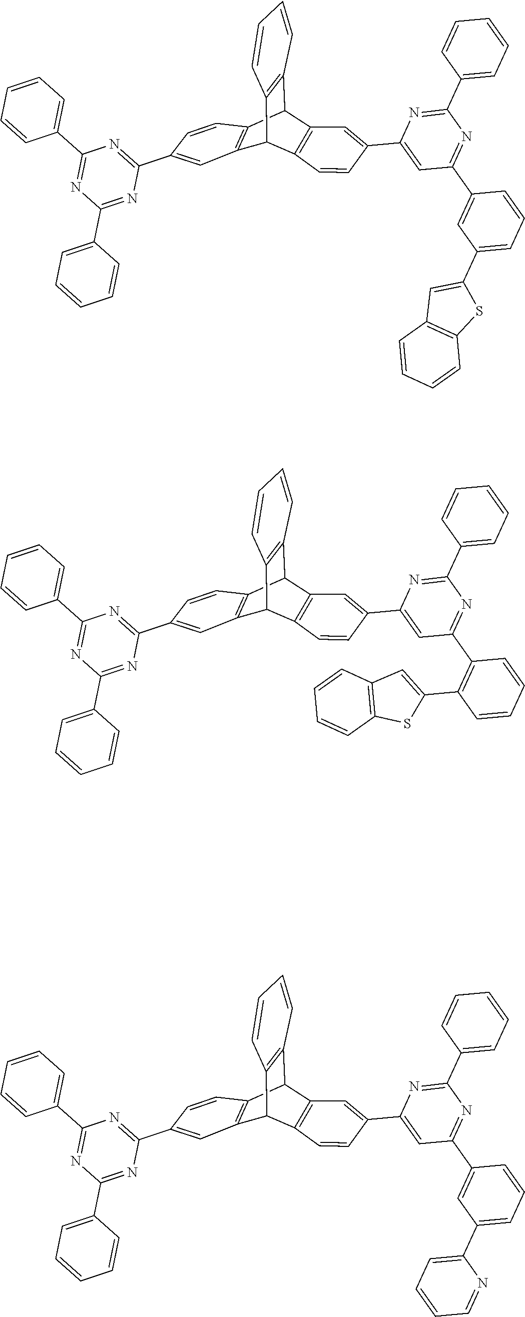 [SMILES: c1ccc(-c2nc(-c3cccc(-c4cc5ccccc5s4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cc4ccccc4s3)n2)cc1 ; c1ccc(-c2nc(-c3cccc(-c4ccccn4)c3)cc(-c3ccc4c(c3)C3c5ccccc5C4c4cc(-c5nc(-c6ccccc6)nc(-c6ccccc6)n5)ccc43)n2)cc1]

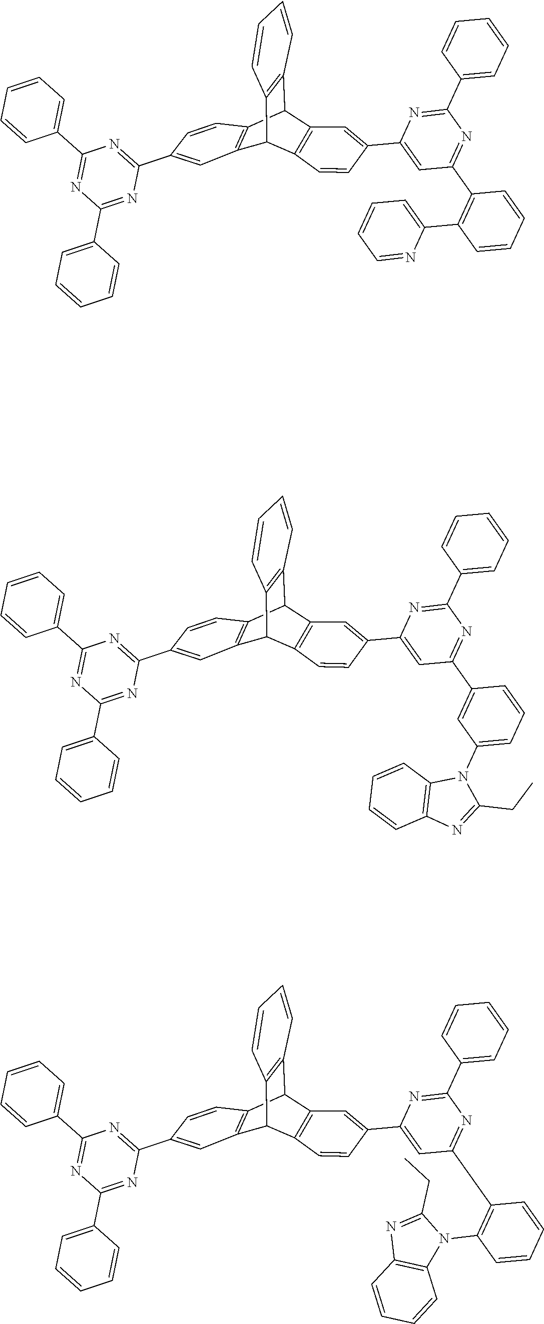 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccccn3)n2)cc1 ; CCc1nc2ccccc2n1-c1cccc(-c2cc(-c3ccc4c(c3)C3c5ccccc5C4c4cc(-c5nc(-c6ccccc6)nc(-c6ccccc6)n5)ccc43)nc(-c3ccccc3)n2)c1 ; CCc1nc2ccccc2n1-c1ccccc1-c1cc(-c2ccc3c(c2)C2c4ccccc4C3c3cc(-c4nc(-c5ccccc5)nc(-c5ccccc5)n4)ccc32)nc(-c2ccccc2)n1]

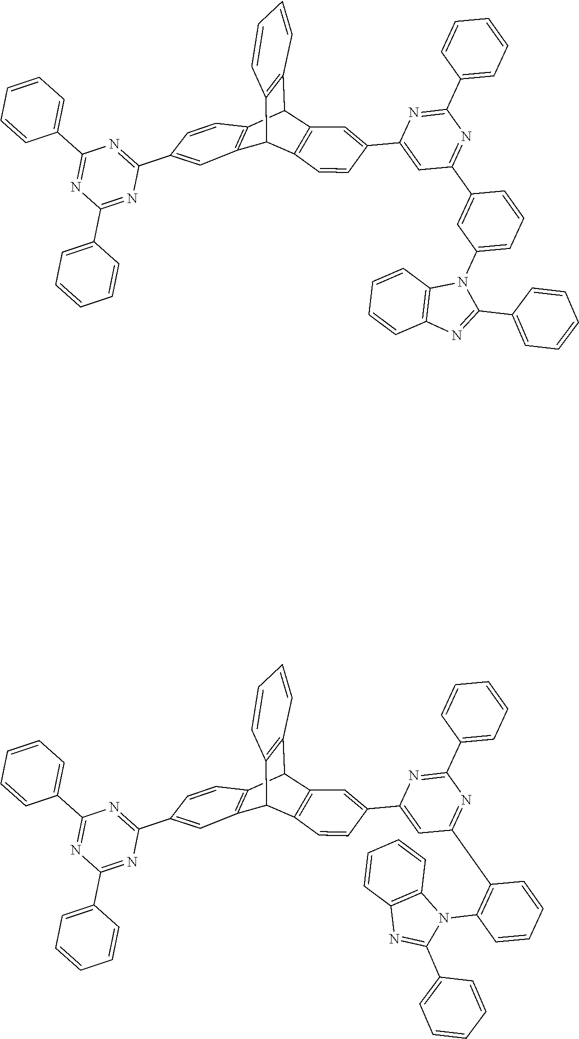 [SMILES: c1ccc(-c2nc(-c3cccc(-n4c(-c5ccccc5)nc5ccccc54)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-n3c(-c4ccccc4)nc4ccccc43)n2)cc1]

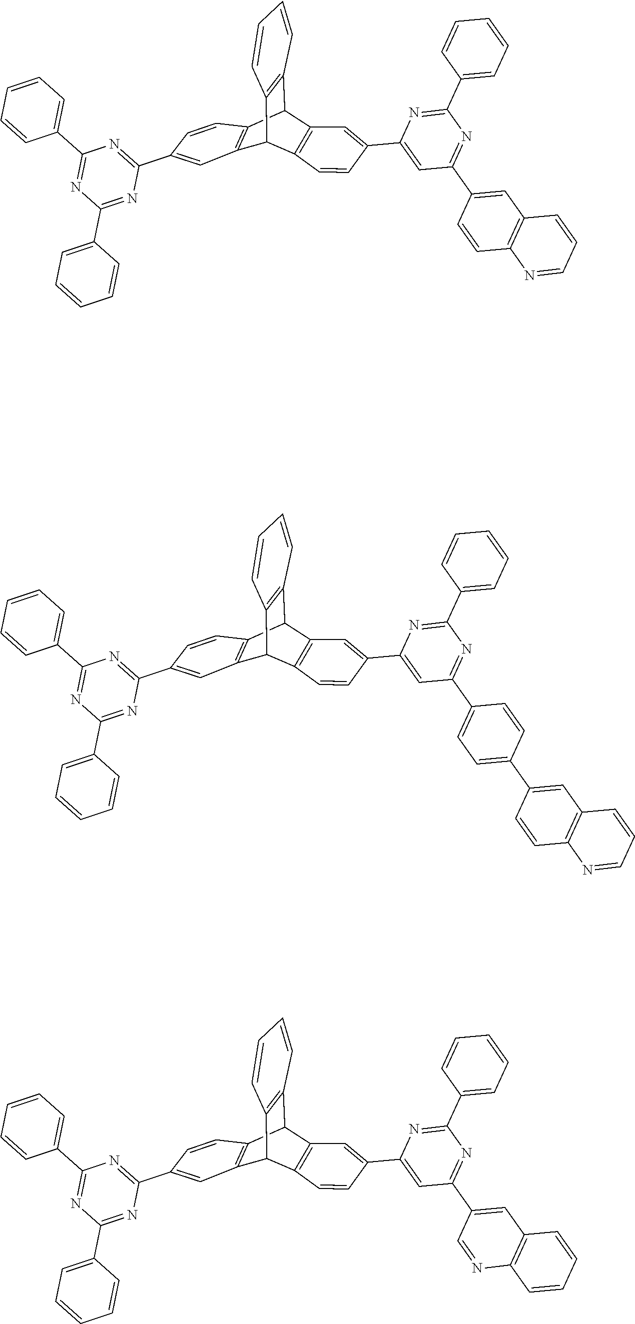 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccc4ncccc4c3)n2)cc1 ; c1ccc(-c2nc(-c3ccc(-c4ccc5ncccc5c4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cnc4ccccc4c3)n2)cc1]

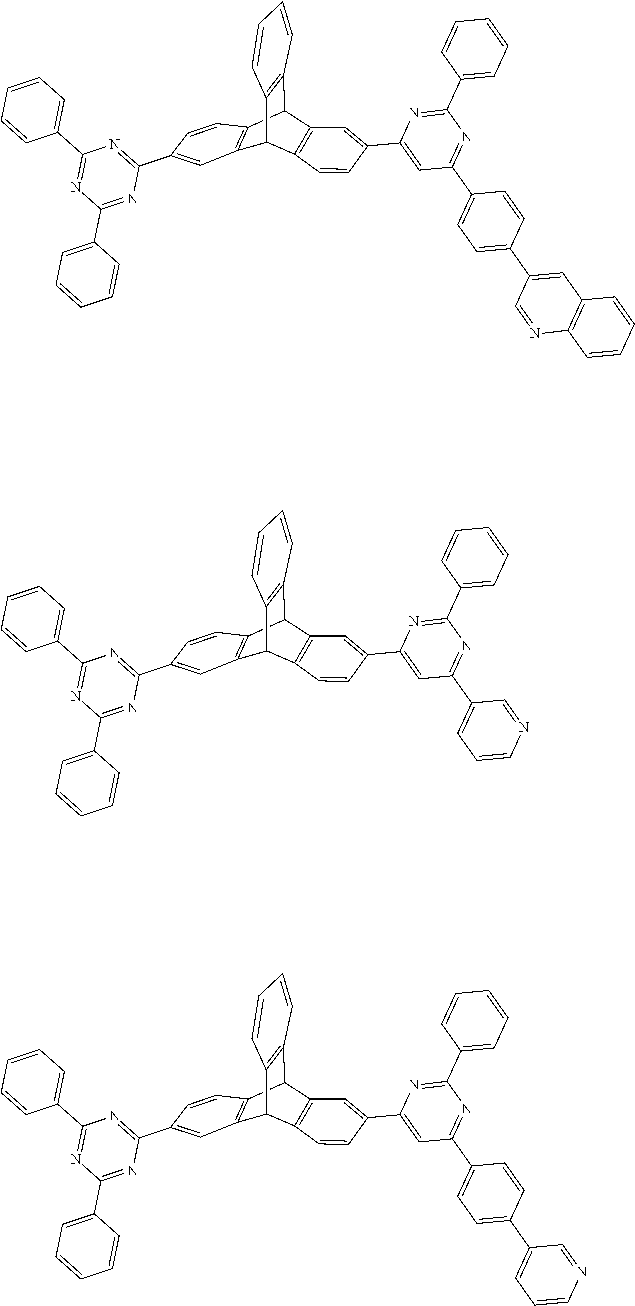 [SMILES: c1ccc(-c2nc(-c3ccc(-c4cnc5ccccc5c4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3cccnc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc(-c4cccnc4)cc3)cc(-c3ccc4c(c3)C3c5ccccc5C4c4cc(-c5nc(-c6ccccc6)nc(-c6ccccc6)n5)ccc43)n2)cc1]

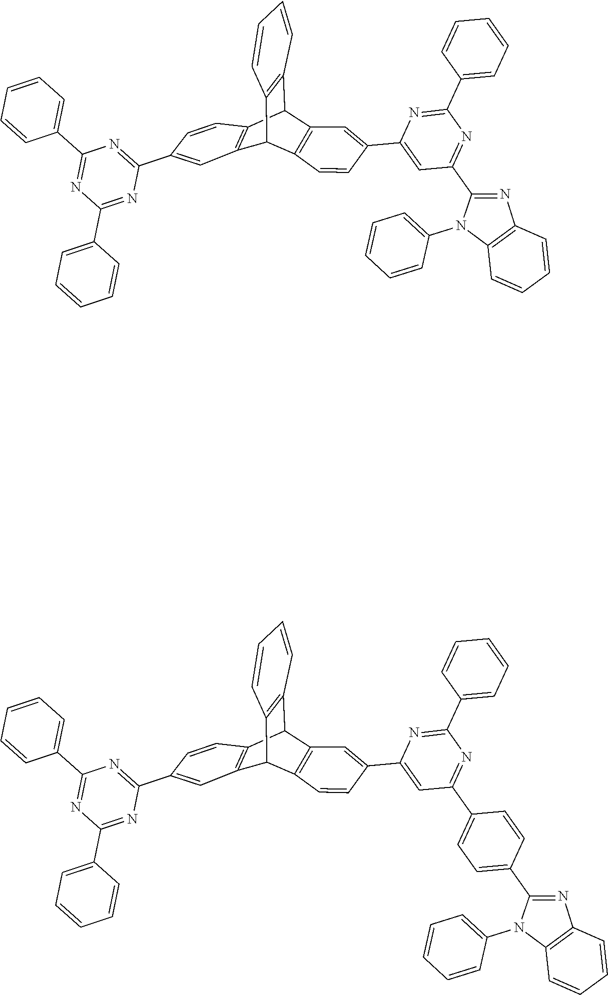 [SMILES: c1ccc(-c2nc(-c3ccc4c(c3)C3c5ccccc5C4c4cc(-c5nc(-c6ccccc6)nc(-c6ccccc6)n5)ccc43)cc(-c3nc4ccccc4n3-c3ccccc3)n2)cc1 ; c1ccc(-c2nc(-c3ccc(-c4nc5ccccc5n4-c4ccccc4)cc3)cc(-c3ccc4c(c3)C3c5ccccc5C4c4cc(-c5nc(-c6ccccc6)nc(-c6ccccc6)n5)ccc43)n2)cc1]

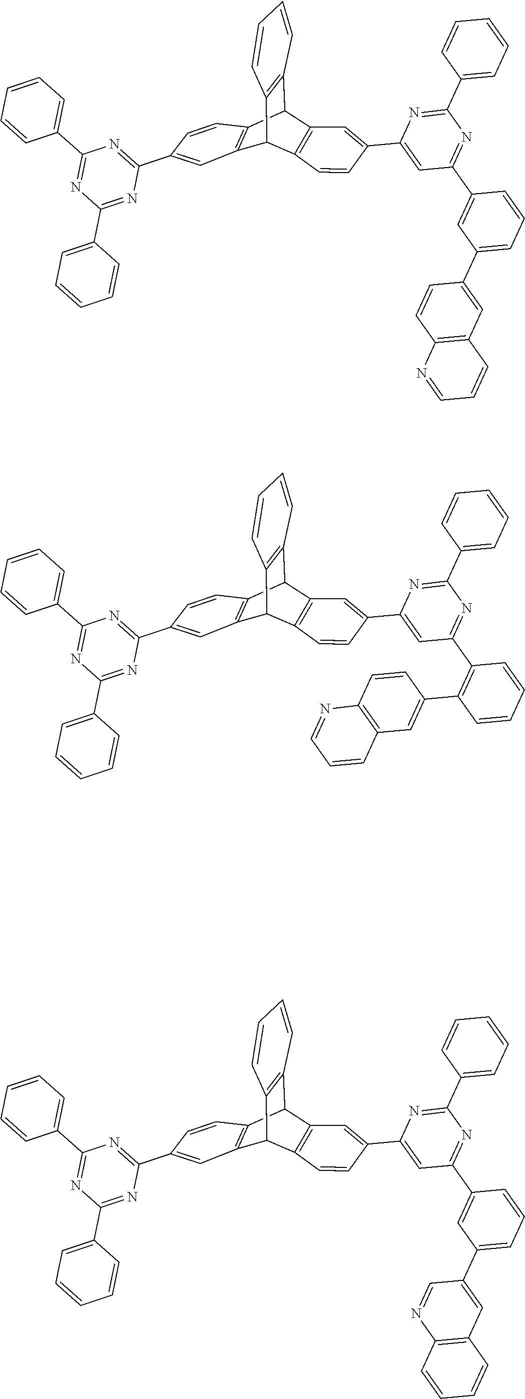 [SMILES: c1ccc(-c2nc(-c3cccc(-c4ccc5ncccc5c4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4ncccc4c3)n2)cc1 ; c1ccc(-c2nc(-c3cccc(-c4cnc5ccccc5c4)c3)cc(-c3ccc4c(c3)C3c5ccccc5C4c4cc(-c5nc(-c6ccccc6)nc(-c6ccccc6)n5)ccc43)n2)cc1]

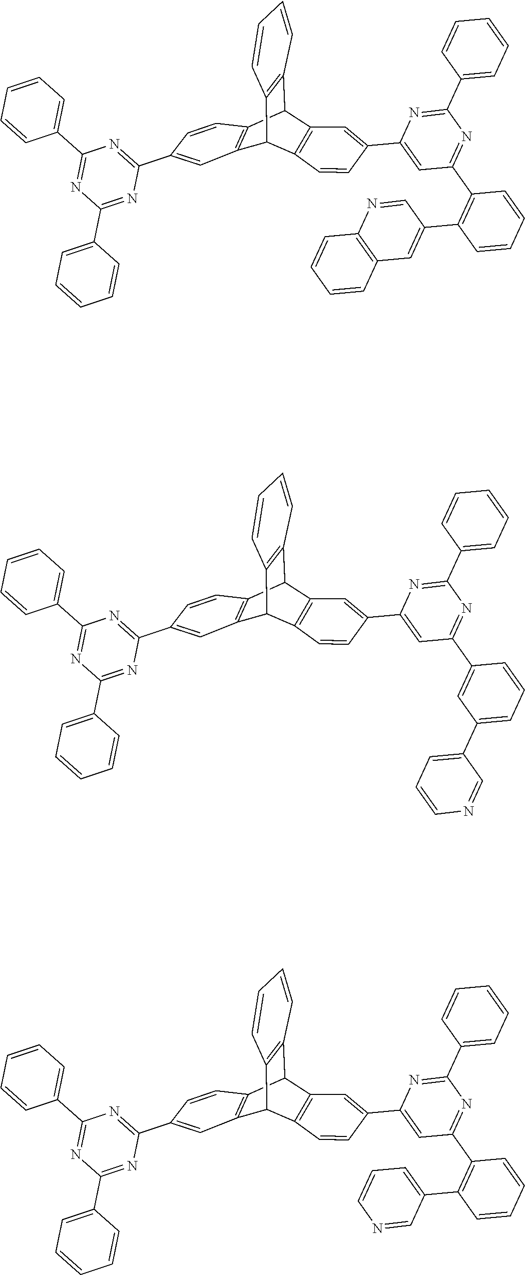 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cnc4ccccc4c3)n2)cc1 ; c1ccc(-c2nc(-c3cccc(-c4cccnc4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccnc3)n2)cc1]

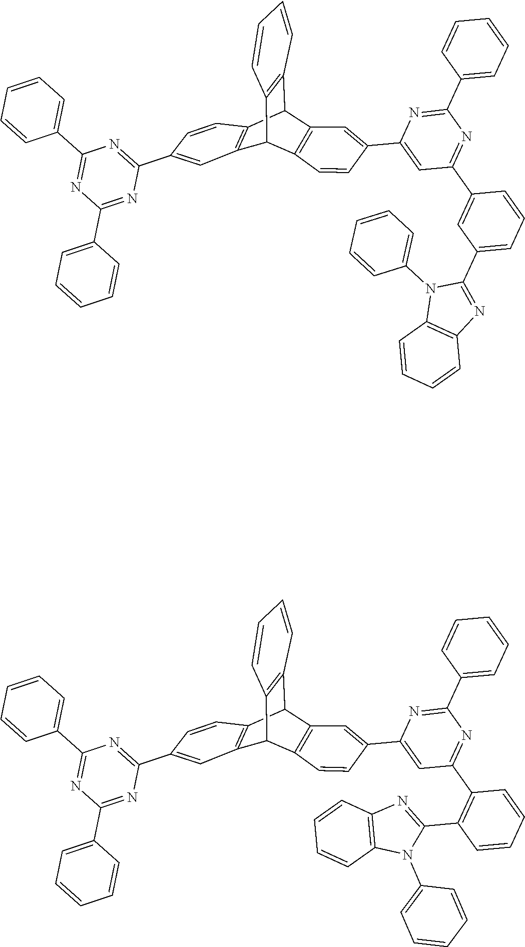 [SMILES: c1ccc(-c2nc(-c3cccc(-c4nc5ccccc5n4-c4ccccc4)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3nc4ccccc4n3-c3ccccc3)n2)cc1]

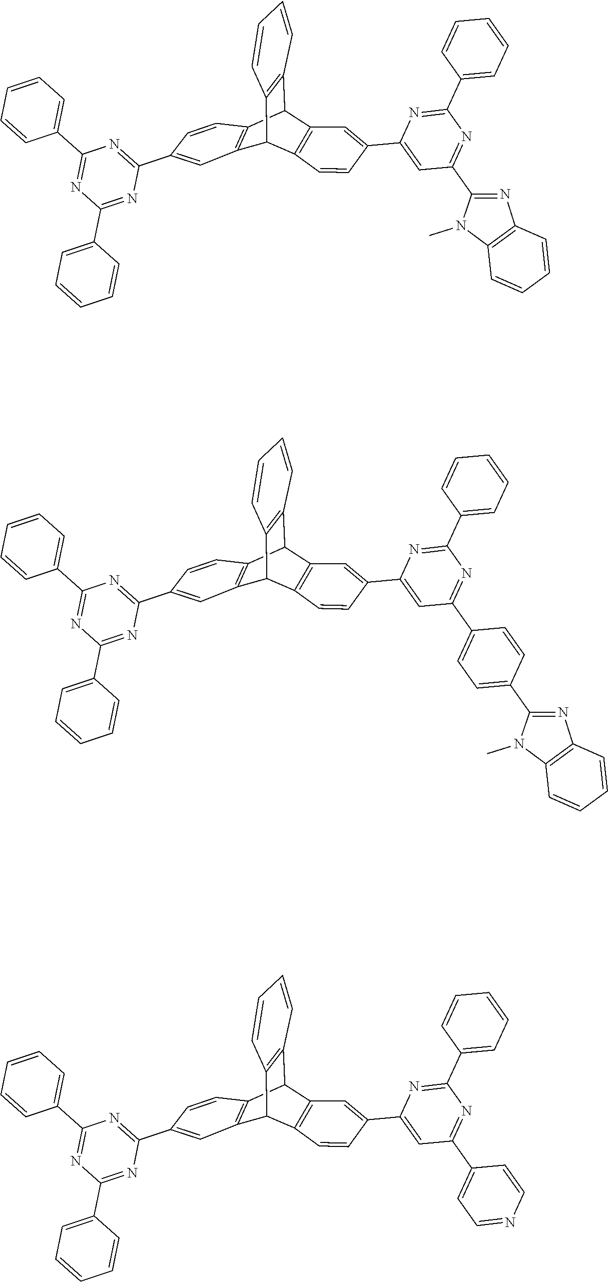 [SMILES: Cn1c(-c2cc(-c3ccc4c(c3)C3c5ccccc5C4c4cc(-c5nc(-c6ccccc6)nc(-c6ccccc6)n5)ccc43)nc(-c3ccccc3)n2)nc2ccccc21 ; Cn1c(-c2ccc(-c3cc(-c4ccc5c(c4)C4c6ccccc6C5c5cc(-c6nc(-c7ccccc7)nc(-c7ccccc7)n6)ccc54)nc(-c4ccccc4)n3)cc2)nc2ccccc21 ; c1ccc(-c2nc(-c3ccncc3)cc(-c3ccc4c(c3)C3c5ccccc5C4c4cc(-c5nc(-c6ccccc6)nc(-c6ccccc6)n5)ccc43)n2)cc1]

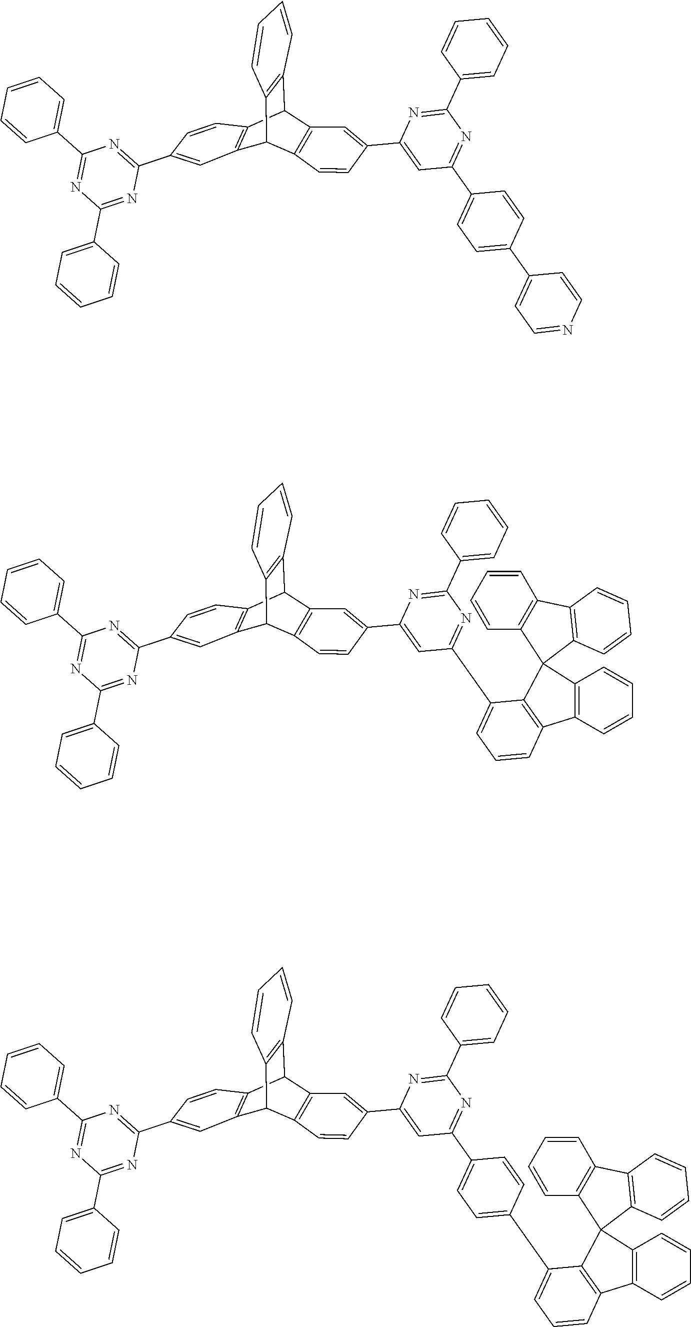 [SMILES: c1ccc(-c2nc(-c3ccc(-c4ccncc4)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3cccc4c3C3(c5ccccc5-c5ccccc53)c3ccccc3-4)n2)cc1 ; c1ccc(-c2nc(-c3ccc(-c4cccc5c4C4(c6ccccc6-c6ccccc64)c4ccccc4-5)cc3)cc(-c3ccc4c(c3)C3c5ccccc5C4c4cc(-c5nc(-c6ccccc6)nc(-c6ccccc6)n5)ccc43)n2)cc1]

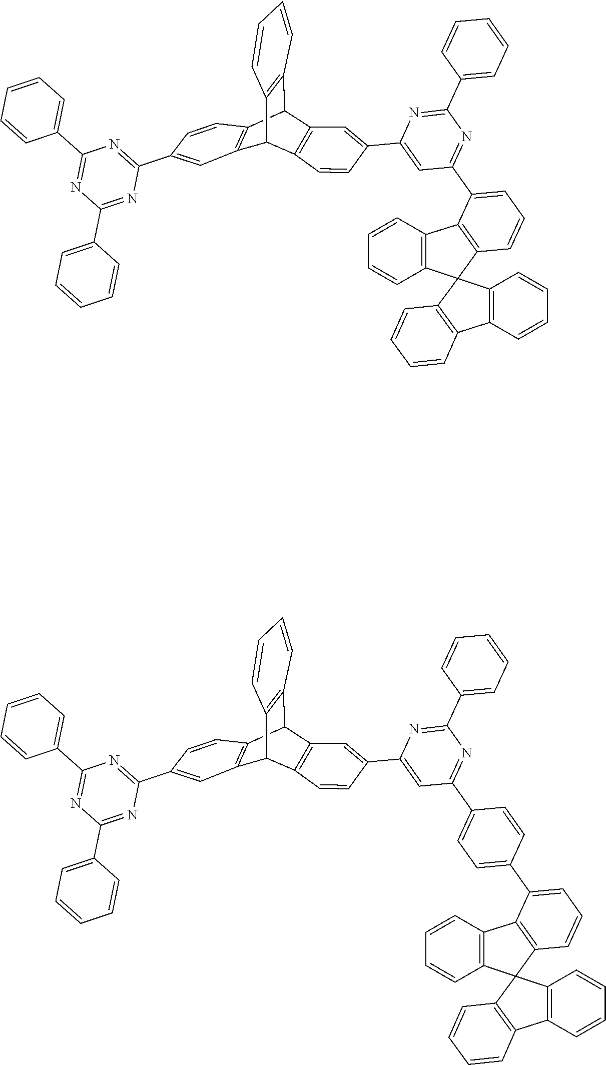 [SMILES: c1ccc(-c2nc(-c3ccc4c(c3)C3c5ccccc5C4c4cc(-c5nc(-c6ccccc6)nc(-c6ccccc6)n5)ccc43)cc(-c3cccc4c3-c3ccccc3C43c4ccccc4-c4ccccc43)n2)cc1 ; c1ccc(-c2nc(-c3ccc(-c4cccc5c4-c4ccccc4C54c5ccccc5-c5ccccc54)cc3)cc(-c3ccc4c(c3)C3c5ccccc5C4c4cc(-c5nc(-c6ccccc6)nc(-c6ccccc6)n5)ccc43)n2)cc1]

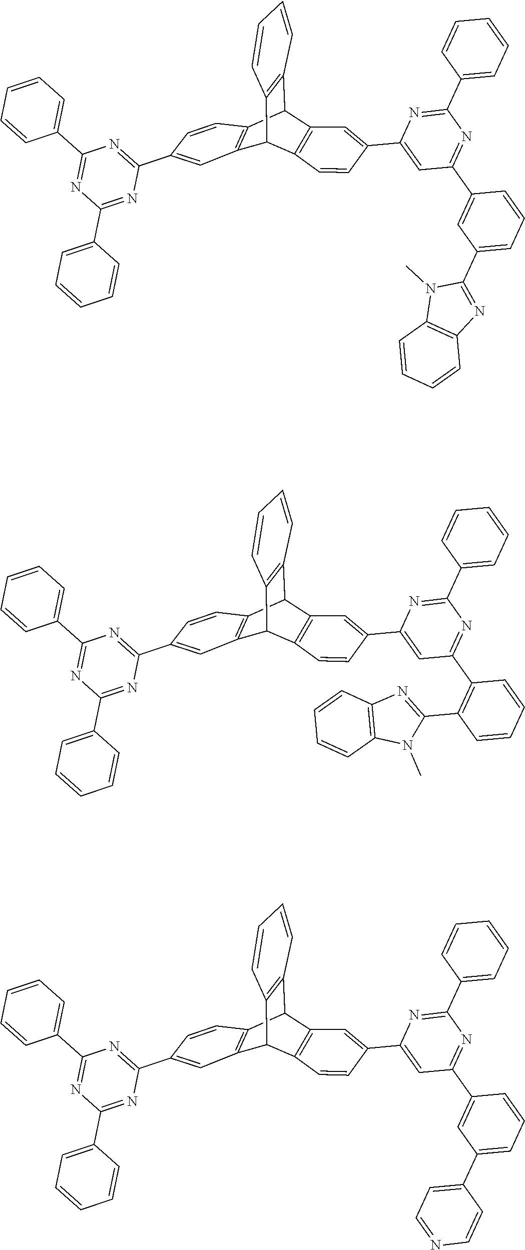 [SMILES: Cn1c(-c2cccc(-c3cc(-c4ccc5c(c4)C4c6ccccc6C5c5cc(-c6nc(-c7ccccc7)nc(-c7ccccc7)n6)ccc54)nc(-c4ccccc4)n3)c2)nc2ccccc21 ; Cn1c(-c2ccccc2-c2cc(-c3ccc4c(c3)C3c5ccccc5C4c4cc(-c5nc(-c6ccccc6)nc(-c6ccccc6)n5)ccc43)nc(-c3ccccc3)n2)nc2ccccc21 ; c1ccc(-c2nc(-c3cccc(-c4ccncc4)c3)cc(-c3ccc4c(c3)C3c5ccccc5C4c4cc(-c5nc(-c6ccccc6)nc(-c6ccccc6)n5)ccc43)n2)cc1]

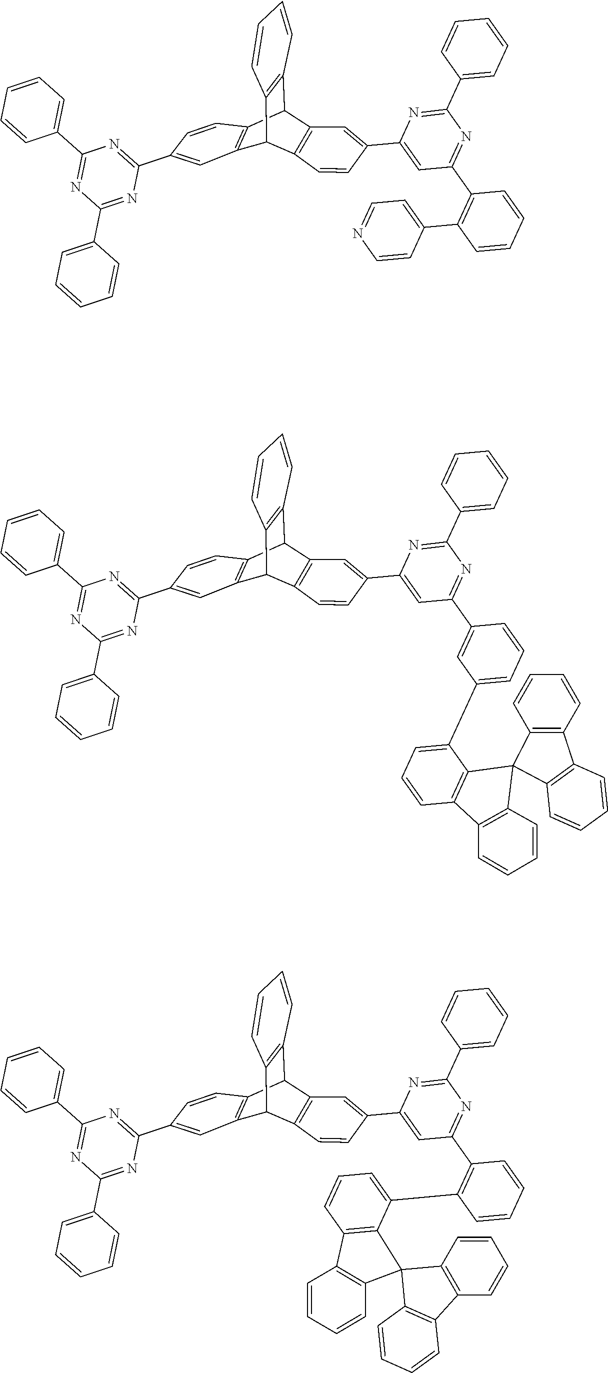 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccncc3)n2)cc1 ; c1ccc(-c2nc(-c3cccc(-c4cccc5c4C4(c6ccccc6-c6ccccc64)c4ccccc4-5)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccc4c3C3(c5ccccc5-c5ccccc53)c3ccccc3-4)n2)cc1]

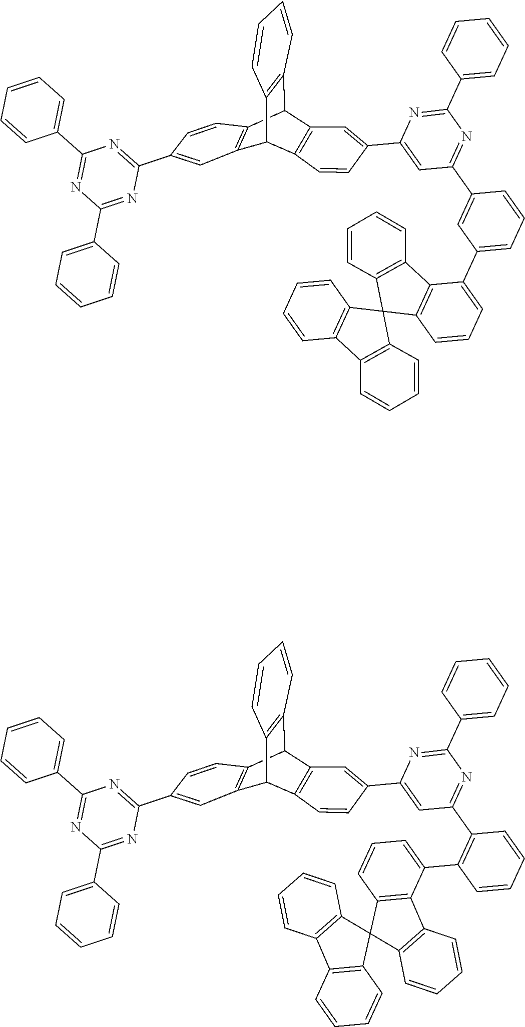 [SMILES: c1ccc(-c2nc(-c3cccc(-c4cccc5c4-c4ccccc4C54c5ccccc5-c5ccccc54)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3cccc4c3-c3ccccc3C43c4ccccc4-c4ccccc43)n2)cc1]

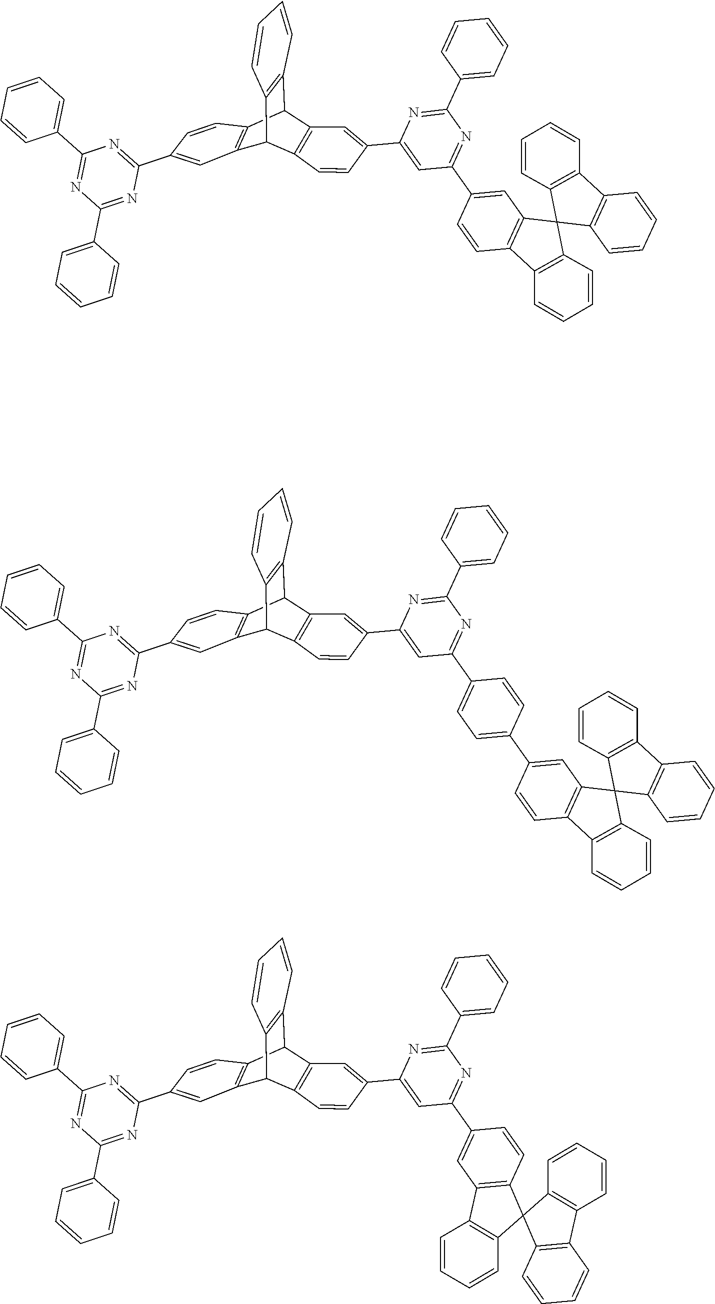 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)C3(c5ccccc5-c5ccccc53)c3ccccc3-4)n2)cc1 ; c1ccc(-c2nc(-c3ccc(-c4ccc5c(c4)C4(c6ccccc6-c6ccccc64)c4ccccc4-5)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)-c3ccccc3C43c4ccccc4-c4ccccc43)cc(-c3ccc4c(c3)C3c5ccccc5C4c4cc(-c5nc(-c6ccccc6)nc(-c6ccccc6)n5)ccc43)n2)cc1]

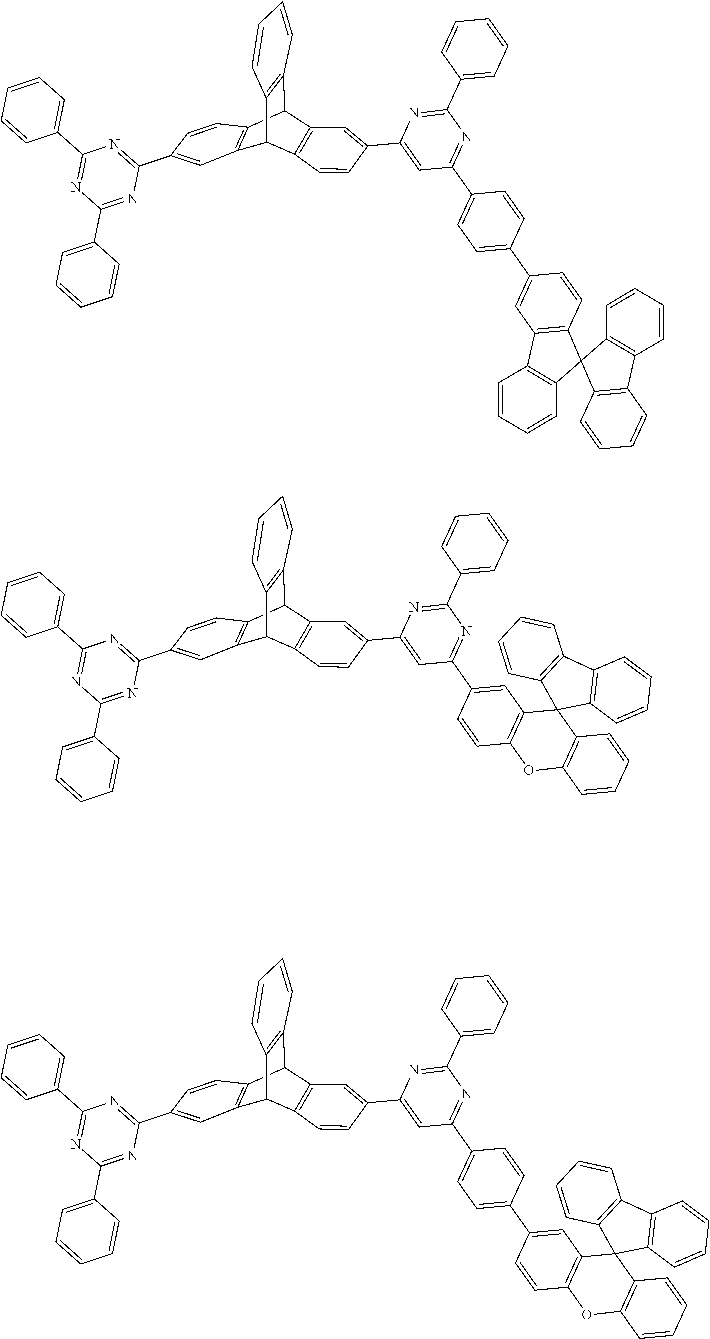 [SMILES: c1ccc(-c2nc(-c3ccc(-c4ccc5c(c4)-c4ccccc4C54c5ccccc5-c5ccccc54)cc3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)C3(c5ccccc5O4)c4ccccc4-c4ccccc43)n2)cc1 ; c1ccc(-c2nc(-c3ccc(-c4ccc5c(c4)C4(c6ccccc6O5)c5ccccc5-c5ccccc54)cc3)cc(-c3ccc4c(c3)C3c5ccccc5C4c4cc(-c5nc(-c6ccccc6)nc(-c6ccccc6)n5)ccc43)n2)cc1]

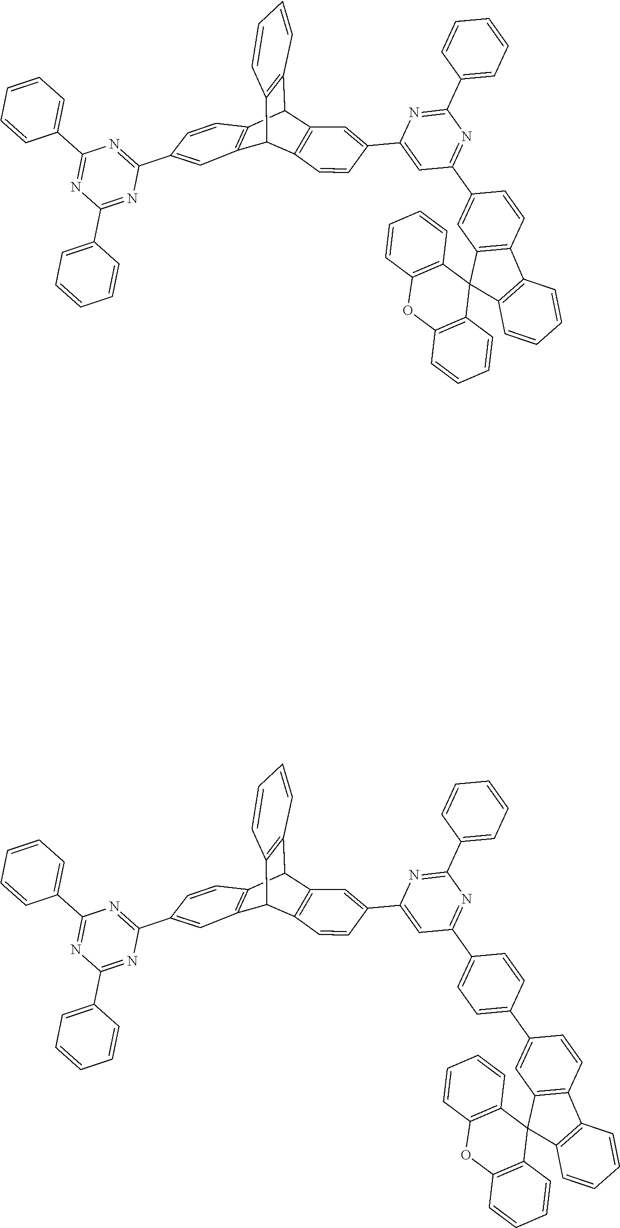 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccc4c(c3)C3(c5ccccc5Oc5ccccc53)c3ccccc3-4)n2)cc1 ; c1ccc(-c2nc(-c3ccc(-c4ccc5c(c4)C4(c6ccccc6Oc6ccccc64)c4ccccc4-5)cc3)cc(-c3ccc4c(c3)C3c5ccccc5C4c4cc(-c5nc(-c6ccccc6)nc(-c6ccccc6)n5)ccc43)n2)cc1]

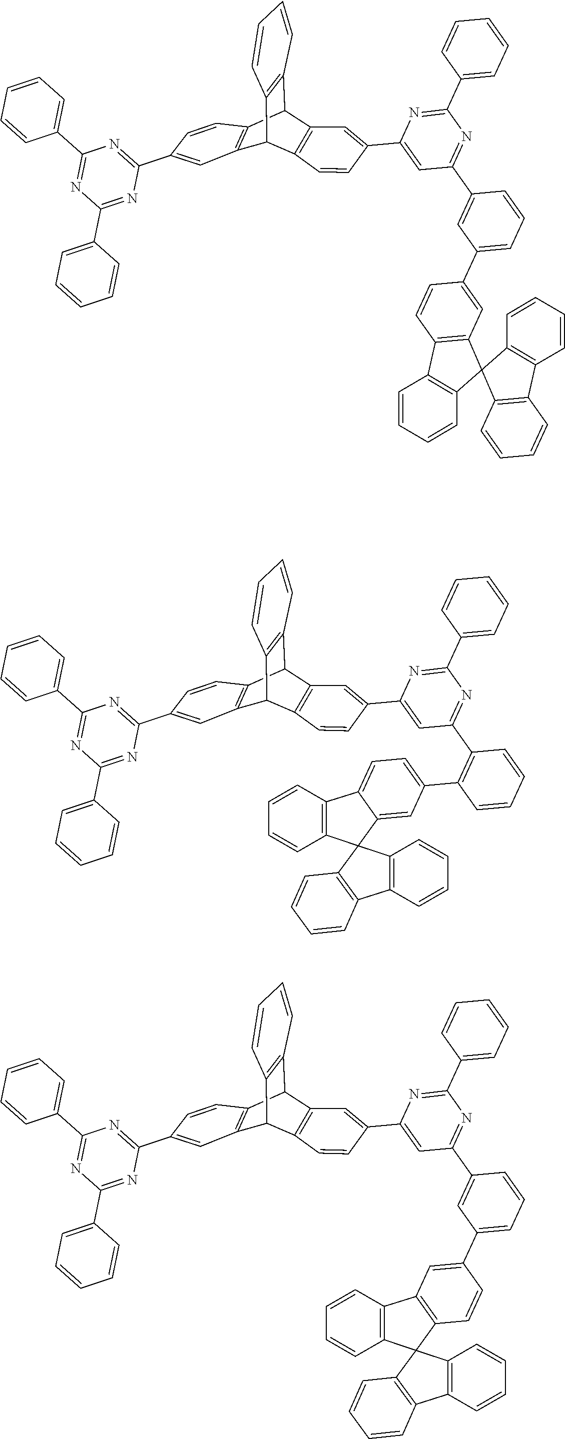 [SMILES: c1ccc(-c2nc(-c3cccc(-c4ccc5c(c4)C4(c6ccccc6-c6ccccc64)c4ccccc4-5)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)C3(c5ccccc5-c5ccccc53)c3ccccc3-4)n2)cc1 ; c1ccc(-c2nc(-c3cccc(-c4ccc5c(c4)-c4ccccc4C54c5ccccc5-c5ccccc54)c3)cc(-c3ccc4c(c3)C3c5ccccc5C4c4cc(-c5nc(-c6ccccc6)nc(-c6ccccc6)n5)ccc43)n2)cc1]

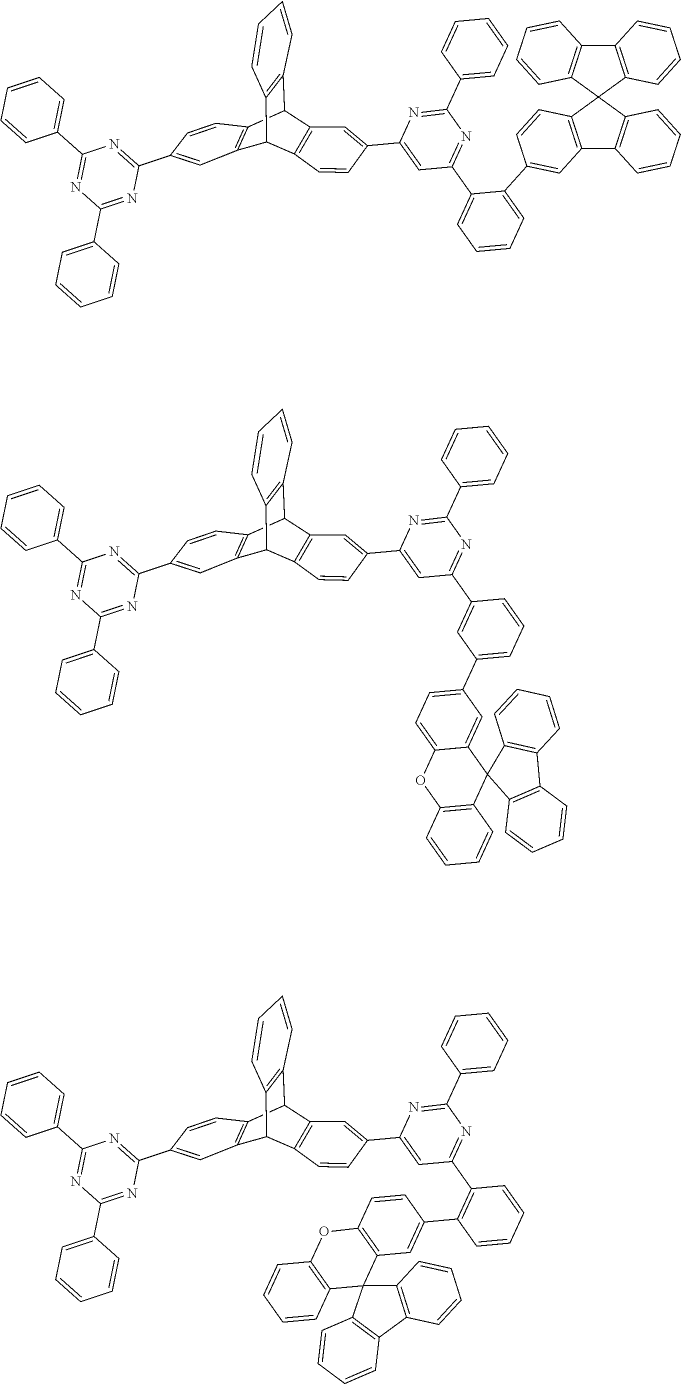 [SMILES: c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)-c3ccccc3C43c4ccccc4-c4ccccc43)n2)cc1 ; c1ccc(-c2nc(-c3cccc(-c4ccc5c(c4)C4(c6ccccc6O5)c5ccccc5-c5ccccc54)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)C3(c5ccccc5O4)c4ccccc4-c4ccccc43)n2)cc1]

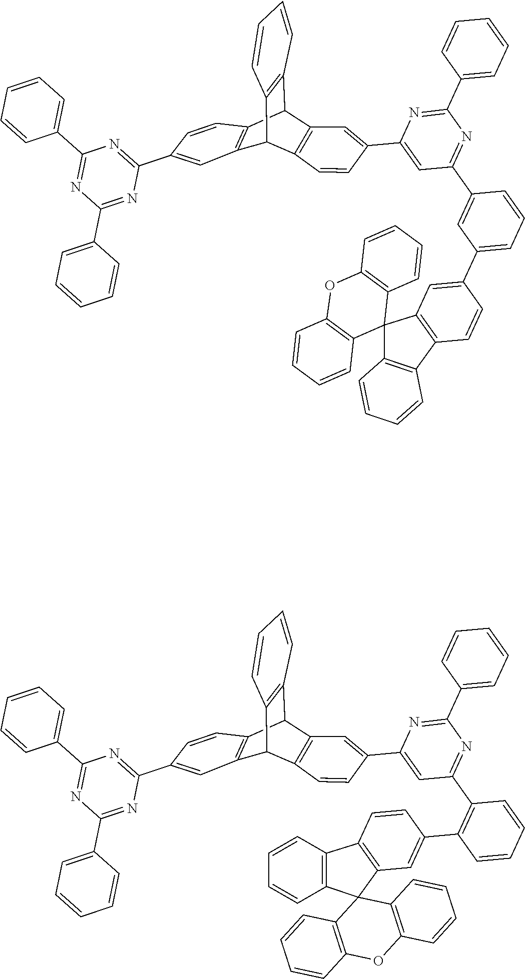 [SMILES: c1ccc(-c2nc(-c3cccc(-c4ccc5c(c4)C4(c6ccccc6Oc6ccccc64)c4ccccc4-5)c3)cc(-c3ccc4c(c3)C3c5ccccc5C4c4cc(-c5nc(-c6ccccc6)nc(-c6ccccc6)n5)ccc43)n2)cc1 ; c1ccc(-c2nc(-c3ccc4c(c3)C3c5ccccc5C4c4cc(-c5nc(-c6ccccc6)nc(-c6ccccc6)n5)ccc43)cc(-c3ccccc3-c3ccc4c(c3)C3(c5ccccc5Oc5ccccc53)c3ccccc3-4)n2)cc1]

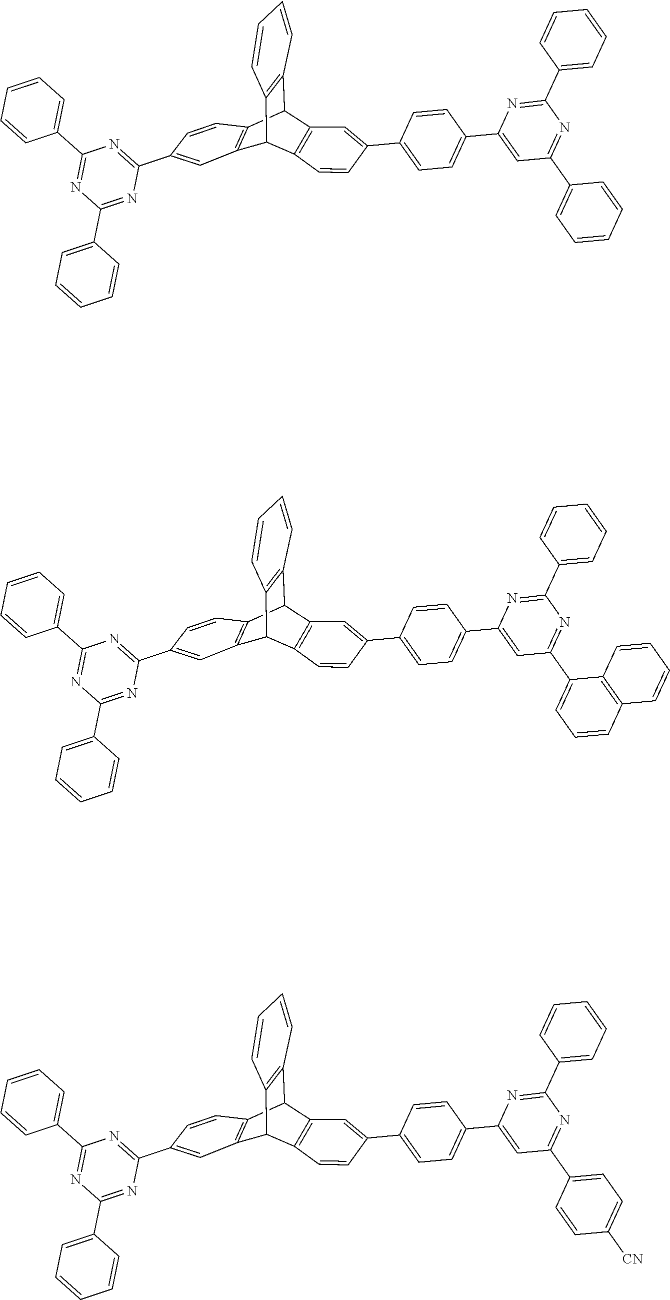 [SMILES: c1ccc(-c2cc(-c3ccc(-c4ccc5c(c4)C4c6ccccc6C5c5cc(-c6nc(-c7ccccc7)nc(-c7ccccc7)n6)ccc54)cc3)nc(-c3ccccc3)n2)cc1 ; c1ccc(-c2nc(-c3ccc(-c4ccc5c(c4)C4c6ccccc6C5c5cc(-c6nc(-c7ccccc7)nc(-c7ccccc7)n6)ccc54)cc3)cc(-c3cccc4ccccc34)n2)cc1 ; N#Cc1ccc(-c2cc(-c3ccc(-c4ccc5c(c4)C4c6ccccc6C5c5cc(-c6nc(-c7ccccc7)nc(-c7ccccc7)n6)ccc54)cc3)nc(-c3ccccc3)n2)cc1]

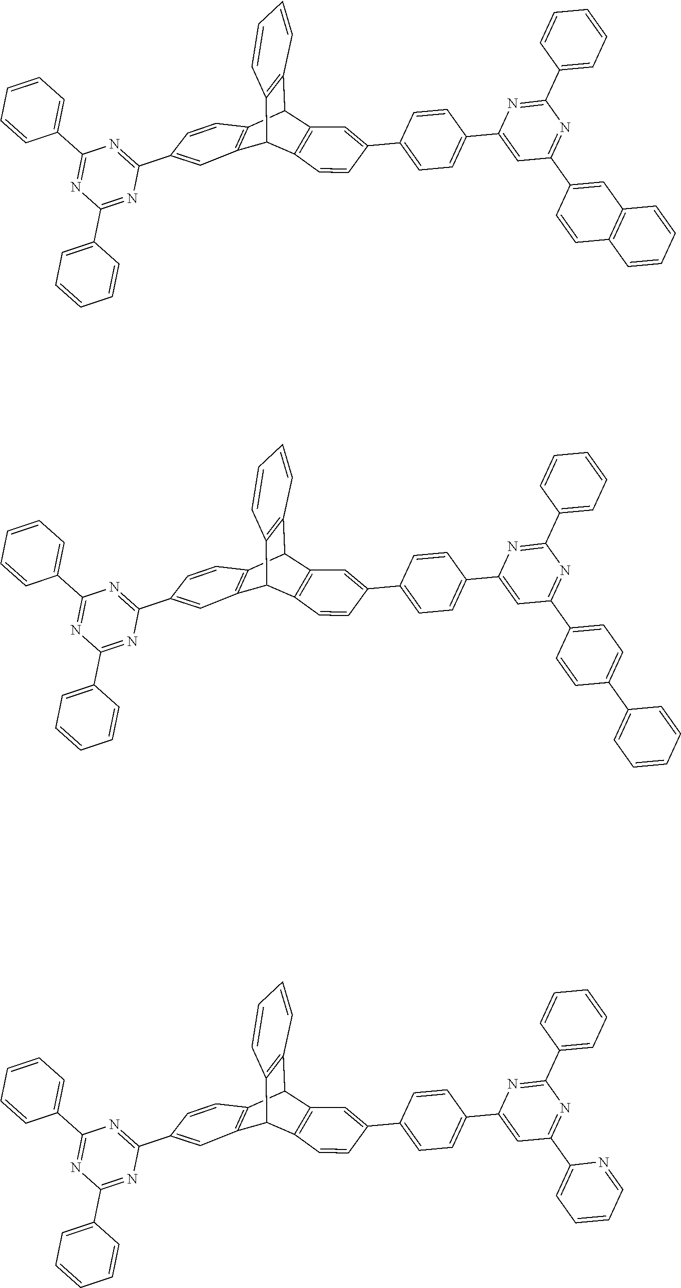 [SMILES: c1ccc(-c2nc(-c3ccc(-c4ccc5c(c4)C4c6ccccc6C5c5cc(-c6nc(-c7ccccc7)nc(-c7ccccc7)n6)ccc54)cc3)cc(-c3ccc4ccccc4c3)n2)cc1 ; c1ccc(-c2ccc(-c3cc(-c4ccc(-c5ccc6c(c5)C5c7ccccc7C6c6cc(-c7nc(-c8ccccc8)nc(-c8ccccc8)n7)ccc65)cc4)nc(-c4ccccc4)n3)cc2)cc1 ; c1ccc(-c2nc(-c3ccc(-c4ccc5c(c4)C4c6ccccc6C5c5cc(-c6nc(-c7ccccc7)nc(-c7ccccc7)n6)ccc54)cc3)cc(-c3ccccn3)n2)cc1]

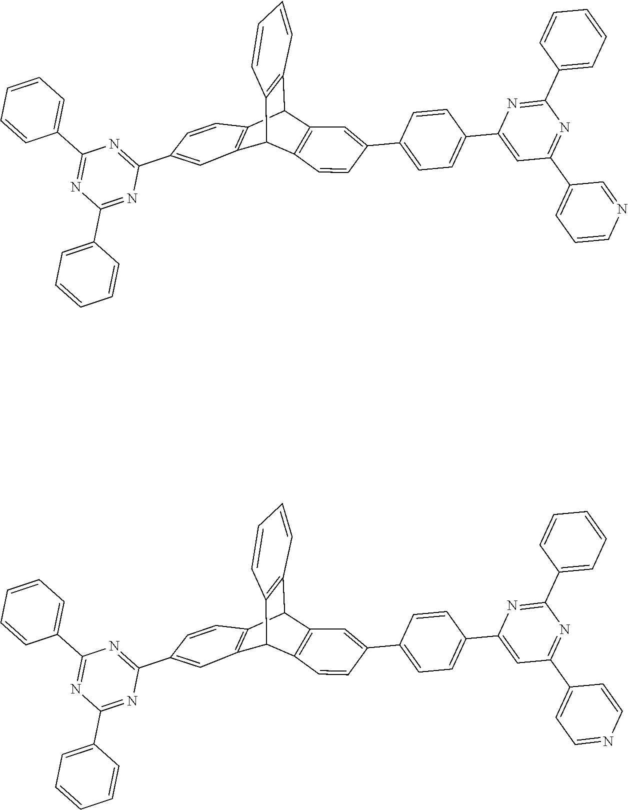 [SMILES: c1ccc(-c2nc(-c3ccc(-c4ccc5c(c4)C4c6ccccc6C5c5cc(-c6nc(-c7ccccc7)nc(-c7ccccc7)n6)ccc54)cc3)cc(-c3cccnc3)n2)cc1 ; c1ccc(-c2nc(-c3ccncc3)cc(-c3ccc(-c4ccc5c(c4)C4c6ccccc6C5c5cc(-c6nc(-c7ccccc7)nc(-c7ccccc7)n6)ccc54)cc3)n2)cc1]

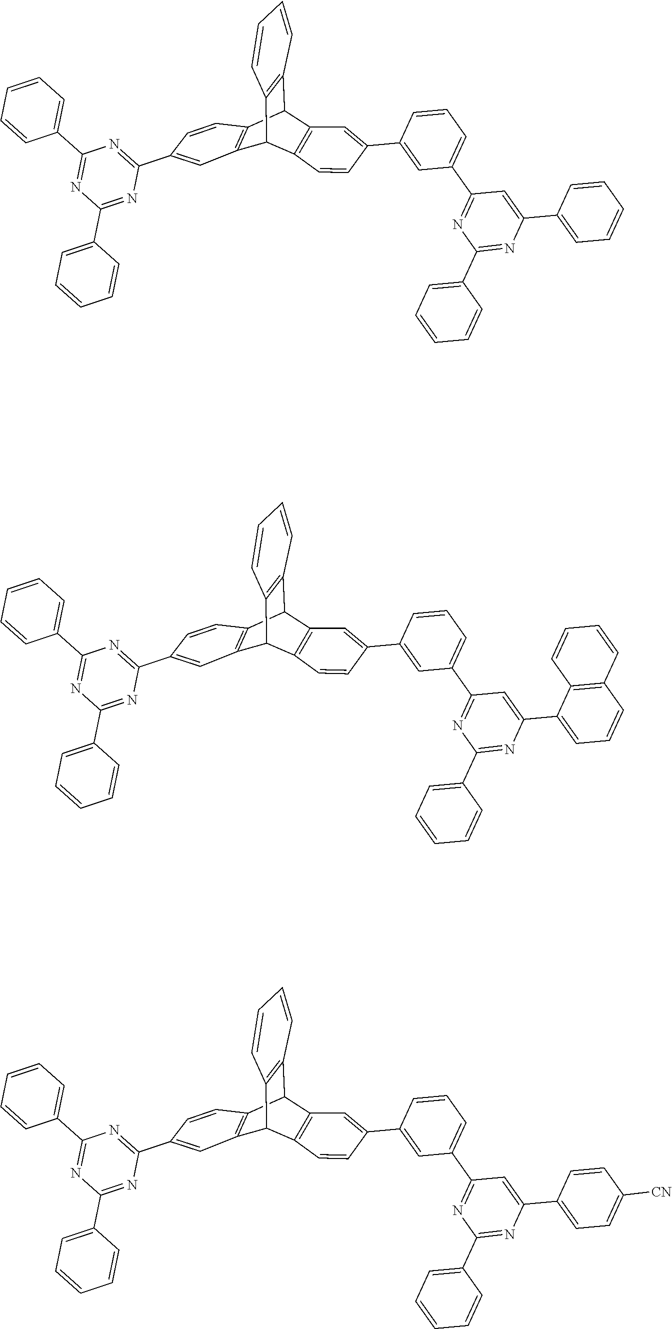 [SMILES: c1ccc(-c2cc(-c3cccc(-c4ccc5c(c4)C4c6ccccc6C5c5cc(-c6nc(-c7ccccc7)nc(-c7ccccc7)n6)ccc54)c3)nc(-c3ccccc3)n2)cc1 ; c1ccc(-c2nc(-c3cccc(-c4ccc5c(c4)C4c6ccccc6C5c5cc(-c6nc(-c7ccccc7)nc(-c7ccccc7)n6)ccc54)c3)cc(-c3cccc4ccccc34)n2)cc1 ; N#Cc1ccc(-c2cc(-c3cccc(-c4ccc5c(c4)C4c6ccccc6C5c5cc(-c6nc(-c7ccccc7)nc(-c7ccccc7)n6)ccc54)c3)nc(-c3ccccc3)n2)cc1]

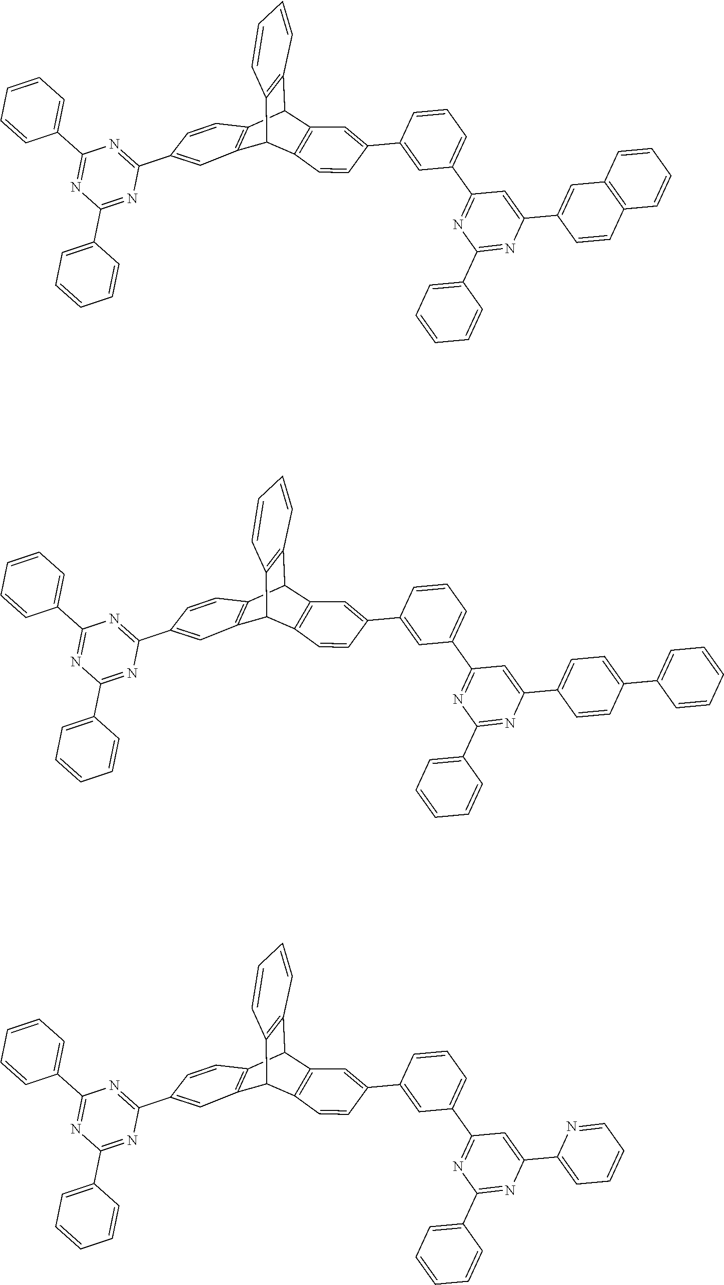 [SMILES: c1ccc(-c2nc(-c3cccc(-c4ccc5c(c4)C4c6ccccc6C5c5cc(-c6nc(-c7ccccc7)nc(-c7ccccc7)n6)ccc54)c3)cc(-c3ccc4ccccc4c3)n2)cc1 ; c1ccc(-c2ccc(-c3cc(-c4cccc(-c5ccc6c(c5)C5c7ccccc7C6c6cc(-c7nc(-c8ccccc8)nc(-c8ccccc8)n7)ccc65)c4)nc(-c4ccccc4)n3)cc2)cc1 ; c1ccc(-c2nc(-c3cccc(-c4ccc5c(c4)C4c6ccccc6C5c5cc(-c6nc(-c7ccccc7)nc(-c7ccccc7)n6)ccc54)c3)cc(-c3ccccn3)n2)cc1]

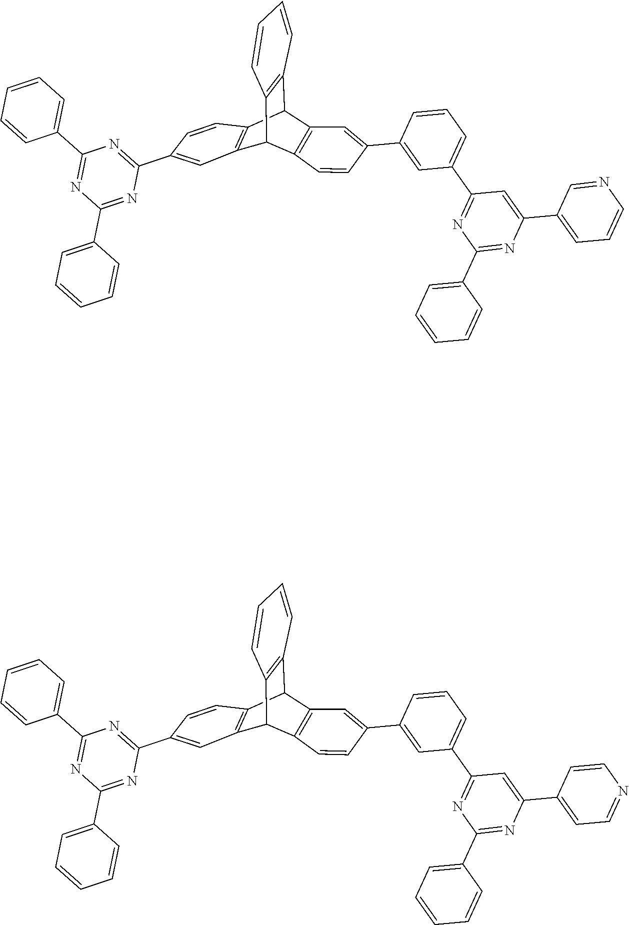 [SMILES: c1ccc(-c2nc(-c3cccnc3)cc(-c3cccc(-c4ccc5c(c4)C4c6ccccc6C5c5cc(-c6nc(-c7ccccc7)nc(-c7ccccc7)n6)ccc54)c3)n2)cc1 ; c1ccc(-c2nc(-c3ccncc3)cc(-c3cccc(-c4ccc5c(c4)C4c6ccccc6C5c5cc(-c6nc(-c7ccccc7)nc(-c7ccccc7)n6)ccc54)c3)n2)cc1]

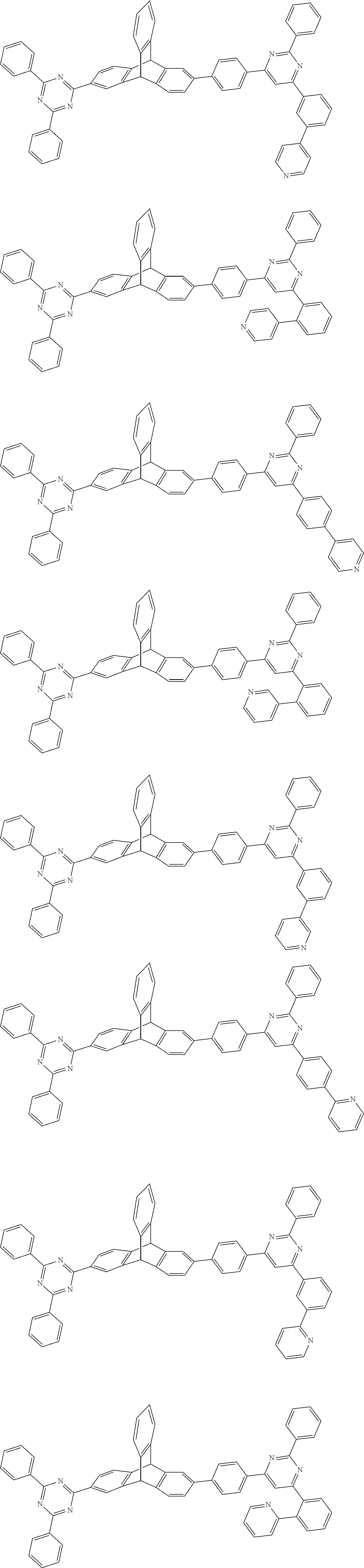

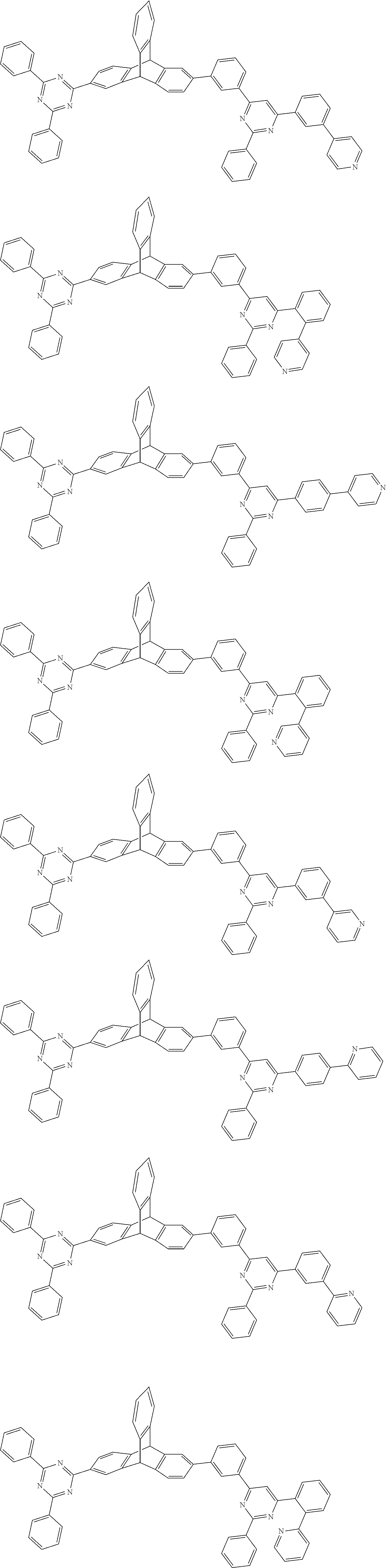

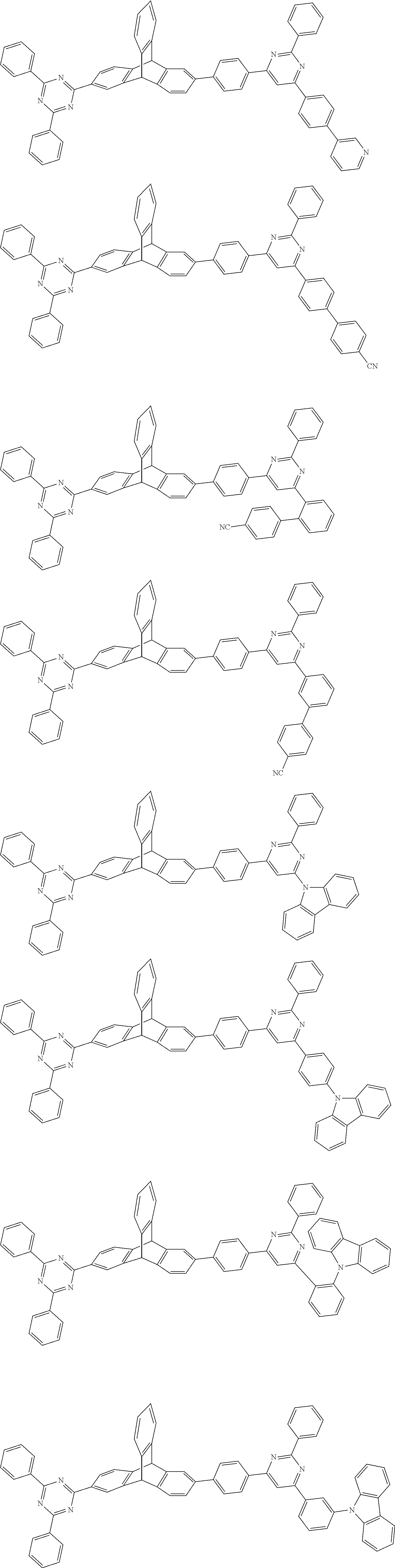

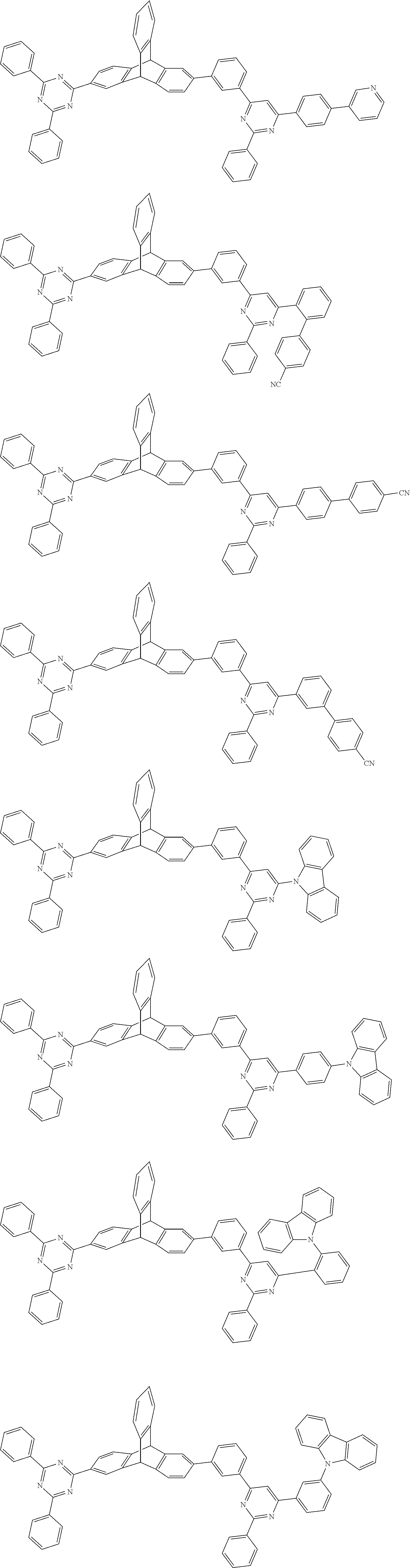

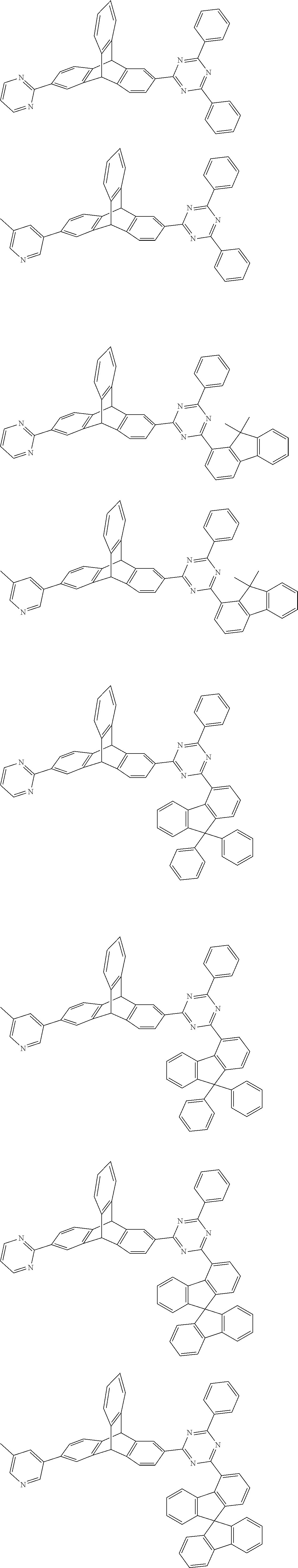

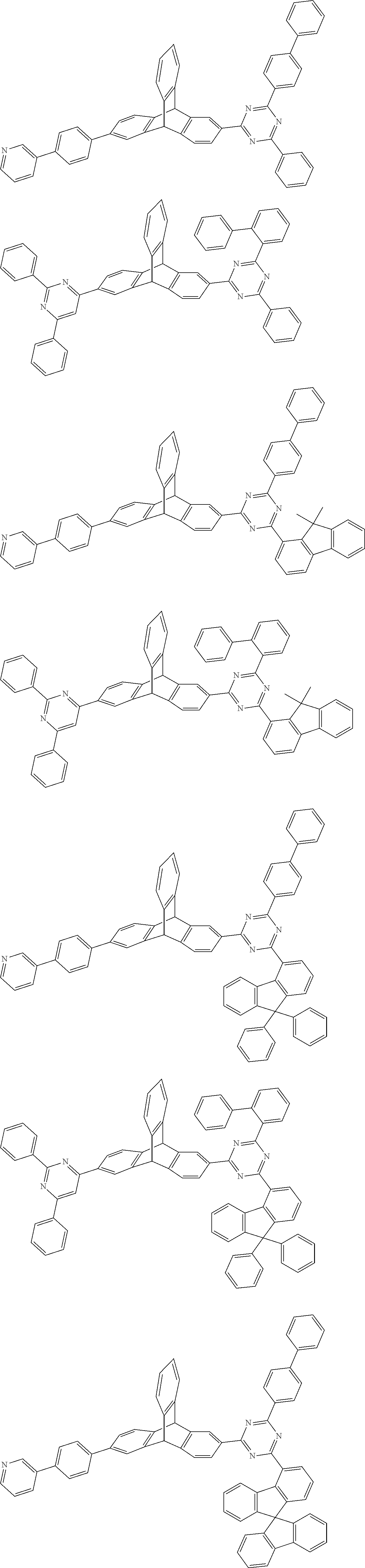

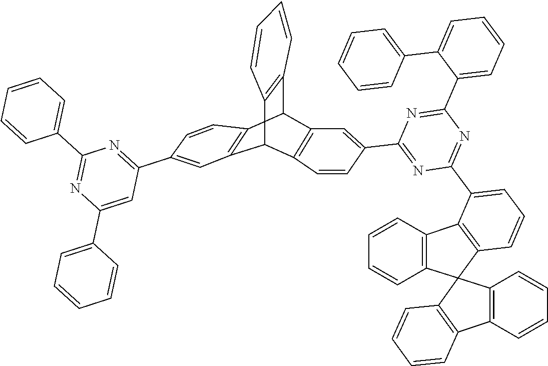 [SMILES: c1ccc(-c2cc(-c3ccc4c(c3)C3c5ccccc5C4c4cc(-c5nc(-c6ccccc6-c6ccccc6)nc(-c6cccc7c6-c6ccccc6C76c7ccccc7-c7ccccc76)n5)ccc43)nc(-c3ccccc3)n2)cc1]

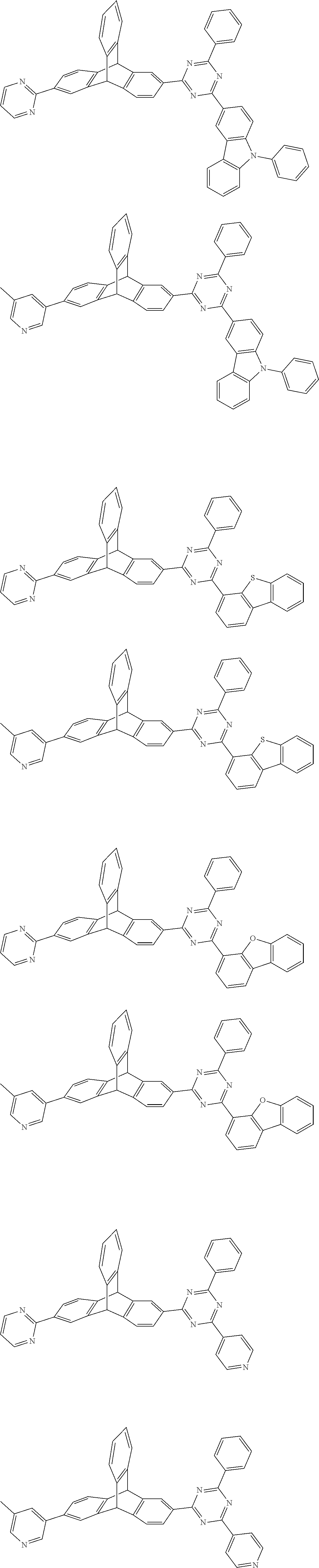

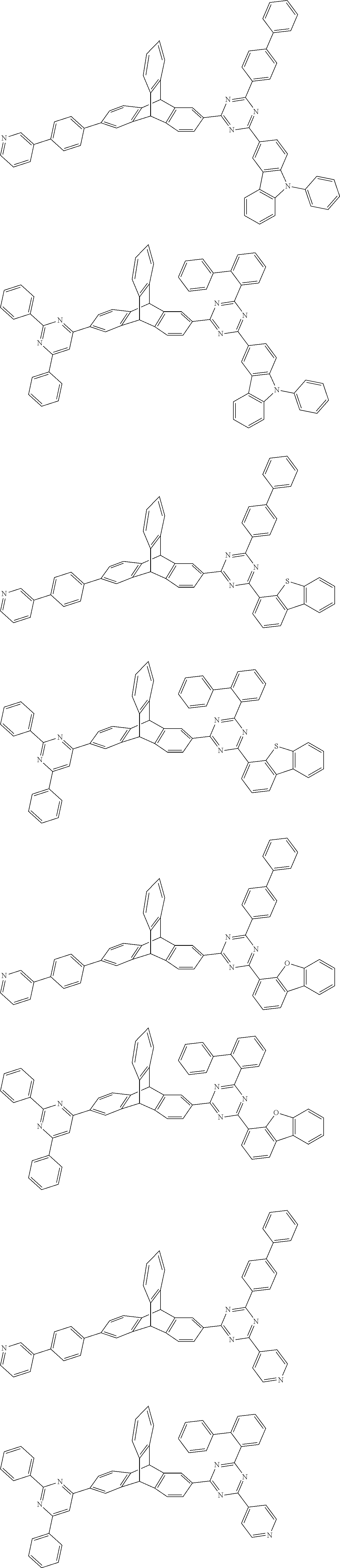

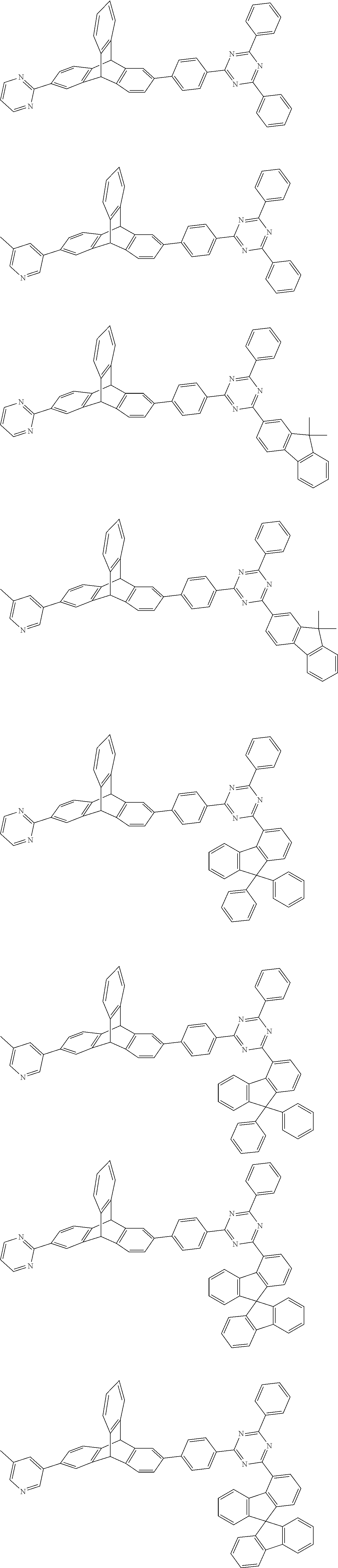

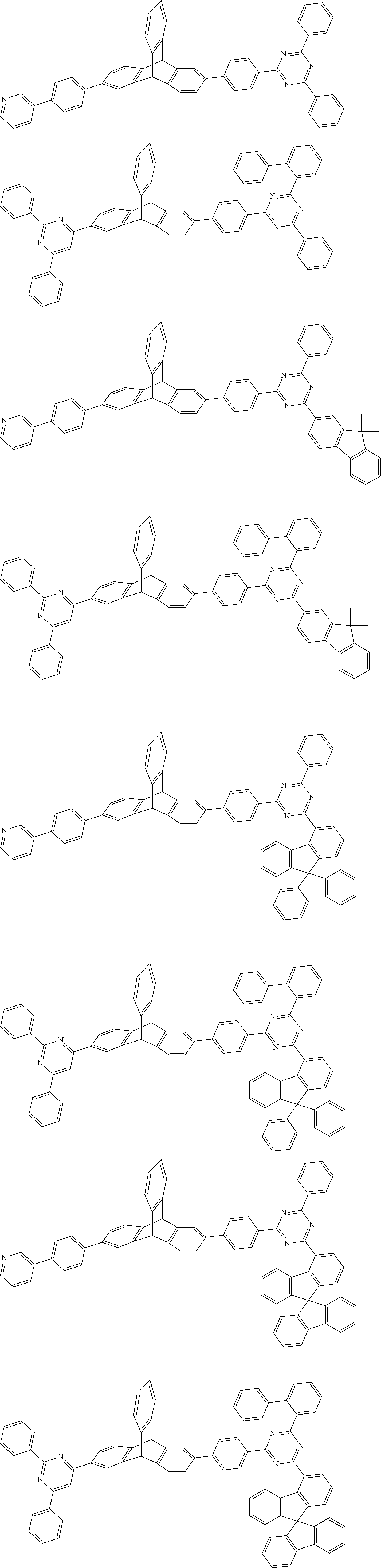

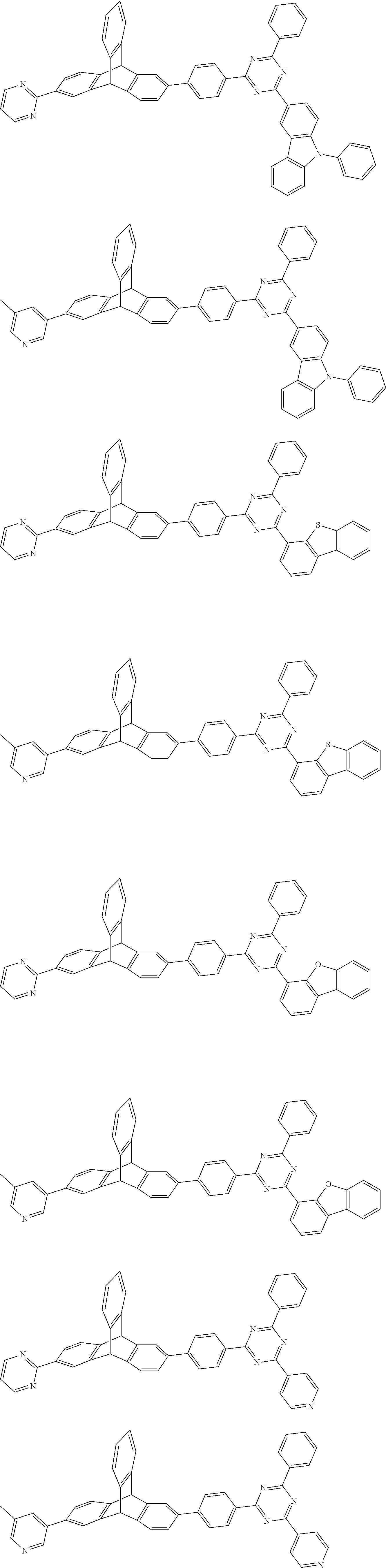

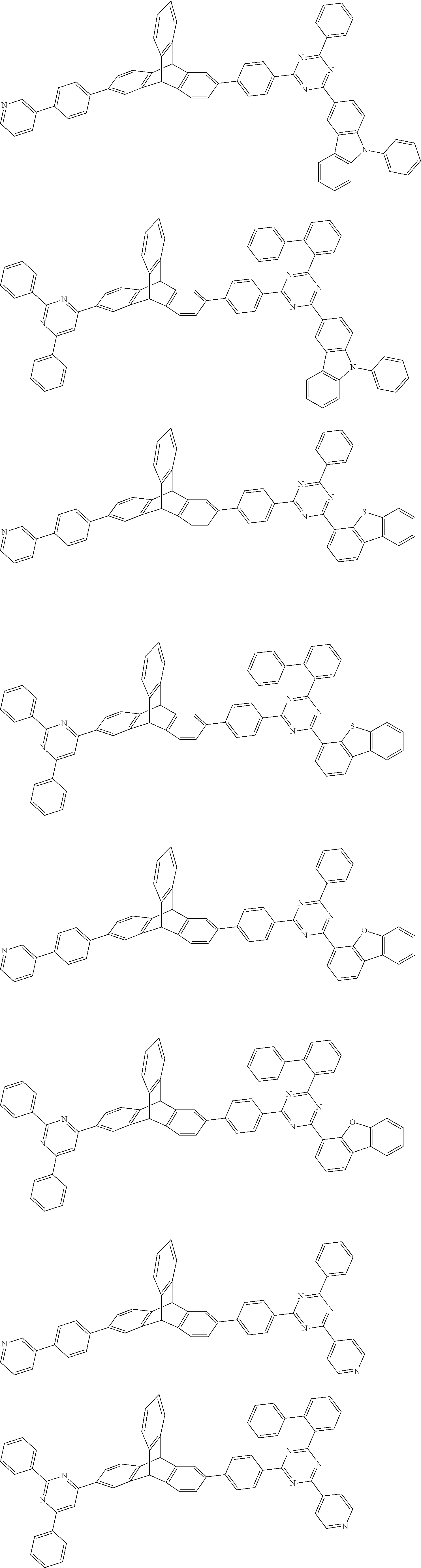

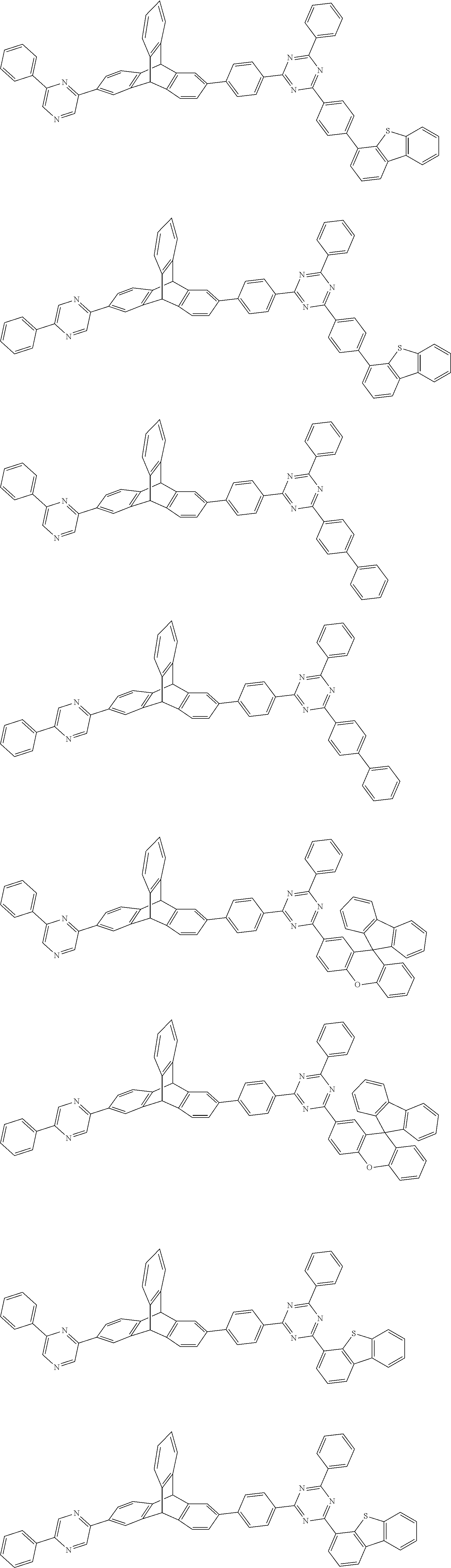

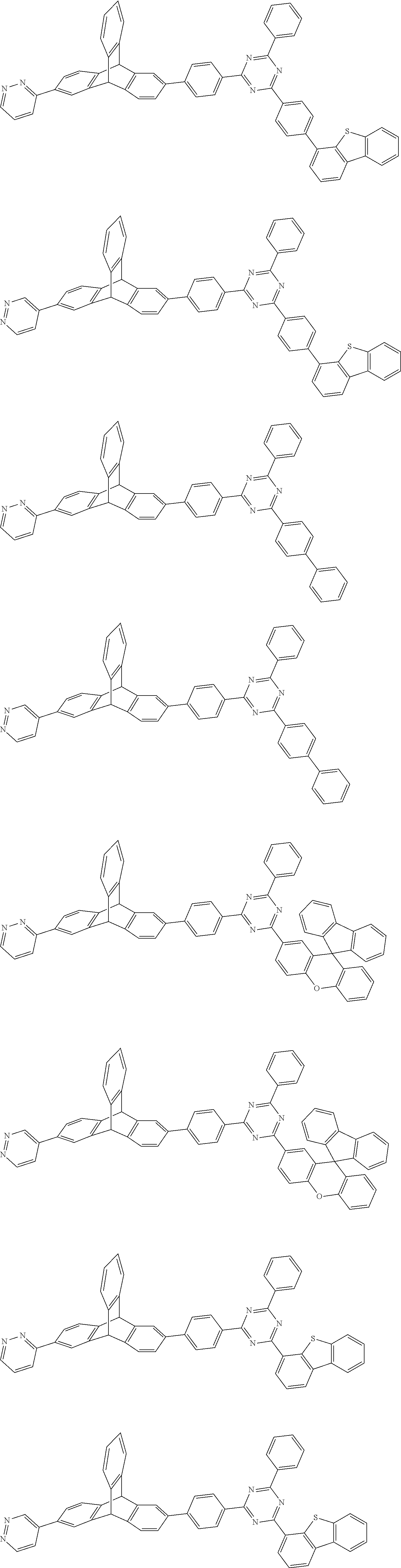

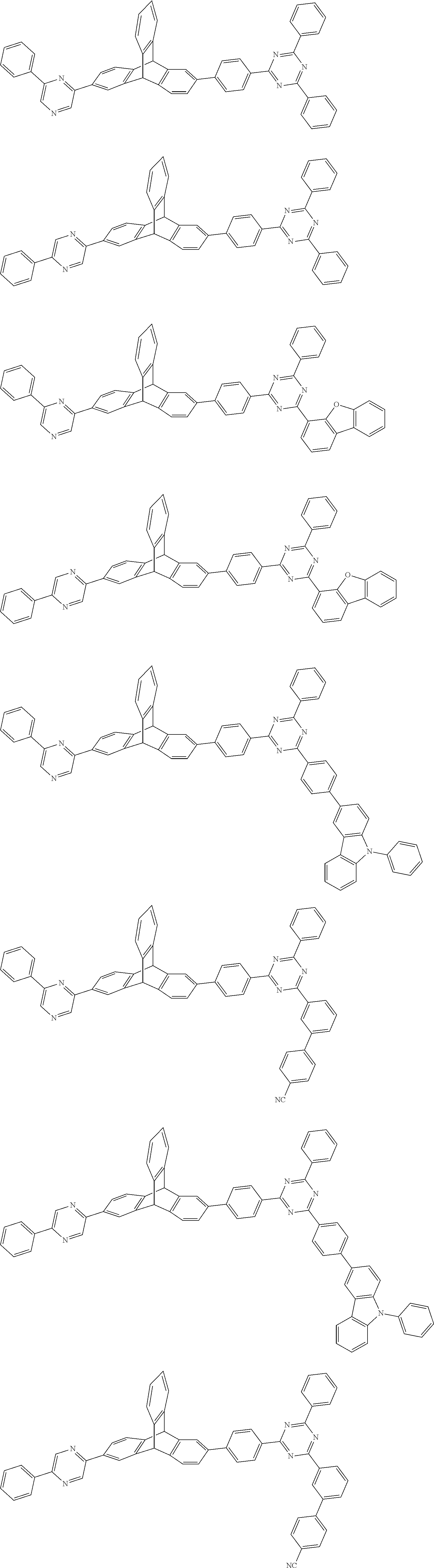

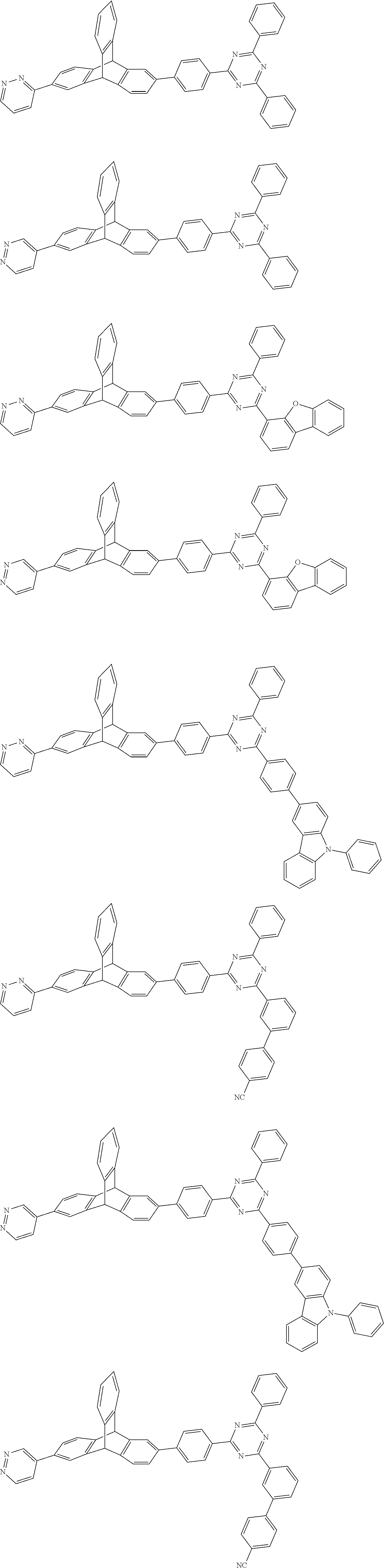

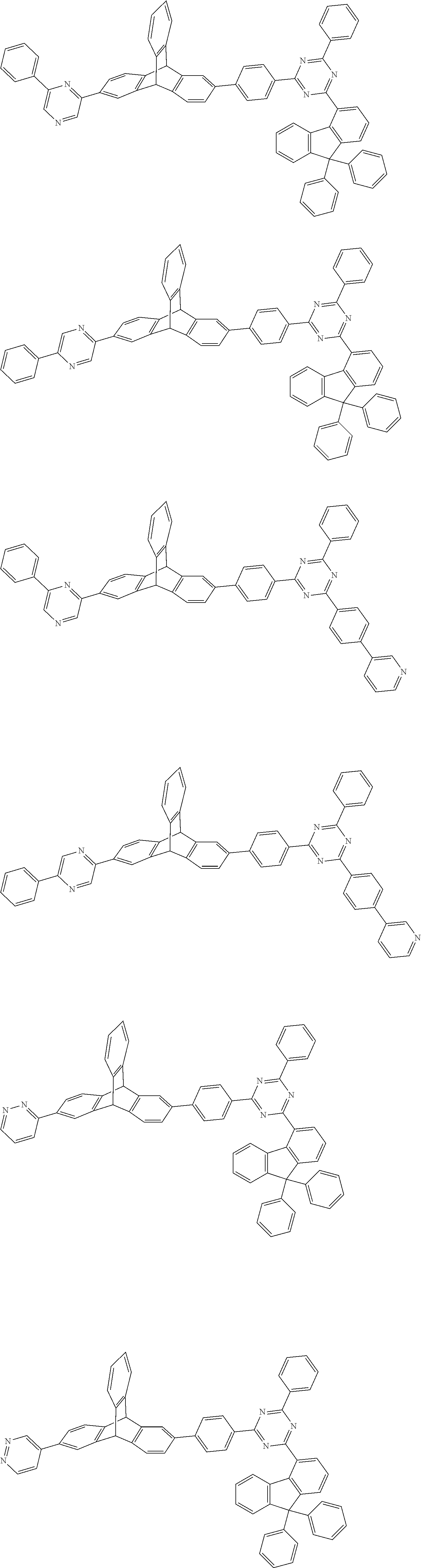

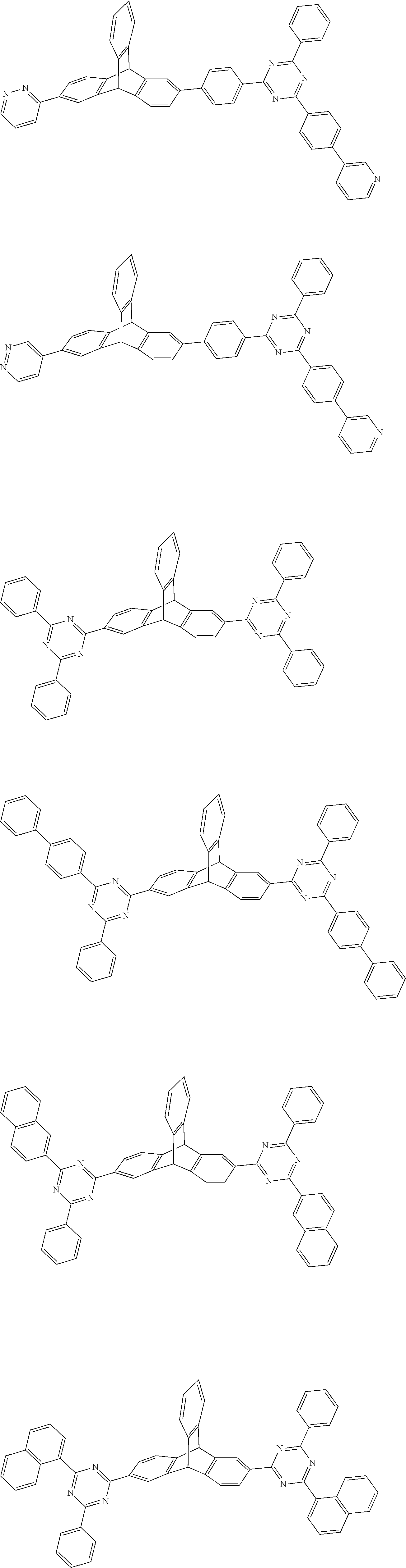

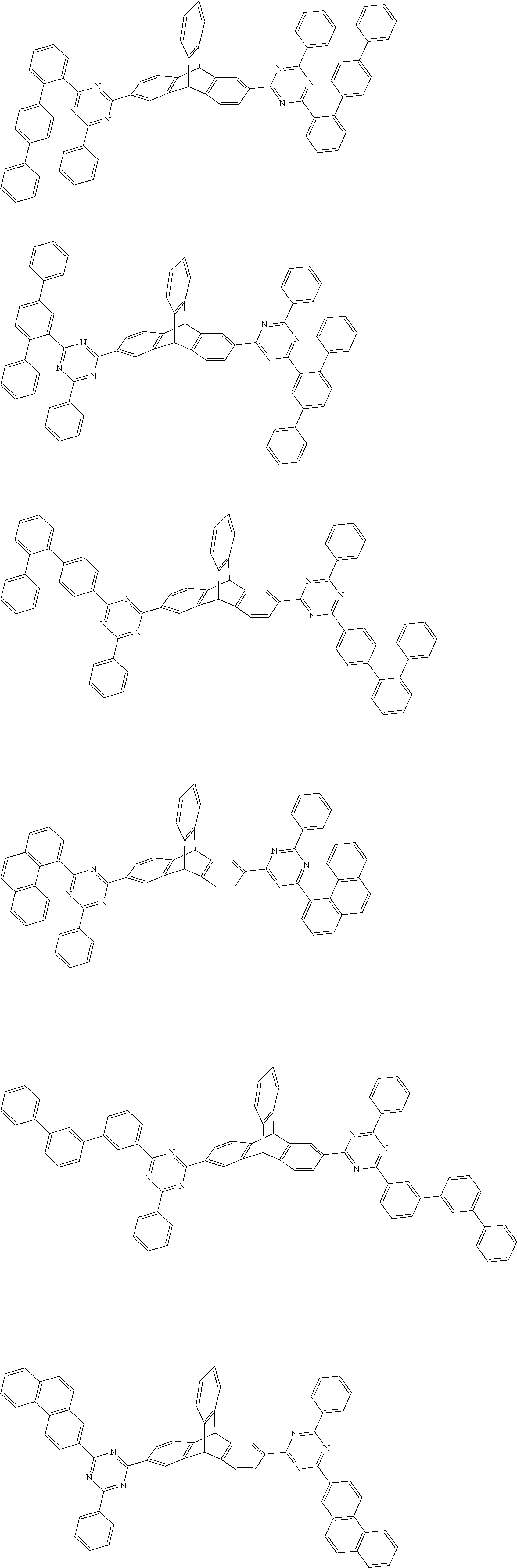

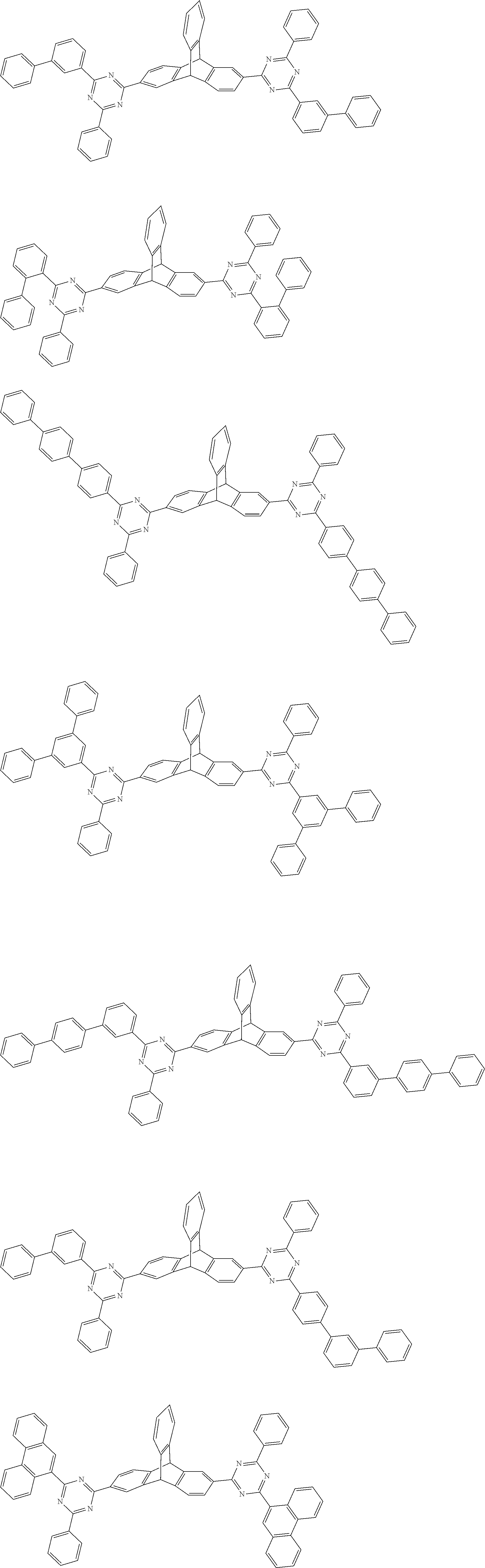

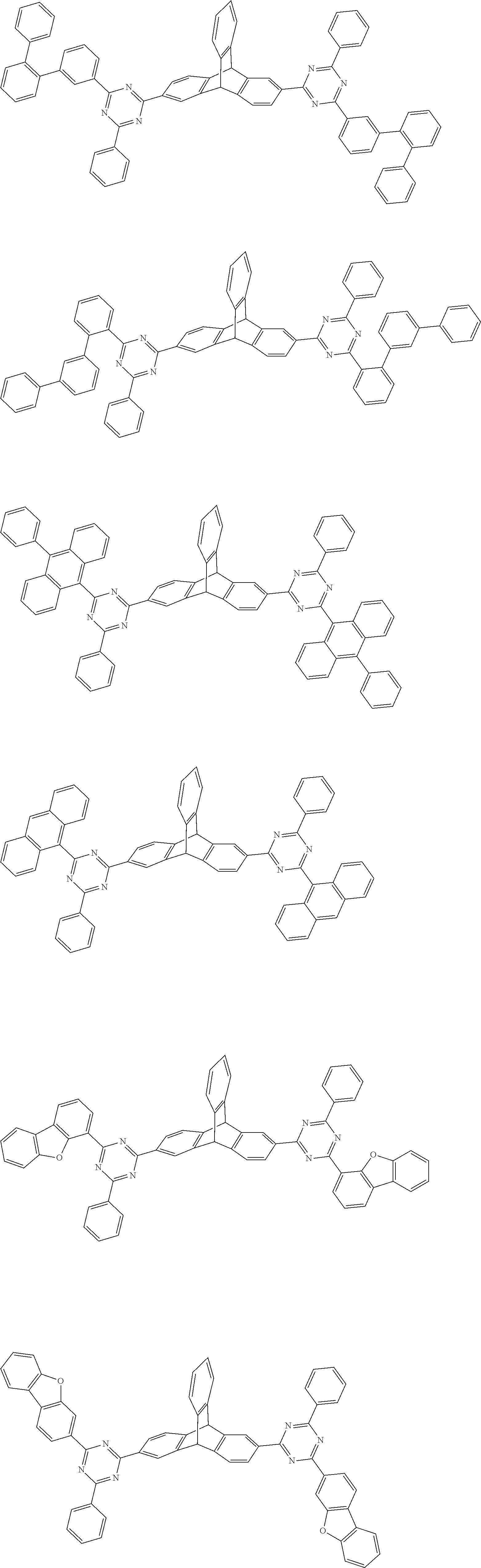

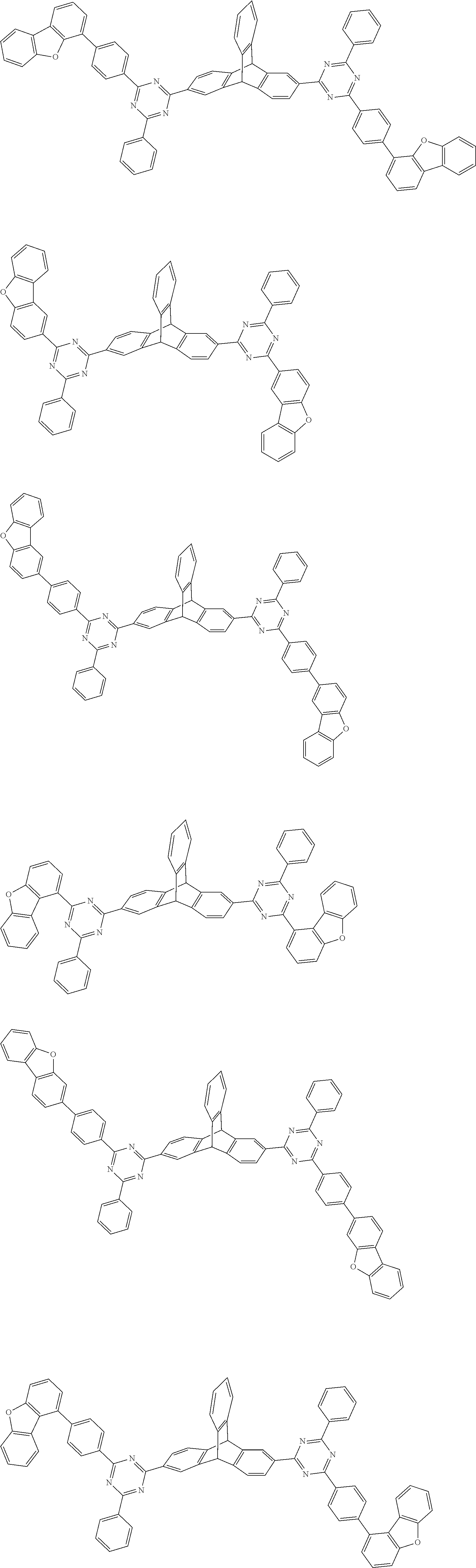

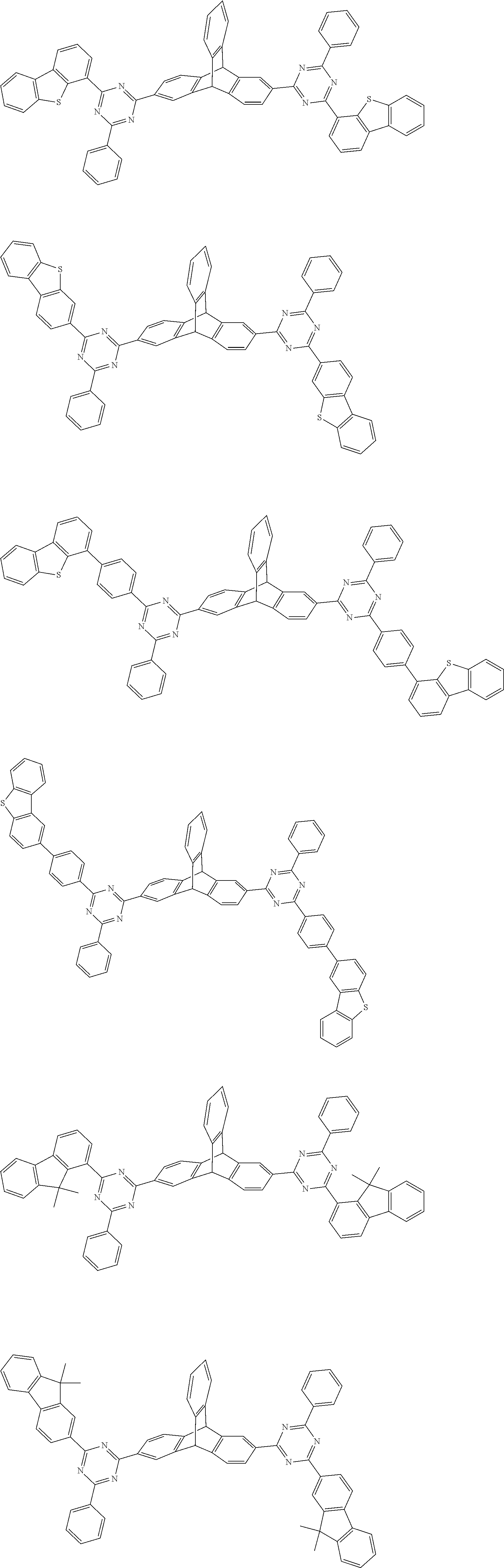

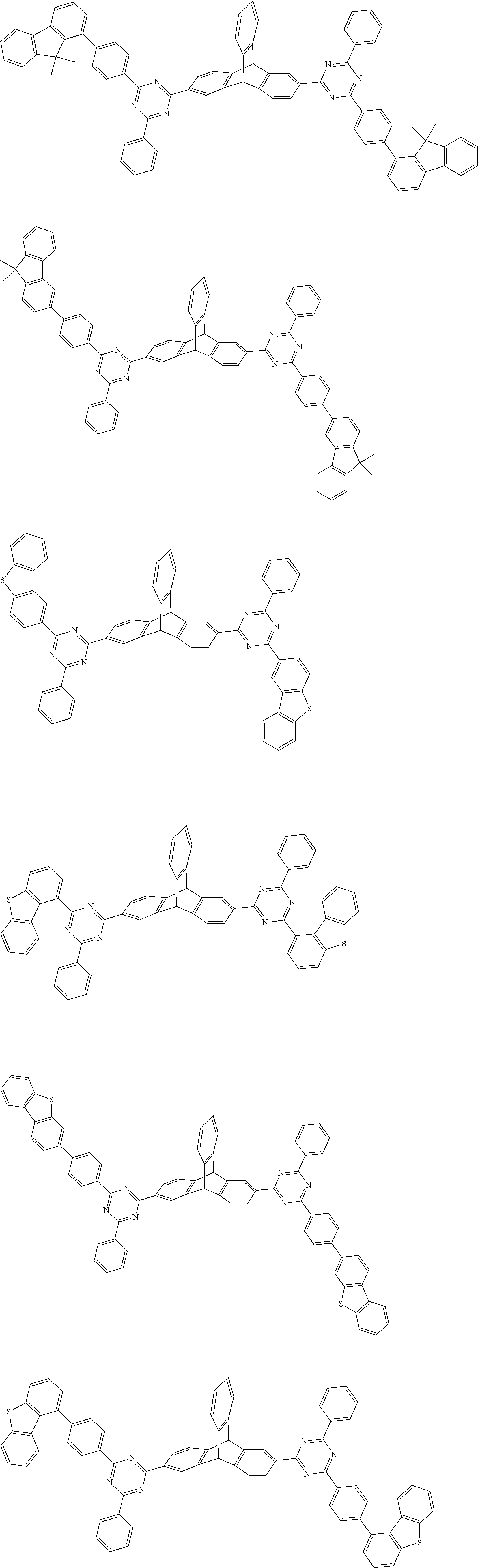

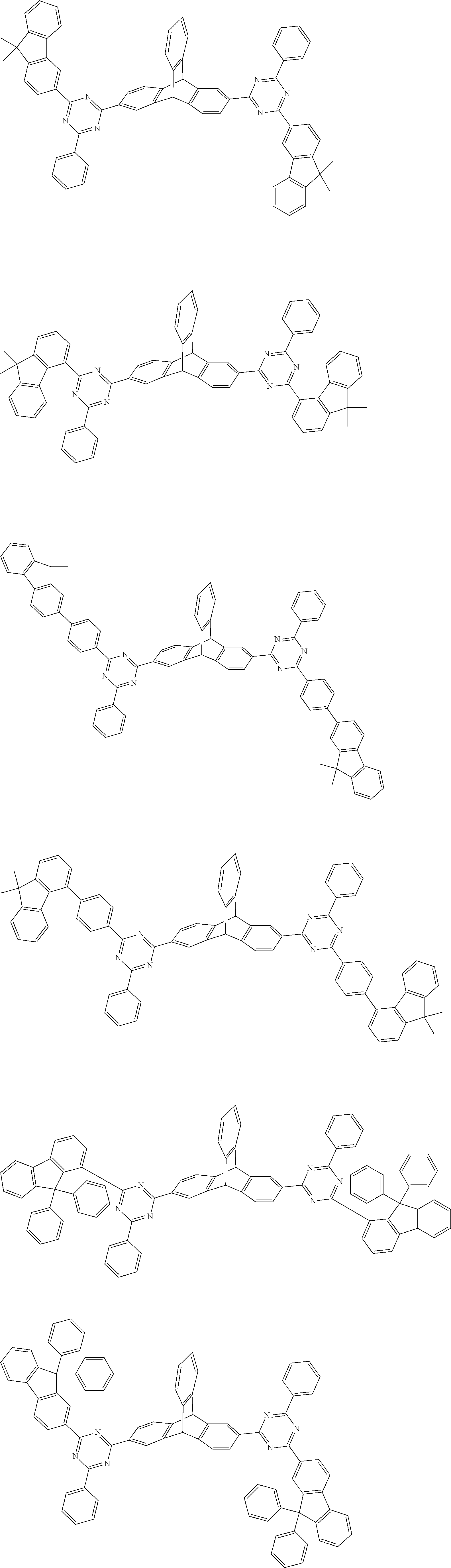

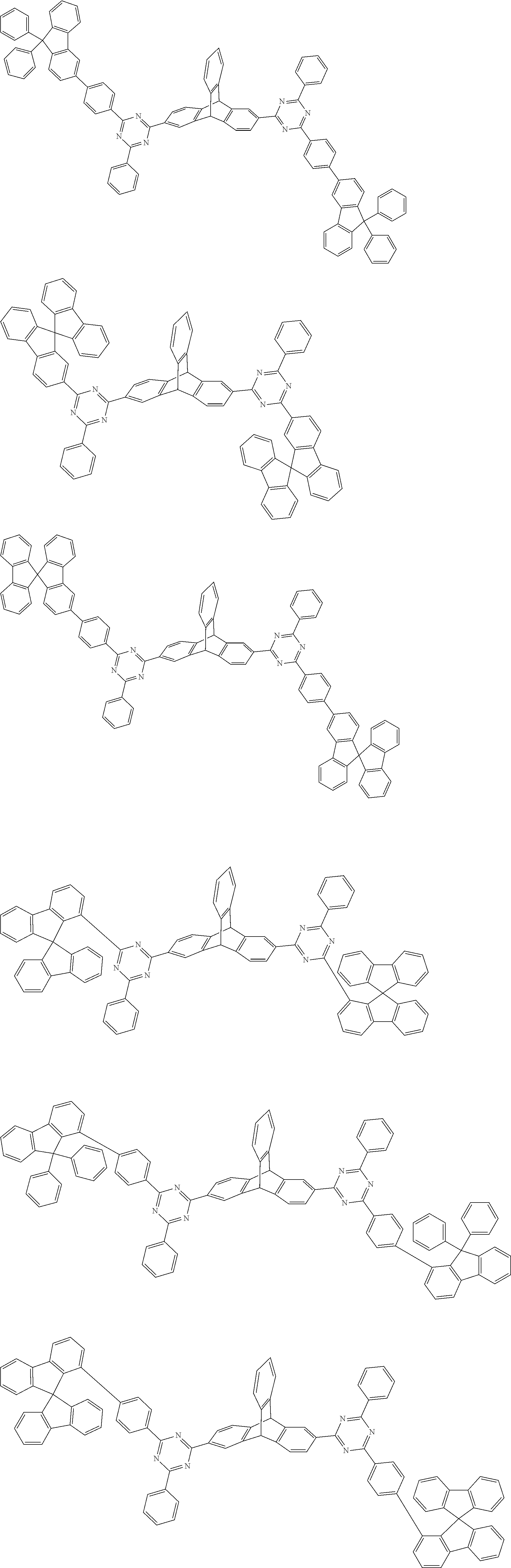

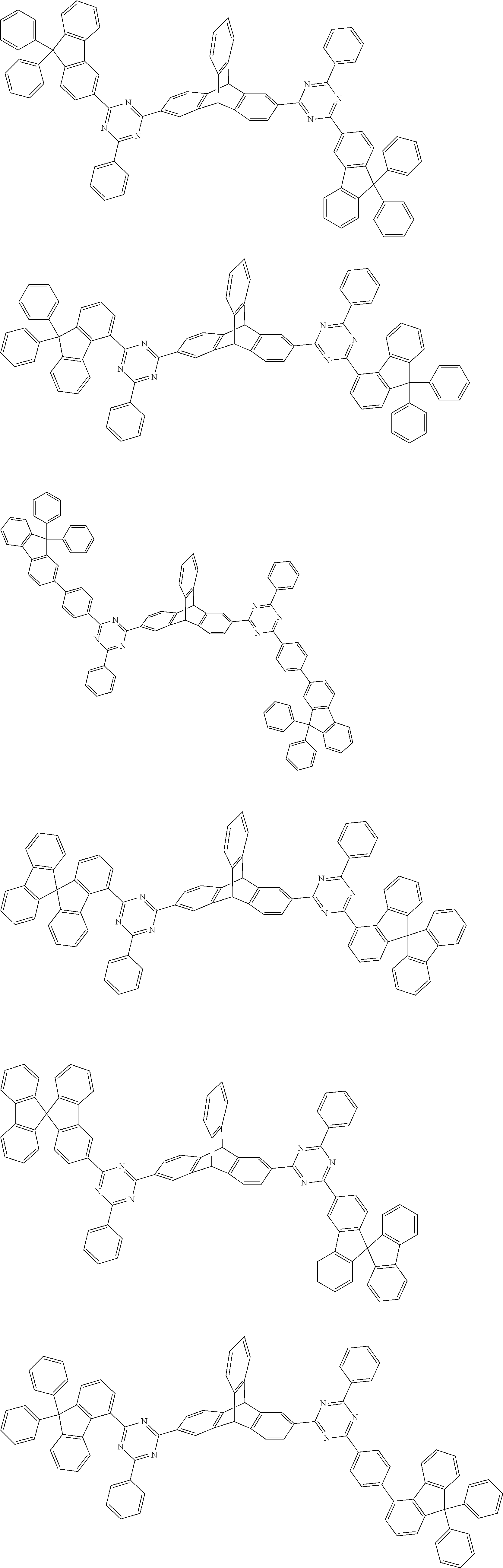

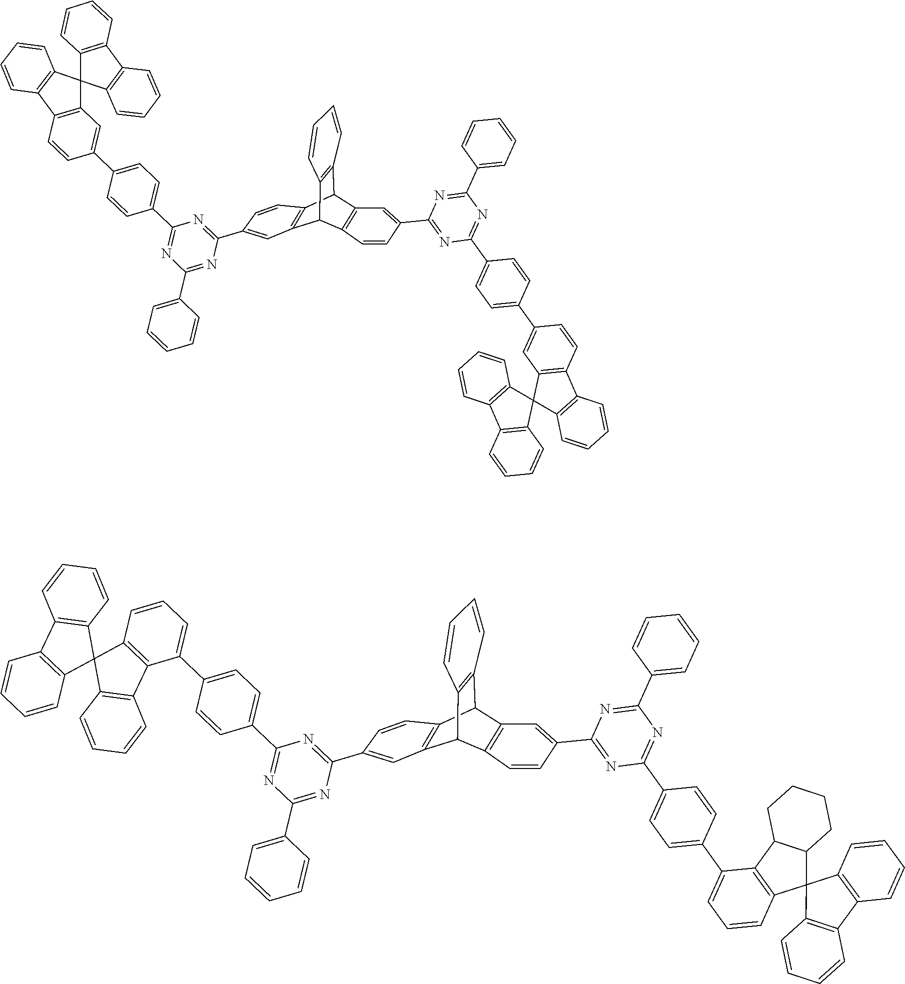 [SMILES: c1ccc(-c2nc(-c3ccc(-c4ccc5c(c4)C4(c6ccccc6-c6ccccc64)c4ccccc4-5)cc3)nc(-c3ccc4c(c3)C3c5ccccc5C4c4cc(-c5nc(-c6ccccc6)nc(-c6ccc(-c7ccc8c(c7)C7(c9ccccc9-c9ccccc97)c7ccccc7-8)cc6)n5)ccc43)n2)cc1 ; c1ccc(-c2nc(-c3ccc(-c4cccc5c4-c4ccccc4C54c5ccccc5-c5ccccc54)cc3)nc(-c3ccc4c(c3)C3c5ccccc5C4c4cc(-c5nc(-c6ccccc6)nc(-c6ccc(-c7cccc8c7C7CCCCC7C87c8ccccc8-c8ccccc87)cc6)n5)ccc43)n2)cc1]

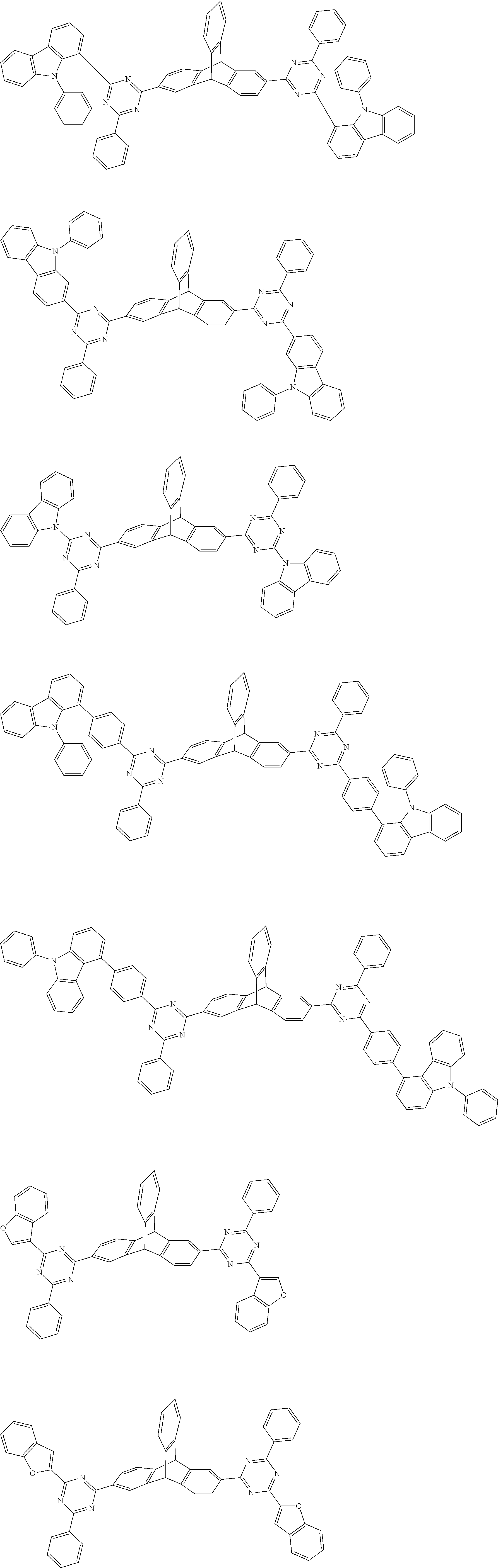

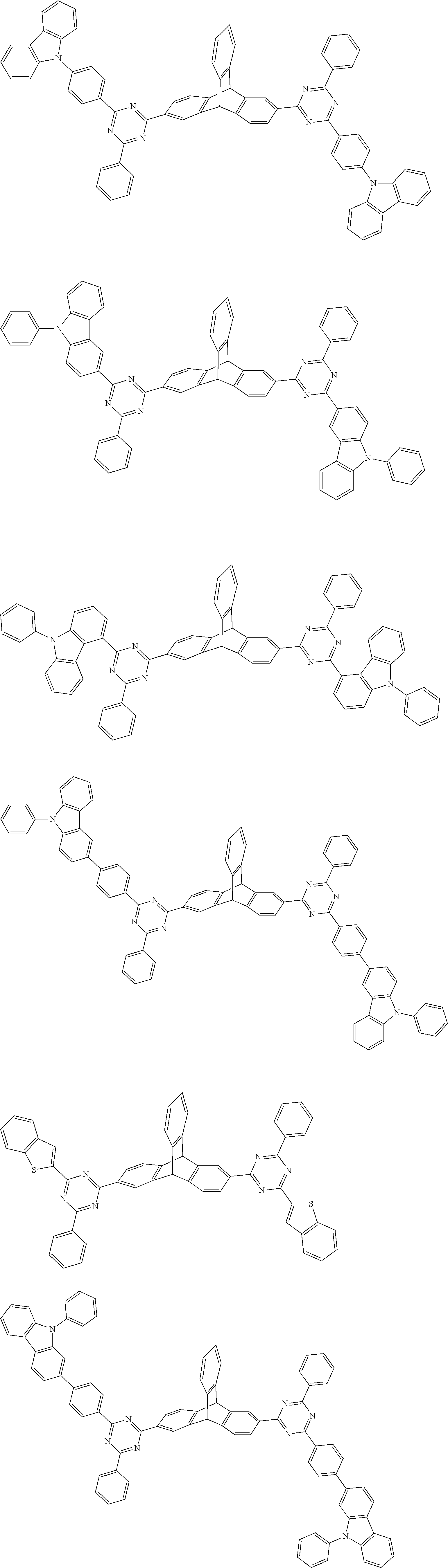

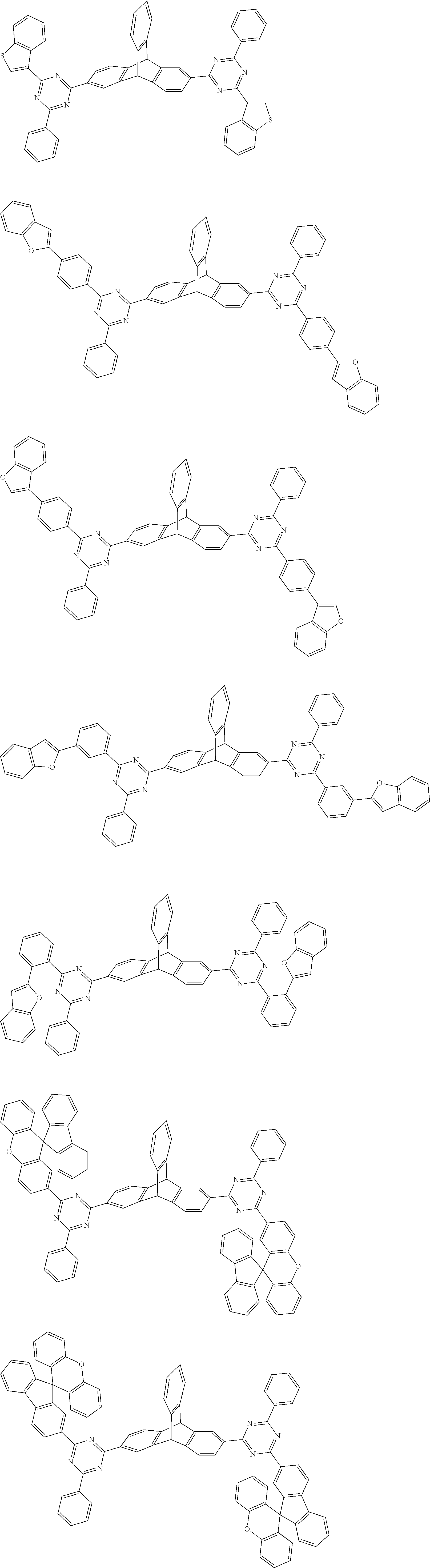

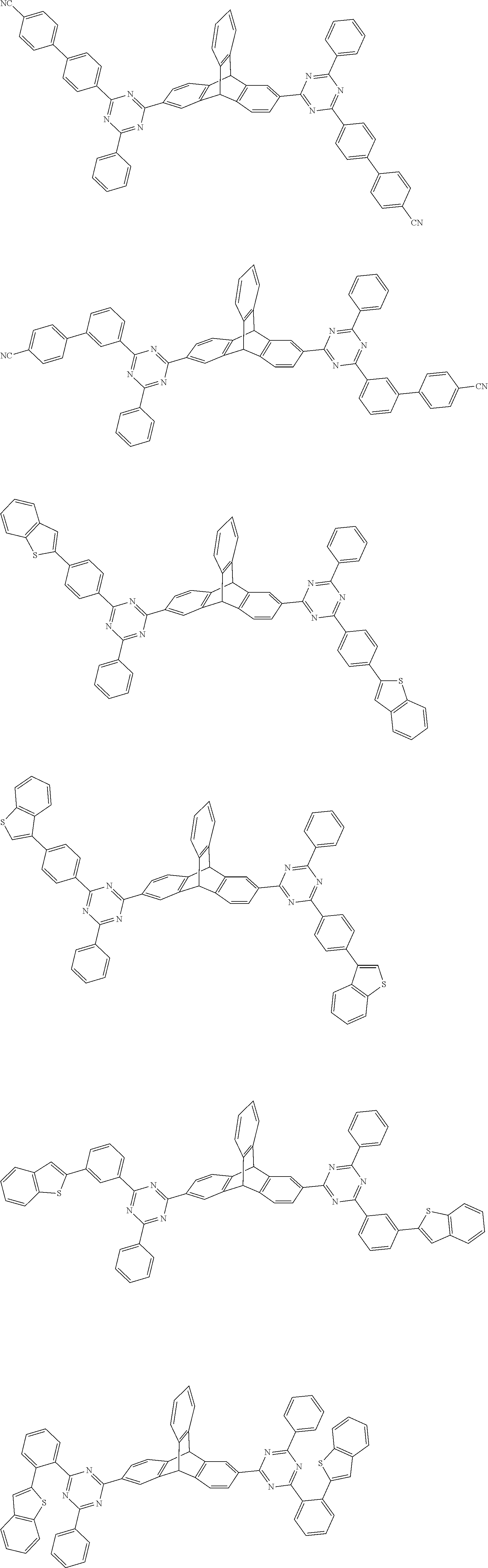

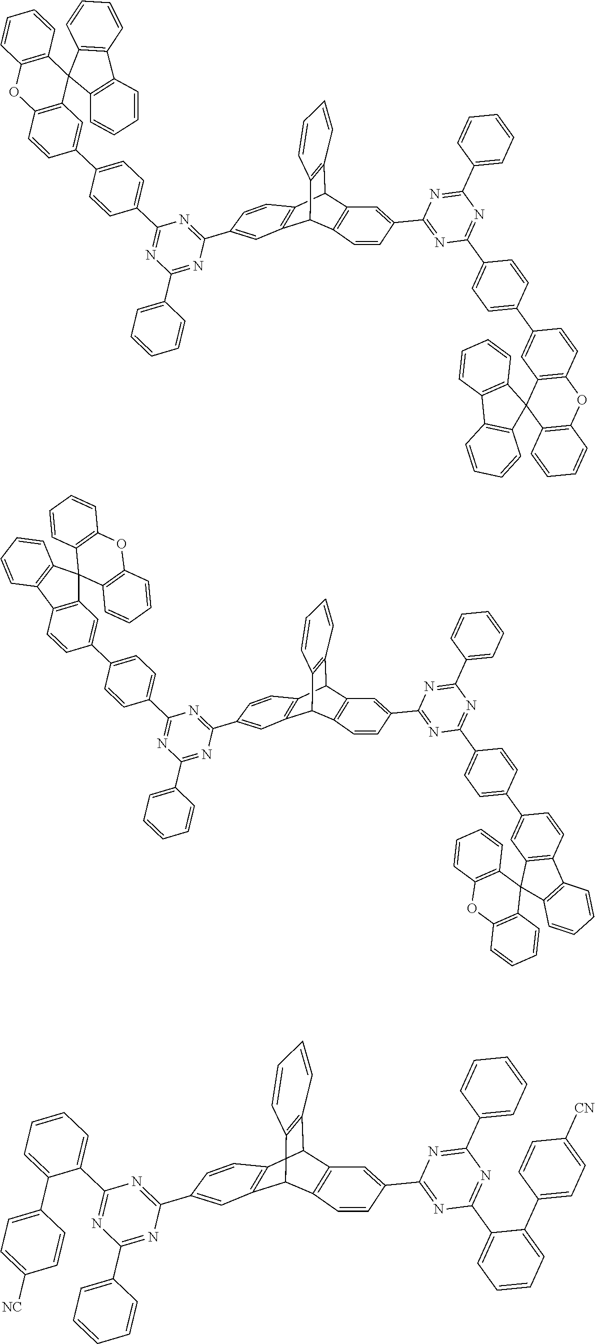

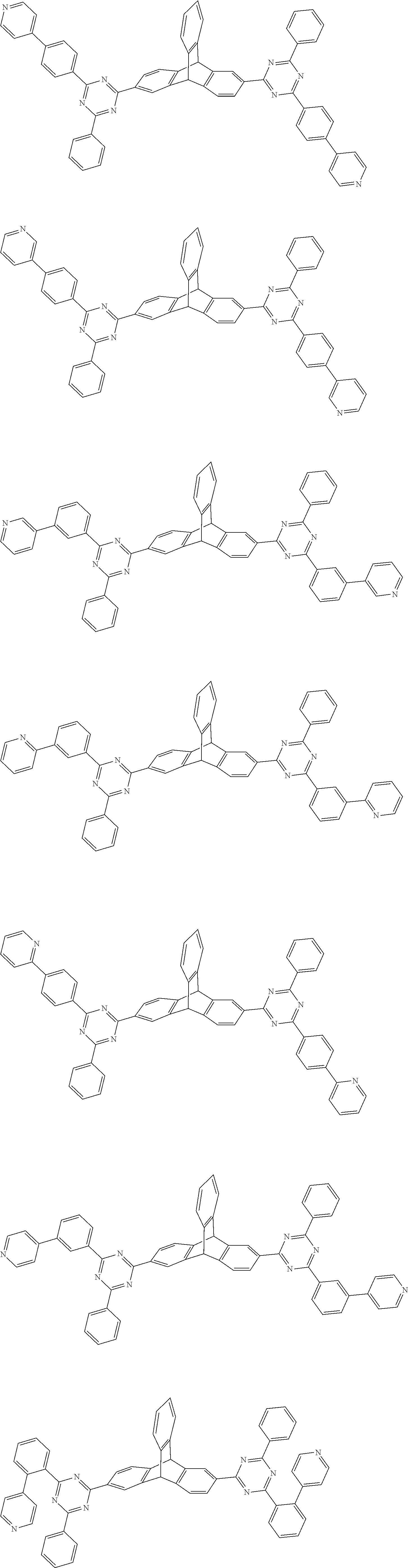

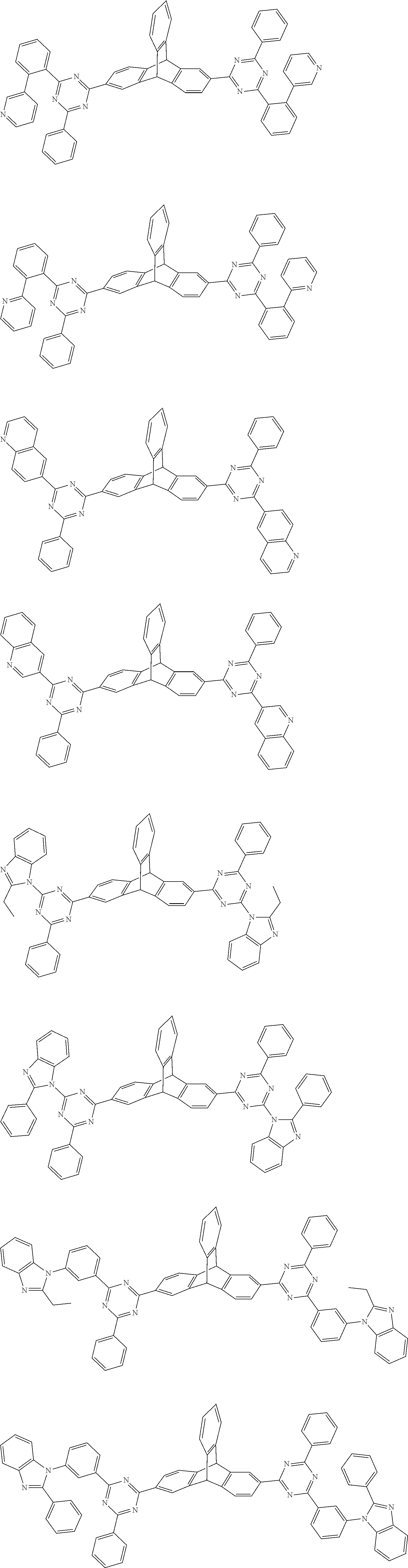

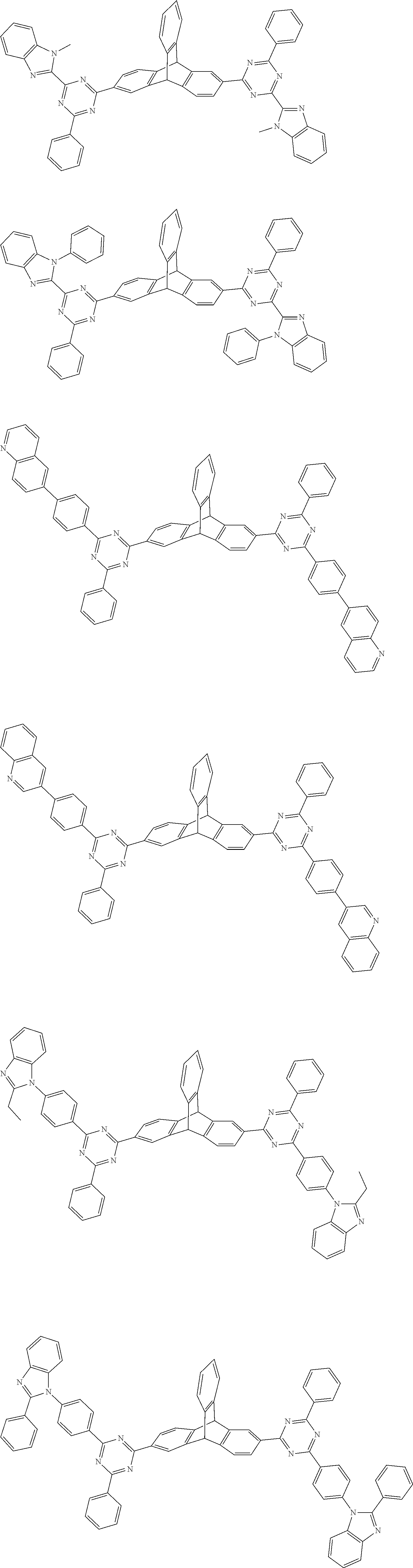

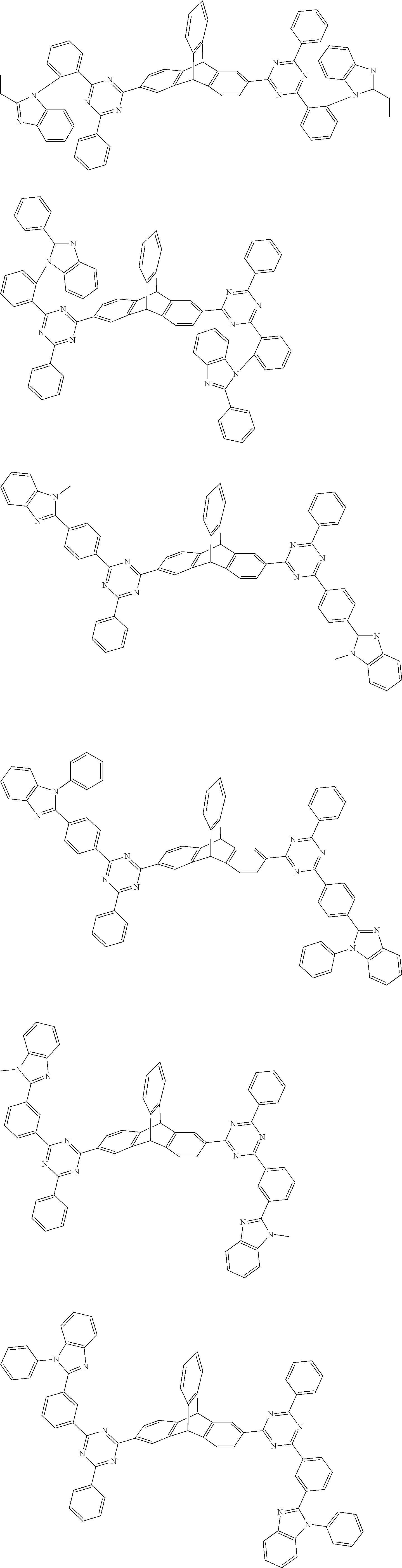

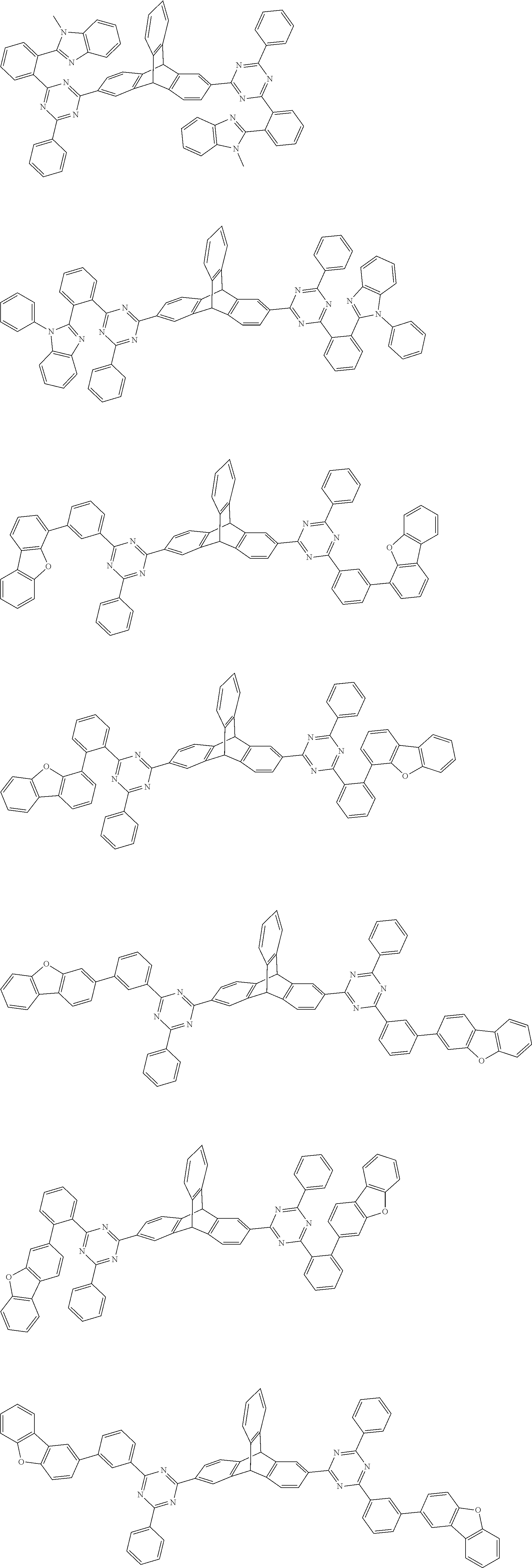

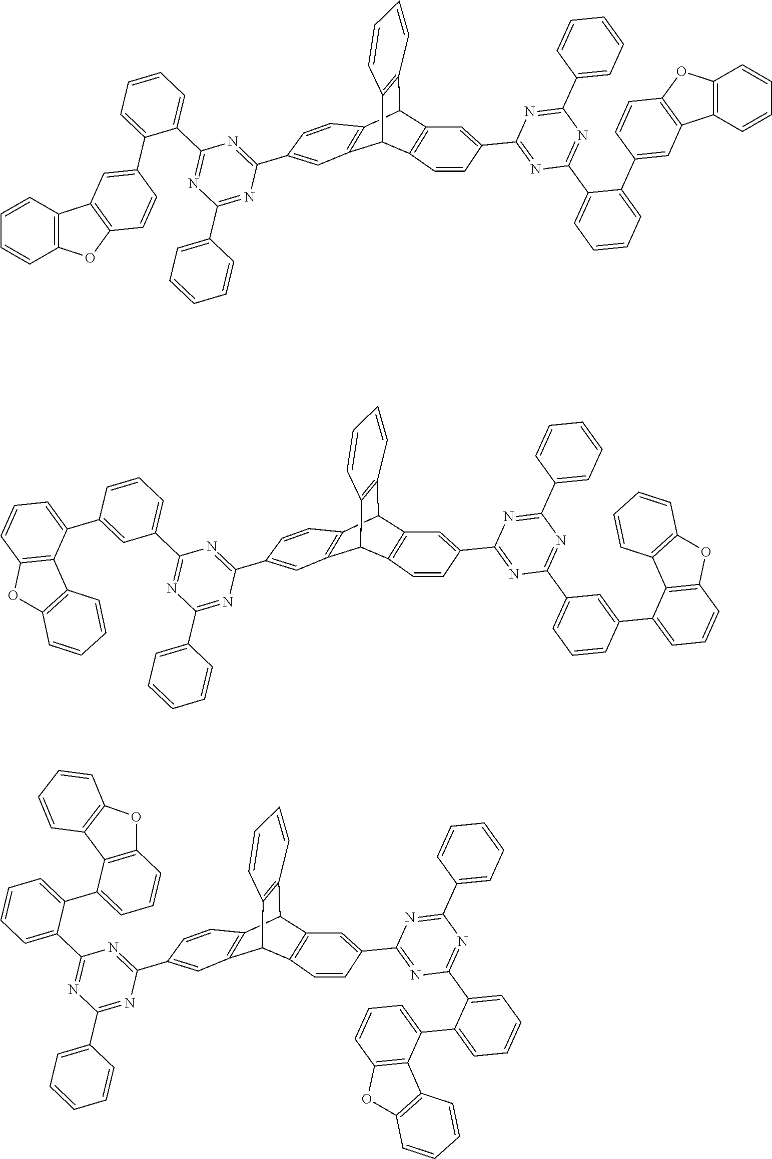 [SMILES: c1ccc(-c2nc(-c3ccc4c(c3)C3c5ccccc5C4c4cc(-c5nc(-c6ccccc6)nc(-c6ccccc6-c6ccc7oc8ccccc8c7c6)n5)ccc43)nc(-c3ccccc3-c3ccc4oc5ccccc5c4c3)n2)cc1 ; c1ccc(-c2nc(-c3cccc(-c4cccc5oc6ccccc6c45)c3)nc(-c3ccc4c(c3)C3c5ccccc5C4c4cc(-c5nc(-c6ccccc6)nc(-c6cccc(-c7cccc8oc9ccccc9c78)c6)n5)ccc43)n2)cc1 ; c1ccc(-c2nc(-c3ccc4c(c3)C3c5ccccc5C4c4cc(-c5nc(-c6ccccc6)nc(-c6ccccc6-c6cccc7oc8ccccc8c67)n5)ccc43)nc(-c3ccccc3-c3cccc4oc5ccccc5c34)n2)cc1]

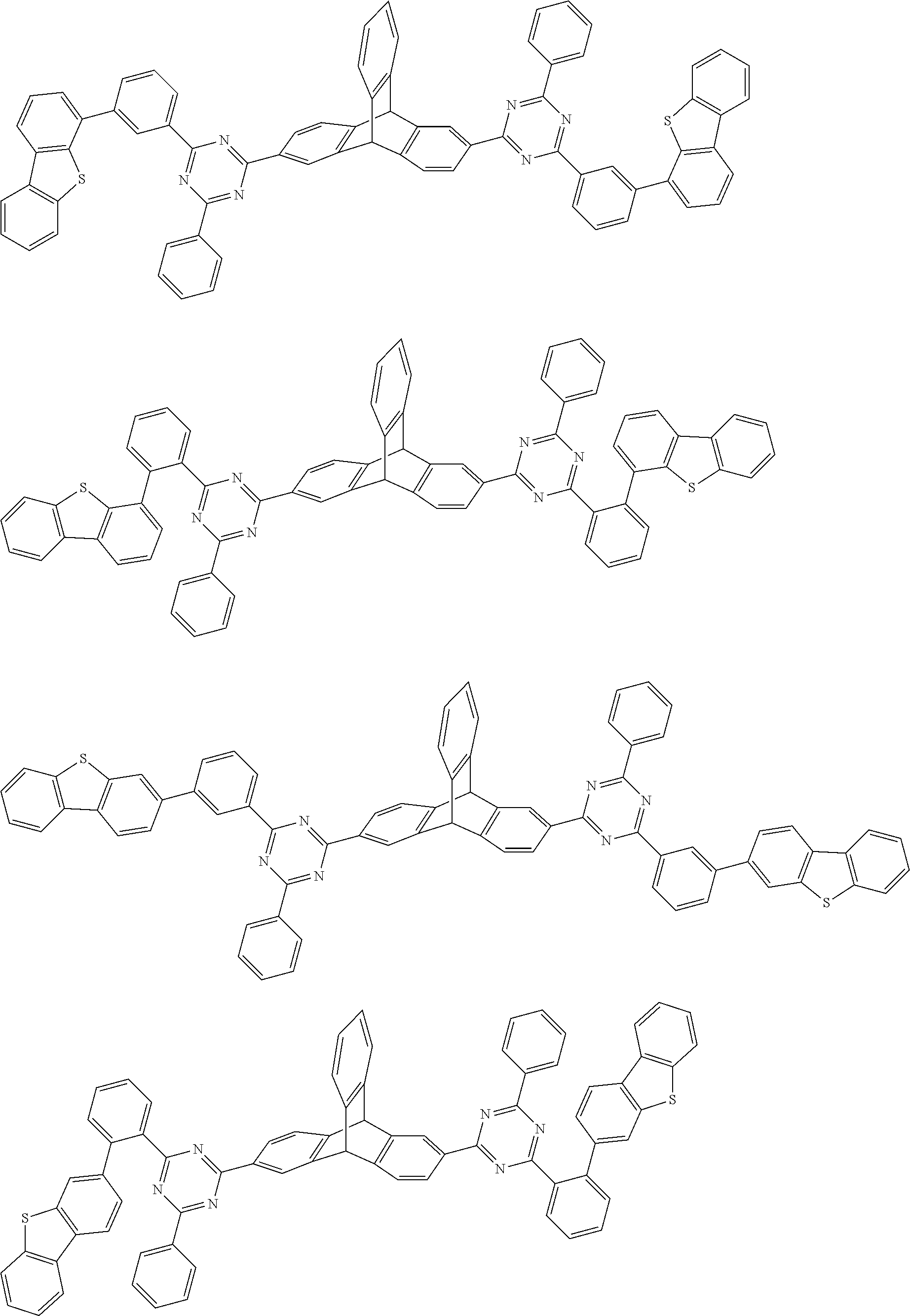

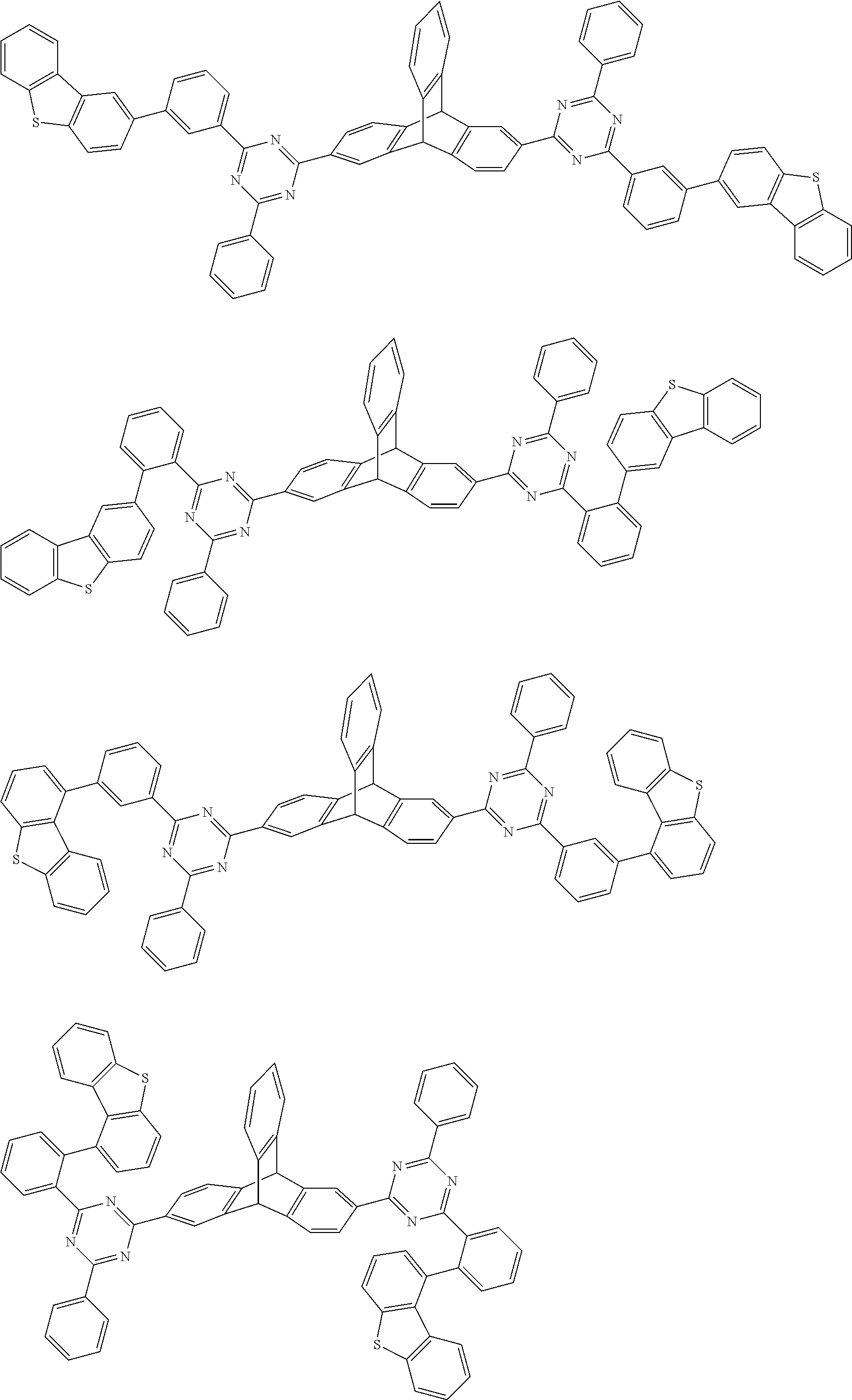

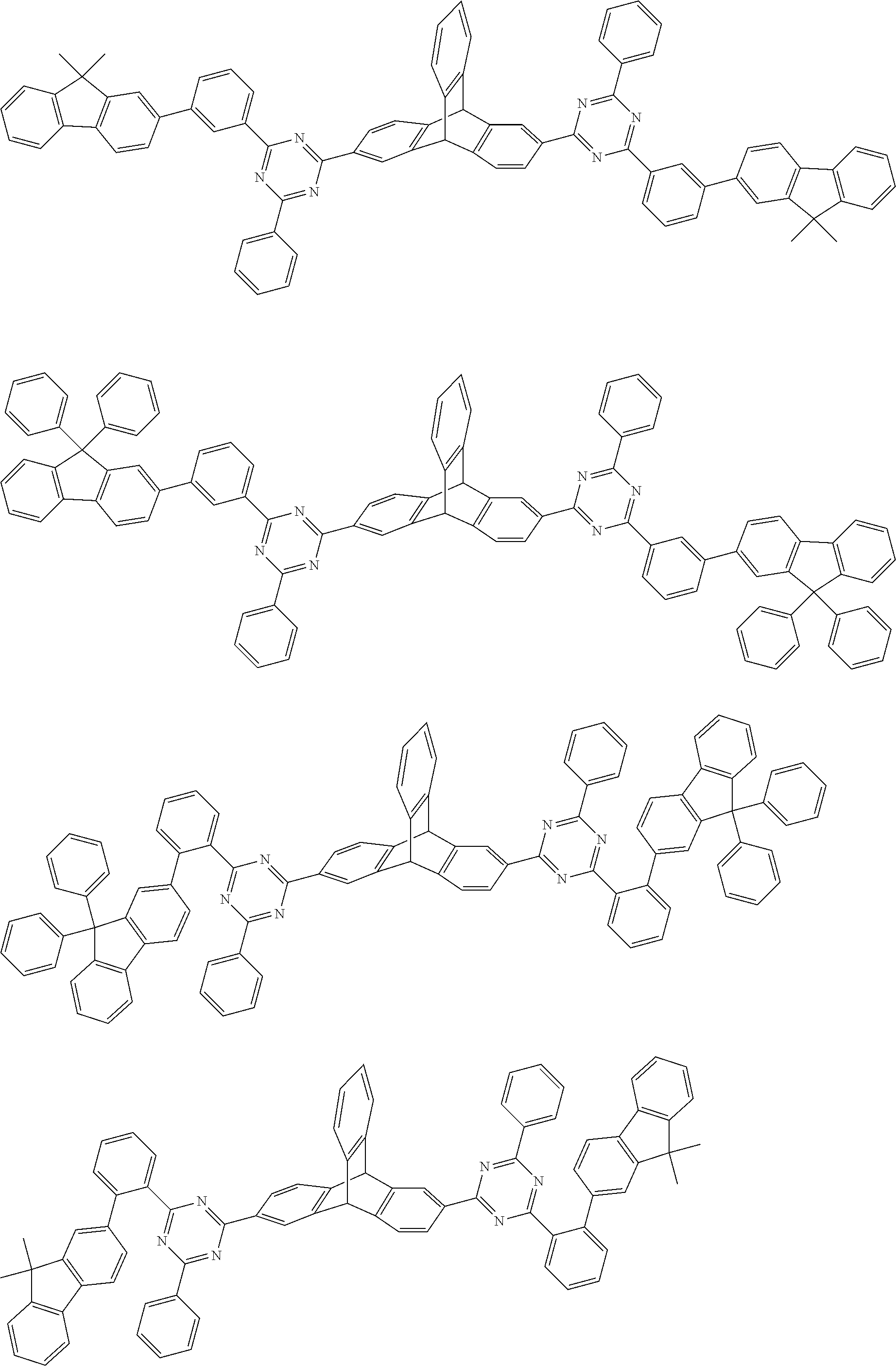

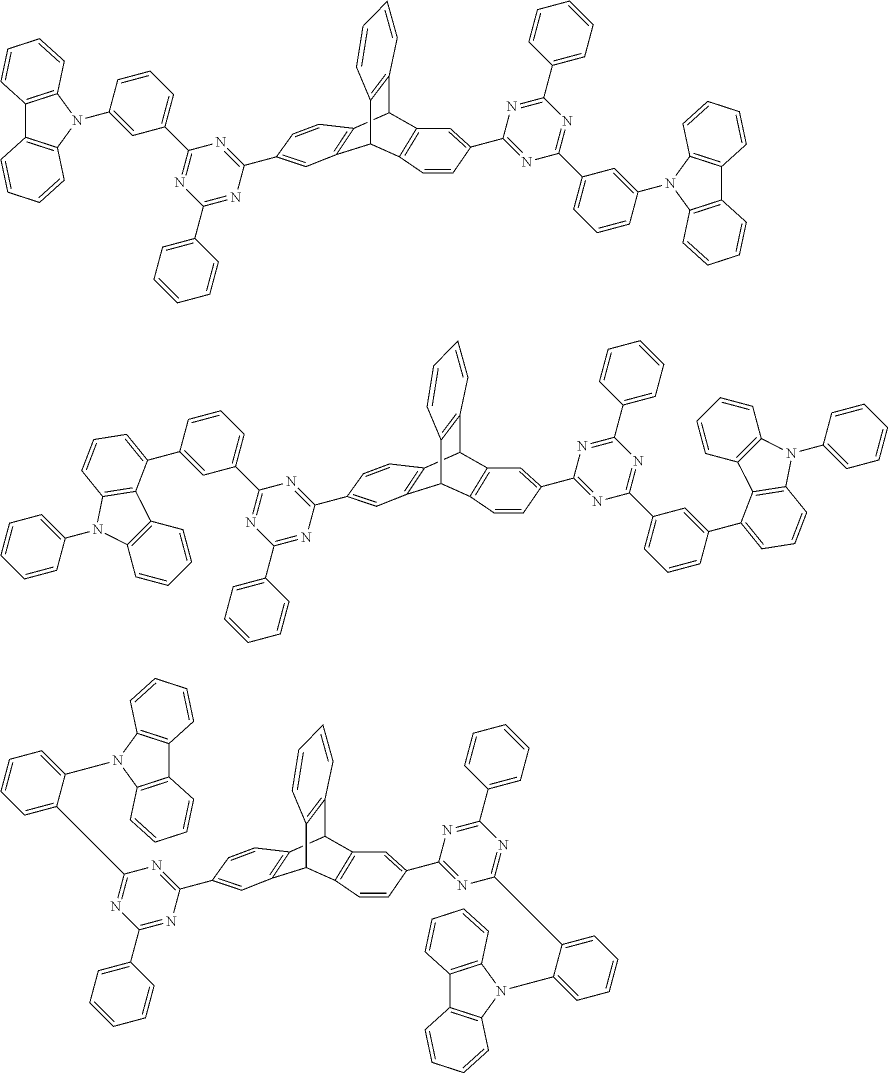 [SMILES: c1ccc(-c2nc(-c3cccc(-n4c5ccccc5c5ccccc54)c3)nc(-c3ccc4c(c3)C3c5ccccc5C4c4cc(-c5nc(-c6ccccc6)nc(-c6cccc(-n7c8ccccc8c8ccccc87)c6)n5)ccc43)n2)cc1 ; c1ccc(-c2nc(-c3cccc(-c4cccc5c4c4ccccc4n5-c4ccccc4)c3)nc(-c3ccc4c(c3)C3c5ccccc5C4c4cc(-c5nc(-c6ccccc6)nc(-c6cccc(-c7cccc8c7c7ccccc7n8-c7ccccc7)c6)n5)ccc43)n2)cc1 ; c1ccc(-c2nc(-c3ccc4c(c3)C3c5ccccc5C4c4cc(-c5nc(-c6ccccc6)nc(-c6ccccc6-n6c7ccccc7c7ccccc76)n5)ccc43)nc(-c3ccccc3-n3c4ccccc4c4ccccc43)n2)cc1]

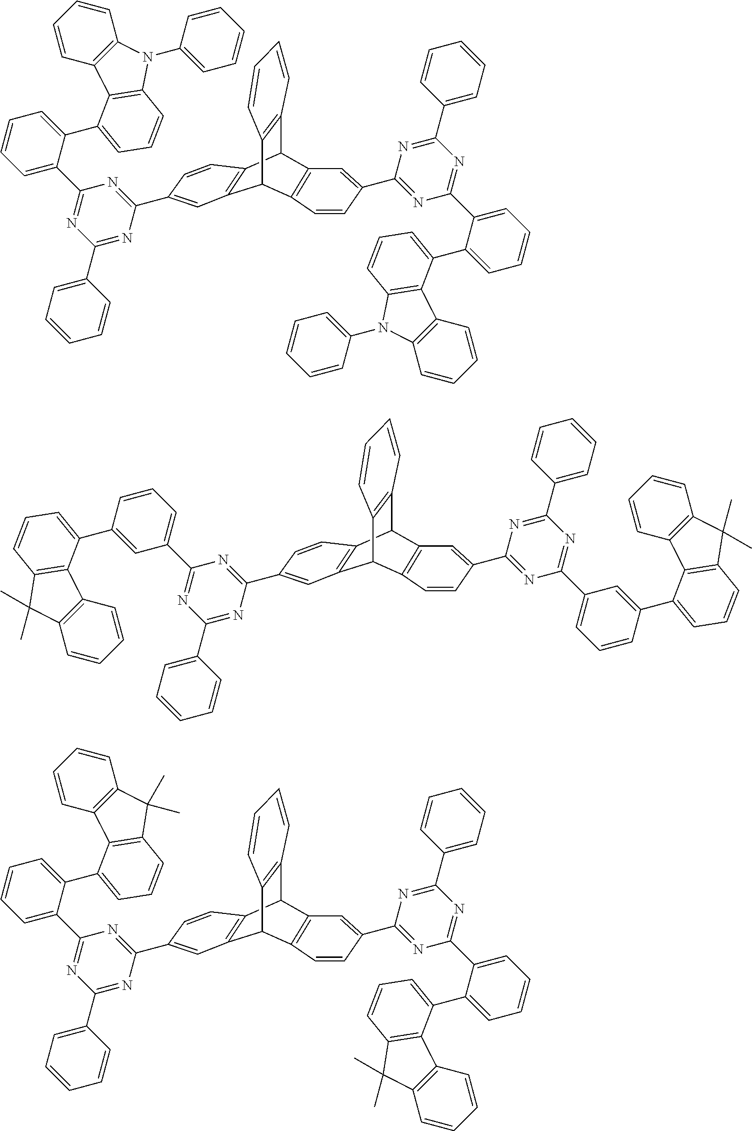

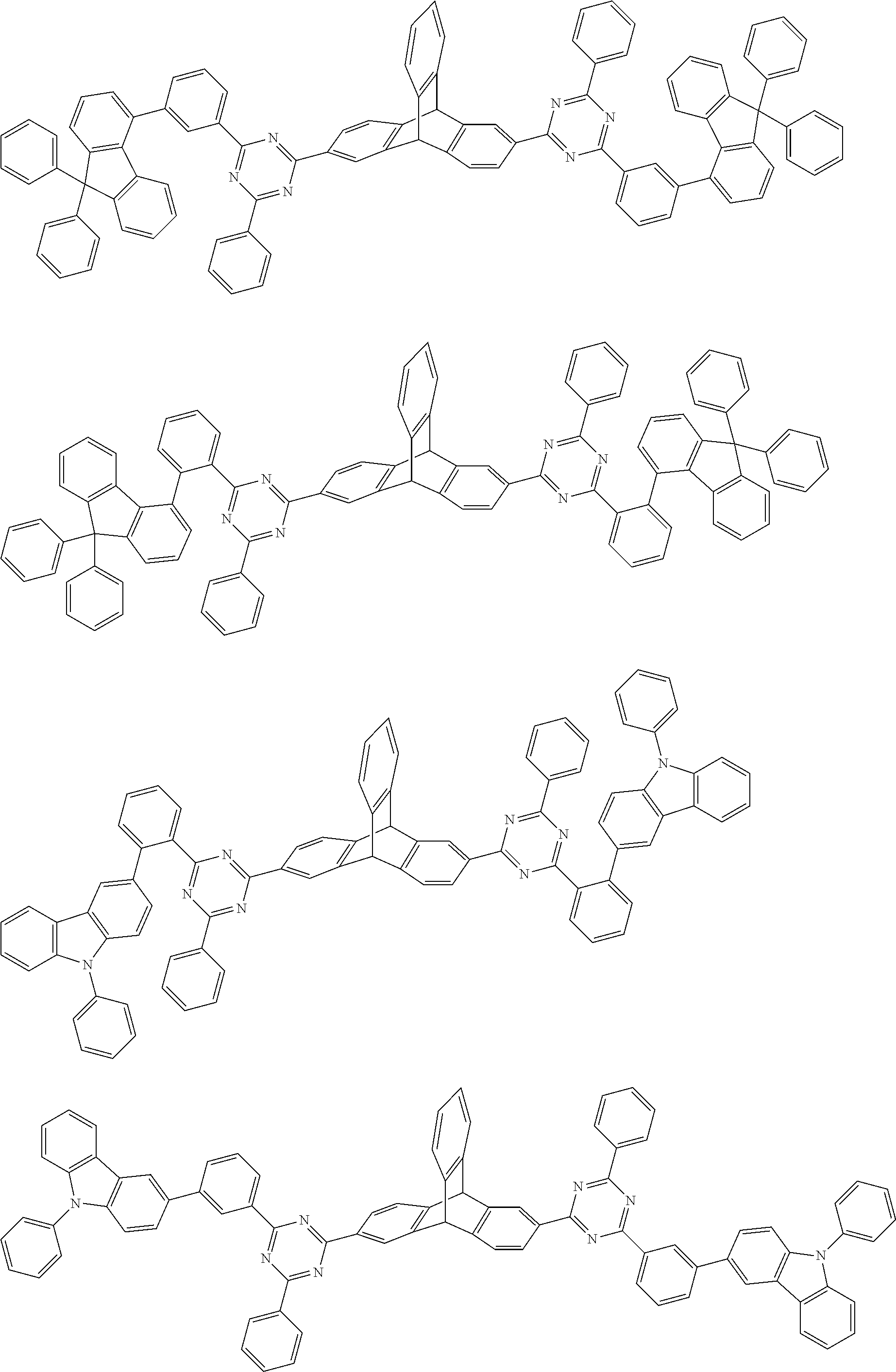

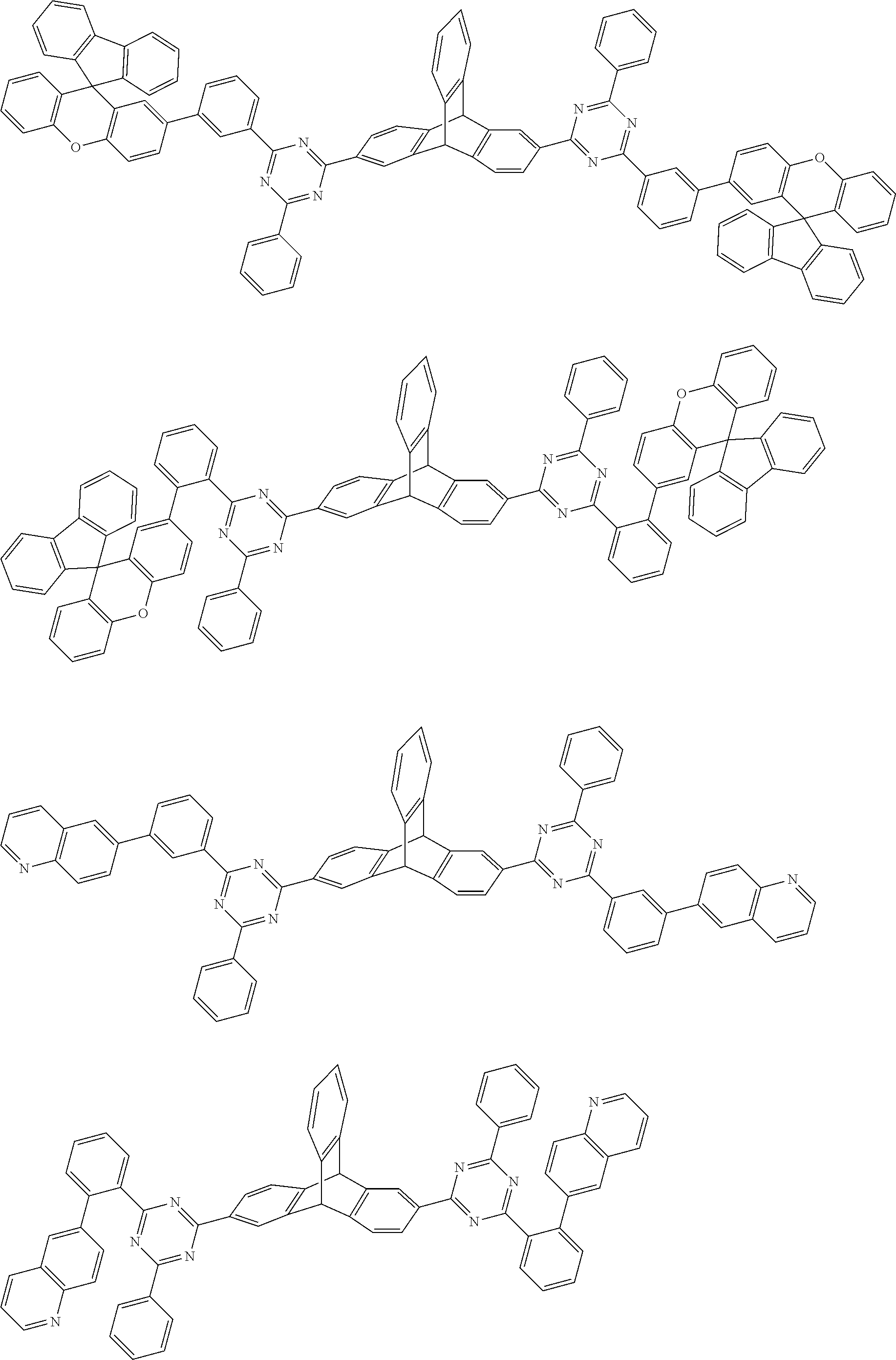

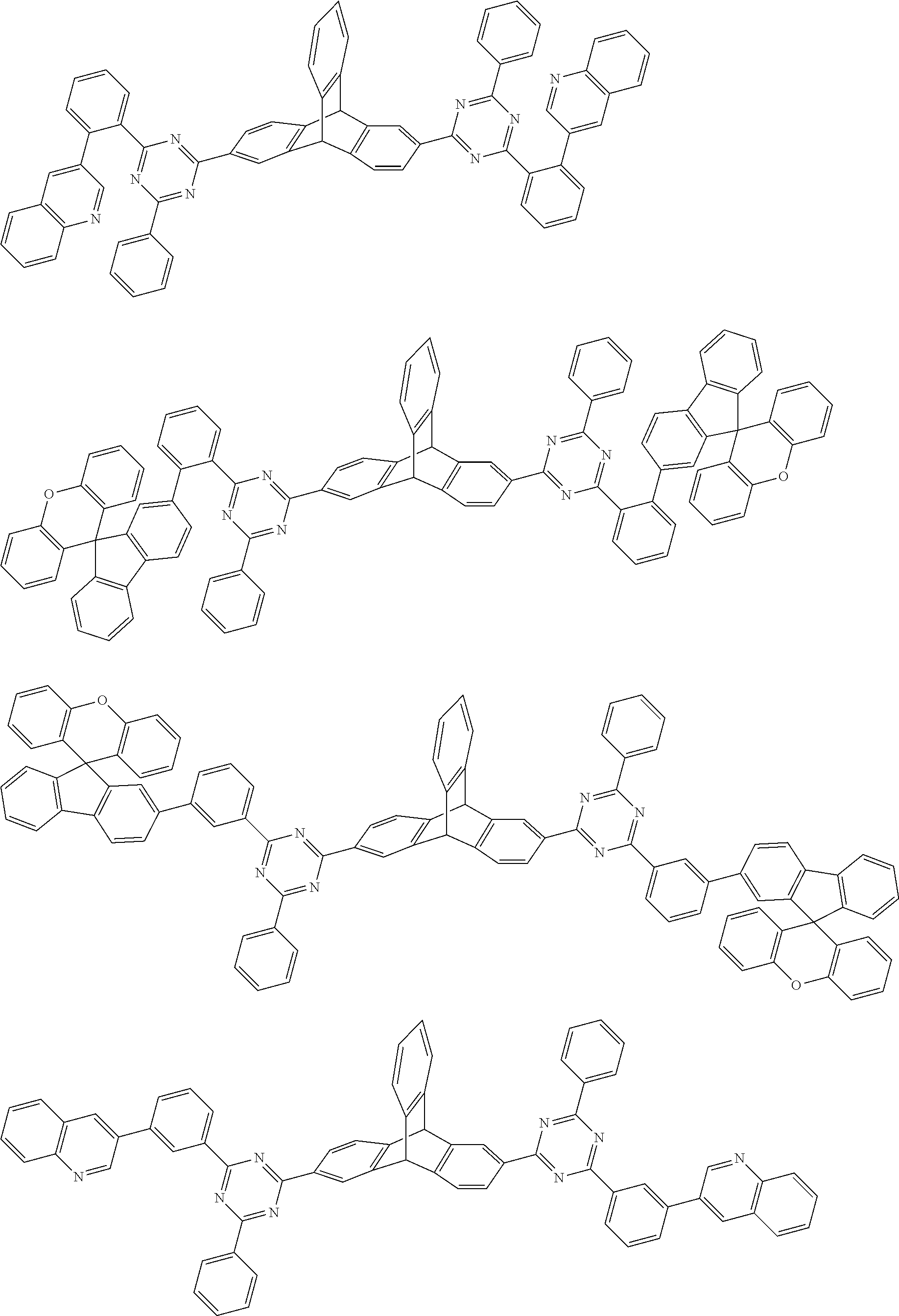

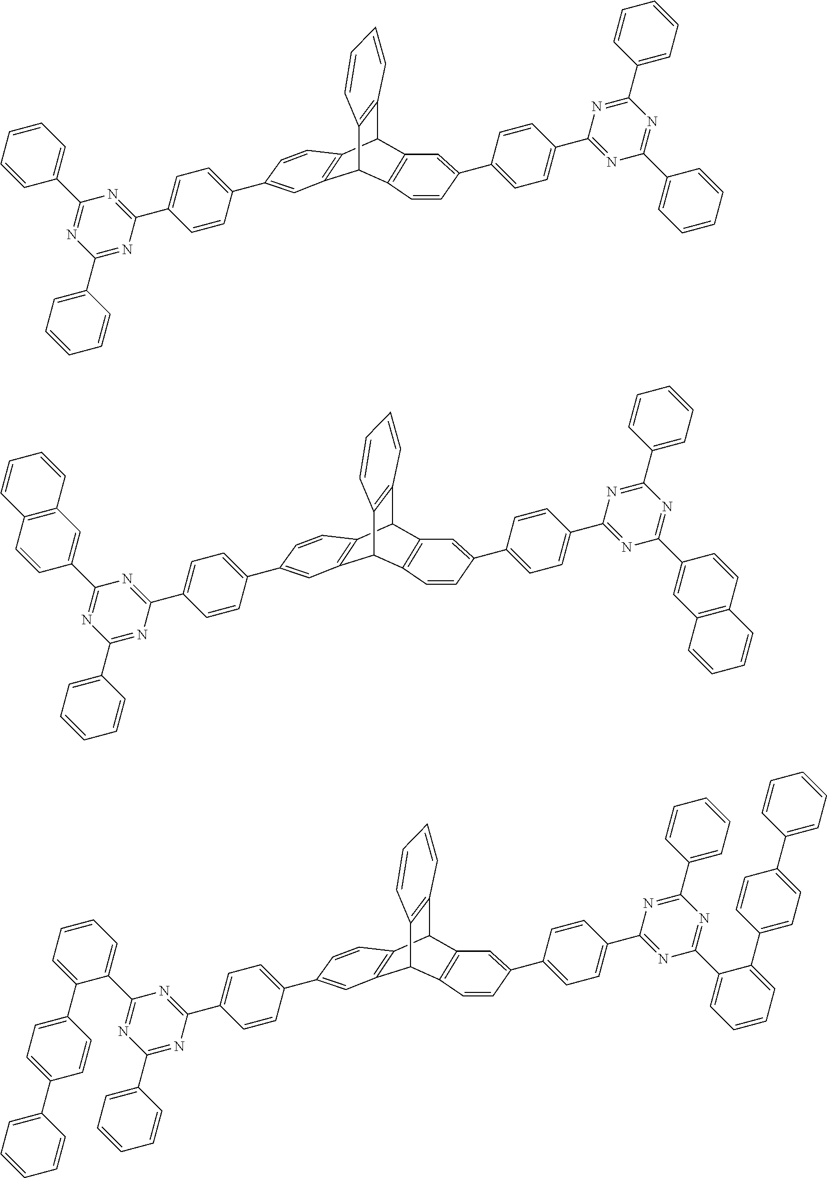 [SMILES: c1ccc(-c2nc(-c3ccccc3)nc(-c3ccc(-c4ccc5c(c4)C4c6ccccc6C5c5cc(-c6ccc(-c7nc(-c8ccccc8)nc(-c8ccccc8)n7)cc6)ccc54)cc3)n2)cc1 ; c1ccc(-c2nc(-c3ccc(-c4ccc5c(c4)C4c6ccccc6C5c5cc(-c6ccc(-c7nc(-c8ccccc8)nc(-c8ccc9ccccc9c8)n7)cc6)ccc54)cc3)nc(-c3ccc4ccccc4c3)n2)cc1 ; c1ccc(-c2ccc(-c3ccccc3-c3nc(-c4ccccc4)nc(-c4ccc(-c5ccc6c(c5)C5c7ccccc7C6c6cc(-c7ccc(-c8nc(-c9ccccc9)nc(-c9ccccc9-c9ccc(-c%10ccccc%10)cc9)n8)cc7)ccc65)cc4)n3)cc2)cc1]

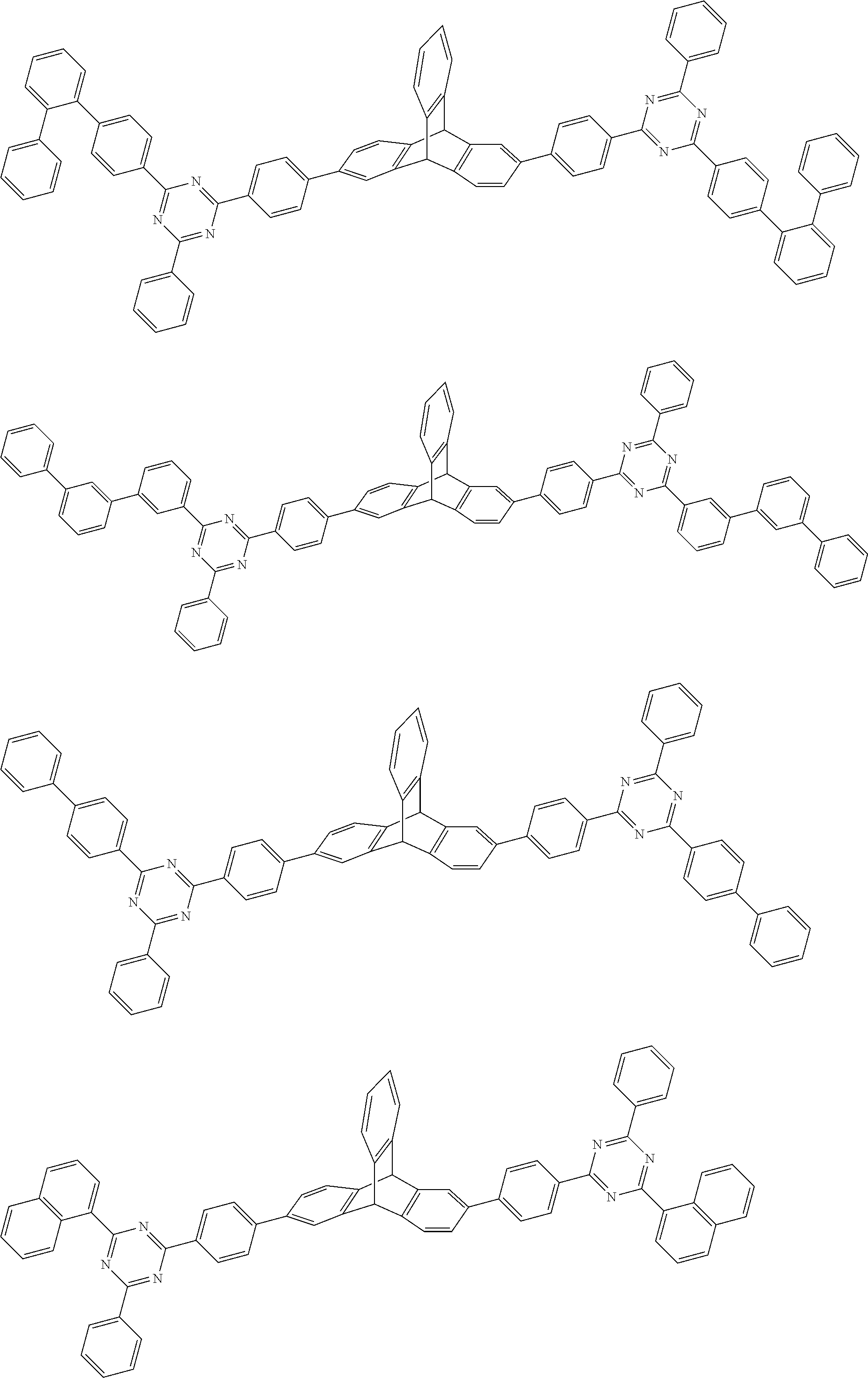

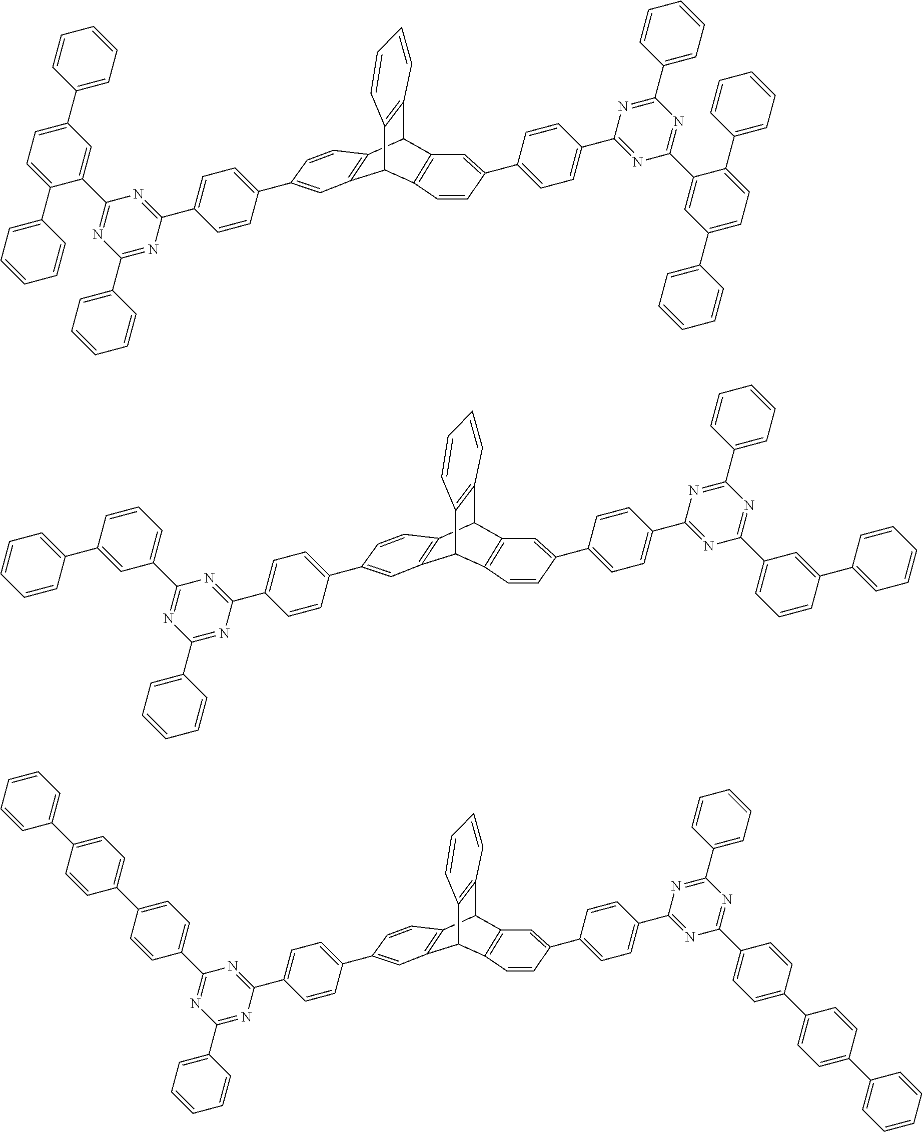 [SMILES: c1ccc(-c2ccc(-c3ccccc3)c(-c3nc(-c4ccccc4)nc(-c4ccc(-c5ccc6c(c5)C5c7ccccc7C6c6cc(-c7ccc(-c8nc(-c9ccccc9)nc(-c9cc(-c%10ccccc%10)ccc9-c9ccccc9)n8)cc7)ccc65)cc4)n3)c2)cc1 ; c1ccc(-c2cccc(-c3nc(-c4ccccc4)nc(-c4ccc(-c5ccc6c(c5)C5c7ccccc7C6c6cc(-c7ccc(-c8nc(-c9ccccc9)nc(-c9cccc(-c%10ccccc%10)c9)n8)cc7)ccc65)cc4)n3)c2)cc1 ; c1ccc(-c2ccc(-c3ccc(-c4nc(-c5ccccc5)nc(-c5ccc(-c6ccc7c(c6)C6c8ccccc8C7c7cc(-c8ccc(-c9nc(-c%10ccccc%10)nc(-c%10ccc(-c%11ccc(-c%12ccccc%12)cc%11)cc%10)n9)cc8)ccc76)cc5)n4)cc3)cc2)cc1]

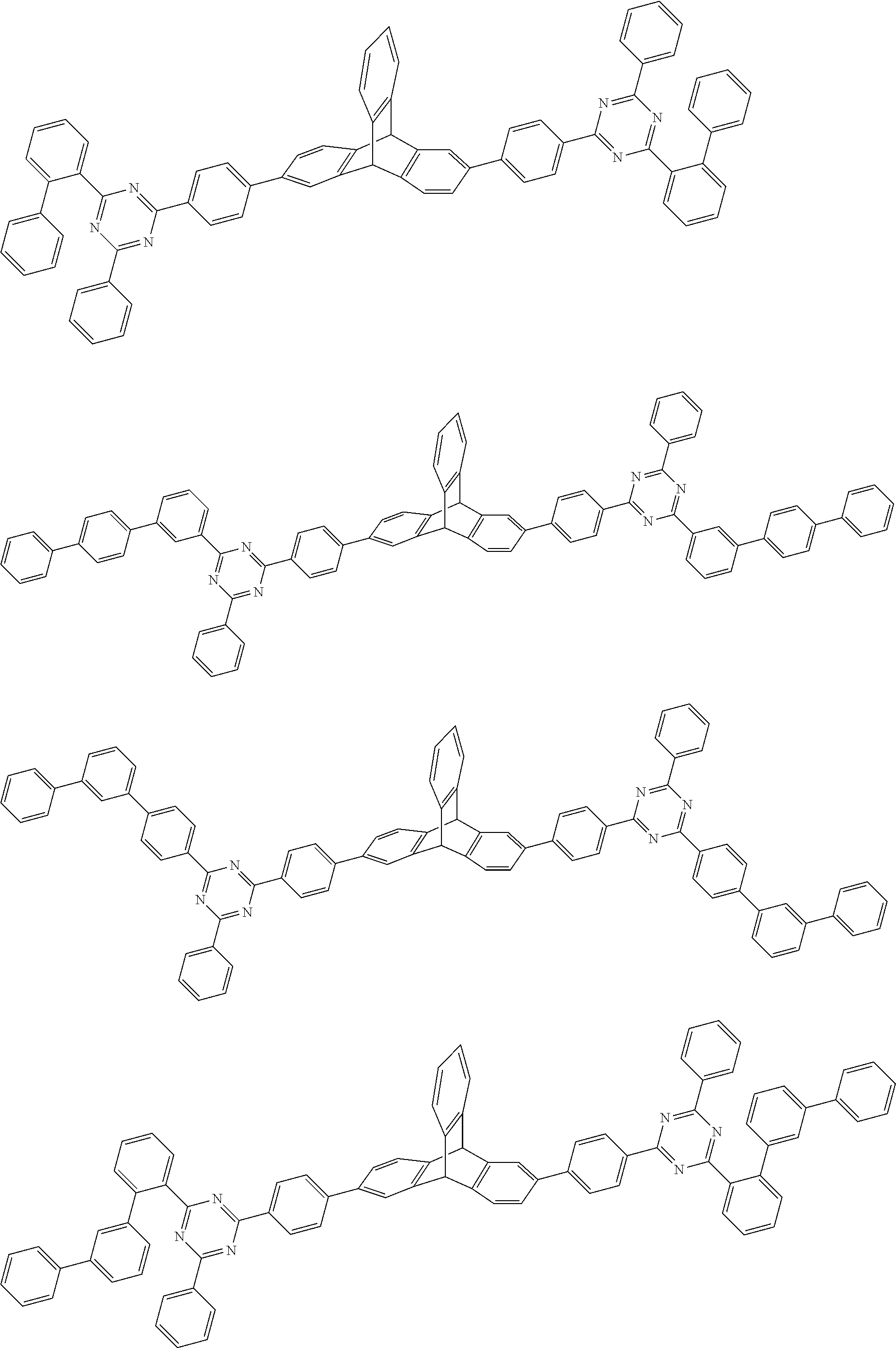

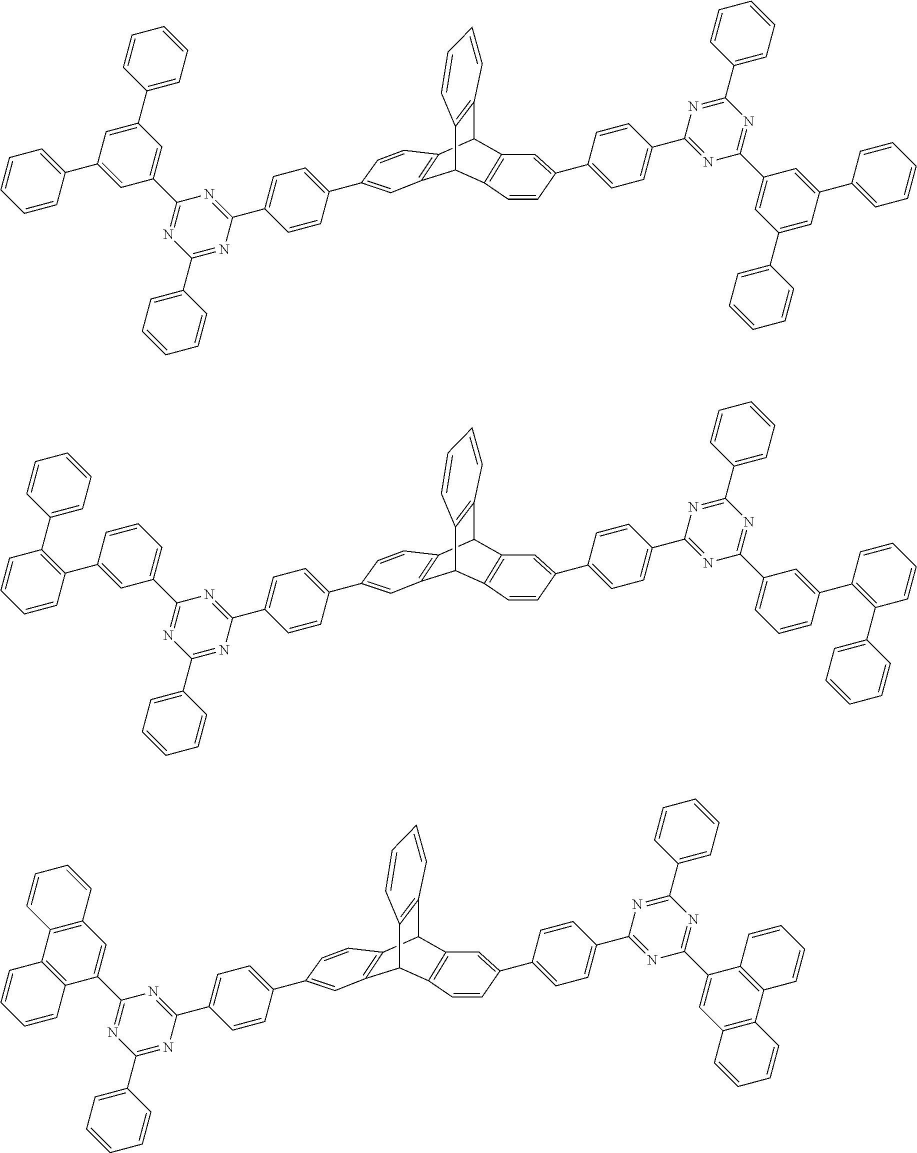 [SMILES: c1ccc(-c2cc(-c3ccccc3)cc(-c3nc(-c4ccccc4)nc(-c4ccc(-c5ccc6c(c5)C5c7ccccc7C6c6cc(-c7ccc(-c8nc(-c9ccccc9)nc(-c9cc(-c%10ccccc%10)cc(-c%10ccccc%10)c9)n8)cc7)ccc65)cc4)n3)c2)cc1 ; c1ccc(-c2nc(-c3ccc(-c4ccc5c(c4)C4c6ccccc6C5c5cc(-c6ccc(-c7nc(-c8ccccc8)nc(-c8cccc(-c9ccccc9-c9ccccc9)c8)n7)cc6)ccc54)cc3)nc(-c3cccc(-c4ccccc4-c4ccccc4)c3)n2)cc1 ; c1ccc(-c2nc(-c3ccc(-c4ccc5c(c4)C4c6ccccc6C5c5cc(-c6ccc(-c7nc(-c8ccccc8)nc(-c8cc9ccccc9c9ccccc89)n7)cc6)ccc54)cc3)nc(-c3cc4ccccc4c4ccccc34)n2)cc1]

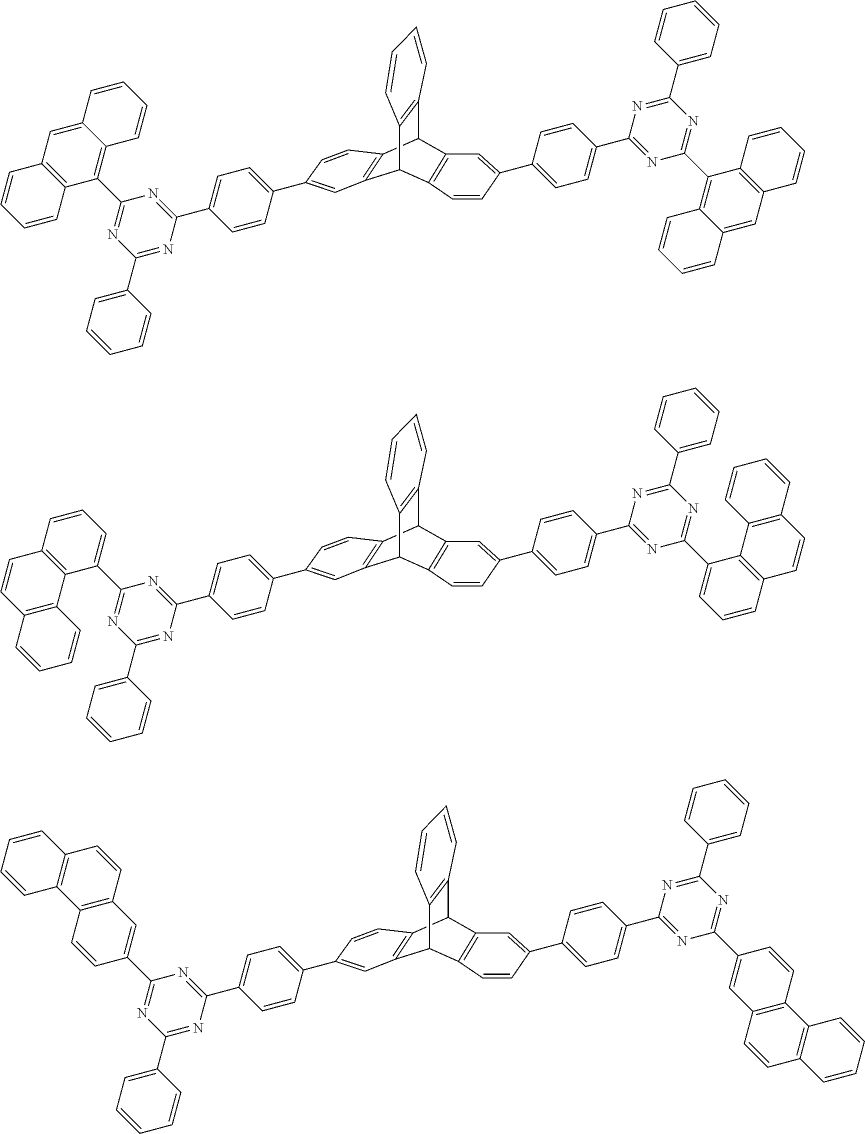 [SMILES: c1ccc(-c2nc(-c3ccc(-c4ccc5c(c4)C4c6ccccc6C5c5cc(-c6ccc(-c7nc(-c8ccccc8)nc(-c8c9ccccc9cc9ccccc89)n7)cc6)ccc54)cc3)nc(-c3c4ccccc4cc4ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8cccc9ccc%10ccccc%10c89)n7)cc6)ccc54)cc3)nc(-c3cccc4ccc5ccccc5c34)n2)cc1 ; c1ccc(-c2nc(-c3ccc(-c4ccc5c(c4)C4c6ccccc6C5c5cc(-c6ccc(-c7nc(-c8ccccc8)nc(-c8ccc9c(ccc%10ccccc%109)c8)n7)cc6)ccc54)cc3)nc(-c3ccc4c(ccc5ccccc54)c3)n2)cc1]

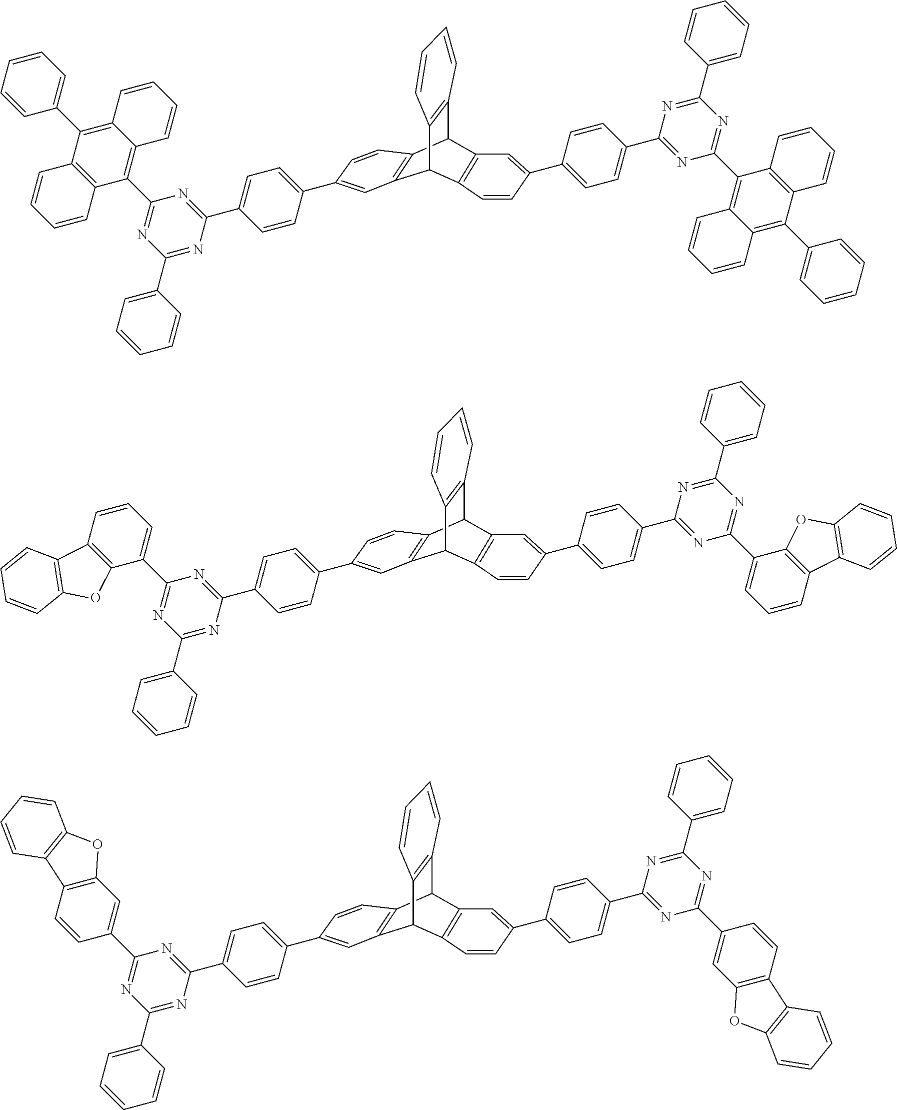 [SMILES: c1ccc(-c2nc(-c3ccc(-c4ccc5c(c4)C4c6ccccc6C5c5cc(-c6ccc(-c7nc(-c8ccccc8)nc(-c8c9ccccc9c(-c9ccccc9)c9ccccc89)n7)cc6)ccc54)cc3)nc(-c3c4ccccc4c(-c4ccccc4)c4ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8cccc9c8oc8ccccc89)n7)cc6)ccc54)cc3)nc(-c3cccc4c3oc3ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8ccc9c(c8)oc8ccccc89)n7)cc6)ccc54)cc3)nc(-c3ccc4c(c3)oc3ccccc34)n2)cc1]

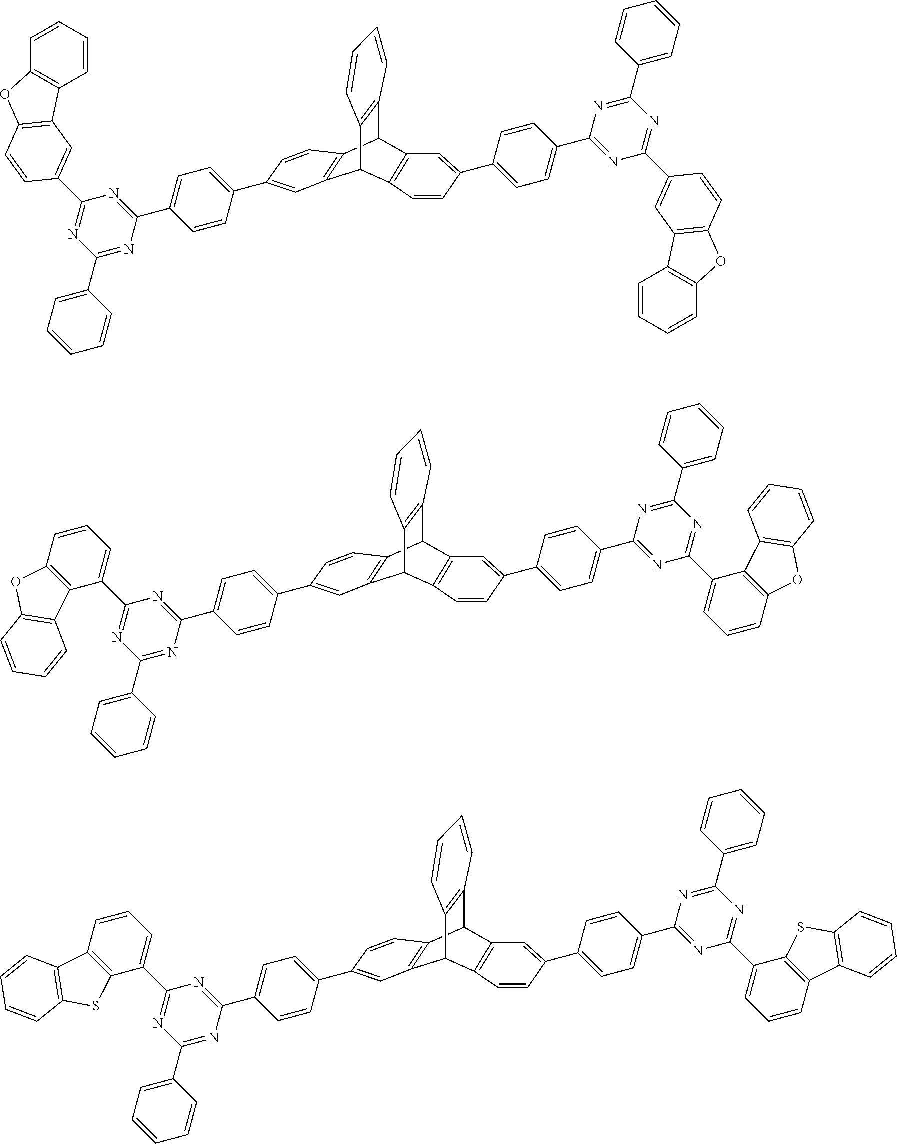 [SMILES: c1ccc(-c2nc(-c3ccc(-c4ccc5c(c4)C4c6ccccc6C5c5cc(-c6ccc(-c7nc(-c8ccccc8)nc(-c8ccc9oc%10ccccc%10c9c8)n7)cc6)ccc54)cc3)nc(-c3ccc4oc5ccccc5c4c3)n2)cc1 ; c1ccc(-c2nc(-c3ccc(-c4ccc5c(c4)C4c6ccccc6C5c5cc(-c6ccc(-c7nc(-c8ccccc8)nc(-c8cccc9oc%10ccccc%10c89)n7)cc6)ccc54)cc3)nc(-c3cccc4oc5ccccc5c34)n2)cc1 ; c1ccc(-c2nc(-c3ccc(-c4ccc5c(c4)C4c6ccccc6C5c5cc(-c6ccc(-c7nc(-c8ccccc8)nc(-c8cccc9c8sc8ccccc89)n7)cc6)ccc54)cc3)nc(-c3cccc4c3sc3ccccc34)n2)cc1]

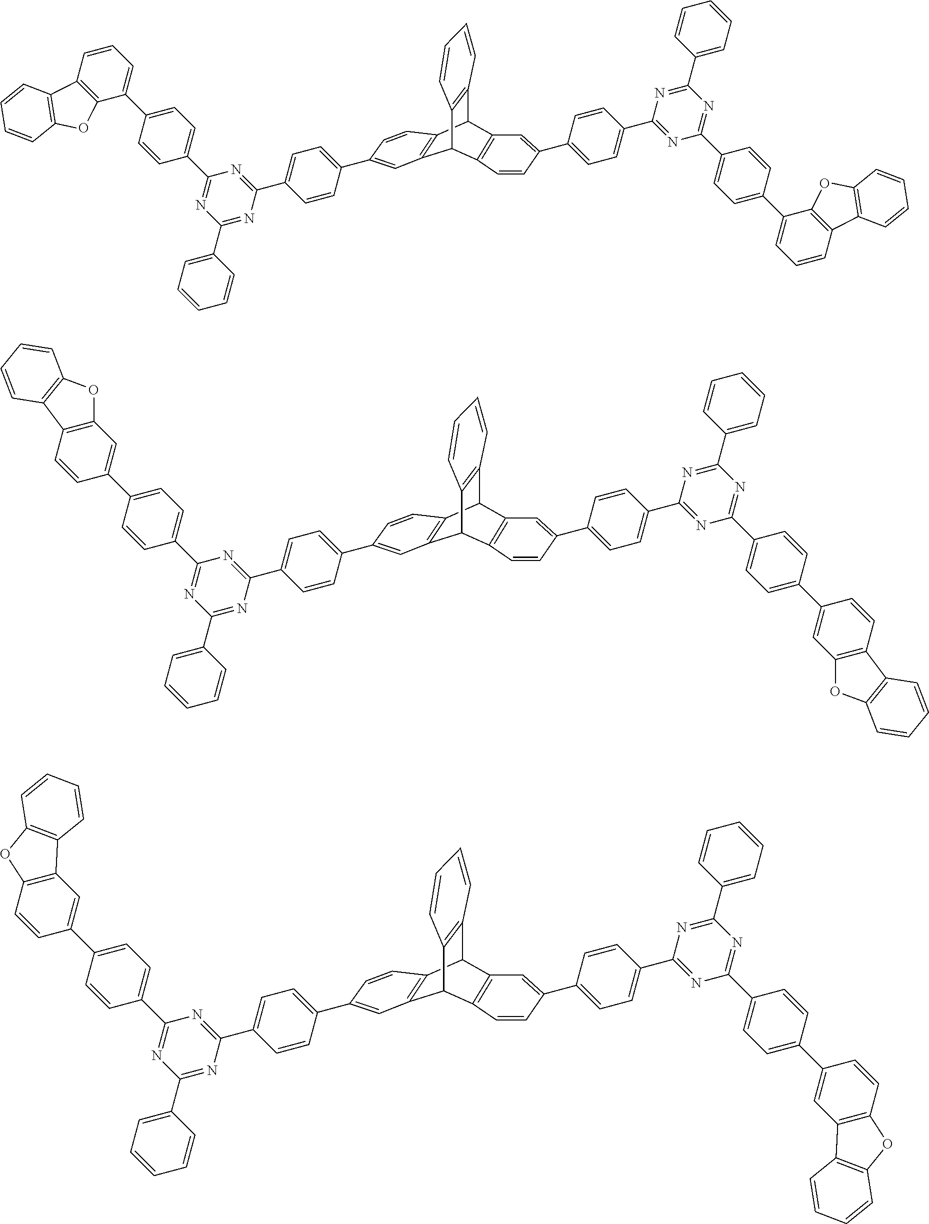

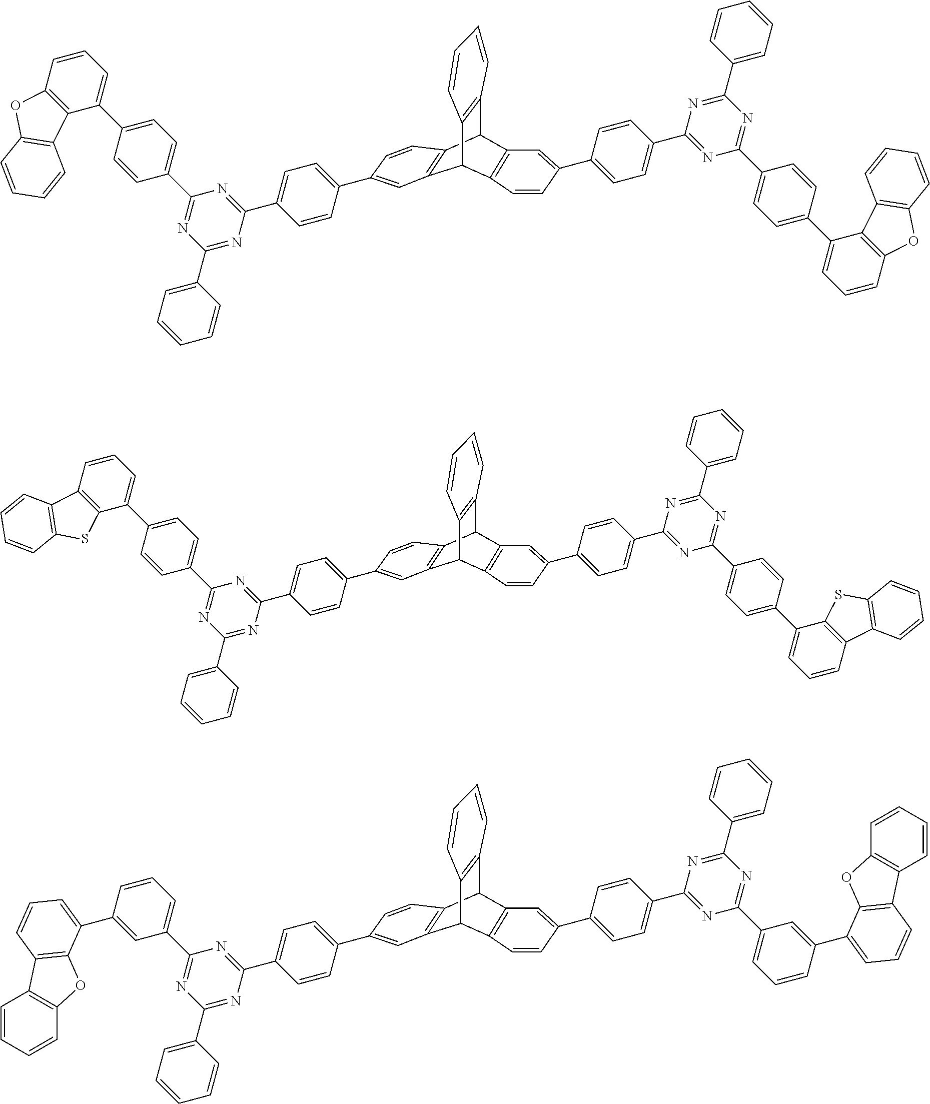

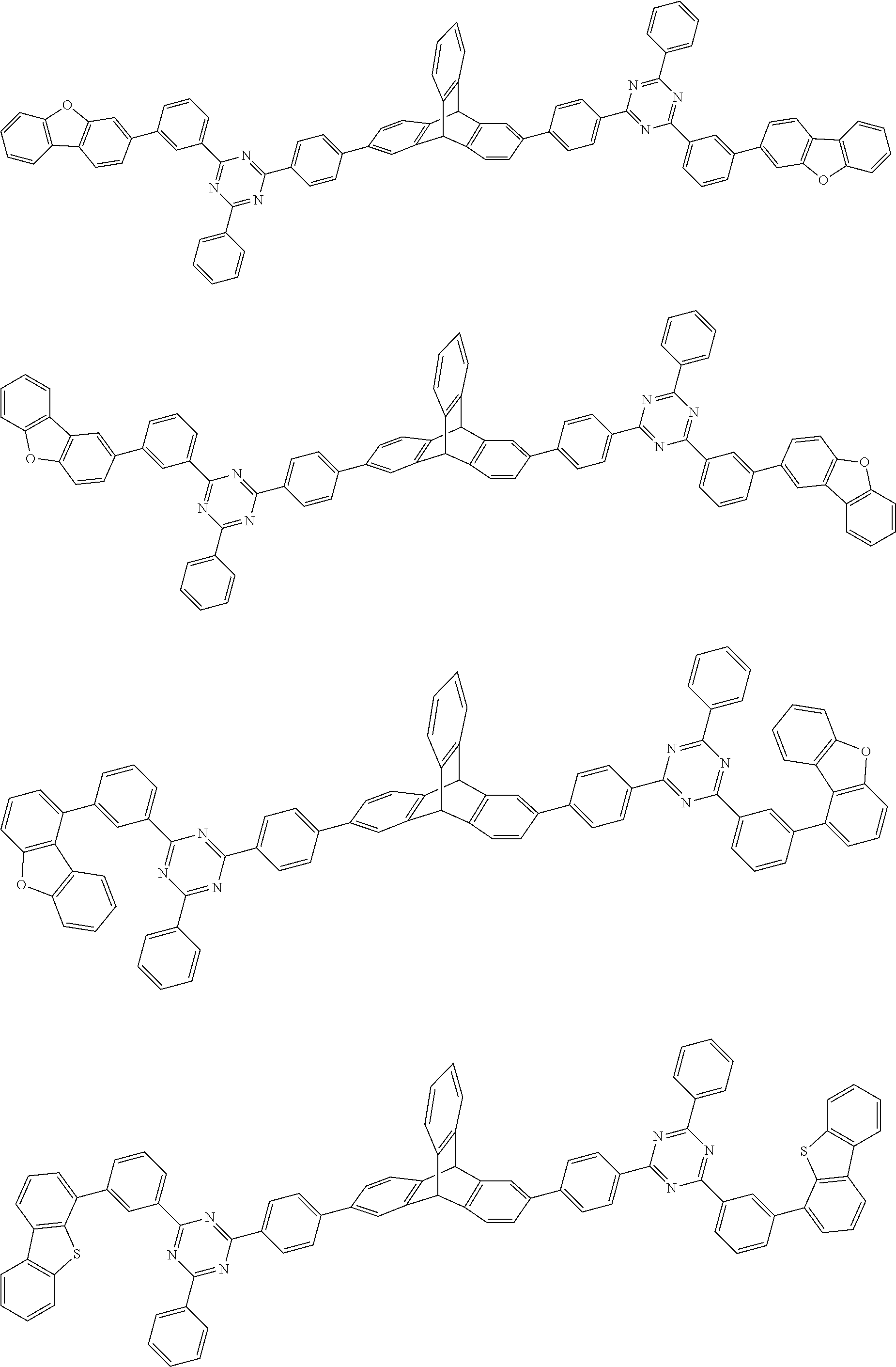

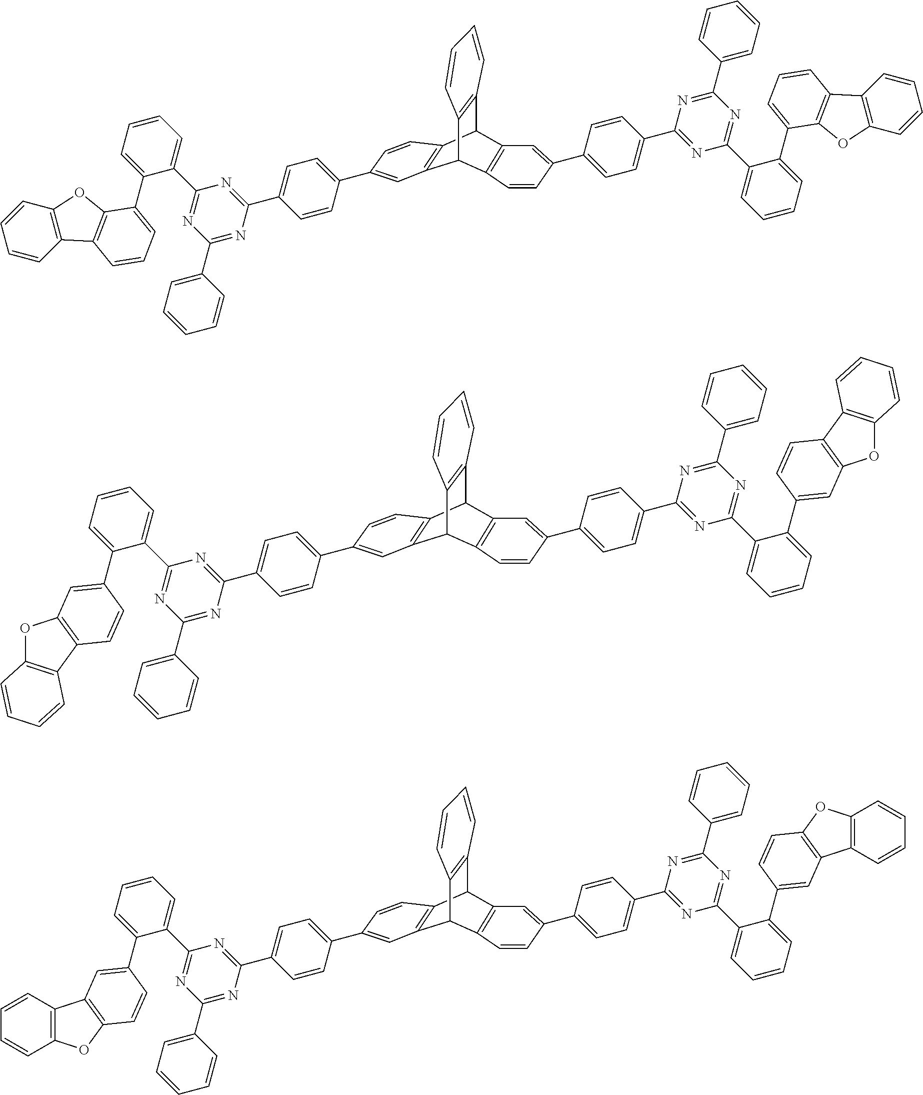 [SMILES: c1ccc(-c2nc(-c3ccc(-c4ccc5c(c4)C4c6ccccc6C5c5cc(-c6ccc(-c7nc(-c8ccccc8)nc(-c8ccccc8-c8cccc9c8oc8ccccc89)n7)cc6)ccc54)cc3)nc(-c3ccccc3-c3cccc4c3oc3ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8ccccc8-c8ccc9c(c8)oc8ccccc89)n7)cc6)ccc54)cc3)nc(-c3ccccc3-c3ccc4c(c3)oc3ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8ccccc8-c8ccc9oc%10ccccc%10c9c8)n7)cc6)ccc54)cc3)nc(-c3ccccc3-c3ccc4oc5ccccc5c4c3)n2)cc1]

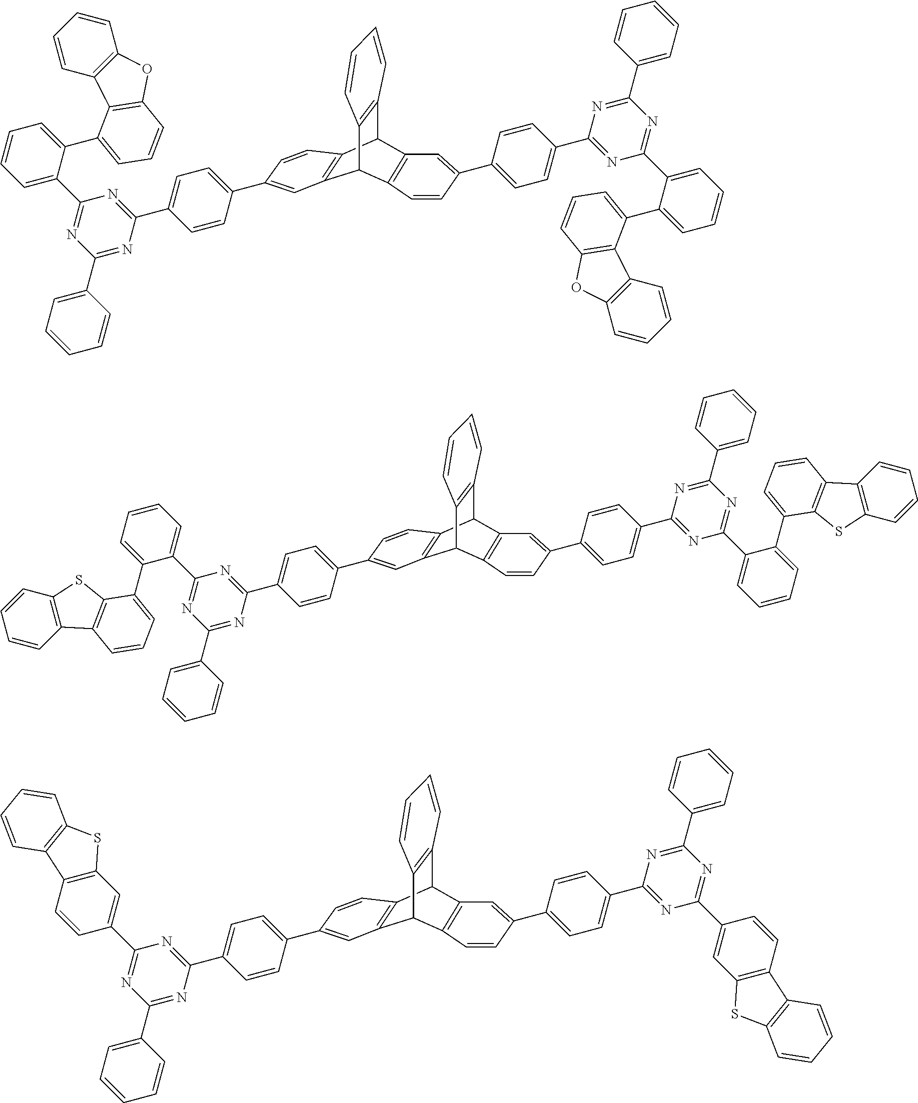 [SMILES: c1ccc(-c2nc(-c3ccc(-c4ccc5c(c4)C4c6ccccc6C5c5cc(-c6ccc(-c7nc(-c8ccccc8)nc(-c8ccccc8-c8cccc9oc%10ccccc%10c89)n7)cc6)ccc54)cc3)nc(-c3ccccc3-c3cccc4oc5ccccc5c34)n2)cc1 ; c1ccc(-c2nc(-c3ccc(-c4ccc5c(c4)C4c6ccccc6C5c5cc(-c6ccc(-c7nc(-c8ccccc8)nc(-c8ccccc8-c8cccc9c8sc8ccccc89)n7)cc6)ccc54)cc3)nc(-c3ccccc3-c3cccc4c3sc3ccccc34)n2)cc1 ; c1ccc(-c2nc(-c3ccc(-c4ccc5c(c4)C4c6ccccc6C5c5cc(-c6ccc(-c7nc(-c8ccccc8)nc(-c8ccc9c(c8)sc8ccccc89)n7)cc6)ccc54)cc3)nc(-c3ccc4c(c3)sc3ccccc34)n2)cc1]

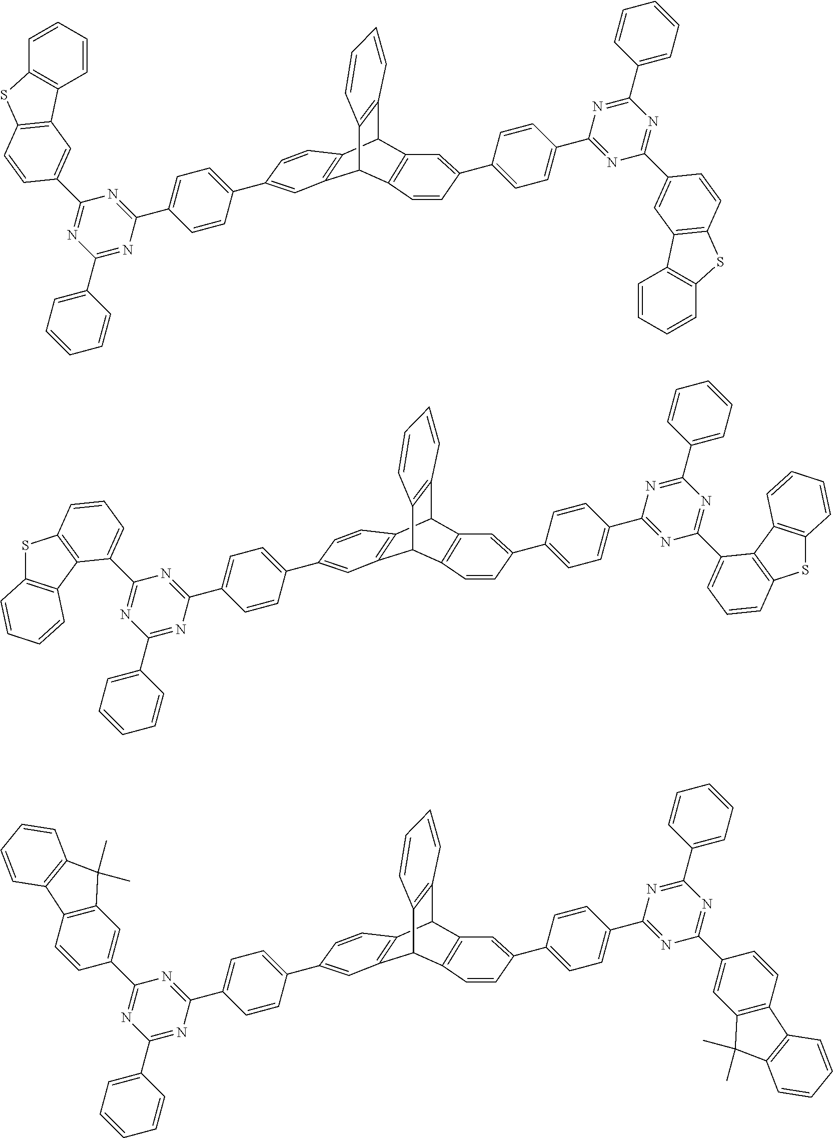 [SMILES: c1ccc(-c2nc(-c3ccc(-c4ccc5c(c4)C4c6ccccc6C5c5cc(-c6ccc(-c7nc(-c8ccccc8)nc(-c8ccc9sc%10ccccc%10c9c8)n7)cc6)ccc54)cc3)nc(-c3ccc4sc5ccccc5c4c3)n2)cc1 ; c1ccc(-c2nc(-c3ccc(-c4ccc5c(c4)C4c6ccccc6C5c5cc(-c6ccc(-c7nc(-c8ccccc8)nc(-c8cccc9sc%10ccccc%10c89)n7)cc6)ccc54)cc3)nc(-c3cccc4sc5ccccc5c34)n2)cc1 ; CC1(C)c2ccccc2-c2ccc(-c3nc(-c4ccccc4)nc(-c4ccc(-c5ccc6c(c5)C5c7ccccc7C6c6cc(-c7ccc(-c8nc(-c9ccccc9)nc(-c9ccc%10c(c9)C(C)(C)c9ccccc9-%10)n8)cc7)ccc65)cc4)n3)cc21]

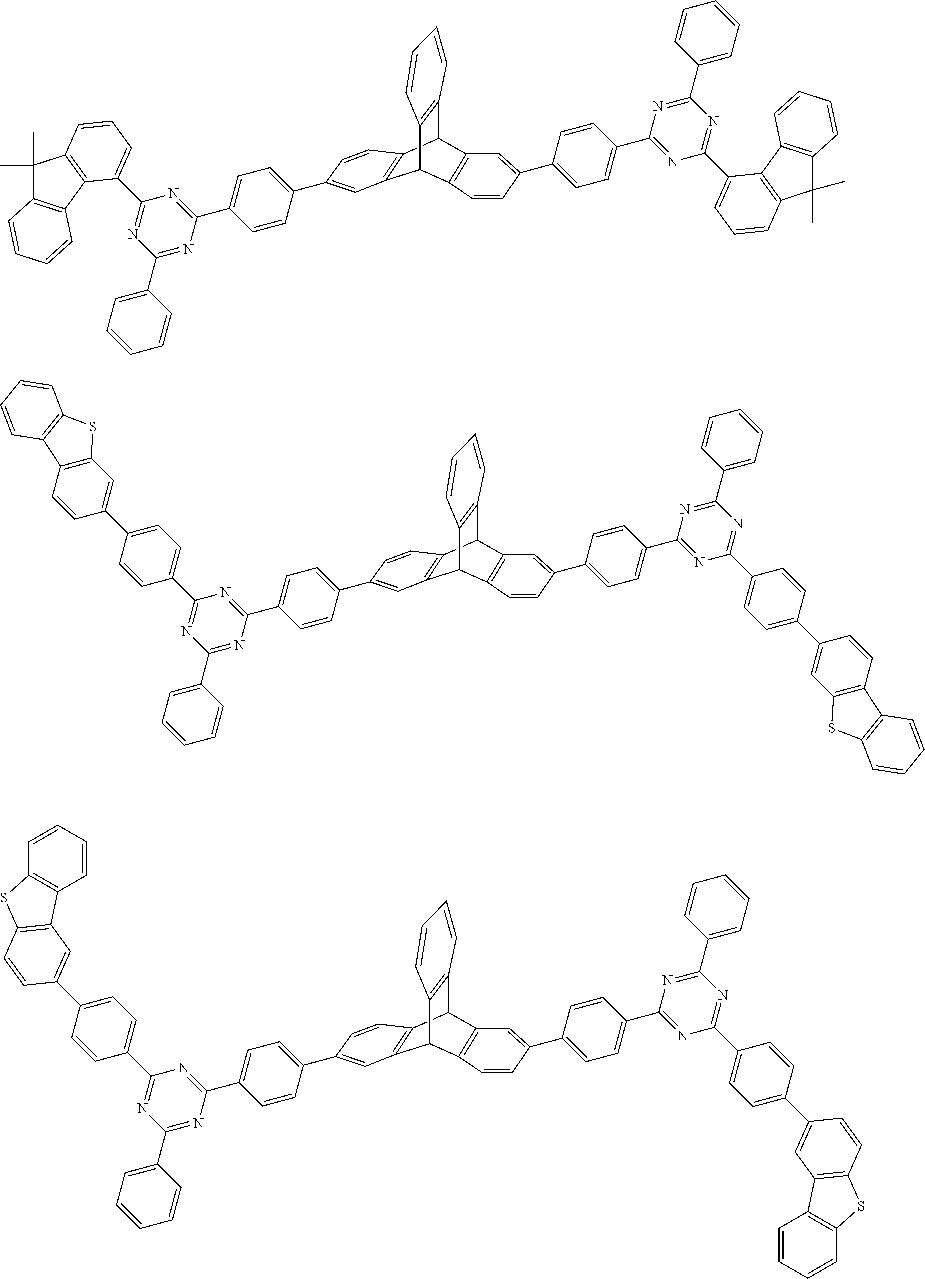

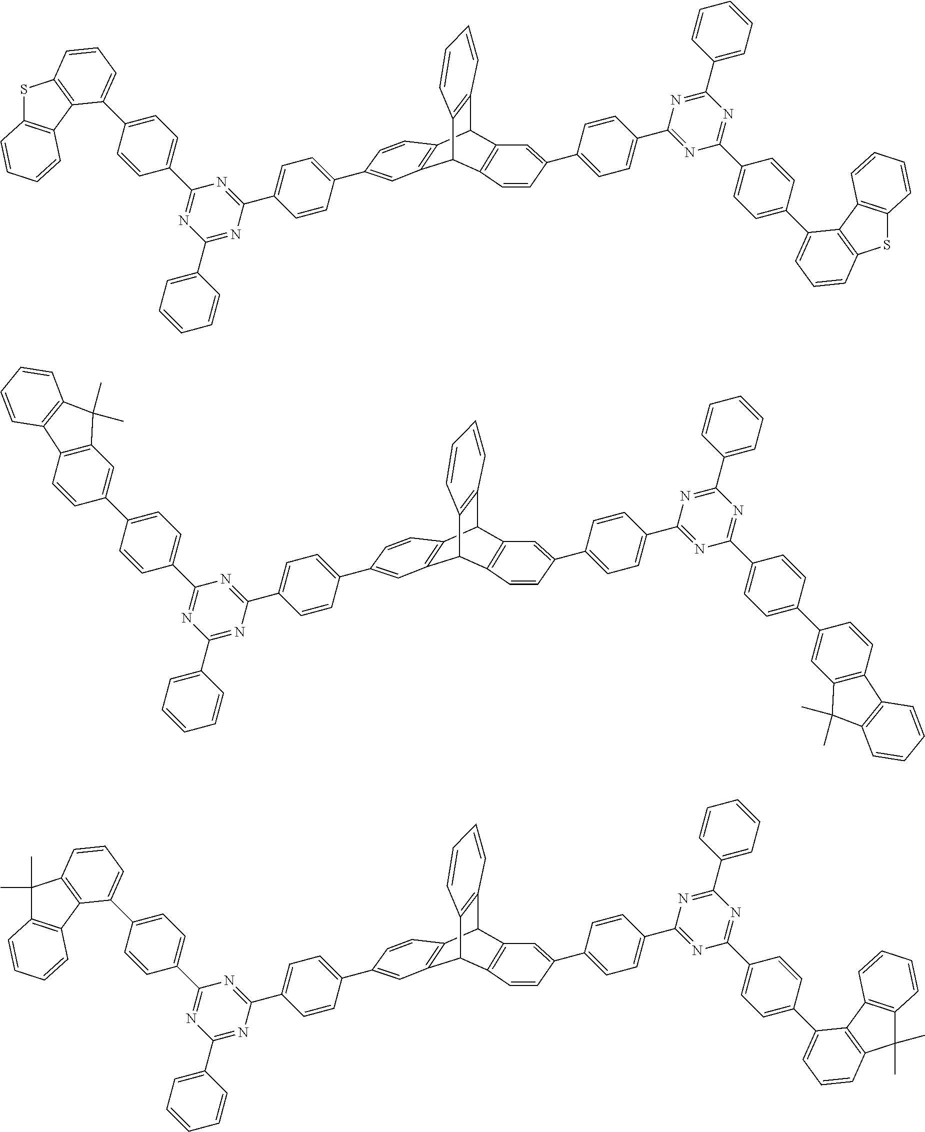

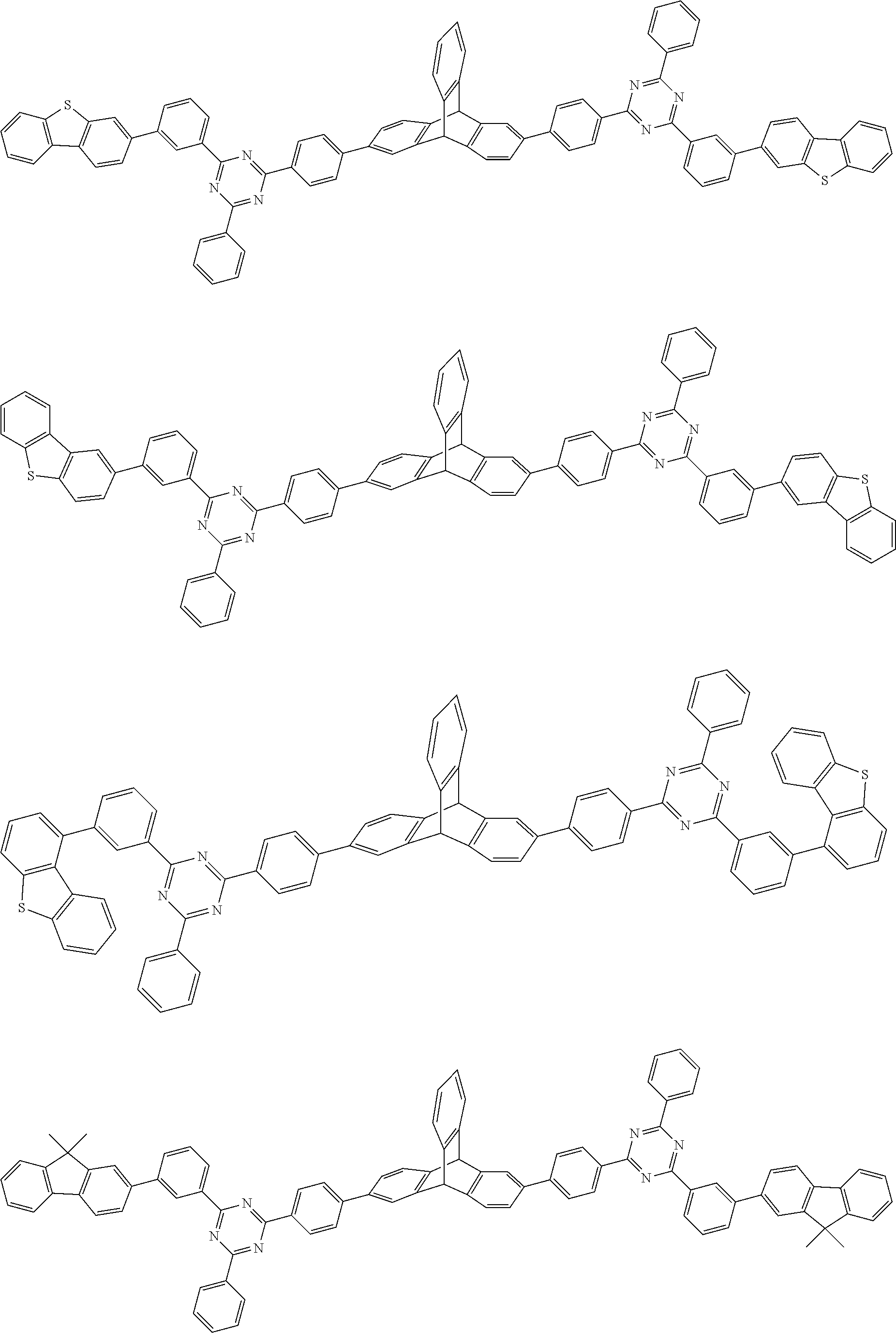

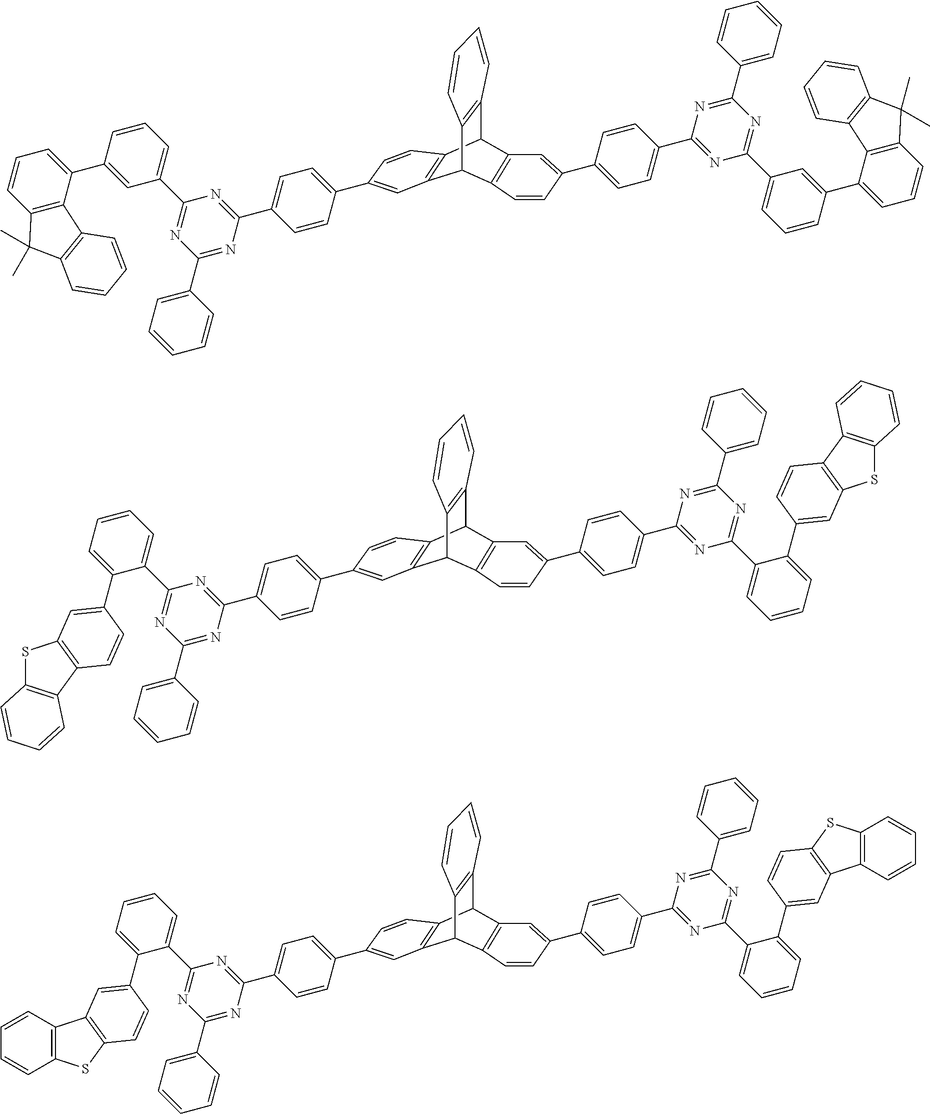

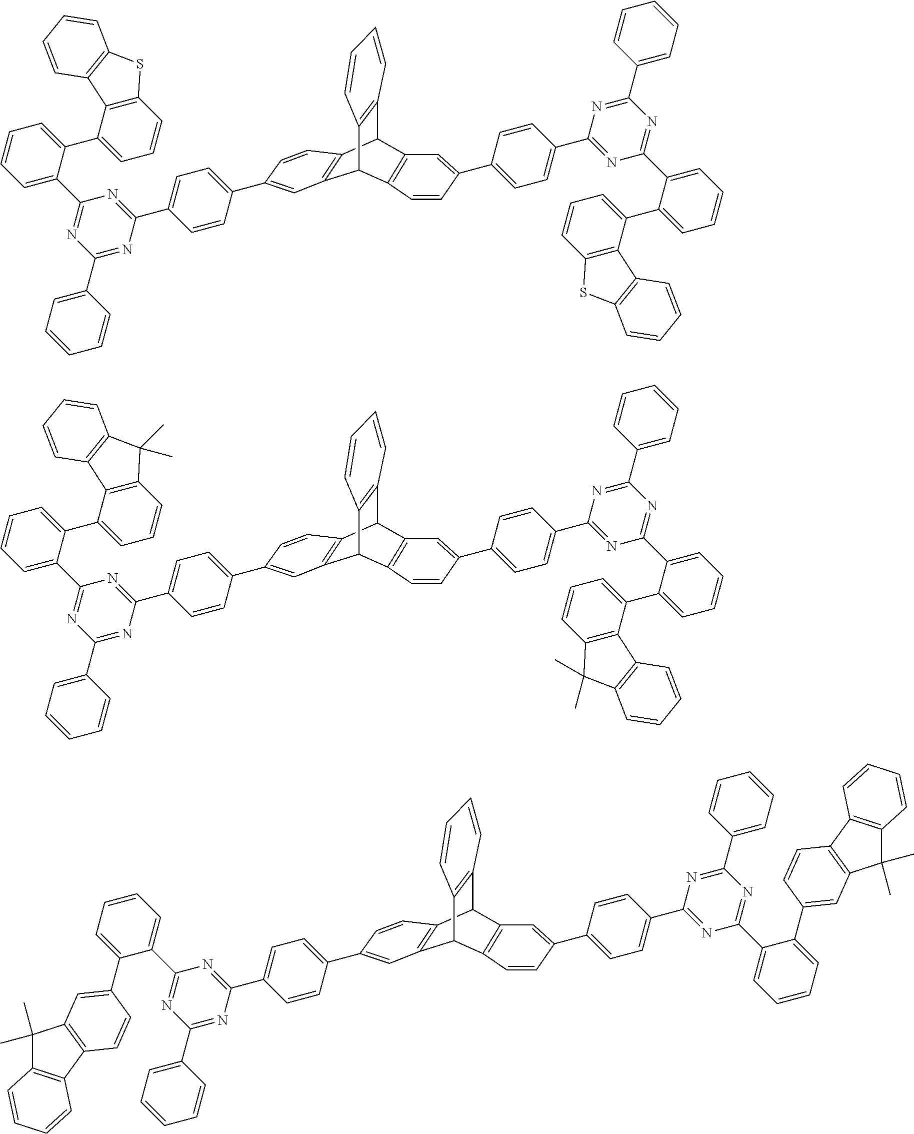

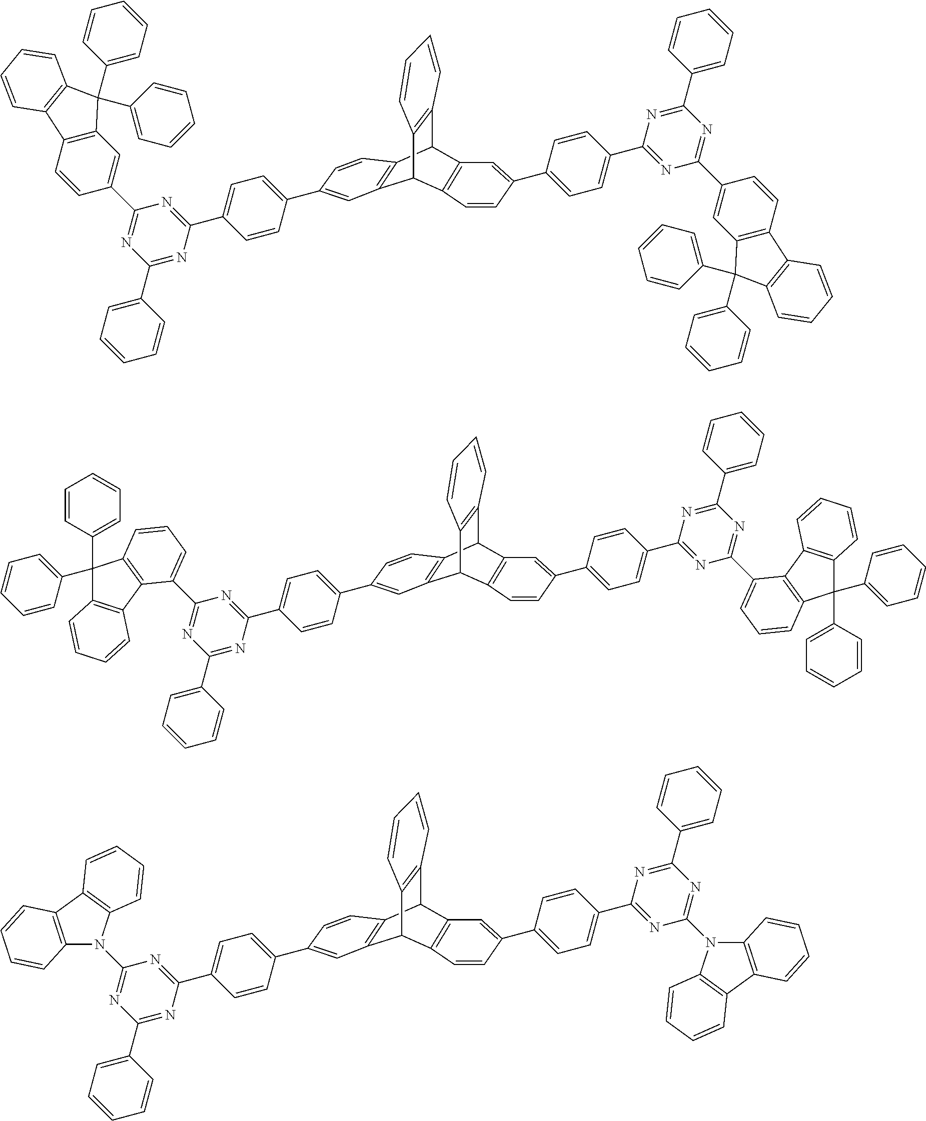

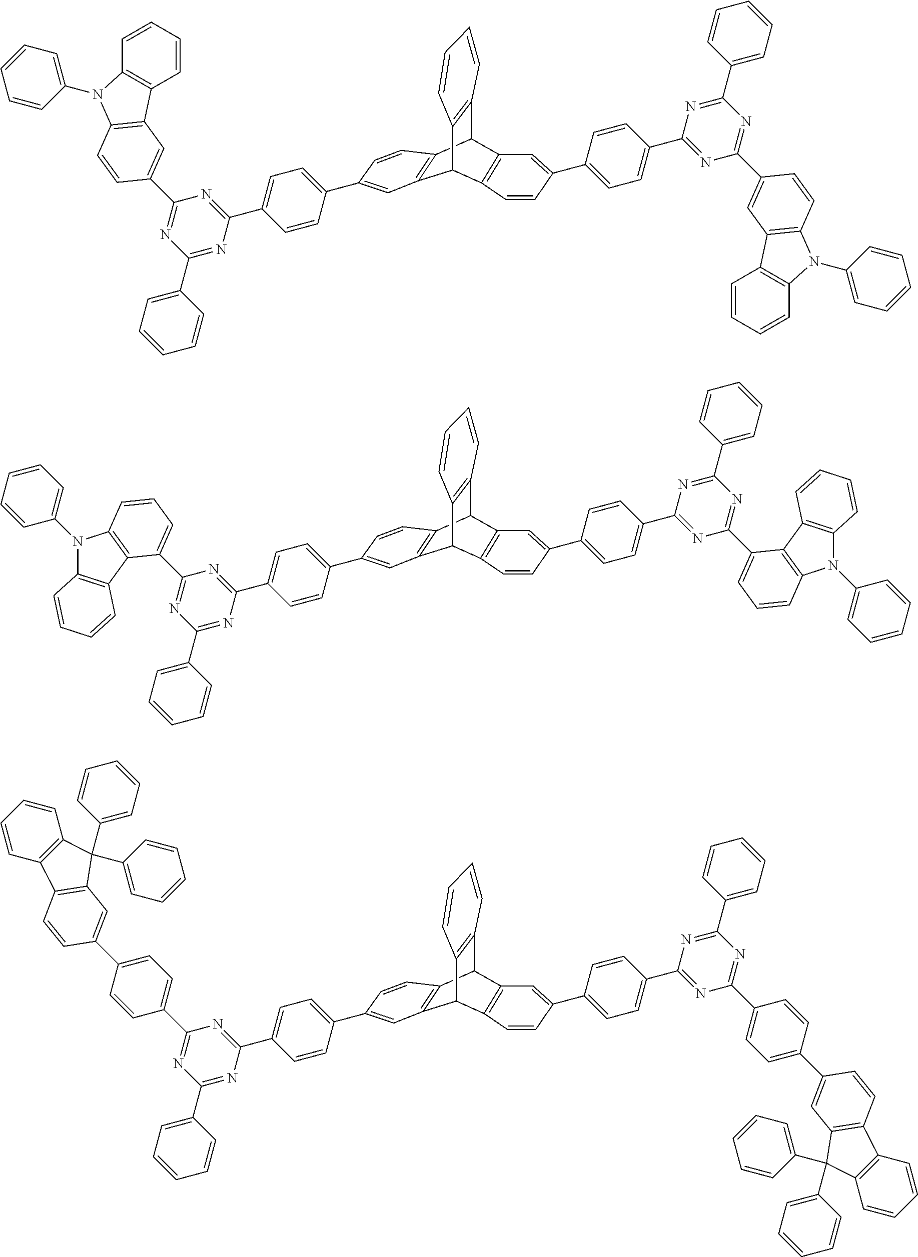

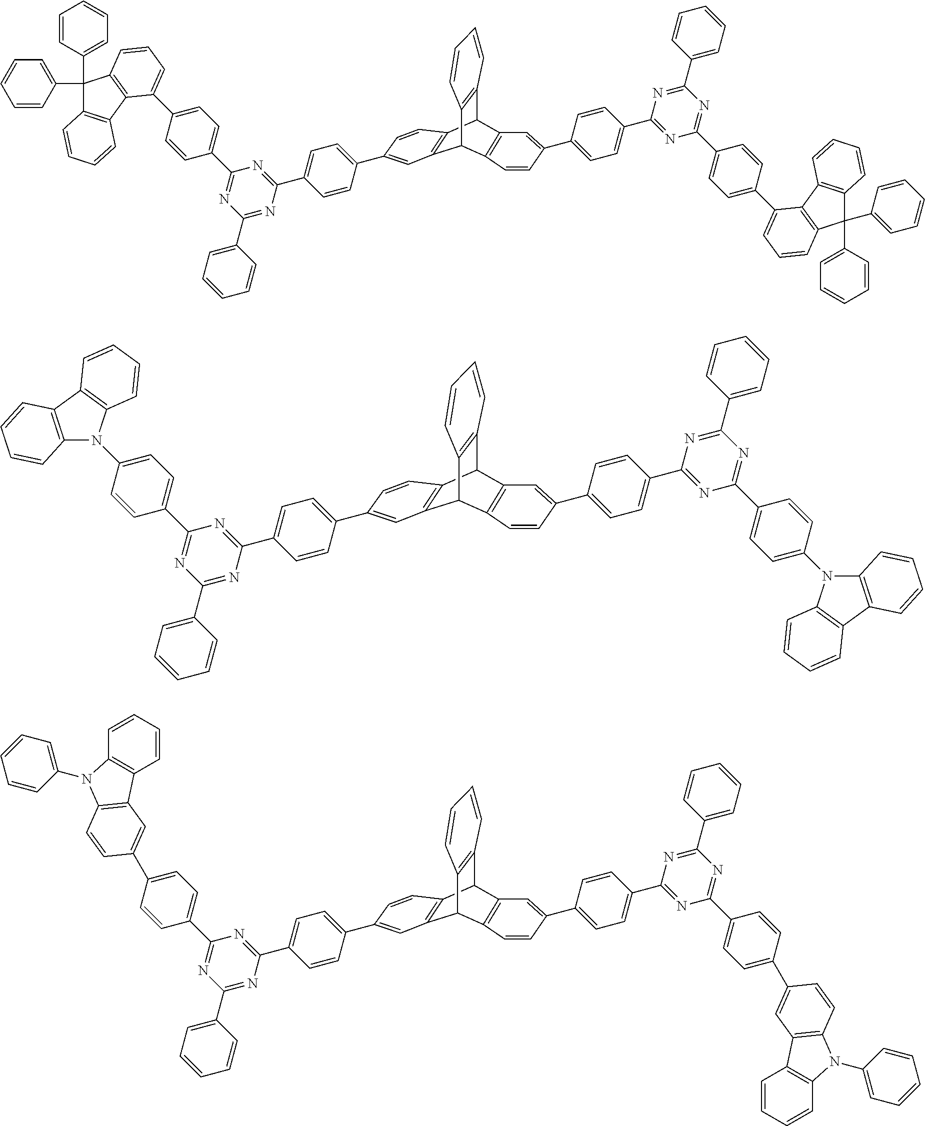

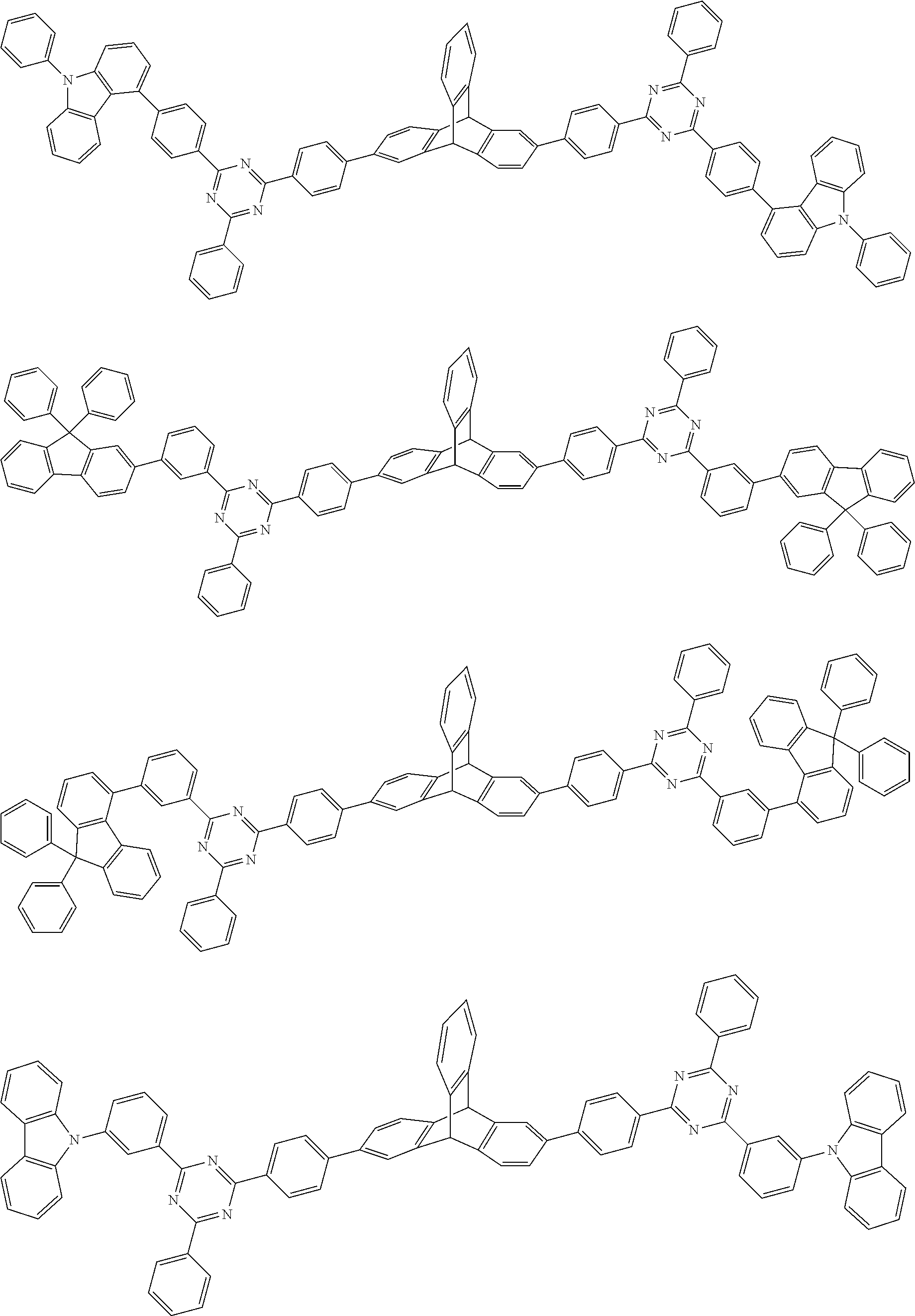

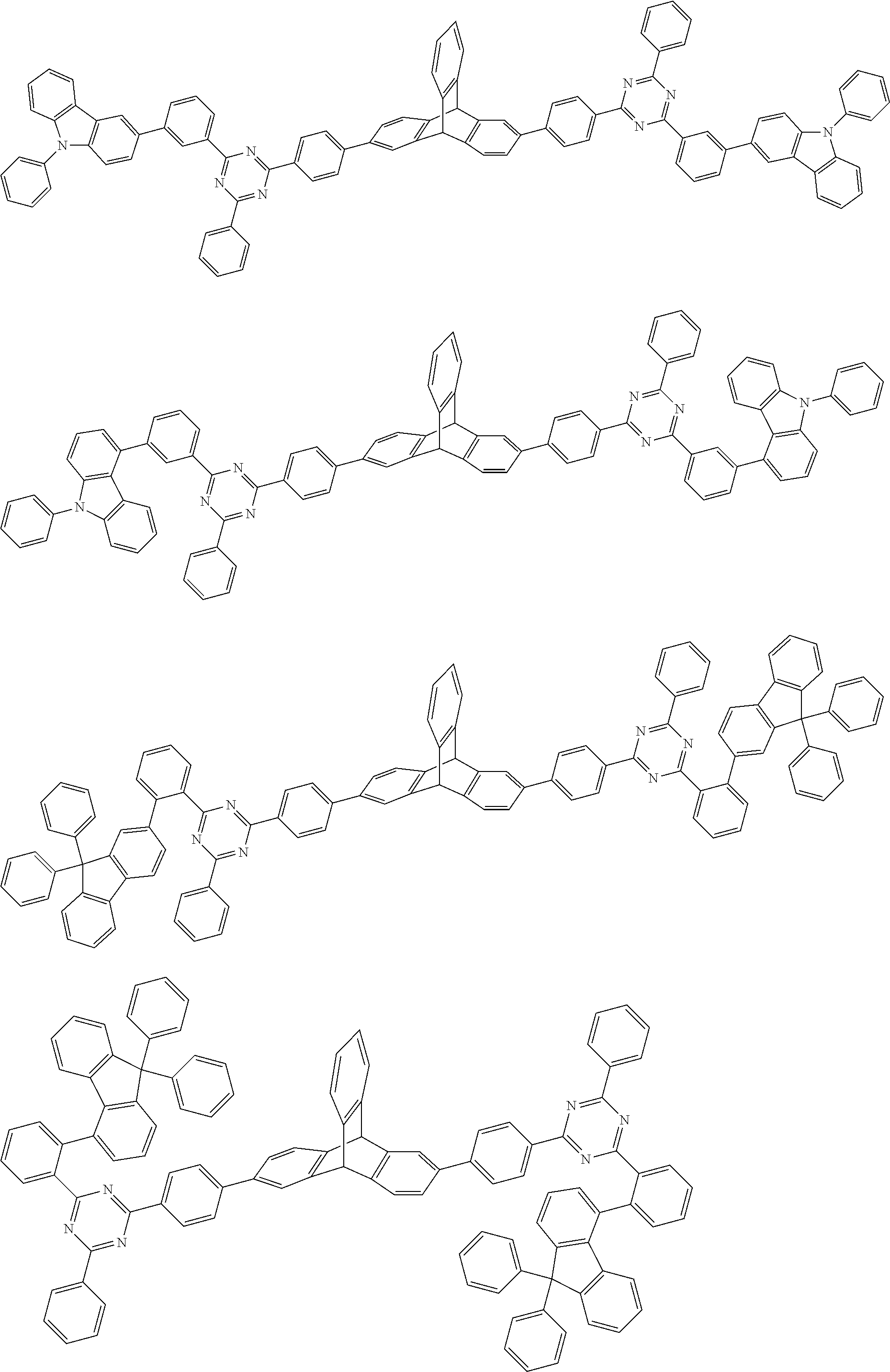

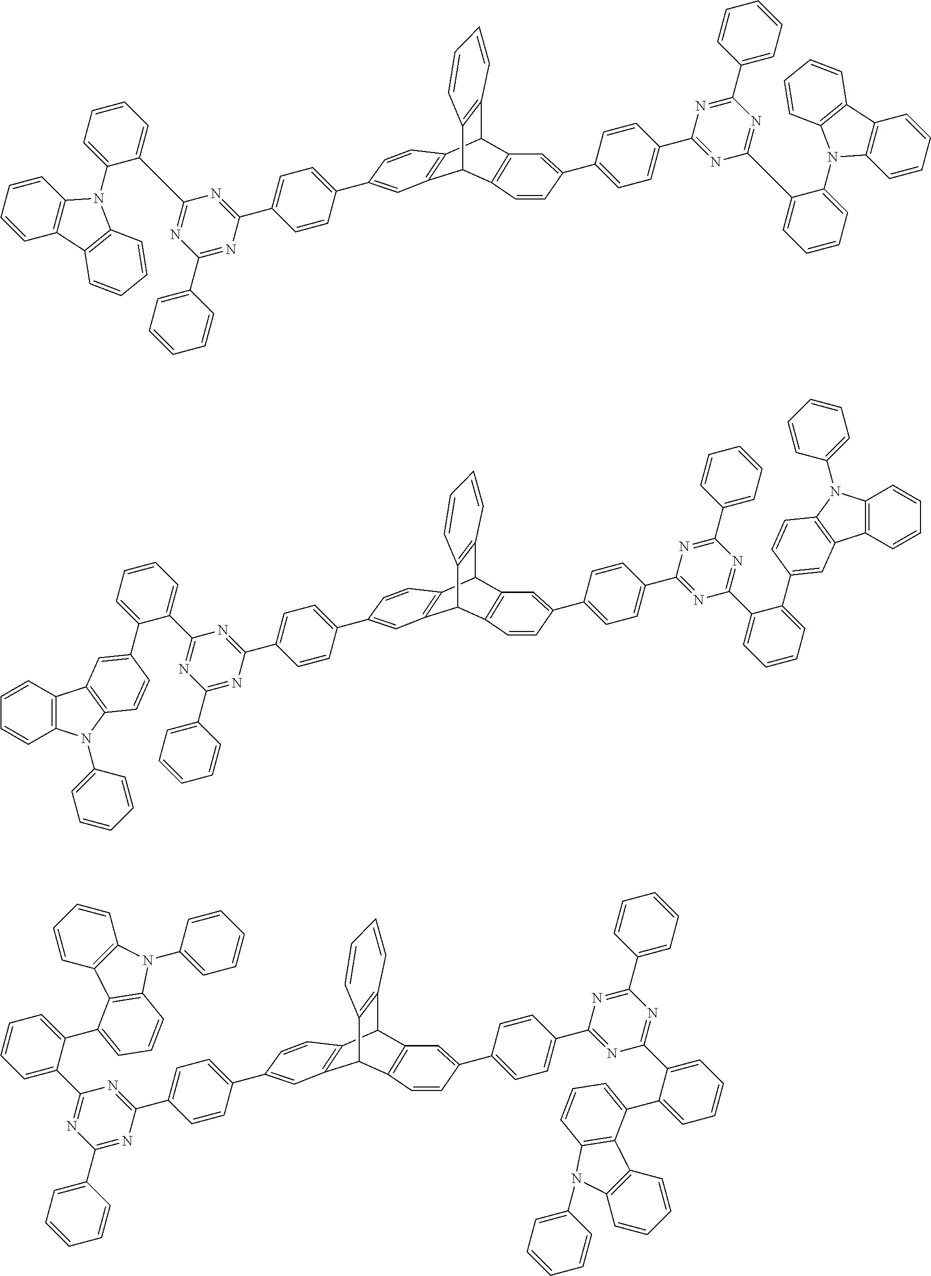

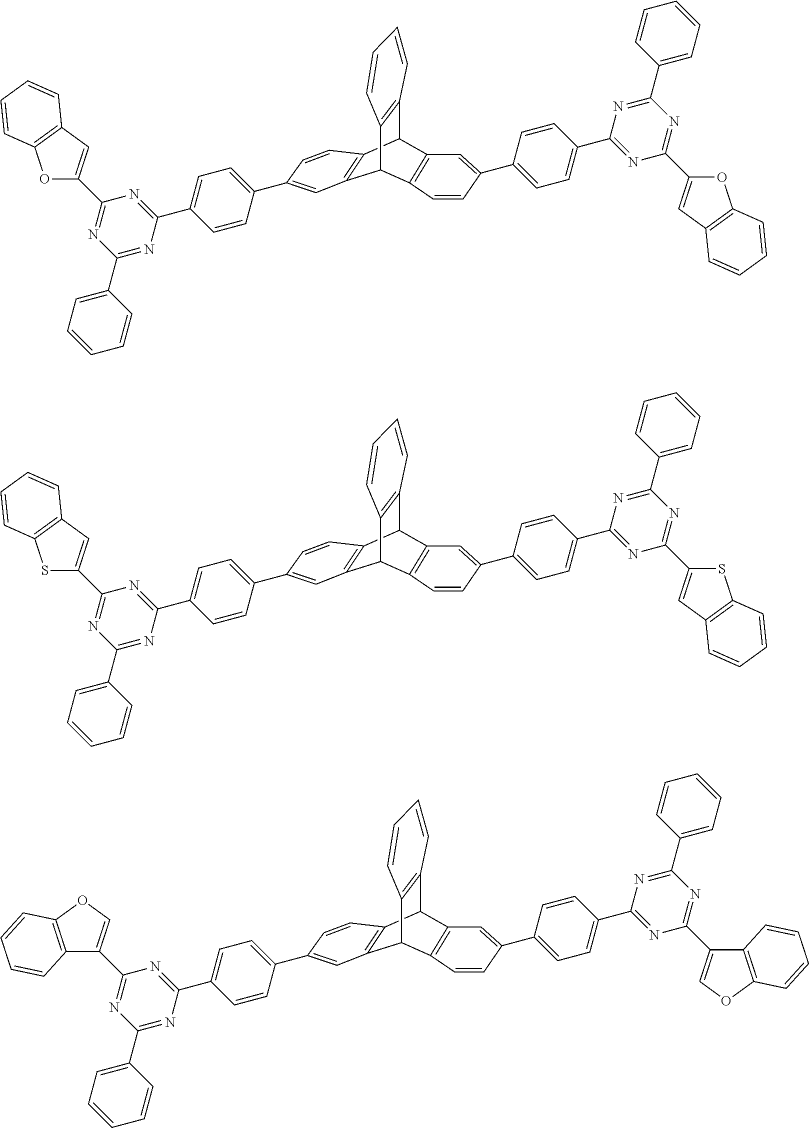 [SMILES: c1ccc(-c2nc(-c3ccc(-c4ccc5c(c4)C4c6ccccc6C5c5cc(-c6ccc(-c7nc(-c8ccccc8)nc(-c8cc9ccccc9o8)n7)cc6)ccc54)cc3)nc(-c3cc4ccccc4o3)n2)cc1 ; c1ccc(-c2nc(-c3ccc(-c4ccc5c(c4)C4c6ccccc6C5c5cc(-c6ccc(-c7nc(-c8ccccc8)nc(-c8cc9ccccc9s8)n7)cc6)ccc54)cc3)nc(-c3cc4ccccc4s3)n2)cc1 ; c1ccc(-c2nc(-c3ccc(-c4ccc5c(c4)C4c6ccccc6C5c5cc(-c6ccc(-c7nc(-c8ccccc8)nc(-c8coc9ccccc89)n7)cc6)ccc54)cc3)nc(-c3coc4ccccc34)n2)cc1]

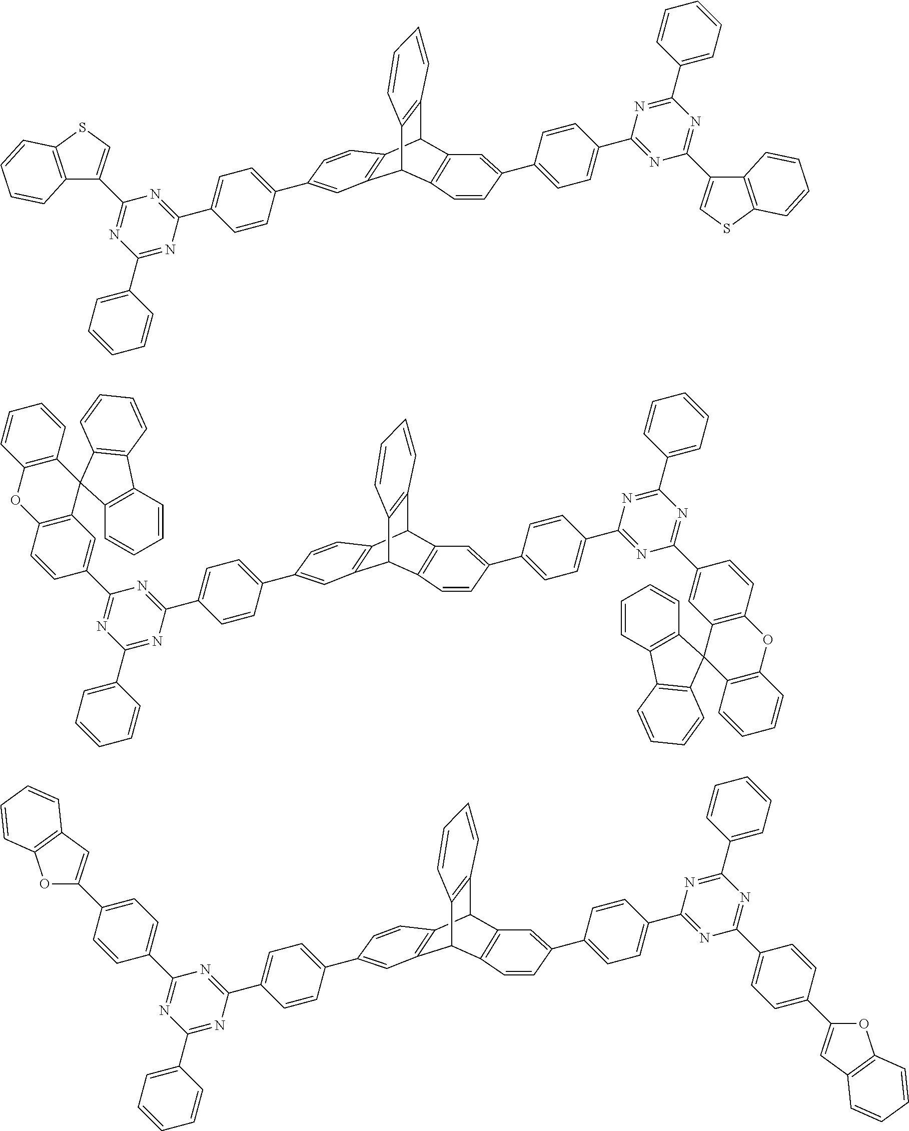

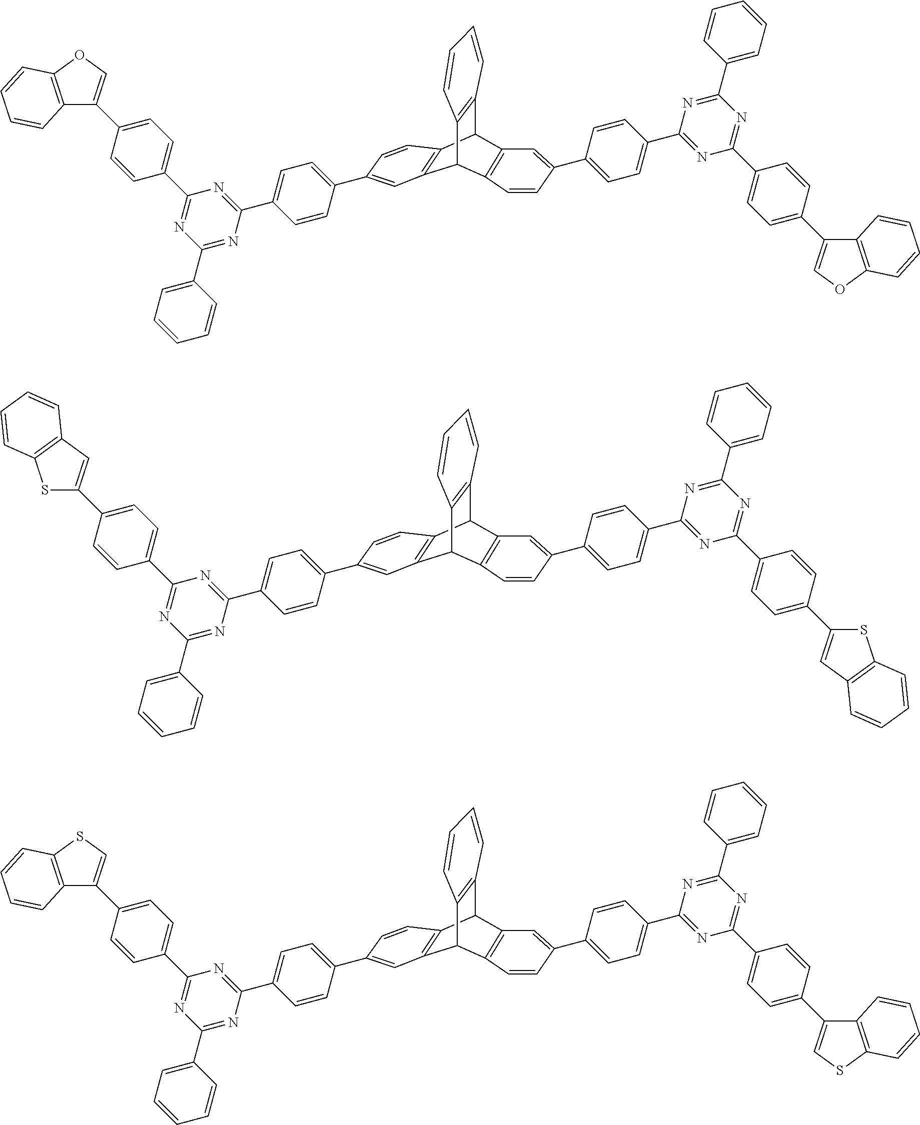 [SMILES: c1ccc(-c2nc(-c3ccc(-c4ccc5c(c4)C4c6ccccc6C5c5cc(-c6ccc(-c7nc(-c8ccccc8)nc(-c8ccc(-c9coc%10ccccc9%10)cc8)n7)cc6)ccc54)cc3)nc(-c3ccc(-c4coc5ccccc45)cc3)n2)cc1 ; c1ccc(-c2nc(-c3ccc(-c4ccc5c(c4)C4c6ccccc6C5c5cc(-c6ccc(-c7nc(-c8ccccc8)nc(-c8ccc(-c9cc%10ccccc%10s9)cc8)n7)cc6)ccc54)cc3)nc(-c3ccc(-c4cc5ccccc5s4)cc3)n2)cc1 ; c1ccc(-c2nc(-c3ccc(-c4ccc5c(c4)C4c6ccccc6C5c5cc(-c6ccc(-c7nc(-c8ccccc8)nc(-c8ccc(-c9csc%10ccccc9%10)cc8)n7)cc6)ccc54)cc3)nc(-c3ccc(-c4csc5ccccc45)cc3)n2)cc1]

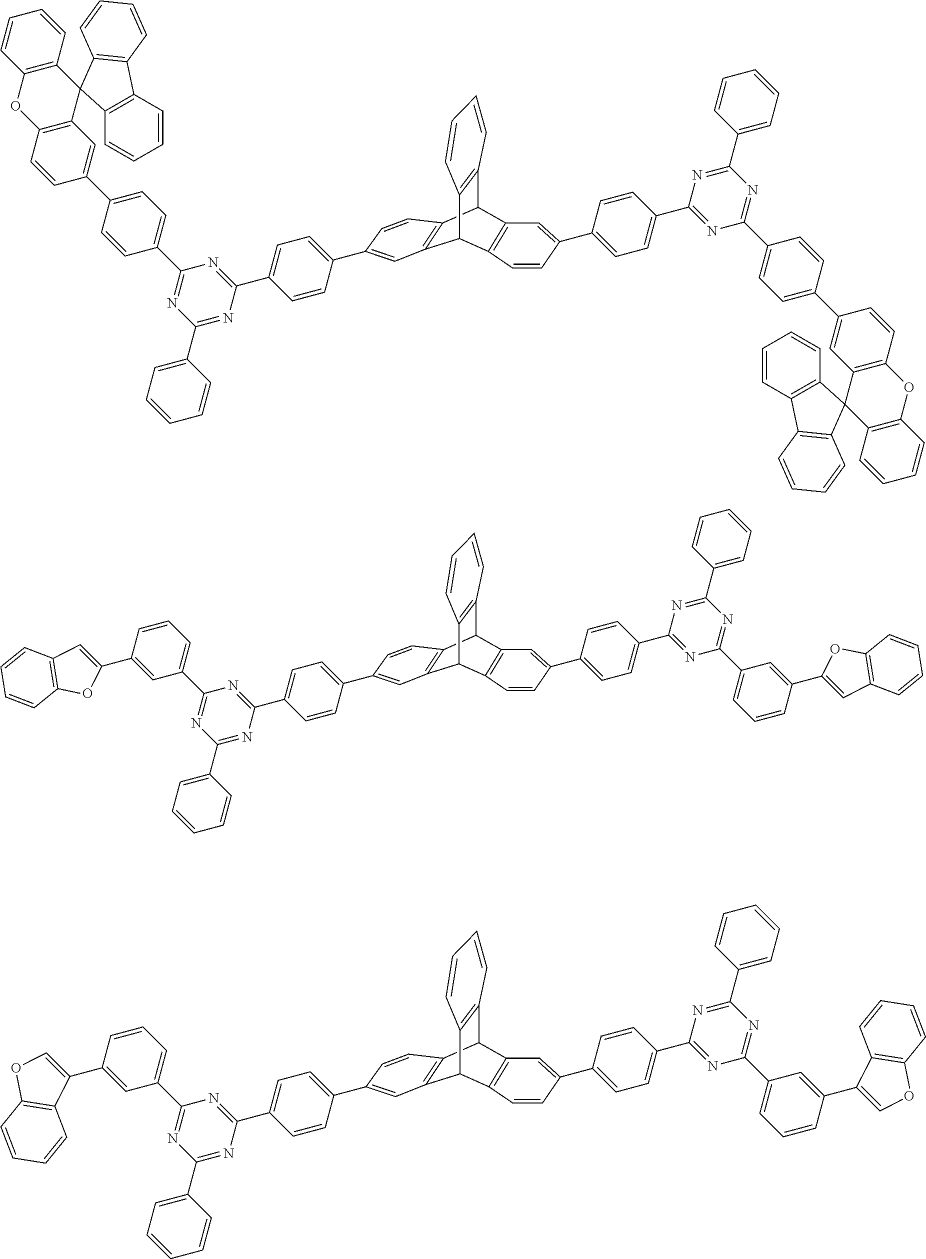

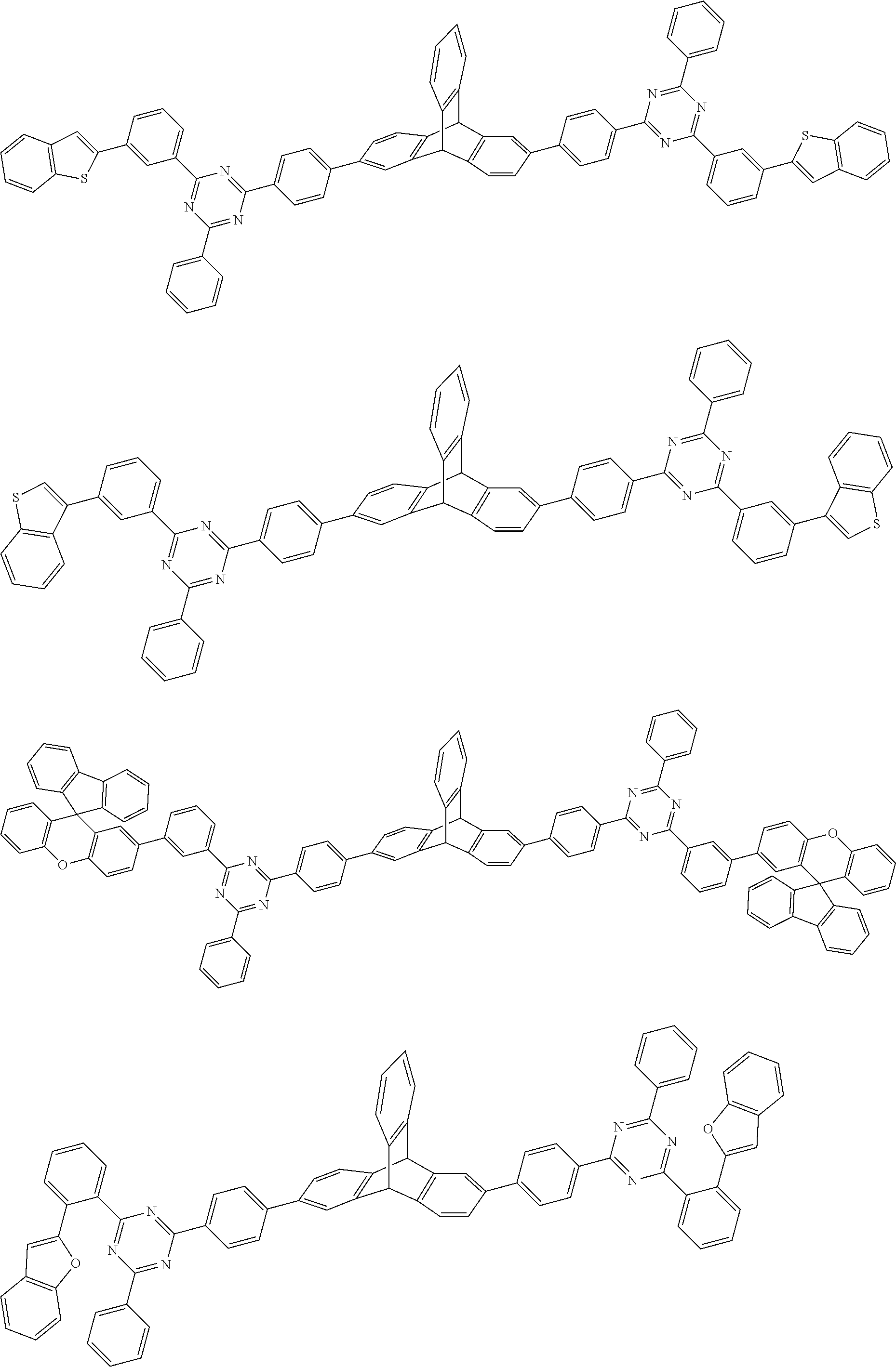

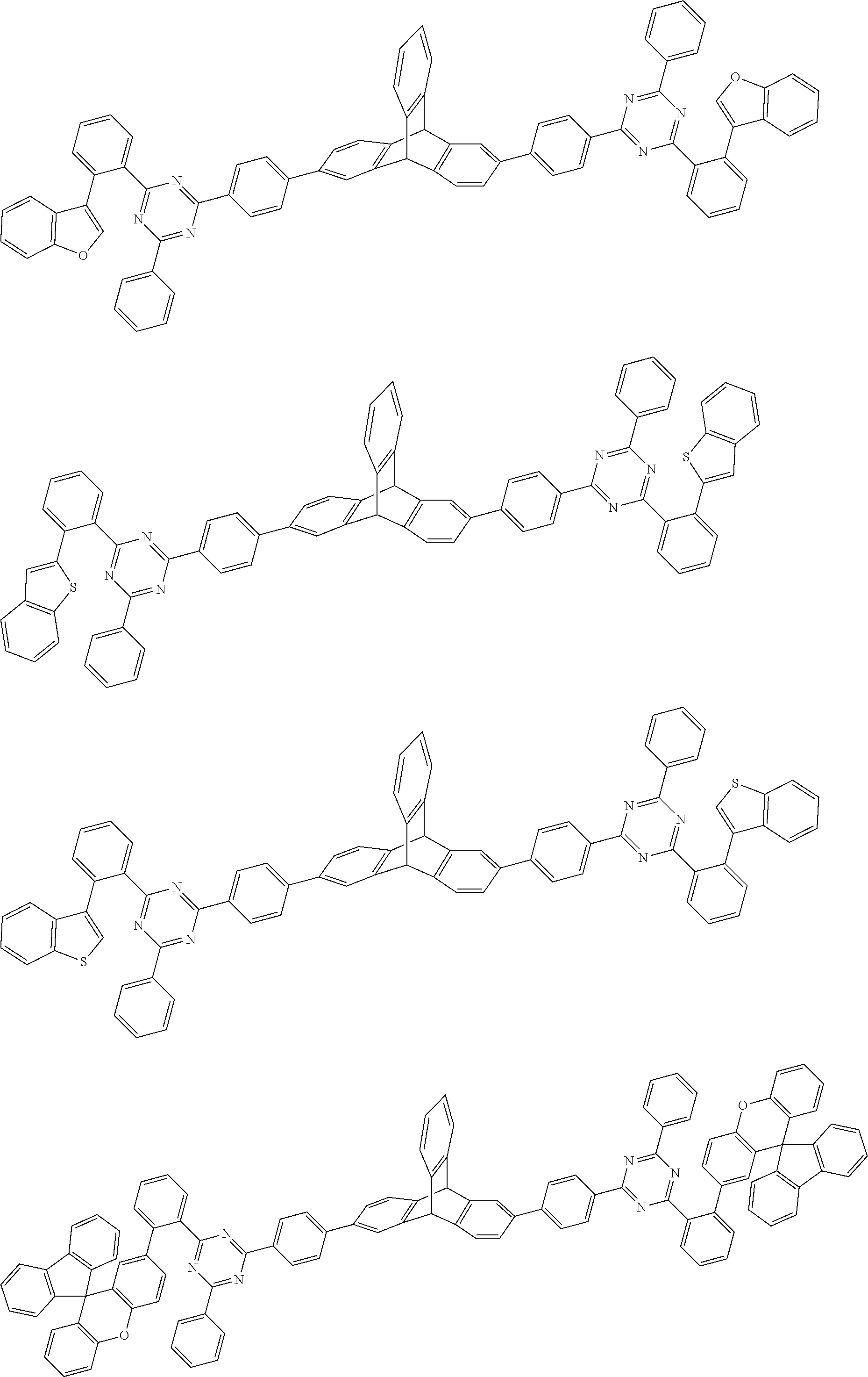

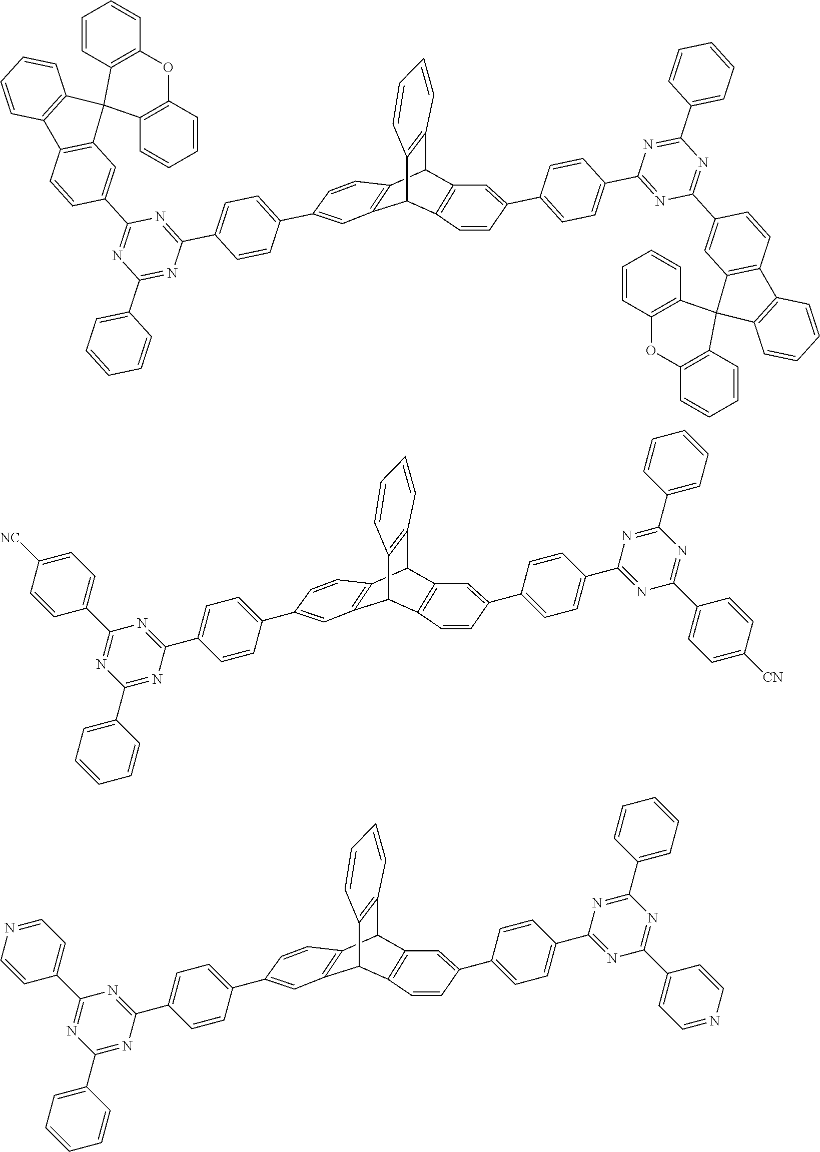

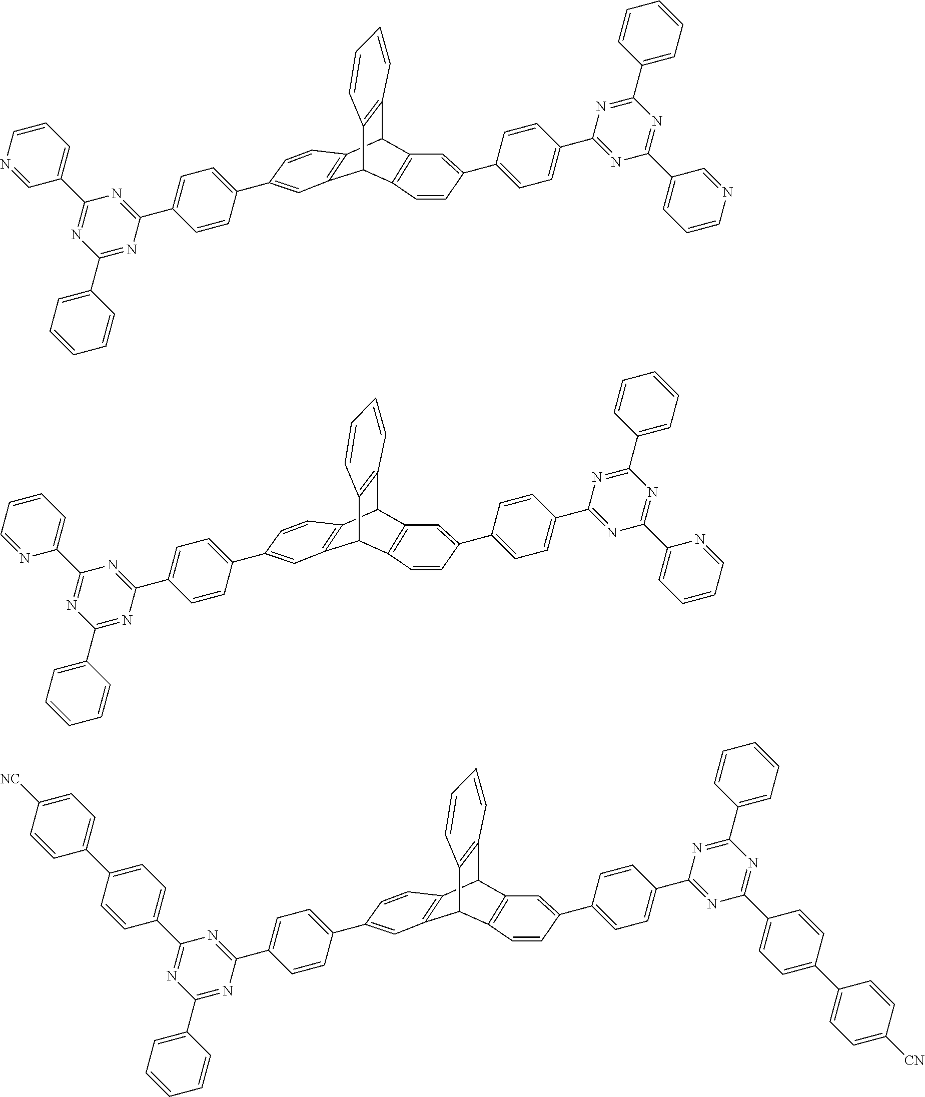 [SMILES: c1ccc(-c2nc(-c3ccc(-c4ccc5c(c4)C4c6ccccc6C5c5cc(-c6ccc(-c7nc(-c8ccccc8)nc(-c8cccnc8)n7)cc6)ccc54)cc3)nc(-c3cccnc3)n2)cc1 ; c1ccc(-c2nc(-c3ccc(-c4ccc5c(c4)C4c6ccccc6C5c5cc(-c6ccc(-c7nc(-c8ccccc8)nc(-c8ccccn8)n7)cc6)ccc54)cc3)nc(-c3ccccn3)n2)cc1 ; N#Cc1ccc(-c2ccc(-c3nc(-c4ccccc4)nc(-c4ccc(-c5ccc6c(c5)C5c7ccccc7C6c6cc(-c7ccc(-c8nc(-c9ccccc9)nc(-c9ccc(-c%10ccc(C#N)cc%10)cc9)n8)cc7)ccc65)cc4)n3)cc2)cc1]

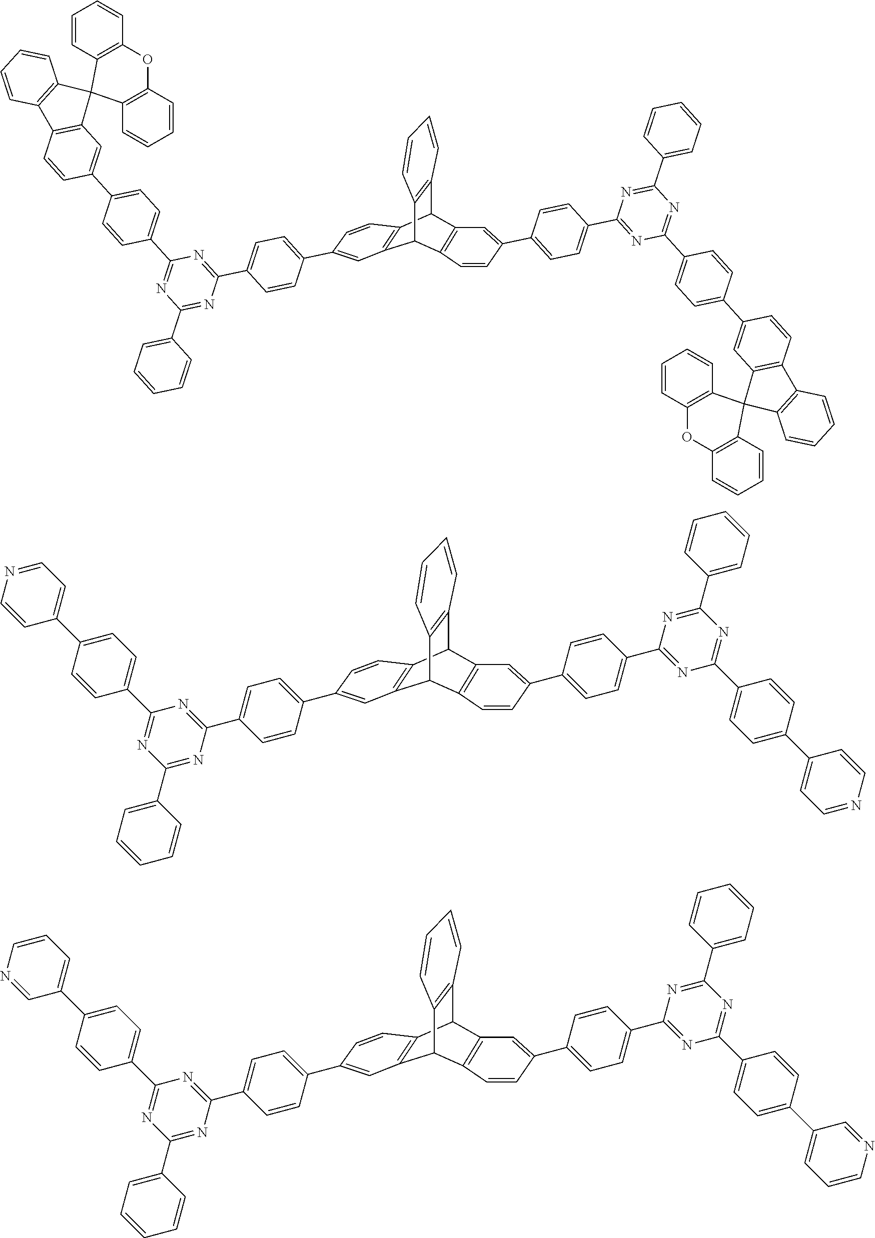

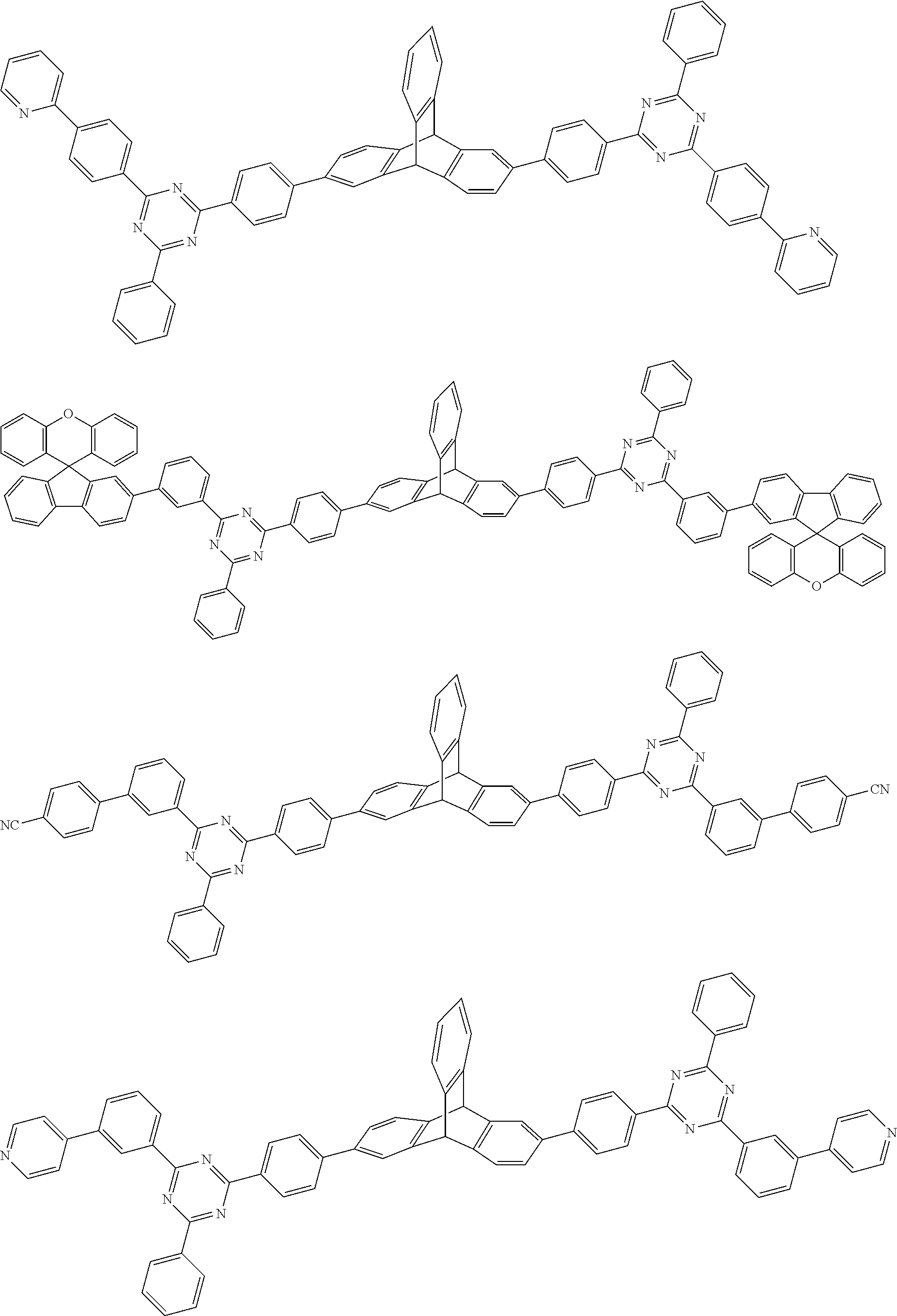

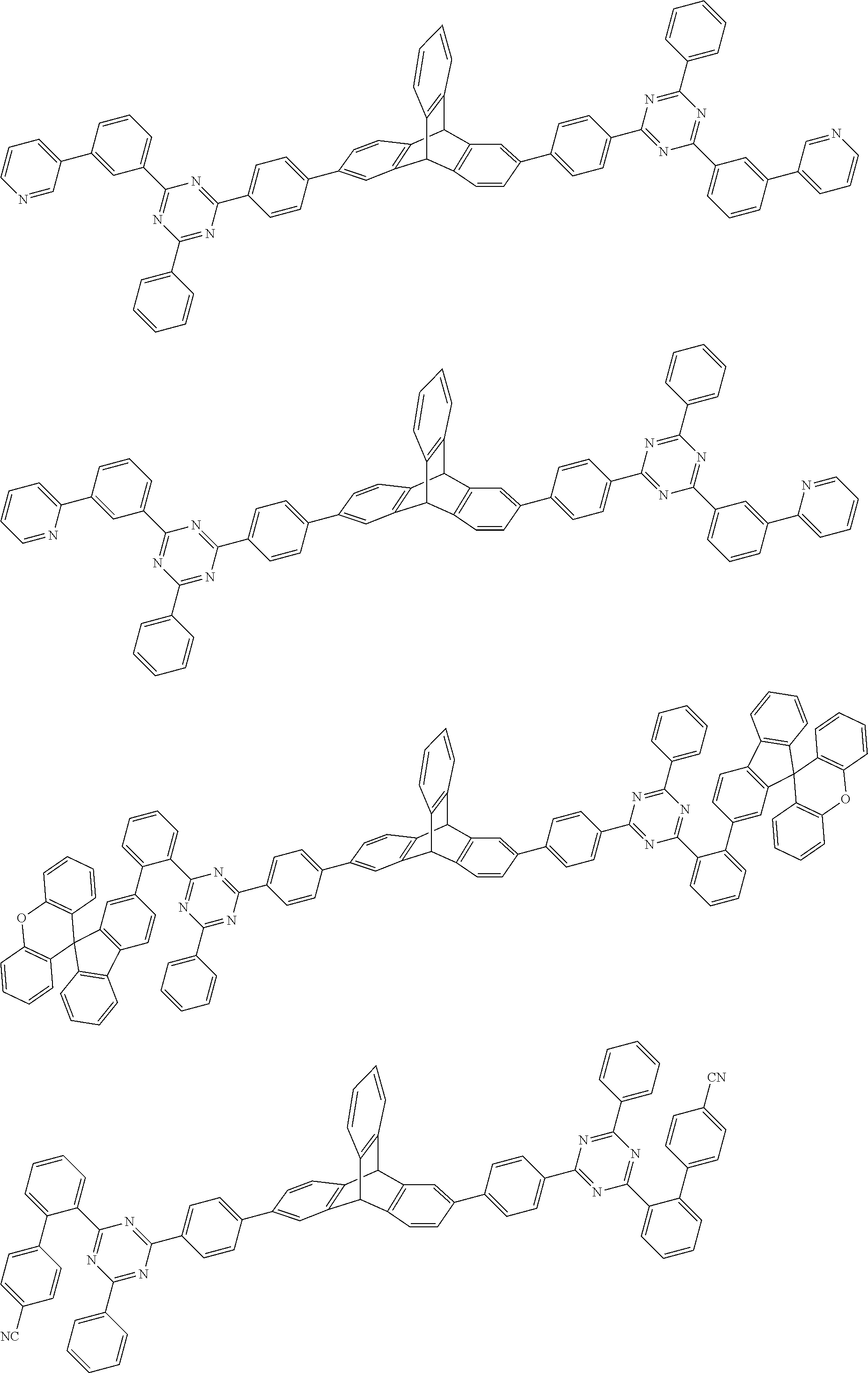

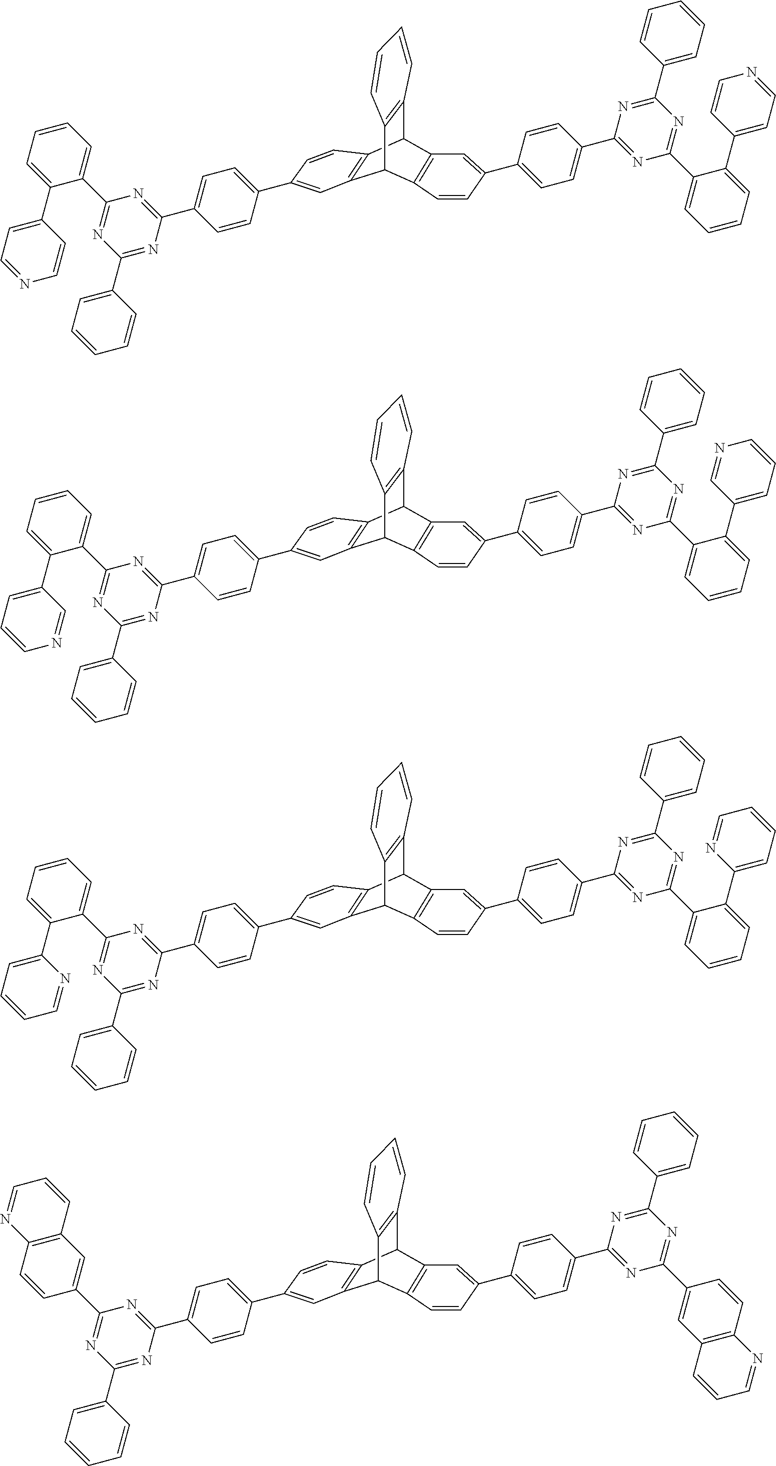

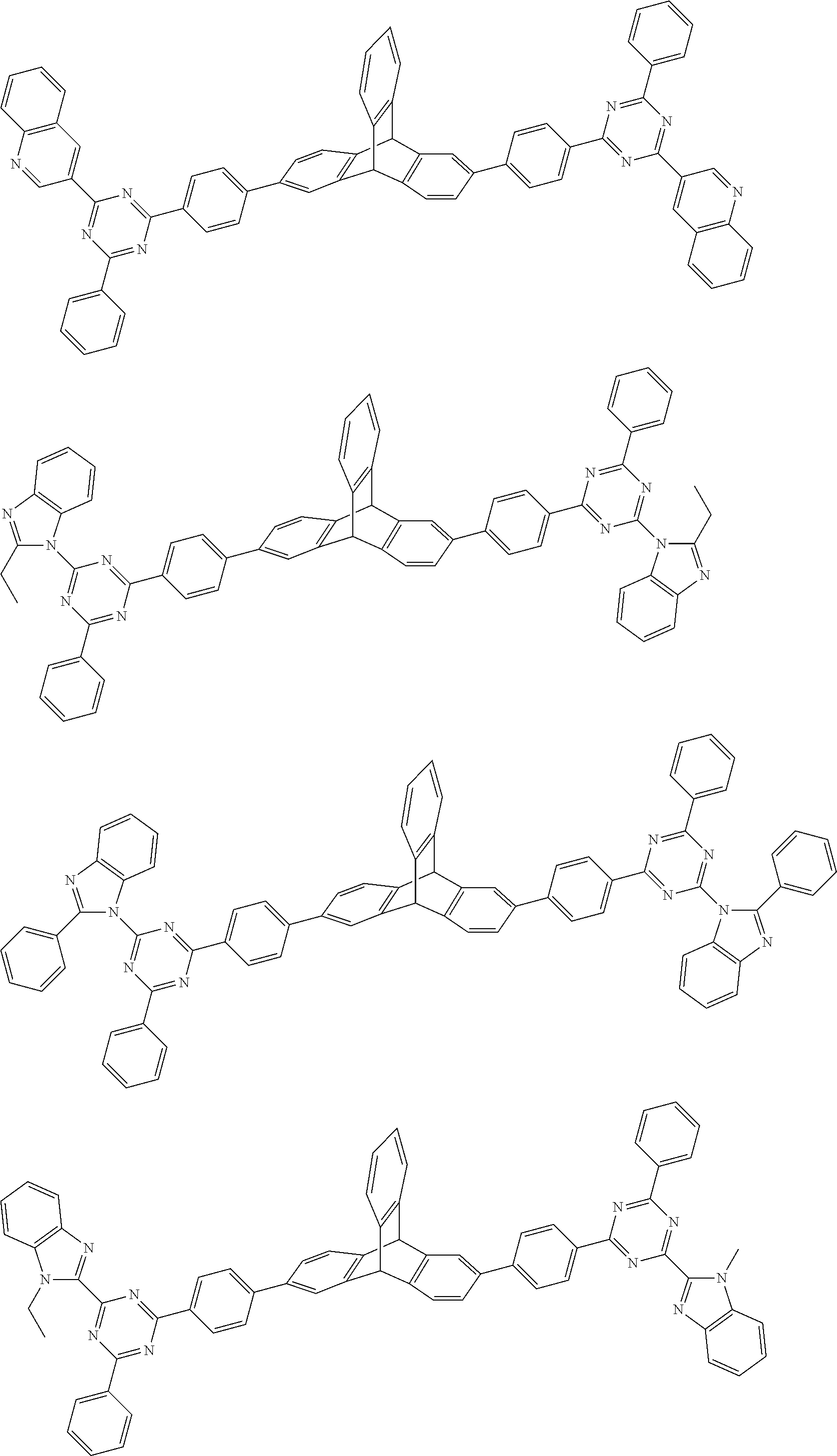

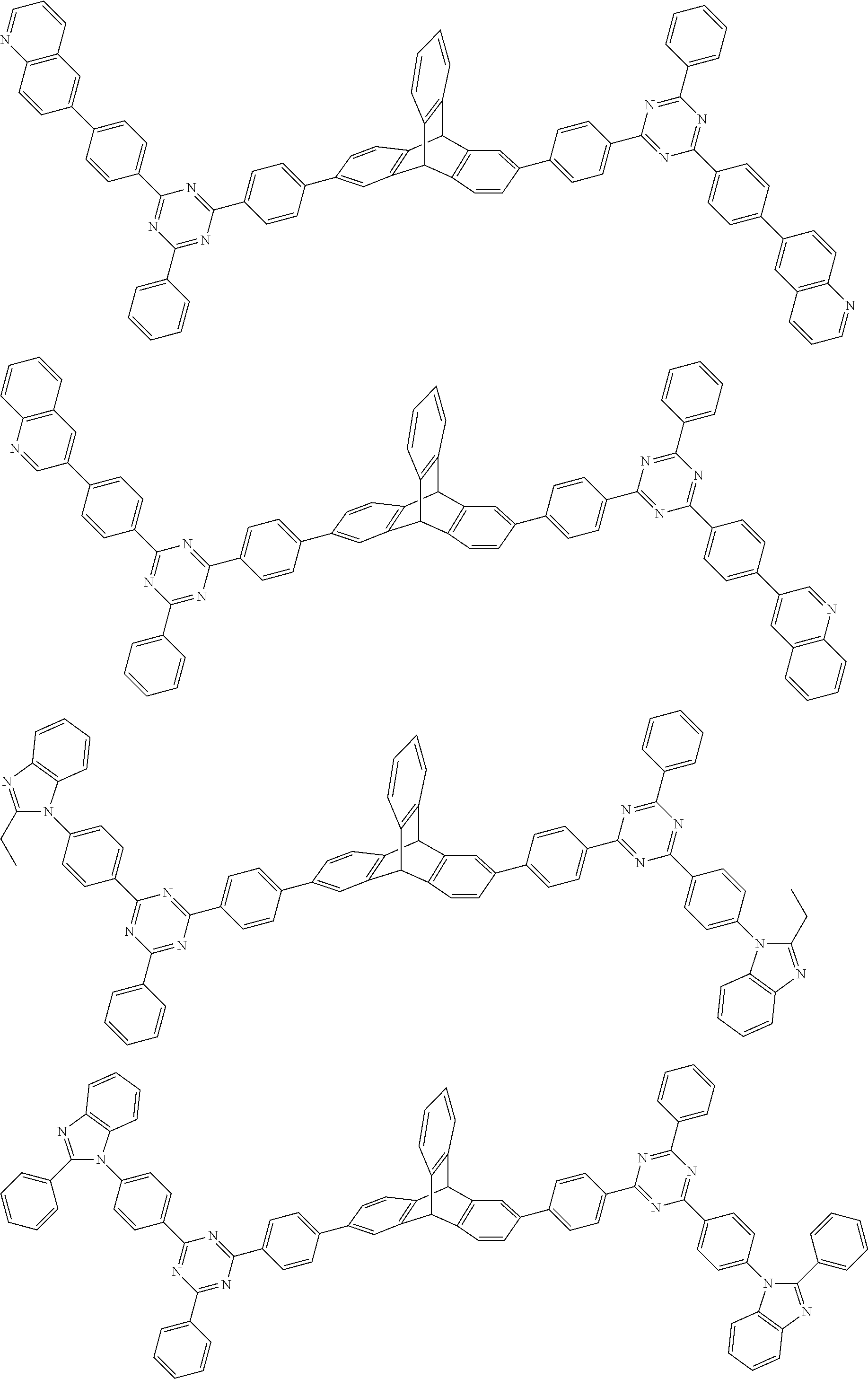

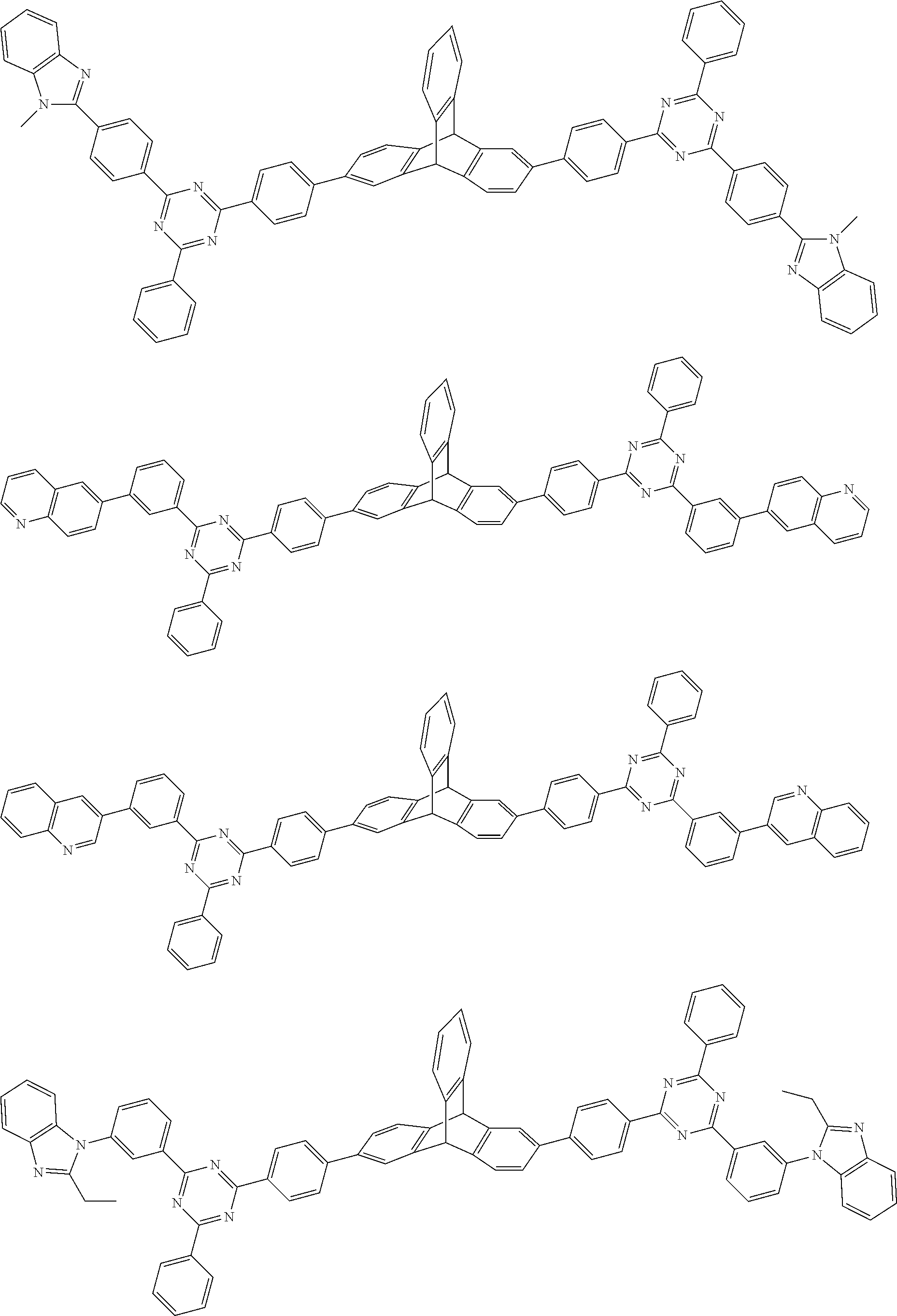

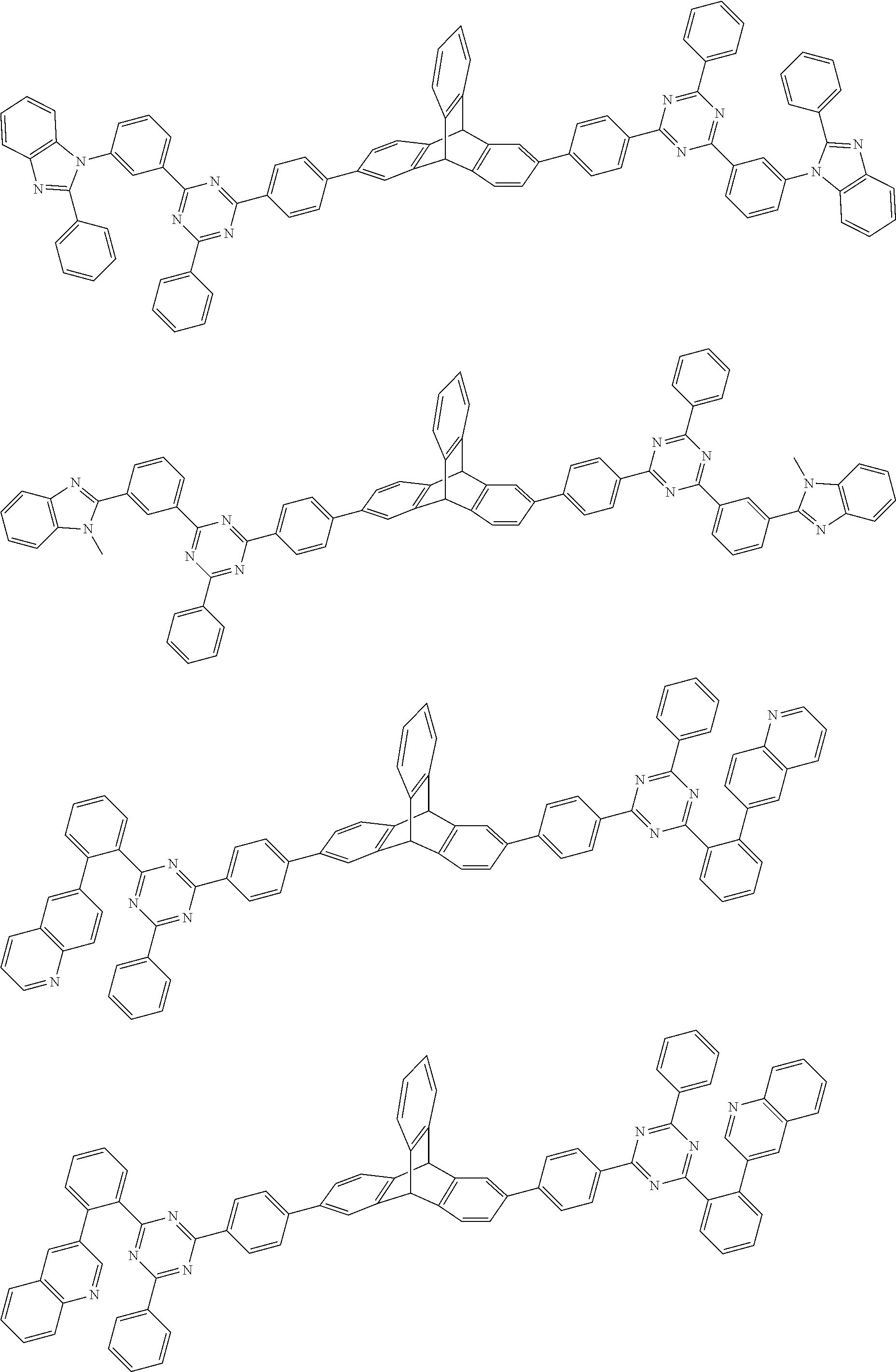

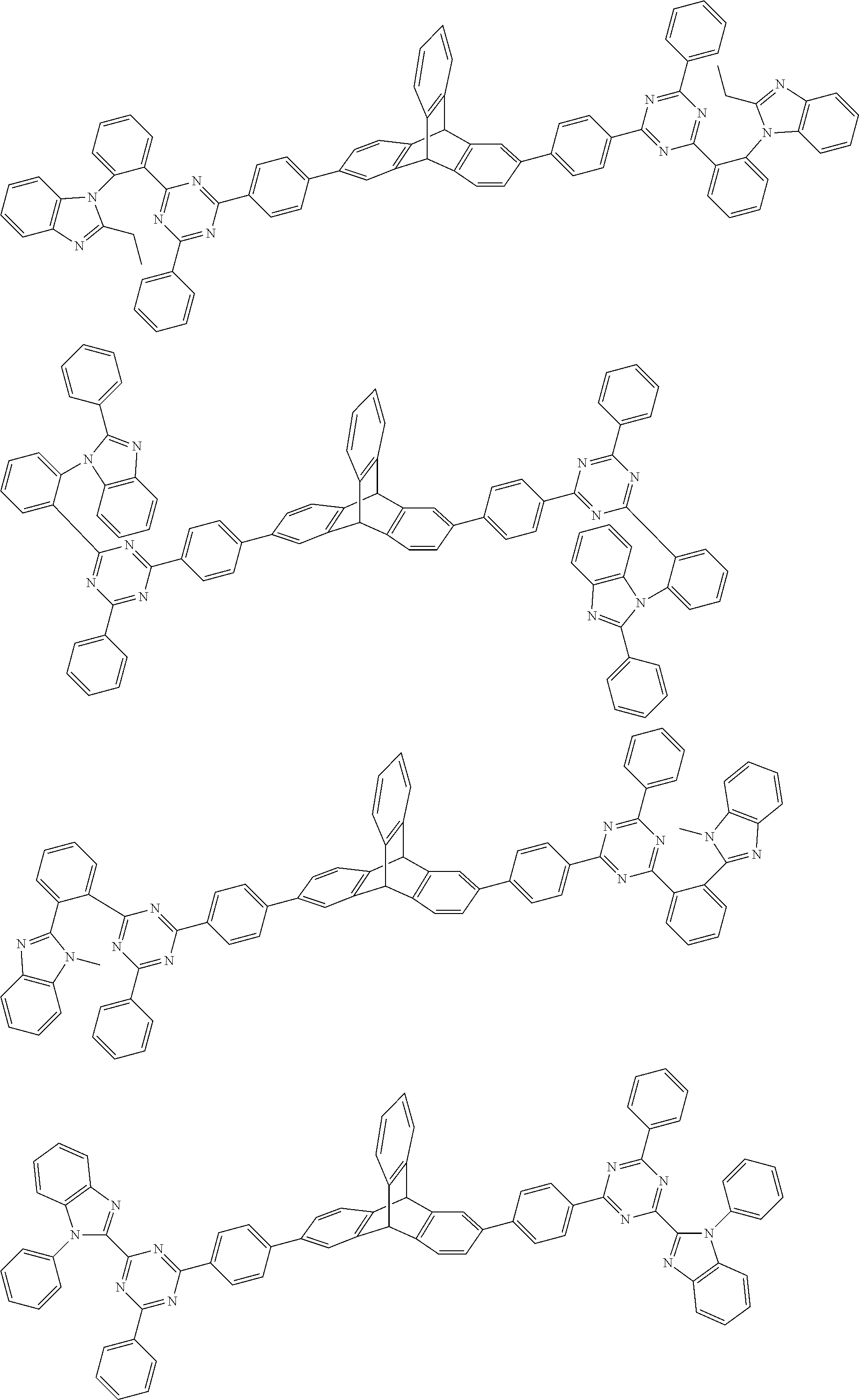

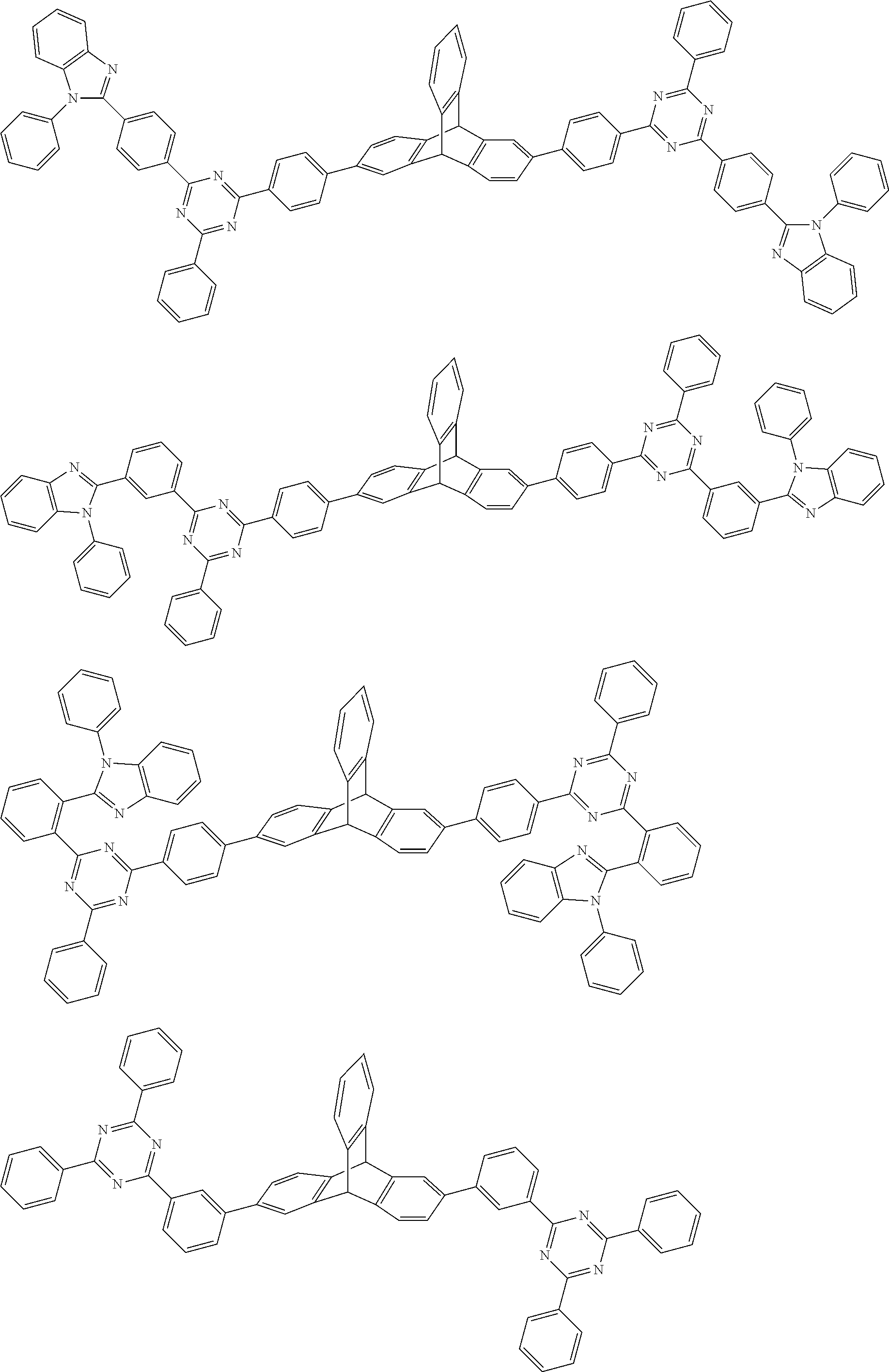

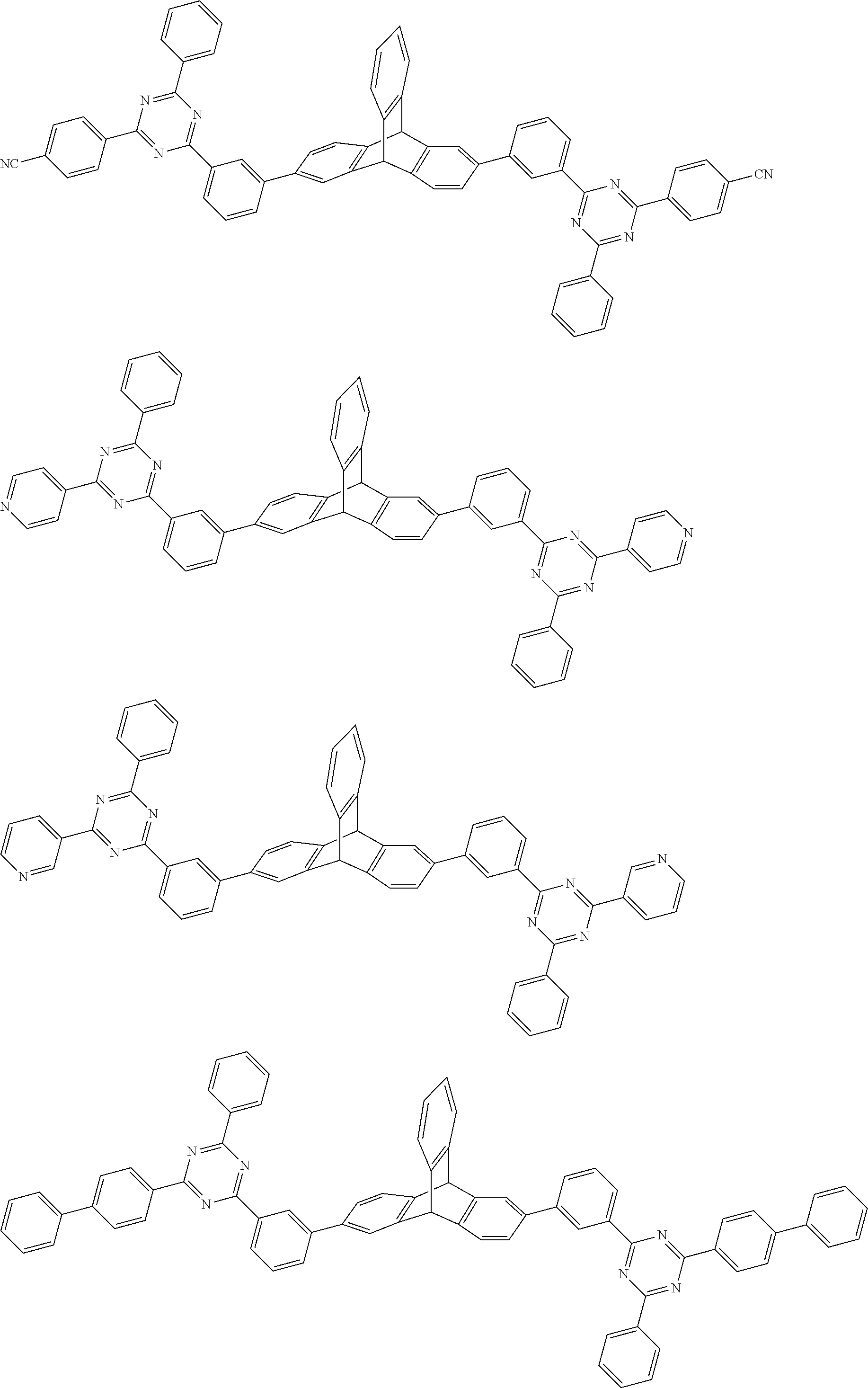

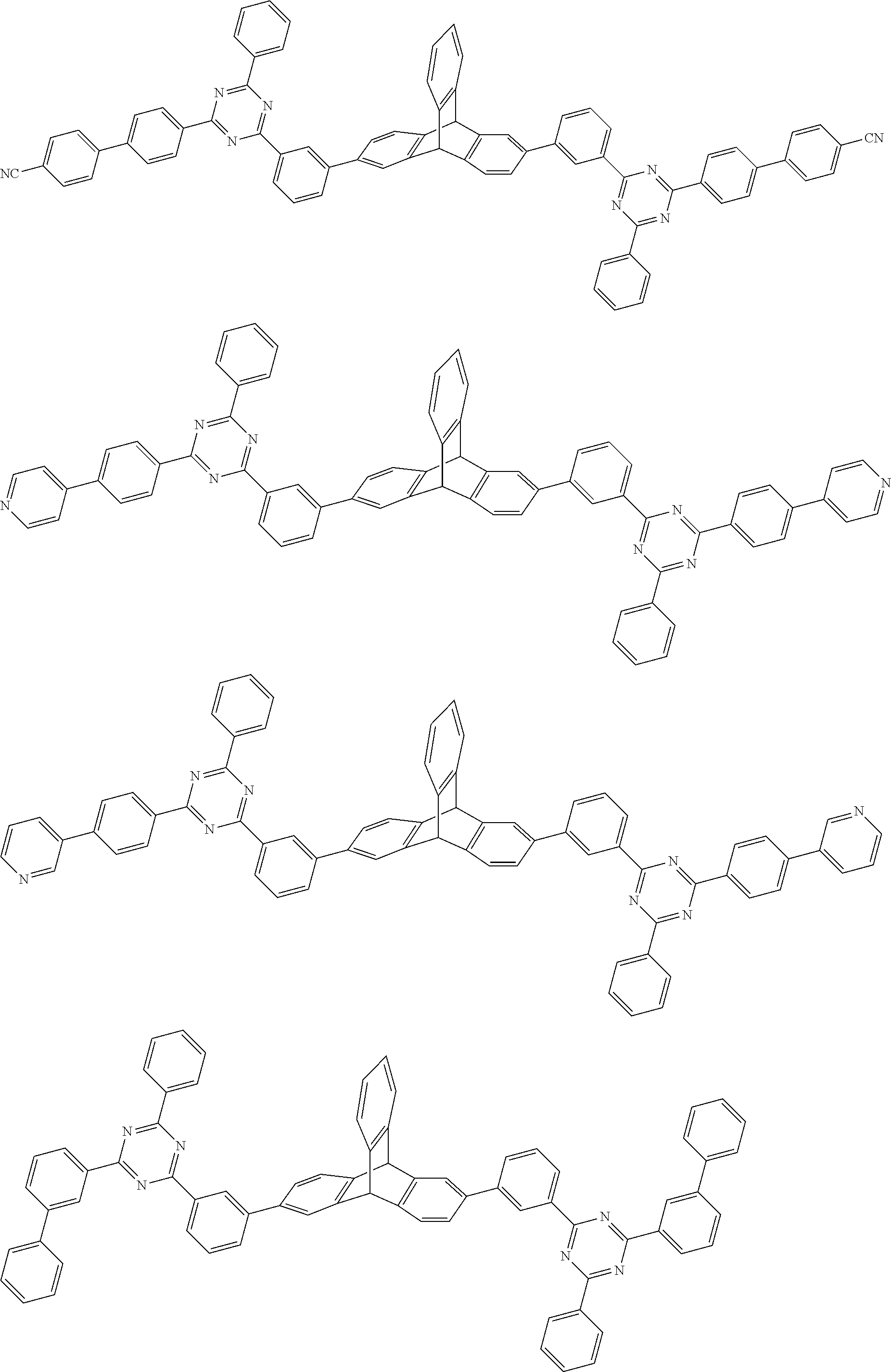

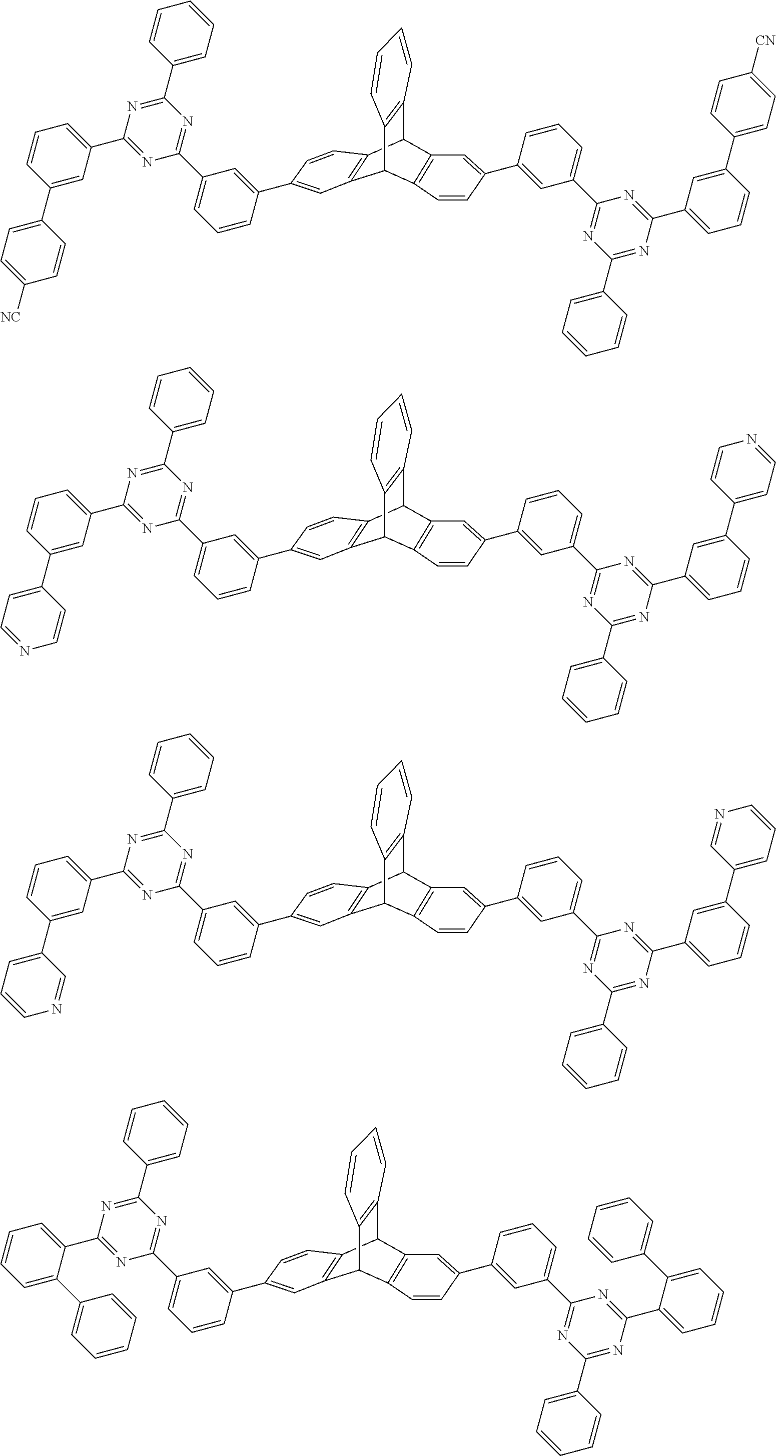

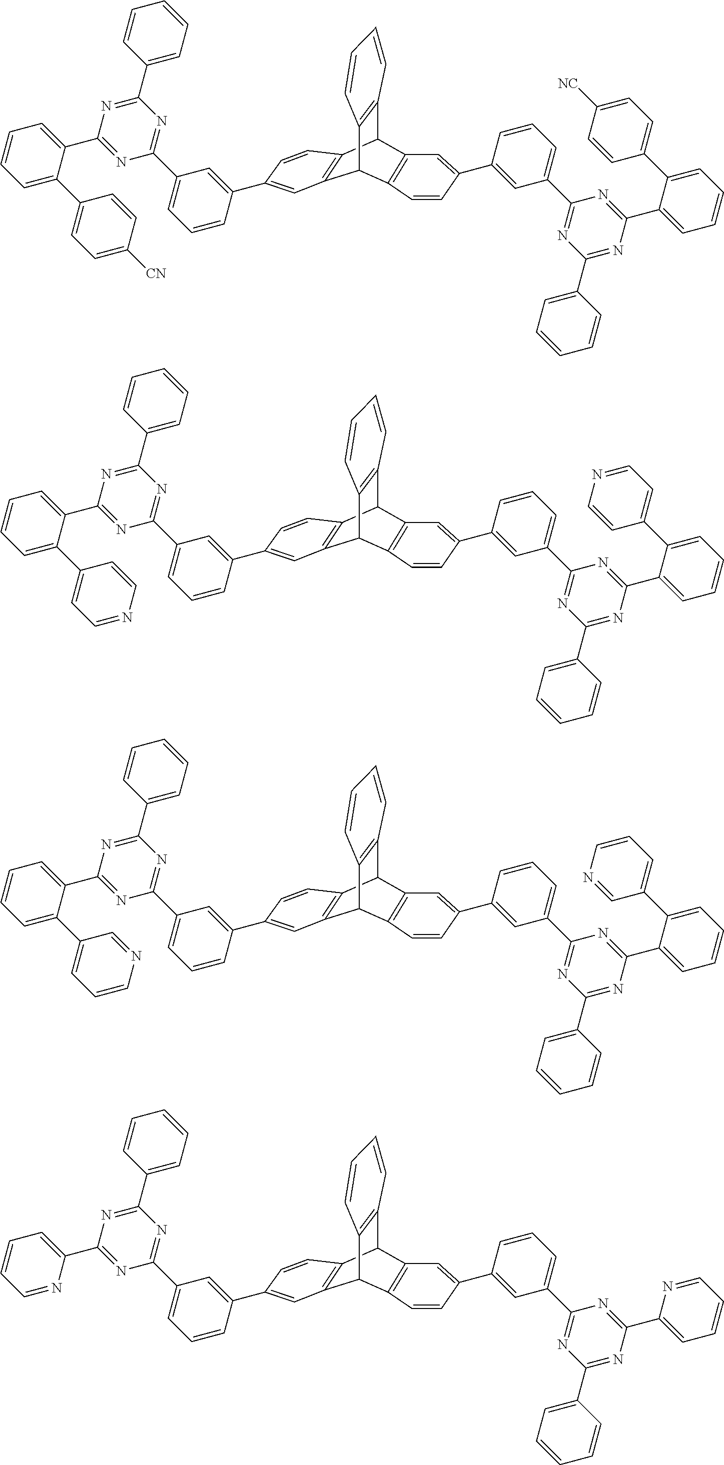

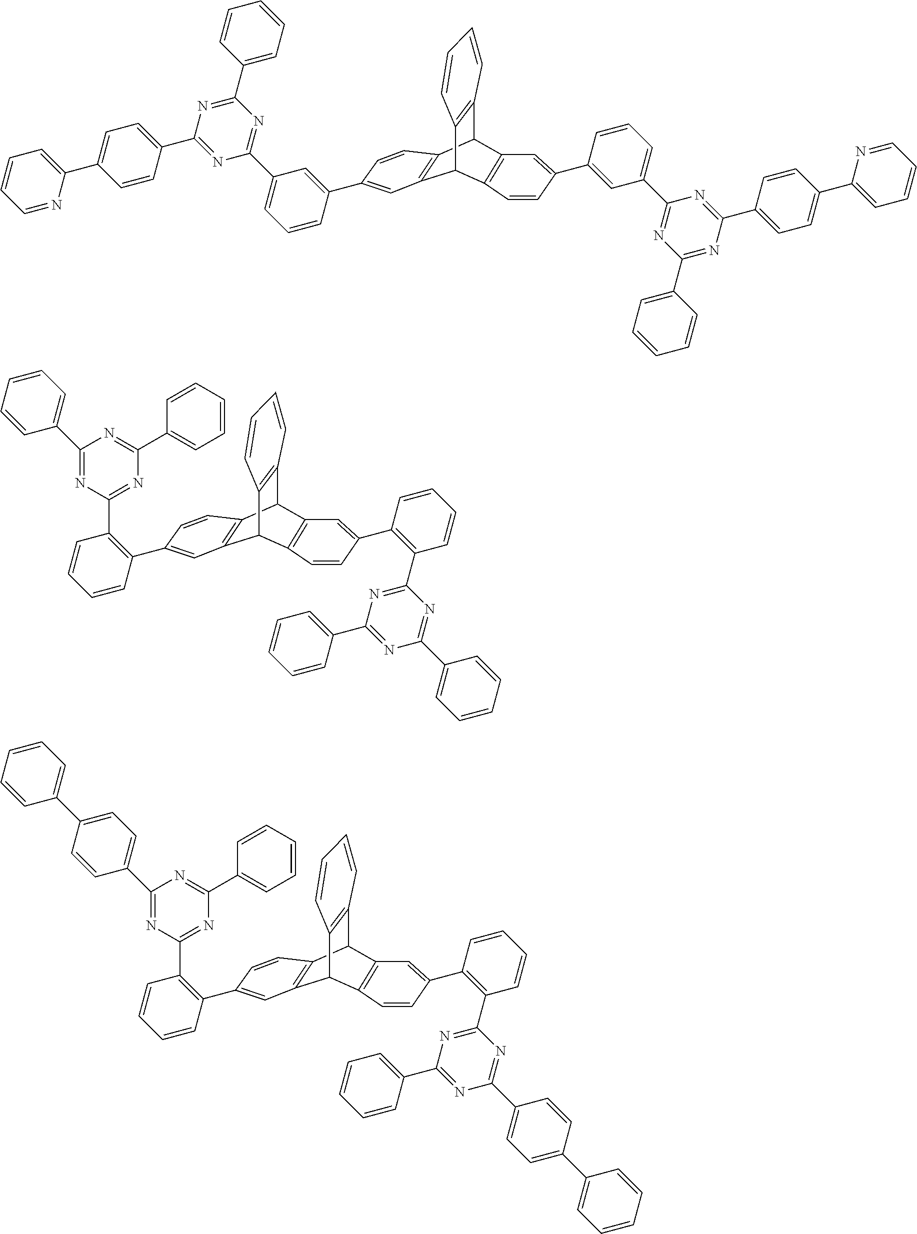 [SMILES: c1ccc(-c2nc(-c3ccc(-c4ccccn4)cc3)nc(-c3cccc(-c4ccc5c(c4)C4c6ccccc6C5c5cc(-c6cccc(-c7nc(-c8ccccc8)nc(-c8ccc(-c9ccccn9)cc8)n7)c6)ccc54)c3)n2)cc1 ; c1ccc(-c2nc(-c3ccccc3)nc(-c3ccccc3-c3ccc4c(c3)C3c5ccccc5C4c4cc(-c5ccccc5-c5nc(-c6ccccc6)nc(-c6ccccc6)n5)ccc43)n2)cc1 ; c1ccc(-c2ccc(-c3nc(-c4ccccc4)nc(-c4ccccc4-c4ccc5c(c4)C4c6ccccc6C5c5cc(-c6ccccc6-c6nc(-c7ccccc7)nc(-c7ccc(-c8ccccc8)cc7)n6)ccc54)n3)cc2)cc1]

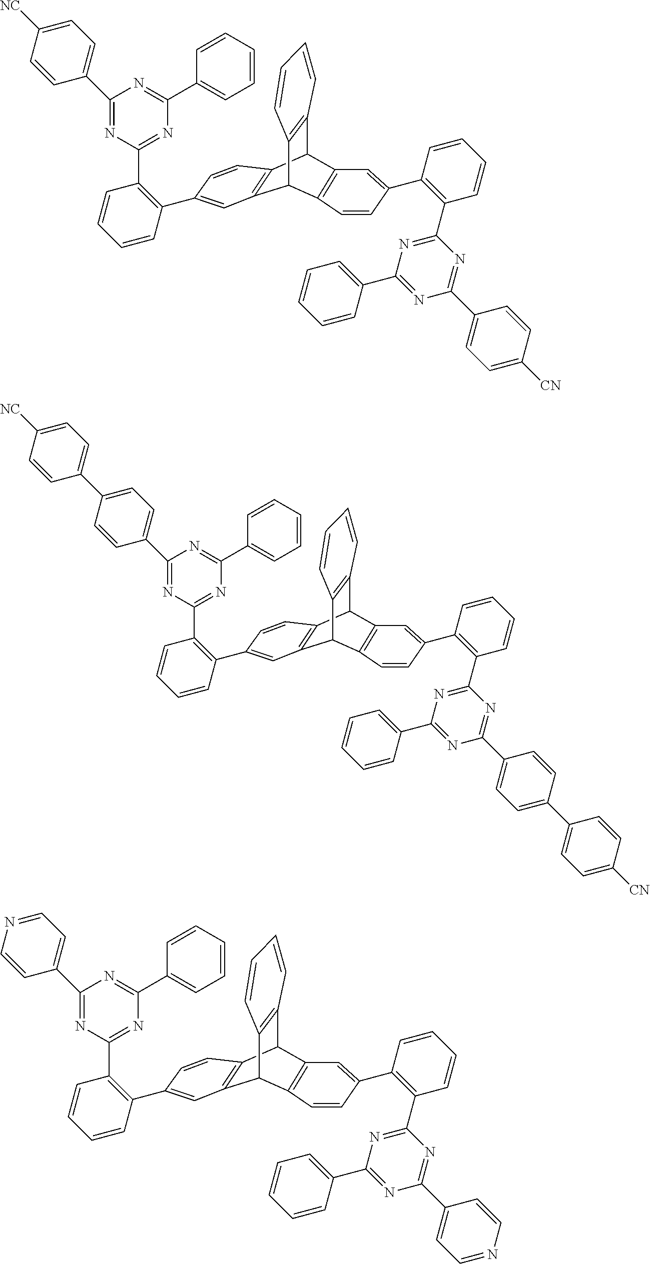 [SMILES: N#Cc1ccc(-c2nc(-c3ccccc3)nc(-c3ccccc3-c3ccc4c(c3)C3c5ccccc5C4c4cc(-c5ccccc5-c5nc(-c6ccccc6)nc(-c6ccc(C#N)cc6)n5)ccc43)n2)cc1 ; N#Cc1ccc(-c2ccc(-c3nc(-c4ccccc4)nc(-c4ccccc4-c4ccc5c(c4)C4c6ccccc6C5c5cc(-c6ccccc6-c6nc(-c7ccccc7)nc(-c7ccc(-c8ccc(C#N)cc8)cc7)n6)ccc54)n3)cc2)cc1 ; c1ccc(-c2nc(-c3ccncc3)nc(-c3ccccc3-c3ccc4c(c3)C3c5ccccc5C4c4cc(-c5ccccc5-c5nc(-c6ccccc6)nc(-c6ccncc6)n5)ccc43)n2)cc1]

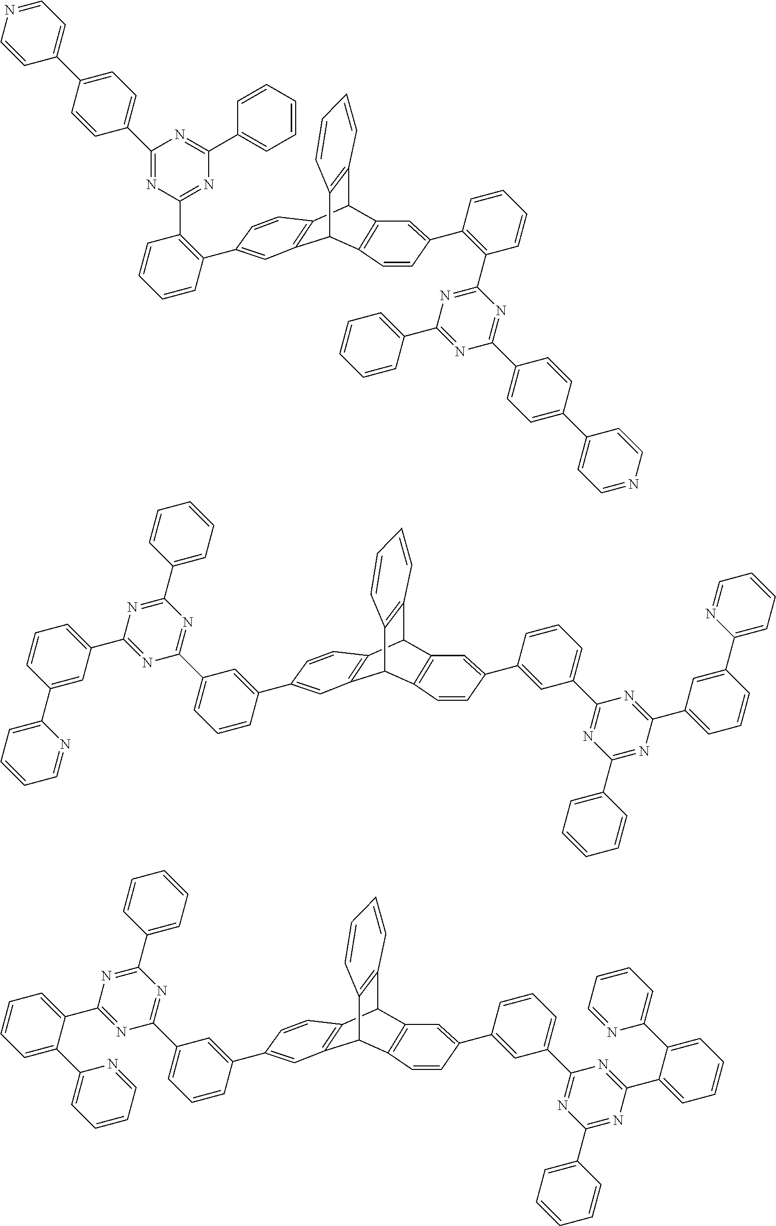 [SMILES: c1ccc(-c2nc(-c3ccc(-c4ccncc4)cc3)nc(-c3ccccc3-c3ccc4c(c3)C3c5ccccc5C4c4cc(-c5ccccc5-c5nc(-c6ccccc6)nc(-c6ccc(-c7ccncc7)cc6)n5)ccc43)n2)cc1 ; c1ccc(-c2nc(-c3cccc(-c4ccc5c(c4)C4c6ccccc6C5c5cc(-c6cccc(-c7nc(-c8ccccc8)nc(-c8cccc(-c9ccccn9)c8)n7)c6)ccc54)c3)nc(-c3cccc(-c4ccccn4)c3)n2)cc1 ; c1ccc(-c2nc(-c3cccc(-c4ccc5c(c4)C4c6ccccc6C5c5cc(-c6cccc(-c7nc(-c8ccccc8)nc(-c8ccccc8-c8ccccn8)n7)c6)ccc54)c3)nc(-c3ccccc3-c3ccccn3)n2)cc1]

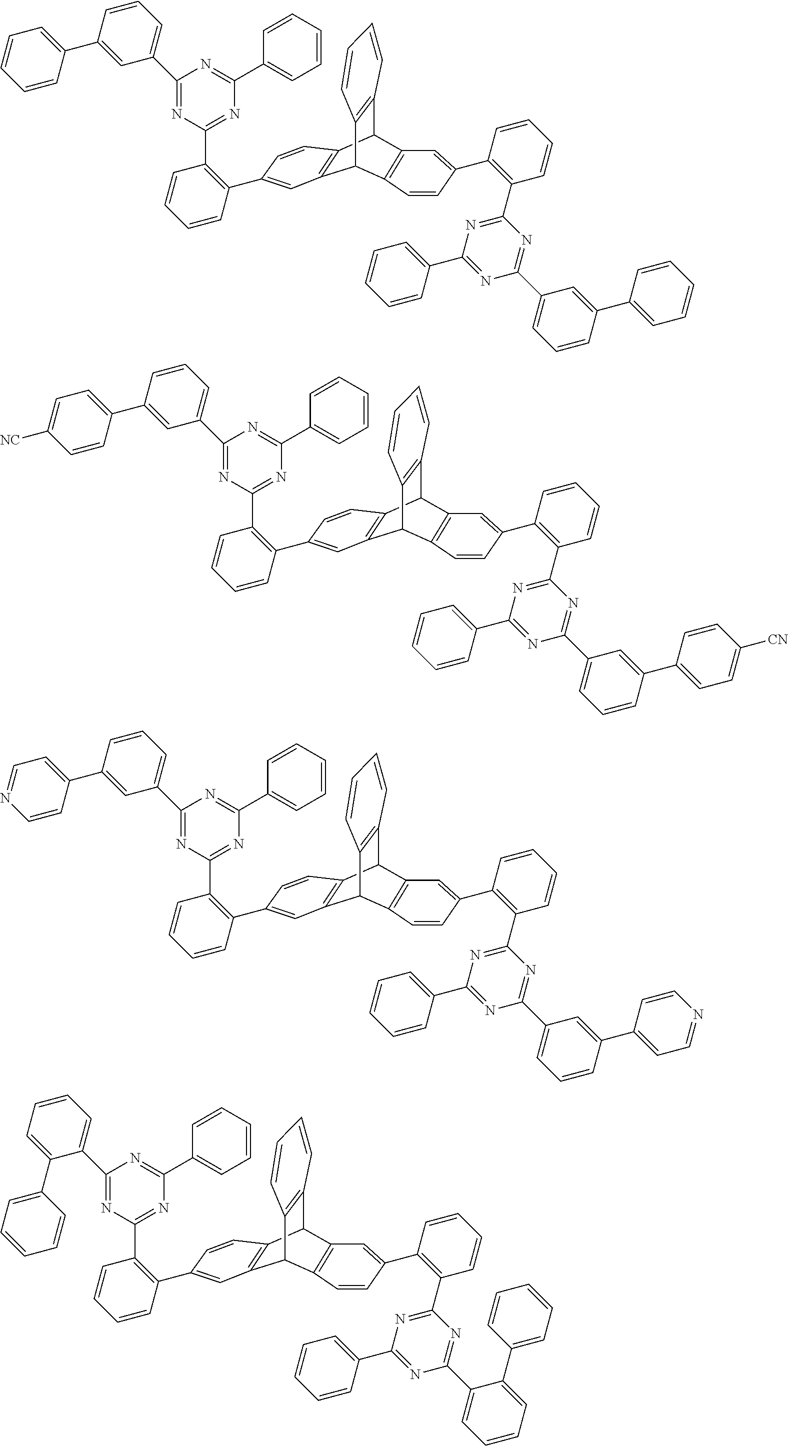

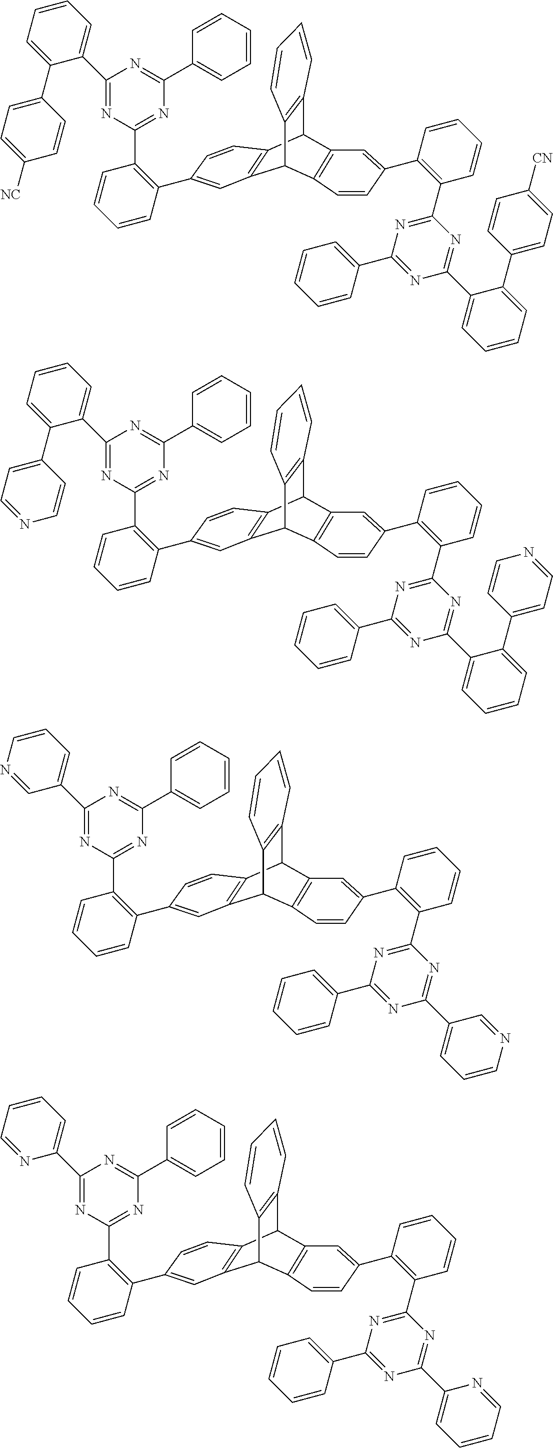

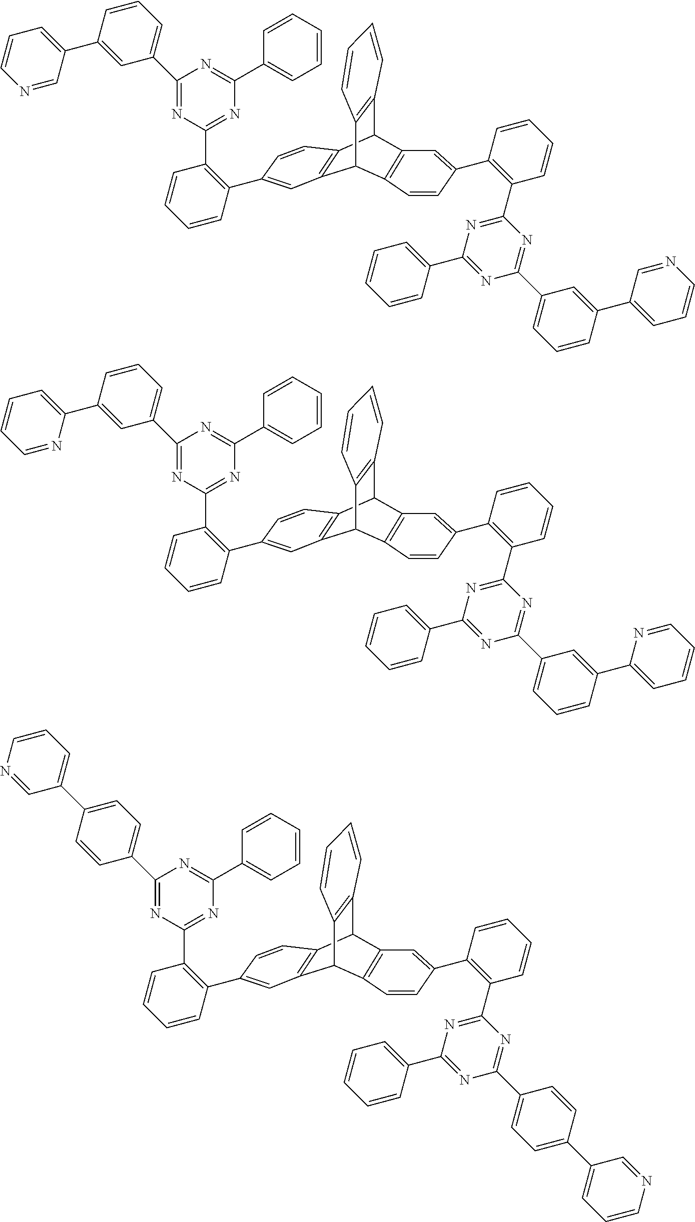 [SMILES: c1ccc(-c2nc(-c3cccc(-c4cccnc4)c3)nc(-c3ccccc3-c3ccc4c(c3)C3c5ccccc5C4c4cc(-c5ccccc5-c5nc(-c6ccccc6)nc(-c6cccc(-c7cccnc7)c6)n5)ccc43)n2)cc1 ; c1ccc(-c2nc(-c3cccc(-c4ccccn4)c3)nc(-c3ccccc3-c3ccc4c(c3)C3c5ccccc5C4c4cc(-c5ccccc5-c5nc(-c6ccccc6)nc(-c6cccc(-c7ccccn7)c6)n5)ccc43)n2)cc1 ; c1ccc(-c2nc(-c3ccc(-c4cccnc4)cc3)nc(-c3ccccc3-c3ccc4c(c3)C3c5ccccc5C4c4cc(-c5ccccc5-c5nc(-c6ccccc6)nc(-c6ccc(-c7cccnc7)cc6)n5)ccc43)n2)cc1]

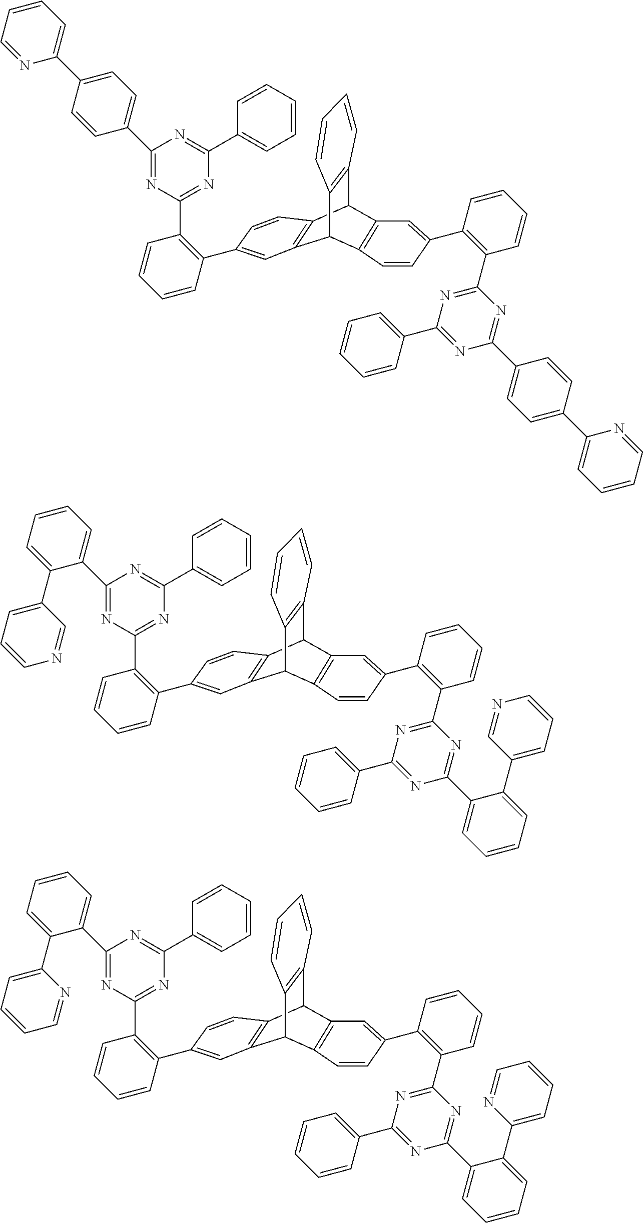 [SMILES: c1ccc(-c2nc(-c3ccc(-c4ccccn4)cc3)nc(-c3ccccc3-c3ccc4c(c3)C3c5ccccc5C4c4cc(-c5ccccc5-c5nc(-c6ccccc6)nc(-c6ccc(-c7ccccn7)cc6)n5)ccc43)n2)cc1 ; c1ccc(-c2nc(-c3ccccc3-c3cccnc3)nc(-c3ccccc3-c3ccc4c(c3)C3c5ccccc5C4c4cc(-c5ccccc5-c5nc(-c6ccccc6)nc(-c6ccccc6-c6cccnc6)n5)ccc43)n2)cc1 ; c1ccc(-c2nc(-c3ccccc3-c3ccc4c(c3)C3c5ccccc5C4c4cc(-c5ccccc5-c5nc(-c6ccccc6)nc(-c6ccccc6-c6ccccn6)n5)ccc43)nc(-c3ccccc3-c3ccccn3)n2)cc1]

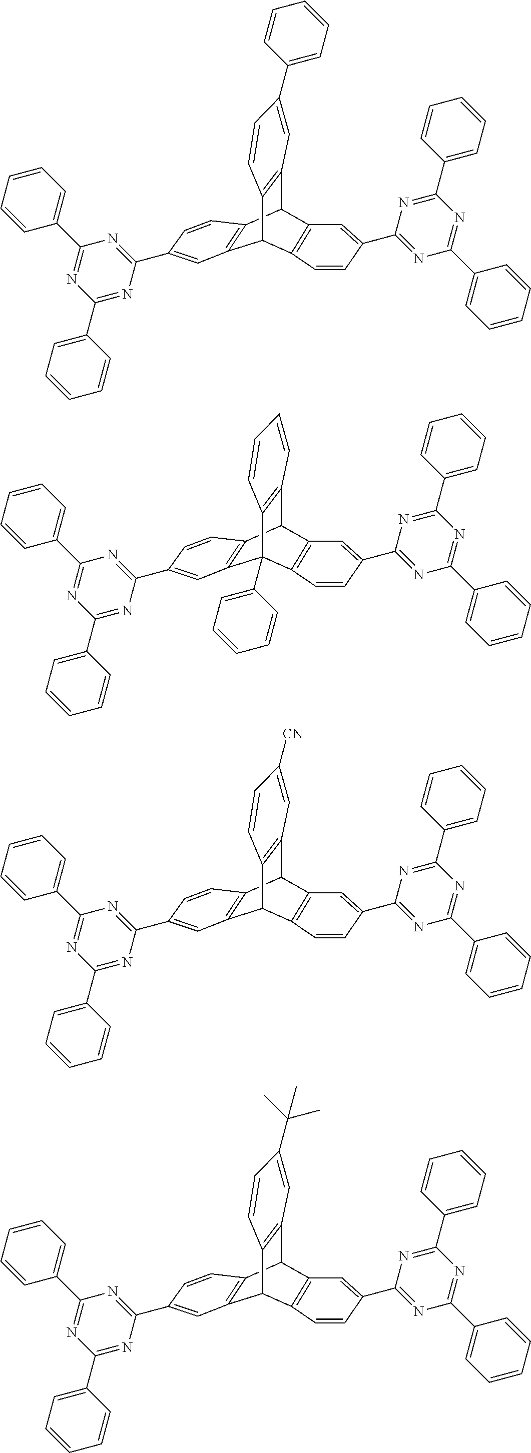 [SMILES: c1ccc(-c2ccc3c(c2)C2c4ccc(-c5nc(-c6ccccc6)nc(-c6ccccc6)n5)cc4C3c3ccc(-c4nc(-c5ccccc5)nc(-c5ccccc5)n4)cc32)cc1 ; c1ccc(-c2nc(-c3ccccc3)nc(-c3ccc4c(c3)C3c5ccccc5C4(c4ccccc4)c4cc(-c5nc(-c6ccccc6)nc(-c6ccccc6)n5)ccc43)n2)cc1 ; N#Cc1ccc2c(c1)C1c3ccc(-c4nc(-c5ccccc5)nc(-c5ccccc5)n4)cc3C2c2ccc(-c3nc(-c4ccccc4)nc(-c4ccccc4)n3)cc21 ; CC(C)(C)c1ccc2c(c1)C1c3ccc(-c4nc(-c5ccccc5)nc(-c5ccccc5)n4)cc3C2c2ccc(-c3nc(-c4ccccc4)nc(-c4ccccc4)n3)cc21]

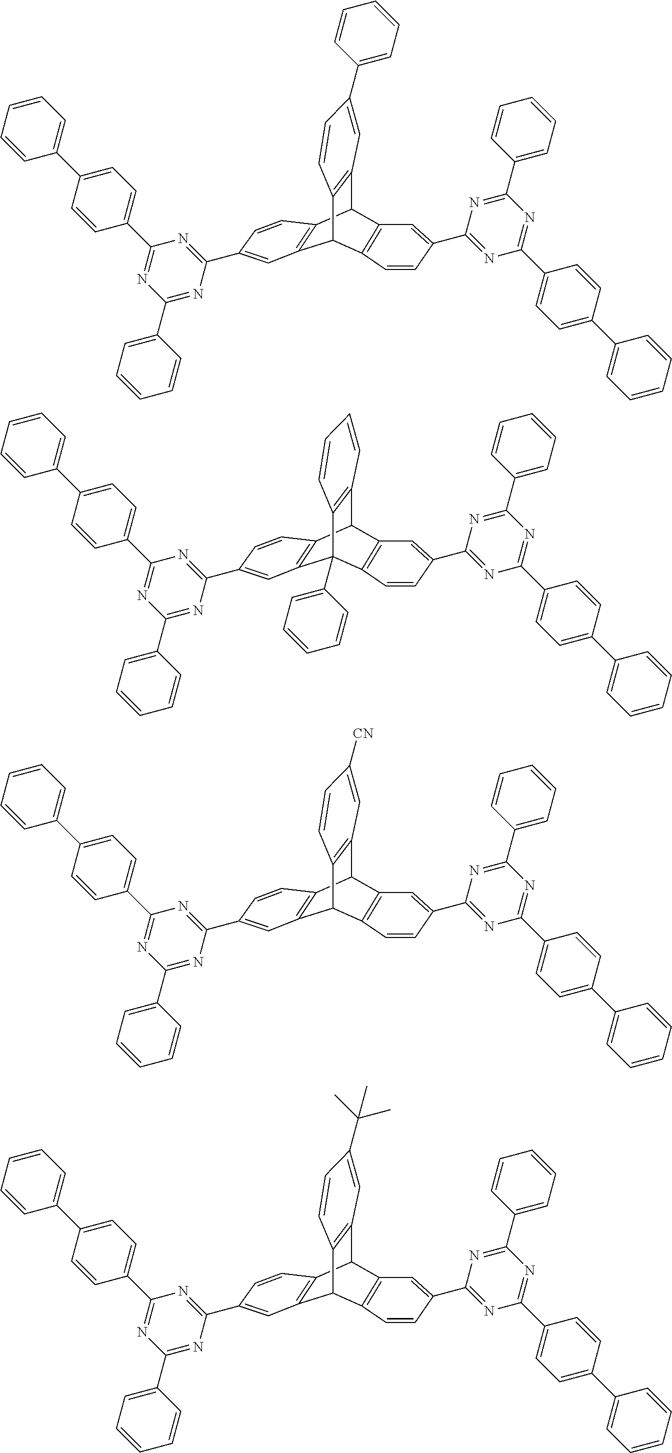

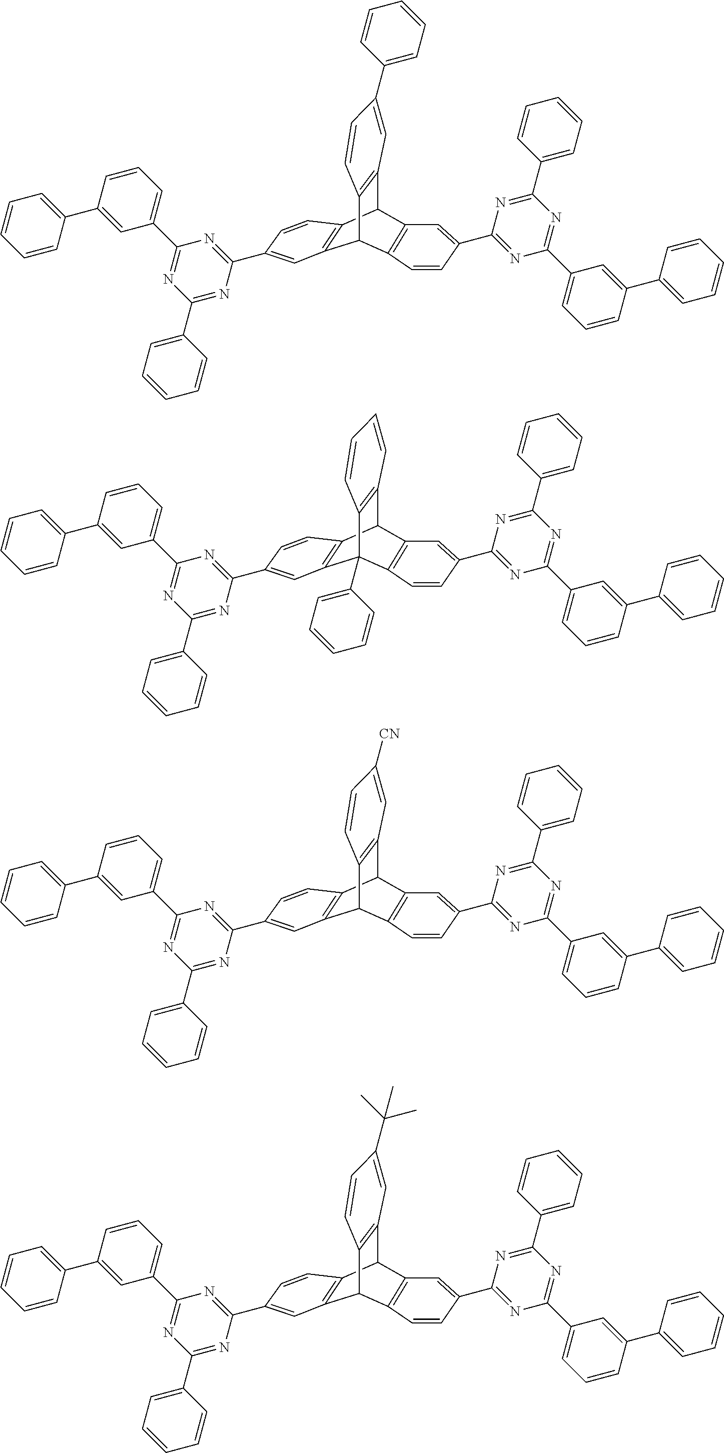

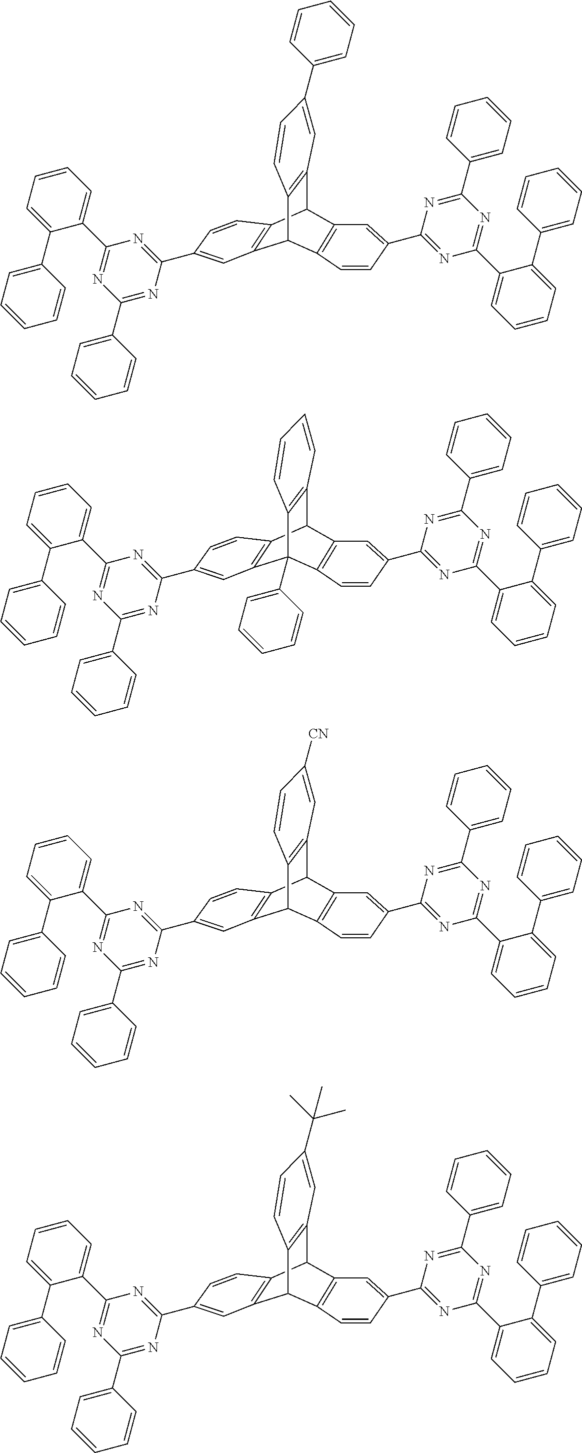

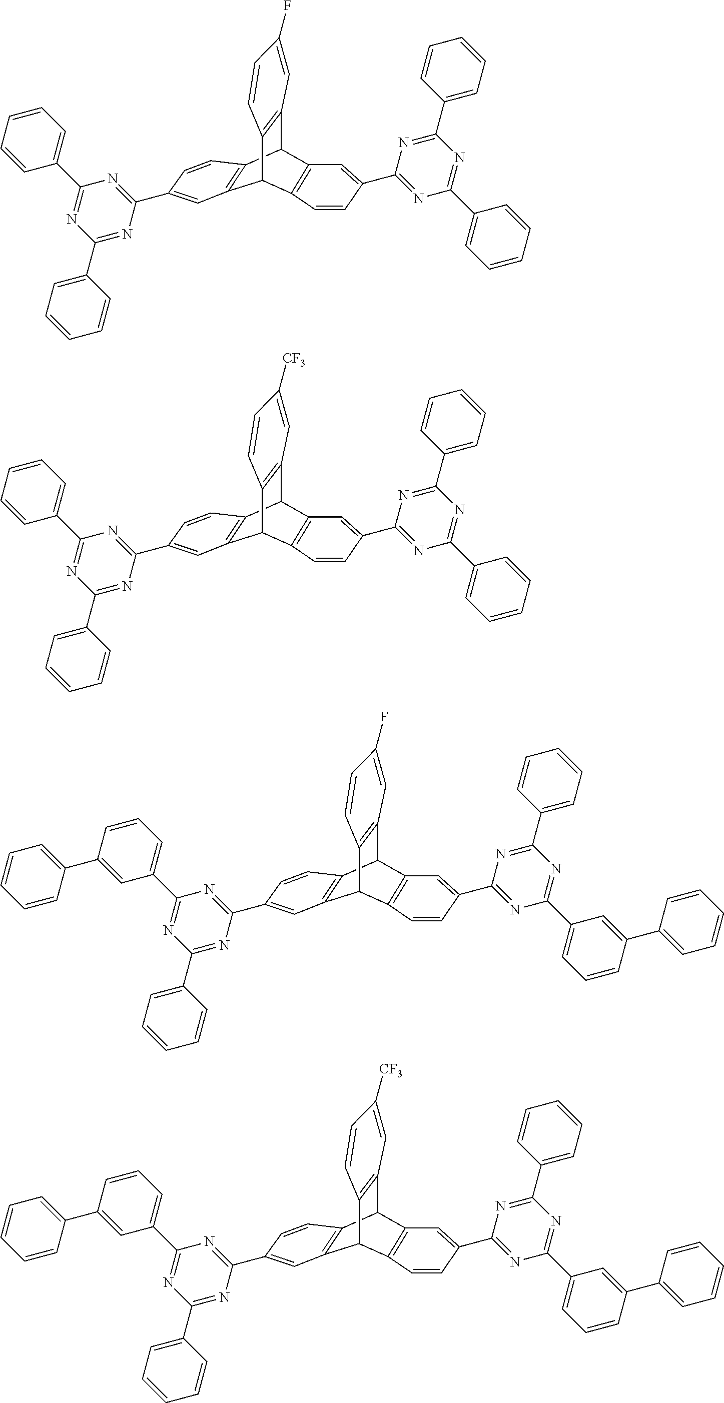 [SMILES: Fc1ccc2c(c1)C1c3ccc(-c4nc(-c5ccccc5)nc(-c5ccccc5)n4)cc3C2c2ccc(-c3nc(-c4ccccc4)nc(-c4ccccc4)n3)cc21 ; FC(F)(F)c1ccc2c(c1)C1c3ccc(-c4nc(-c5ccccc5)nc(-c5ccccc5)n4)cc3C2c2ccc(-c3nc(-c4ccccc4)nc(-c4ccccc4)n3)cc21 ; Fc1ccc2c(c1)C1c3ccc(-c4nc(-c5ccccc5)nc(-c5cccc(-c6ccccc6)c5)n4)cc3C2c2ccc(-c3nc(-c4ccccc4)nc(-c4cccc(-c5ccccc5)c4)n3)cc21 ; FC(F)(F)c1ccc2c(c1)C1c3ccc(-c4nc(-c5ccccc5)nc(-c5cccc(-c6ccccc6)c5)n4)cc3C2c2ccc(-c3nc(-c4ccccc4)nc(-c4cccc(-c5ccccc5)c4)n3)cc21]

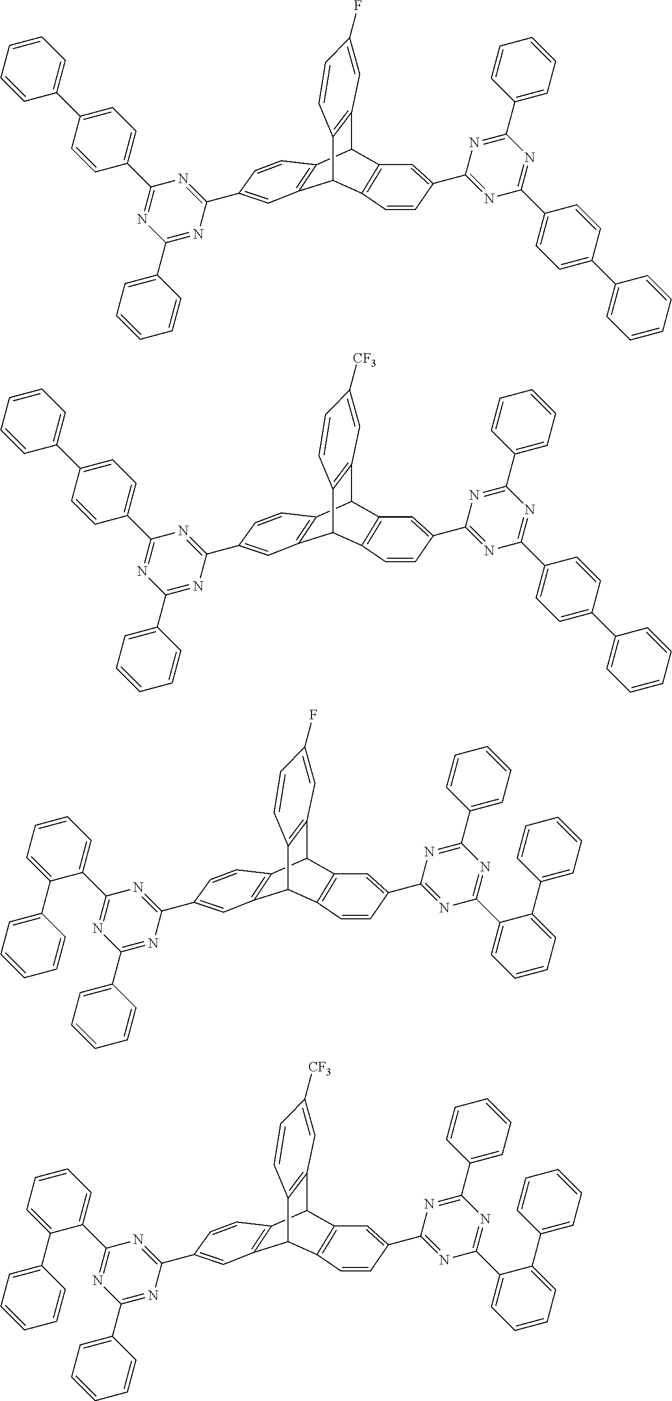 [SMILES: Fc1ccc2c(c1)C1c3ccc(-c4nc(-c5ccccc5)nc(-c5ccc(-c6ccccc6)cc5)n4)cc3C2c2ccc(-c3nc(-c4ccccc4)nc(-c4ccc(-c5ccccc5)cc4)n3)cc21 ; FC(F)(F)c1ccc2c(c1)C1c3ccc(-c4nc(-c5ccccc5)nc(-c5ccc(-c6ccccc6)cc5)n4)cc3C2c2ccc(-c3nc(-c4ccccc4)nc(-c4ccc(-c5ccccc5)cc4)n3)cc21 ; Fc1ccc2c(c1)C1c3ccc(-c4nc(-c5ccccc5)nc(-c5ccccc5-c5ccccc5)n4)cc3C2c2ccc(-c3nc(-c4ccccc4)nc(-c4ccccc4-c4ccccc4)n3)cc21 ; FC(F)(F)c1ccc2c(c1)C1c3ccc(-c4nc(-c5ccccc5)nc(-c5ccccc5-c5ccccc5)n4)cc3C2c2ccc(-c3nc(-c4ccccc4)nc(-c4ccccc4-c4ccccc4)n3)cc21]

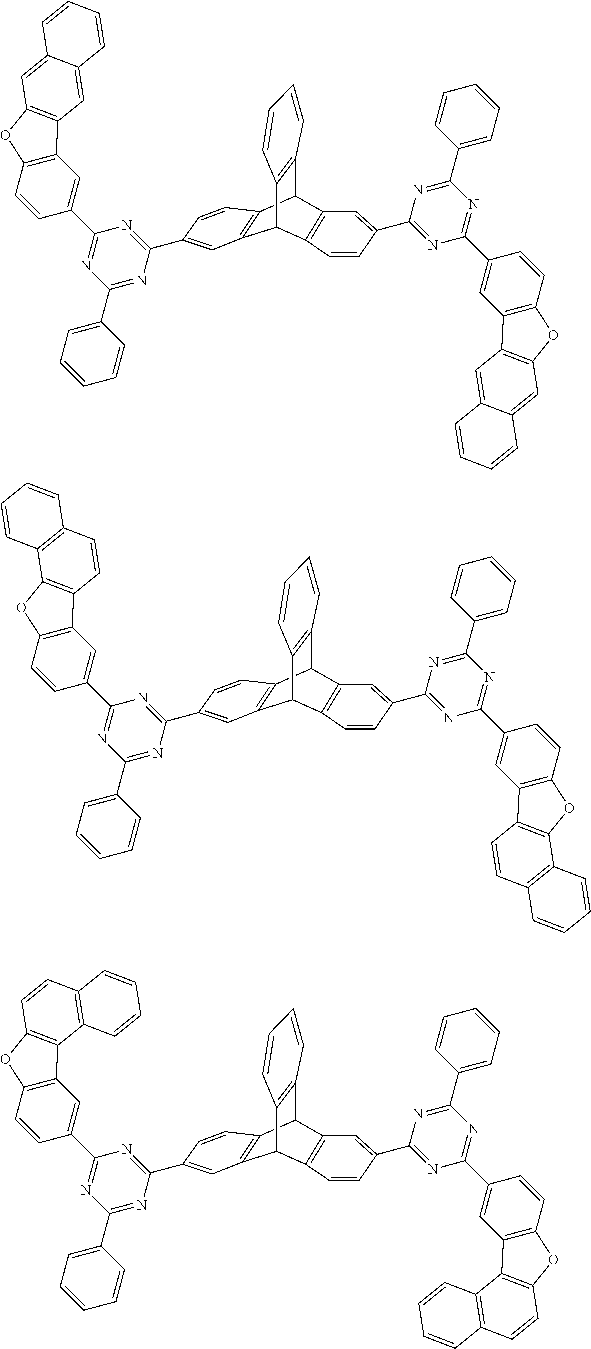 [SMILES: c1ccc(-c2nc(-c3ccc4c(c3)C3c5ccccc5C4c4cc(-c5nc(-c6ccccc6)nc(-c6ccc7oc8cc9ccccc9cc8c7c6)n5)ccc43)nc(-c3ccc4oc5cc6ccccc6cc5c4c3)n2)cc1 ; c1ccc(-c2nc(-c3ccc4c(c3)C3c5ccccc5C4c4cc(-c5nc(-c6ccccc6)nc(-c6ccc7oc8c9ccccc9ccc8c7c6)n5)ccc43)nc(-c3ccc4oc5c6ccccc6ccc5c4c3)n2)cc1 ; c1ccc(-c2nc(-c3ccc4c(c3)C3c5ccccc5C4c4cc(-c5nc(-c6ccccc6)nc(-c6ccc7oc8ccc9ccccc9c8c7c6)n5)ccc43)nc(-c3ccc4oc5ccc6ccccc6c5c4c3)n2)cc1]

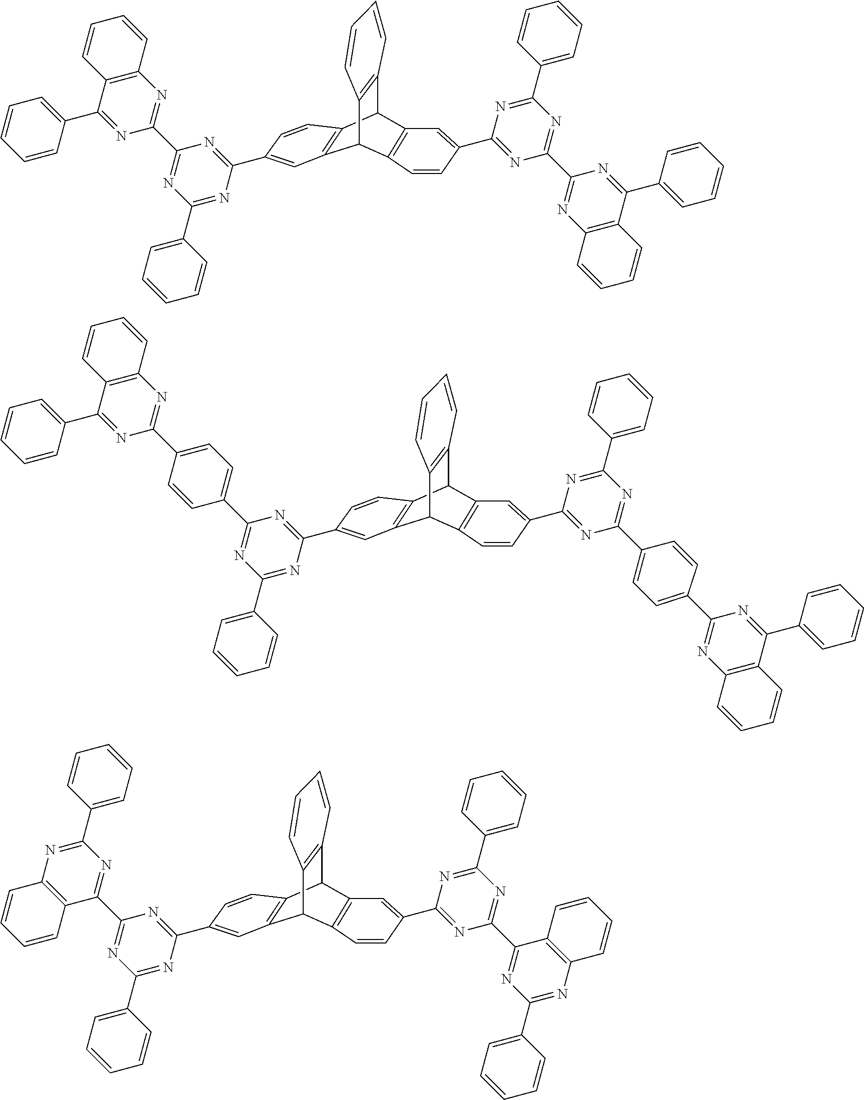

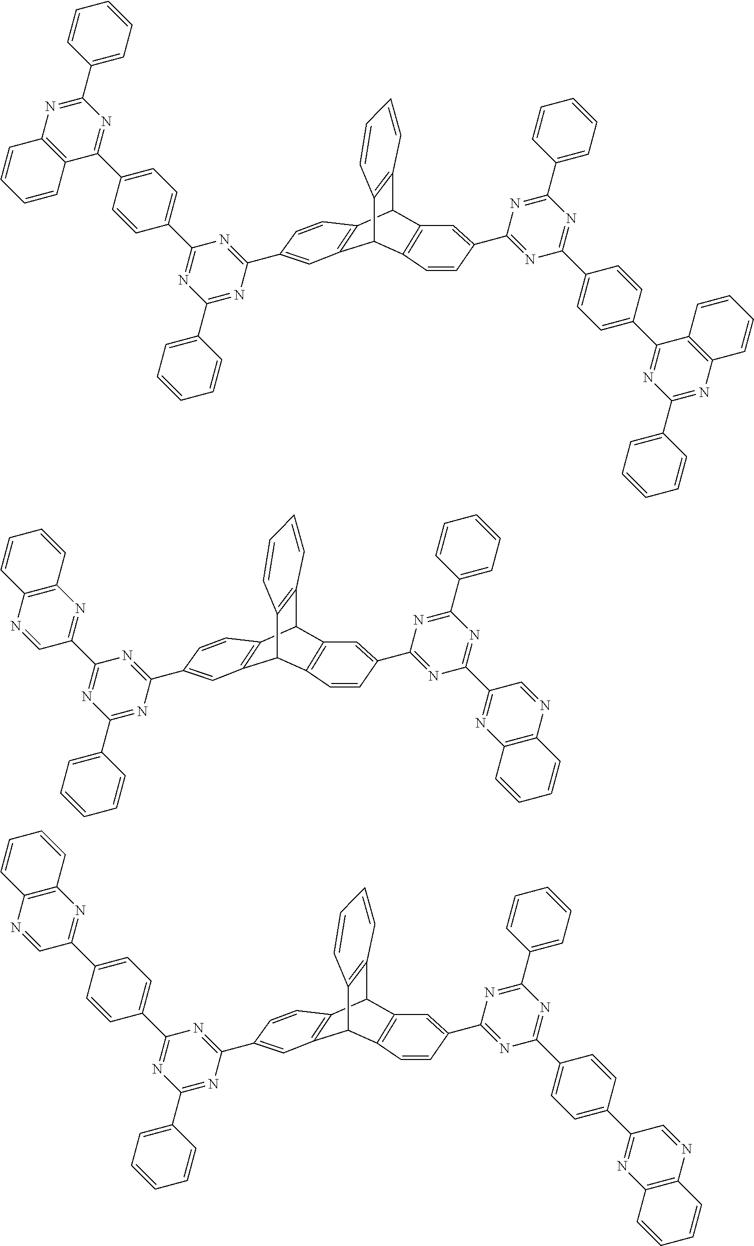

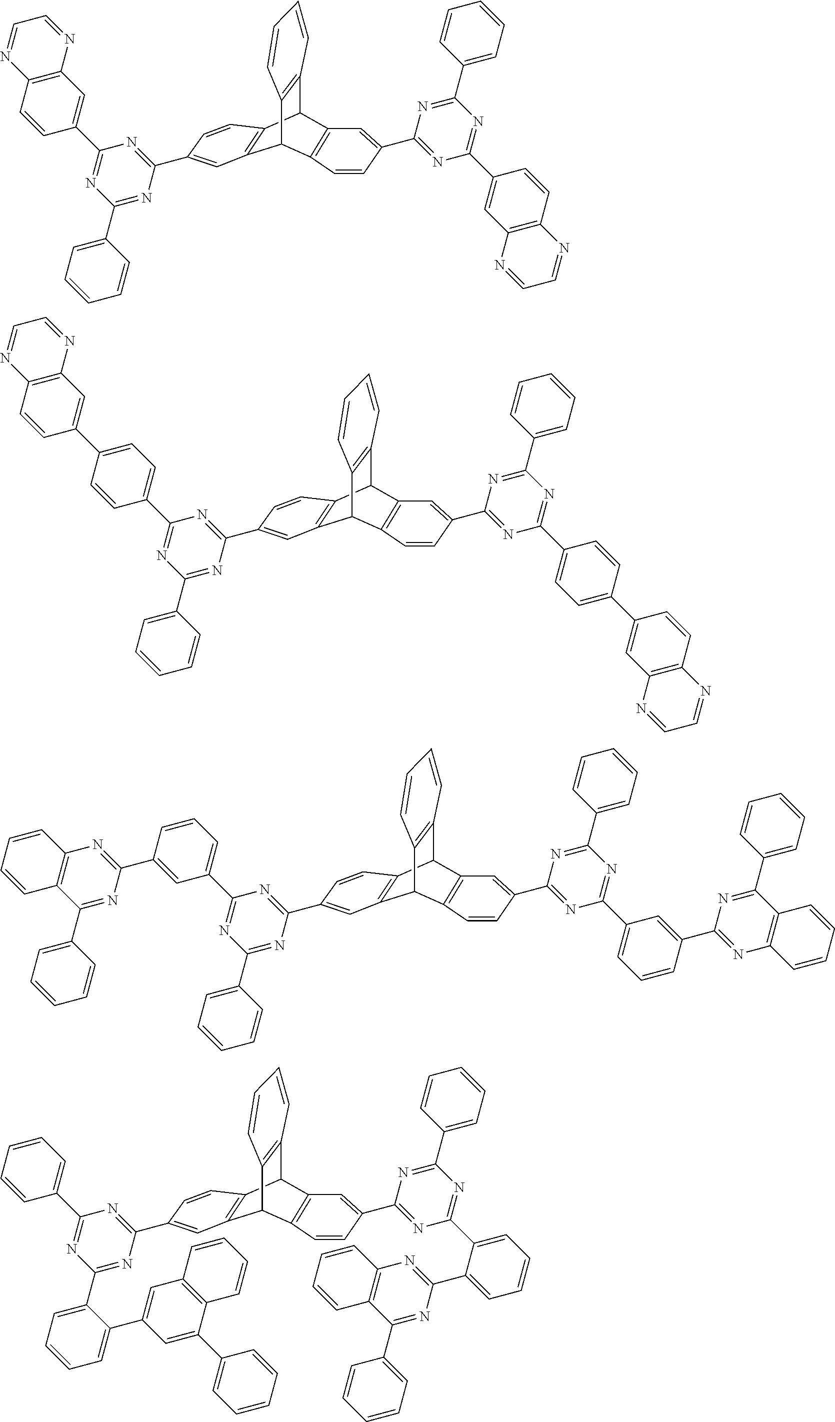

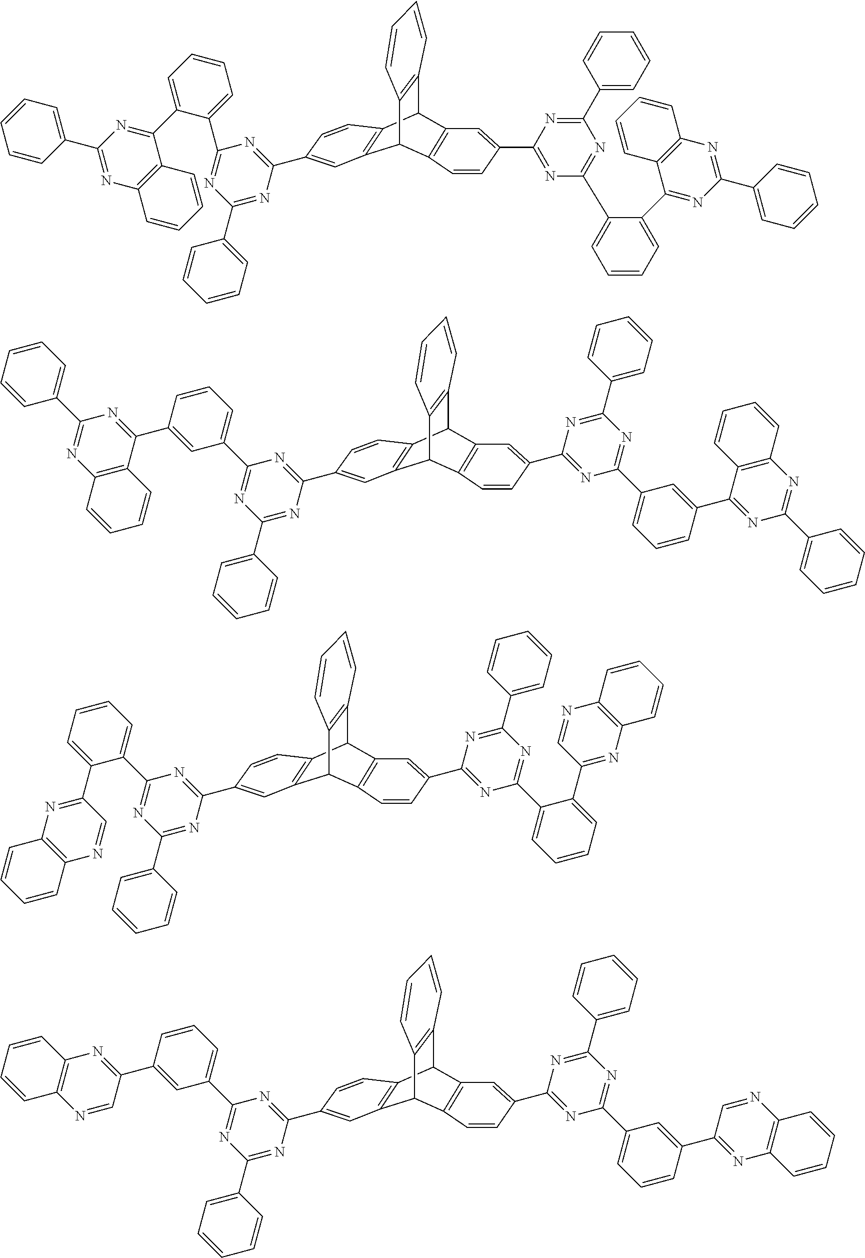

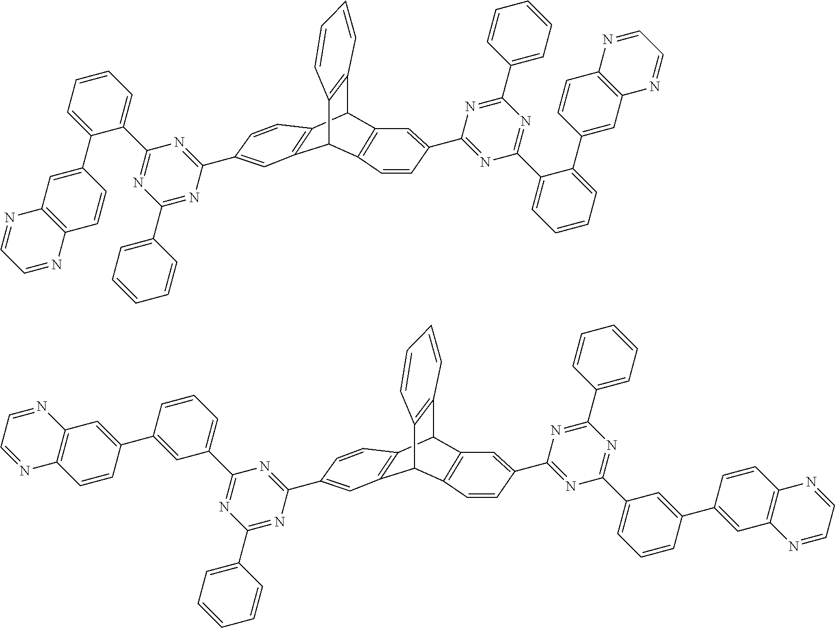 [SMILES: c1ccc(-c2nc(-c3ccc4c(c3)C3c5ccccc5C4c4cc(-c5nc(-c6ccccc6)nc(-c6ccccc6-c6ccc7nccnc7c6)n5)ccc43)nc(-c3ccccc3-c3ccc4nccnc4c3)n2)cc1 ; c1ccc(-c2nc(-c3cccc(-c4ccc5nccnc5c4)c3)nc(-c3ccc4c(c3)C3c5ccccc5C4c4cc(-c5nc(-c6ccccc6)nc(-c6cccc(-c7ccc8nccnc8c7)c6)n5)ccc43)n2)cc1]

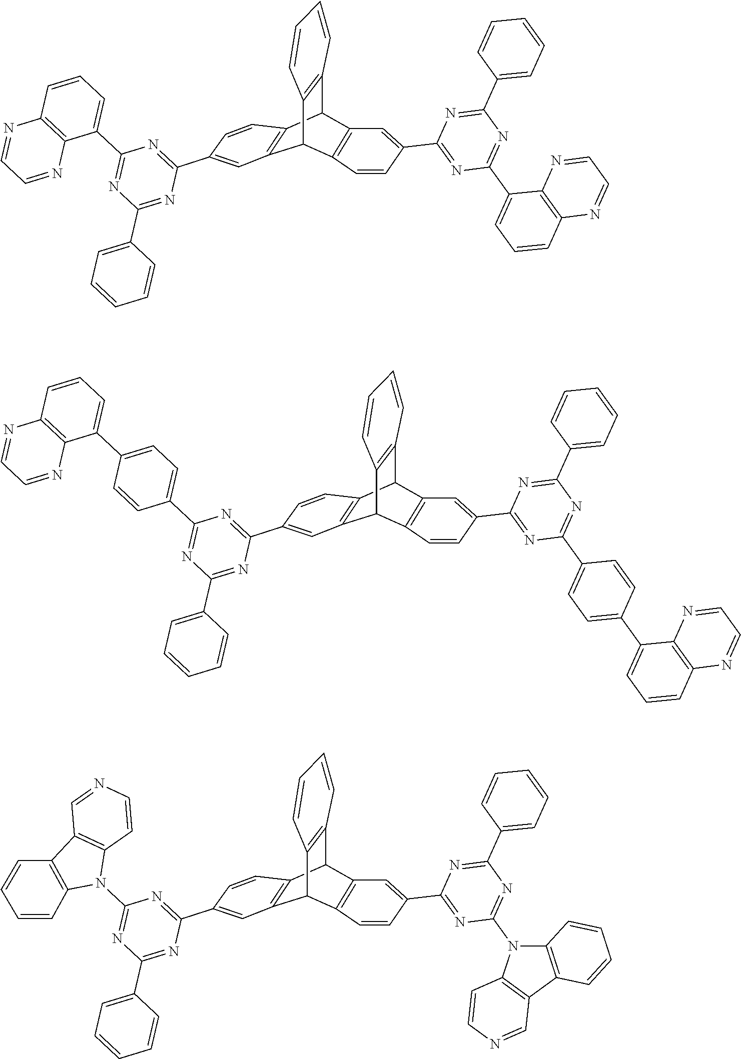 [SMILES: c1ccc(-c2nc(-c3ccc4c(c3)C3c5ccccc5C4c4cc(-c5nc(-c6ccccc6)nc(-c6cccc7nccnc67)n5)ccc43)nc(-c3cccc4nccnc34)n2)cc1 ; c1ccc(-c2nc(-c3ccc(-c4cccc5nccnc45)cc3)nc(-c3ccc4c(c3)C3c5ccccc5C4c4cc(-c5nc(-c6ccccc6)nc(-c6ccc(-c7cccc8nccnc78)cc6)n5)ccc43)n2)cc1 ; c1ccc(-c2nc(-c3ccc4c(c3)C3c5ccccc5C4c4cc(-c5nc(-c6ccccc6)nc(-n6c7ccccc7c7cnccc76)n5)ccc43)nc(-n3c4ccccc4c4cnccc43)n2)cc1]

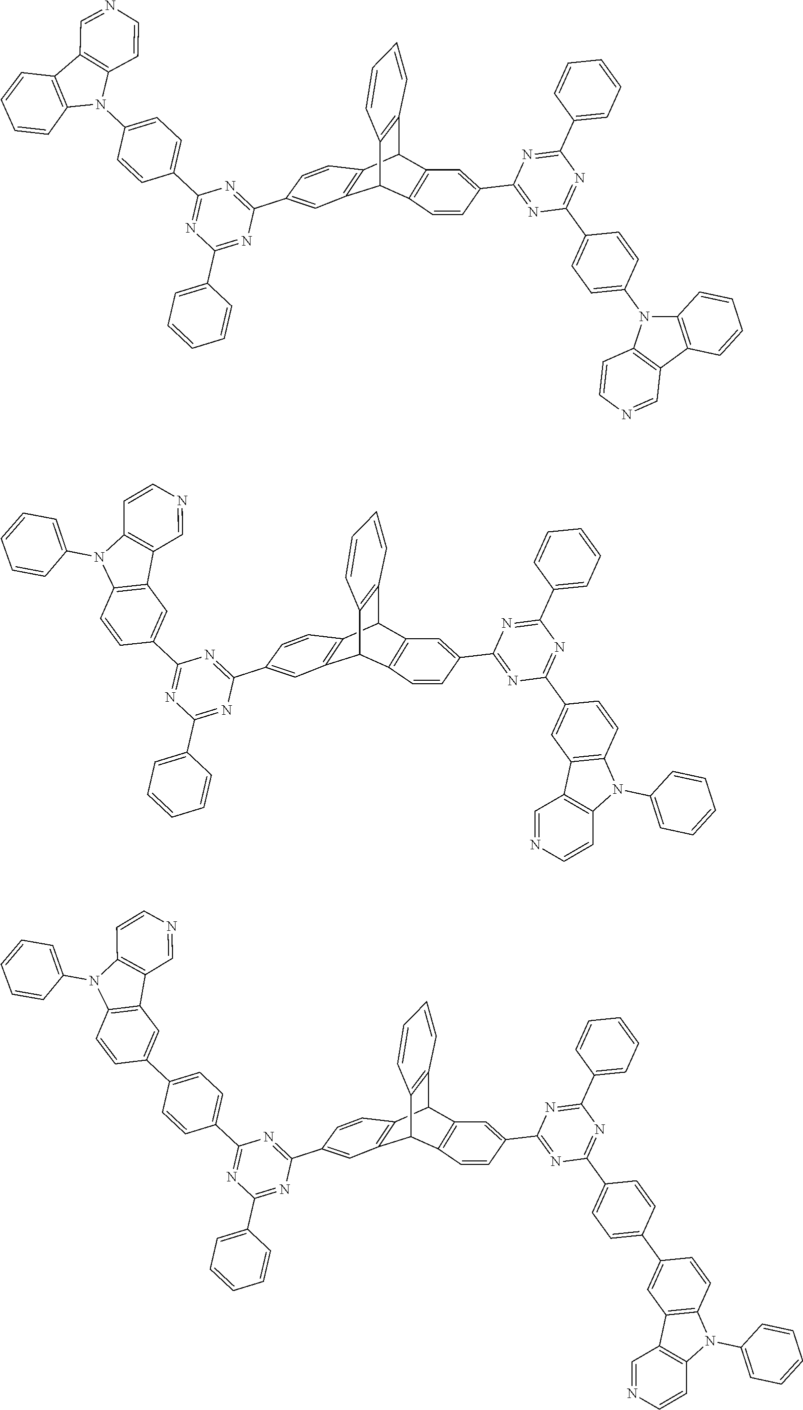 [SMILES: c1ccc(-c2nc(-c3ccc(-n4c5ccccc5c5cnccc54)cc3)nc(-c3ccc4c(c3)C3c5ccccc5C4c4cc(-c5nc(-c6ccccc6)nc(-c6ccc(-n7c8ccccc8c8cnccc87)cc6)n5)ccc43)n2)cc1 ; c1ccc(-c2nc(-c3ccc4c(c3)C3c5ccccc5C4c4cc(-c5nc(-c6ccccc6)nc(-c6ccc7c(c6)c6cnccc6n7-c6ccccc6)n5)ccc43)nc(-c3ccc4c(c3)c3cnccc3n4-c3ccccc3)n2)cc1 ; c1ccc(-c2nc(-c3ccc(-c4ccc5c(c4)c4cnccc4n5-c4ccccc4)cc3)nc(-c3ccc4c(c3)C3c5ccccc5C4c4cc(-c5nc(-c6ccccc6)nc(-c6ccc(-c7ccc8c(c7)c7cnccc7n8-c7ccccc7)cc6)n5)ccc43)n2)cc1]

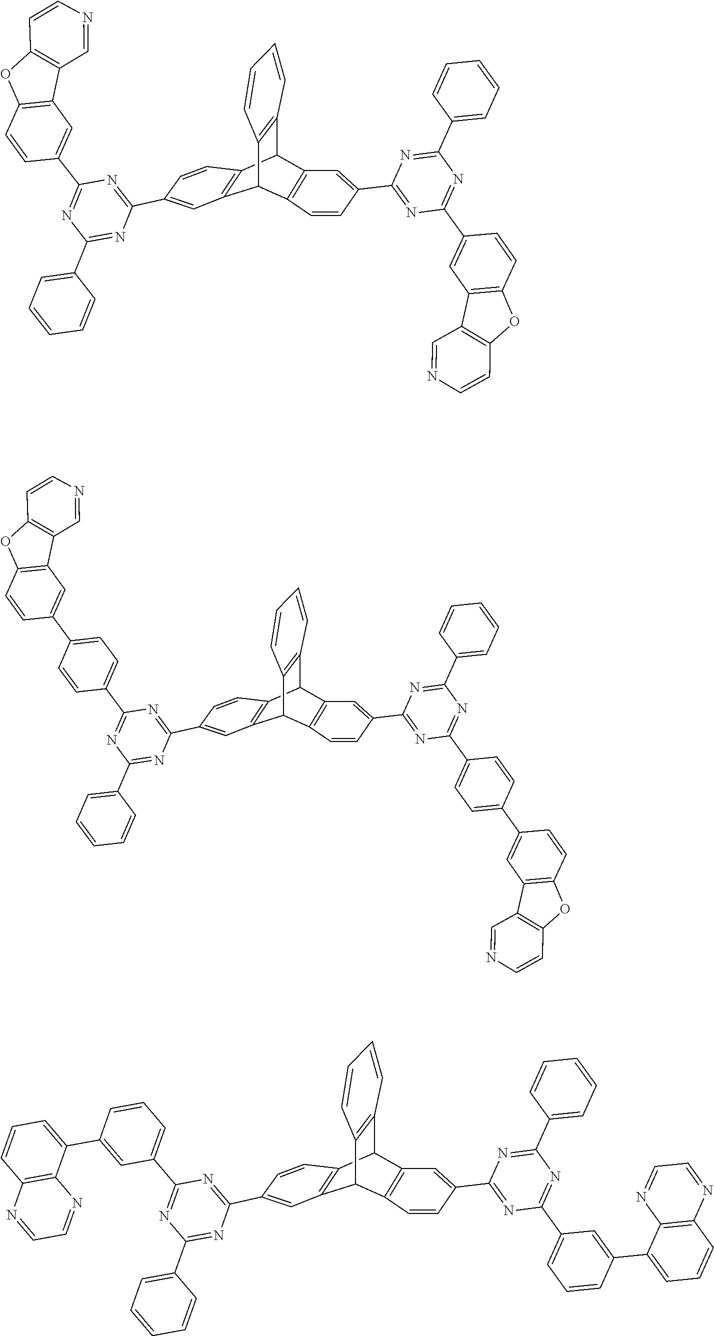 [SMILES: c1ccc(-c2nc(-c3ccc4c(c3)C3c5ccccc5C4c4cc(-c5nc(-c6ccccc6)nc(-c6ccc7oc8ccncc8c7c6)n5)ccc43)nc(-c3ccc4oc5ccncc5c4c3)n2)cc1 ; c1ccc(-c2nc(-c3ccc(-c4ccc5oc6ccncc6c5c4)cc3)nc(-c3ccc4c(c3)C3c5ccccc5C4c4cc(-c5nc(-c6ccccc6)nc(-c6ccc(-c7ccc8oc9ccncc9c8c7)cc6)n5)ccc43)n2)cc1 ; c1ccc(-c2nc(-c3cccc(-c4cccc5nccnc45)c3)nc(-c3ccc4c(c3)C3c5ccccc5C4c4cc(-c5nc(-c6ccccc6)nc(-c6cccc(-c7cccc8nccnc78)c6)n5)ccc43)n2)cc1]

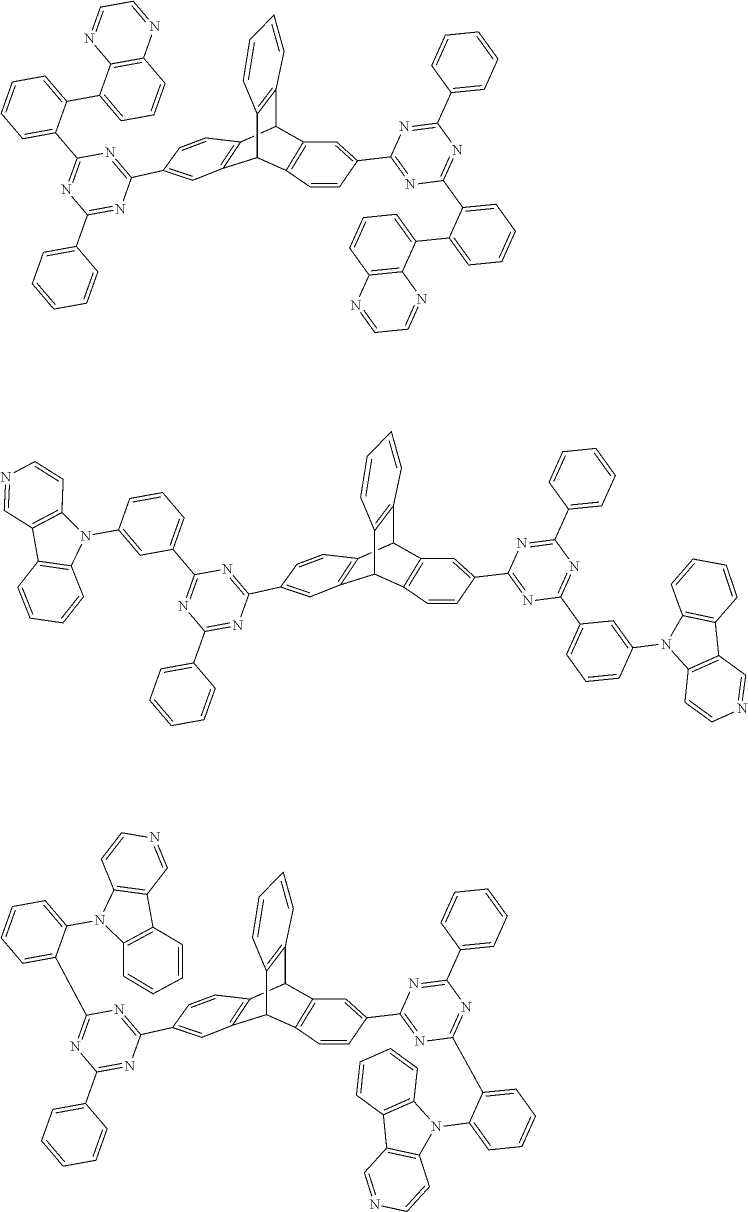 [SMILES: c1ccc(-c2nc(-c3ccc4c(c3)C3c5ccccc5C4c4cc(-c5nc(-c6ccccc6)nc(-c6ccccc6-c6cccc7nccnc67)n5)ccc43)nc(-c3ccccc3-c3cccc4nccnc34)n2)cc1 ; c1ccc(-c2nc(-c3cccc(-n4c5ccccc5c5cnccc54)c3)nc(-c3ccc4c(c3)C3c5ccccc5C4c4cc(-c5nc(-c6ccccc6)nc(-c6cccc(-n7c8ccccc8c8cnccc87)c6)n5)ccc43)n2)cc1 ; c1ccc(-c2nc(-c3ccc4c(c3)C3c5ccccc5C4c4cc(-c5nc(-c6ccccc6)nc(-c6ccccc6-n6c7ccccc7c7cnccc76)n5)ccc43)nc(-c3ccccc3-n3c4ccccc4c4cnccc43)n2)cc1]

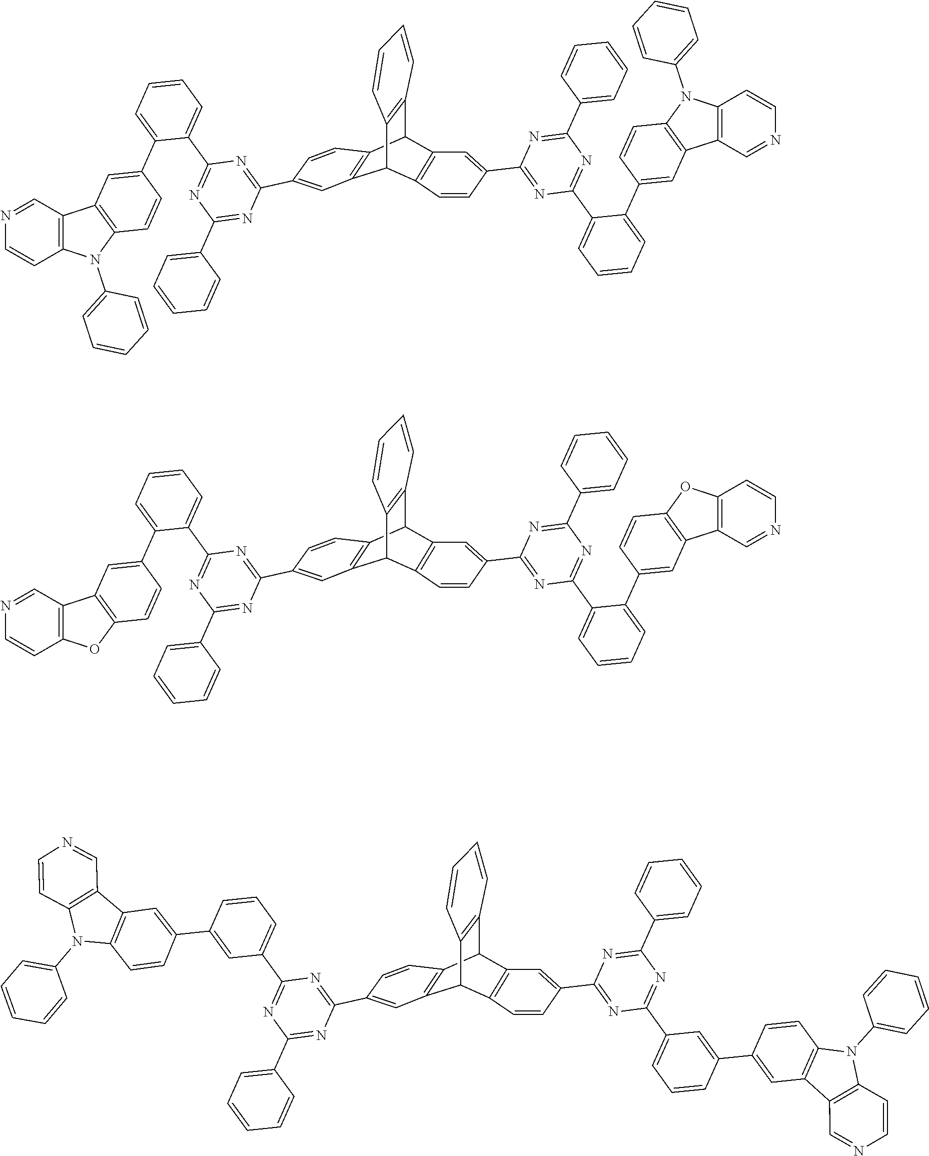 [SMILES: c1ccc(-c2nc(-c3ccc4c(c3)C3c5ccccc5C4c4cc(-c5nc(-c6ccccc6)nc(-c6ccccc6-c6ccc7c(c6)c6cnccc6n7-c6ccccc6)n5)ccc43)nc(-c3ccccc3-c3ccc4c(c3)c3cnccc3n4-c3ccccc3)n2)cc1 ; c1ccc(-c2nc(-c3ccc4c(c3)C3c5ccccc5C4c4cc(-c5nc(-c6ccccc6)nc(-c6ccccc6-c6ccc7oc8ccncc8c7c6)n5)ccc43)nc(-c3ccccc3-c3ccc4oc5ccncc5c4c3)n2)cc1 ; c1ccc(-c2nc(-c3cccc(-c4ccc5c(c4)c4cnccc4n5-c4ccccc4)c3)nc(-c3ccc4c(c3)C3c5ccccc5C4c4cc(-c5nc(-c6ccccc6)nc(-c6cccc(-c7ccc8c(c7)c7cnccc7n8-c7ccccc7)c6)n5)ccc43)n2)cc1]

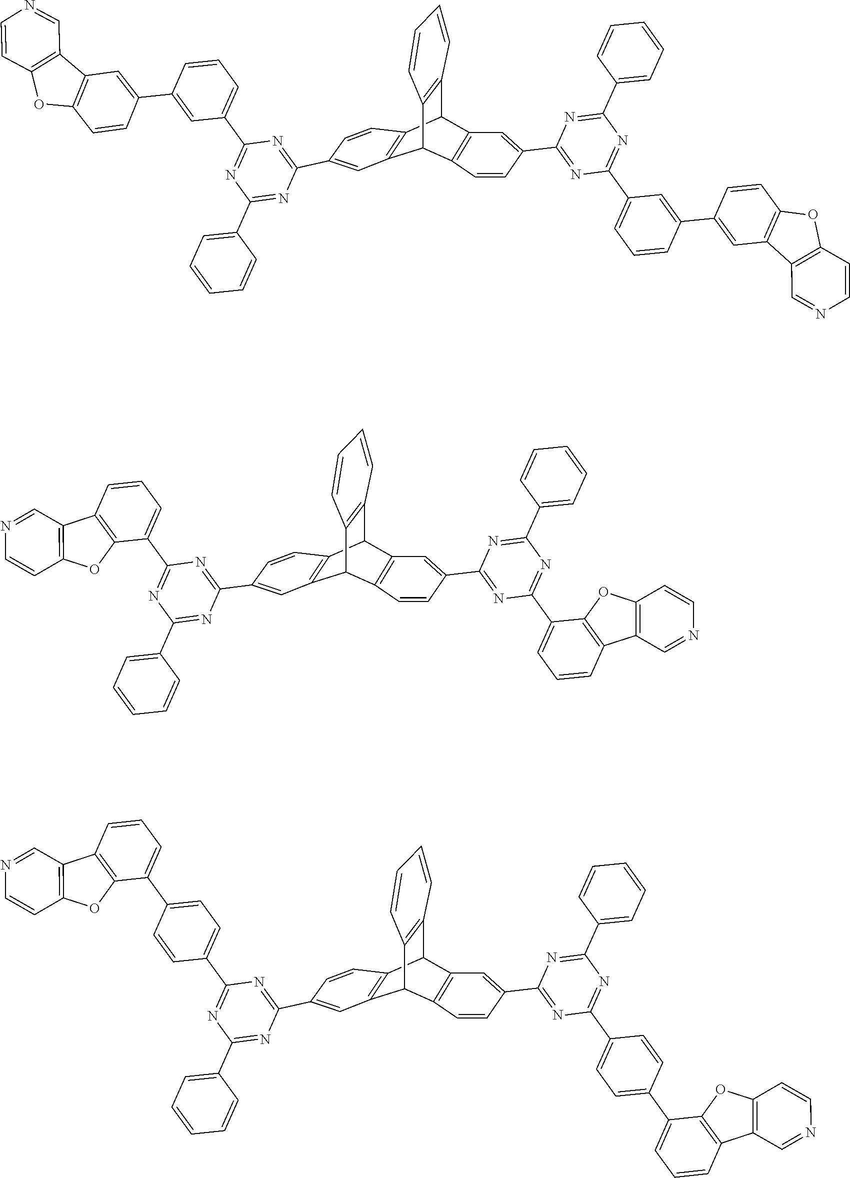 [SMILES: c1ccc(-c2nc(-c3cccc(-c4ccc5oc6ccncc6c5c4)c3)nc(-c3ccc4c(c3)C3c5ccccc5C4c4cc(-c5nc(-c6ccccc6)nc(-c6cccc(-c7ccc8oc9ccncc9c8c7)c6)n5)ccc43)n2)cc1 ; c1ccc(-c2nc(-c3ccc4c(c3)C3c5ccccc5C4c4cc(-c5nc(-c6ccccc6)nc(-c6cccc7c6oc6ccncc67)n5)ccc43)nc(-c3cccc4c3oc3ccncc34)n2)cc1 ; c1ccc(-c2nc(-c3ccc(-c4cccc5c4oc4ccncc45)cc3)nc(-c3ccc4c(c3)C3c5ccccc5C4c4cc(-c5nc(-c6ccccc6)nc(-c6ccc(-c7cccc8c7oc7ccncc78)cc6)n5)ccc43)n2)cc1]

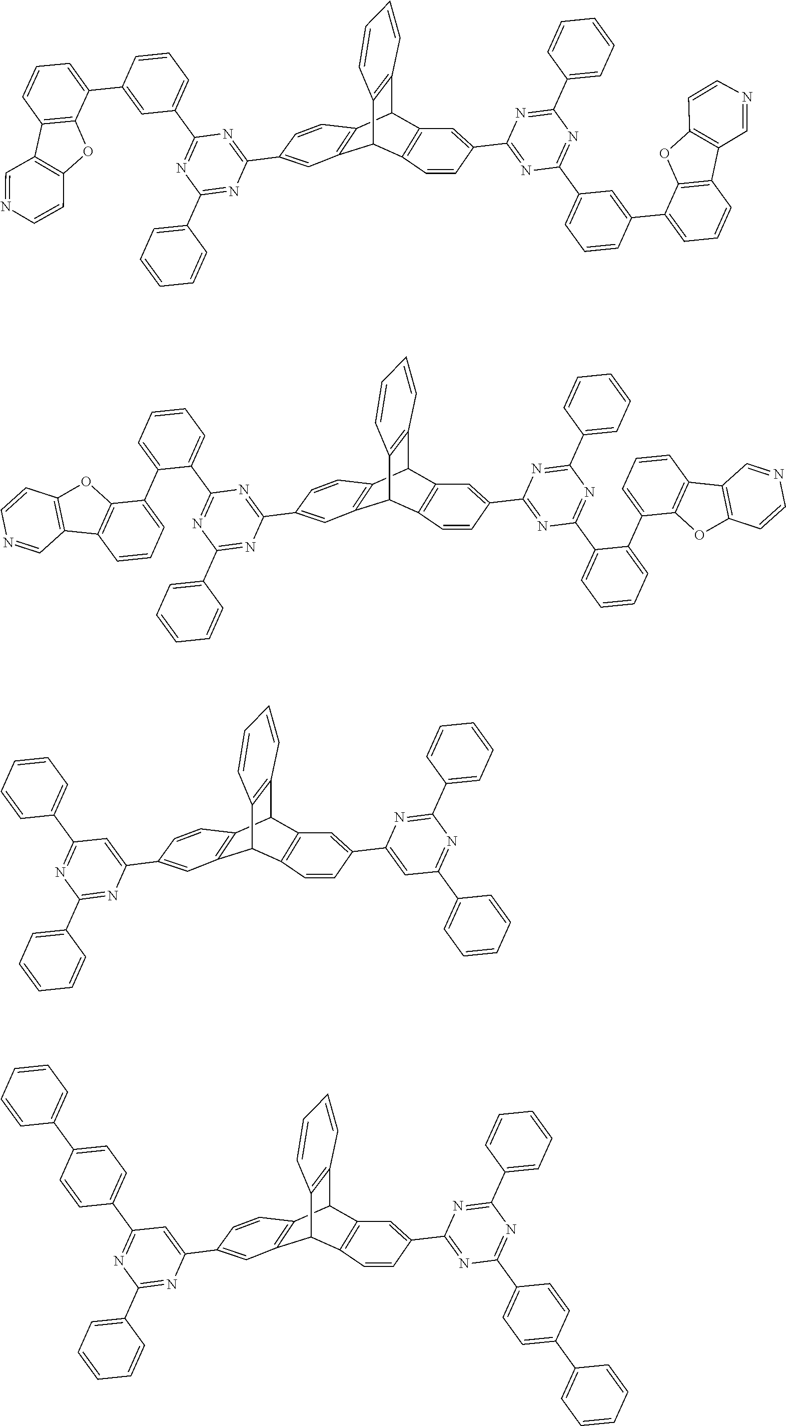

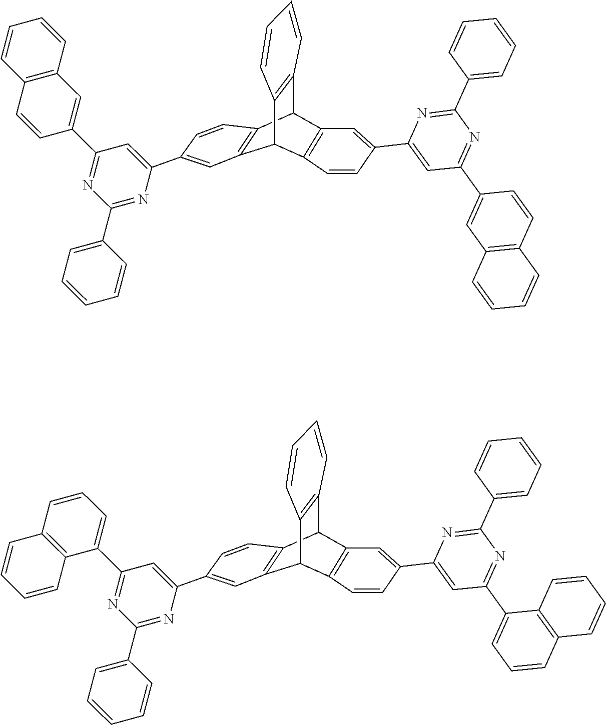 [SMILES: c1ccc(-c2nc(-c3ccc4c(c3)C3c5ccccc5C4c4cc(-c5cc(-c6ccc7ccccc7c6)nc(-c6ccccc6)n5)ccc43)cc(-c3ccc4ccccc4c3)n2)cc1 ; c1ccc(-c2nc(-c3ccc4c(c3)C3c5ccccc5C4c4cc(-c5cc(-c6cccc7ccccc67)nc(-c6ccccc6)n5)ccc43)cc(-c3cccc4ccccc34)n2)cc1]

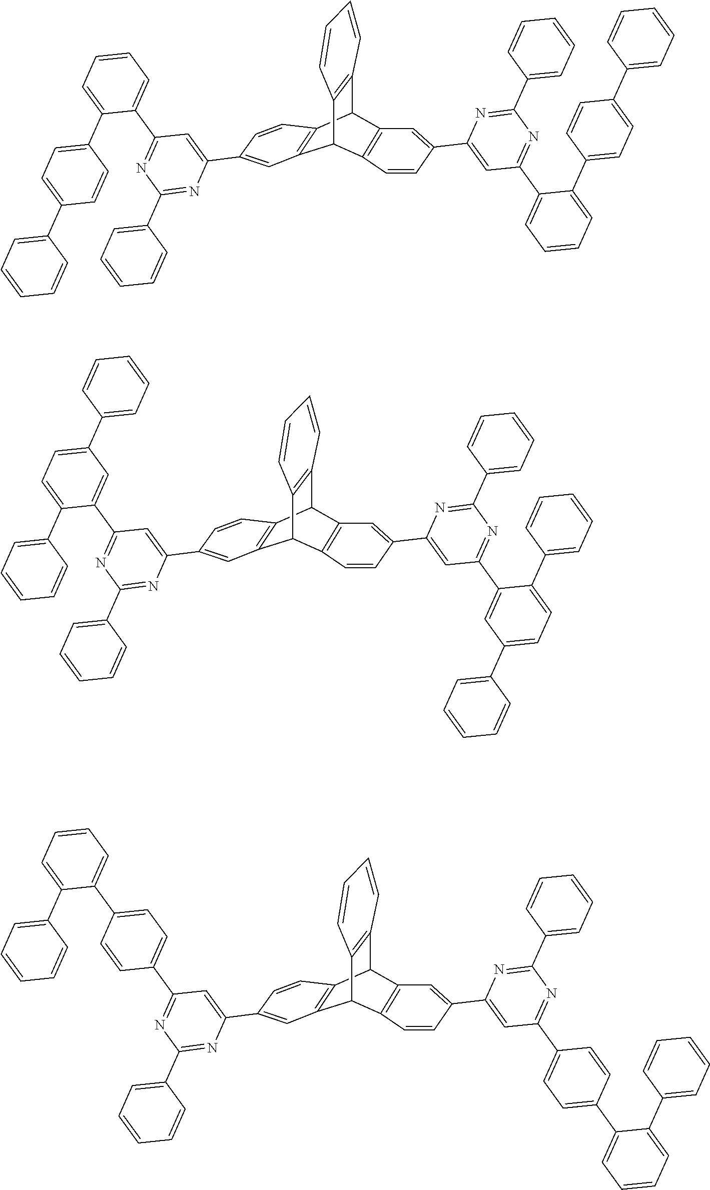 [SMILES: c1ccc(-c2ccc(-c3ccccc3-c3cc(-c4ccc5c(c4)C4c6ccccc6C5c5cc(-c6cc(-c7ccccc7-c7ccc(-c8ccccc8)cc7)nc(-c7ccccc7)n6)ccc54)nc(-c4ccccc4)n3)cc2)cc1 ; c1ccc(-c2ccc(-c3ccccc3)c(-c3cc(-c4ccc5c(c4)C4c6ccccc6C5c5cc(-c6cc(-c7cc(-c8ccccc8)ccc7-c7ccccc7)nc(-c7ccccc7)n6)ccc54)nc(-c4ccccc4)n3)c2)cc1 ; c1ccc(-c2nc(-c3ccc(-c4ccccc4-c4ccccc4)cc3)cc(-c3ccc4c(c3)C3c5ccccc5C4c4cc(-c5cc(-c6ccc(-c7ccccc7-c7ccccc7)cc6)nc(-c6ccccc6)n5)ccc43)n2)cc1]

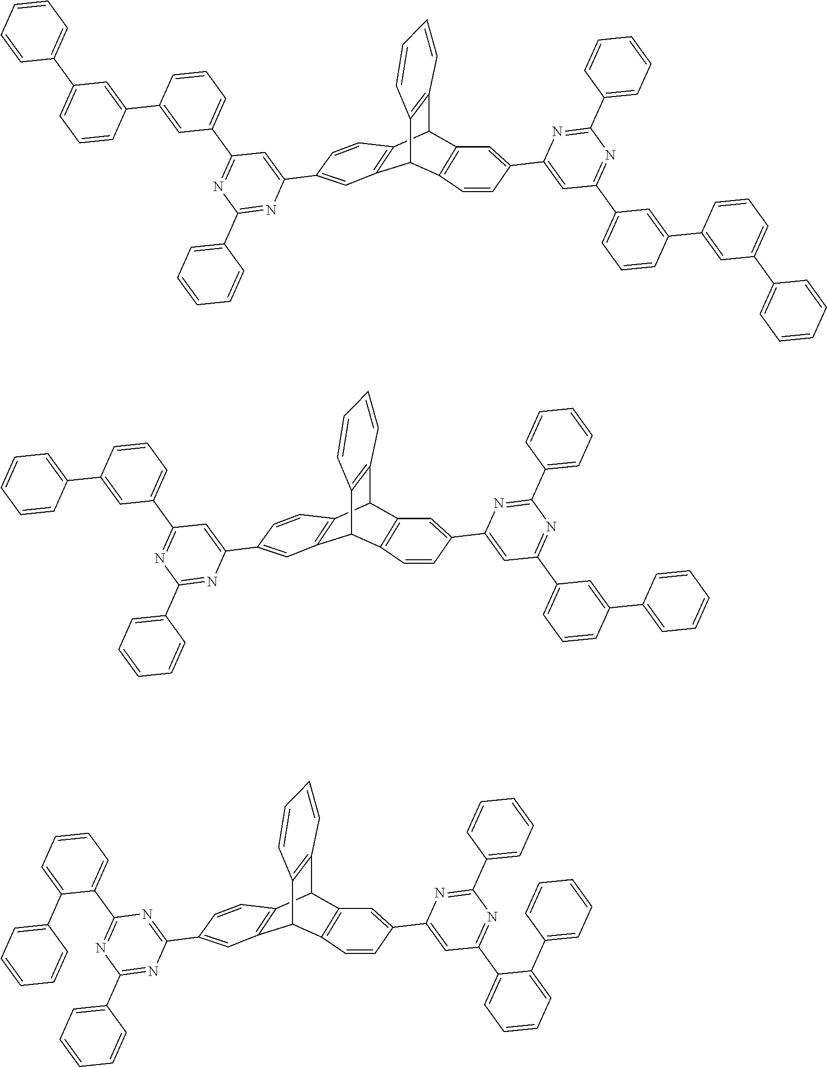 [SMILES: c1ccc(-c2cccc(-c3cccc(-c4cc(-c5ccc6c(c5)C5c7ccccc7C6c6cc(-c7cc(-c8cccc(-c9cccc(-c%10ccccc%10)c9)c8)nc(-c8ccccc8)n7)ccc65)nc(-c5ccccc5)n4)c3)c2)cc1 ; c1ccc(-c2cccc(-c3cc(-c4ccc5c(c4)C4c6ccccc6C5c5cc(-c6cc(-c7cccc(-c8ccccc8)c7)nc(-c7ccccc7)n6)ccc54)nc(-c4ccccc4)n3)c2)cc1 ; c1ccc(-c2nc(-c3ccc4c(c3)C3c5ccccc5C4c4cc(-c5nc(-c6ccccc6)nc(-c6ccccc6-c6ccccc6)n5)ccc43)cc(-c3ccccc3-c3ccccc3)n2)cc1]

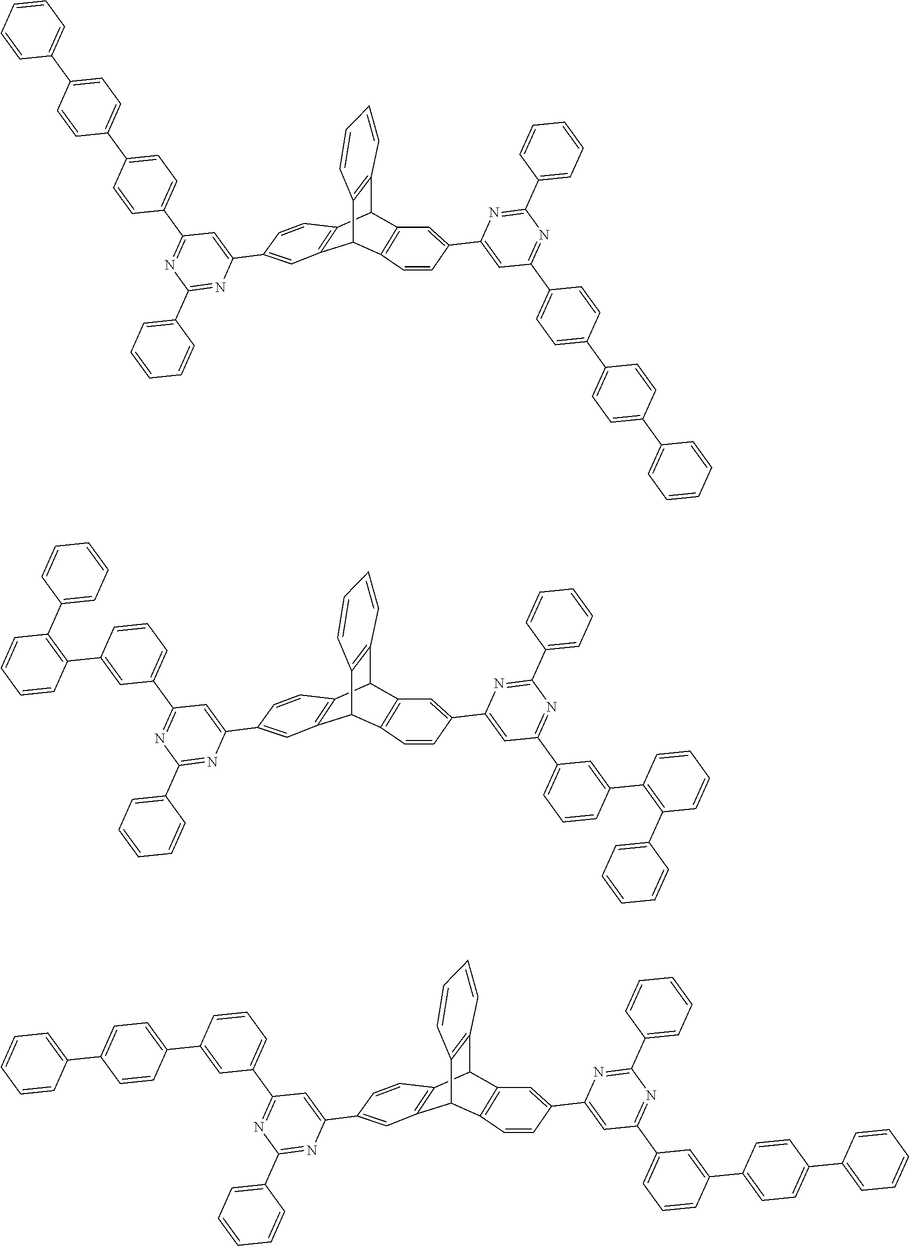 [SMILES: c1ccc(-c2ccc(-c3ccc(-c4cc(-c5ccc6c(c5)C5c7ccccc7C6c6cc(-c7cc(-c8ccc(-c9ccc(-c%10ccccc%10)cc9)cc8)nc(-c8ccccc8)n7)ccc65)nc(-c5ccccc5)n4)cc3)cc2)cc1 ; c1ccc(-c2nc(-c3cccc(-c4ccccc4-c4ccccc4)c3)cc(-c3ccc4c(c3)C3c5ccccc5C4c4cc(-c5cc(-c6cccc(-c7ccccc7-c7ccccc7)c6)nc(-c6ccccc6)n5)ccc43)n2)cc1 ; c1ccc(-c2ccc(-c3cccc(-c4cc(-c5ccc6c(c5)C5c7ccccc7C6c6cc(-c7cc(-c8cccc(-c9ccc(-c%10ccccc%10)cc9)c8)nc(-c8ccccc8)n7)ccc65)nc(-c5ccccc5)n4)c3)cc2)cc1]

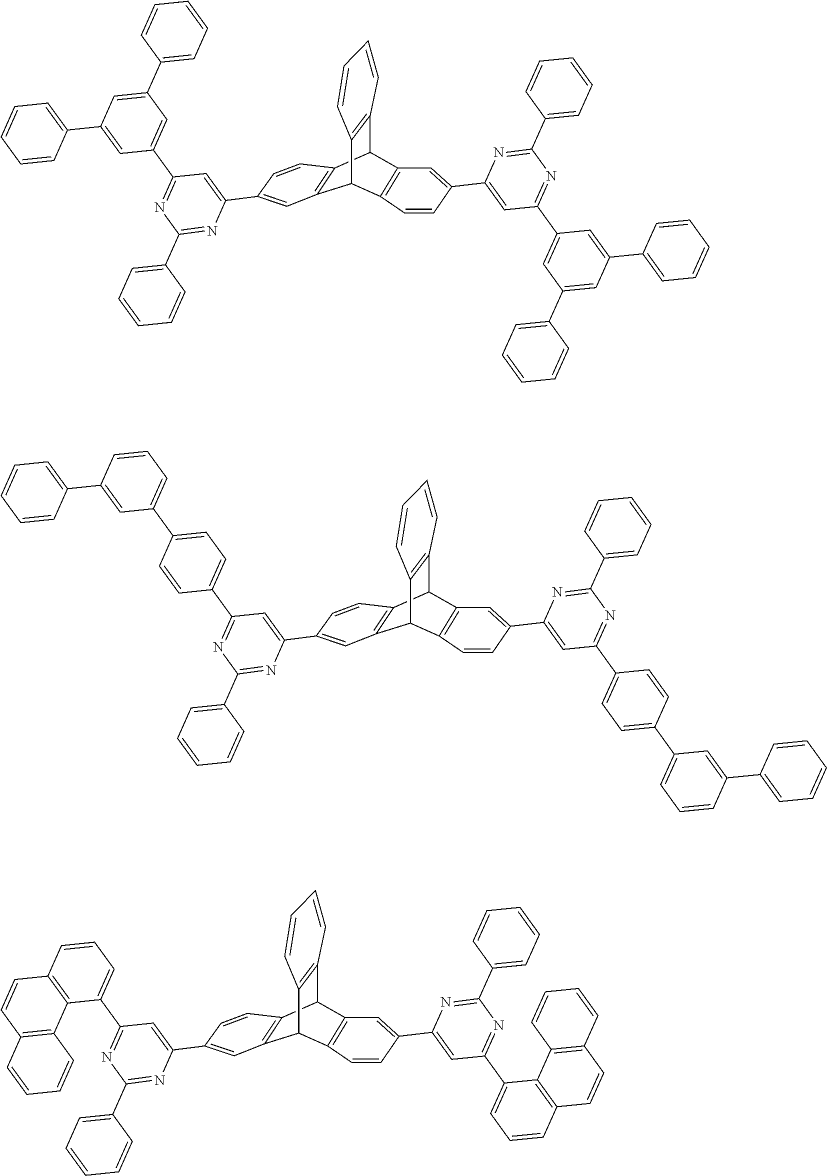 [SMILES: c1ccc(-c2cc(-c3ccccc3)cc(-c3cc(-c4ccc5c(c4)C4c6ccccc6C5c5cc(-c6cc(-c7cc(-c8ccccc8)cc(-c8ccccc8)c7)nc(-c7ccccc7)n6)ccc54)nc(-c4ccccc4)n3)c2)cc1 ; c1ccc(-c2cccc(-c3ccc(-c4cc(-c5ccc6c(c5)C5c7ccccc7C6c6cc(-c7cc(-c8ccc(-c9cccc(-c%10ccccc%10)c9)cc8)nc(-c8ccccc8)n7)ccc65)nc(-c5ccccc5)n4)cc3)c2)cc1 ; c1ccc(-c2nc(-c3ccc4c(c3)C3c5ccccc5C4c4cc(-c5cc(-c6cccc7ccc8ccccc8c67)nc(-c6ccccc6)n5)ccc43)cc(-c3cccc4ccc5ccccc5c34)n2)cc1]

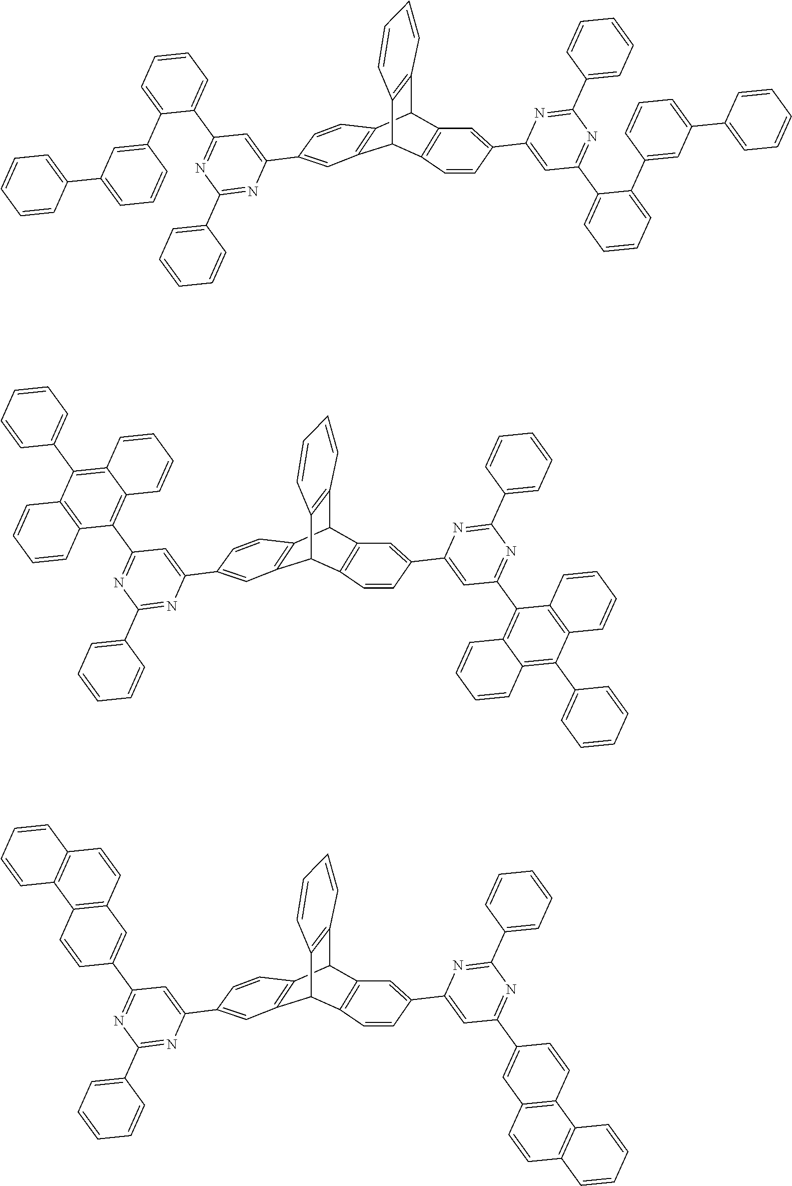 [SMILES: c1ccc(-c2cccc(-c3ccccc3-c3cc(-c4ccc5c(c4)C4c6ccccc6C5c5cc(-c6cc(-c7ccccc7-c7cccc(-c8ccccc8)c7)nc(-c7ccccc7)n6)ccc54)nc(-c4ccccc4)n3)c2)cc1 ; c1ccc(-c2nc(-c3ccc4c(c3)C3c5ccccc5C4c4cc(-c5cc(-c6c7ccccc7c(-c7ccccc7)c7ccccc67)nc(-c6ccccc6)n5)ccc43)cc(-c3c4ccccc4c(-c4ccccc4)c4ccccc34)n2)cc1 ; c1ccc(-c2nc(-c3ccc4c(c3)C3c5ccccc5C4c4cc(-c5cc(-c6ccc7c(ccc8ccccc87)c6)nc(-c6ccccc6)n5)ccc43)cc(-c3ccc4c(ccc5ccccc54)c3)n2)cc1]

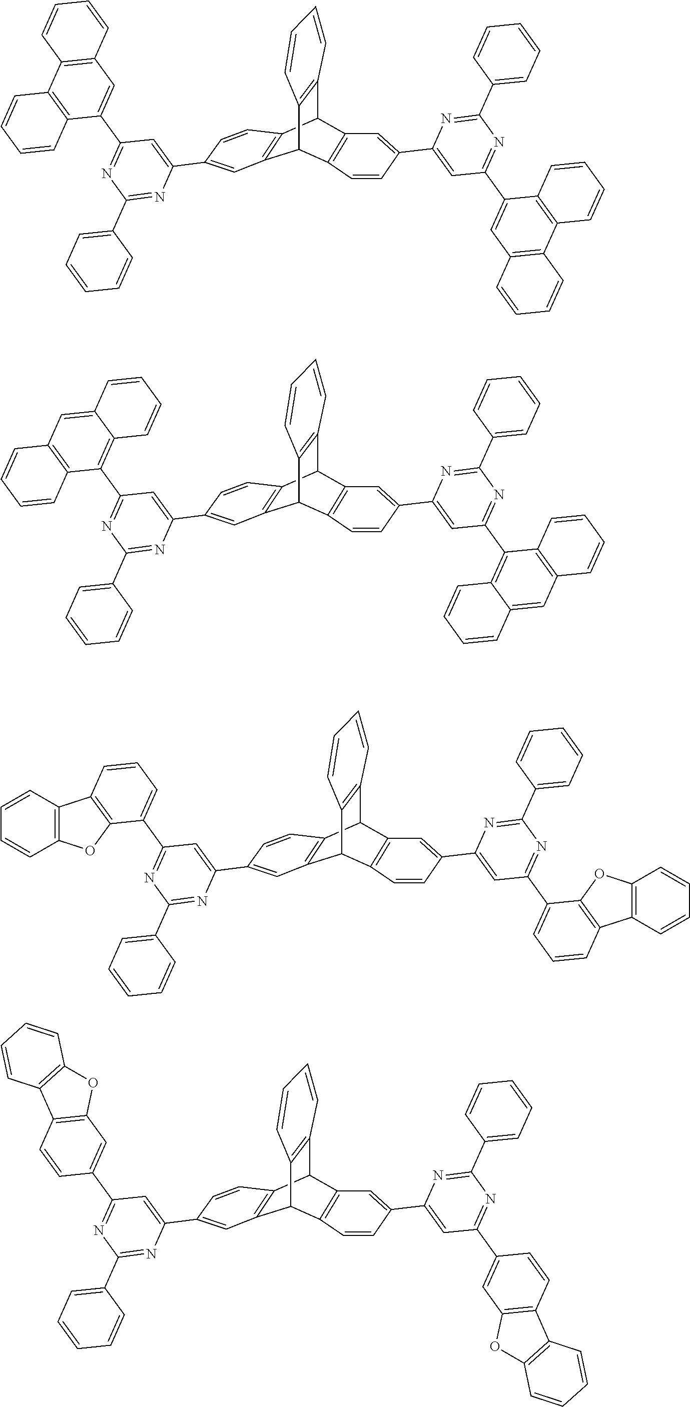 [SMILES: c1ccc(-c2nc(-c3ccc4c(c3)C3c5ccccc5C4c4cc(-c5cc(-c6cc7ccccc7c7ccccc67)nc(-c6ccccc6)n5)ccc43)cc(-c3cc4ccccc4c4ccccc34)n2)cc1 ; c1ccc(-c2nc(-c3ccc4c(c3)C3c5ccccc5C4c4cc(-c5cc(-c6c7ccccc7cc7ccccc67)nc(-c6ccccc6)n5)ccc43)cc(-c3c4ccccc4cc4ccccc34)n2)cc1 ; c1ccc(-c2nc(-c3ccc4c(c3)C3c5ccccc5C4c4cc(-c5cc(-c6cccc7c6oc6ccccc67)nc(-c6ccccc6)n5)ccc43)cc(-c3cccc4c3oc3ccccc34)n2)cc1 ; c1ccc(-c2nc(-c3ccc4c(c3)C3c5ccccc5C4c4cc(-c5cc(-c6ccc7c(c6)oc6ccccc67)nc(-c6ccccc6)n5)ccc43)cc(-c3ccc4c(c3)oc3ccccc34)n2)cc1]

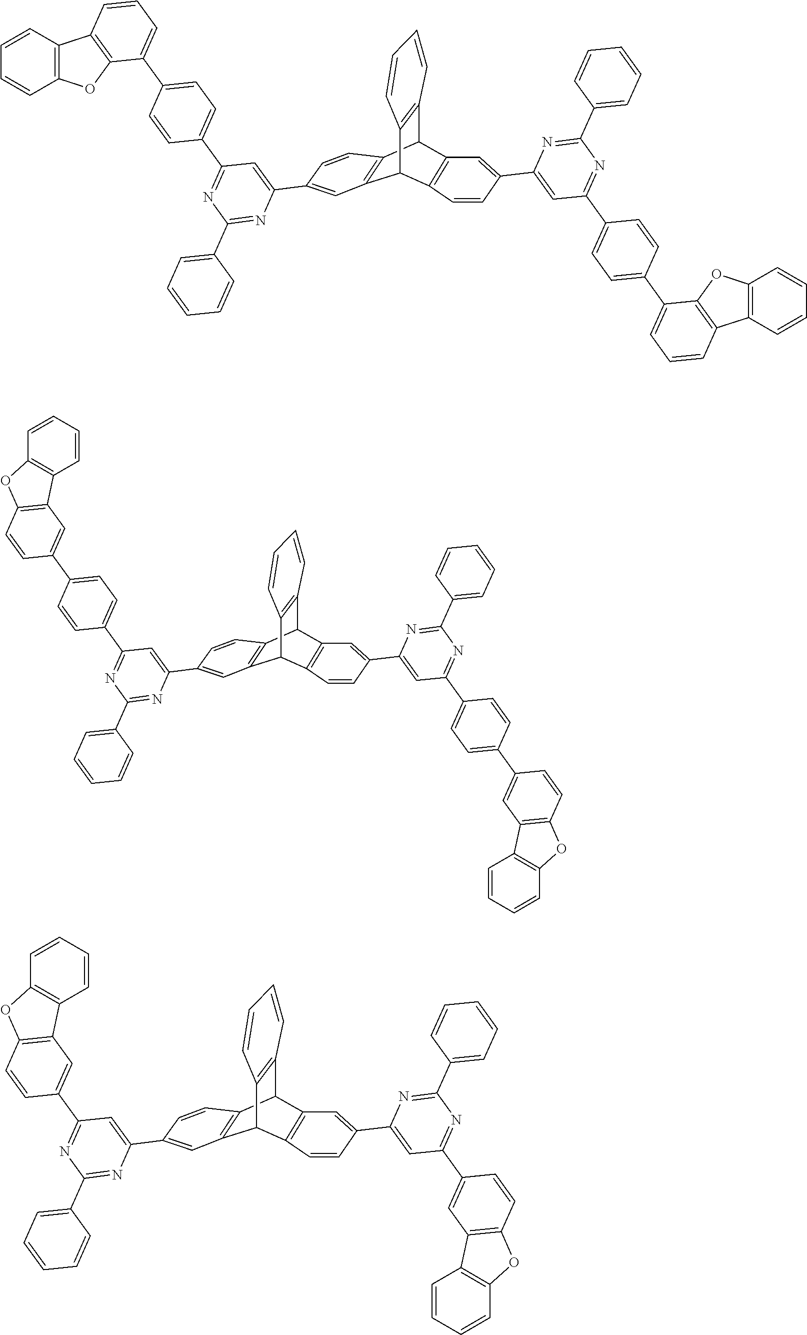 [SMILES: c1ccc(-c2nc(-c3ccc(-c4cccc5c4oc4ccccc45)cc3)cc(-c3ccc4c(c3)C3c5ccccc5C4c4cc(-c5cc(-c6ccc(-c7cccc8c7oc7ccccc78)cc6)nc(-c6ccccc6)n5)ccc43)n2)cc1 ; c1ccc(-c2nc(-c3ccc(-c4ccc5oc6ccccc6c5c4)cc3)cc(-c3ccc4c(c3)C3c5ccccc5C4c4cc(-c5cc(-c6ccc(-c7ccc8oc9ccccc9c8c7)cc6)nc(-c6ccccc6)n5)ccc43)n2)cc1 ; c1ccc(-c2nc(-c3ccc4c(c3)C3c5ccccc5C4c4cc(-c5cc(-c6ccc7oc8ccccc8c7c6)nc(-c6ccccc6)n5)ccc43)cc(-c3ccc4oc5ccccc5c4c3)n2)cc1]

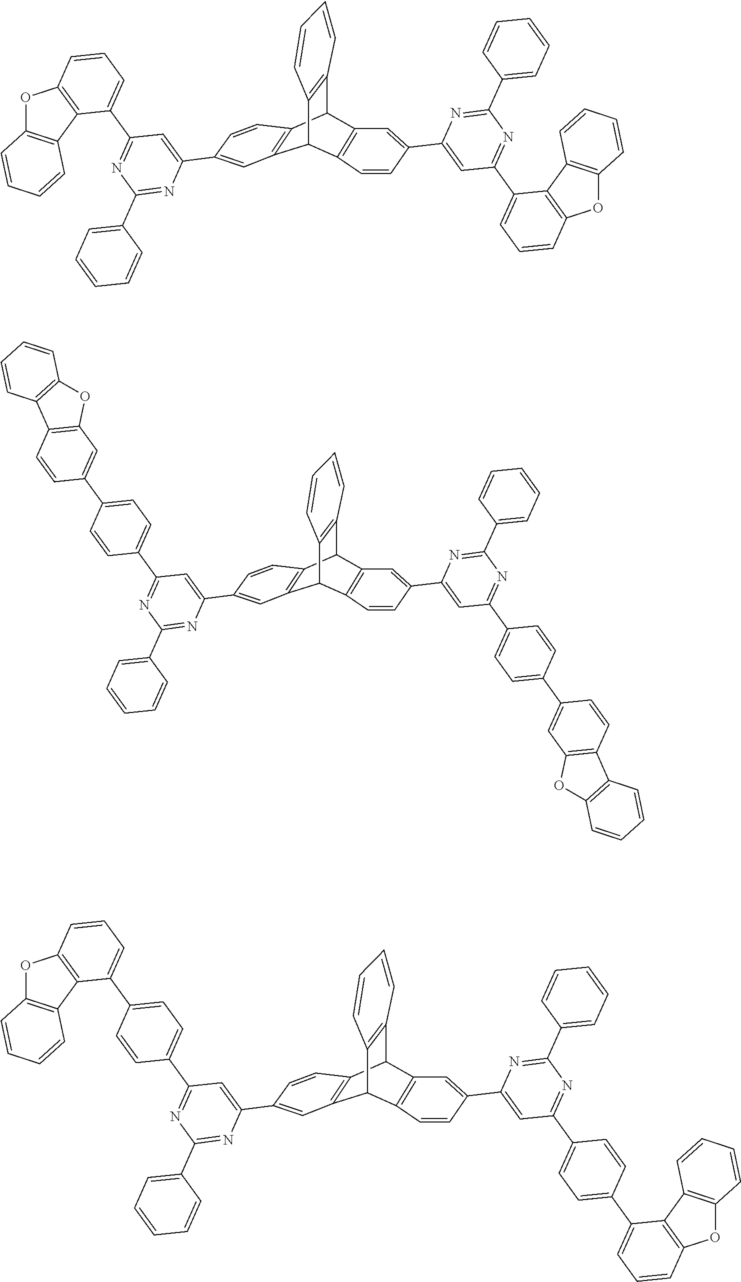 [SMILES: c1ccc(-c2nc(-c3ccc4c(c3)C3c5ccccc5C4c4cc(-c5cc(-c6cccc7oc8ccccc8c67)nc(-c6ccccc6)n5)ccc43)cc(-c3cccc4oc5ccccc5c34)n2)cc1 ; c1ccc(-c2nc(-c3ccc(-c4ccc5c(c4)oc4ccccc45)cc3)cc(-c3ccc4c(c3)C3c5ccccc5C4c4cc(-c5cc(-c6ccc(-c7ccc8c(c7)oc7ccccc78)cc6)nc(-c6ccccc6)n5)ccc43)n2)cc1 ; c1ccc(-c2nc(-c3ccc(-c4cccc5oc6ccccc6c45)cc3)cc(-c3ccc4c(c3)C3c5ccccc5C4c4cc(-c5cc(-c6ccc(-c7cccc8oc9ccccc9c78)cc6)nc(-c6ccccc6)n5)ccc43)n2)cc1]

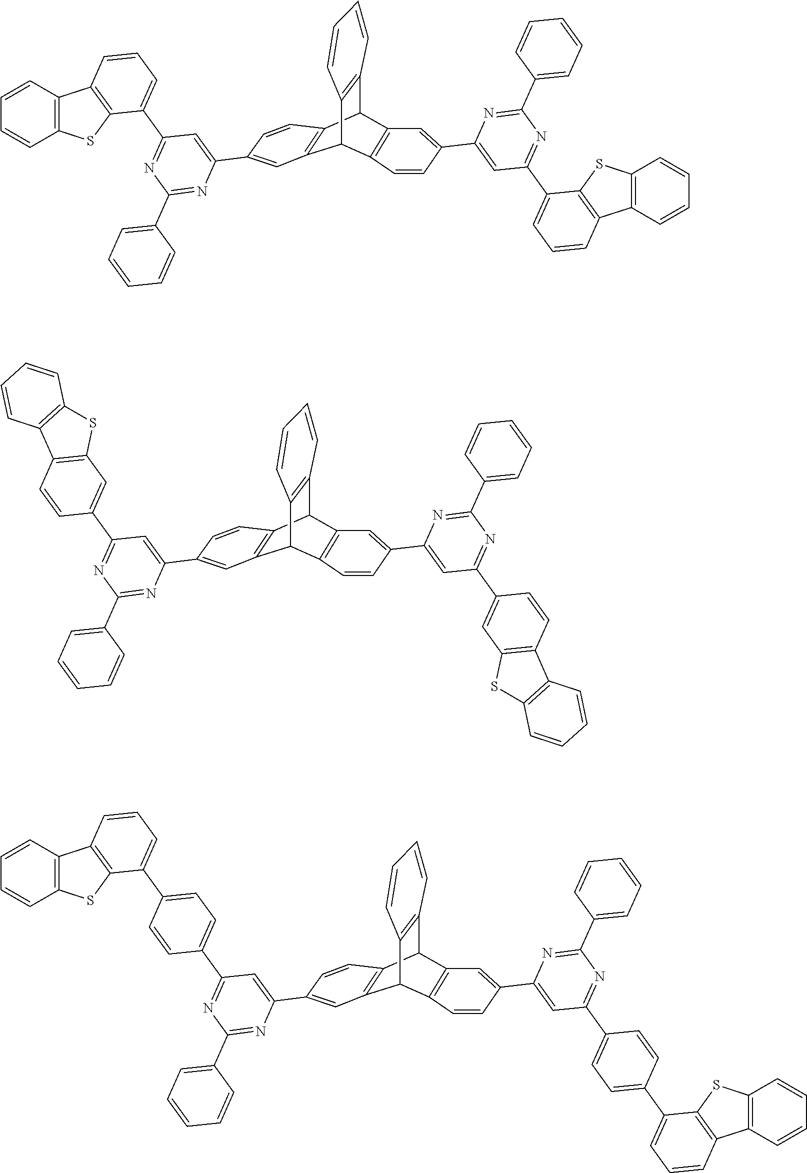 [SMILES: c1ccc(-c2nc(-c3ccc4c(c3)C3c5ccccc5C4c4cc(-c5cc(-c6cccc7c6sc6ccccc67)nc(-c6ccccc6)n5)ccc43)cc(-c3cccc4c3sc3ccccc34)n2)cc1 ; c1ccc(-c2nc(-c3ccc4c(c3)C3c5ccccc5C4c4cc(-c5cc(-c6ccc7c(c6)sc6ccccc67)nc(-c6ccccc6)n5)ccc43)cc(-c3ccc4c(c3)sc3ccccc34)n2)cc1 ; c1ccc(-c2nc(-c3ccc(-c4cccc5c4sc4ccccc45)cc3)cc(-c3ccc4c(c3)C3c5ccccc5C4c4cc(-c5cc(-c6ccc(-c7cccc8c7sc7ccccc78)cc6)nc(-c6ccccc6)n5)ccc43)n2)cc1]

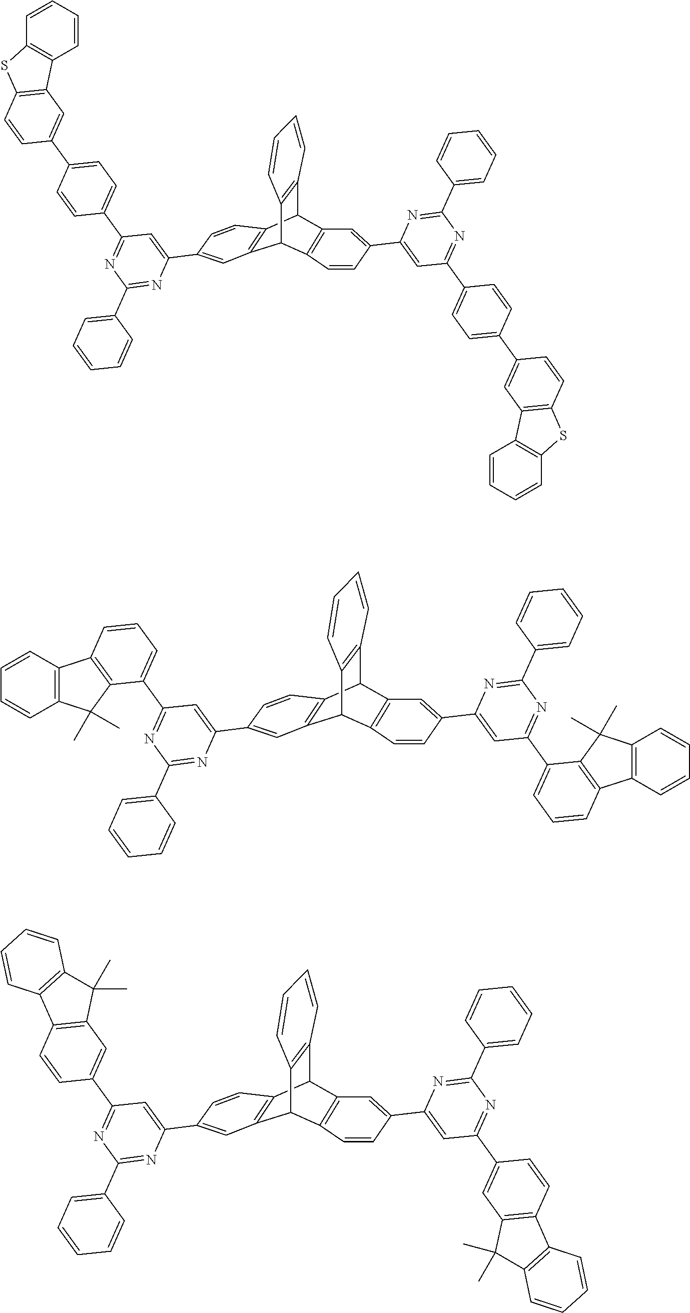 [SMILES: c1ccc(-c2nc(-c3ccc(-c4ccc5sc6ccccc6c5c4)cc3)cc(-c3ccc4c(c3)C3c5ccccc5C4c4cc(-c5cc(-c6ccc(-c7ccc8sc9ccccc9c8c7)cc6)nc(-c6ccccc6)n5)ccc43)n2)cc1 ; CC1(C)c2ccccc2-c2cccc(-c3cc(-c4ccc5c(c4)C4c6ccccc6C5c5cc(-c6cc(-c7cccc8c7C(C)(C)c7ccccc7-8)nc(-c7ccccc7)n6)ccc54)nc(-c4ccccc4)n3)c21 ; CC1(C)c2ccccc2-c2ccc(-c3cc(-c4ccc5c(c4)C4c6ccccc6C5c5cc(-c6cc(-c7ccc8c(c7)C(C)(C)c7ccccc7-8)nc(-c7ccccc7)n6)ccc54)nc(-c4ccccc4)n3)cc21]

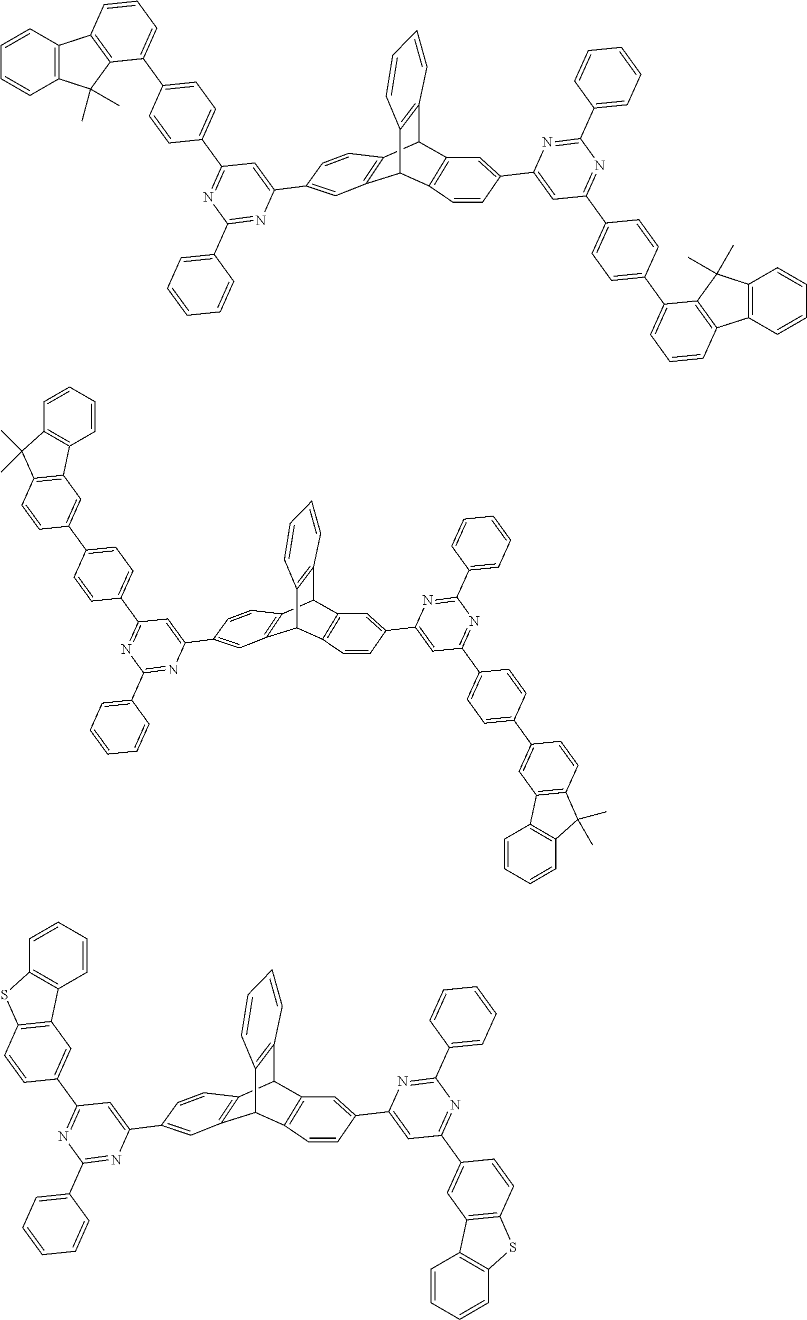

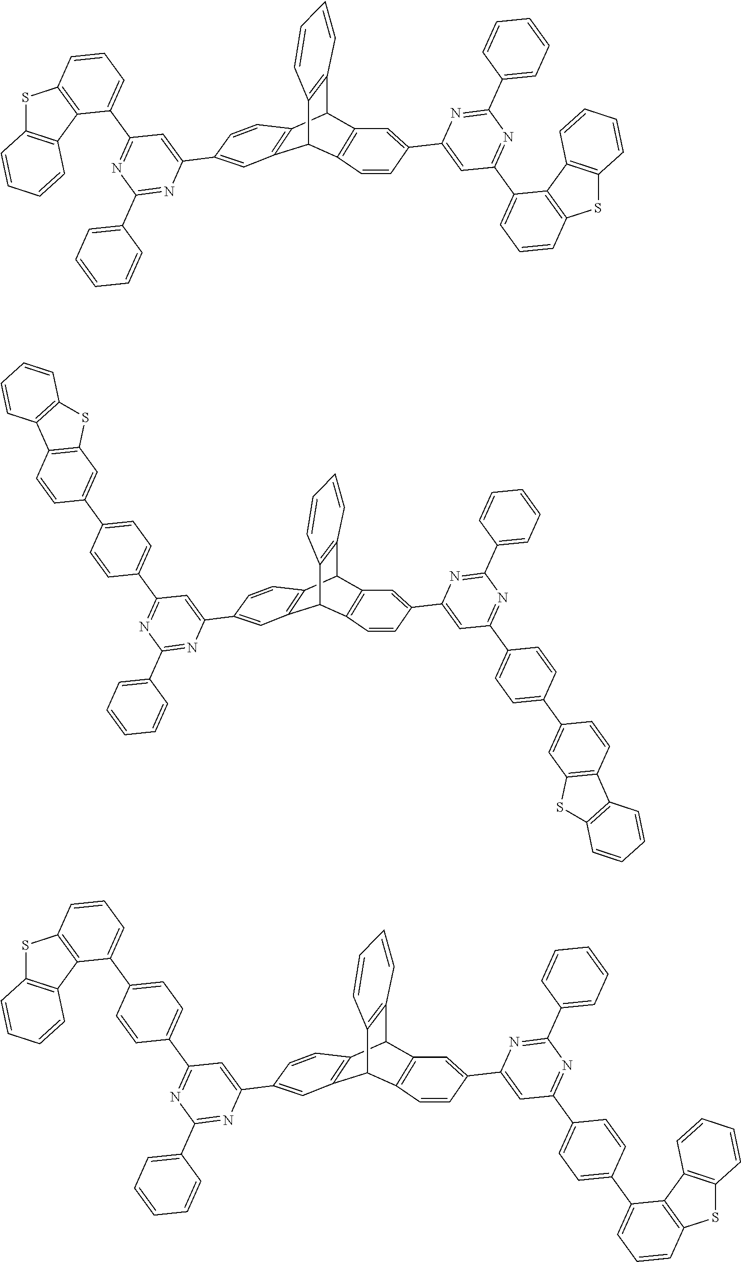 [SMILES: c1ccc(-c2nc(-c3ccc4c(c3)C3c5ccccc5C4c4cc(-c5cc(-c6cccc7sc8ccccc8c67)nc(-c6ccccc6)n5)ccc43)cc(-c3cccc4sc5ccccc5c34)n2)cc1 ; c1ccc(-c2nc(-c3ccc(-c4ccc5c(c4)sc4ccccc45)cc3)cc(-c3ccc4c(c3)C3c5ccccc5C4c4cc(-c5cc(-c6ccc(-c7ccc8c(c7)sc7ccccc78)cc6)nc(-c6ccccc6)n5)ccc43)n2)cc1 ; c1ccc(-c2nc(-c3ccc(-c4cccc5sc6ccccc6c45)cc3)cc(-c3ccc4c(c3)C3c5ccccc5C4c4cc(-c5cc(-c6ccc(-c7cccc8sc9ccccc9c78)cc6)nc(-c6ccccc6)n5)ccc43)n2)cc1]

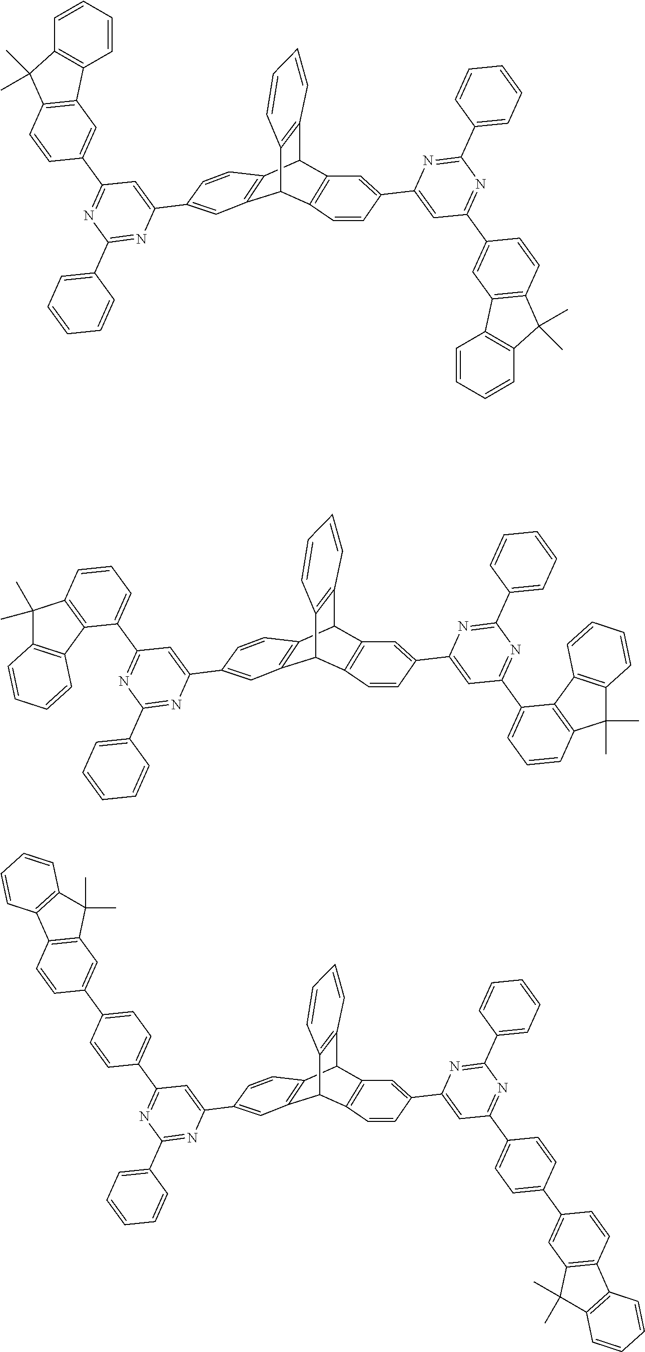

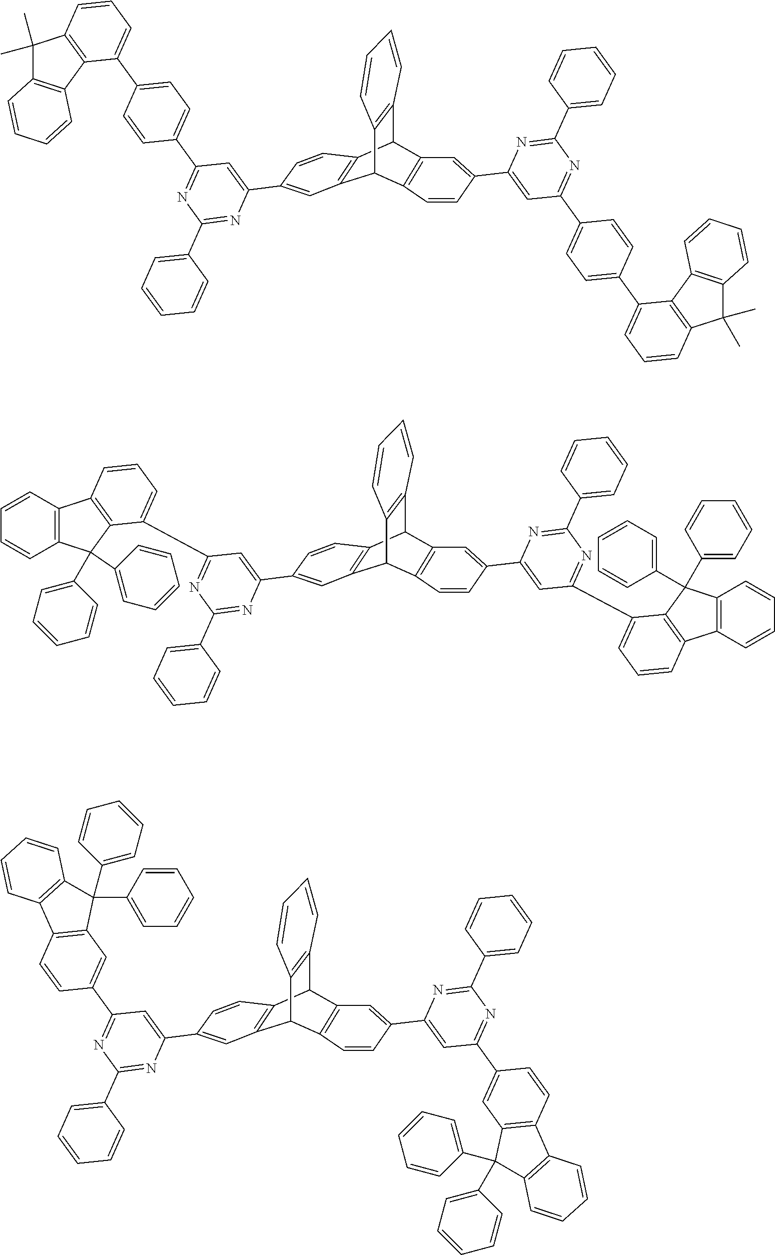

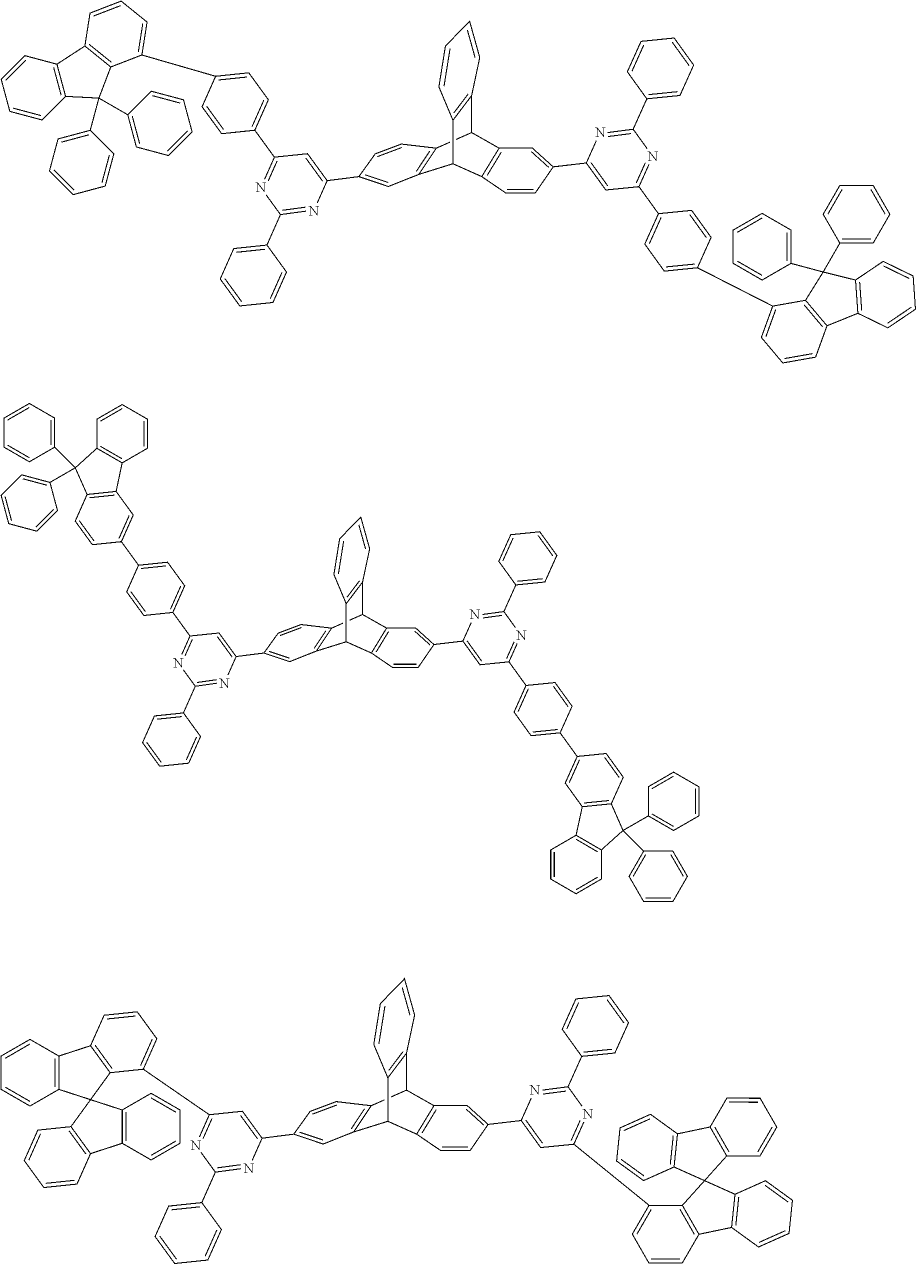

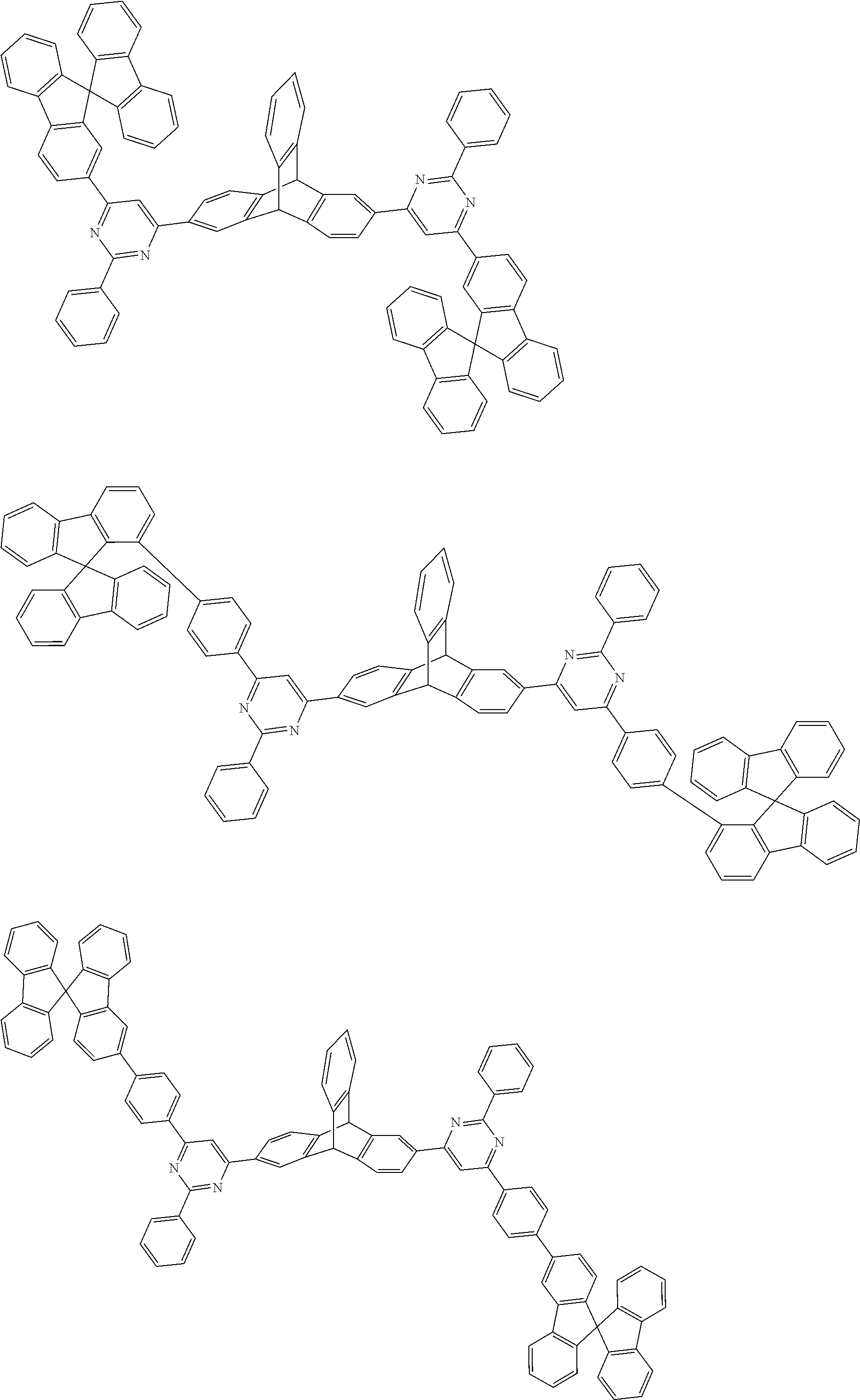

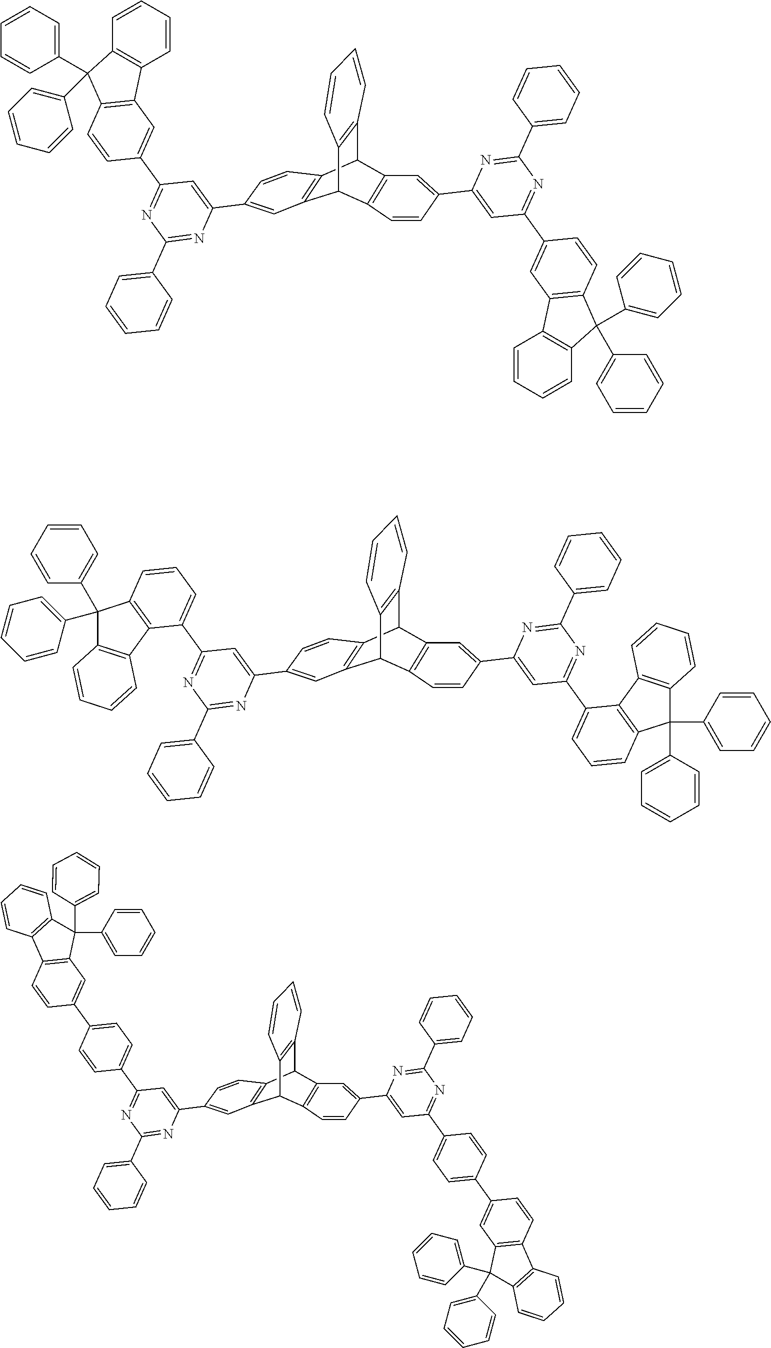

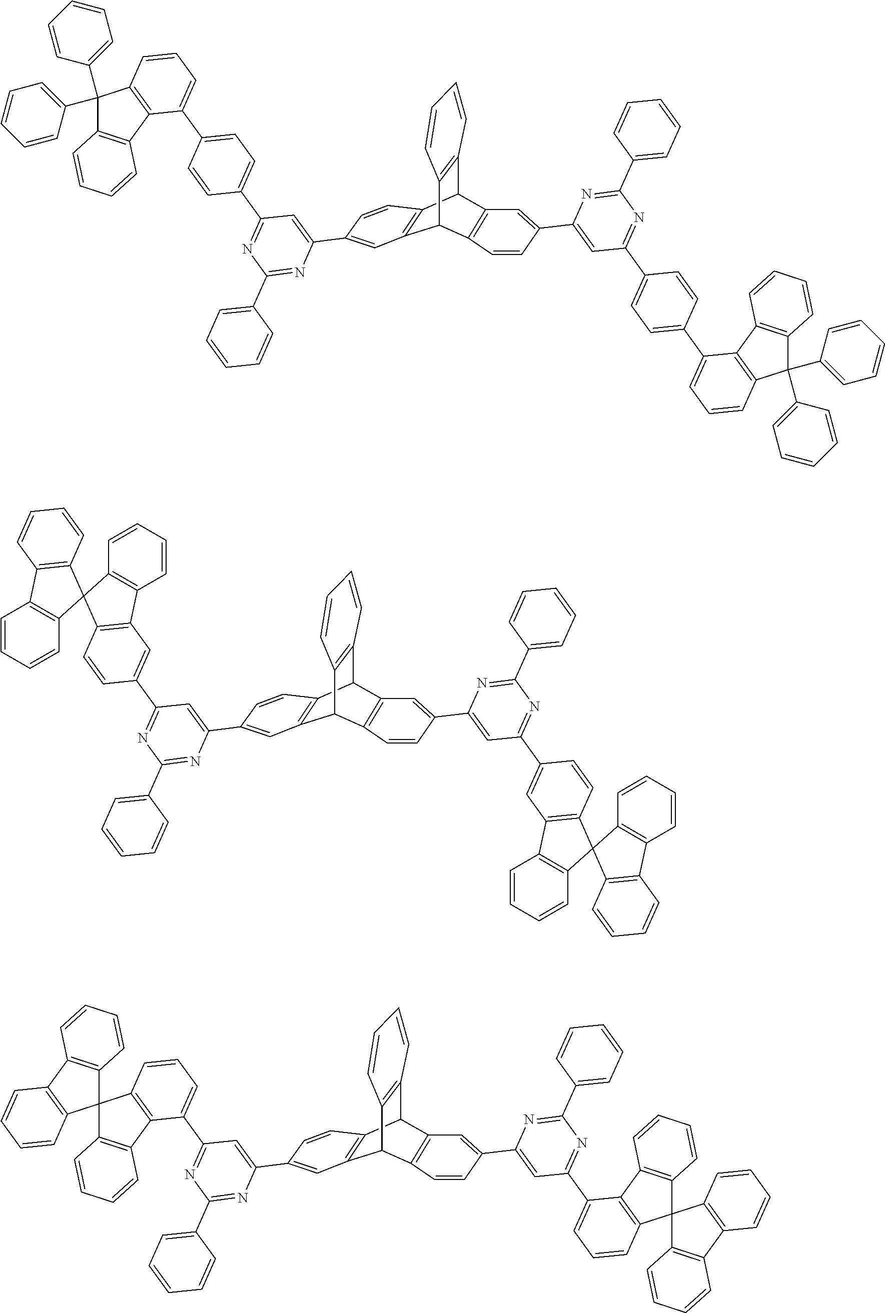

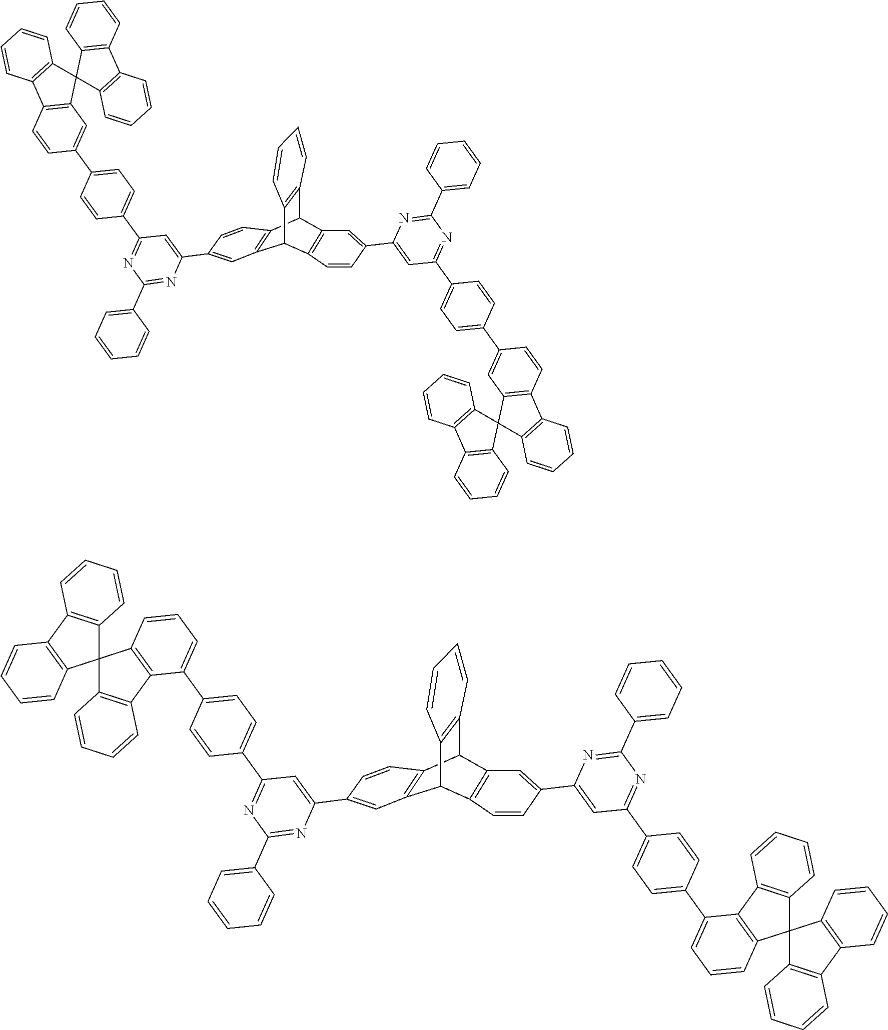 [SMILES: c1ccc(-c2nc(-c3ccc(-c4ccc5c(c4)C4(c6ccccc6-c6ccccc64)c4ccccc4-5)cc3)cc(-c3ccc4c(c3)C3c5ccccc5C4c4cc(-c5cc(-c6ccc(-c7ccc8c(c7)C7(c9ccccc9-c9ccccc97)c7ccccc7-8)cc6)nc(-c6ccccc6)n5)ccc43)n2)cc1 ; c1ccc(-c2nc(-c3ccc(-c4cccc5c4-c4ccccc4C54c5ccccc5-c5ccccc54)cc3)cc(-c3ccc4c(c3)C3c5ccccc5C4c4cc(-c5cc(-c6ccc(-c7cccc8c7-c7ccccc7C87c8ccccc8-c8ccccc87)cc6)nc(-c6ccccc6)n5)ccc43)n2)cc1]

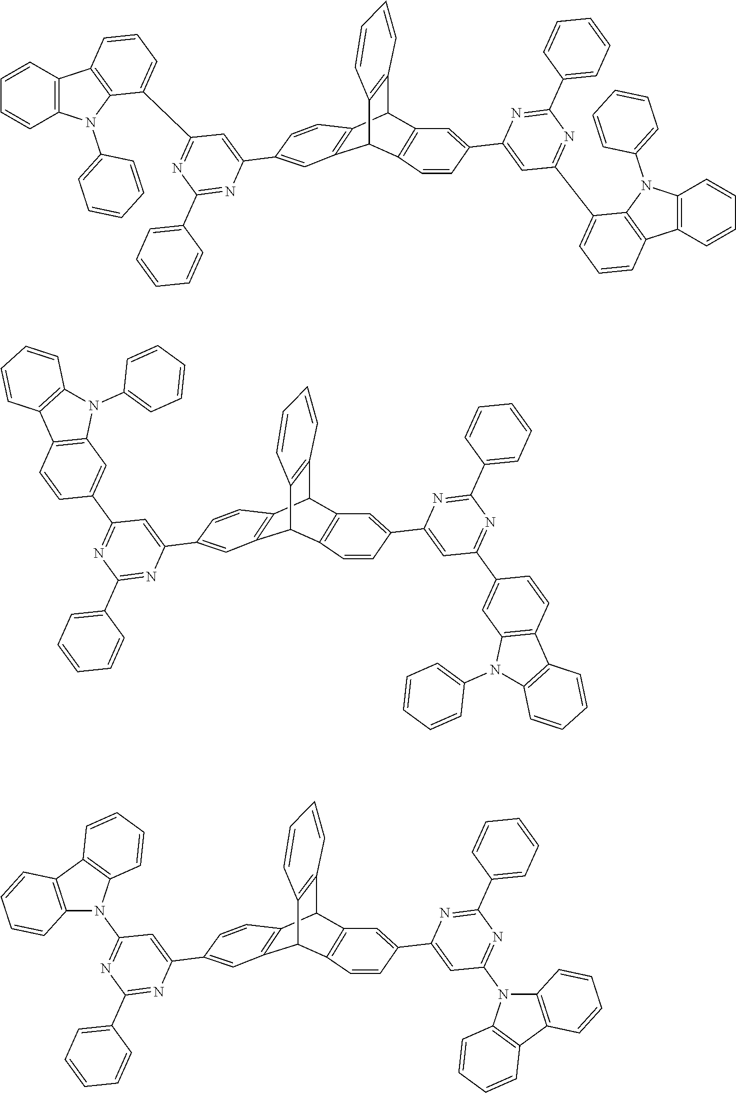 [SMILES: c1ccc(-c2nc(-c3ccc4c(c3)C3c5ccccc5C4c4cc(-c5cc(-c6cccc7c8ccccc8n(-c8ccccc8)c67)nc(-c6ccccc6)n5)ccc43)cc(-c3cccc4c5ccccc5n(-c5ccccc5)c34)n2)cc1 ; c1ccc(-c2nc(-c3ccc4c(c3)C3c5ccccc5C4c4cc(-c5cc(-c6ccc7c8ccccc8n(-c8ccccc8)c7c6)nc(-c6ccccc6)n5)ccc43)cc(-c3ccc4c5ccccc5n(-c5ccccc5)c4c3)n2)cc1 ; c1ccc(-c2nc(-c3ccc4c(c3)C3c5ccccc5C4c4cc(-c5cc(-n6c7ccccc7c7ccccc76)nc(-c6ccccc6)n5)ccc43)cc(-n3c4ccccc4c4ccccc43)n2)cc1]

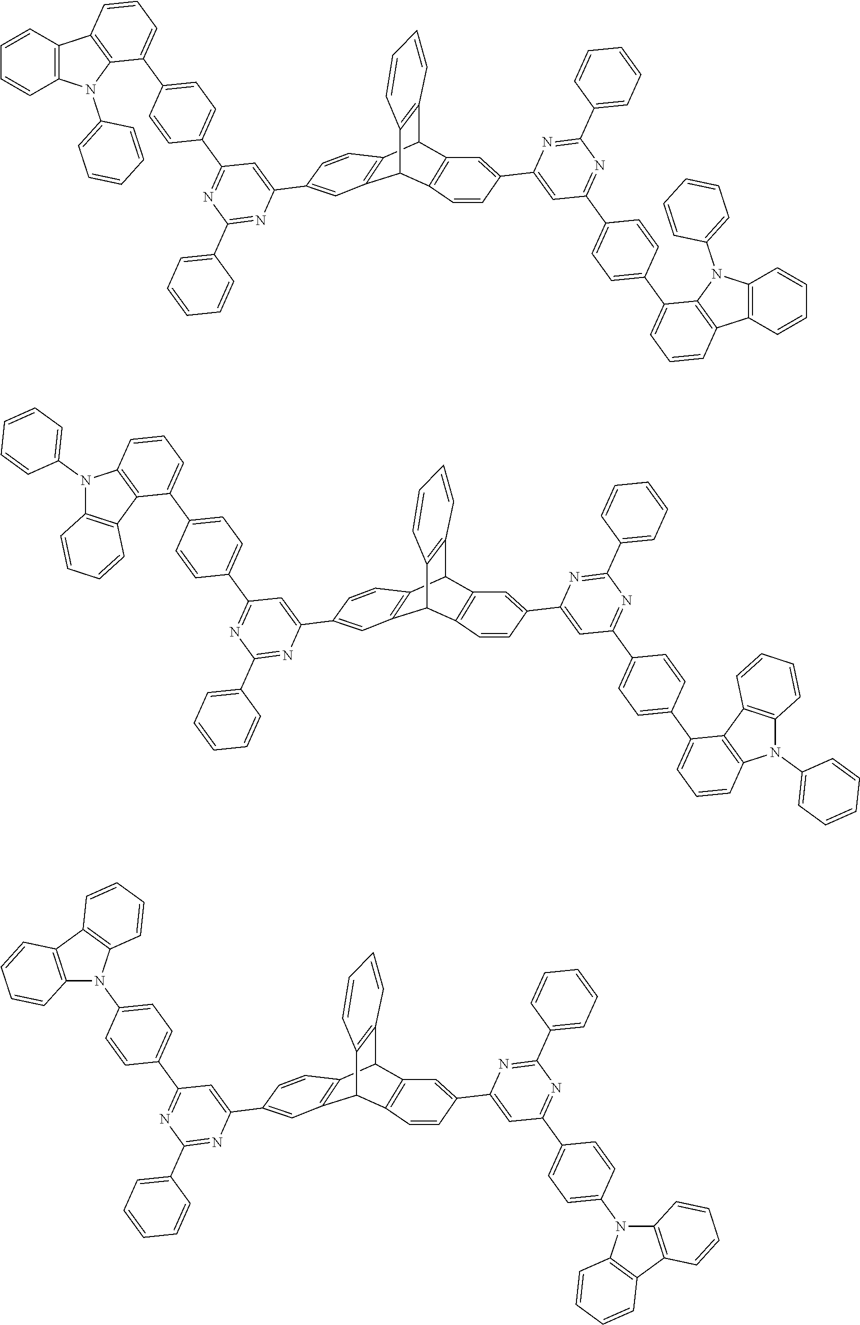 [SMILES: c1ccc(-c2nc(-c3ccc(-c4cccc5c6ccccc6n(-c6ccccc6)c45)cc3)cc(-c3ccc4c(c3)C3c5ccccc5C4c4cc(-c5cc(-c6ccc(-c7cccc8c9ccccc9n(-c9ccccc9)c78)cc6)nc(-c6ccccc6)n5)ccc43)n2)cc1 ; c1ccc(-c2nc(-c3ccc(-c4cccc5c4c4ccccc4n5-c4ccccc4)cc3)cc(-c3ccc4c(c3)C3c5ccccc5C4c4cc(-c5cc(-c6ccc(-c7cccc8c7c7ccccc7n8-c7ccccc7)cc6)nc(-c6ccccc6)n5)ccc43)n2)cc1 ; c1ccc(-c2nc(-c3ccc(-n4c5ccccc5c5ccccc54)cc3)cc(-c3ccc4c(c3)C3c5ccccc5C4c4cc(-c5cc(-c6ccc(-n7c8ccccc8c8ccccc87)cc6)nc(-c6ccccc6)n5)ccc43)n2)cc1]

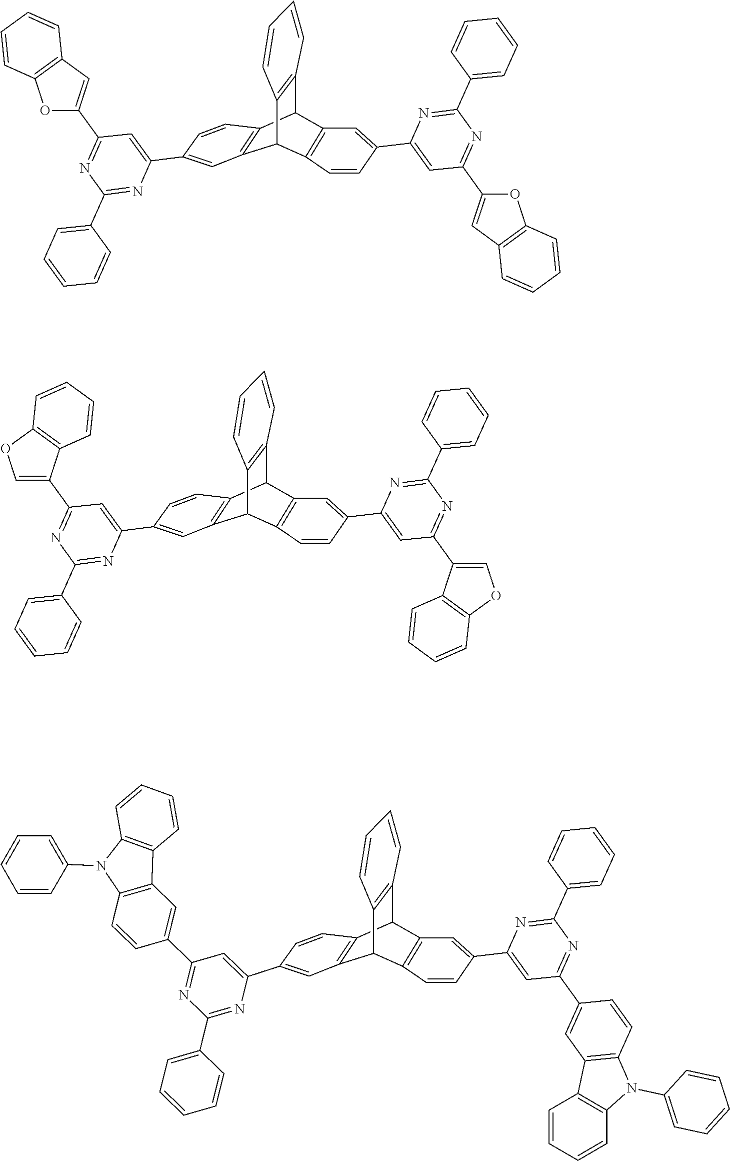 [SMILES: c1ccc(-c2nc(-c3ccc4c(c3)C3c5ccccc5C4c4cc(-c5cc(-c6cc7ccccc7o6)nc(-c6ccccc6)n5)ccc43)cc(-c3cc4ccccc4o3)n2)cc1 ; c1ccc(-c2nc(-c3ccc4c(c3)C3c5ccccc5C4c4cc(-c5cc(-c6coc7ccccc67)nc(-c6ccccc6)n5)ccc43)cc(-c3coc4ccccc34)n2)cc1 ; c1ccc(-c2nc(-c3ccc4c(c3)C3c5ccccc5C4c4cc(-c5cc(-c6ccc7c(c6)c6ccccc6n7-c6ccccc6)nc(-c6ccccc6)n5)ccc43)cc(-c3ccc4c(c3)c3ccccc3n4-c3ccccc3)n2)cc1]

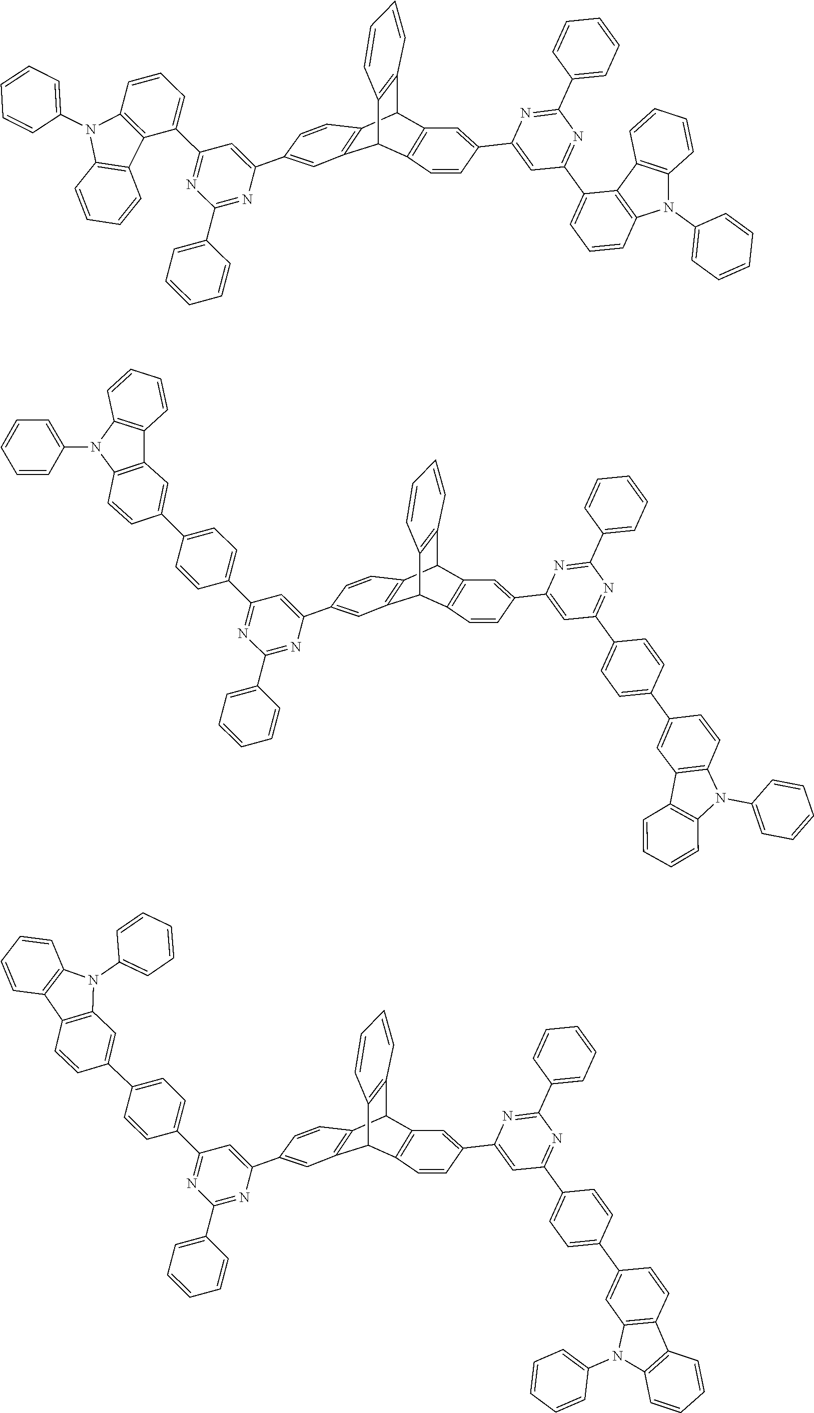 [SMILES: c1ccc(-c2nc(-c3ccc4c(c3)C3c5ccccc5C4c4cc(-c5cc(-c6cccc7c6c6ccccc6n7-c6ccccc6)nc(-c6ccccc6)n5)ccc43)cc(-c3cccc4c3c3ccccc3n4-c3ccccc3)n2)cc1 ; c1ccc(-c2nc(-c3ccc(-c4ccc5c(c4)c4ccccc4n5-c4ccccc4)cc3)cc(-c3ccc4c(c3)C3c5ccccc5C4c4cc(-c5cc(-c6ccc(-c7ccc8c(c7)c7ccccc7n8-c7ccccc7)cc6)nc(-c6ccccc6)n5)ccc43)n2)cc1 ; c1ccc(-c2nc(-c3ccc(-c4ccc5c6ccccc6n(-c6ccccc6)c5c4)cc3)cc(-c3ccc4c(c3)C3c5ccccc5C4c4cc(-c5cc(-c6ccc(-c7ccc8c9ccccc9n(-c9ccccc9)c8c7)cc6)nc(-c6ccccc6)n5)ccc43)n2)cc1]

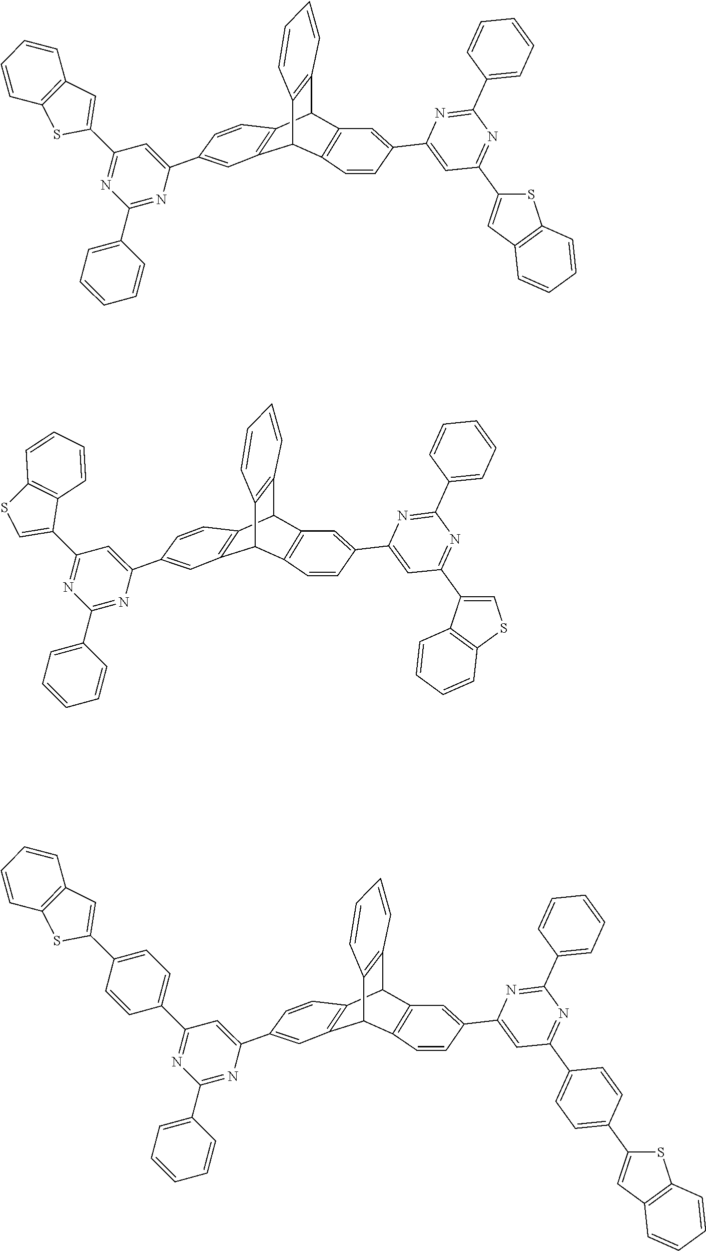 [SMILES: c1ccc(-c2nc(-c3ccc4c(c3)C3c5ccccc5C4c4cc(-c5cc(-c6cc7ccccc7s6)nc(-c6ccccc6)n5)ccc43)cc(-c3cc4ccccc4s3)n2)cc1 ; c1ccc(-c2nc(-c3ccc4c(c3)C3c5ccccc5C4c4cc(-c5cc(-c6csc7ccccc67)nc(-c6ccccc6)n5)ccc43)cc(-c3csc4ccccc34)n2)cc1 ; c1ccc(-c2nc(-c3ccc(-c4cc5ccccc5s4)cc3)cc(-c3ccc4c(c3)C3c5ccccc5C4c4cc(-c5cc(-c6ccc(-c7cc8ccccc8s7)cc6)nc(-c6ccccc6)n5)ccc43)n2)cc1]

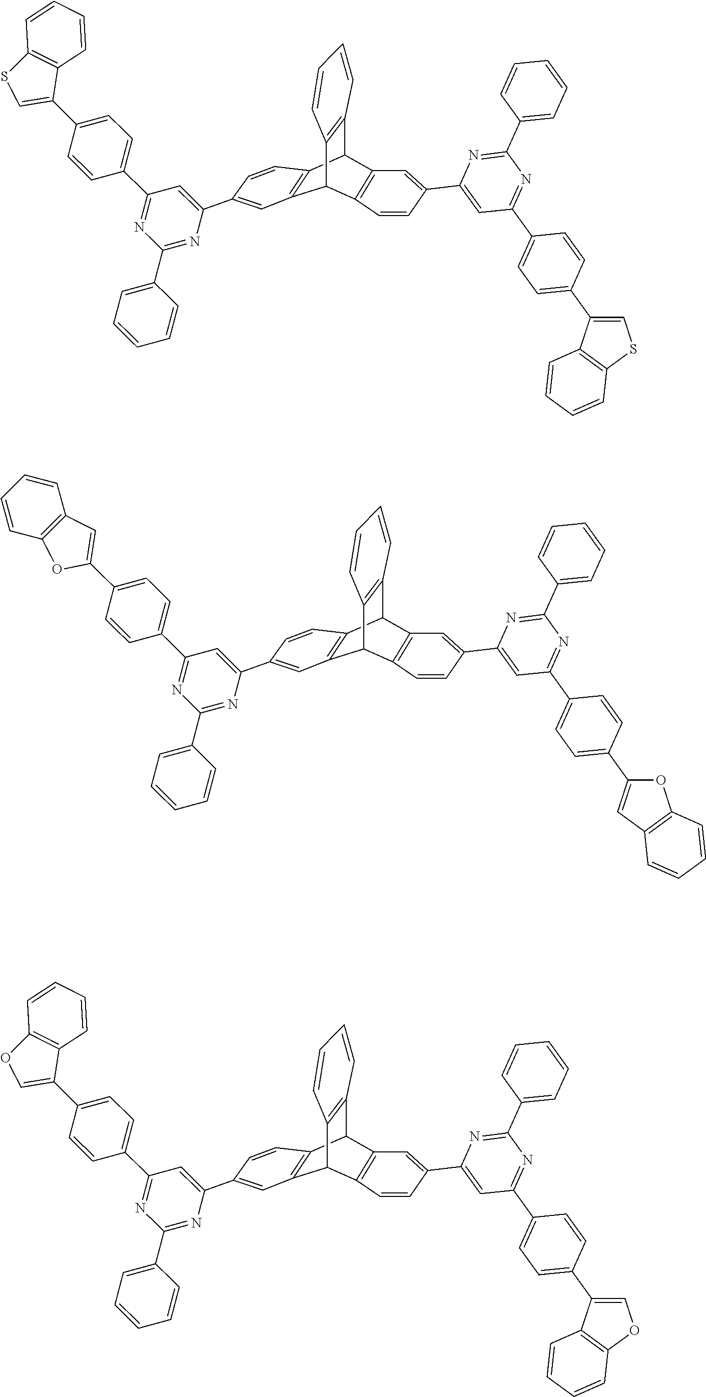 [SMILES: c1ccc(-c2nc(-c3ccc(-c4csc5ccccc45)cc3)cc(-c3ccc4c(c3)C3c5ccccc5C4c4cc(-c5cc(-c6ccc(-c7csc8ccccc78)cc6)nc(-c6ccccc6)n5)ccc43)n2)cc1 ; c1ccc(-c2nc(-c3ccc(-c4cc5ccccc5o4)cc3)cc(-c3ccc4c(c3)C3c5ccccc5C4c4cc(-c5cc(-c6ccc(-c7cc8ccccc8o7)cc6)nc(-c6ccccc6)n5)ccc43)n2)cc1 ; c1ccc(-c2nc(-c3ccc(-c4coc5ccccc45)cc3)cc(-c3ccc4c(c3)C3c5ccccc5C4c4cc(-c5cc(-c6ccc(-c7coc8ccccc78)cc6)nc(-c6ccccc6)n5)ccc43)n2)cc1]

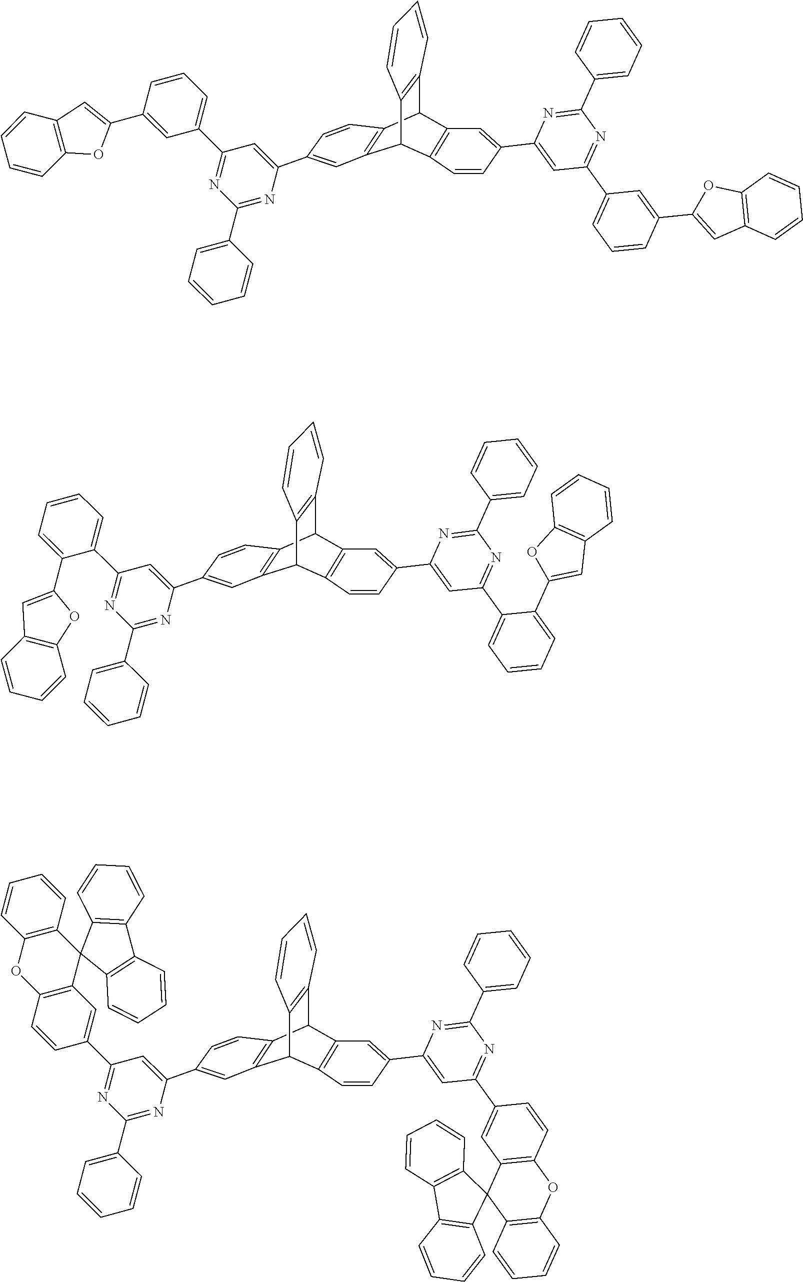 [SMILES: c1ccc(-c2nc(-c3cccc(-c4cc5ccccc5o4)c3)cc(-c3ccc4c(c3)C3c5ccccc5C4c4cc(-c5cc(-c6cccc(-c7cc8ccccc8o7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6cc7ccccc7o6)nc(-c6ccccc6)n5)ccc43)cc(-c3ccccc3-c3cc4ccccc4o3)n2)cc1 ; c1ccc(-c2nc(-c3ccc4c(c3)C3c5ccccc5C4c4cc(-c5cc(-c6ccc7c(c6)C6(c8ccccc8O7)c7ccccc7-c7ccccc76)nc(-c6ccccc6)n5)ccc43)cc(-c3ccc4c(c3)C3(c5ccccc5O4)c4ccccc4-c4ccccc43)n2)cc1]

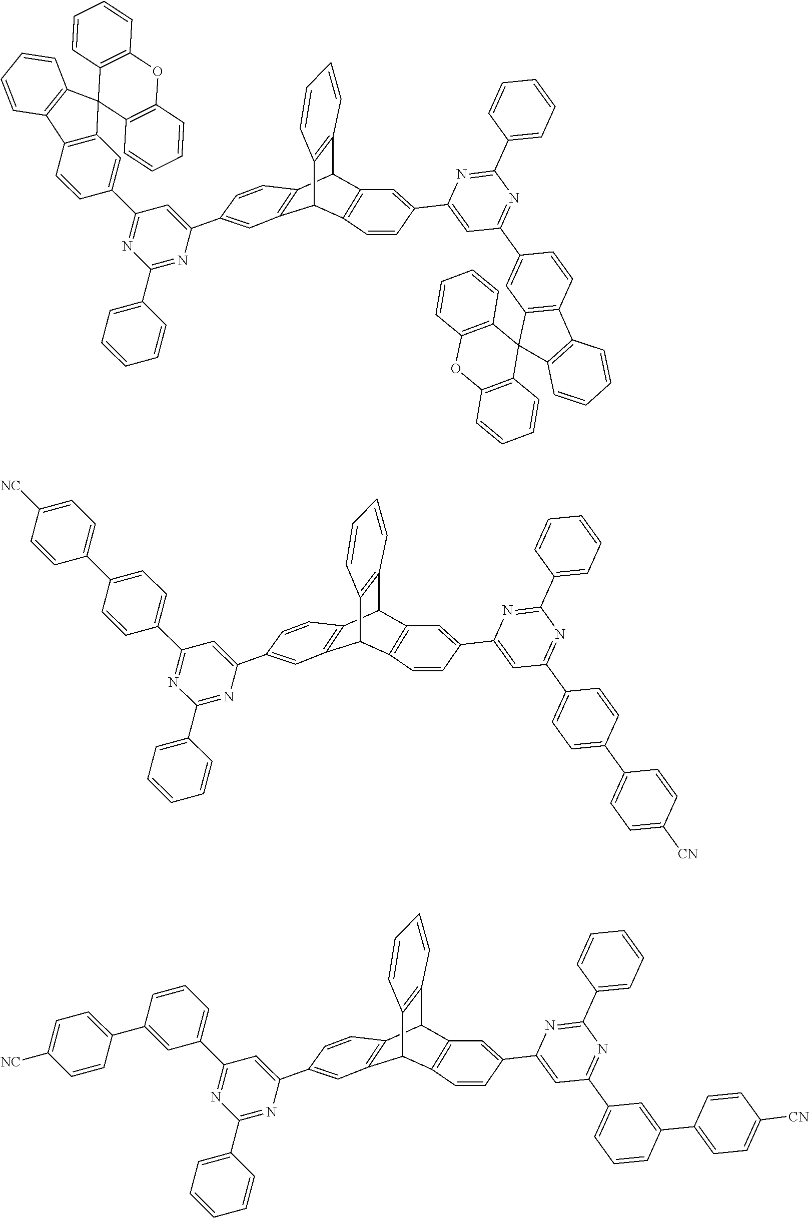 [SMILES: c1ccc(-c2nc(-c3ccc4c(c3)C3c5ccccc5C4c4cc(-c5cc(-c6ccc7c(c6)C6(c8ccccc8Oc8ccccc86)c6ccccc6-7)nc(-c6ccccc6)n5)ccc43)cc(-c3ccc4c(c3)C3(c5ccccc5Oc5ccccc53)c3ccccc3-4)n2)cc1 ; N#Cc1ccc(-c2ccc(-c3cc(-c4ccc5c(c4)C4c6ccccc6C5c5cc(-c6cc(-c7ccc(-c8ccc(C#N)cc8)cc7)nc(-c7ccccc7)n6)ccc54)nc(-c4ccccc4)n3)cc2)cc1 ; N#Cc1ccc(-c2cccc(-c3cc(-c4ccc5c(c4)C4c6ccccc6C5c5cc(-c6cc(-c7cccc(-c8ccc(C#N)cc8)c7)nc(-c7ccccc7)n6)ccc54)nc(-c4ccccc4)n3)c2)cc1]

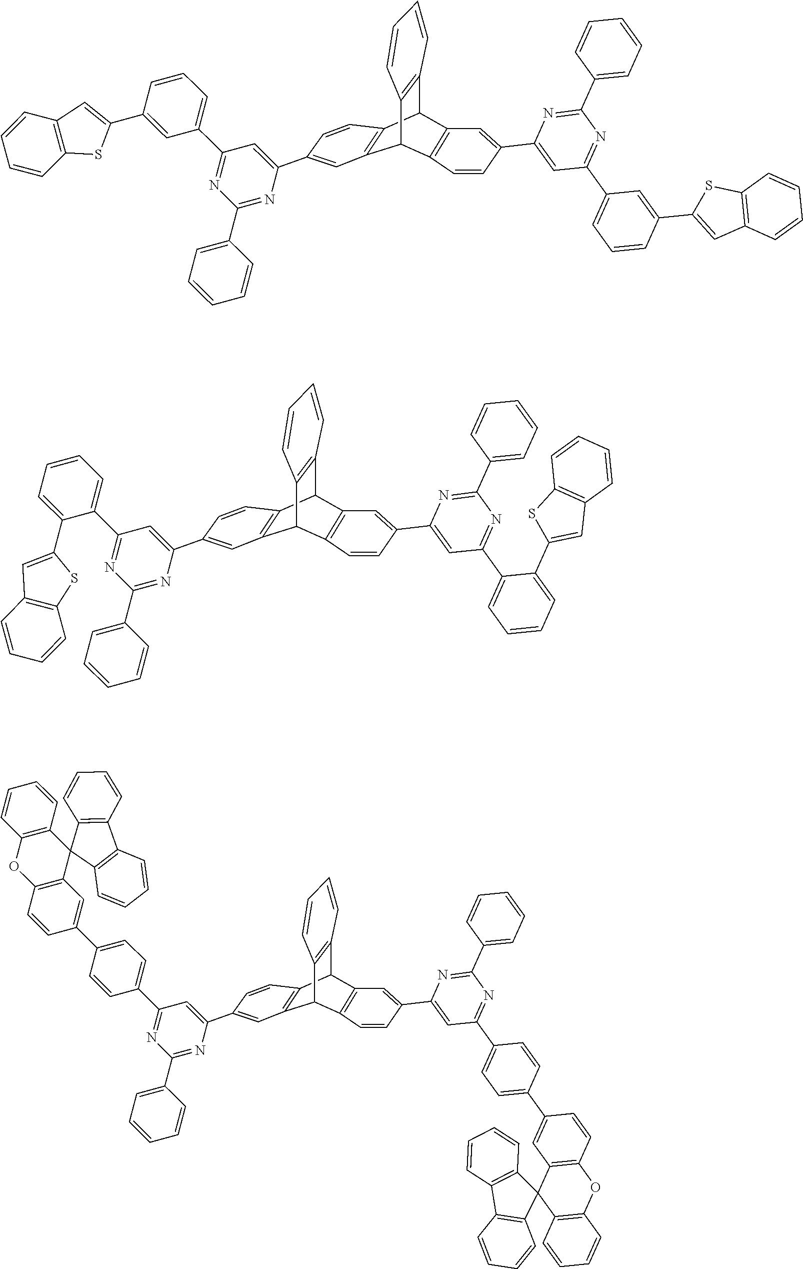 [SMILES: c1ccc(-c2nc(-c3cccc(-c4cc5ccccc5s4)c3)cc(-c3ccc4c(c3)C3c5ccccc5C4c4cc(-c5cc(-c6cccc(-c7cc8ccccc8s7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6cc7ccccc7s6)nc(-c6ccccc6)n5)ccc43)cc(-c3ccccc3-c3cc4ccccc4s3)n2)cc1 ; c1ccc(-c2nc(-c3ccc(-c4ccc5c(c4)C4(c6ccccc6O5)c5ccccc5-c5ccccc54)cc3)cc(-c3ccc4c(c3)C3c5ccccc5C4c4cc(-c5cc(-c6ccc(-c7ccc8c(c7)C7(c9ccccc9O8)c8ccccc8-c8ccccc87)cc6)nc(-c6ccccc6)n5)ccc43)n2)cc1]

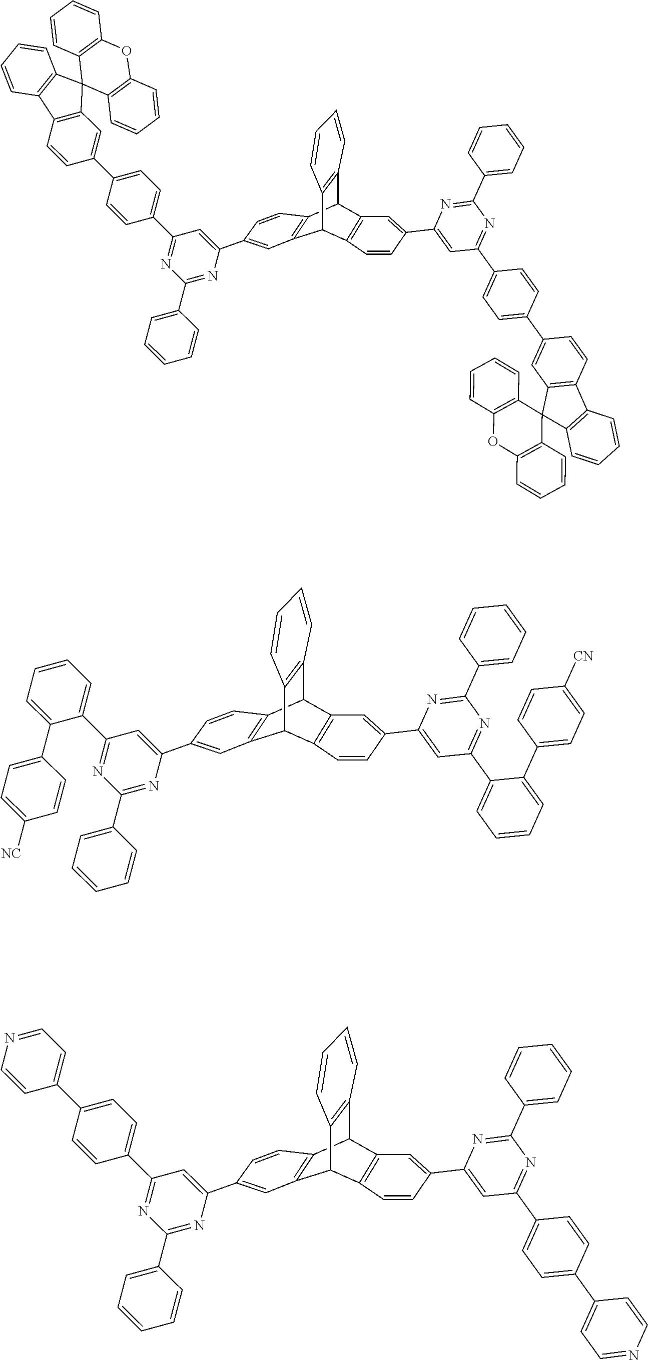 [SMILES: c1ccc(-c2nc(-c3ccc(-c4ccc5c(c4)C4(c6ccccc6Oc6ccccc64)c4ccccc4-5)cc3)cc(-c3ccc4c(c3)C3c5ccccc5C4c4cc(-c5cc(-c6ccc(-c7ccc8c(c7)C7(c9ccccc9Oc9ccccc97)c7ccccc7-8)cc6)nc(-c6ccccc6)n5)ccc43)n2)cc1 ; N#Cc1ccc(-c2ccccc2-c2cc(-c3ccc4c(c3)C3c5ccccc5C4c4cc(-c5cc(-c6ccccc6-c6ccc(C#N)cc6)nc(-c6ccccc6)n5)ccc43)nc(-c3ccccc3)n2)cc1 ; c1ccc(-c2nc(-c3ccc(-c4ccncc4)cc3)cc(-c3ccc4c(c3)C3c5ccccc5C4c4cc(-c5cc(-c6ccc(-c7ccncc7)cc6)nc(-c6ccccc6)n5)ccc43)n2)cc1]

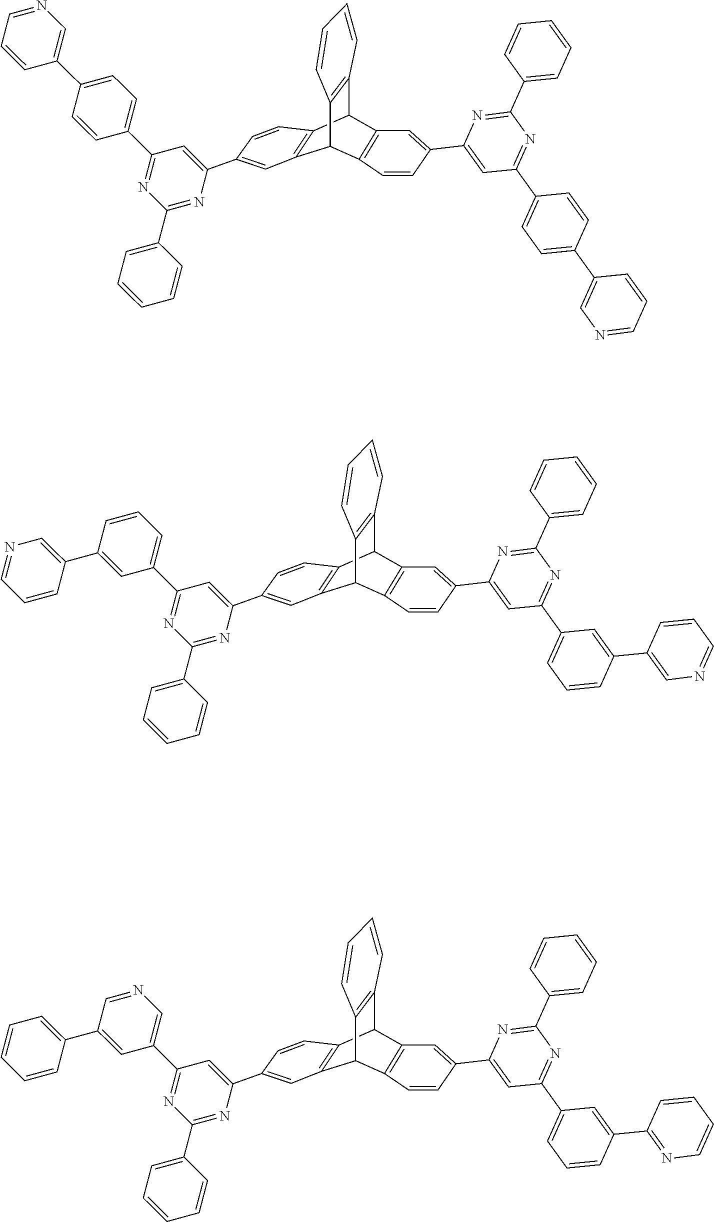 [SMILES: c1ccc(-c2nc(-c3ccc(-c4cccnc4)cc3)cc(-c3ccc4c(c3)C3c5ccccc5C4c4cc(-c5cc(-c6ccc(-c7cccnc7)cc6)nc(-c6ccccc6)n5)ccc43)n2)cc1 ; c1ccc(-c2nc(-c3cccc(-c4cccnc4)c3)cc(-c3ccc4c(c3)C3c5ccccc5C4c4cc(-c5cc(-c6cccc(-c7cccnc7)c6)nc(-c6ccccc6)n5)ccc43)n2)cc1 ; c1ccc(-c2cncc(-c3cc(-c4ccc5c(c4)C4c6ccccc6C5c5cc(-c6cc(-c7cccc(-c8ccccn8)c7)nc(-c7ccccc7)n6)ccc54)nc(-c4ccccc4)n3)c2)cc1]

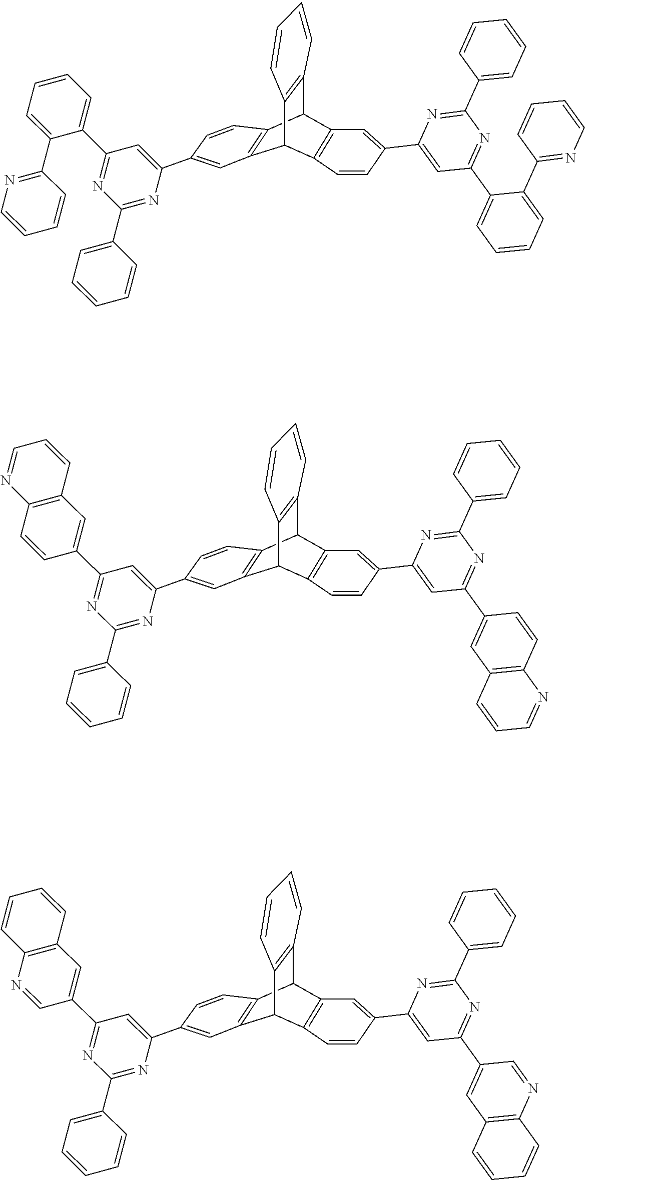 [SMILES: c1ccc(-c2nc(-c3ccc4c(c3)C3c5ccccc5C4c4cc(-c5cc(-c6ccccc6-c6ccccn6)nc(-c6ccccc6)n5)ccc43)cc(-c3ccccc3-c3ccccn3)n2)cc1 ; c1ccc(-c2nc(-c3ccc4c(c3)C3c5ccccc5C4c4cc(-c5cc(-c6ccc7ncccc7c6)nc(-c6ccccc6)n5)ccc43)cc(-c3ccc4ncccc4c3)n2)cc1 ; c1ccc(-c2nc(-c3ccc4c(c3)C3c5ccccc5C4c4cc(-c5cc(-c6cnc7ccccc7c6)nc(-c6ccccc6)n5)ccc43)cc(-c3cnc4ccccc4c3)n2)cc1]

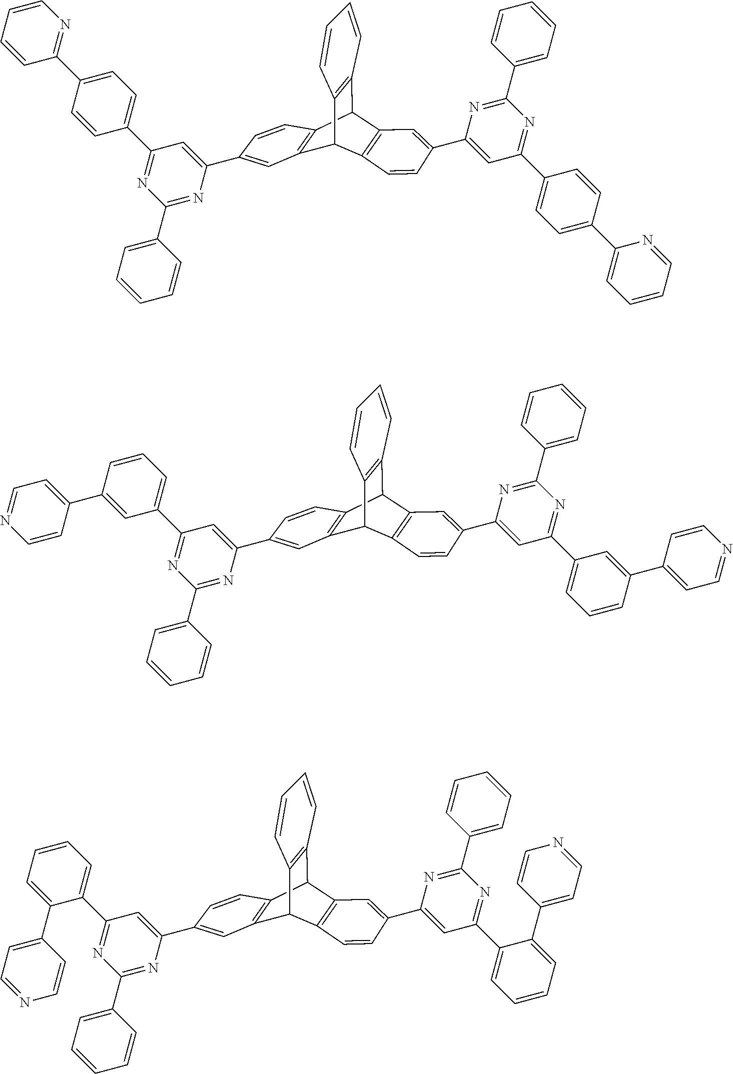 [SMILES: c1ccc(-c2nc(-c3ccc(-c4ccccn4)cc3)cc(-c3ccc4c(c3)C3c5ccccc5C4c4cc(-c5cc(-c6ccc(-c7ccccn7)cc6)nc(-c6ccccc6)n5)ccc43)n2)cc1 ; c1ccc(-c2nc(-c3cccc(-c4ccncc4)c3)cc(-c3ccc4c(c3)C3c5ccccc5C4c4cc(-c5cc(-c6cccc(-c7ccncc7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6ccncc6)nc(-c6ccccc6)n5)ccc43)cc(-c3ccccc3-c3ccncc3)n2)cc1]

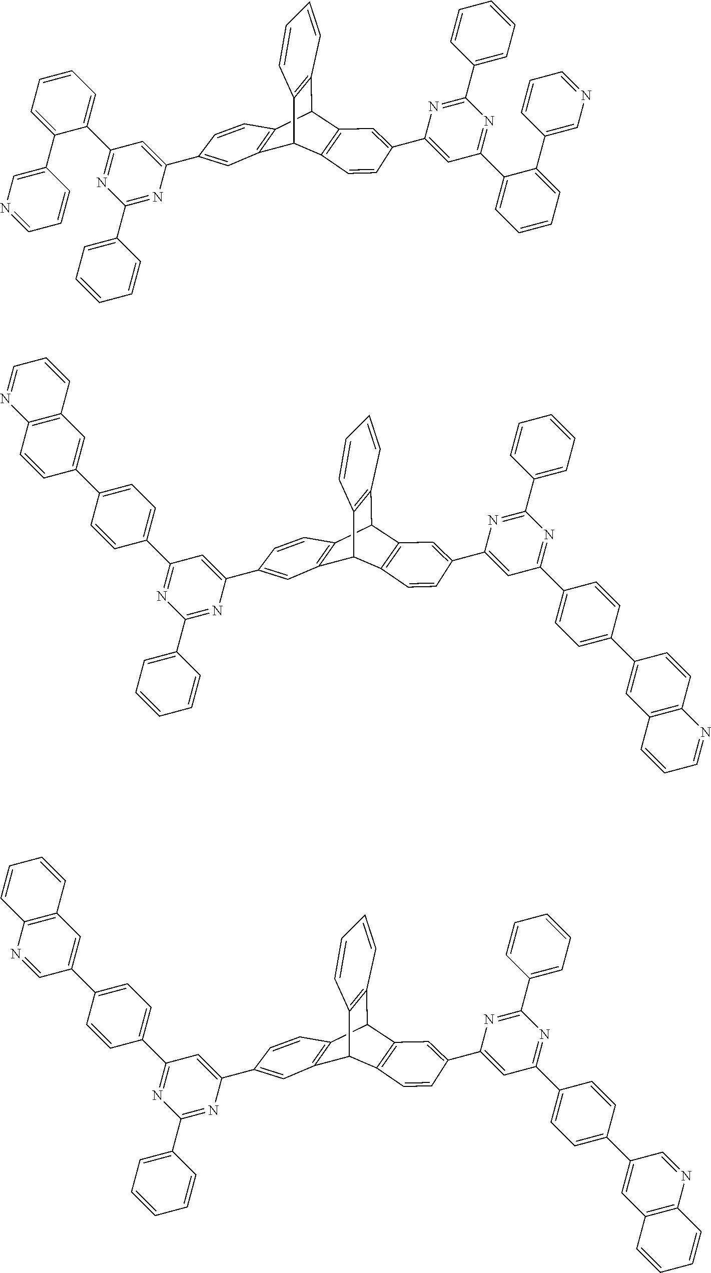 [SMILES: c1ccc(-c2nc(-c3ccc4c(c3)C3c5ccccc5C4c4cc(-c5cc(-c6ccccc6-c6cccnc6)nc(-c6ccccc6)n5)ccc43)cc(-c3ccccc3-c3cccnc3)n2)cc1 ; c1ccc(-c2nc(-c3ccc(-c4ccc5ncccc5c4)cc3)cc(-c3ccc4c(c3)C3c5ccccc5C4c4cc(-c5cc(-c6ccc(-c7ccc8ncccc8c7)cc6)nc(-c6ccccc6)n5)ccc43)n2)cc1 ; c1ccc(-c2nc(-c3ccc(-c4cnc5ccccc5c4)cc3)cc(-c3ccc4c(c3)C3c5ccccc5C4c4cc(-c5cc(-c6ccc(-c7cnc8ccccc8c7)cc6)nc(-c6ccccc6)n5)ccc43)n2)cc1]

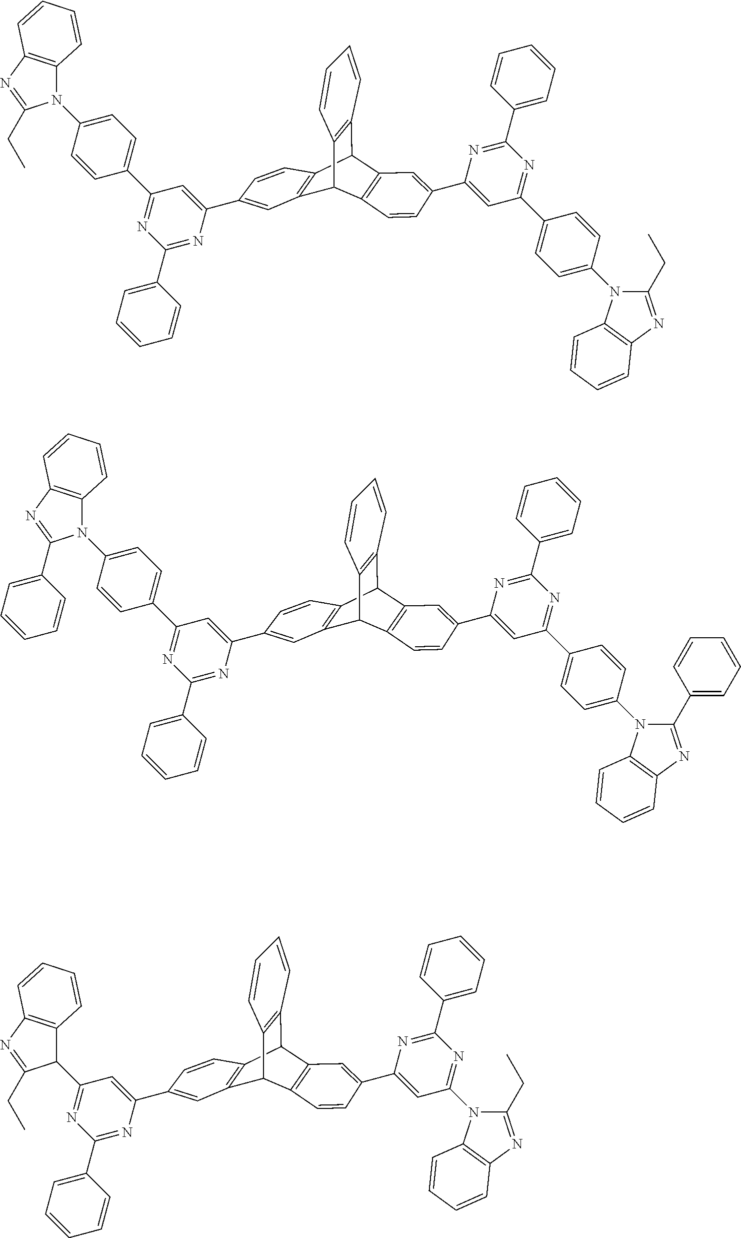 [SMILES: CCc1nc2ccccc2n1-c1ccc(-c2cc(-c3ccc4c(c3)C3c5ccccc5C4c4cc(-c5cc(-c6ccc(-n7c(CC)nc8ccccc87)cc6)nc(-c6ccccc6)n5)ccc43)nc(-c3ccccc3)n2)cc1 ; c1ccc(-c2nc(-c3ccc(-n4c(-c5ccccc5)nc5ccccc54)cc3)cc(-c3ccc4c(c3)C3c5ccccc5C4c4cc(-c5cc(-c6ccc(-n7c(-c8ccccc8)nc8ccccc87)cc6)nc(-c6ccccc6)n5)ccc43)n2)cc1 ; CCC1=Nc2ccccc2C1c1cc(-c2ccc3c(c2)C2c4ccccc4C3c3cc(-c4cc(-n5c(CC)nc6ccccc65)nc(-c5ccccc5)n4)ccc32)nc(-c2ccccc2)n1]

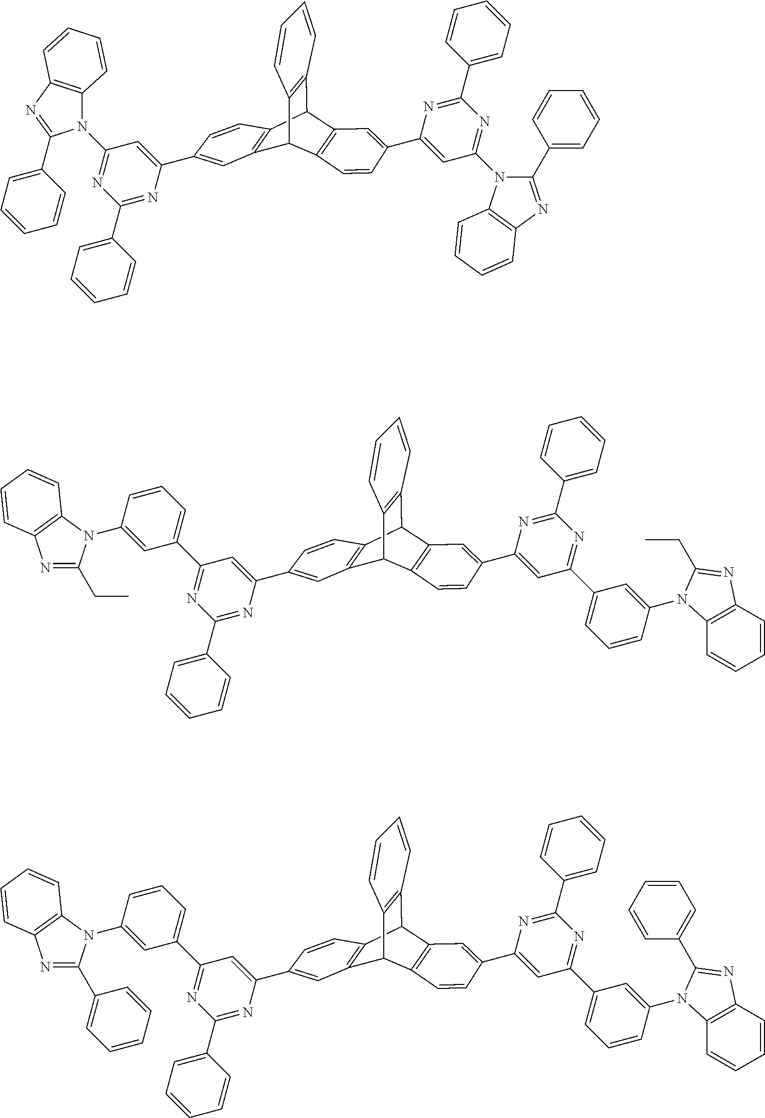 [SMILES: c1ccc(-c2nc(-c3ccc4c(c3)C3c5ccccc5C4c4cc(-c5cc(-n6c(-c7ccccc7)nc7ccccc76)nc(-c6ccccc6)n5)ccc43)cc(-n3c(-c4ccccc4)nc4ccccc43)n2)cc1 ; CCc1nc2ccccc2n1-c1cccc(-c2cc(-c3ccc4c(c3)C3c5ccccc5C4c4cc(-c5cc(-c6cccc(-n7c(CC)nc8ccccc87)c6)nc(-c6ccccc6)n5)ccc43)nc(-c3ccccc3)n2)c1 ; c1ccc(-c2nc(-c3cccc(-n4c(-c5ccccc5)nc5ccccc54)c3)cc(-c3ccc4c(c3)C3c5ccccc5C4c4cc(-c5cc(-c6cccc(-n7c(-c8ccccc8)nc8ccccc87)c6)nc(-c6ccccc6)n5)ccc43)n2)cc1]

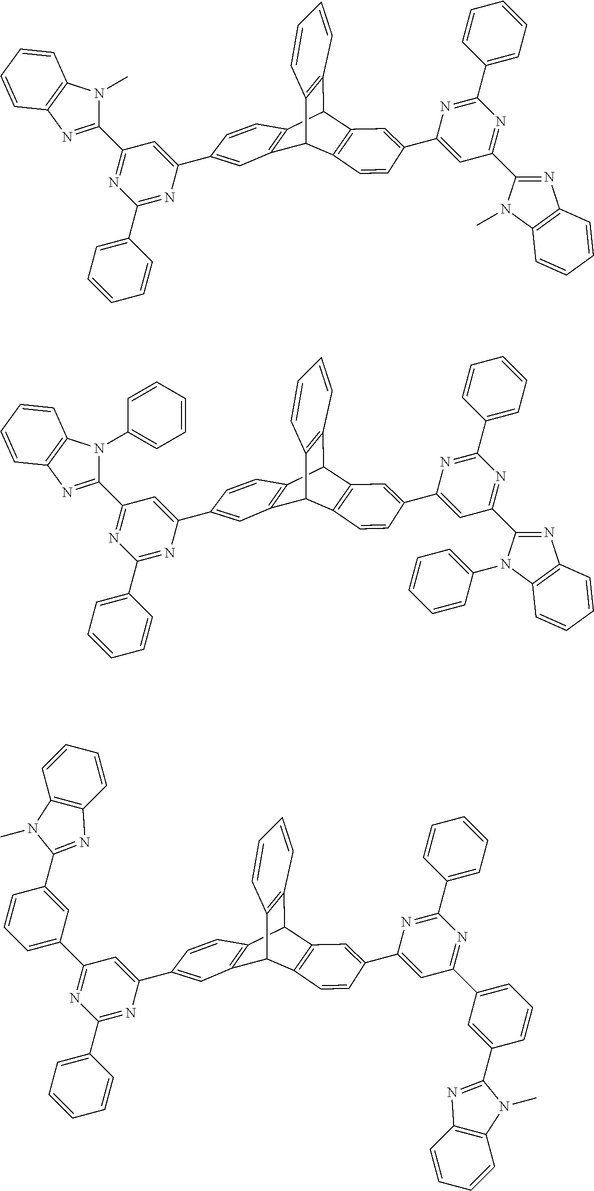

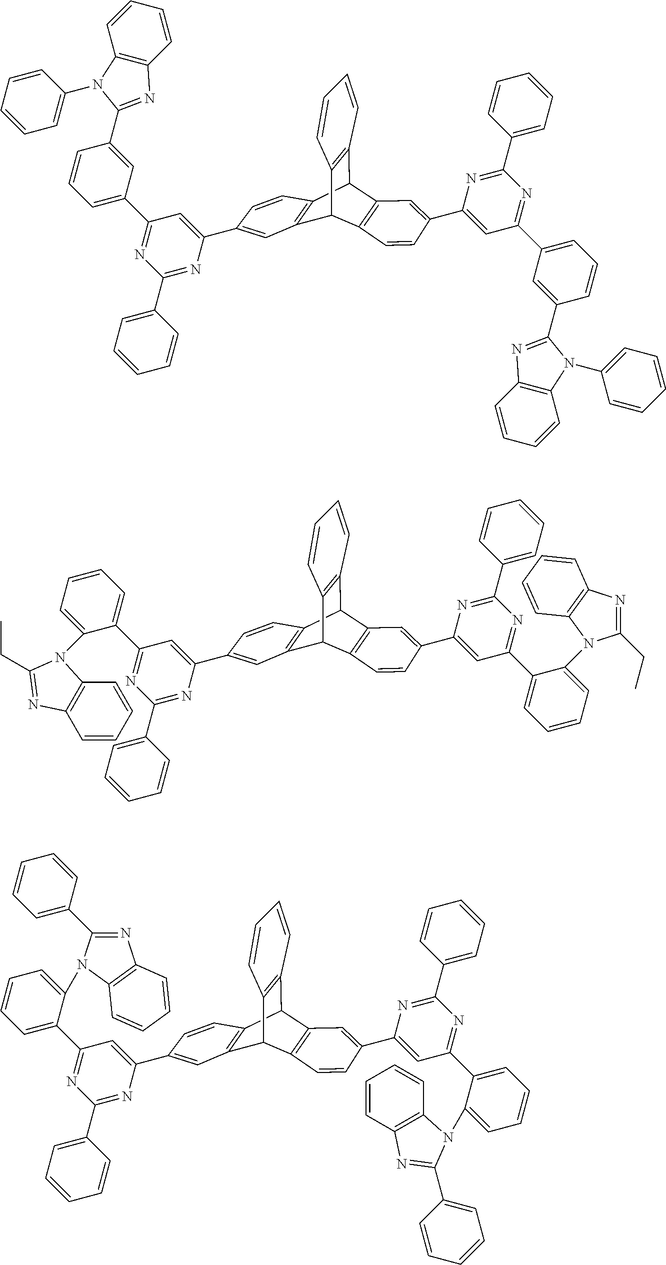 [SMILES: c1ccc(-c2nc(-c3cccc(-c4nc5ccccc5n4-c4ccccc4)c3)cc(-c3ccc4c(c3)C3c5ccccc5C4c4cc(-c5cc(-c6cccc(-c7nc8ccccc8n7-c7ccccc7)c6)nc(-c6ccccc6)n5)ccc43)n2)cc1 ; CCc1nc2ccccc2n1-c1ccccc1-c1cc(-c2ccc3c(c2)C2c4ccccc4C3c3cc(-c4cc(-c5ccccc5-n5c(CC)nc6ccccc65)nc(-c5ccccc5)n4)ccc32)nc(-c2ccccc2)n1 ; c1ccc(-c2nc(-c3ccc4c(c3)C3c5ccccc5C4c4cc(-c5cc(-c6ccccc6-n6c(-c7ccccc7)nc7ccccc76)nc(-c6ccccc6)n5)ccc43)cc(-c3ccccc3-n3c(-c4ccccc4)nc4ccccc43)n2)cc1]

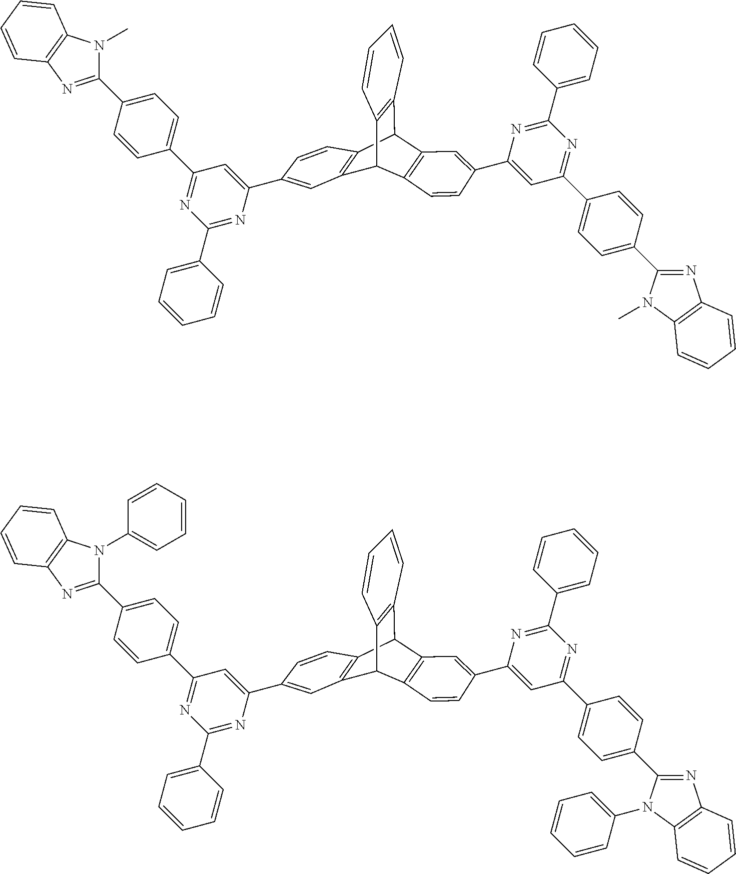 [SMILES: Cn1c(-c2ccc(-c3cc(-c4ccc5c(c4)C4c6ccccc6C5c5cc(-c6cc(-c7ccc(-c8nc9ccccc9n8C)cc7)nc(-c7ccccc7)n6)ccc54)nc(-c4ccccc4)n3)cc2)nc2ccccc21 ; c1ccc(-c2nc(-c3ccc(-c4nc5ccccc5n4-c4ccccc4)cc3)cc(-c3ccc4c(c3)C3c5ccccc5C4c4cc(-c5cc(-c6ccc(-c7nc8ccccc8n7-c7ccccc7)cc6)nc(-c6ccccc6)n5)ccc43)n2)cc1]

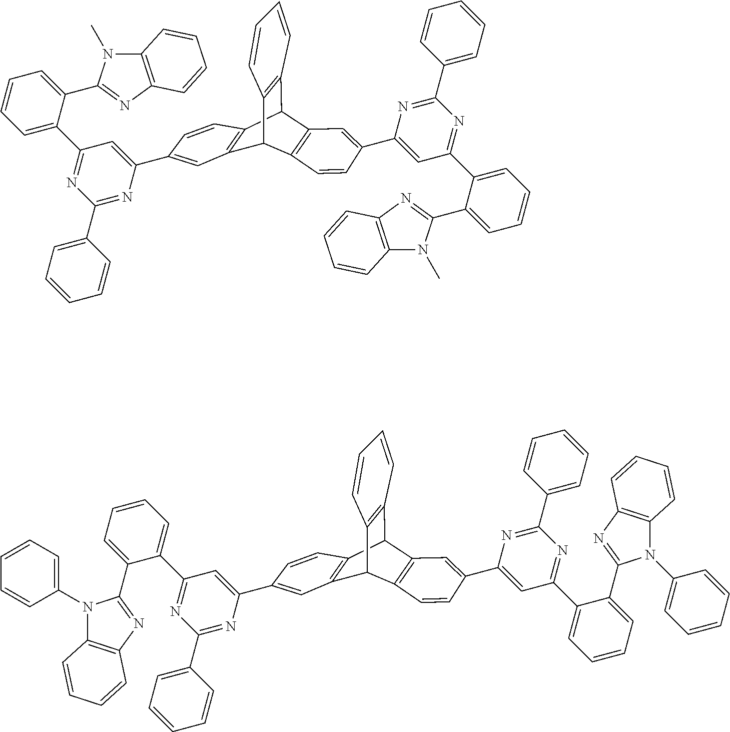 [SMILES: Cn1c(-c2ccccc2-c2cc(-c3ccc4c(c3)C3c5ccccc5C4c4cc(-c5cc(-c6ccccc6-c6nc7ccccc7n6C)nc(-c6ccccc6)n5)ccc43)nc(-c3ccccc3)n2)nc2ccccc21 ; c1ccc(-c2nc(-c3ccc4c(c3)C3c5ccccc5C4c4cc(-c5cc(-c6ccccc6-c6nc7ccccc7n6-c6ccccc6)nc(-c6ccccc6)n5)ccc43)cc(-c3ccccc3-c3nc4ccccc4n3-c3ccccc3)n2)cc1]

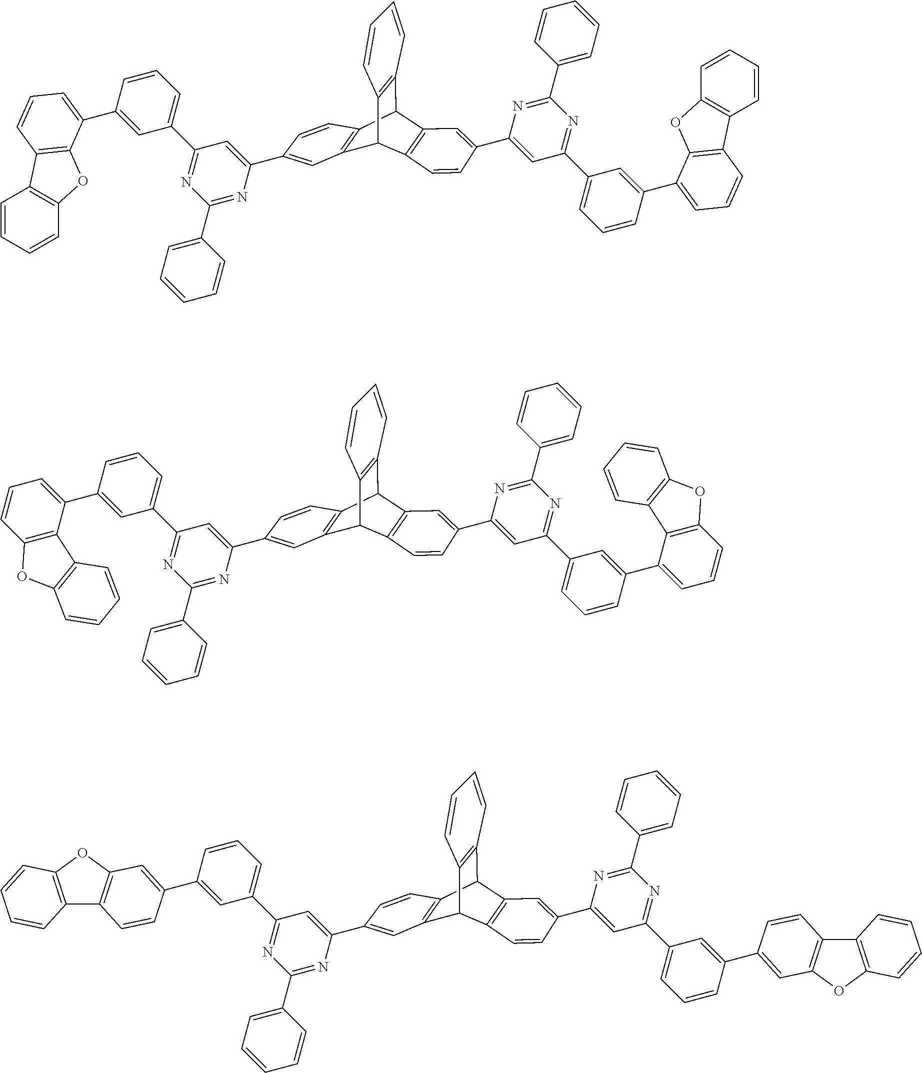 [SMILES: c1ccc(-c2nc(-c3cccc(-c4cccc5c4oc4ccccc45)c3)cc(-c3ccc4c(c3)C3c5ccccc5C4c4cc(-c5cc(-c6cccc(-c7cccc8c7oc7ccccc78)c6)nc(-c6ccccc6)n5)ccc43)n2)cc1 ; c1ccc(-c2nc(-c3cccc(-c4cccc5oc6ccccc6c45)c3)cc(-c3ccc4c(c3)C3c5ccccc5C4c4cc(-c5cc(-c6cccc(-c7cccc8oc9ccccc9c78)c6)nc(-c6ccccc6)n5)ccc43)n2)cc1 ; c1ccc(-c2nc(-c3cccc(-c4ccc5c(c4)oc4ccccc45)c3)cc(-c3ccc4c(c3)C3c5ccccc5C4c4cc(-c5cc(-c6cccc(-c7ccc8c(c7)oc7ccccc78)c6)nc(-c6ccccc6)n5)ccc43)n2)cc1]

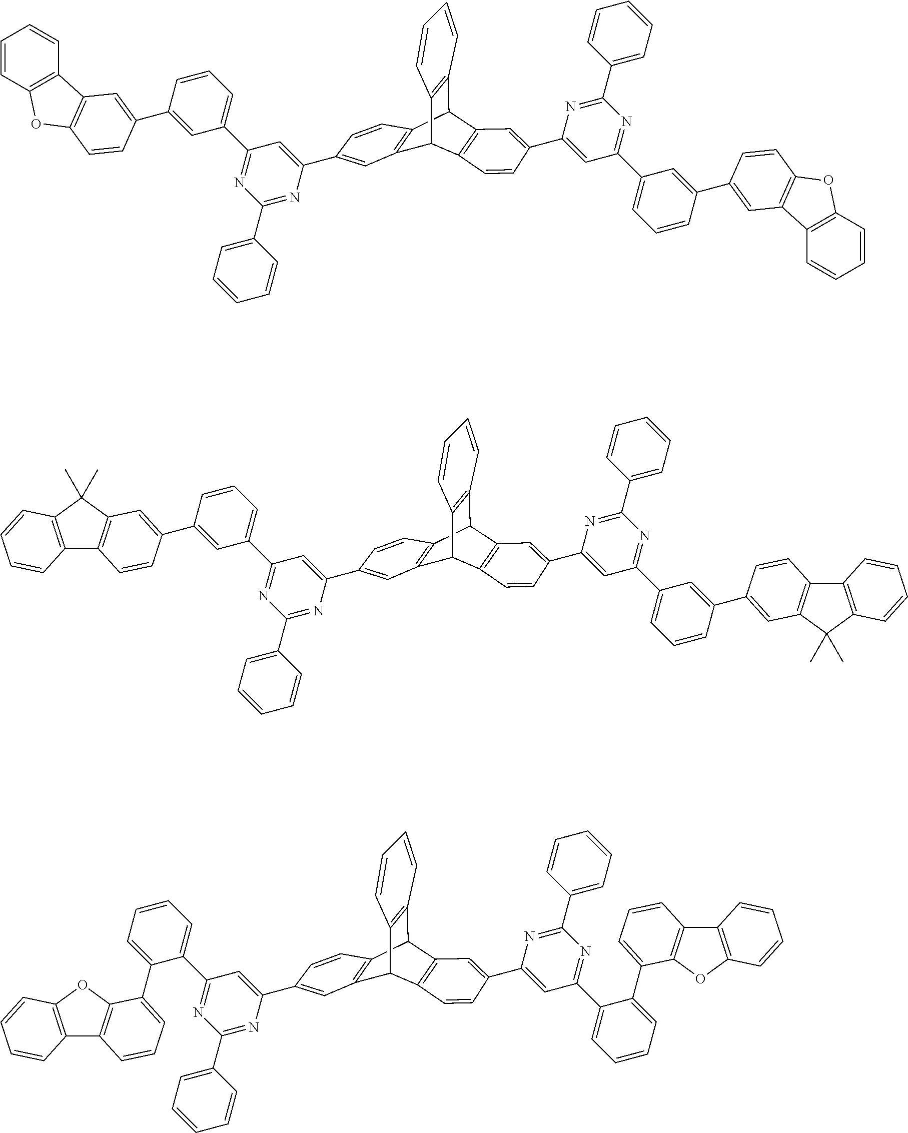 [SMILES: c1ccc(-c2nc(-c3cccc(-c4ccc5oc6ccccc6c5c4)c3)cc(-c3ccc4c(c3)C3c5ccccc5C4c4cc(-c5cc(-c6cccc(-c7ccc8oc9ccccc9c8c7)c6)nc(-c6ccccc6)n5)ccc43)n2)cc1 ; CC1(C)c2ccccc2-c2ccc(-c3cccc(-c4cc(-c5ccc6c(c5)C5c7ccccc7C6c6cc(-c7cc(-c8cccc(-c9ccc%10c(c9)C(C)(C)c9ccccc9-%10)c8)nc(-c8ccccc8)n7)ccc65)nc(-c5ccccc5)n4)c3)cc21 ; c1ccc(-c2nc(-c3ccc4c(c3)C3c5ccccc5C4c4cc(-c5cc(-c6ccccc6-c6cccc7c6oc6ccccc67)nc(-c6ccccc6)n5)ccc43)cc(-c3ccccc3-c3cccc4c3oc3ccccc34)n2)cc1]

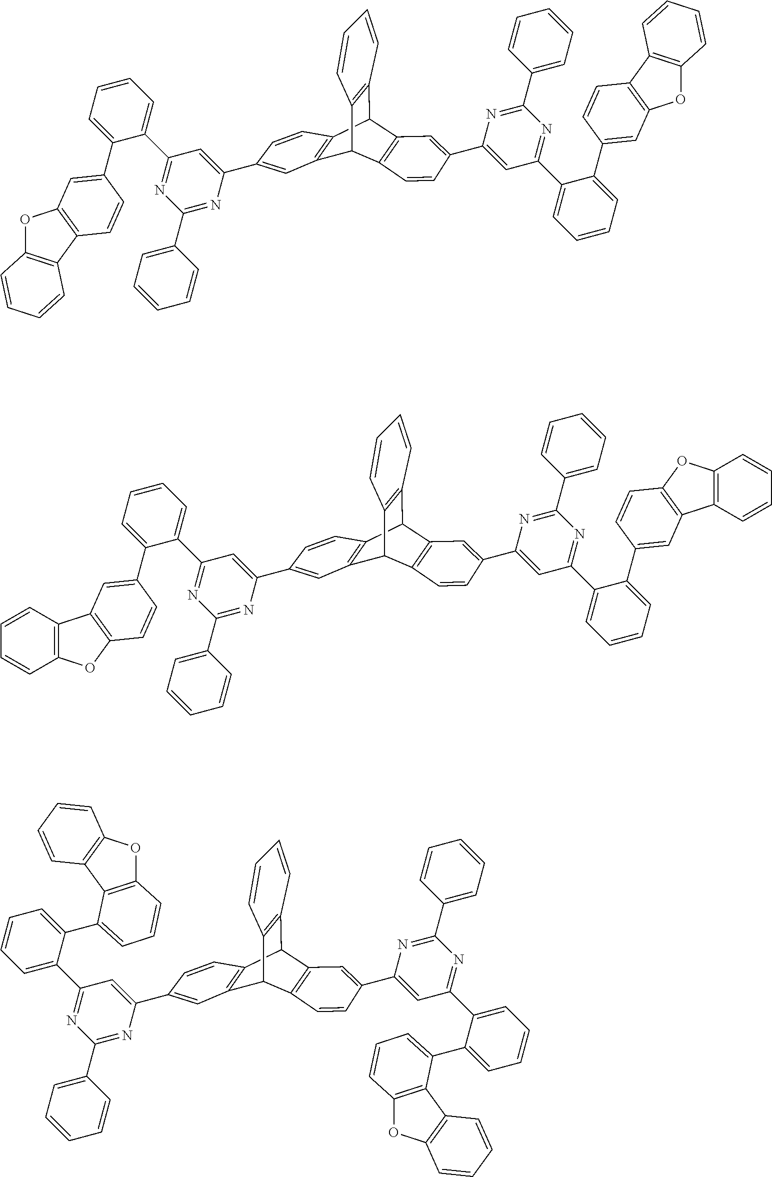 [SMILES: c1ccc(-c2nc(-c3ccc4c(c3)C3c5ccccc5C4c4cc(-c5cc(-c6ccccc6-c6ccc7c(c6)oc6ccccc67)nc(-c6ccccc6)n5)ccc43)cc(-c3ccccc3-c3ccc4c(c3)oc3ccccc34)n2)cc1 ; c1ccc(-c2nc(-c3ccc4c(c3)C3c5ccccc5C4c4cc(-c5cc(-c6ccccc6-c6ccc7oc8ccccc8c7c6)nc(-c6ccccc6)n5)ccc43)cc(-c3ccccc3-c3ccc4oc5ccccc5c4c3)n2)cc1 ; c1ccc(-c2nc(-c3ccc4c(c3)C3c5ccccc5C4c4cc(-c5cc(-c6ccccc6-c6cccc7oc8ccccc8c67)nc(-c6ccccc6)n5)ccc43)cc(-c3ccccc3-c3cccc4oc5ccccc5c34)n2)cc1]

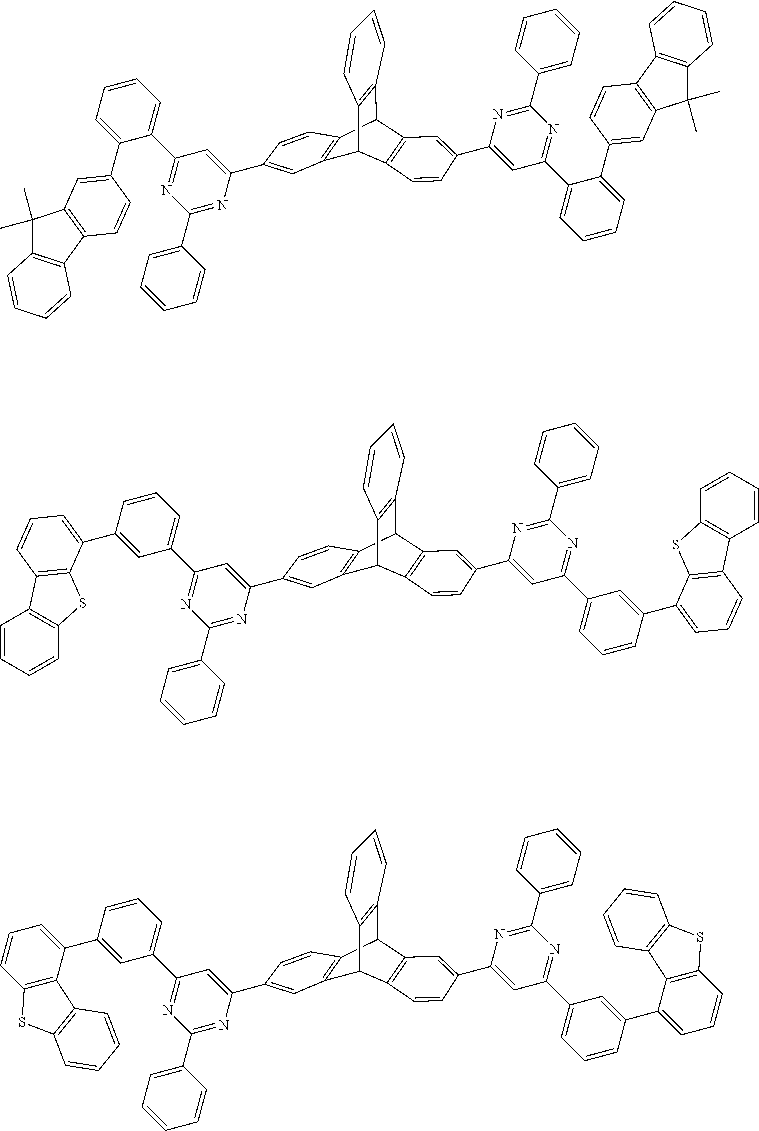

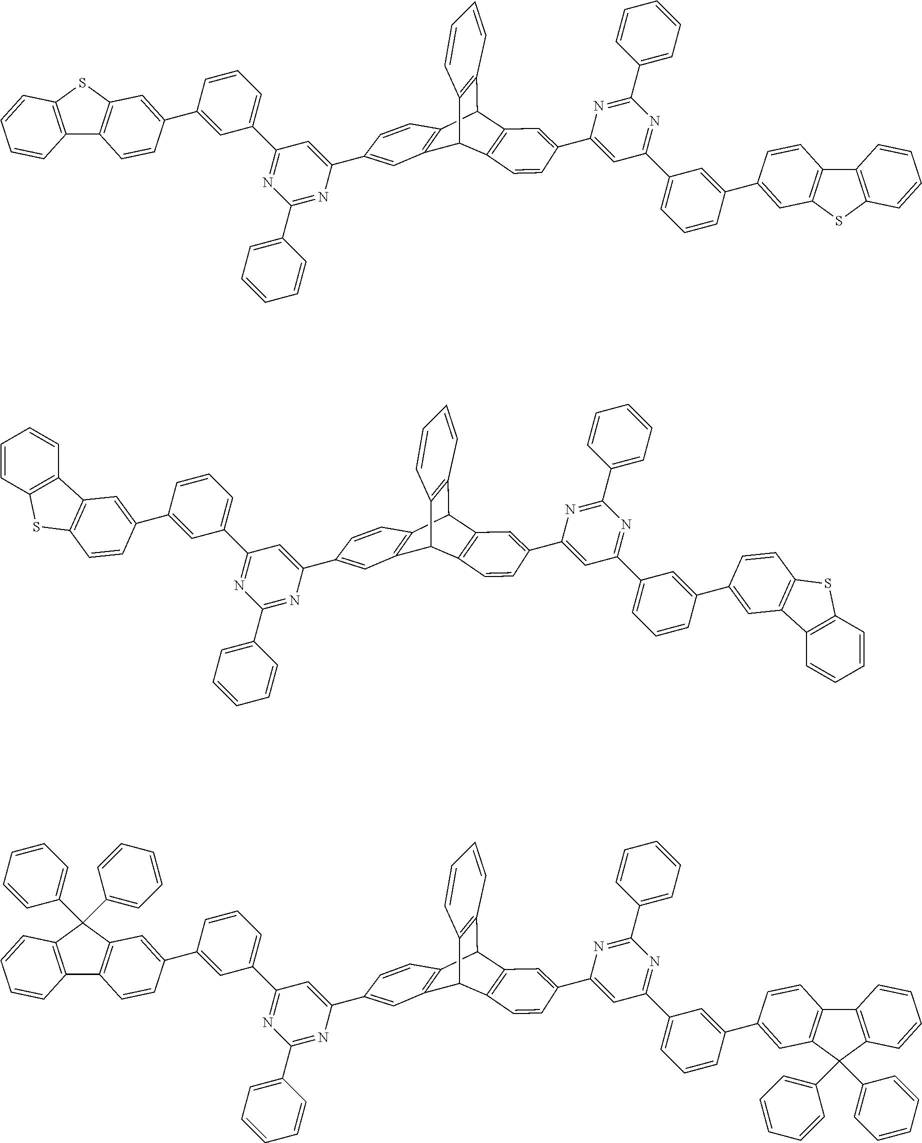 [SMILES: c1ccc(-c2nc(-c3cccc(-c4ccc5c(c4)sc4ccccc45)c3)cc(-c3ccc4c(c3)C3c5ccccc5C4c4cc(-c5cc(-c6cccc(-c7ccc8c(c7)sc7ccccc78)c6)nc(-c6ccccc6)n5)ccc43)n2)cc1 ; c1ccc(-c2nc(-c3cccc(-c4ccc5sc6ccccc6c5c4)c3)cc(-c3ccc4c(c3)C3c5ccccc5C4c4cc(-c5cc(-c6cccc(-c7ccc8sc9ccccc9c8c7)c6)nc(-c6ccccc6)n5)ccc43)n2)cc1 ; c1ccc(-c2nc(-c3cccc(-c4ccc5c(c4)C(c4ccccc4)(c4ccccc4)c4ccccc4-5)c3)cc(-c3ccc4c(c3)C3c5ccccc5C4c4cc(-c5cc(-c6cccc(-c7ccc8c(c7)C(c7ccccc7)(c7ccccc7)c7ccccc7-8)c6)nc(-c6ccccc6)n5)ccc43)n2)cc1]

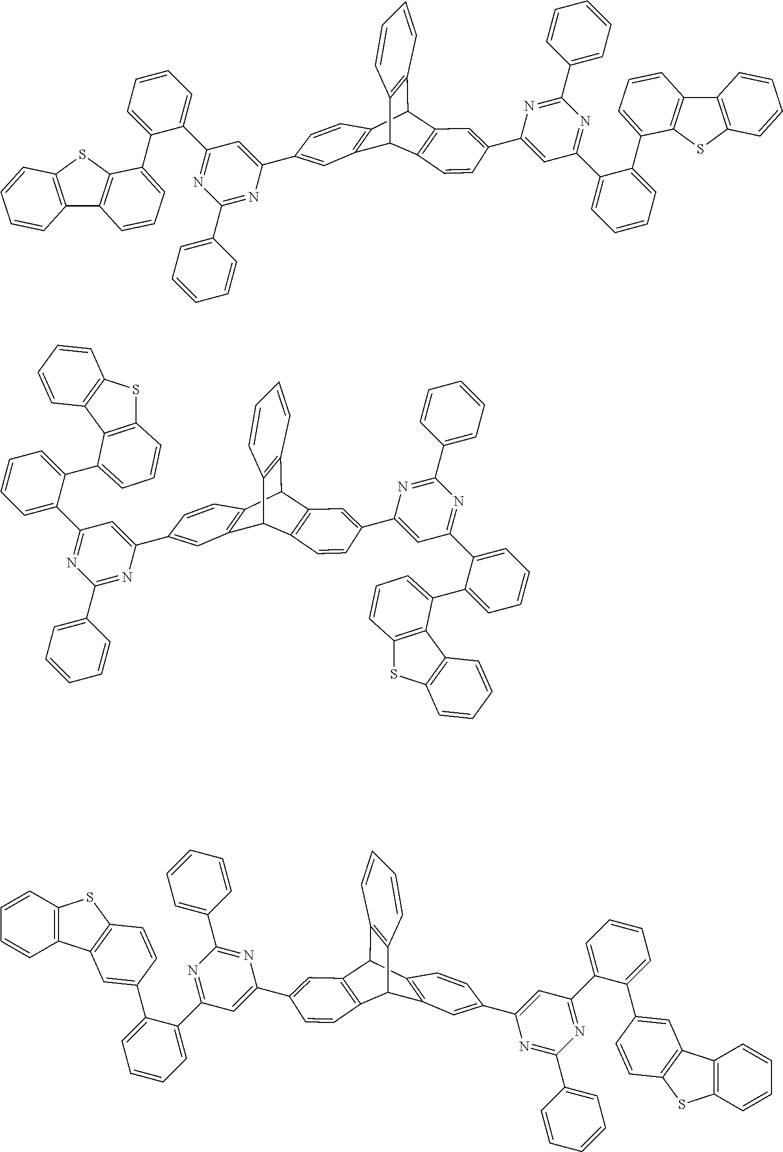 [SMILES: c1ccc(-c2nc(-c3ccc4c(c3)C3c5ccccc5C4c4cc(-c5cc(-c6ccccc6-c6cccc7c6sc6ccccc67)nc(-c6ccccc6)n5)ccc43)cc(-c3ccccc3-c3cccc4c3sc3ccccc34)n2)cc1 ; c1ccc(-c2nc(-c3ccc4c(c3)C3c5ccccc5C4c4cc(-c5cc(-c6ccccc6-c6cccc7sc8ccccc8c67)nc(-c6ccccc6)n5)ccc43)cc(-c3ccccc3-c3cccc4sc5ccccc5c34)n2)cc1 ; c1ccc(-c2nc(-c3ccc4c(c3)C3c5ccccc5C4c4cc(-c5cc(-c6ccccc6-c6ccc7sc8ccccc8c7c6)nc(-c6ccccc6)n5)ccc43)cc(-c3ccccc3-c3ccc4sc5ccccc5c4c3)n2)cc1]

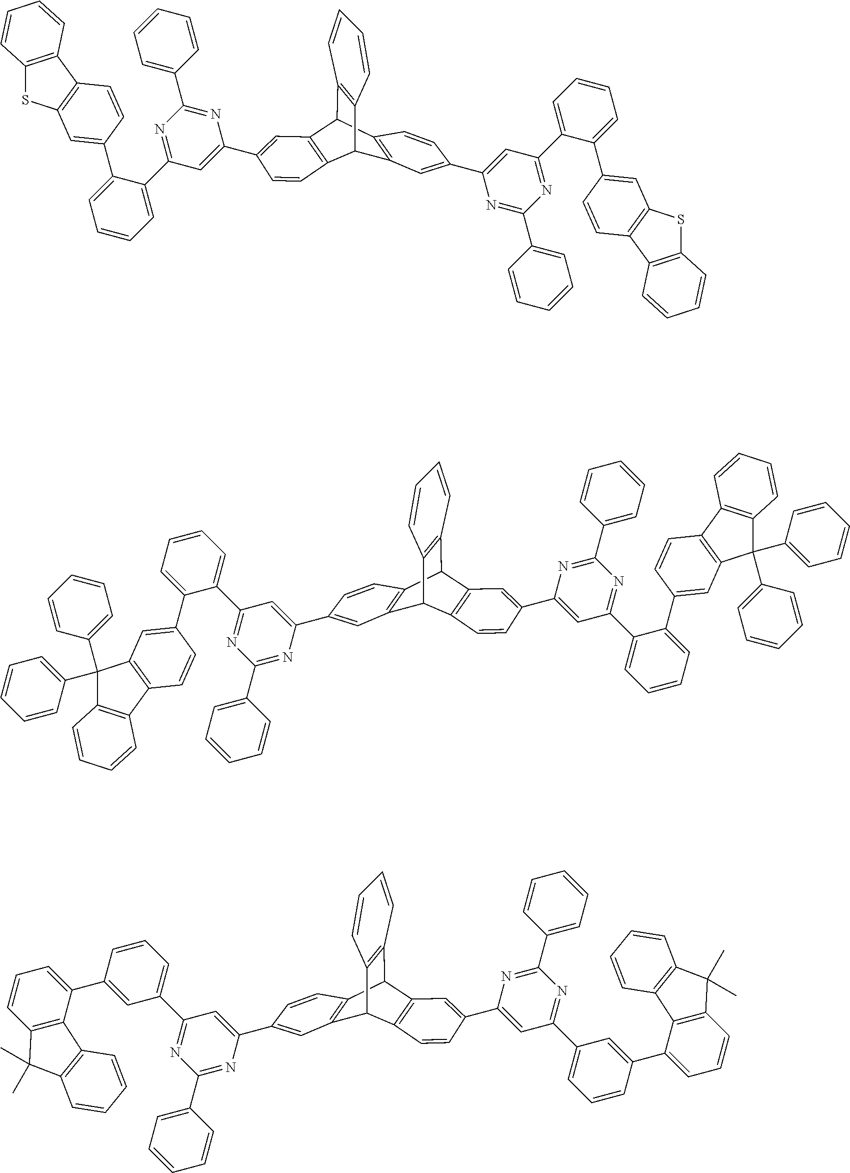

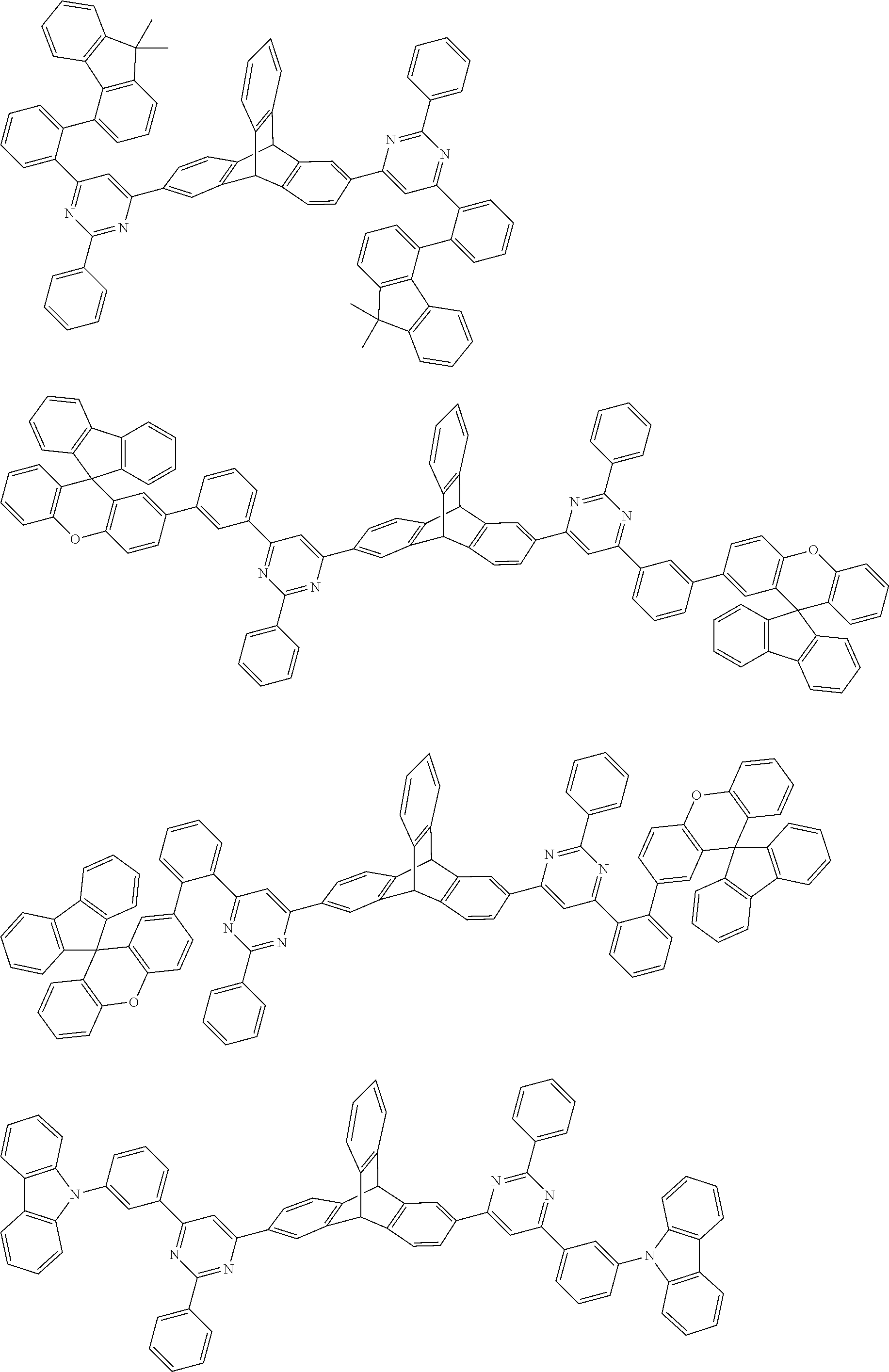

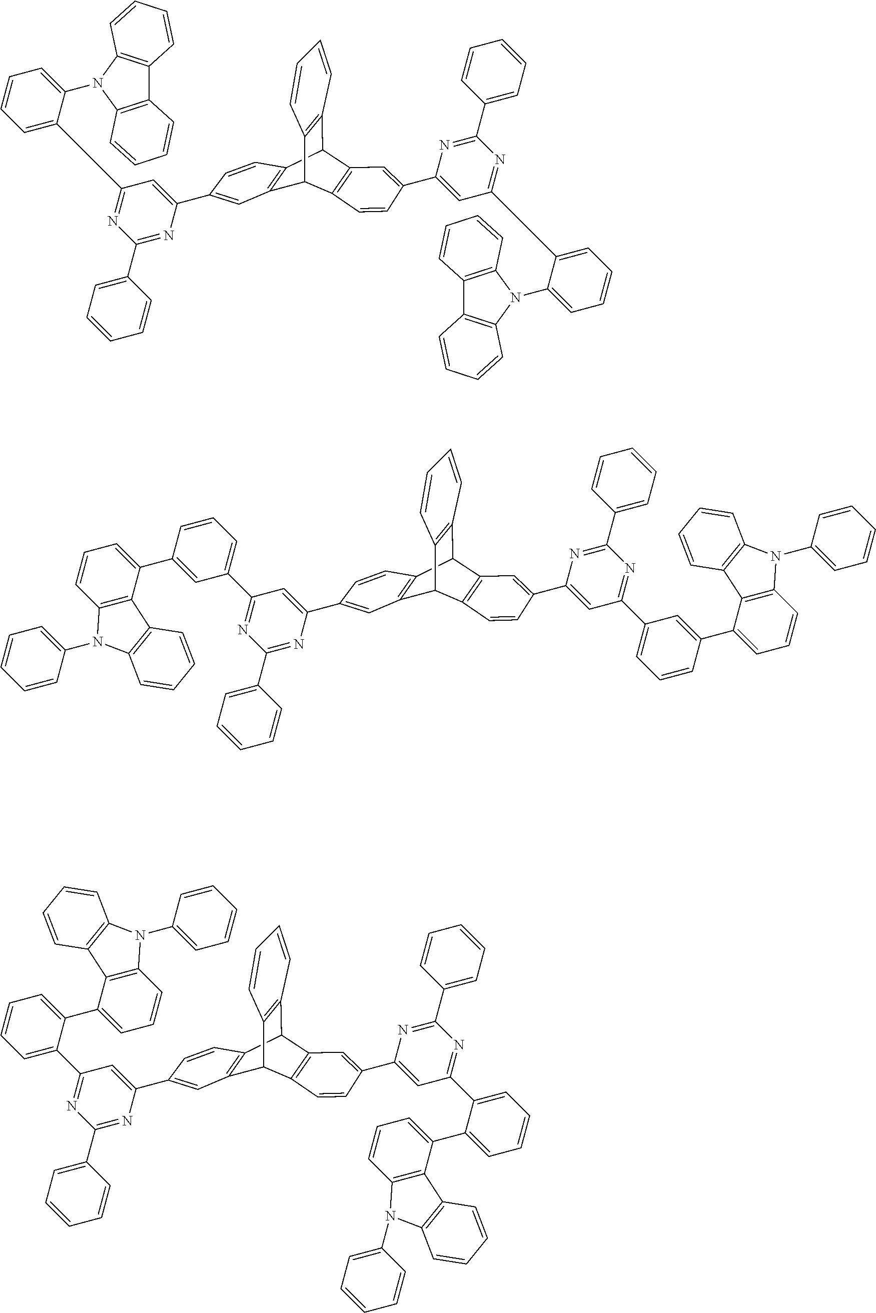 [SMILES: c1ccc(-c2nc(-c3ccc4c(c3)C3c5ccccc5C4c4cc(-c5cc(-c6ccccc6-n6c7ccccc7c7ccccc76)nc(-c6ccccc6)n5)ccc43)cc(-c3ccccc3-n3c4ccccc4c4ccccc43)n2)cc1 ; c1ccc(-c2nc(-c3cccc(-c4cccc5c4c4ccccc4n5-c4ccccc4)c3)cc(-c3ccc4c(c3)C3c5ccccc5C4c4cc(-c5cc(-c6cccc(-c7cccc8c7c7ccccc7n8-c7ccccc7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6cccc7c6c6ccccc6n7-c6ccccc6)nc(-c6ccccc6)n5)ccc43)cc(-c3ccccc3-c3cccc4c3c3ccccc3n4-c3ccccc3)n2)cc1]

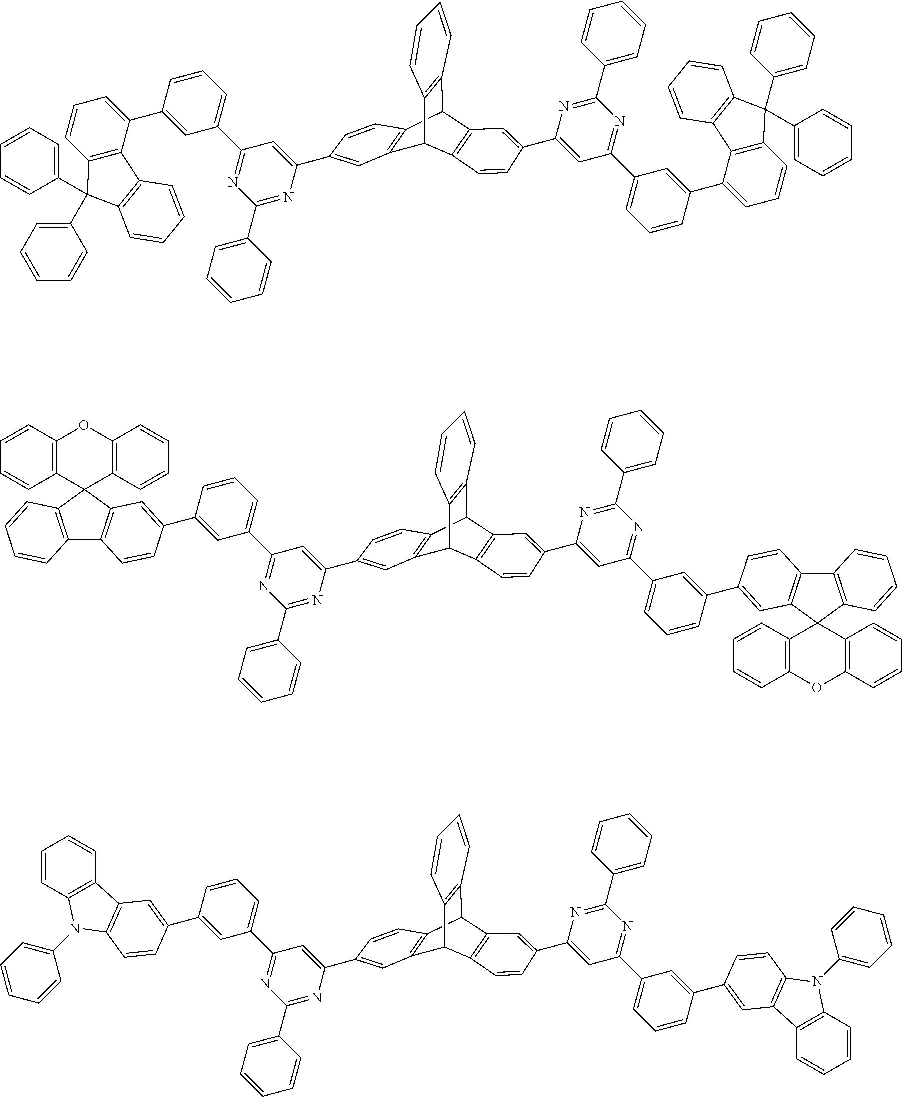

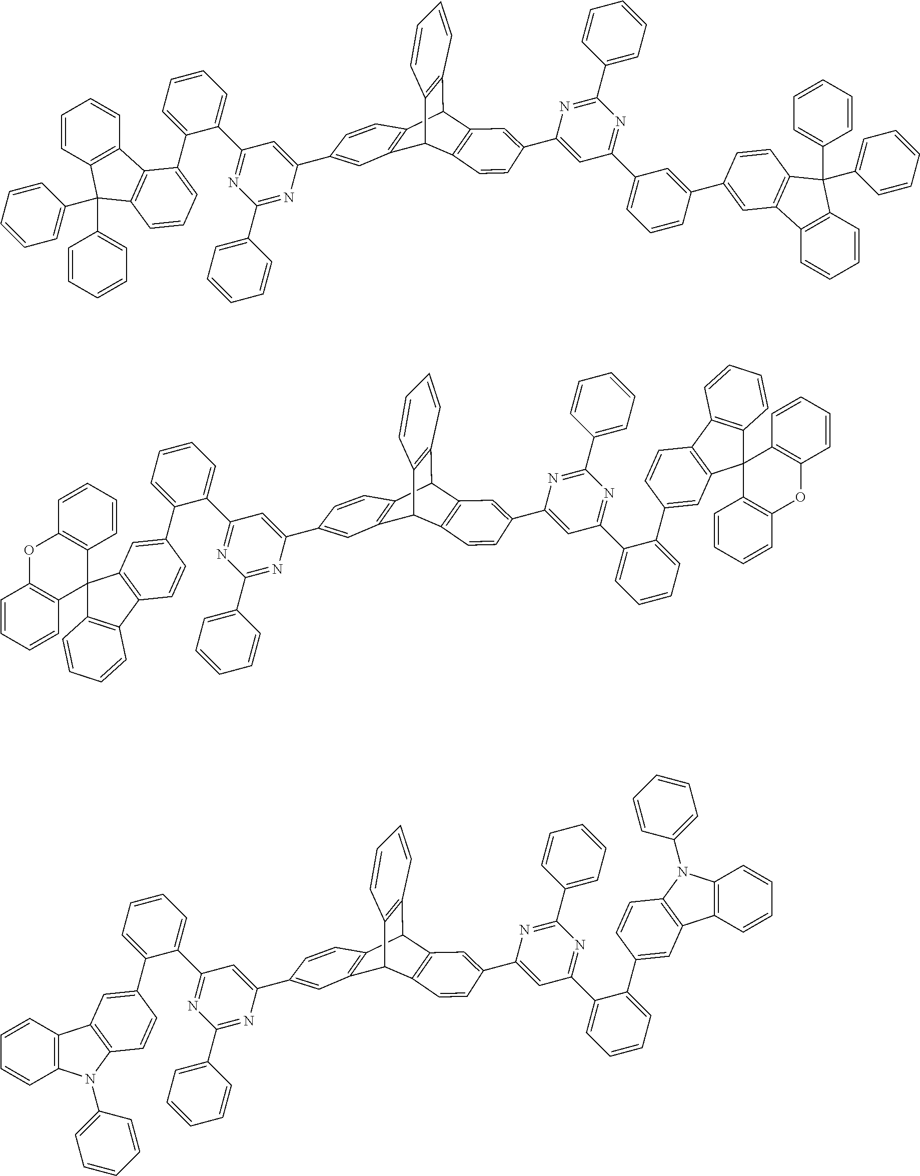

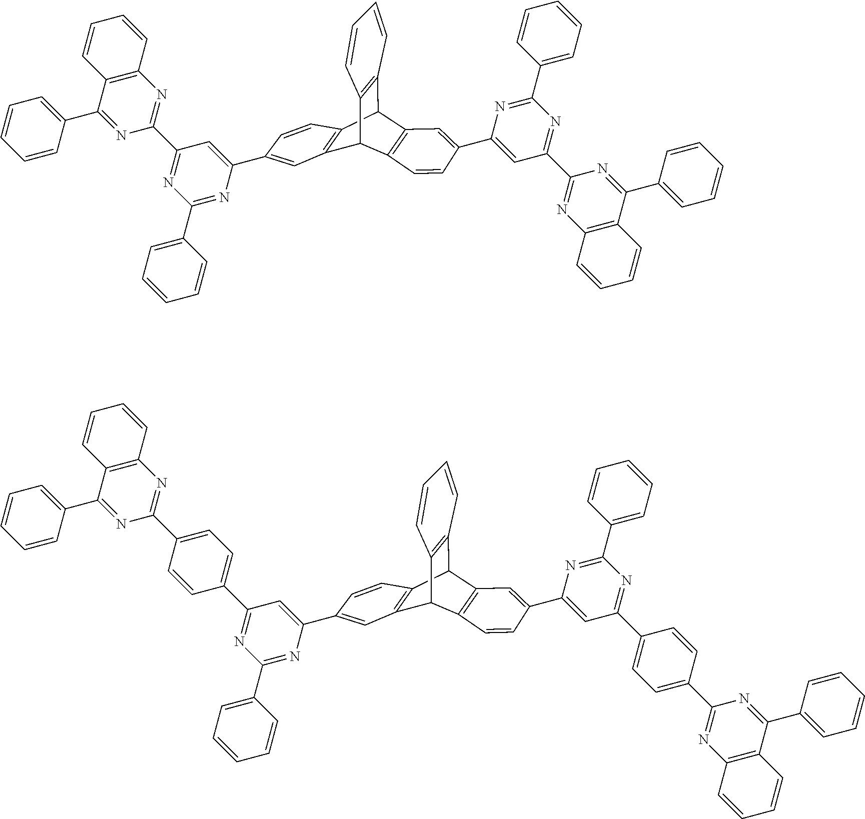 [SMILES: c1ccc(-c2nc(-c3ccc4c(c3)C3c5ccccc5C4c4cc(-c5cc(-c6nc(-c7ccccc7)c7ccccc7n6)nc(-c6ccccc6)n5)ccc43)cc(-c3nc(-c4ccccc4)c4ccccc4n3)n2)cc1 ; c1ccc(-c2nc(-c3ccc(-c4nc(-c5ccccc5)c5ccccc5n4)cc3)cc(-c3ccc4c(c3)C3c5ccccc5C4c4cc(-c5cc(-c6ccc(-c7nc(-c8ccccc8)c8ccccc8n7)cc6)nc(-c6ccccc6)n5)ccc43)n2)cc1]

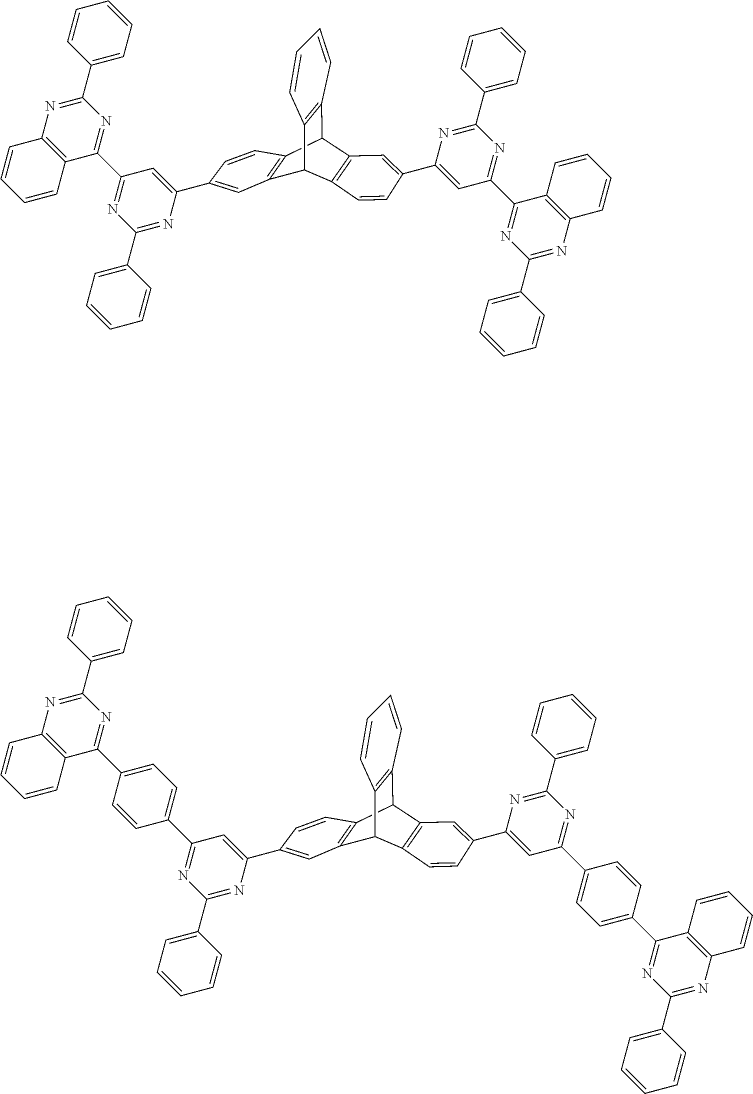 [SMILES: c1ccc(-c2nc(-c3ccc4c(c3)C3c5ccccc5C4c4cc(-c5cc(-c6nc(-c7ccccc7)nc7ccccc67)nc(-c6ccccc6)n5)ccc43)cc(-c3nc(-c4ccccc4)nc4ccccc34)n2)cc1 ; c1ccc(-c2nc(-c3ccc(-c4nc(-c5ccccc5)nc5ccccc45)cc3)cc(-c3ccc4c(c3)C3c5ccccc5C4c4cc(-c5cc(-c6ccc(-c7nc(-c8ccccc8)nc8ccccc78)cc6)nc(-c6ccccc6)n5)ccc43)n2)cc1]

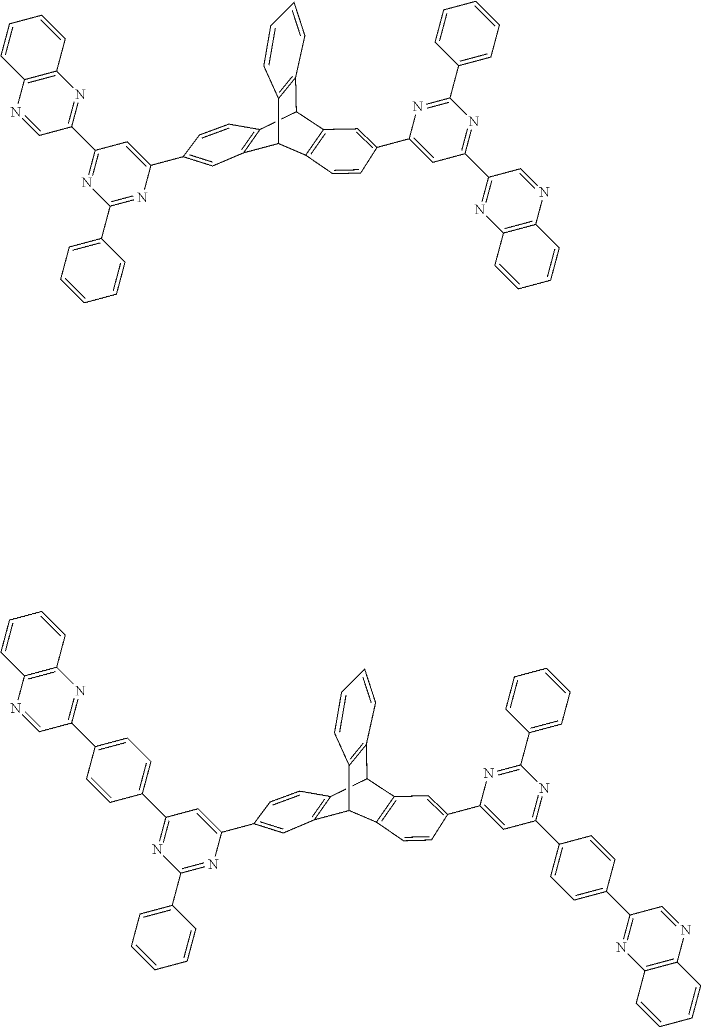 [SMILES: c1ccc(-c2nc(-c3ccc4c(c3)C3c5ccccc5C4c4cc(-c5cc(-c6cnc7ccccc7n6)nc(-c6ccccc6)n5)ccc43)cc(-c3cnc4ccccc4n3)n2)cc1 ; c1ccc(-c2nc(-c3ccc(-c4cnc5ccccc5n4)cc3)cc(-c3ccc4c(c3)C3c5ccccc5C4c4cc(-c5cc(-c6ccc(-c7cnc8ccccc8n7)cc6)nc(-c6ccccc6)n5)ccc43)n2)cc1]

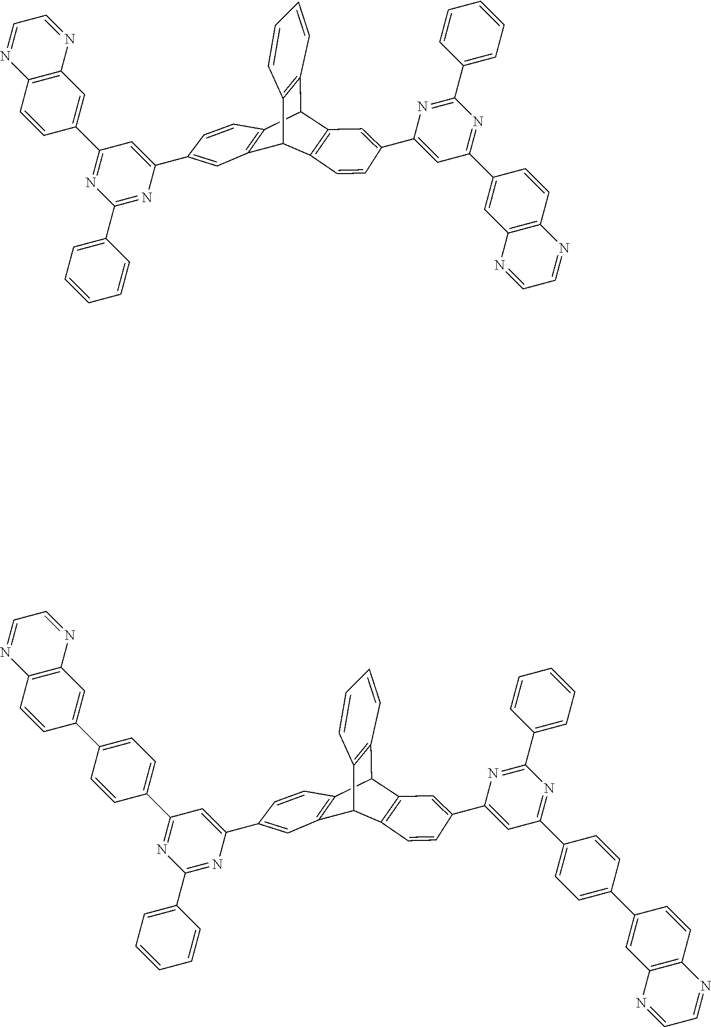 [SMILES: c1ccc(-c2nc(-c3ccc4c(c3)C3c5ccccc5C4c4cc(-c5cc(-c6ccc7nccnc7c6)nc(-c6ccccc6)n5)ccc43)cc(-c3ccc4nccnc4c3)n2)cc1 ; c1ccc(-c2nc(-c3ccc(-c4ccc5nccnc5c4)cc3)cc(-c3ccc4c(c3)C3c5ccccc5C4c4cc(-c5cc(-c6ccc(-c7ccc8nccnc8c7)cc6)nc(-c6ccccc6)n5)ccc43)n2)cc1]

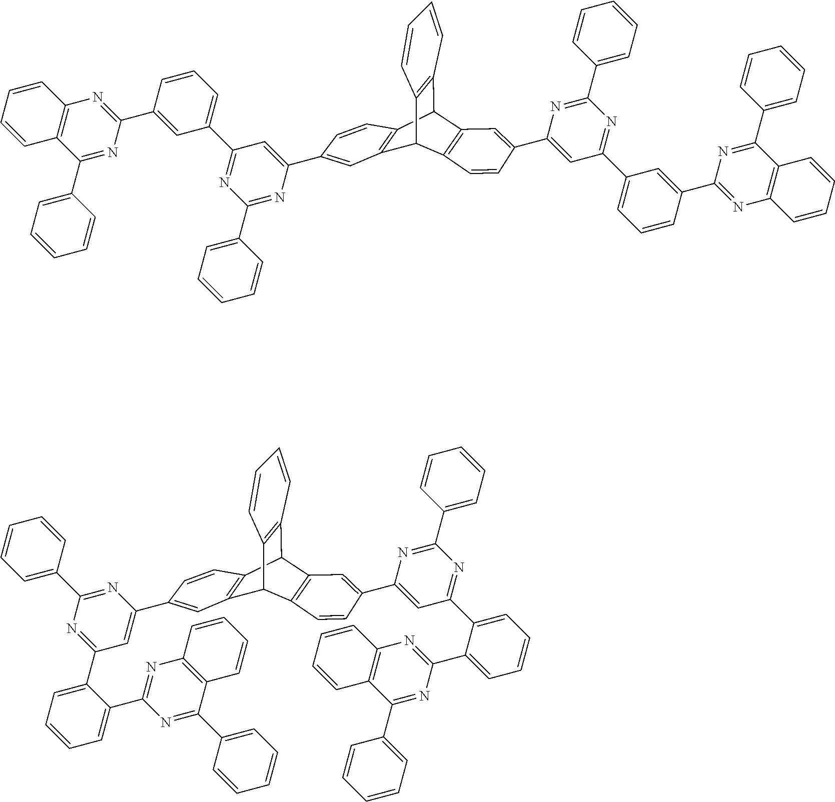 [SMILES: c1ccc(-c2nc(-c3cccc(-c4nc(-c5ccccc5)c5ccccc5n4)c3)cc(-c3ccc4c(c3)C3c5ccccc5C4c4cc(-c5cc(-c6cccc(-c7nc(-c8ccccc8)c8ccccc8n7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6nc(-c7ccccc7)c7ccccc7n6)nc(-c6ccccc6)n5)ccc43)cc(-c3ccccc3-c3nc(-c4ccccc4)c4ccccc4n3)n2)cc1]

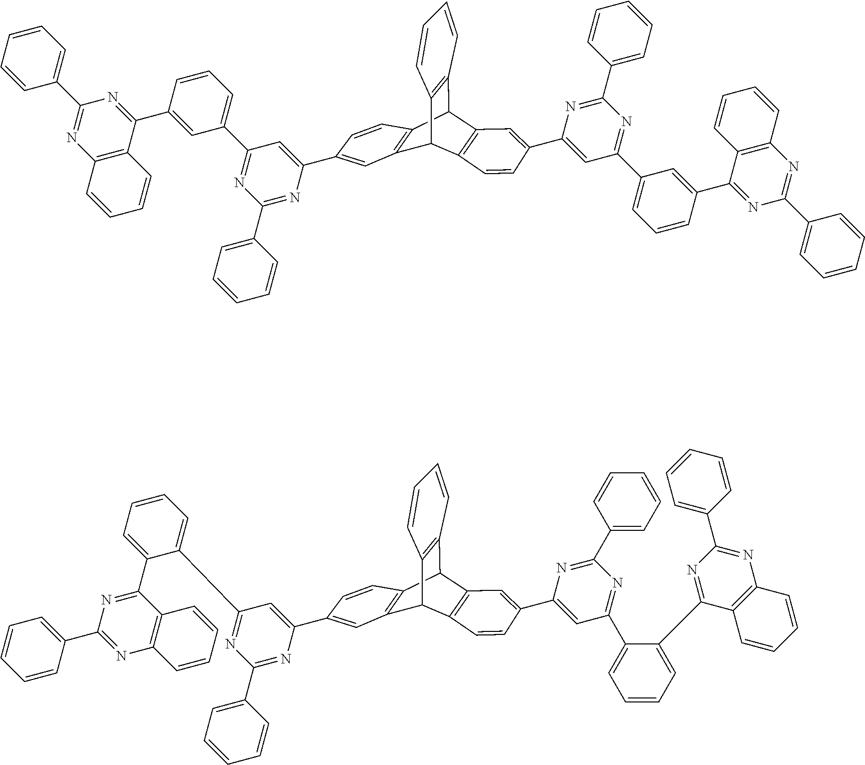 [SMILES: c1ccc(-c2nc(-c3cccc(-c4nc(-c5ccccc5)nc5ccccc45)c3)cc(-c3ccc4c(c3)C3c5ccccc5C4c4cc(-c5cc(-c6cccc(-c7nc(-c8ccccc8)nc8ccccc78)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6nc(-c7ccccc7)nc7ccccc67)nc(-c6ccccc6)n5)ccc43)cc(-c3ccccc3-c3nc(-c4ccccc4)nc4ccccc34)n2)cc1]

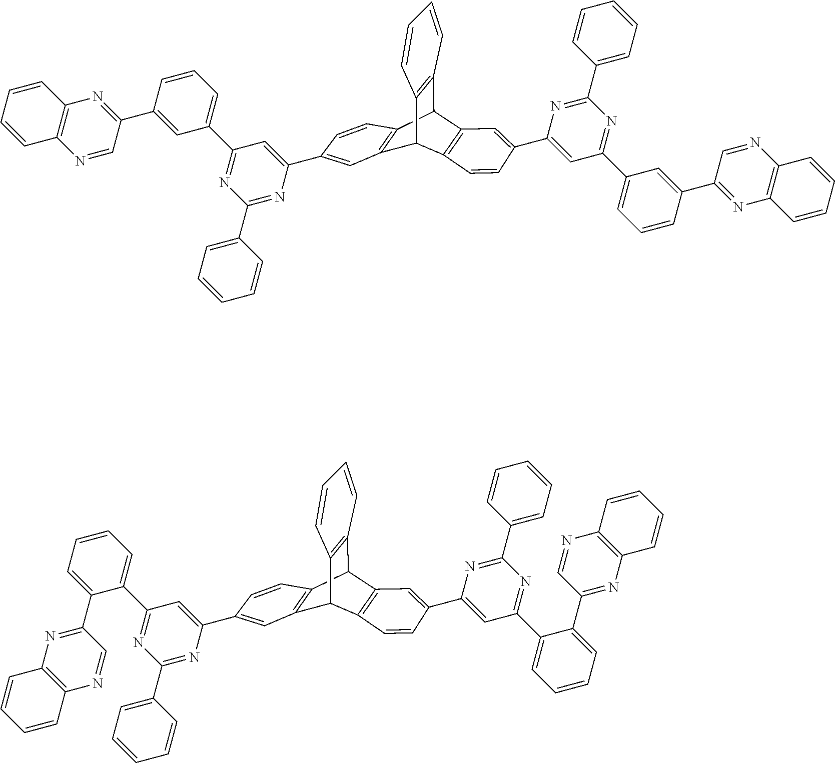 [SMILES: c1ccc(-c2nc(-c3cccc(-c4cnc5ccccc5n4)c3)cc(-c3ccc4c(c3)C3c5ccccc5C4c4cc(-c5cc(-c6cccc(-c7cnc8ccccc8n7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6cnc7ccccc7n6)nc(-c6ccccc6)n5)ccc43)cc(-c3ccccc3-c3cnc4ccccc4n3)n2)cc1]

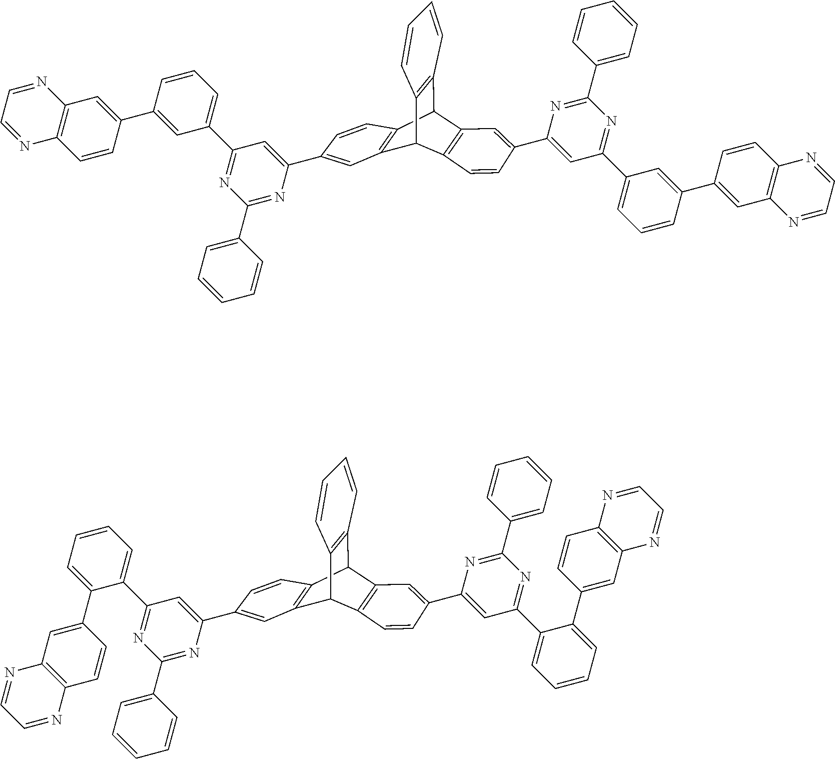 [SMILES: c1ccc(-c2nc(-c3cccc(-c4ccc5nccnc5c4)c3)cc(-c3ccc4c(c3)C3c5ccccc5C4c4cc(-c5cc(-c6cccc(-c7ccc8nccnc8c7)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6ccc7nccnc7c6)nc(-c6ccccc6)n5)ccc43)cc(-c3ccccc3-c3ccc4nccnc4c3)n2)cc1]

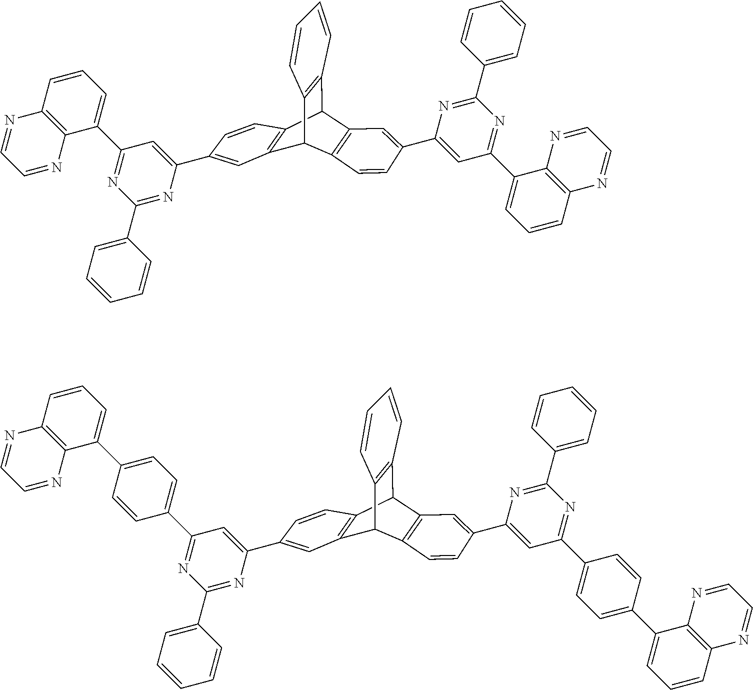 [SMILES: c1ccc(-c2nc(-c3ccc4c(c3)C3c5ccccc5C4c4cc(-c5cc(-c6cccc7nccnc67)nc(-c6ccccc6)n5)ccc43)cc(-c3cccc4nccnc34)n2)cc1 ; c1ccc(-c2nc(-c3ccc(-c4cccc5nccnc45)cc3)cc(-c3ccc4c(c3)C3c5ccccc5C4c4cc(-c5cc(-c6ccc(-c7cccc8nccnc78)cc6)nc(-c6ccccc6)n5)ccc43)n2)cc1]

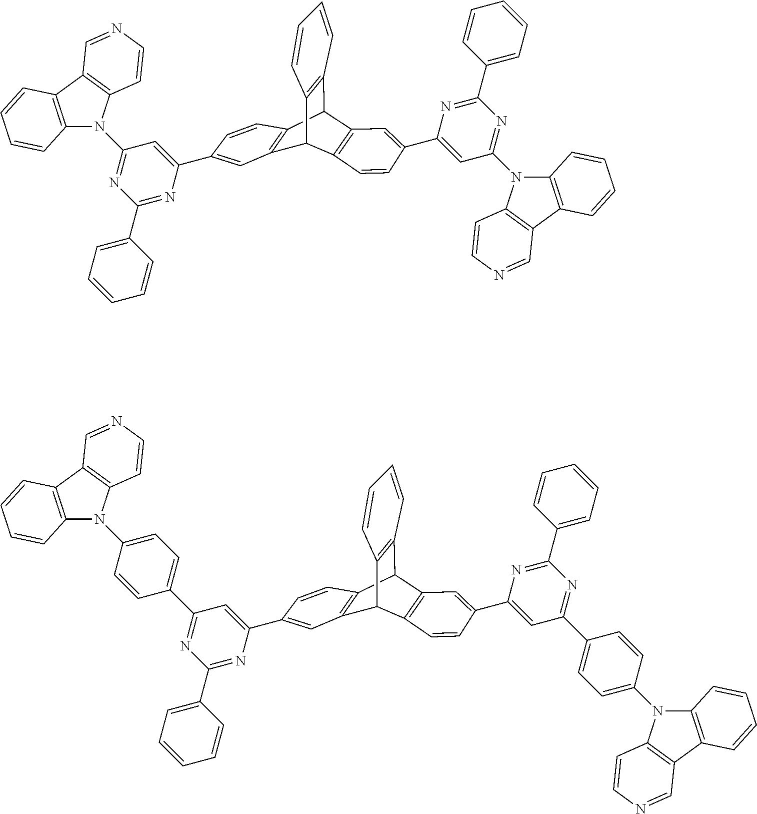 [SMILES: c1ccc(-c2nc(-c3ccc4c(c3)C3c5ccccc5C4c4cc(-c5cc(-n6c7ccccc7c7cnccc76)nc(-c6ccccc6)n5)ccc43)cc(-n3c4ccccc4c4cnccc43)n2)cc1 ; c1ccc(-c2nc(-c3ccc(-n4c5ccccc5c5cnccc54)cc3)cc(-c3ccc4c(c3)C3c5ccccc5C4c4cc(-c5cc(-c6ccc(-n7c8ccccc8c8cnccc87)cc6)nc(-c6ccccc6)n5)ccc43)n2)cc1]

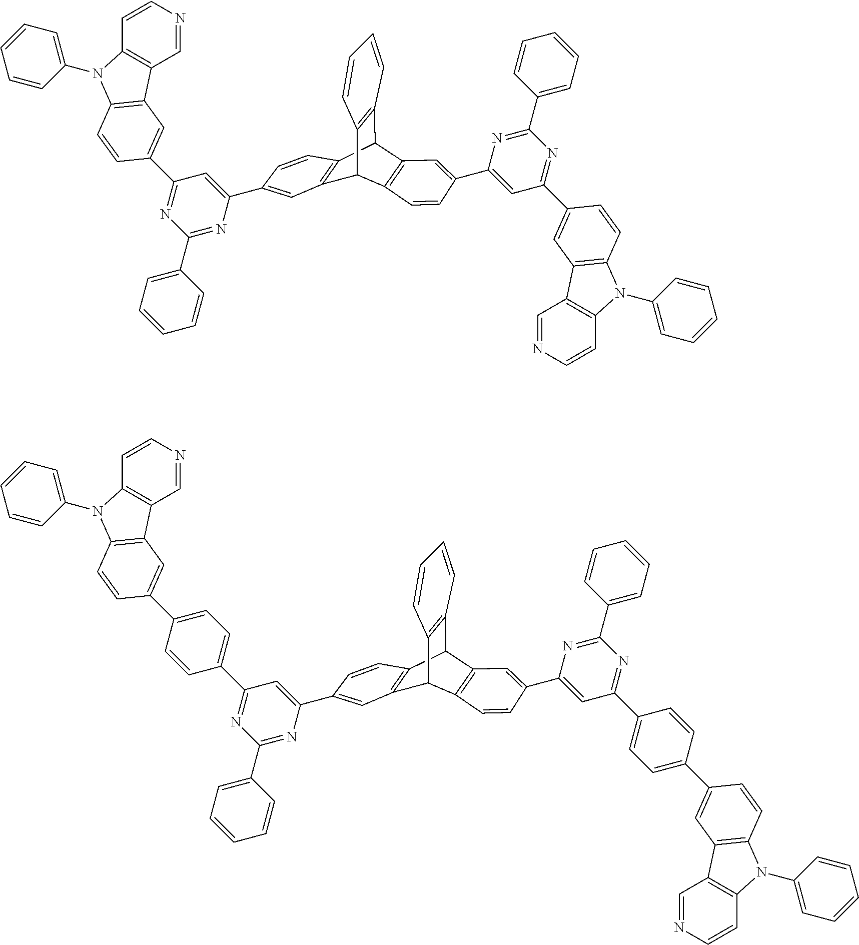 [SMILES: c1ccc(-c2nc(-c3ccc4c(c3)C3c5ccccc5C4c4cc(-c5cc(-c6ccc7c(c6)c6cnccc6n7-c6ccccc6)nc(-c6ccccc6)n5)ccc43)cc(-c3ccc4c(c3)c3cnccc3n4-c3ccccc3)n2)cc1 ; c1ccc(-c2nc(-c3ccc(-c4ccc5c(c4)c4cnccc4n5-c4ccccc4)cc3)cc(-c3ccc4c(c3)C3c5ccccc5C4c4cc(-c5cc(-c6ccc(-c7ccc8c(c7)c7cnccc7n8-c7ccccc7)cc6)nc(-c6ccccc6)n5)ccc43)n2)cc1]

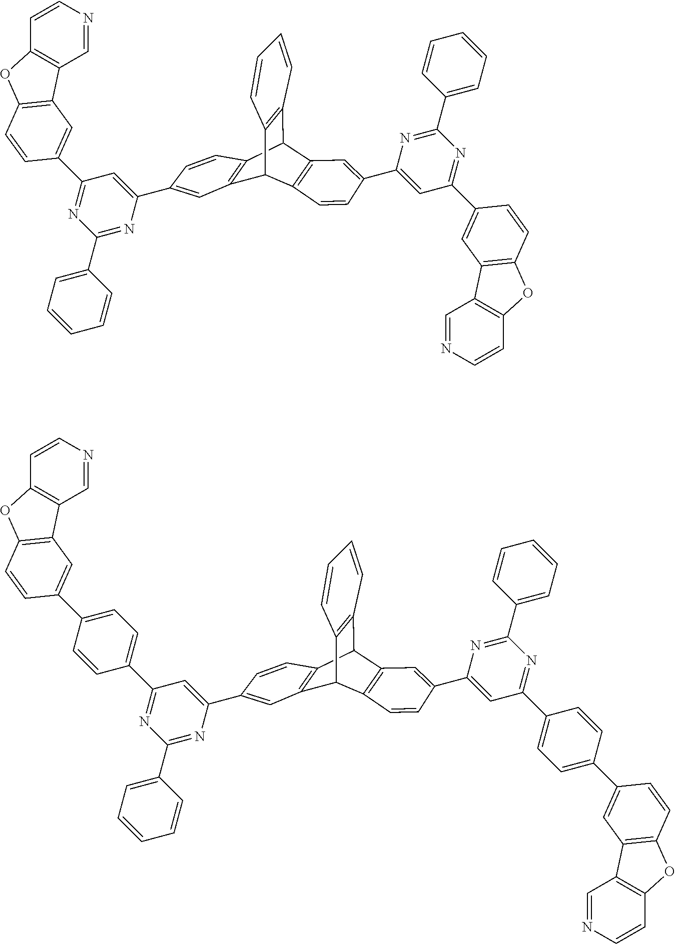 [SMILES: c1ccc(-c2nc(-c3ccc4c(c3)C3c5ccccc5C4c4cc(-c5cc(-c6ccc7oc8ccncc8c7c6)nc(-c6ccccc6)n5)ccc43)cc(-c3ccc4oc5ccncc5c4c3)n2)cc1 ; c1ccc(-c2nc(-c3ccc(-c4ccc5oc6ccncc6c5c4)cc3)cc(-c3ccc4c(c3)C3c5ccccc5C4c4cc(-c5cc(-c6ccc(-c7ccc8oc9ccncc9c8c7)cc6)nc(-c6ccccc6)n5)ccc43)n2)cc1]

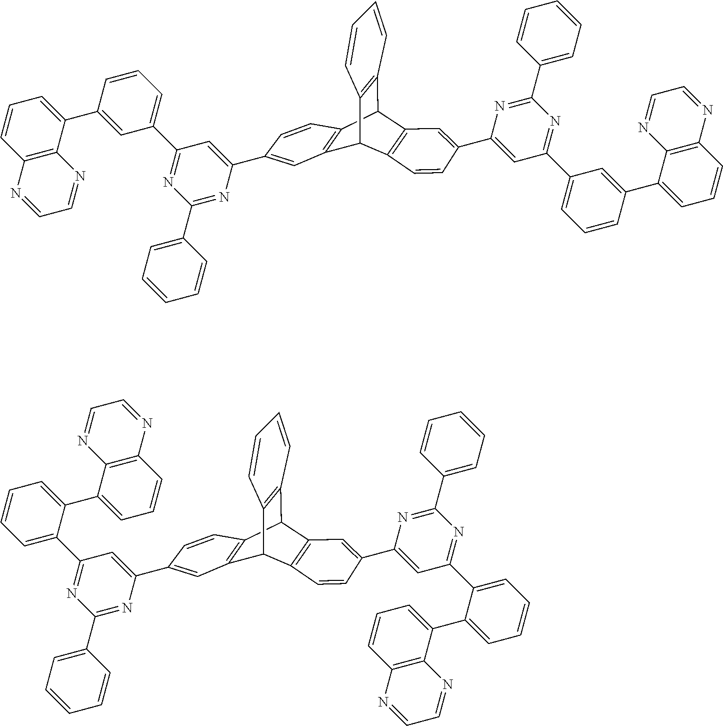 [SMILES: c1ccc(-c2nc(-c3cccc(-c4cccc5nccnc45)c3)cc(-c3ccc4c(c3)C3c5ccccc5C4c4cc(-c5cc(-c6cccc(-c7cccc8nccnc78)c6)nc(-c6ccccc6)n5)ccc43)n2)cc1 ; c1ccc(-c2nc(-c3ccc4c(c3)C3c5ccccc5C4c4cc(-c5cc(-c6ccccc6-c6cccc7nccnc67)nc(-c6ccccc6)n5)ccc43)cc(-c3ccccc3-c3cccc4nccnc34)n2)cc1]

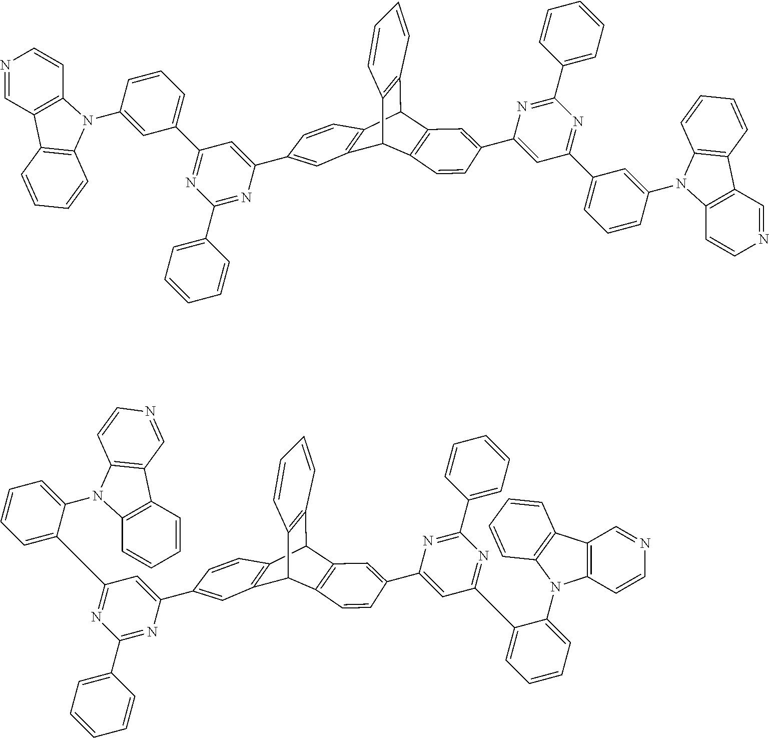 [SMILES: c1ccc(-c2nc(-c3cccc(-n4c5ccccc5c5cnccc54)c3)cc(-c3ccc4c(c3)C3c5ccccc5C4c4cc(-c5cc(-c6cccc(-n7c8ccccc8c8cnccc87)c6)nc(-c6ccccc6)n5)ccc43)n2)cc1 ; c1ccc(-c2nc(-c3ccc4c(c3)C3c5ccccc5C4c4cc(-c5cc(-c6ccccc6-n6c7ccccc7c7cnccc76)nc(-c6ccccc6)n5)ccc43)cc(-c3ccccc3-n3c4ccccc4c4cnccc43)n2)cc1]

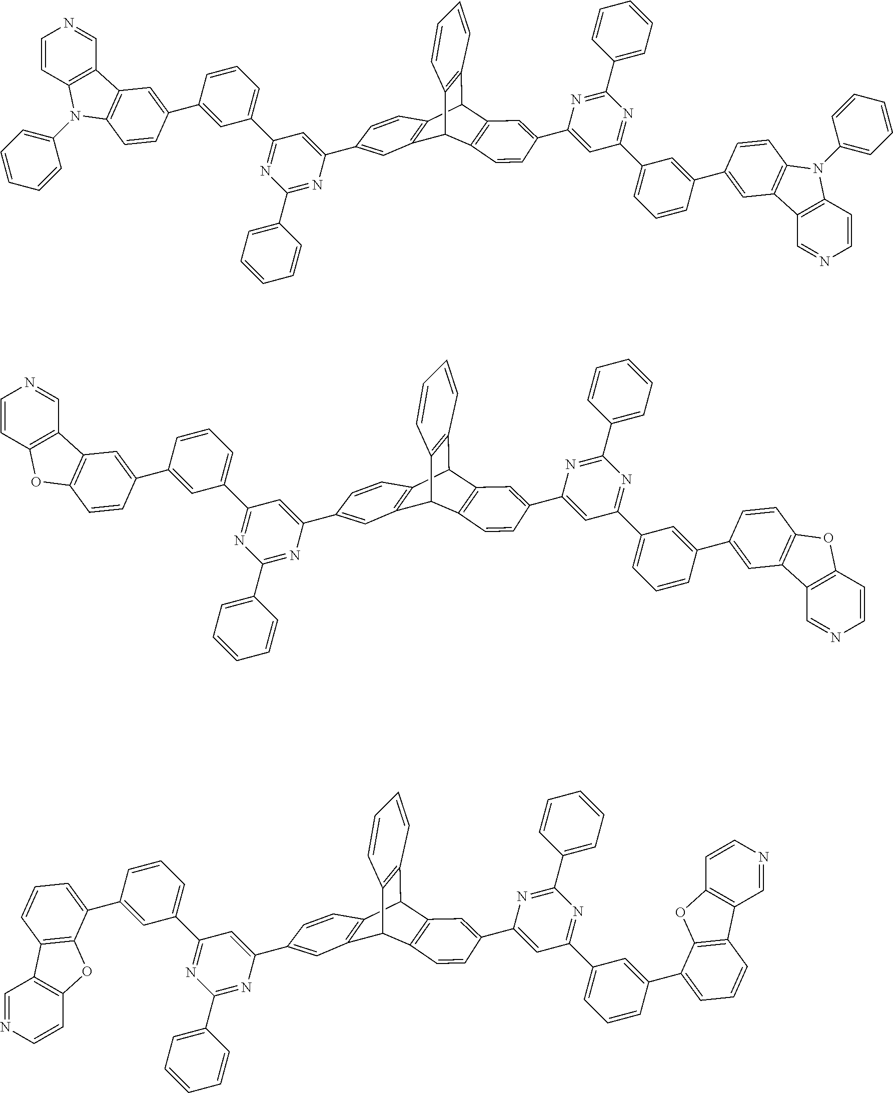 [SMILES: c1ccc(-c2nc(-c3cccc(-c4ccc5c(c4)c4cnccc4n5-c4ccccc4)c3)cc(-c3ccc4c(c3)C3c5ccccc5C4c4cc(-c5cc(-c6cccc(-c7ccc8c(c7)c7cnccc7n8-c7ccccc7)c6)nc(-c6ccccc6)n5)ccc43)n2)cc1 ; c1ccc(-c2nc(-c3cccc(-c4ccc5oc6ccncc6c5c4)c3)cc(-c3ccc4c(c3)C3c5ccccc5C4c4cc(-c5cc(-c6cccc(-c7ccc8oc9ccncc9c8c7)c6)nc(-c6ccccc6)n5)ccc43)n2)cc1 ; c1ccc(-c2nc(-c3cccc(-c4cccc5c4oc4ccncc45)c3)cc(-c3ccc4c(c3)C3c5ccccc5C4c4cc(-c5cc(-c6cccc(-c7cccc8c7oc7ccncc78)c6)nc(-c6ccccc6)n5)ccc43)n2)cc1]

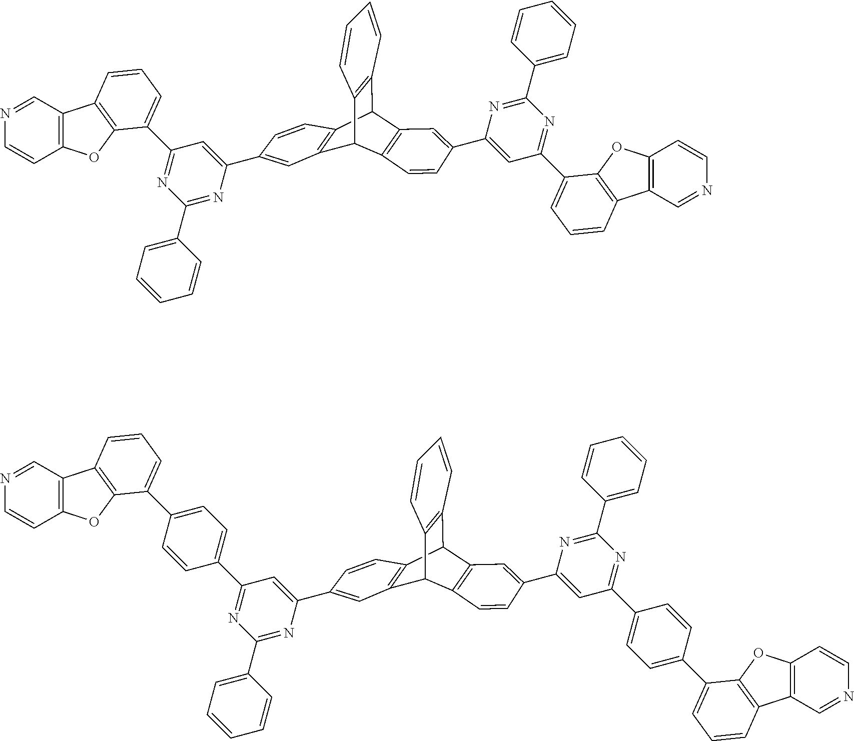 [SMILES: c1ccc(-c2nc(-c3ccc4c(c3)C3c5ccccc5C4c4cc(-c5cc(-c6cccc7c6oc6ccncc67)nc(-c6ccccc6)n5)ccc43)cc(-c3cccc4c3oc3ccncc34)n2)cc1 ; c1ccc(-c2nc(-c3ccc(-c4cccc5c4oc4ccncc45)cc3)cc(-c3ccc4c(c3)C3c5ccccc5C4c4cc(-c5cc(-c6ccc(-c7cccc8c7oc7ccncc78)cc6)nc(-c6ccccc6)n5)ccc43)n2)cc1]

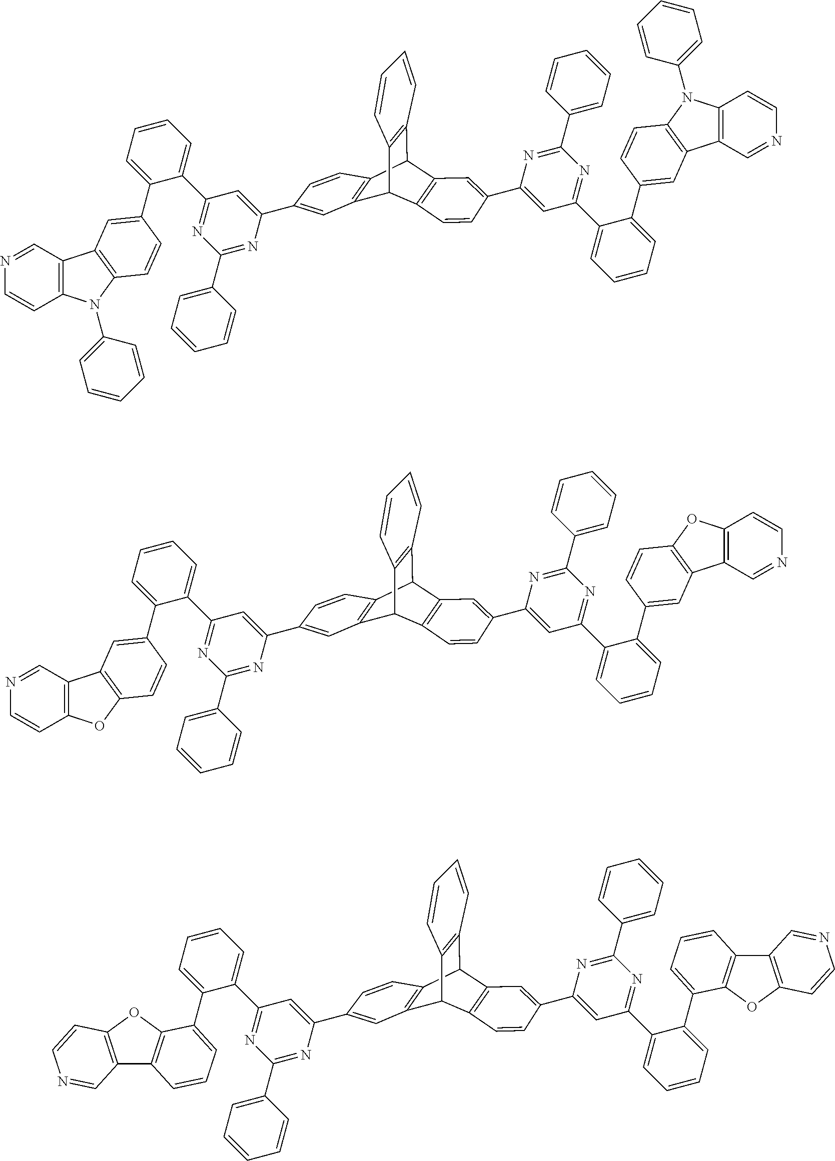 [SMILES: c1ccc(-c2nc(-c3ccc4c(c3)C3c5ccccc5C4c4cc(-c5cc(-c6ccccc6-c6ccc7c(c6)c6cnccc6n7-c6ccccc6)nc(-c6ccccc6)n5)ccc43)cc(-c3ccccc3-c3ccc4c(c3)c3cnccc3n4-c3ccccc3)n2)cc1 ; c1ccc(-c2nc(-c3ccc4c(c3)C3c5ccccc5C4c4cc(-c5cc(-c6ccccc6-c6ccc7oc8ccncc8c7c6)nc(-c6ccccc6)n5)ccc43)cc(-c3ccccc3-c3ccc4oc5ccncc5c4c3)n2)cc1 ; c1ccc(-c2nc(-c3ccc4c(c3)C3c5ccccc5C4c4cc(-c5cc(-c6ccccc6-c6cccc7c6oc6ccncc67)nc(-c6ccccc6)n5)ccc43)cc(-c3ccccc3-c3cccc4c3oc3ccncc34)n2)cc1]

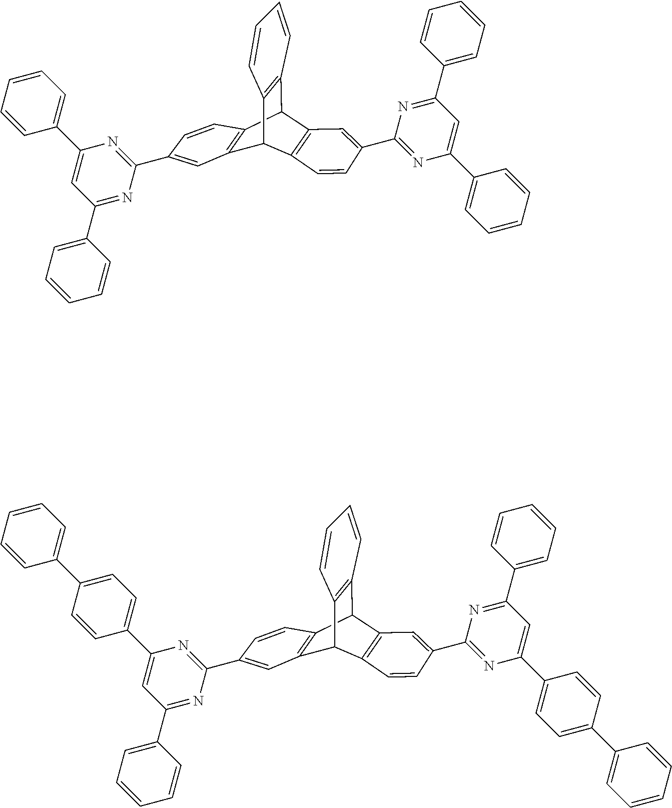 [SMILES: c1ccc(-c2cc(-c3ccccc3)nc(-c3ccc4c(c3)C3c5ccccc5C4c4cc(-c5nc(-c6ccccc6)cc(-c6ccccc6)n5)ccc43)n2)cc1 ; c1ccc(-c2ccc(-c3cc(-c4ccccc4)nc(-c4ccc5c(c4)C4c6ccccc6C5c5cc(-c6nc(-c7ccccc7)cc(-c7ccc(-c8ccccc8)cc7)n6)ccc54)n3)cc2)cc1]

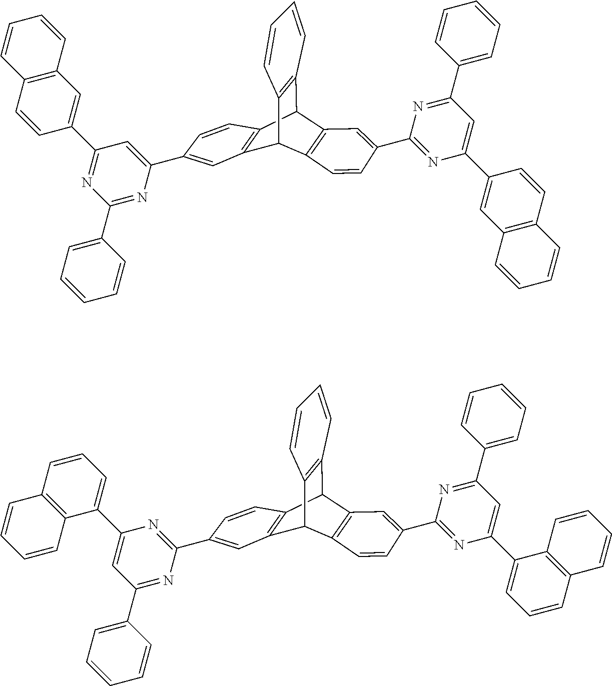 [SMILES: c1ccc(-c2cc(-c3ccc4ccccc4c3)nc(-c3ccc4c(c3)C3c5ccccc5C4c4cc(-c5cc(-c6ccc7ccccc7c6)nc(-c6ccccc6)n5)ccc43)n2)cc1 ; c1ccc(-c2cc(-c3cccc4ccccc34)nc(-c3ccc4c(c3)C3c5ccccc5C4c4cc(-c5nc(-c6ccccc6)cc(-c6cccc7ccccc67)n5)ccc43)n2)cc1]

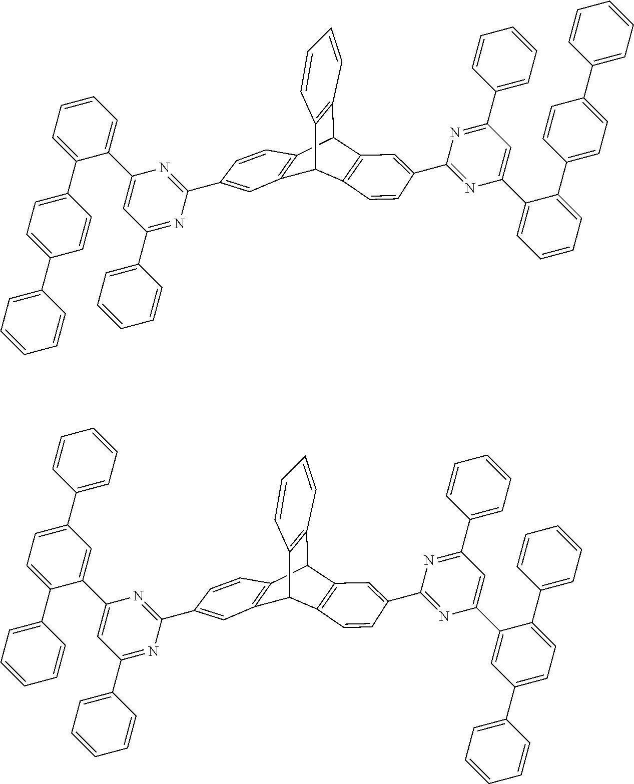 [SMILES: c1ccc(-c2ccc(-c3ccccc3-c3cc(-c4ccccc4)nc(-c4ccc5c(c4)C4c6ccccc6C5c5cc(-c6nc(-c7ccccc7)cc(-c7ccccc7-c7ccc(-c8ccccc8)cc7)n6)ccc54)n3)cc2)cc1 ; c1ccc(-c2ccc(-c3ccccc3)c(-c3cc(-c4ccccc4)nc(-c4ccc5c(c4)C4c6ccccc6C5c5cc(-c6nc(-c7ccccc7)cc(-c7cc(-c8ccccc8)ccc7-c7ccccc7)n6)ccc54)n3)c2)cc1]

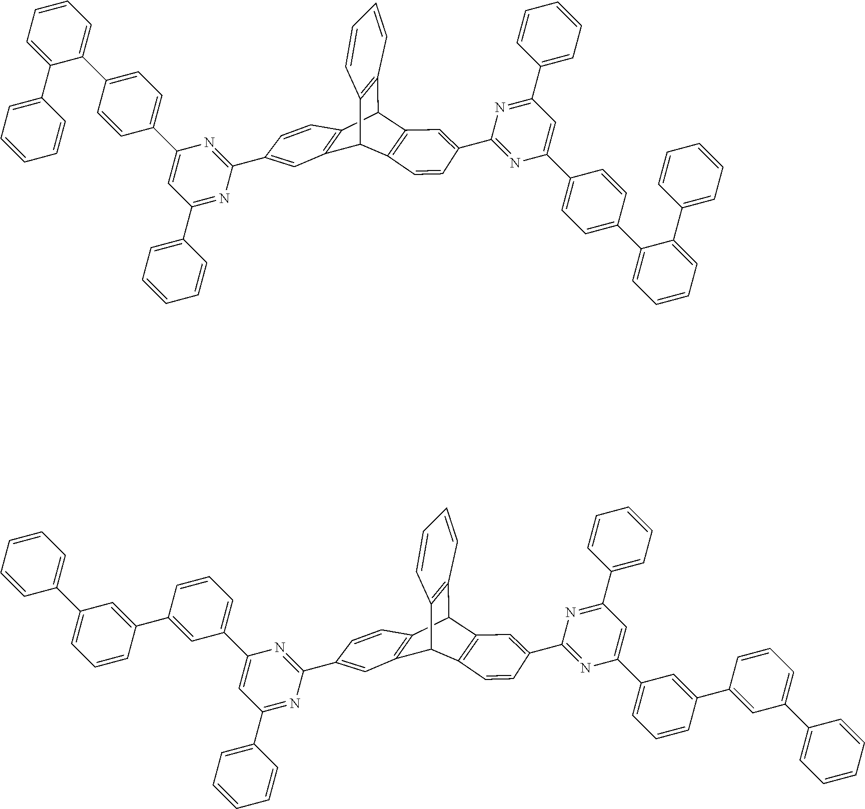 [SMILES: c1ccc(-c2cc(-c3ccc(-c4ccccc4-c4ccccc4)cc3)nc(-c3ccc4c(c3)C3c5ccccc5C4c4cc(-c5nc(-c6ccccc6)cc(-c6ccc(-c7ccccc7-c7ccccc7)cc6)n5)ccc43)n2)cc1 ; c1ccc(-c2cccc(-c3cccc(-c4cc(-c5ccccc5)nc(-c5ccc6c(c5)C5c7ccccc7C6c6cc(-c7nc(-c8ccccc8)cc(-c8cccc(-c9cccc(-c%10ccccc%10)c9)c8)n7)ccc65)n4)c3)c2)cc1]

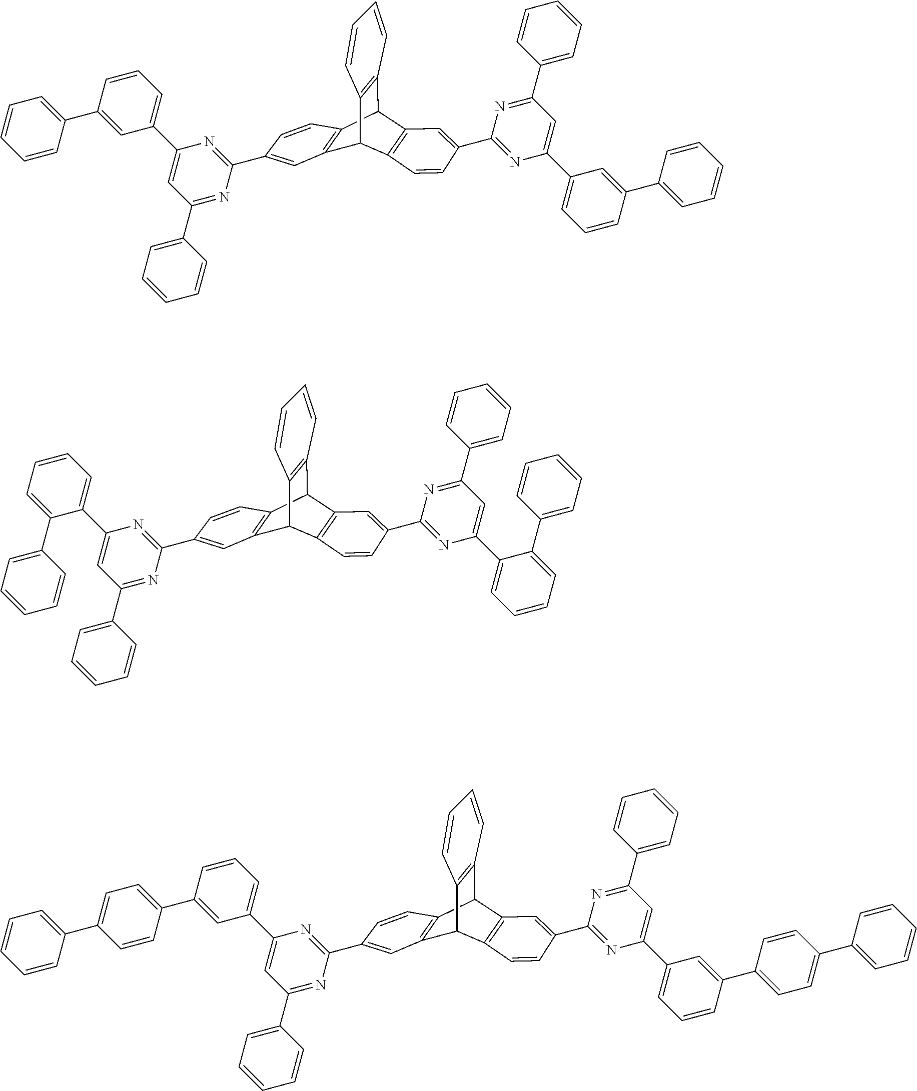 [SMILES: c1ccc(-c2cccc(-c3cc(-c4ccccc4)nc(-c4ccc5c(c4)C4c6ccccc6C5c5cc(-c6nc(-c7ccccc7)cc(-c7cccc(-c8ccccc8)c7)n6)ccc54)n3)c2)cc1 ; c1ccc(-c2cc(-c3ccccc3-c3ccccc3)nc(-c3ccc4c(c3)C3c5ccccc5C4c4cc(-c5nc(-c6ccccc6)cc(-c6ccccc6-c6ccccc6)n5)ccc43)n2)cc1 ; c1ccc(-c2ccc(-c3cccc(-c4cc(-c5ccccc5)nc(-c5ccc6c(c5)C5c7ccccc7C6c6cc(-c7nc(-c8ccccc8)cc(-c8cccc(-c9ccc(-c%10ccccc%10)cc9)c8)n7)ccc65)n4)c3)cc2)cc1]

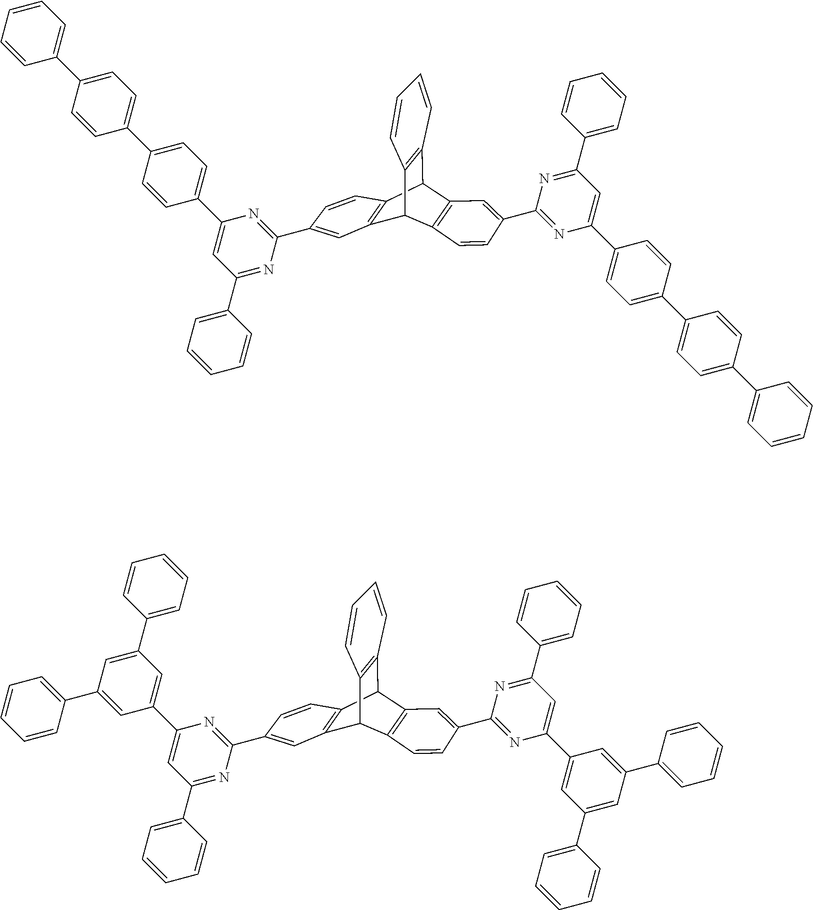 [SMILES: c1ccc(-c2ccc(-c3ccc(-c4cc(-c5ccccc5)nc(-c5ccc6c(c5)C5c7ccccc7C6c6cc(-c7nc(-c8ccccc8)cc(-c8ccc(-c9ccc(-c%10ccccc%10)cc9)cc8)n7)ccc65)n4)cc3)cc2)cc1 ; c1ccc(-c2cc(-c3ccccc3)cc(-c3cc(-c4ccccc4)nc(-c4ccc5c(c4)C4c6ccccc6C5c5cc(-c6nc(-c7ccccc7)cc(-c7cc(-c8ccccc8)cc(-c8ccccc8)c7)n6)ccc54)n3)c2)cc1]

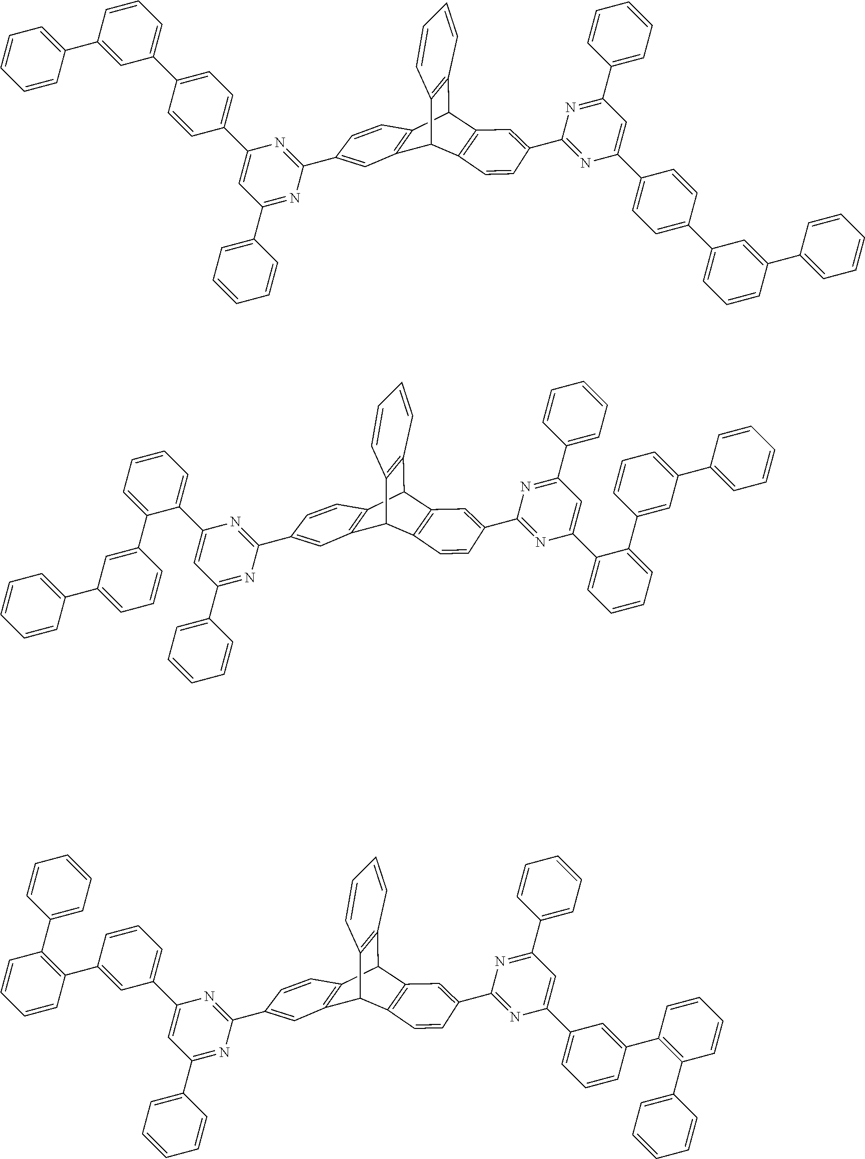 [SMILES: c1ccc(-c2cccc(-c3ccc(-c4cc(-c5ccccc5)nc(-c5ccc6c(c5)C5c7ccccc7C6c6cc(-c7nc(-c8ccccc8)cc(-c8ccc(-c9cccc(-c%10ccccc%10)c9)cc8)n7)ccc65)n4)cc3)c2)cc1 ; c1ccc(-c2cccc(-c3ccccc3-c3cc(-c4ccccc4)nc(-c4ccc5c(c4)C4c6ccccc6C5c5cc(-c6nc(-c7ccccc7)cc(-c7ccccc7-c7cccc(-c8ccccc8)c7)n6)ccc54)n3)c2)cc1 ; c1ccc(-c2cc(-c3cccc(-c4ccccc4-c4ccccc4)c3)nc(-c3ccc4c(c3)C3c5ccccc5C4c4cc(-c5nc(-c6ccccc6)cc(-c6cccc(-c7ccccc7-c7ccccc7)c6)n5)ccc43)n2)cc1]

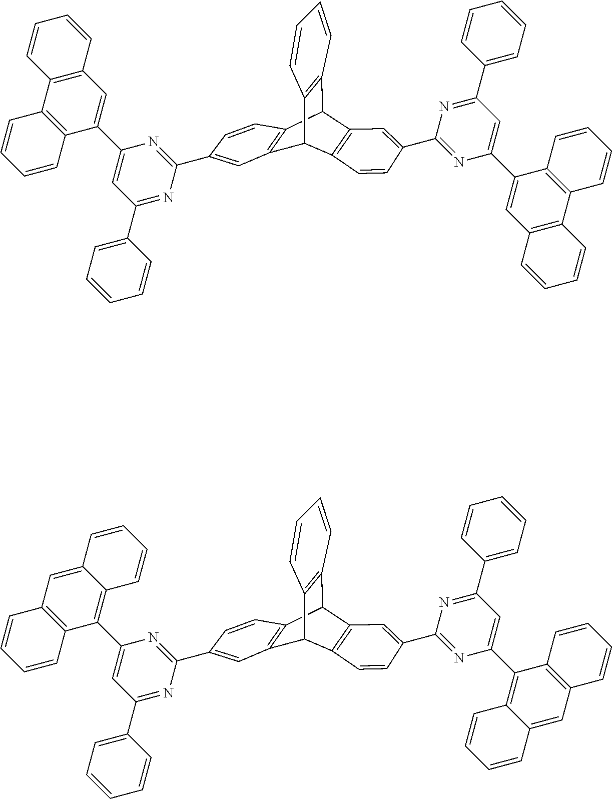 [SMILES: c1ccc(-c2cc(-c3cc4ccccc4c4ccccc34)nc(-c3ccc4c(c3)C3c5ccccc5C4c4cc(-c5nc(-c6ccccc6)cc(-c6cc7ccccc7c7ccccc67)n5)ccc43)n2)cc1 ; c1ccc(-c2cc(-c3c4ccccc4cc4ccccc34)nc(-c3ccc4c(c3)C3c5ccccc5C4c4cc(-c5nc(-c6ccccc6)cc(-c6c7ccccc7cc7ccccc67)n5)ccc43)n2)cc1]

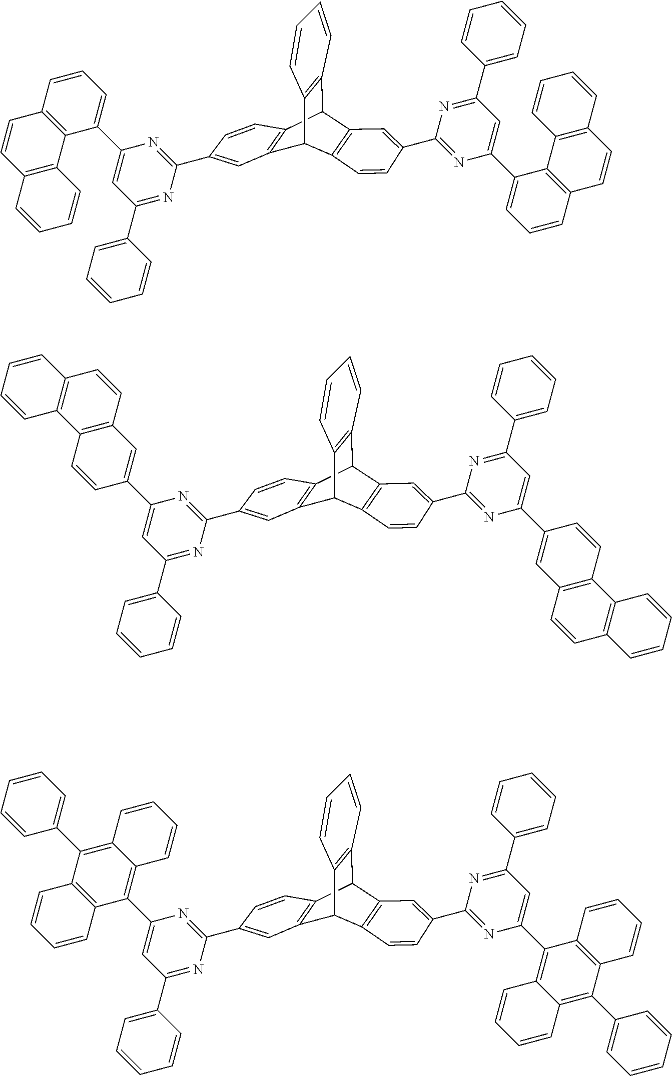 [SMILES: c1ccc(-c2cc(-c3cccc4ccc5ccccc5c34)nc(-c3ccc4c(c3)C3c5ccccc5C4c4cc(-c5nc(-c6ccccc6)cc(-c6cccc7ccc8ccccc8c67)n5)ccc43)n2)cc1 ; c1ccc(-c2cc(-c3ccc4c(ccc5ccccc54)c3)nc(-c3ccc4c(c3)C3c5ccccc5C4c4cc(-c5nc(-c6ccccc6)cc(-c6ccc7c(ccc8ccccc87)c6)n5)ccc43)n2)cc1 ; c1ccc(-c2cc(-c3c4ccccc4c(-c4ccccc4)c4ccccc34)nc(-c3ccc4c(c3)C3c5ccccc5C4c4cc(-c5nc(-c6ccccc6)cc(-c6c7ccccc7c(-c7ccccc7)c7ccccc67)n5)ccc43)n2)cc1]

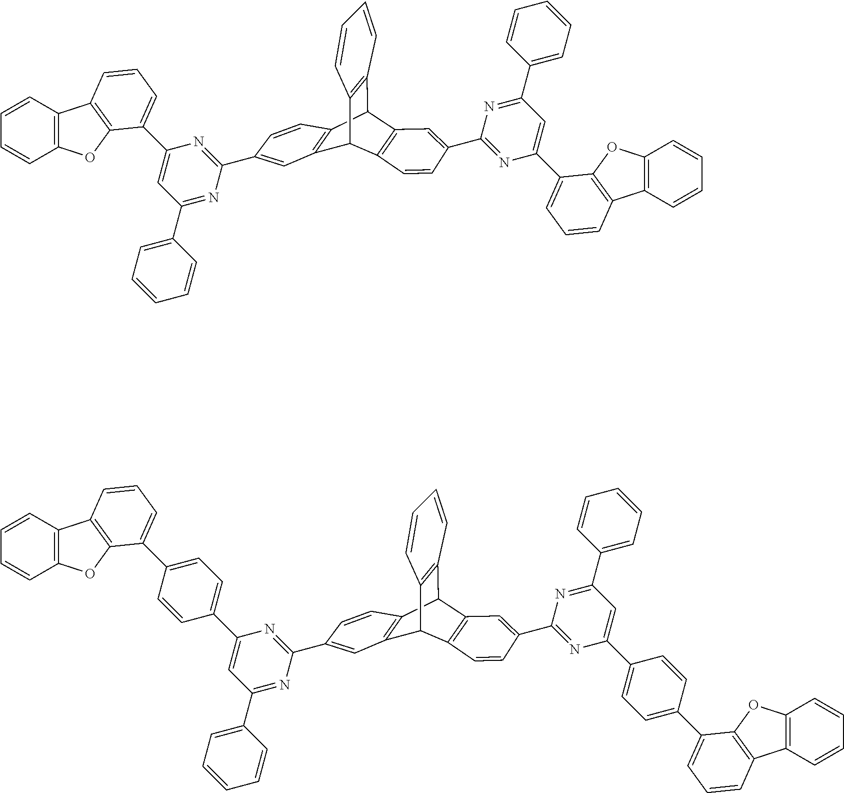 [SMILES: c1ccc(-c2cc(-c3cccc4c3oc3ccccc34)nc(-c3ccc4c(c3)C3c5ccccc5C4c4cc(-c5nc(-c6ccccc6)cc(-c6cccc7c6oc6ccccc67)n5)ccc43)n2)cc1 ; c1ccc(-c2cc(-c3ccc(-c4cccc5c4oc4ccccc45)cc3)nc(-c3ccc4c(c3)C3c5ccccc5C4c4cc(-c5nc(-c6ccccc6)cc(-c6ccc(-c7cccc8c7oc7ccccc78)cc6)n5)ccc43)n2)cc1]

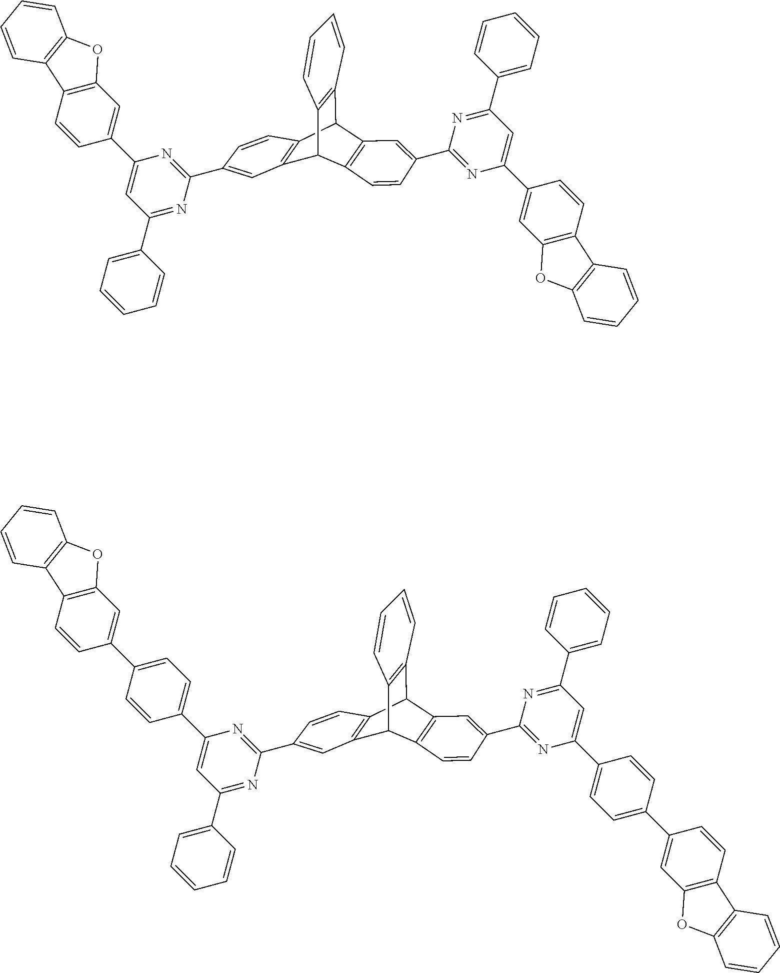 [SMILES: c1ccc(-c2cc(-c3ccc4c(c3)oc3ccccc34)nc(-c3ccc4c(c3)C3c5ccccc5C4c4cc(-c5nc(-c6ccccc6)cc(-c6ccc7c(c6)oc6ccccc67)n5)ccc43)n2)cc1 ; c1ccc(-c2cc(-c3ccc(-c4ccc5c(c4)oc4ccccc45)cc3)nc(-c3ccc4c(c3)C3c5ccccc5C4c4cc(-c5nc(-c6ccccc6)cc(-c6ccc(-c7ccc8c(c7)oc7ccccc78)cc6)n5)ccc43)n2)cc1]

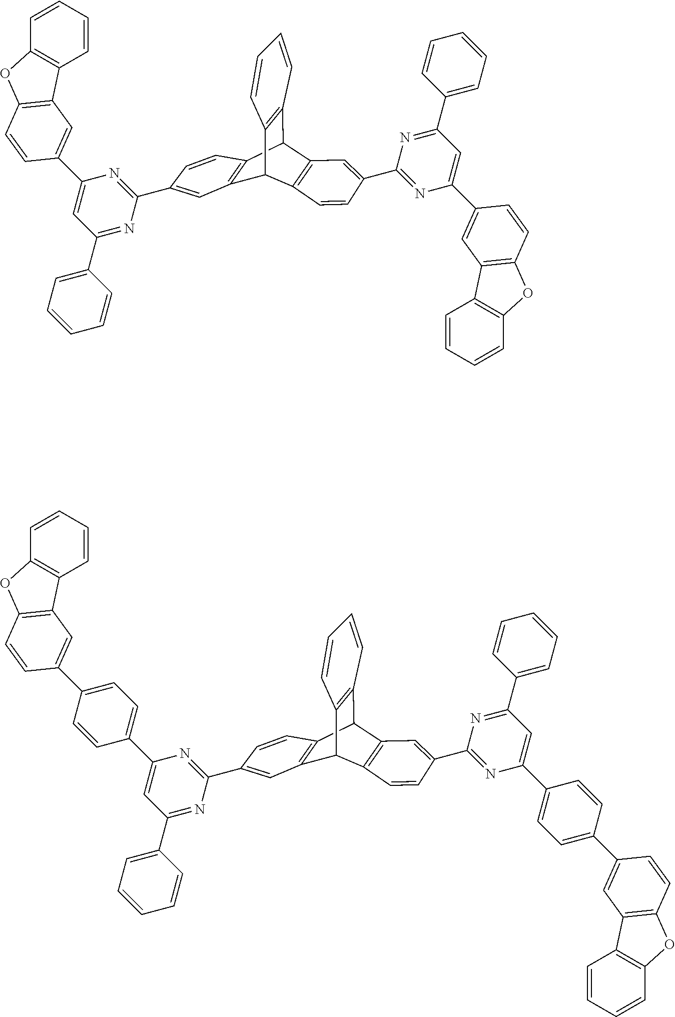 [SMILES: c1ccc(-c2cc(-c3ccc4oc5ccccc5c4c3)nc(-c3ccc4c(c3)C3c5ccccc5C4c4cc(-c5nc(-c6ccccc6)cc(-c6ccc7oc8ccccc8c7c6)n5)ccc43)n2)cc1 ; c1ccc(-c2cc(-c3ccc(-c4ccc5oc6ccccc6c5c4)cc3)nc(-c3ccc4c(c3)C3c5ccccc5C4c4cc(-c5nc(-c6ccccc6)cc(-c6ccc(-c7ccc8oc9ccccc9c8c7)cc6)n5)ccc43)n2)cc1]

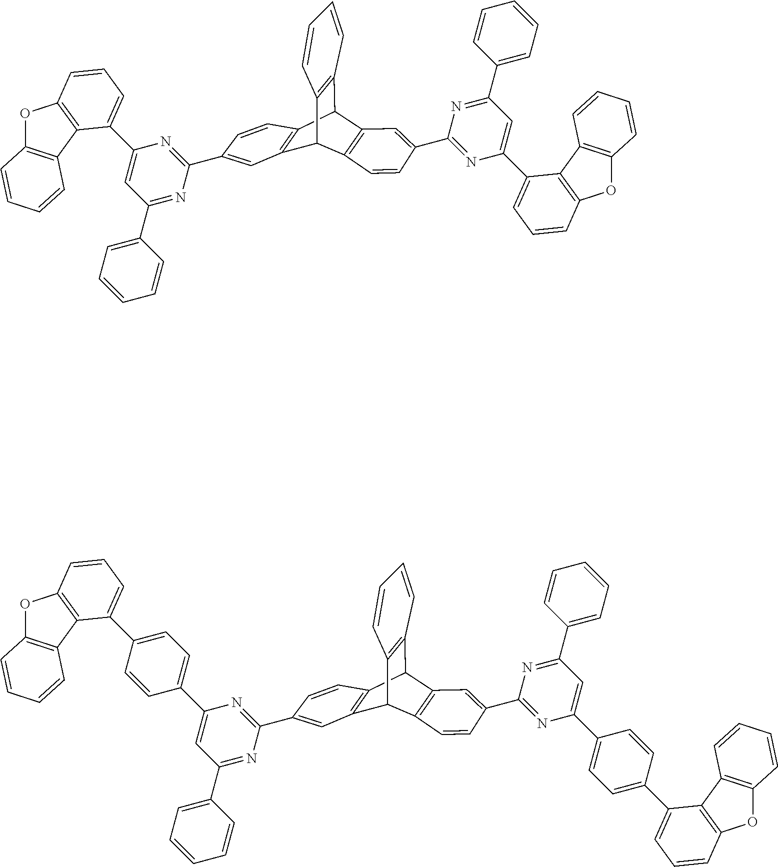

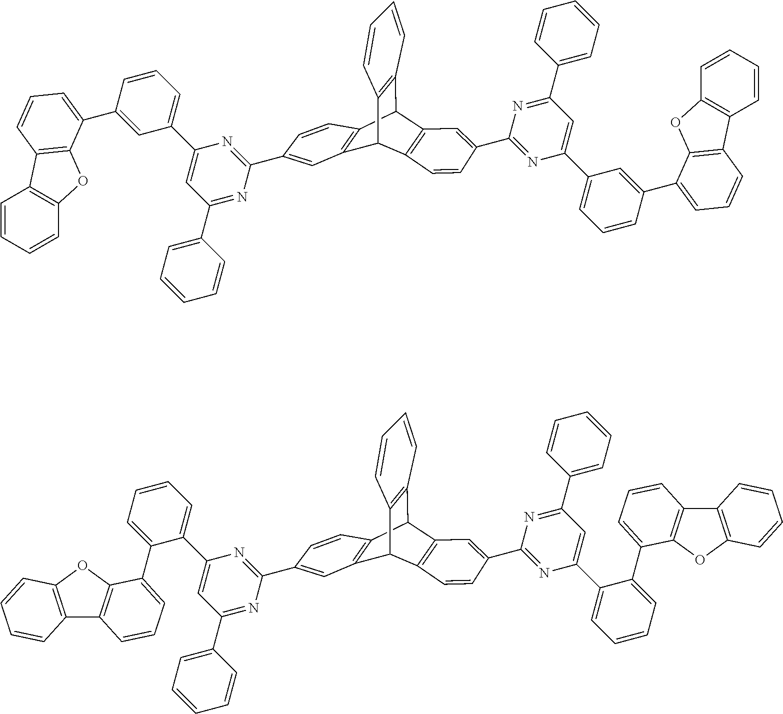 [SMILES: c1ccc(-c2cc(-c3cccc(-c4cccc5c4oc4ccccc45)c3)nc(-c3ccc4c(c3)C3c5ccccc5C4c4cc(-c5nc(-c6ccccc6)cc(-c6cccc(-c7cccc8c7oc7ccccc78)c6)n5)ccc43)n2)cc1 ; c1ccc(-c2cc(-c3ccccc3-c3cccc4c3oc3ccccc34)nc(-c3ccc4c(c3)C3c5ccccc5C4c4cc(-c5nc(-c6ccccc6)cc(-c6ccccc6-c6cccc7c6oc6ccccc67)n5)ccc43)n2)cc1]

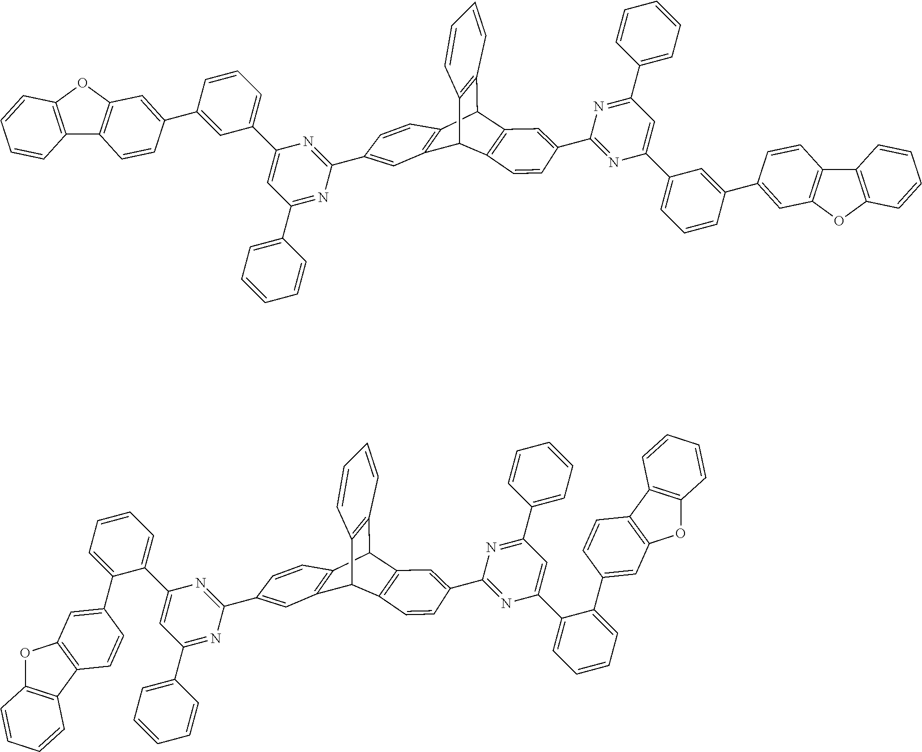 [SMILES: c1ccc(-c2cc(-c3cccc(-c4ccc5c(c4)oc4ccccc45)c3)nc(-c3ccc4c(c3)C3c5ccccc5C4c4cc(-c5nc(-c6ccccc6)cc(-c6cccc(-c7ccc8c(c7)oc7ccccc78)c6)n5)ccc43)n2)cc1 ; c1ccc(-c2cc(-c3ccccc3-c3ccc4c(c3)oc3ccccc34)nc(-c3ccc4c(c3)C3c5ccccc5C4c4cc(-c5nc(-c6ccccc6)cc(-c6ccccc6-c6ccc7c(c6)oc6ccccc67)n5)ccc43)n2)cc1]

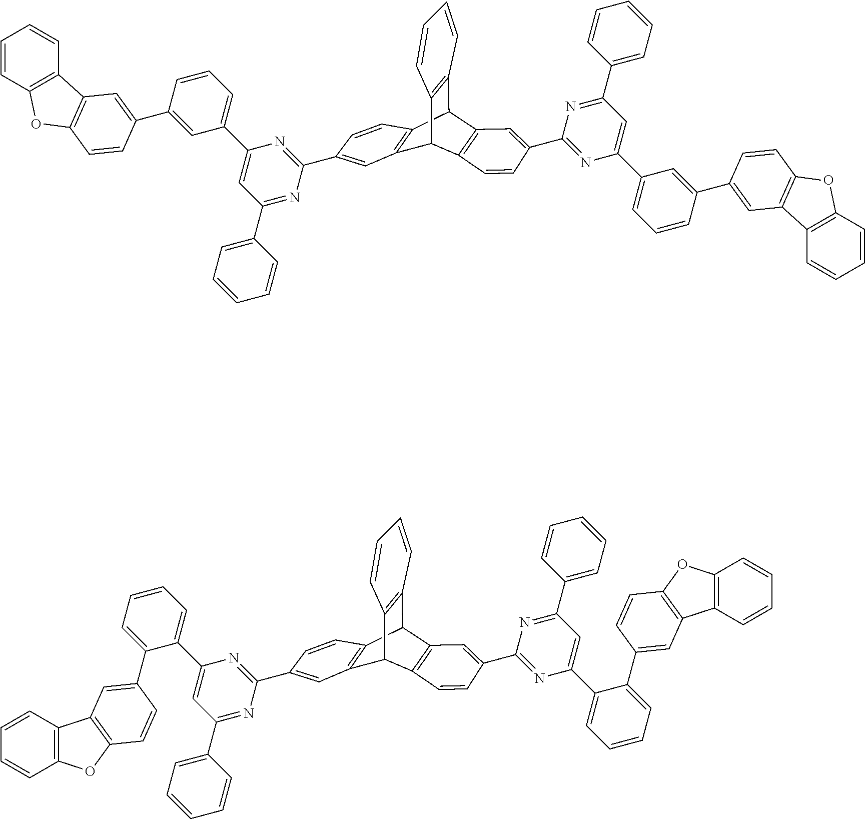 [SMILES: c1ccc(-c2cc(-c3cccc(-c4ccc5oc6ccccc6c5c4)c3)nc(-c3ccc4c(c3)C3c5ccccc5C4c4cc(-c5nc(-c6ccccc6)cc(-c6cccc(-c7ccc8oc9ccccc9c8c7)c6)n5)ccc43)n2)cc1 ; c1ccc(-c2cc(-c3ccccc3-c3ccc4oc5ccccc5c4c3)nc(-c3ccc4c(c3)C3c5ccccc5C4c4cc(-c5nc(-c6ccccc6)cc(-c6ccccc6-c6ccc7oc8ccccc8c7c6)n5)ccc43)n2)cc1]

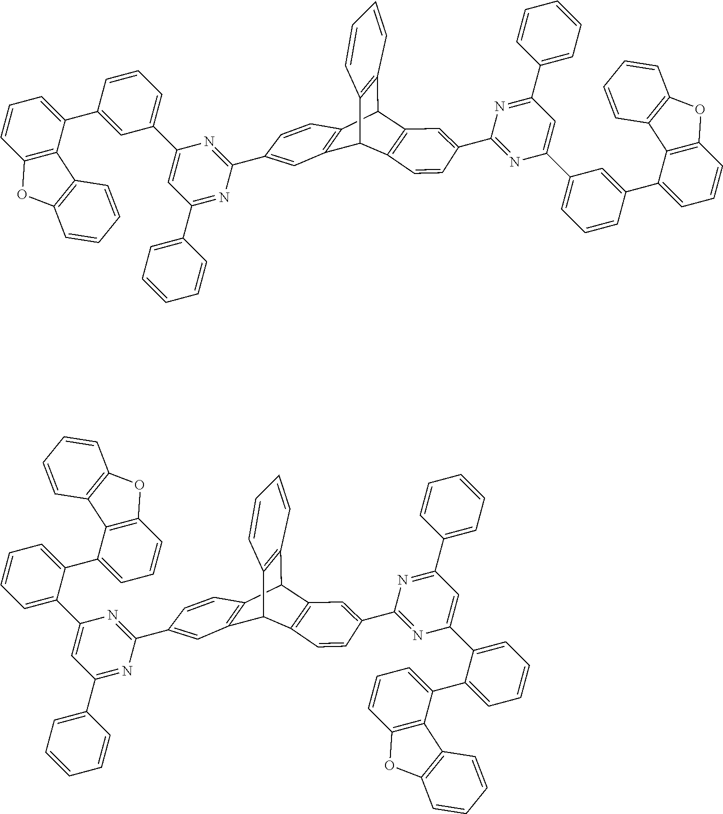 [SMILES: c1ccc(-c2cc(-c3cccc(-c4cccc5oc6ccccc6c45)c3)nc(-c3ccc4c(c3)C3c5ccccc5C4c4cc(-c5nc(-c6ccccc6)cc(-c6cccc(-c7cccc8oc9ccccc9c78)c6)n5)ccc43)n2)cc1 ; c1ccc(-c2cc(-c3ccccc3-c3cccc4oc5ccccc5c34)nc(-c3ccc4c(c3)C3c5ccccc5C4c4cc(-c5nc(-c6ccccc6)cc(-c6ccccc6-c6cccc7oc8ccccc8c67)n5)ccc43)n2)cc1]

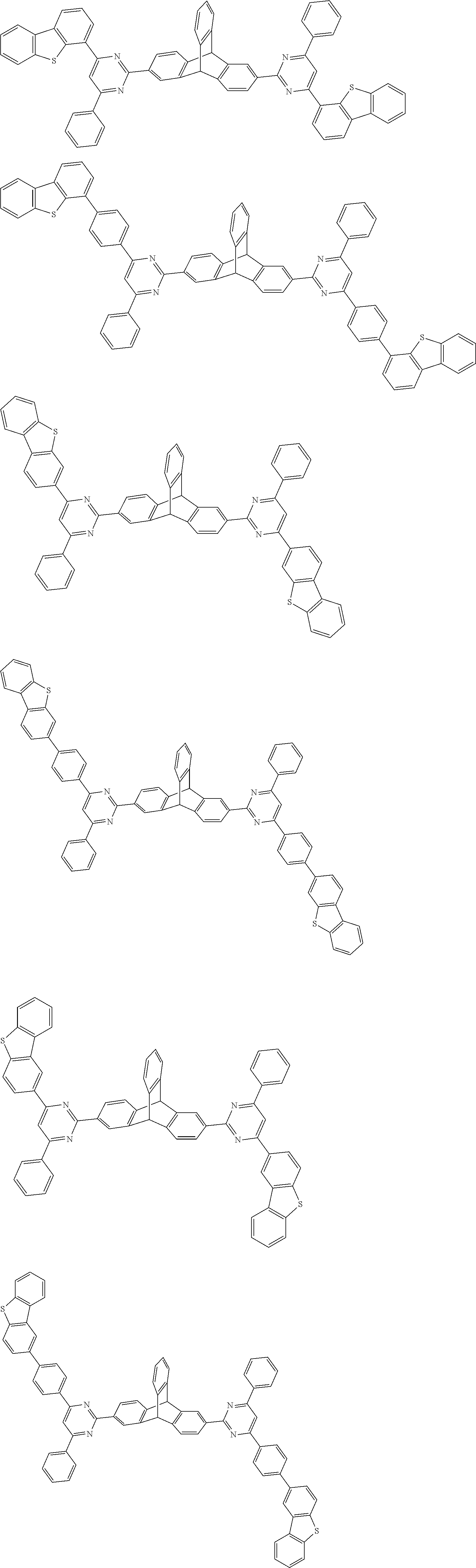

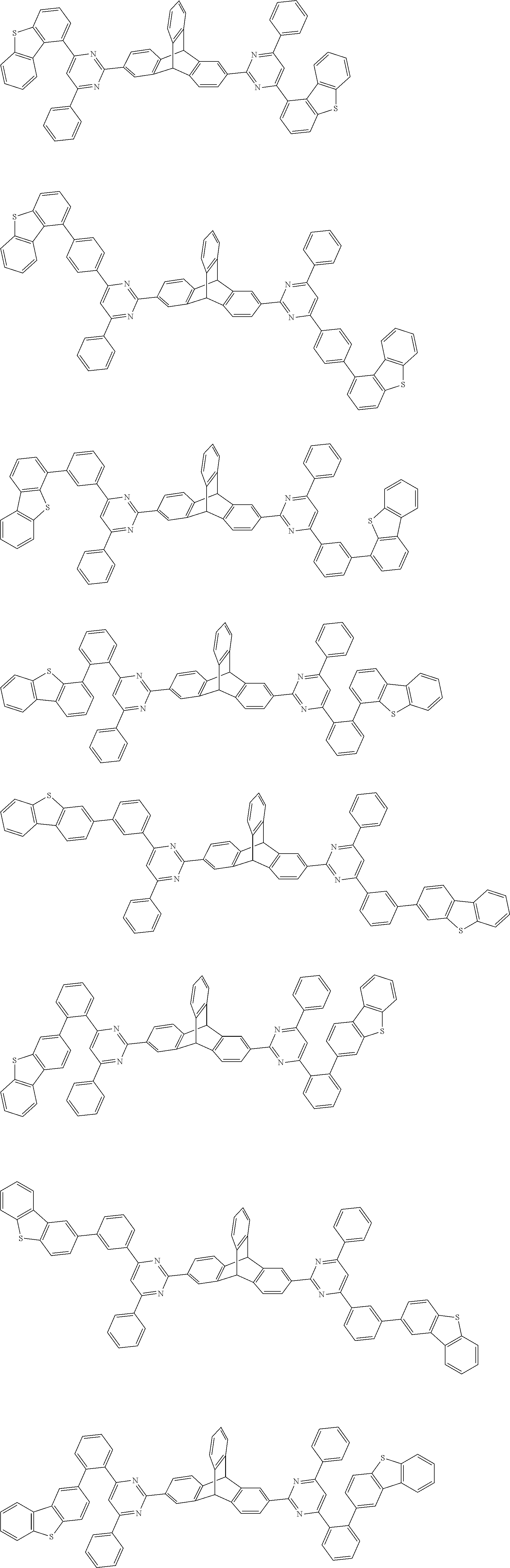

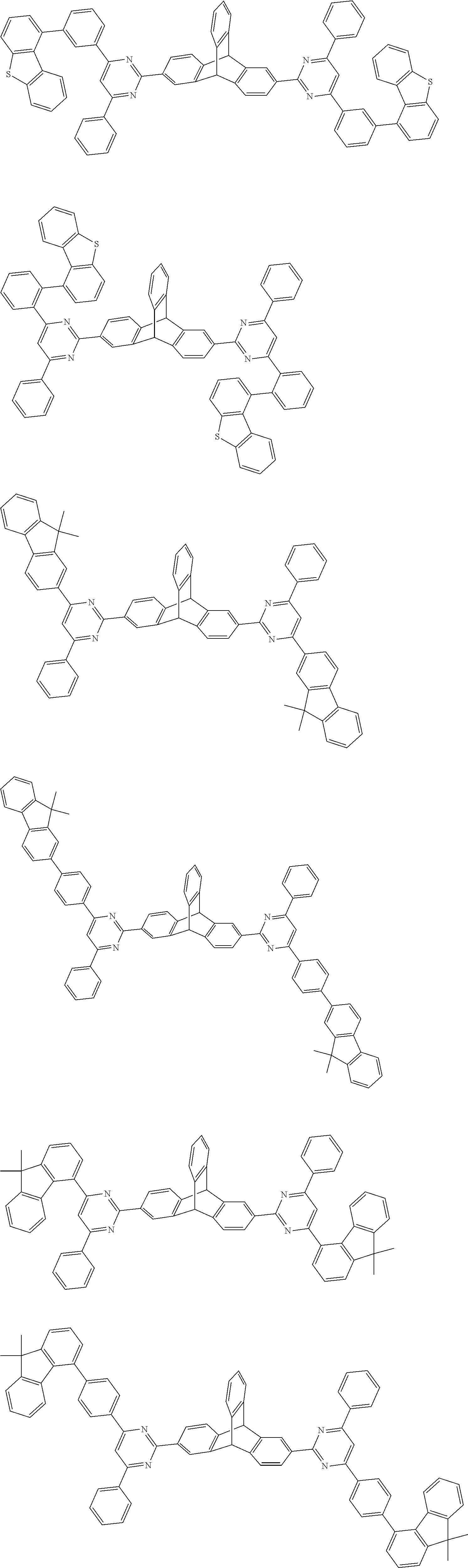

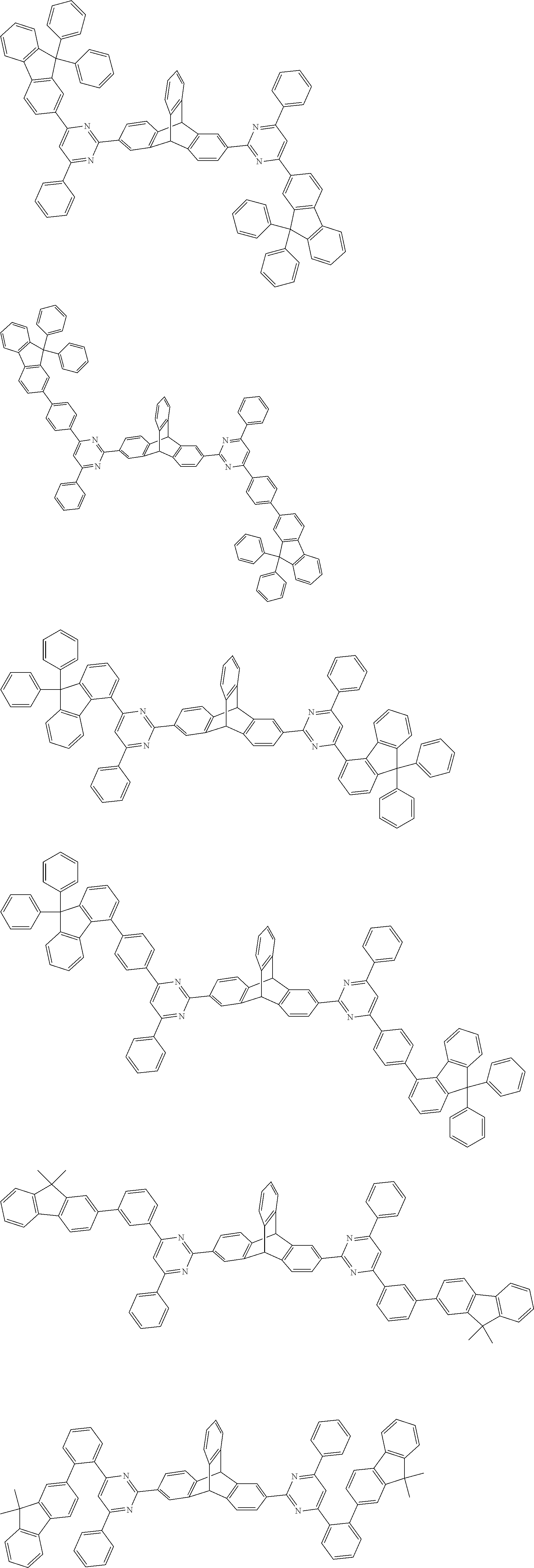

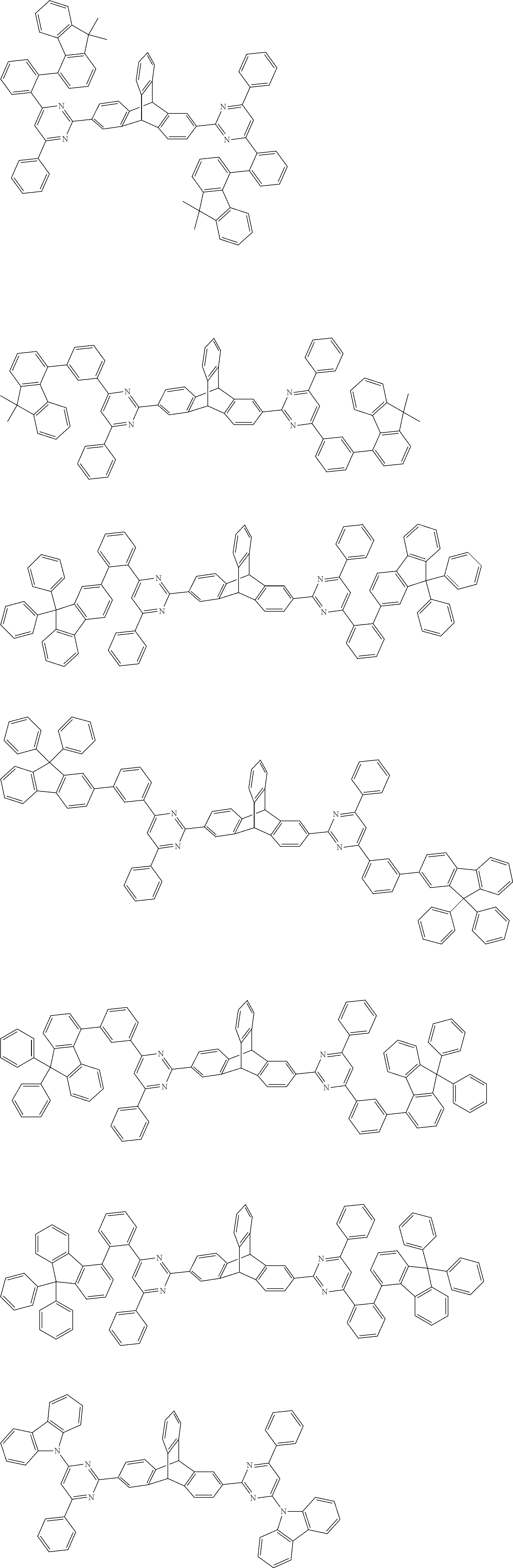

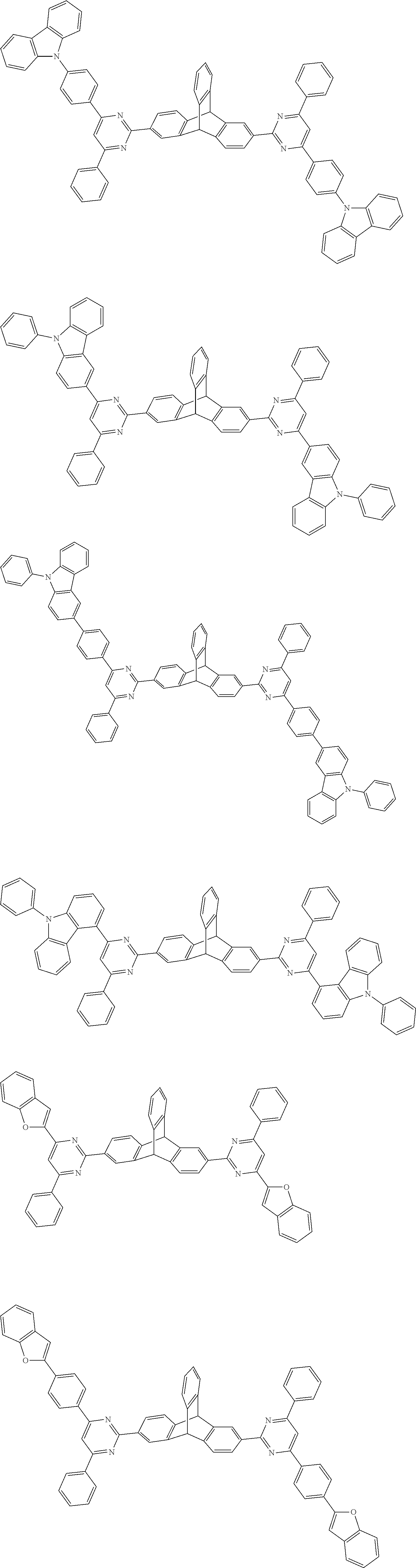

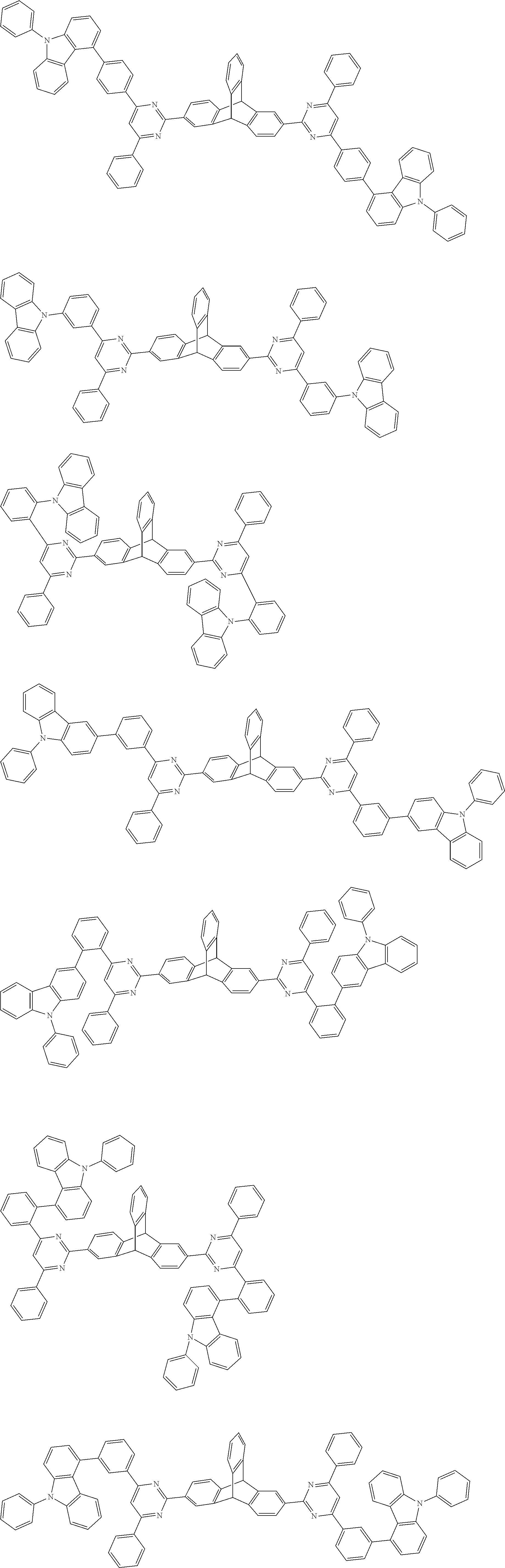

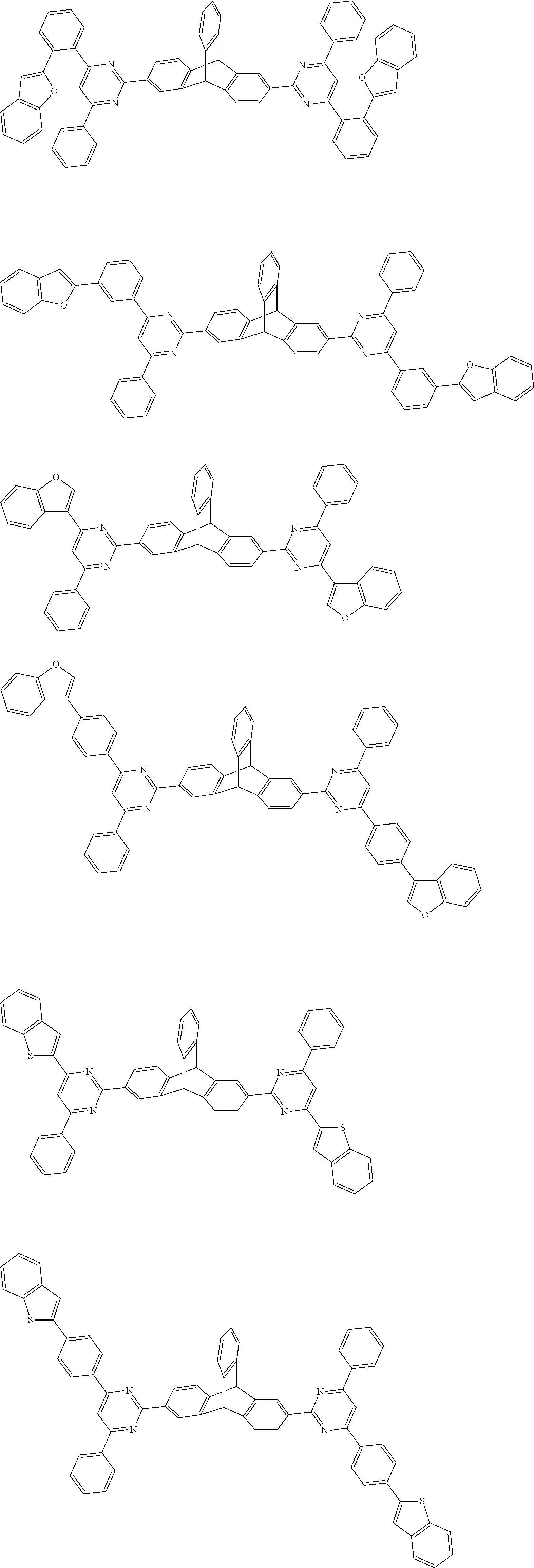

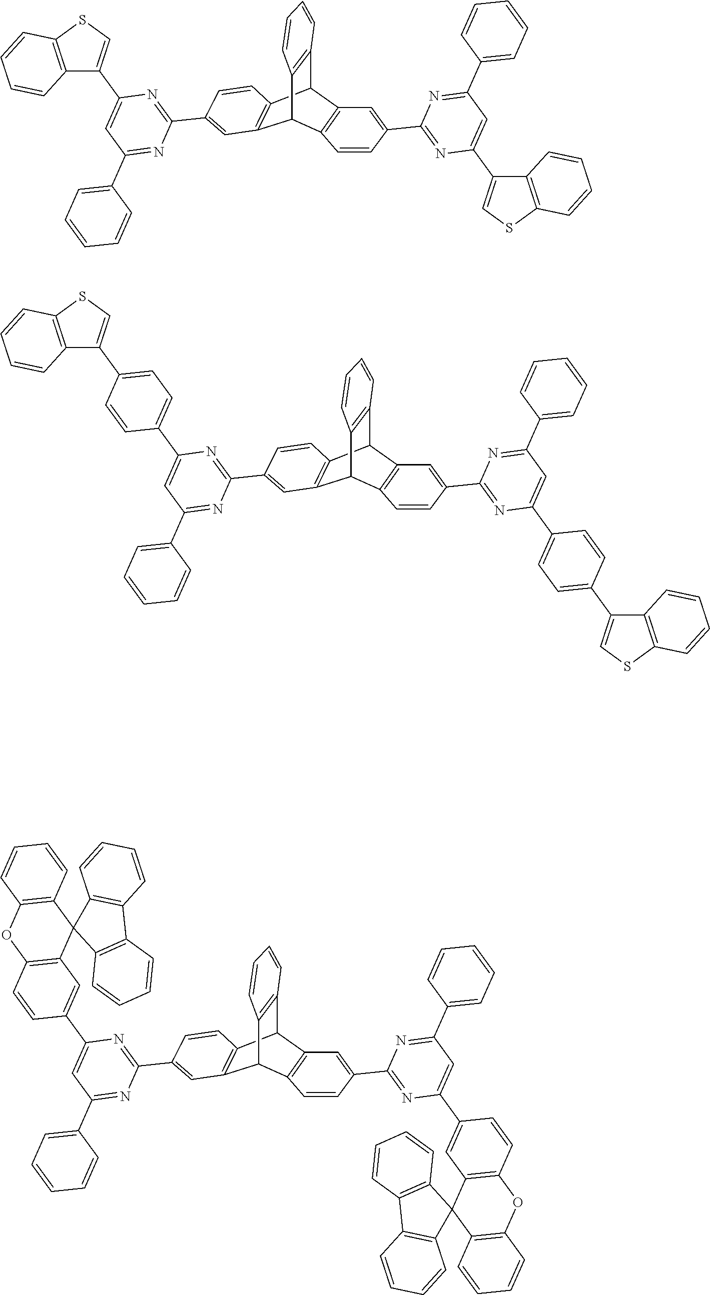 [SMILES: c1ccc(-c2cc(-c3csc4ccccc34)nc(-c3ccc4c(c3)C3c5ccccc5[C@H]4c4cc(-c5nc(-c6ccccc6)cc(-c6csc7ccccc67)n5)ccc43)n2)cc1 ; c1ccc(-c2cc(-c3ccc(-c4csc5ccccc45)cc3)nc(-c3ccc4c(c3)C3c5ccccc5[C@H]4c4cc(-c5nc(-c6ccccc6)cc(-c6ccc(-c7csc8ccccc78)cc6)n5)ccc43)n2)cc1 ; c1ccc(-c2cc(-c3ccc4c(c3)C3(c5ccccc5O4)c4ccccc4-c4ccccc43)nc(-c3ccc4c(c3)C3c5ccccc5[C@H]4c4cc(-c5nc(-c6ccccc6)cc(-c6ccc7c(c6)C6(c8ccccc8O7)c7ccccc7-c7ccccc76)n5)ccc43)n2)cc1]

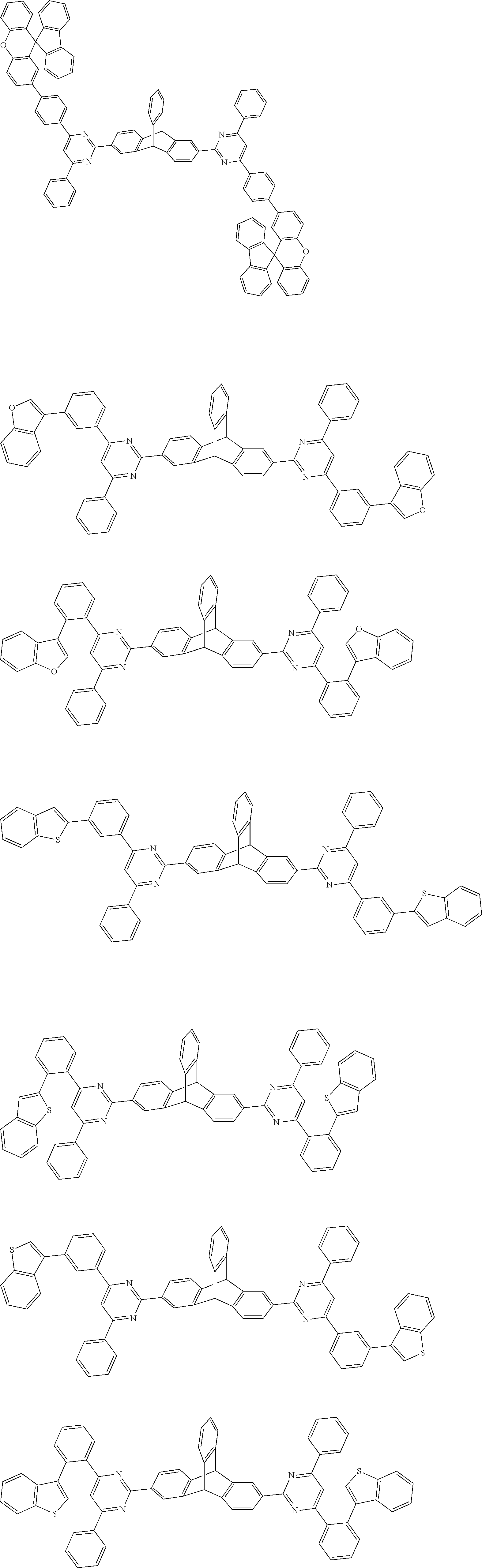

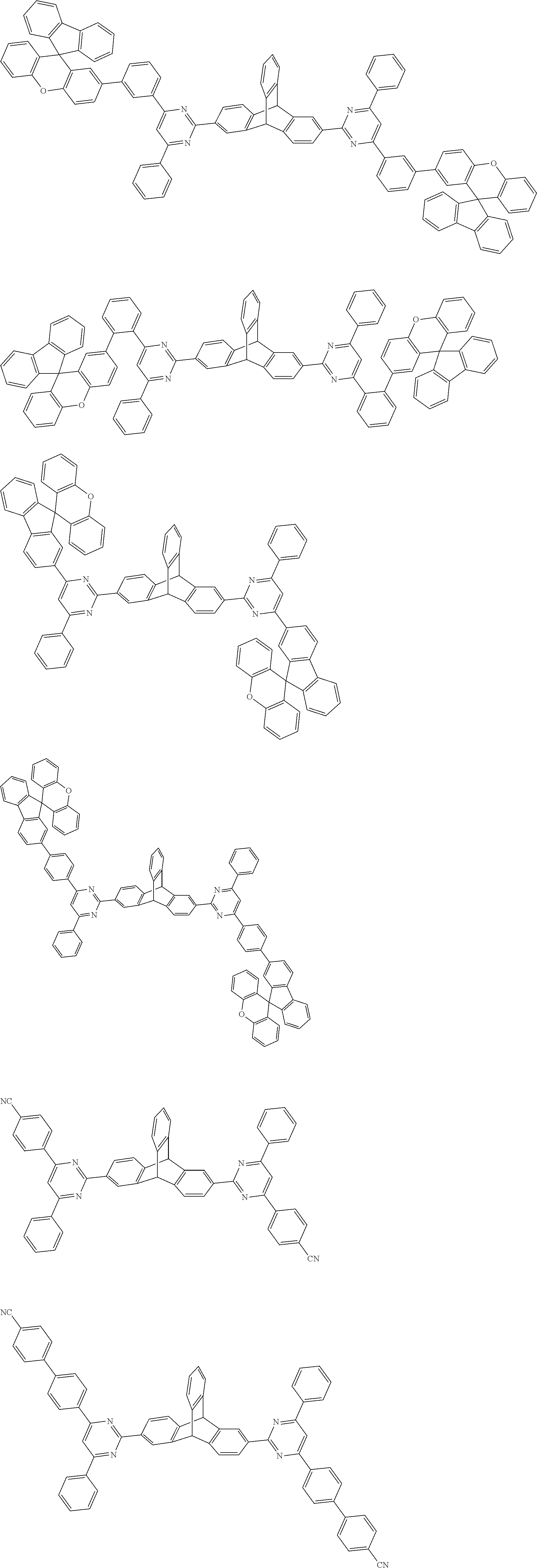

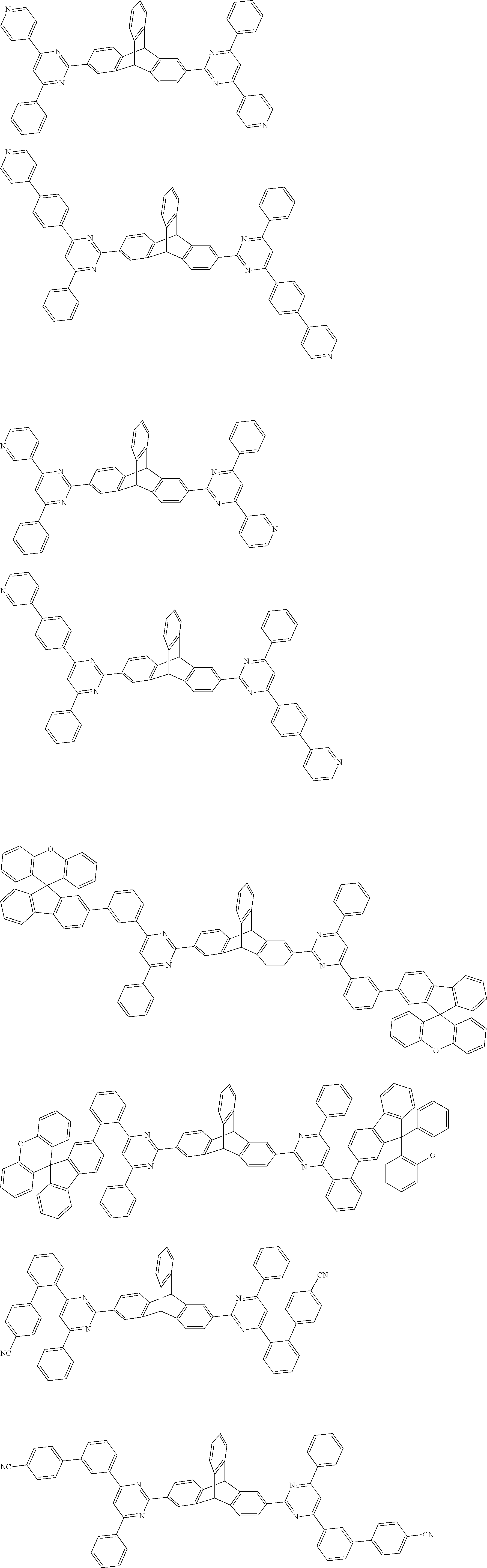

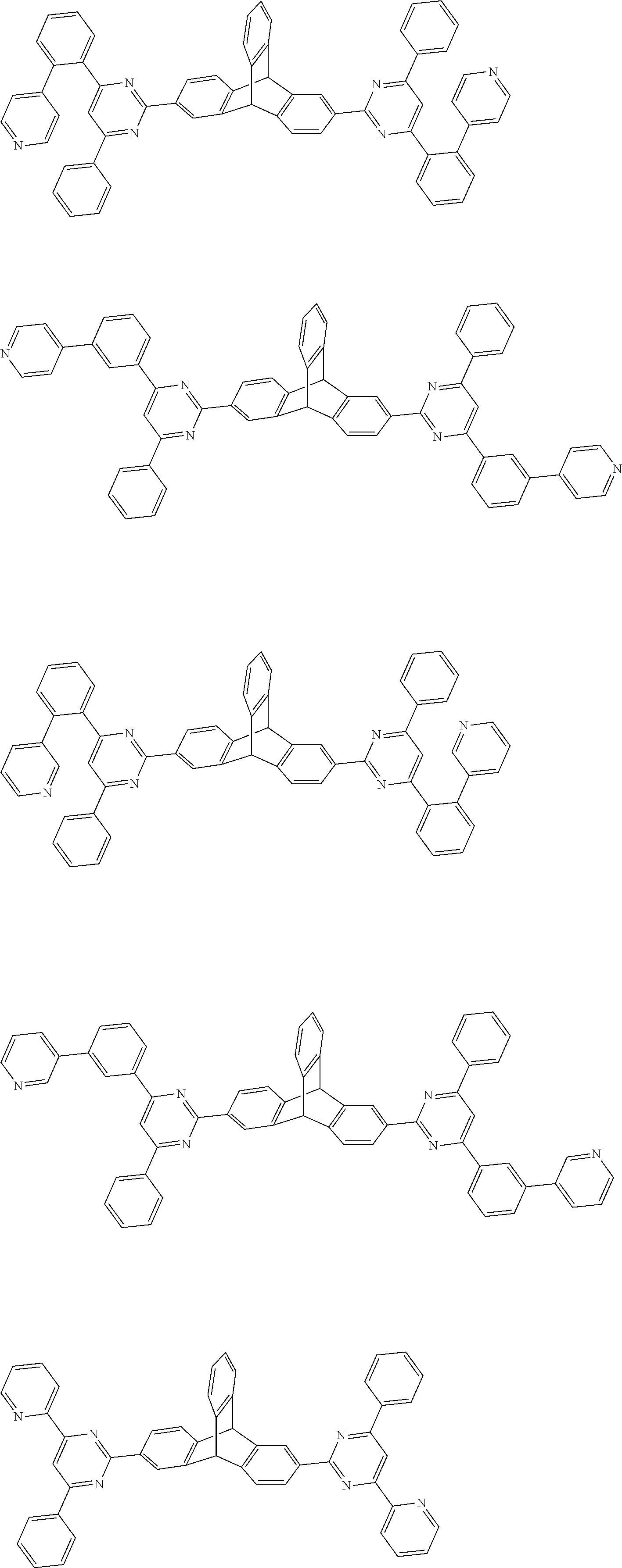

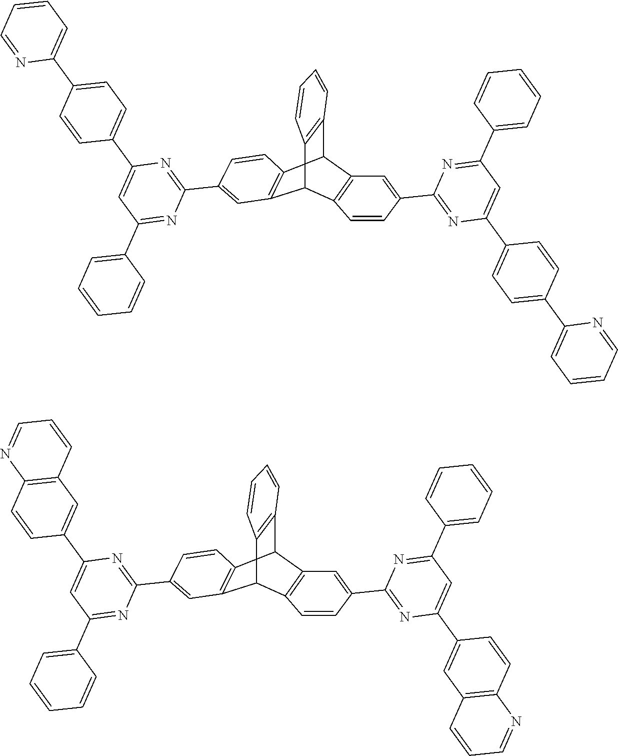 [SMILES: c1ccc(-c2cc(-c3ccc(-c4ccccn4)cc3)nc(-c3ccc4c(c3)C3c5ccccc5[C@H]4c4cc(-c5nc(-c6ccccc6)cc(-c6ccc(-c7ccccn7)cc6)n5)ccc43)n2)cc1 ; c1ccc(-c2cc(-c3ccc4ncccc4c3)nc(-c3ccc4c(c3)C3c5ccccc5[C@H]4c4cc(-c5nc(-c6ccccc6)cc(-c6ccc7ncccc7c6)n5)ccc43)n2)cc1]

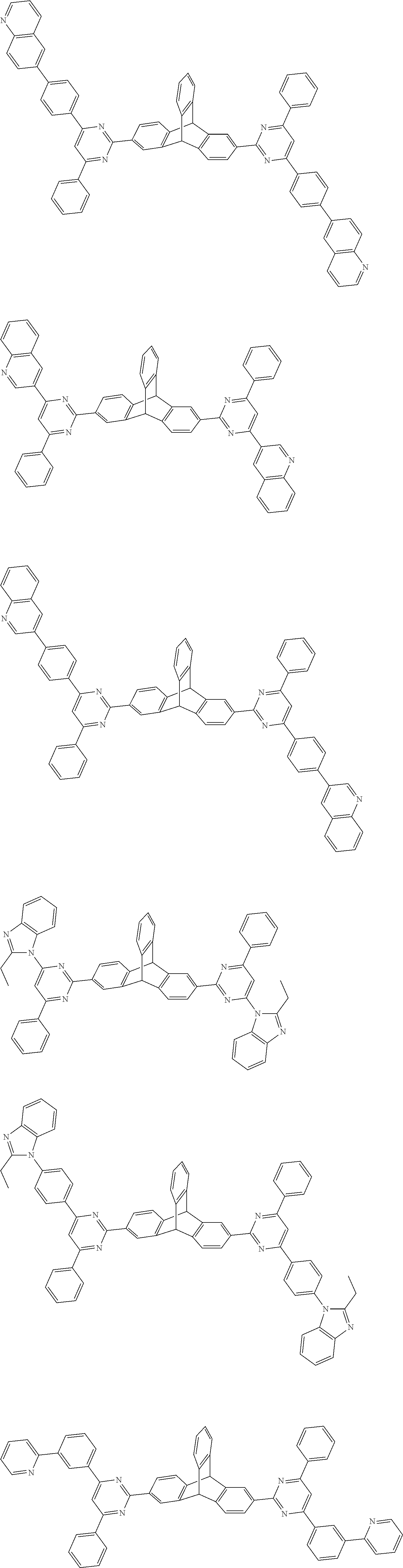

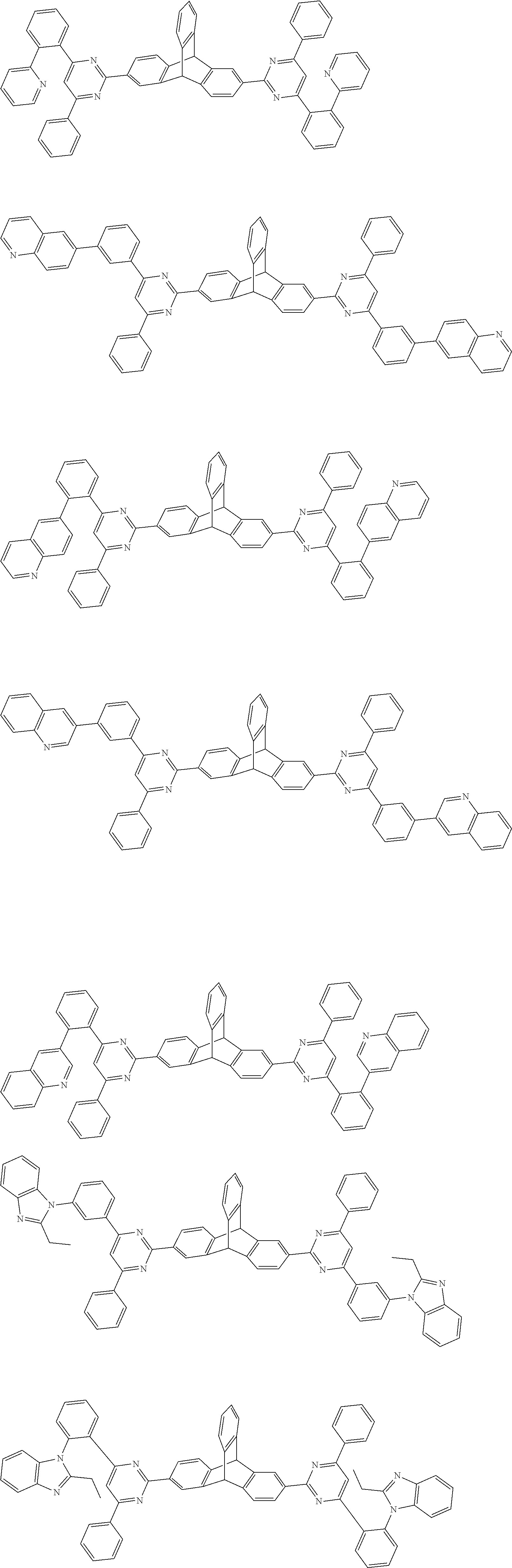

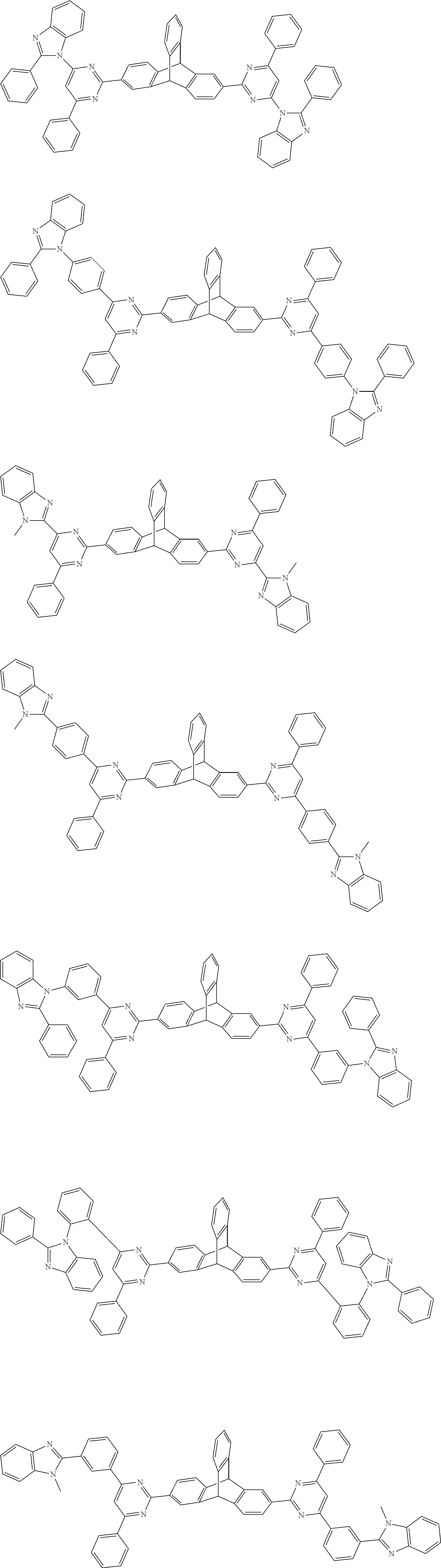

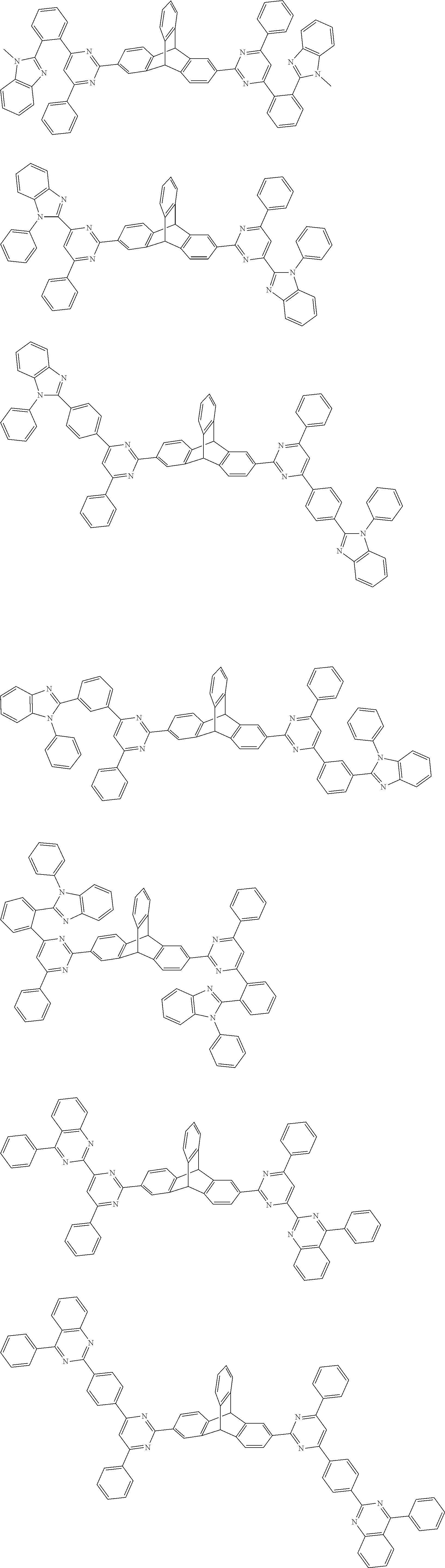

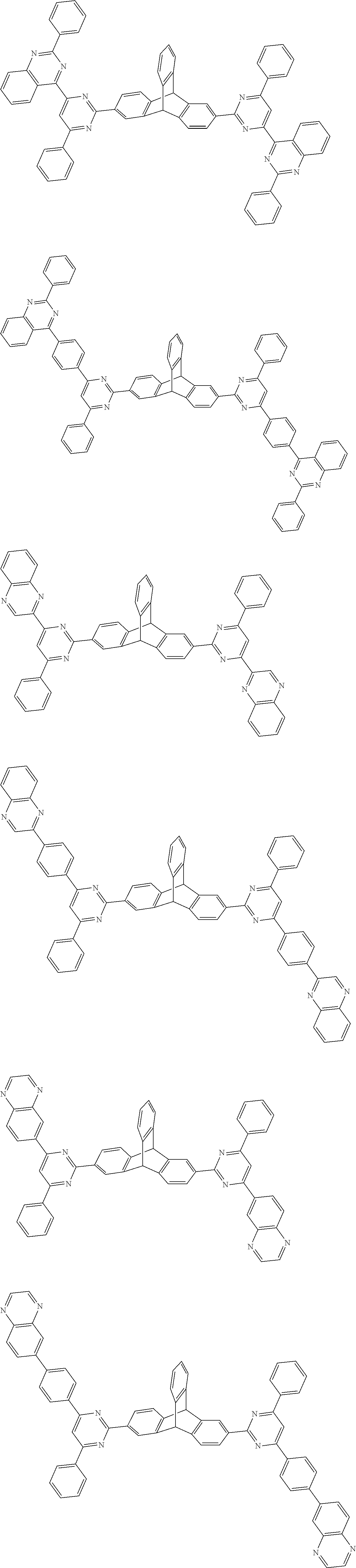

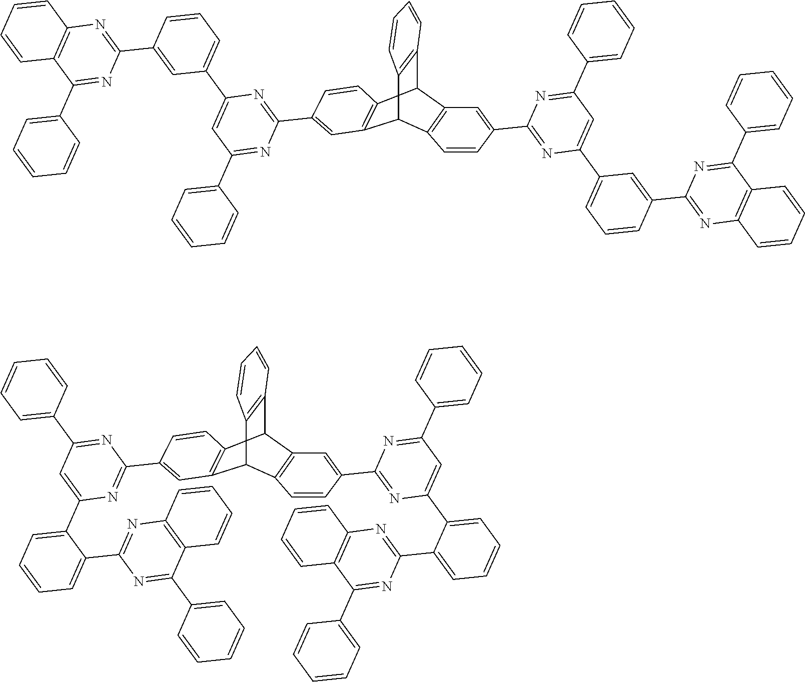 [SMILES: c1ccc(-c2cc(-c3cccc(-c4nc(-c5ccccc5)c5ccccc5n4)c3)nc(-c3ccc4c(c3)C3c5ccccc5C4c4cc(-c5nc(-c6ccccc6)cc(-c6cccc(-c7nc(-c8ccccc8)c8ccccc8n7)c6)n5)ccc43)n2)cc1 ; c1ccc(-c2cc(-c3ccccc3-c3nc(-c4ccccc4)c4ccccc4n3)nc(-c3ccc4c(c3)C3c5ccccc5C4c4cc(-c5nc(-c6ccccc6)cc(-c6ccccc6-c6nc(-c7ccccc7)c7ccccc7n6)n5)ccc43)n2)cc1]

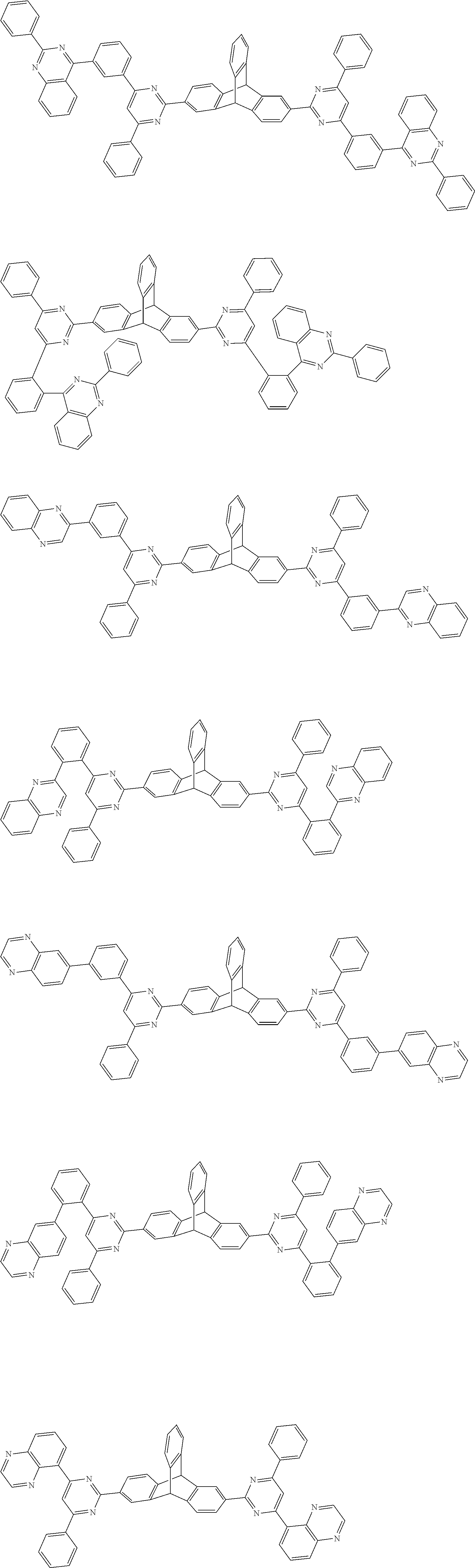

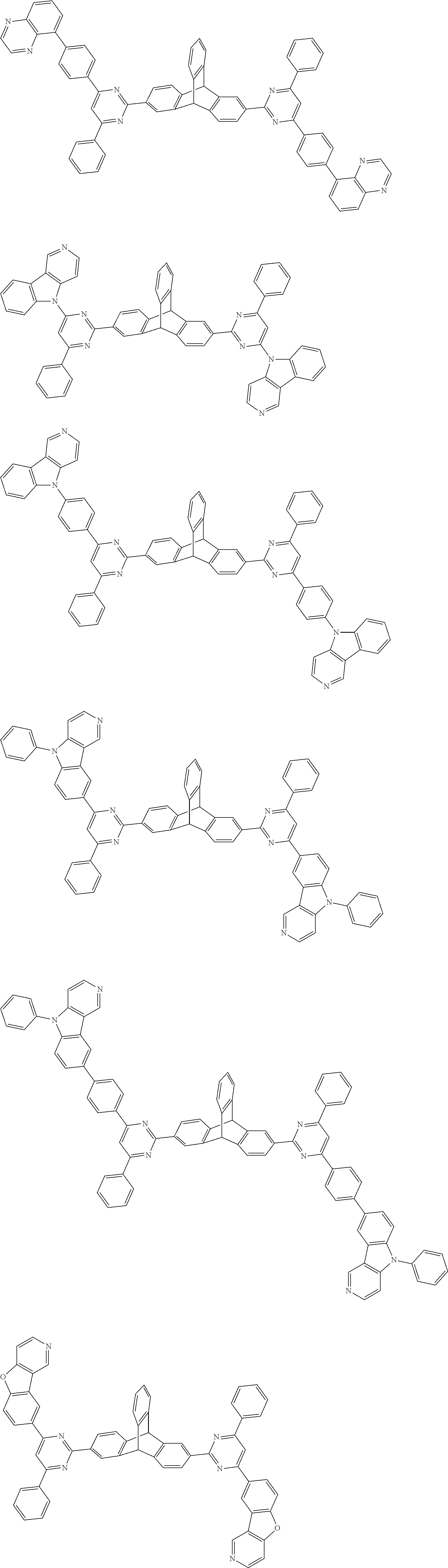

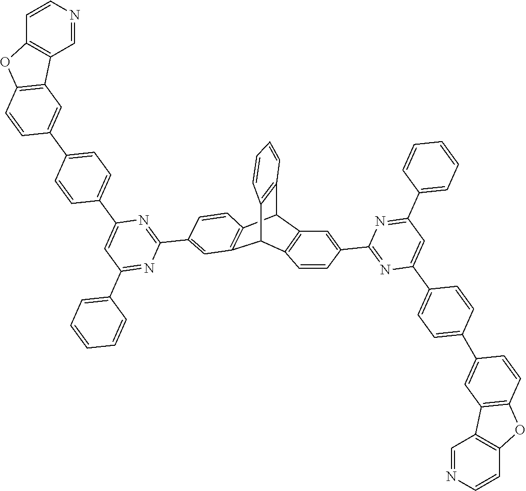 [SMILES: c1ccc(-c2cc(-c3ccc(-c4ccc5oc6ccncc6c5c4)cc3)nc(-c3ccc4c(c3)C3c5ccccc5C4c4cc(-c5nc(-c6ccccc6)cc(-c6ccc(-c7ccc8oc9ccncc9c8c7)cc6)n5)ccc43)n2)cc1]

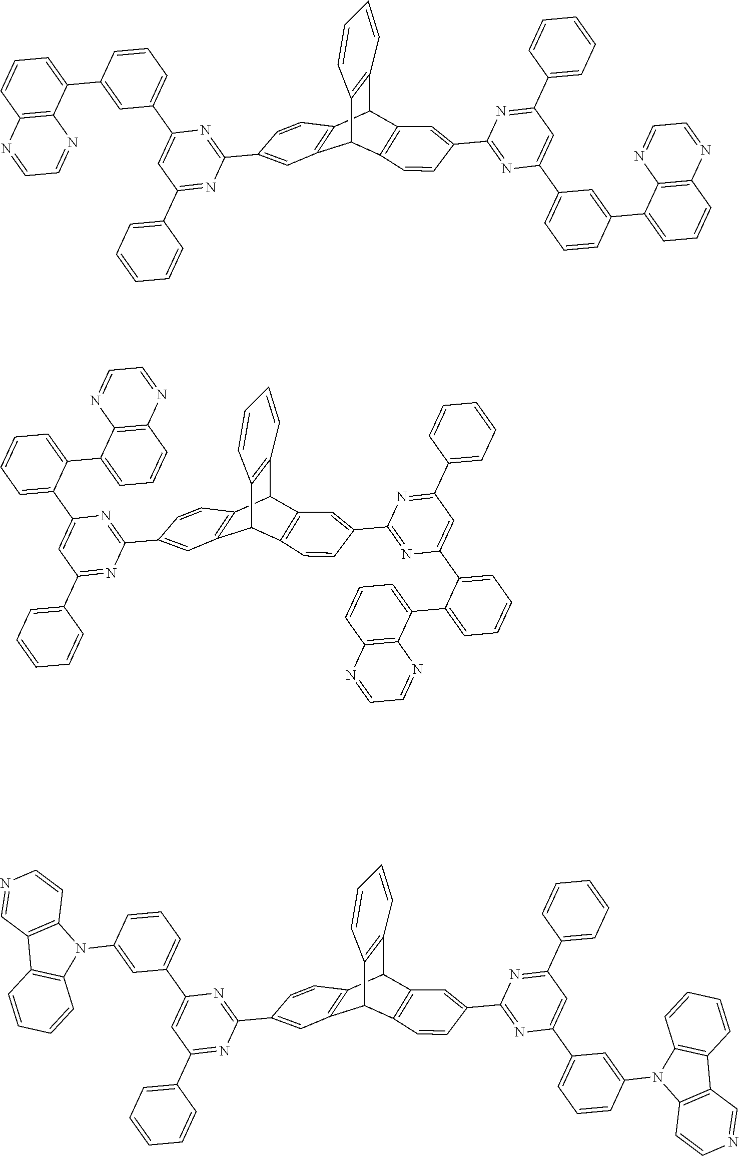 [SMILES: c1ccc(-c2cc(-c3cccc(-c4cccc5nccnc45)c3)nc(-c3ccc4c(c3)C3c5ccccc5C4c4cc(-c5nc(-c6ccccc6)cc(-c6cccc(-c7cccc8nccnc78)c6)n5)ccc43)n2)cc1 ; c1ccc(-c2cc(-c3ccccc3-c3cccc4nccnc34)nc(-c3ccc4c(c3)C3c5ccccc5C4c4cc(-c5nc(-c6ccccc6)cc(-c6ccccc6-c6cccc7nccnc67)n5)ccc43)n2)cc1 ; c1ccc(-c2cc(-c3cccc(-n4c5ccccc5c5cnccc54)c3)nc(-c3ccc4c(c3)C3c5ccccc5C4c4cc(-c5nc(-c6ccccc6)cc(-c6cccc(-n7c8ccccc8c8cnccc87)c6)n5)ccc43)n2)cc1]

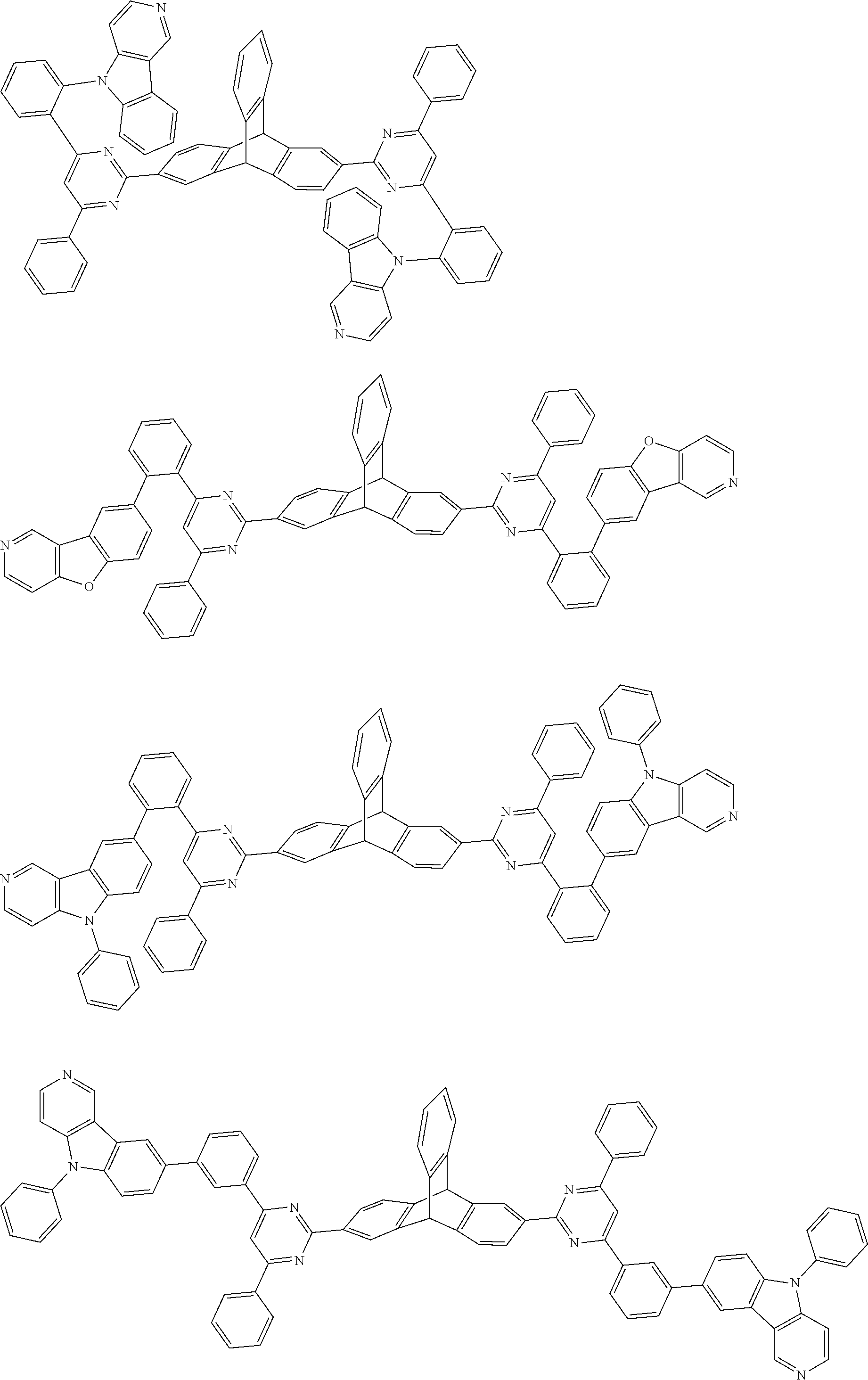

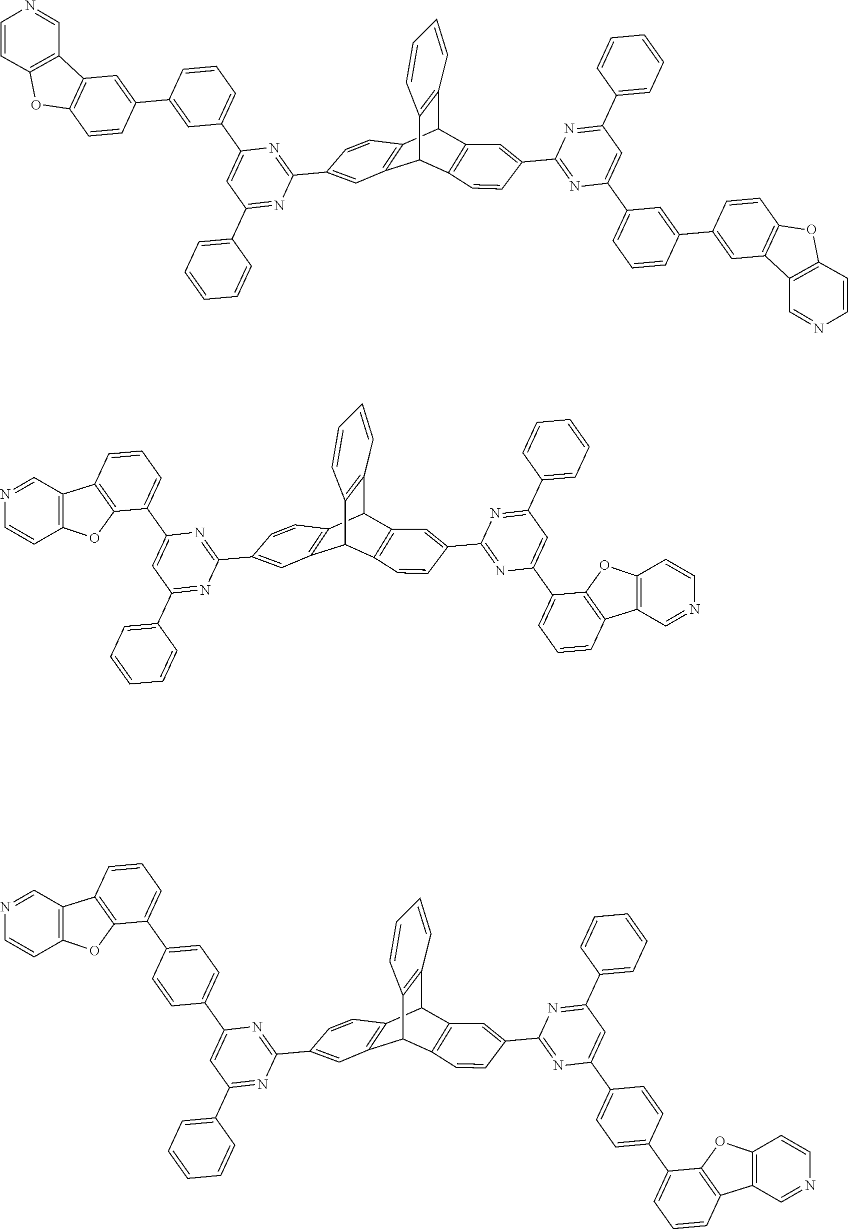 [SMILES: c1ccc(-c2cc(-c3cccc(-c4ccc5oc6ccncc6c5c4)c3)nc(-c3ccc4c(c3)C3c5ccccc5C4c4cc(-c5nc(-c6ccccc6)cc(-c6cccc(-c7ccc8oc9ccncc9c8c7)c6)n5)ccc43)n2)cc1 ; c1ccc(-c2cc(-c3cccc4c3oc3ccncc34)nc(-c3ccc4c(c3)C3c5ccccc5C4c4cc(-c5nc(-c6ccccc6)cc(-c6cccc7c6oc6ccncc67)n5)ccc43)n2)cc1 ; c1ccc(-c2cc(-c3ccc(-c4cccc5c4oc4ccncc45)cc3)nc(-c3ccc4c(c3)C3c5ccccc5C4c4cc(-c5nc(-c6ccccc6)cc(-c6ccc(-c7cccc8c7oc7ccncc78)cc6)n5)ccc43)n2)cc1]

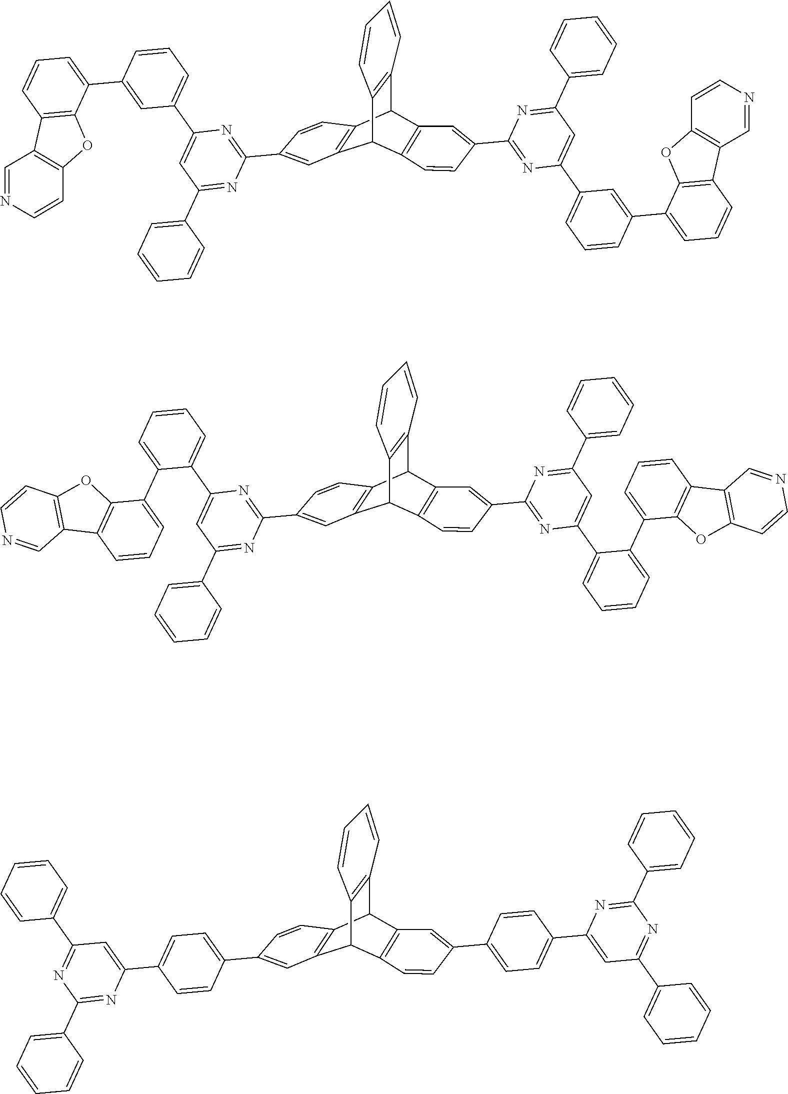 [SMILES: c1ccc(-c2cc(-c3cccc(-c4cccc5c4oc4ccncc45)c3)nc(-c3ccc4c(c3)C3c5ccccc5C4c4cc(-c5nc(-c6ccccc6)cc(-c6cccc(-c7cccc8c7oc7ccncc78)c6)n5)ccc43)n2)cc1 ; c1ccc(-c2cc(-c3ccccc3-c3cccc4c3oc3ccncc34)nc(-c3ccc4c(c3)C3c5ccccc5C4c4cc(-c5nc(-c6ccccc6)cc(-c6ccccc6-c6cccc7c6oc6ccncc67)n5)ccc43)n2)cc1 ; c1ccc(-c2cc(-c3ccc(-c4ccc5c(c4)C4c6ccccc6C5c5cc(-c6ccc(-c7cc(-c8ccccc8)nc(-c8ccccc8)n7)cc6)ccc54)cc3)nc(-c3ccccc3)n2)cc1]

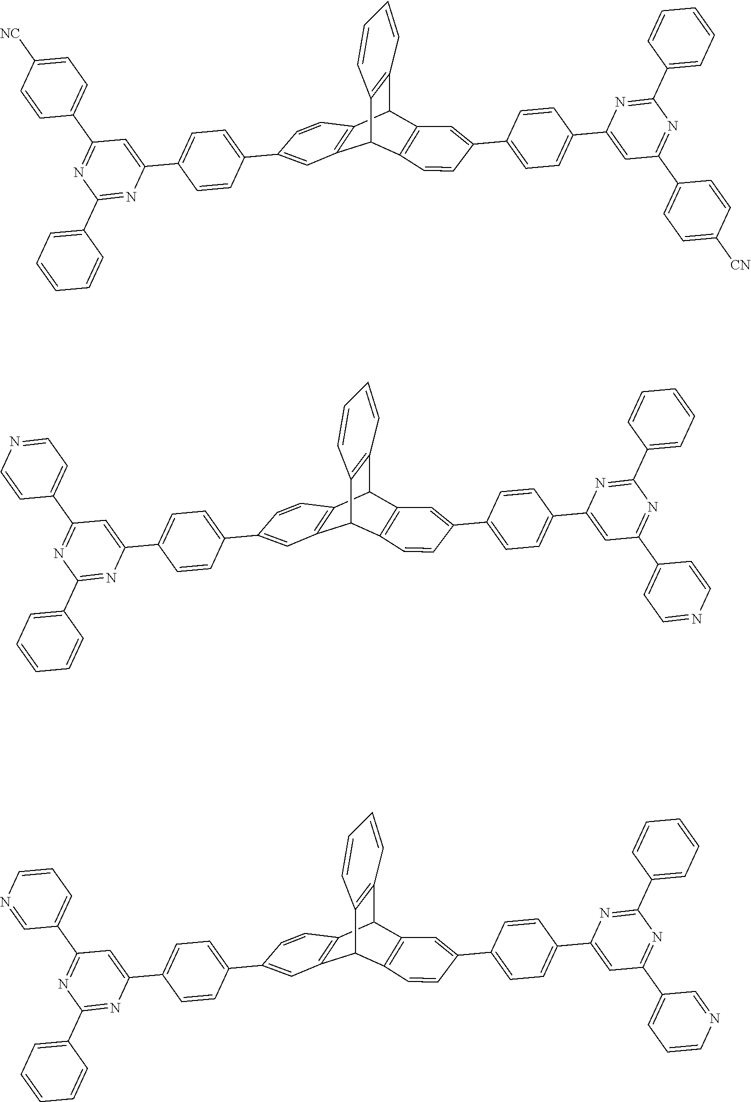 [SMILES: N#Cc1ccc(-c2cc(-c3ccc(-c4ccc5c(c4)C4c6ccccc6C5c5cc(-c6ccc(-c7cc(-c8ccc(C#N)cc8)nc(-c8ccccc8)n7)cc6)ccc54)cc3)nc(-c3ccccc3)n2)cc1 ; c1ccc(-c2nc(-c3ccncc3)cc(-c3ccc(-c4ccc5c(c4)C4c6ccccc6C5c5cc(-c6ccc(-c7cc(-c8ccncc8)nc(-c8ccccc8)n7)cc6)ccc54)cc3)n2)cc1 ; c1ccc(-c2nc(-c3ccc(-c4ccc5c(c4)C4c6ccccc6C5c5cc(-c6ccc(-c7cc(-c8cccnc8)nc(-c8ccccc8)n7)cc6)ccc54)cc3)cc(-c3cccnc3)n2)cc1]

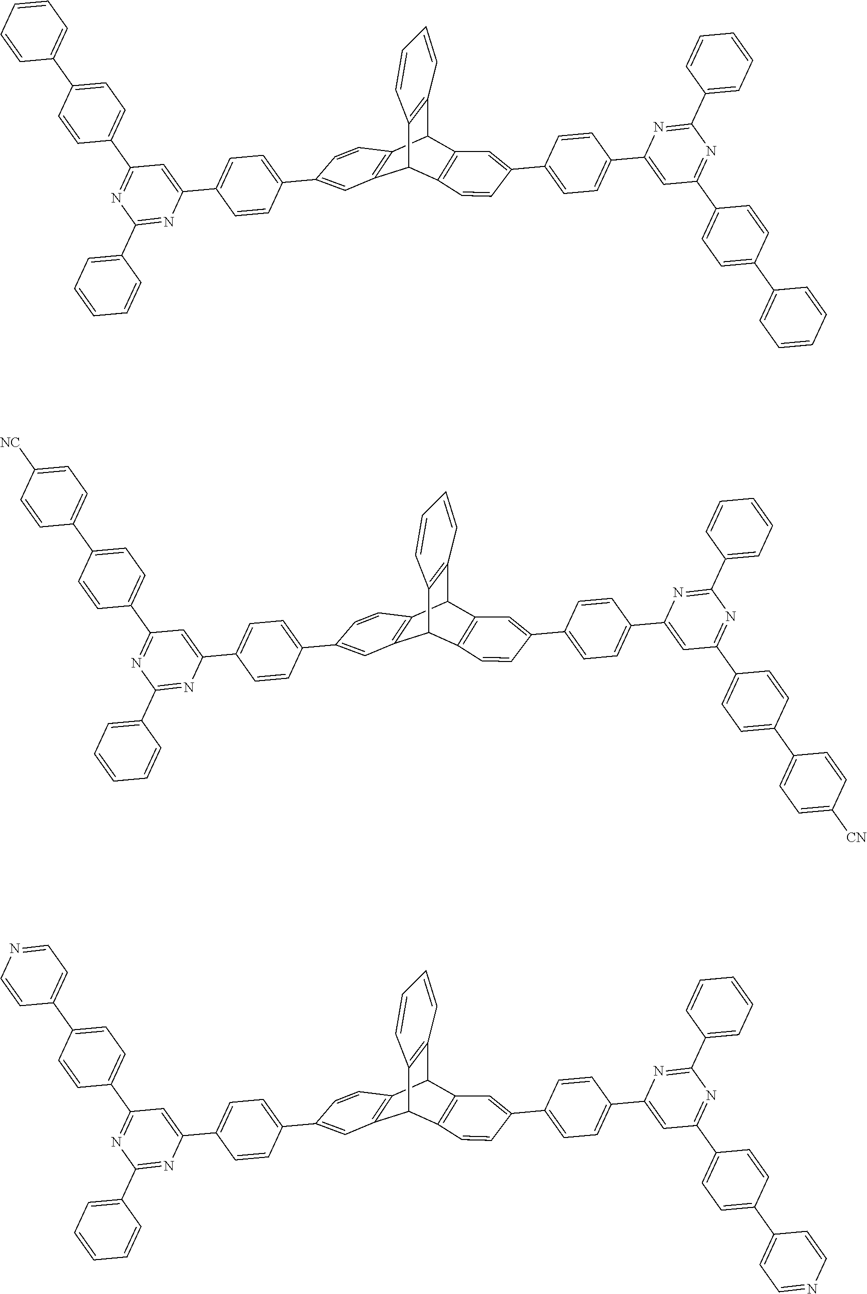 [SMILES: c1ccc(-c2ccc(-c3cc(-c4ccc(-c5ccc6c(c5)C5c7ccccc7C6c6cc(-c7ccc(-c8cc(-c9ccc(-c%10ccccc%10)cc9)nc(-c9ccccc9)n8)cc7)ccc65)cc4)nc(-c4ccccc4)n3)cc2)cc1 ; N#Cc1ccc(-c2ccc(-c3cc(-c4ccc(-c5ccc6c(c5)C5c7ccccc7C6c6cc(-c7ccc(-c8cc(-c9ccc(-c%10ccc(C#N)cc%10)cc9)nc(-c9ccccc9)n8)cc7)ccc65)cc4)nc(-c4ccccc4)n3)cc2)cc1 ; c1ccc(-c2nc(-c3ccc(-c4ccncc4)cc3)cc(-c3ccc(-c4ccc5c(c4)C4c6ccccc6C5c5cc(-c6ccc(-c7cc(-c8ccc(-c9ccncc9)cc8)nc(-c8ccccc8)n7)cc6)ccc54)cc3)n2)cc1]

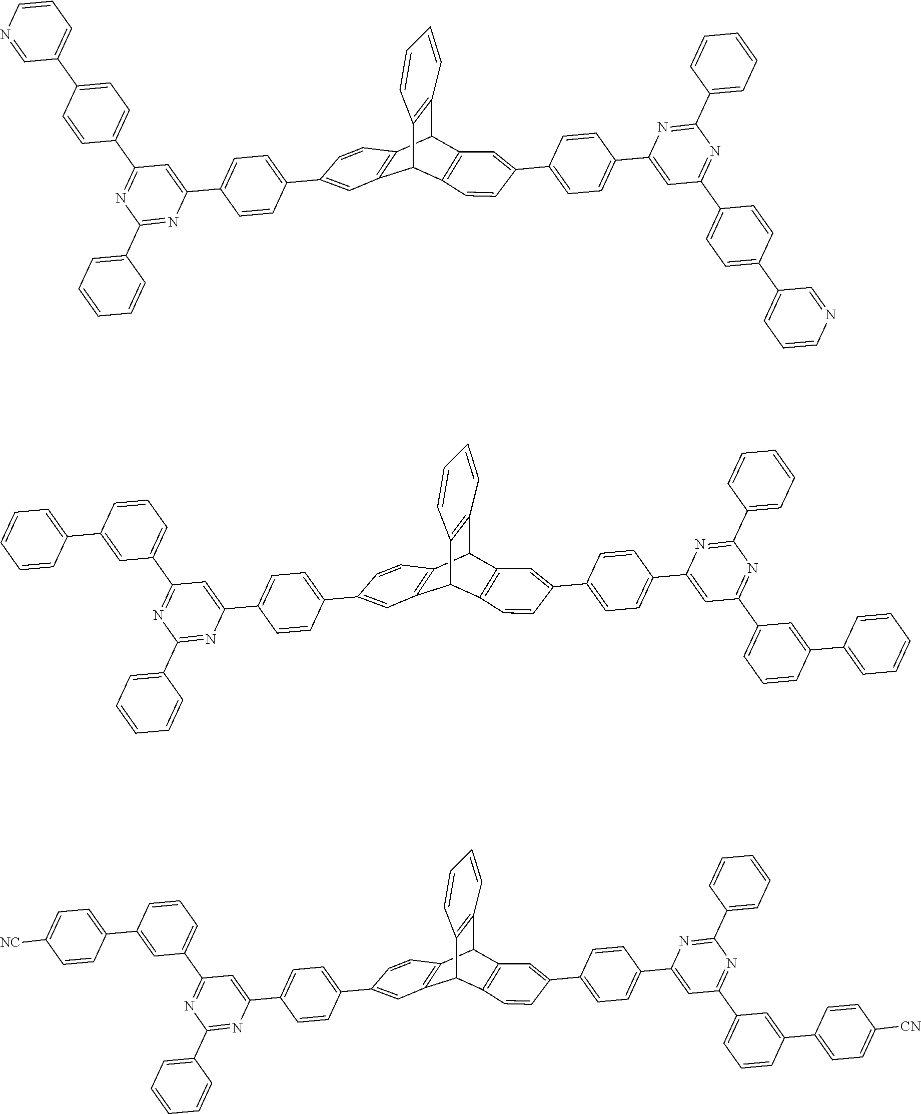 [SMILES: c1ccc(-c2nc(-c3ccc(-c4cccnc4)cc3)cc(-c3ccc(-c4ccc5c(c4)C4c6ccccc6C5c5cc(-c6ccc(-c7cc(-c8ccc(-c9cccnc9)cc8)nc(-c8ccccc8)n7)cc6)ccc54)cc3)n2)cc1 ; c1ccc(-c2cccc(-c3cc(-c4ccc(-c5ccc6c(c5)C5c7ccccc7C6c6cc(-c7ccc(-c8cc(-c9cccc(-c%10ccccc%10)c9)nc(-c9ccccc9)n8)cc7)ccc65)cc4)nc(-c4ccccc4)n3)c2)cc1 ; N#Cc1ccc(-c2cccc(-c3cc(-c4ccc(-c5ccc6c(c5)C5c7ccccc7C6c6cc(-c7ccc(-c8cc(-c9cccc(-c%10ccc(C#N)cc%10)c9)nc(-c9ccccc9)n8)cc7)ccc65)cc4)nc(-c4ccccc4)n3)c2)cc1]

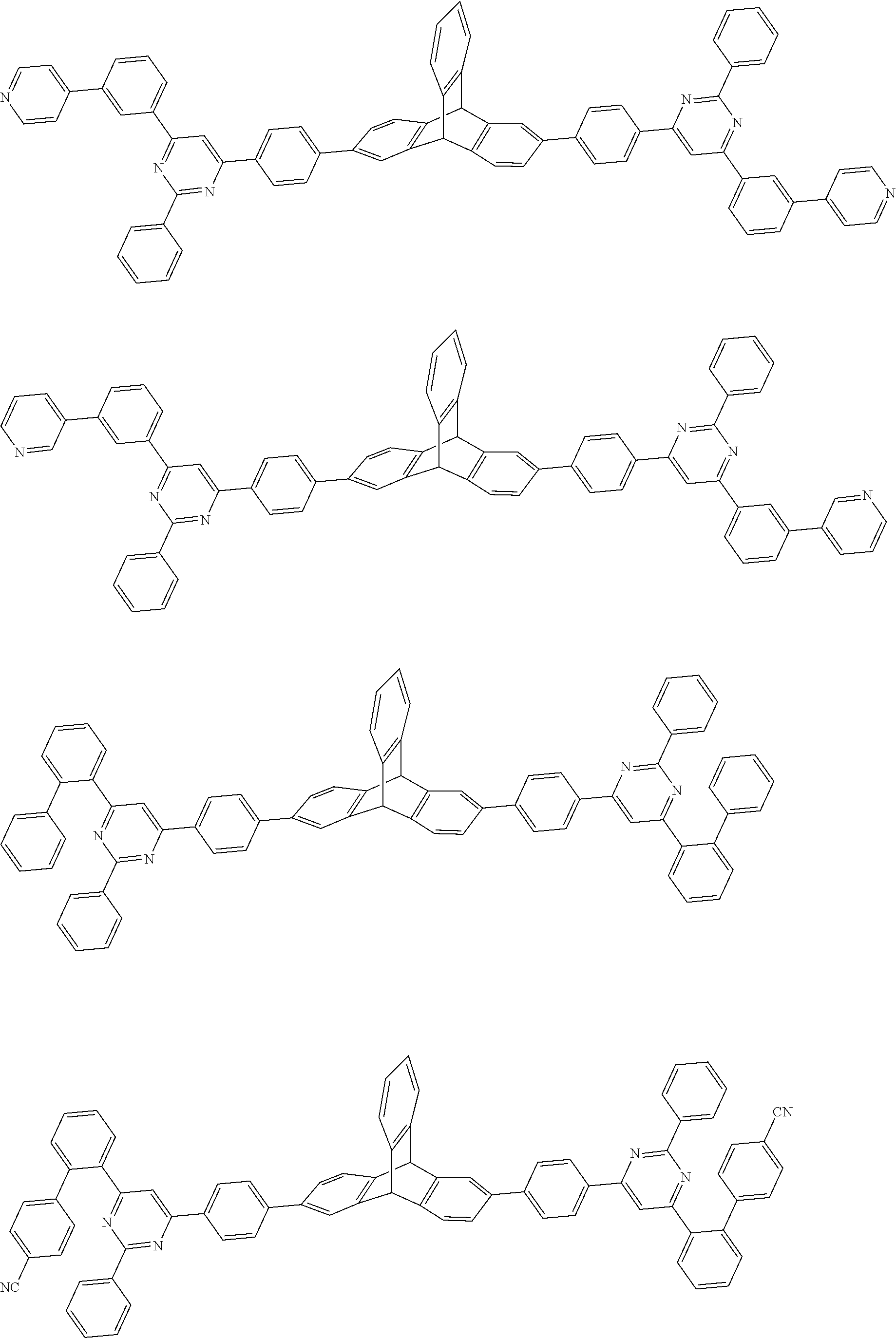

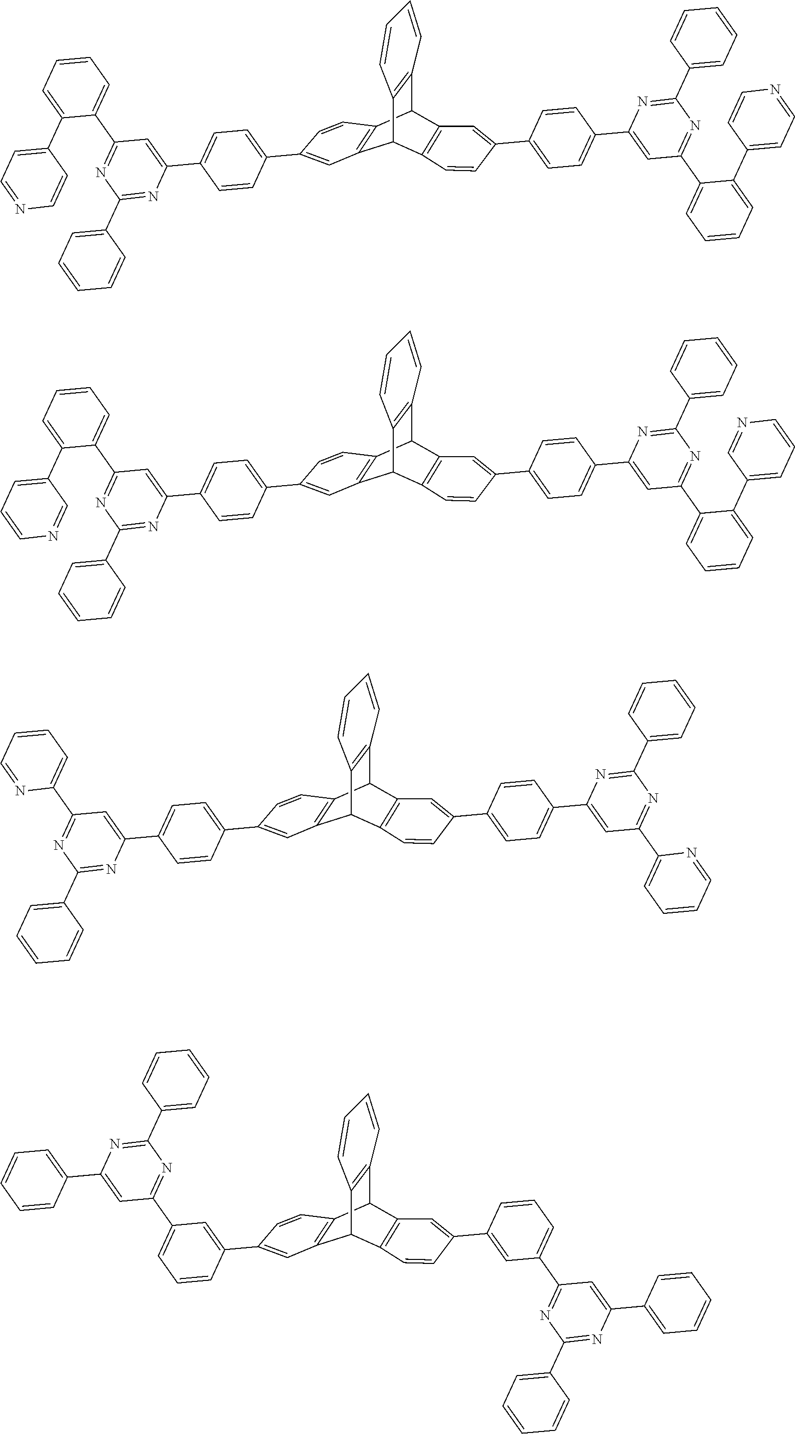

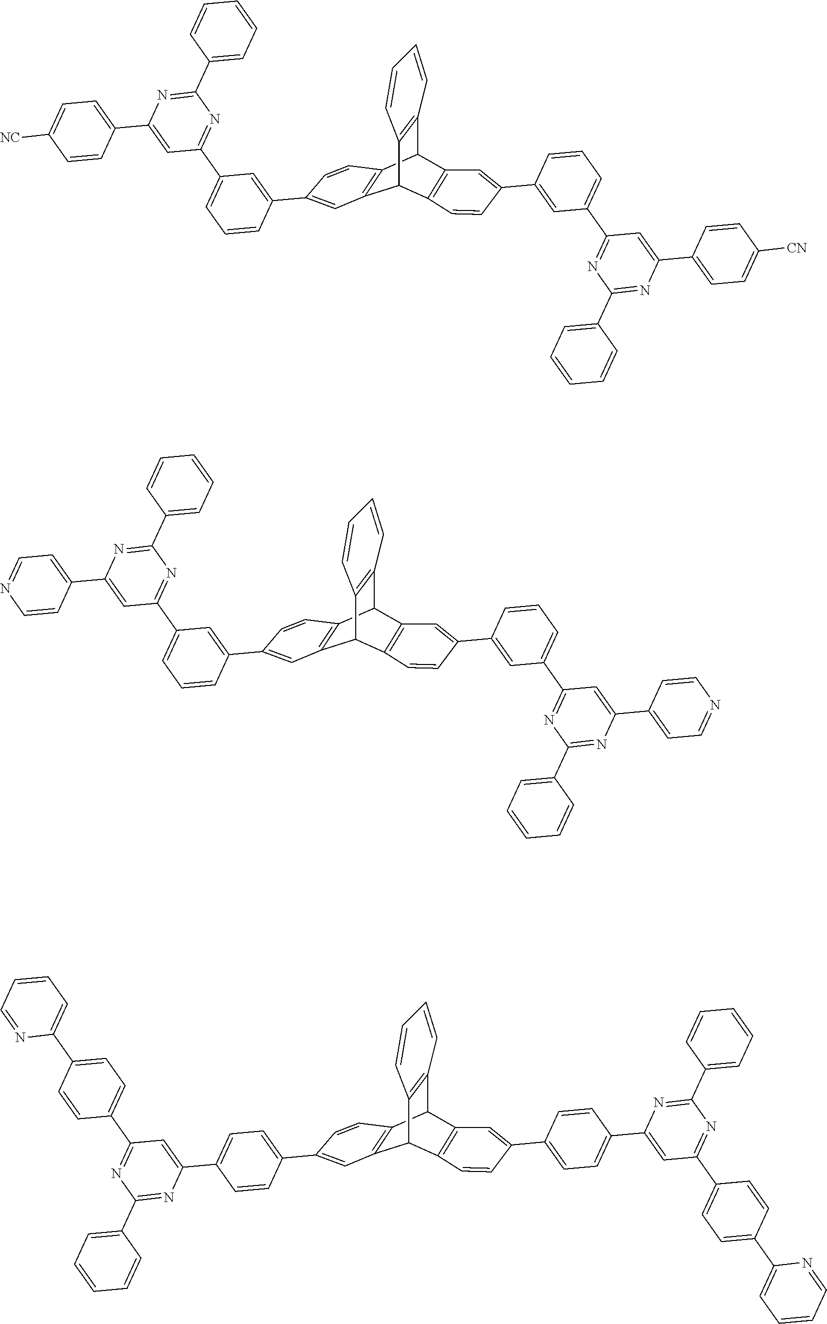 [SMILES: N#Cc1ccc(-c2cc(-c3cccc(-c4ccc5c(c4)C4c6ccccc6C5c5cc(-c6cccc(-c7cc(-c8ccc(C#N)cc8)nc(-c8ccccc8)n7)c6)ccc54)c3)nc(-c3ccccc3)n2)cc1 ; c1ccc(-c2nc(-c3ccncc3)cc(-c3cccc(-c4ccc5c(c4)C4c6ccccc6C5c5cc(-c6cccc(-c7cc(-c8ccncc8)nc(-c8ccccc8)n7)c6)ccc54)c3)n2)cc1 ; c1ccc(-c2nc(-c3ccc(-c4ccc5c(c4)C4c6ccccc6C5c5cc(-c6ccc(-c7cc(-c8ccc(-c9ccccn9)cc8)nc(-c8ccccc8)n7)cc6)ccc54)cc3)cc(-c3ccc(-c4ccccn4)cc3)n2)cc1]

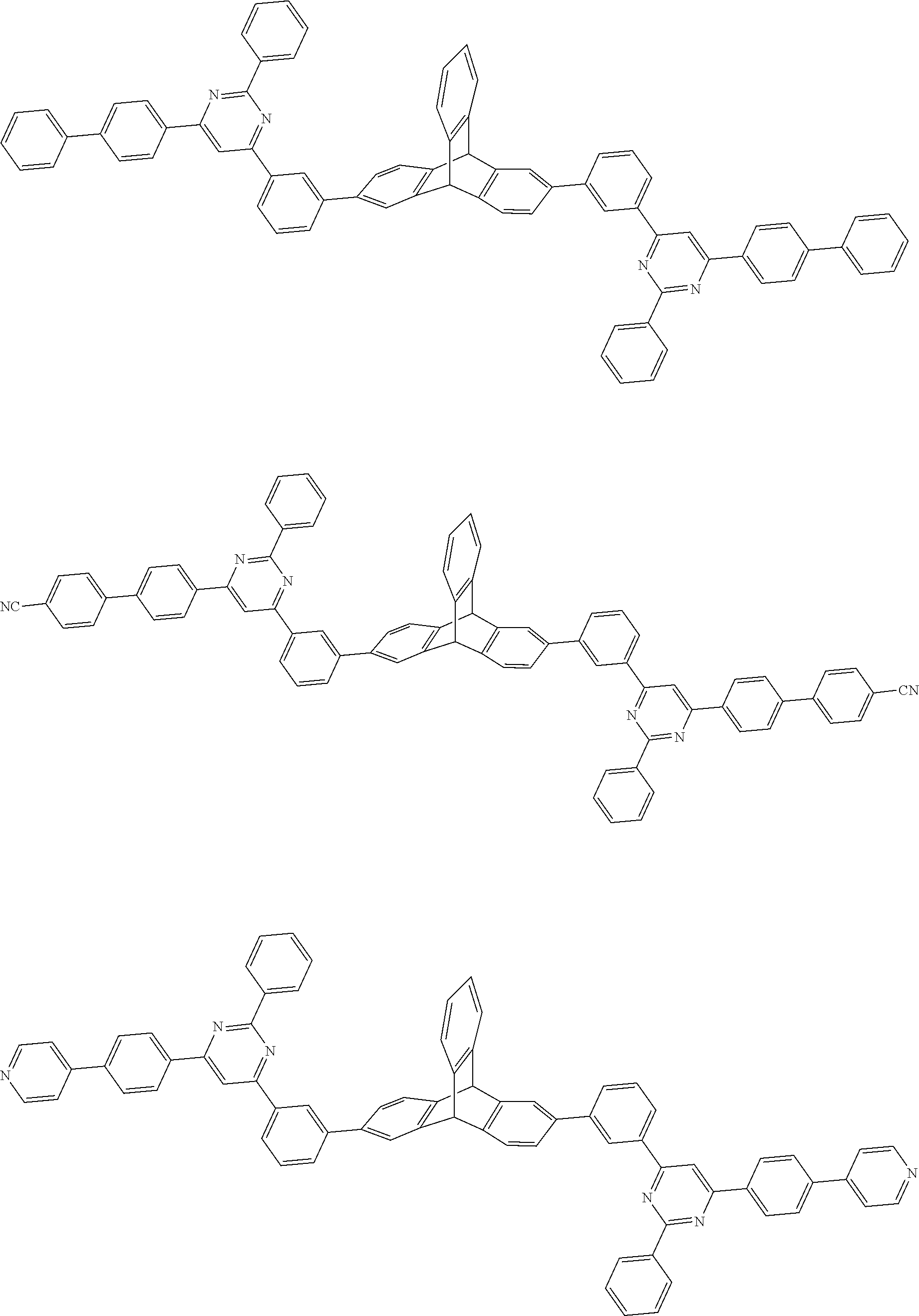 [SMILES: c1ccc(-c2ccc(-c3cc(-c4cccc(-c5ccc6c(c5)C5c7ccccc7C6c6cc(-c7cccc(-c8cc(-c9ccc(-c%10ccccc%10)cc9)nc(-c9ccccc9)n8)c7)ccc65)c4)nc(-c4ccccc4)n3)cc2)cc1 ; N#Cc1ccc(-c2ccc(-c3cc(-c4cccc(-c5ccc6c(c5)C5c7ccccc7C6c6cc(-c7cccc(-c8cc(-c9ccc(-c%10ccc(C#N)cc%10)cc9)nc(-c9ccccc9)n8)c7)ccc65)c4)nc(-c4ccccc4)n3)cc2)cc1 ; c1ccc(-c2nc(-c3ccc(-c4ccncc4)cc3)cc(-c3cccc(-c4ccc5c(c4)C4c6ccccc6C5c5cc(-c6cccc(-c7cc(-c8ccc(-c9ccncc9)cc8)nc(-c8ccccc8)n7)c6)ccc54)c3)n2)cc1]

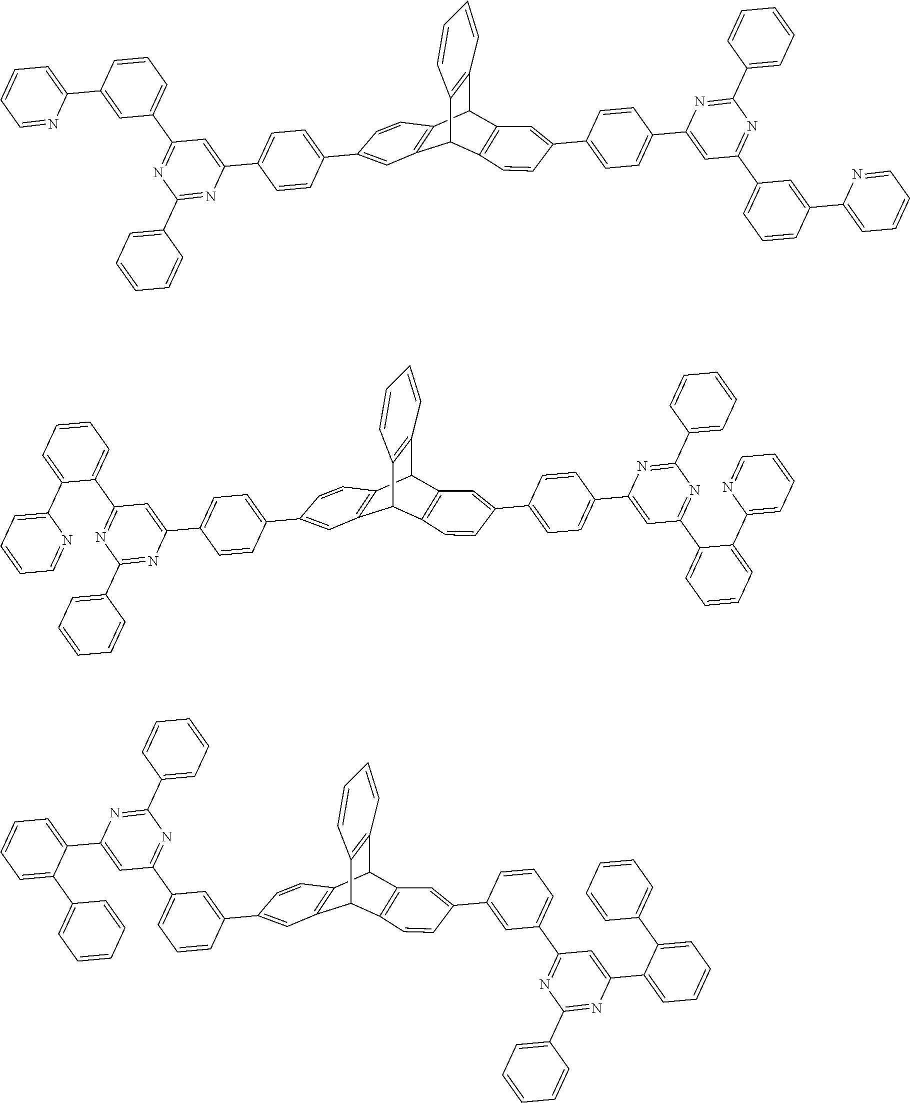 [SMILES: c1ccc(-c2nc(-c3ccc(-c4ccc5c(c4)C4c6ccccc6C5c5cc(-c6ccc(-c7cc(-c8cccc(-c9ccccn9)c8)nc(-c8ccccc8)n7)cc6)ccc54)cc3)cc(-c3cccc(-c4ccccn4)c3)n2)cc1 ; c1ccc(-c2nc(-c3ccc(-c4ccc5c(c4)C4c6ccccc6C5c5cc(-c6ccc(-c7cc(-c8ccccc8-c8ccccn8)nc(-c8ccccc8)n7)cc6)ccc54)cc3)cc(-c3ccccc3-c3ccccn3)n2)cc1 ; c1ccc(-c2nc(-c3cccc(-c4ccc5c(c4)C4c6ccccc6C5c5cc(-c6cccc(-c7cc(-c8ccccc8-c8ccccc8)nc(-c8ccccc8)n7)c6)ccc54)c3)cc(-c3ccccc3-c3ccccc3)n2)cc1]

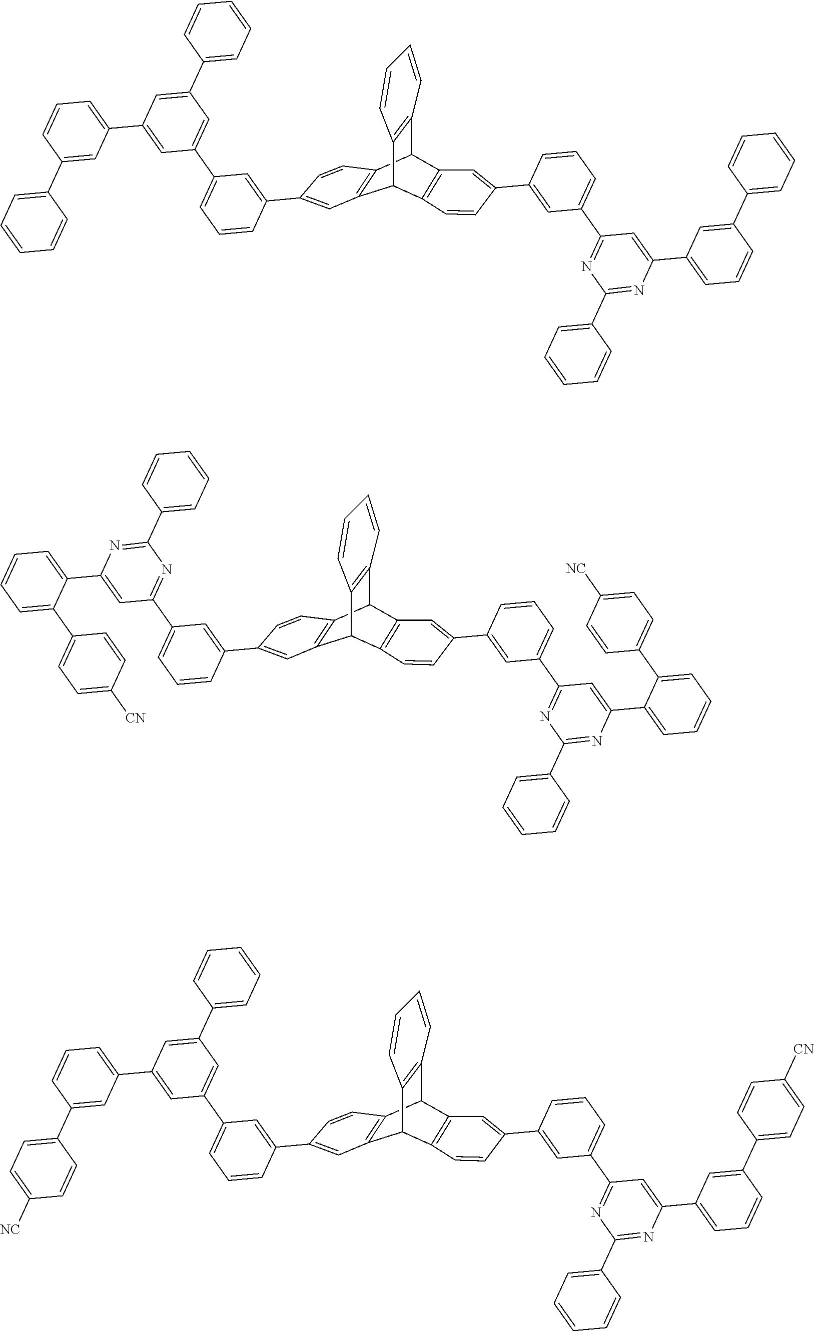 [SMILES: c1ccc(-c2cccc(-c3cc(-c4ccccc4)cc(-c4cccc(-c5ccc6c(c5)C5c7ccccc7C6c6cc(-c7cccc(-c8cc(-c9cccc(-c%10ccccc%10)c9)nc(-c9ccccc9)n8)c7)ccc65)c4)c3)c2)cc1 ; N#Cc1ccc(-c2ccccc2-c2cc(-c3cccc(-c4ccc5c(c4)C4c6ccccc6C5c5cc(-c6cccc(-c7cc(-c8ccccc8-c8ccc(C#N)cc8)nc(-c8ccccc8)n7)c6)ccc54)c3)nc(-c3ccccc3)n2)cc1 ; N#Cc1ccc(-c2cccc(-c3cc(-c4ccccc4)cc(-c4cccc(-c5ccc6c(c5)C5c7ccccc7C6c6cc(-c7cccc(-c8cc(-c9cccc(-c%10ccc(C#N)cc%10)c9)nc(-c9ccccc9)n8)c7)ccc65)c4)c3)c2)cc1]

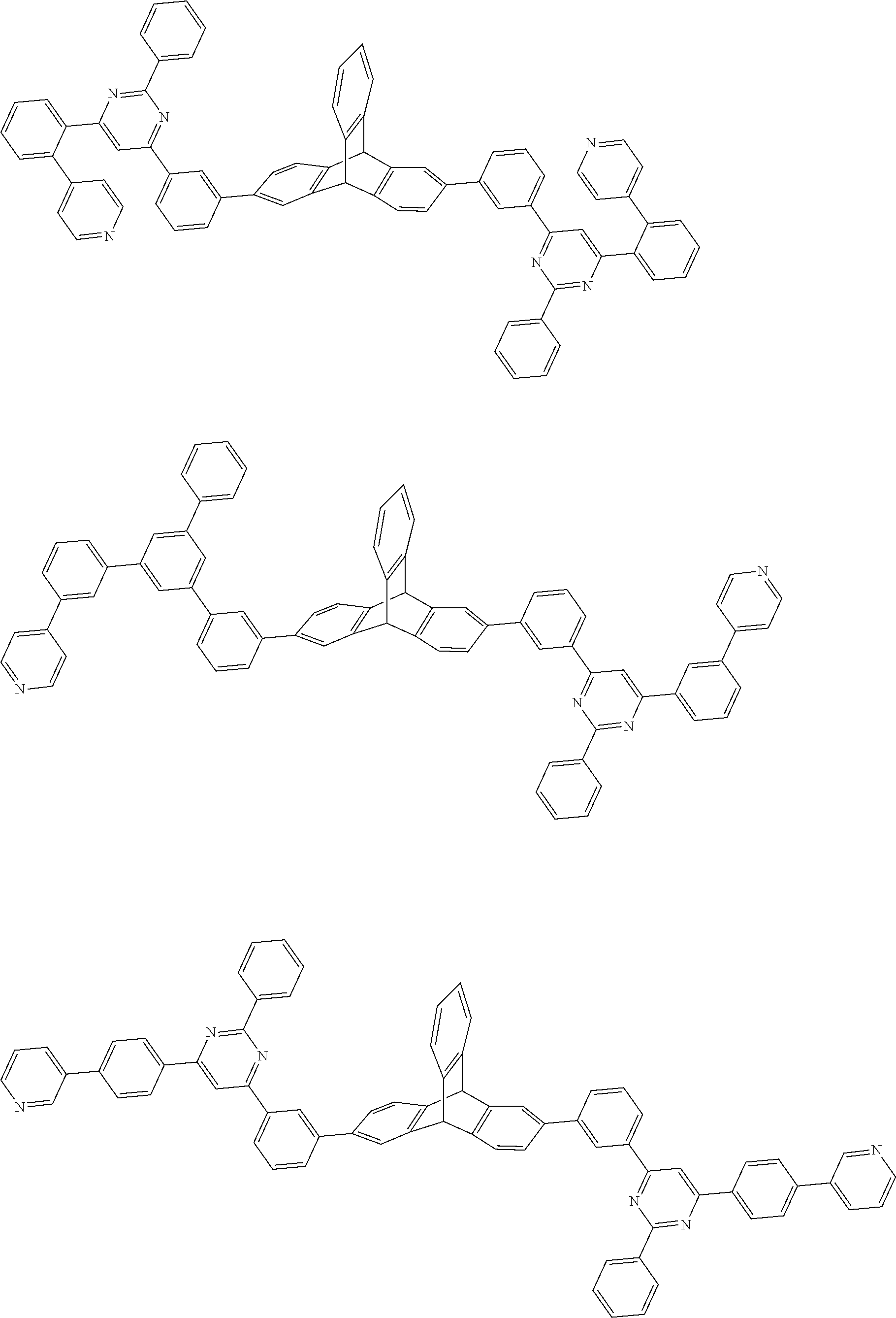 [SMILES: c1ccc(-c2nc(-c3cccc(-c4ccc5c(c4)C4c6ccccc6C5c5cc(-c6cccc(-c7cc(-c8ccccc8-c8ccncc8)nc(-c8ccccc8)n7)c6)ccc54)c3)cc(-c3ccccc3-c3ccncc3)n2)cc1 ; c1ccc(-c2cc(-c3cccc(-c4ccncc4)c3)cc(-c3cccc(-c4ccc5c(c4)C4c6ccccc6C5c5cc(-c6cccc(-c7cc(-c8cccc(-c9ccncc9)c8)nc(-c8ccccc8)n7)c6)ccc54)c3)c2)cc1 ; c1ccc(-c2nc(-c3ccc(-c4cccnc4)cc3)cc(-c3cccc(-c4ccc5c(c4)C4c6ccccc6C5c5cc(-c6cccc(-c7cc(-c8ccc(-c9cccnc9)cc8)nc(-c8ccccc8)n7)c6)ccc54)c3)n2)cc1]

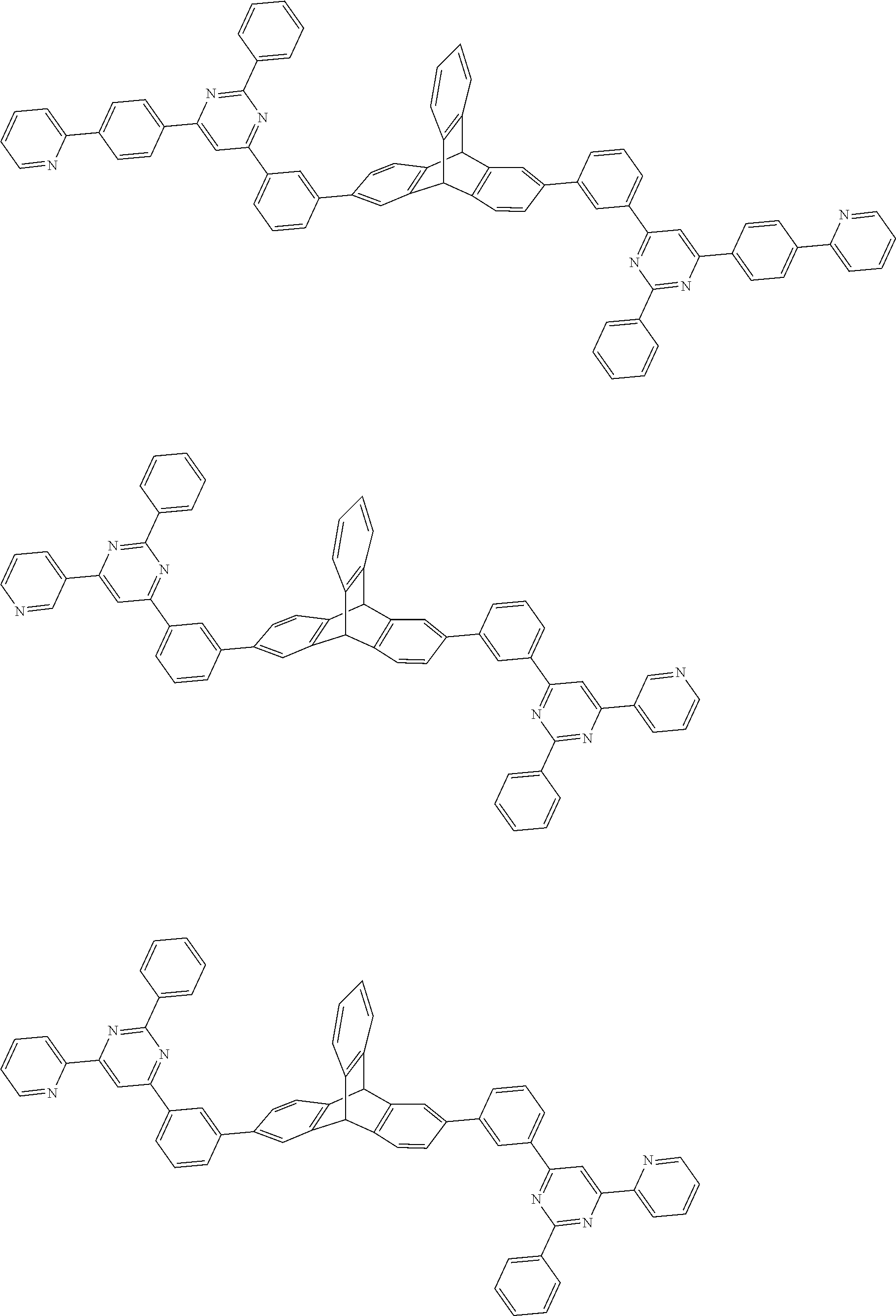 [SMILES: c1ccc(-c2nc(-c3ccc(-c4ccccn4)cc3)cc(-c3cccc(-c4ccc5c(c4)C4c6ccccc6C5c5cc(-c6cccc(-c7cc(-c8ccc(-c9ccccn9)cc8)nc(-c8ccccc8)n7)c6)ccc54)c3)n2)cc1 ; c1ccc(-c2nc(-c3cccnc3)cc(-c3cccc(-c4ccc5c(c4)C4c6ccccc6C5c5cc(-c6cccc(-c7cc(-c8cccnc8)nc(-c8ccccc8)n7)c6)ccc54)c3)n2)cc1 ; c1ccc(-c2nc(-c3cccc(-c4ccc5c(c4)C4c6ccccc6C5c5cc(-c6cccc(-c7cc(-c8ccccn8)nc(-c8ccccc8)n7)c6)ccc54)c3)cc(-c3ccccn3)n2)cc1]

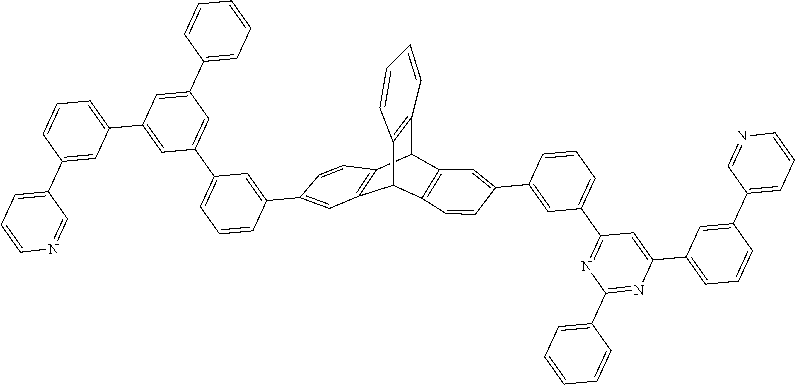 [SMILES: c1ccc(-c2cc(-c3cccc(-c4cccnc4)c3)cc(-c3cccc(-c4ccc5c(c4)C4c6ccccc6C5c5cc(-c6cccc(-c7cc(-c8cccc(-c9cccnc9)c8)nc(-c8ccccc8)n7)c6)ccc54)c3)c2)cc1]

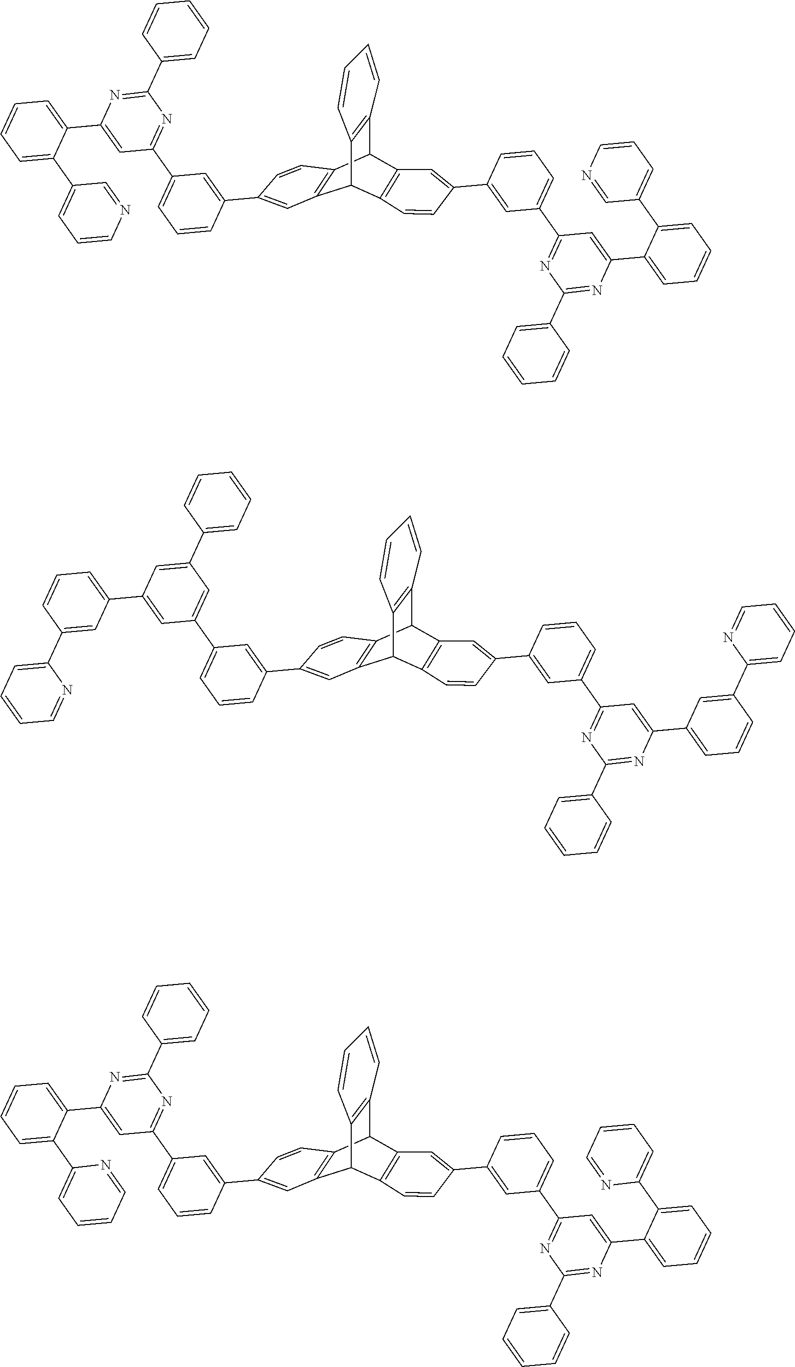 [SMILES: c1ccc(-c2nc(-c3cccc(-c4ccc5c(c4)C4c6ccccc6C5c5cc(-c6cccc(-c7cc(-c8ccccc8-c8cccnc8)nc(-c8ccccc8)n7)c6)ccc54)c3)cc(-c3ccccc3-c3cccnc3)n2)cc1 ; c1ccc(-c2cc(-c3cccc(-c4ccc5c(c4)C4c6ccccc6C5c5cc(-c6cccc(-c7cc(-c8cccc(-c9ccccn9)c8)nc(-c8ccccc8)n7)c6)ccc54)c3)cc(-c3cccc(-c4ccccn4)c3)c2)cc1 ; c1ccc(-c2nc(-c3cccc(-c4ccc5c(c4)C4c6ccccc6C5c5cc(-c6cccc(-c7cc(-c8ccccc8-c8ccccn8)nc(-c8ccccc8)n7)c6)ccc54)c3)cc(-c3ccccc3-c3ccccn3)n2)cc1]

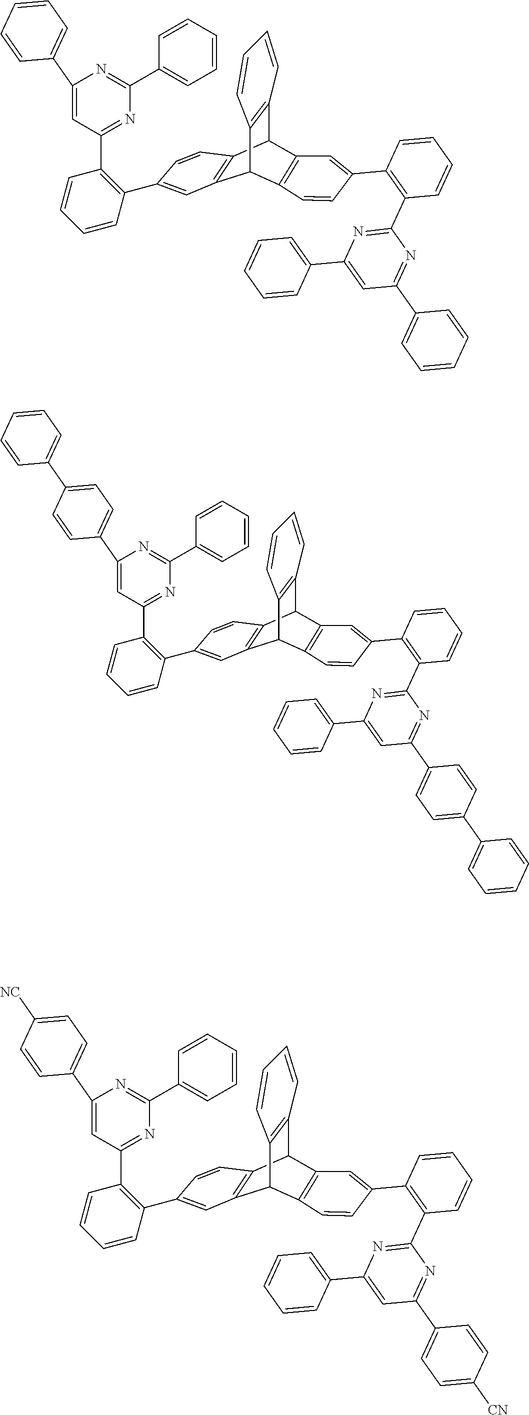 [SMILES: c1ccc(-c2cc(-c3ccccc3-c3ccc4c(c3)C3c5ccccc5C4c4cc(-c5ccccc5-c5nc(-c6ccccc6)cc(-c6ccccc6)n5)ccc43)nc(-c3ccccc3)n2)cc1 ; c1ccc(-c2ccc(-c3cc(-c4ccccc4-c4ccc5c(c4)C4c6ccccc6C5c5cc(-c6ccccc6-c6nc(-c7ccccc7)cc(-c7ccc(-c8ccccc8)cc7)n6)ccc54)nc(-c4ccccc4)n3)cc2)cc1 ; N#Cc1ccc(-c2cc(-c3ccccc3-c3ccc4c(c3)C3c5ccccc5C4c4cc(-c5ccccc5-c5nc(-c6ccccc6)cc(-c6ccc(C#N)cc6)n5)ccc43)nc(-c3ccccc3)n2)cc1]

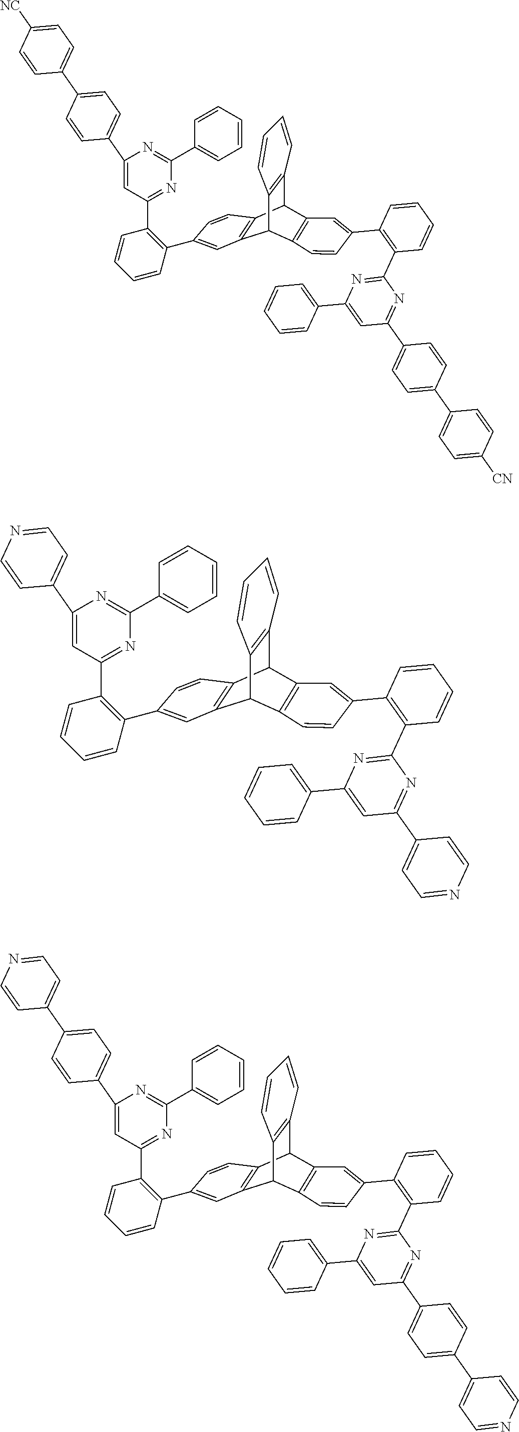 [SMILES: N#Cc1ccc(-c2ccc(-c3cc(-c4ccccc4-c4ccc5c(c4)C4c6ccccc6C5c5cc(-c6ccccc6-c6nc(-c7ccccc7)cc(-c7ccc(-c8ccc(C#N)cc8)cc7)n6)ccc54)nc(-c4ccccc4)n3)cc2)cc1 ; c1ccc(-c2cc(-c3ccncc3)nc(-c3ccccc3-c3ccc4c(c3)C3c5ccccc5C4c4cc(-c5ccccc5-c5cc(-c6ccncc6)nc(-c6ccccc6)n5)ccc43)n2)cc1 ; c1ccc(-c2cc(-c3ccc(-c4ccncc4)cc3)nc(-c3ccccc3-c3ccc4c(c3)C3c5ccccc5C4c4cc(-c5ccccc5-c5cc(-c6ccc(-c7ccncc7)cc6)nc(-c6ccccc6)n5)ccc43)n2)cc1]

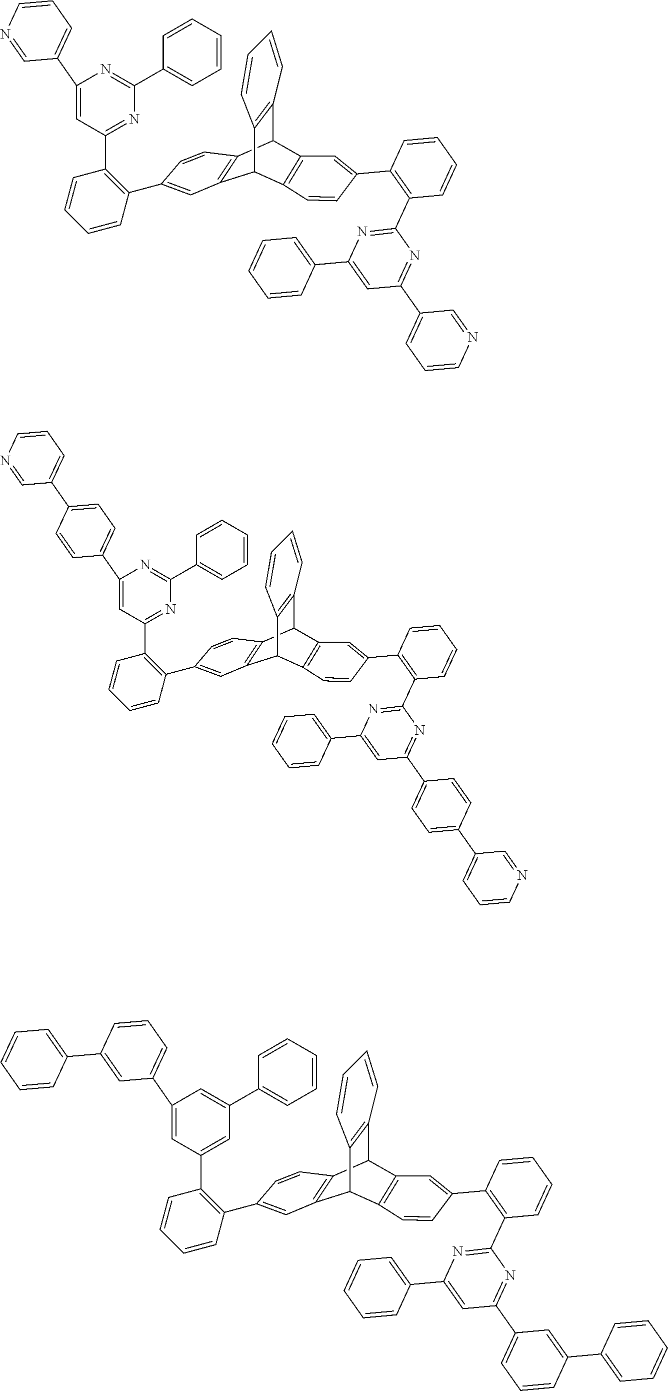 [SMILES: c1ccc(-c2cc(-c3cccnc3)nc(-c3ccccc3-c3ccc4c(c3)C3c5ccccc5C4c4cc(-c5ccccc5-c5cc(-c6cccnc6)nc(-c6ccccc6)n5)ccc43)n2)cc1 ; c1ccc(-c2cc(-c3ccc(-c4cccnc4)cc3)nc(-c3ccccc3-c3ccc4c(c3)C3c5ccccc5C4c4cc(-c5ccccc5-c5cc(-c6ccc(-c7cccnc7)cc6)nc(-c6ccccc6)n5)ccc43)n2)cc1 ; c1ccc(-c2cccc(-c3cc(-c4ccccc4)cc(-c4ccccc4-c4ccc5c(c4)C4c6ccccc6C5c5cc(-c6ccccc6-c6nc(-c7ccccc7)cc(-c7cccc(-c8ccccc8)c7)n6)ccc54)c3)c2)cc1]

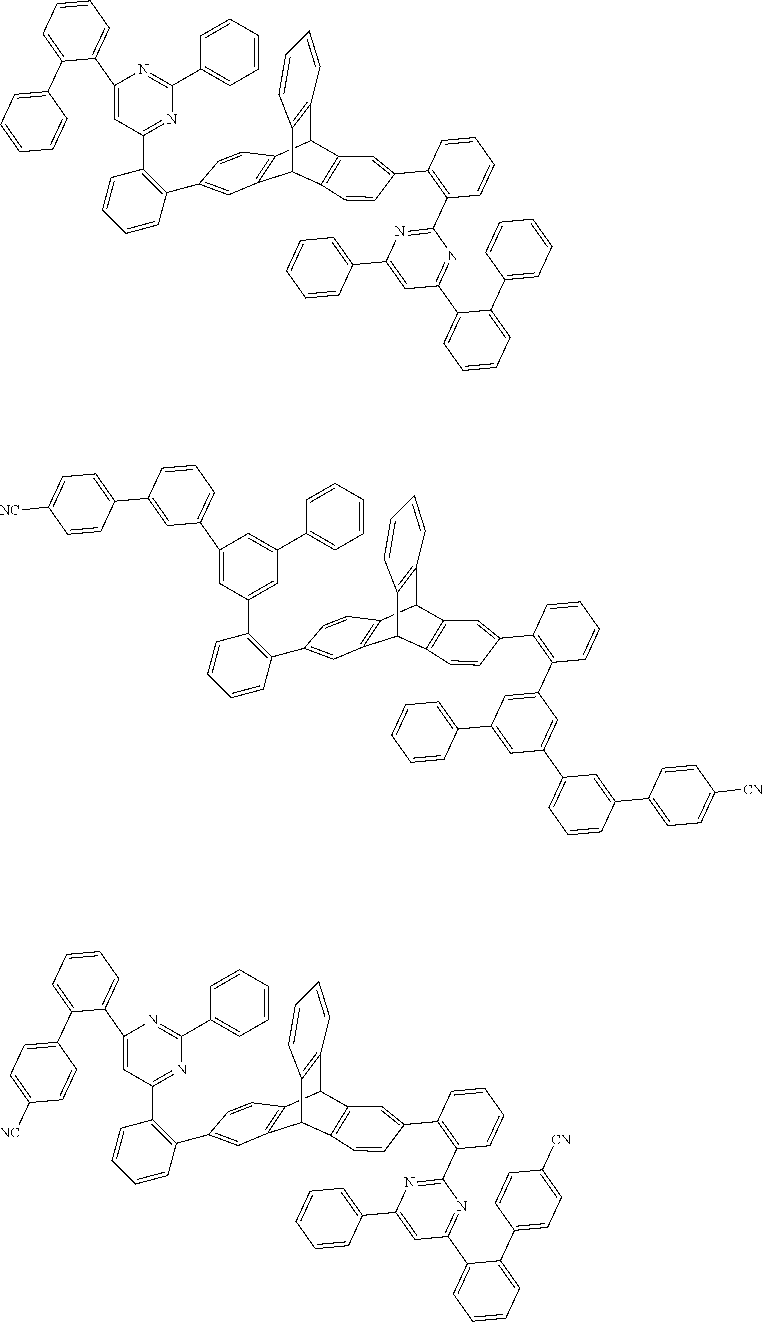 [SMILES: c1ccc(-c2cc(-c3ccccc3-c3ccccc3)nc(-c3ccccc3-c3ccc4c(c3)C3c5ccccc5C4c4cc(-c5ccccc5-c5cc(-c6ccccc6-c6ccccc6)nc(-c6ccccc6)n5)ccc43)n2)cc1 ; N#Cc1ccc(-c2cccc(-c3cc(-c4ccccc4)cc(-c4ccccc4-c4ccc5c(c4)C4c6ccccc6C5c5cc(-c6ccccc6-c6cc(-c7ccccc7)cc(-c7cccc(-c8ccc(C#N)cc8)c7)c6)ccc54)c3)c2)cc1 ; N#Cc1ccc(-c2ccccc2-c2cc(-c3ccccc3-c3ccc4c(c3)C3c5ccccc5C4c4cc(-c5ccccc5-c5nc(-c6ccccc6)cc(-c6ccccc6-c6ccc(C#N)cc6)n5)ccc43)nc(-c3ccccc3)n2)cc1]

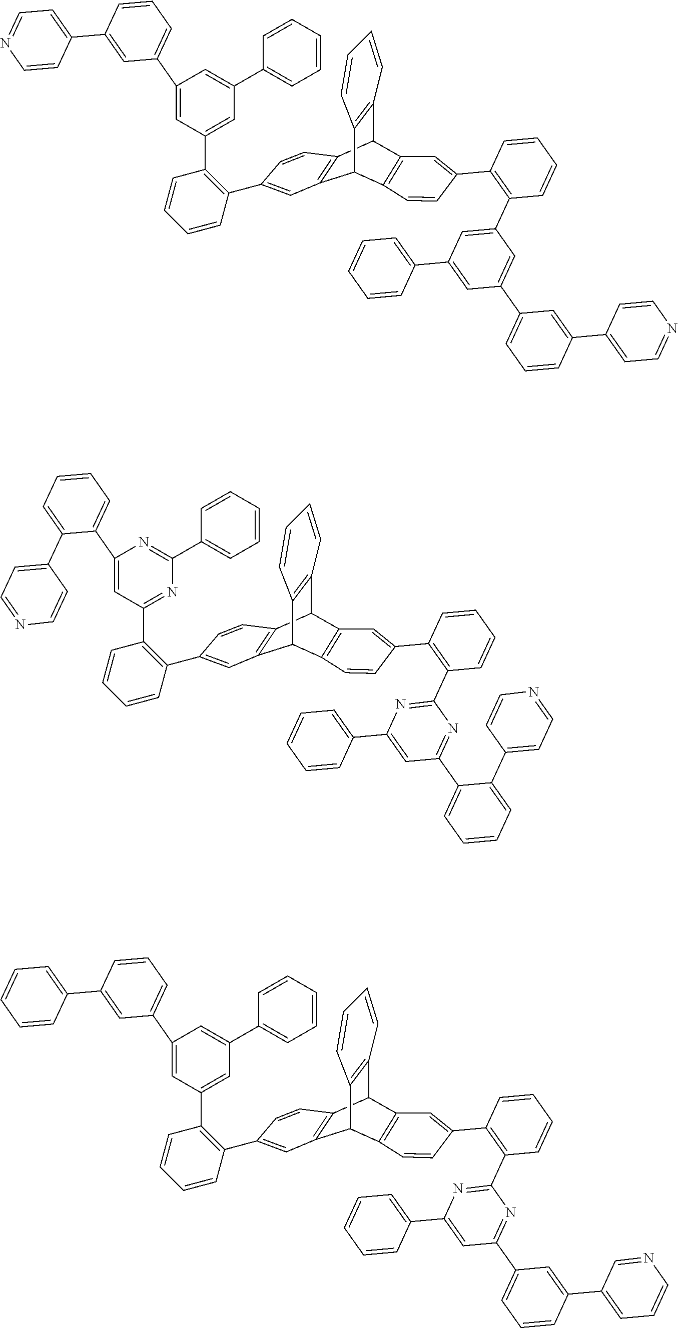 [SMILES: c1ccc(-c2cc(-c3cccc(-c4ccncc4)c3)cc(-c3ccccc3-c3ccc4c(c3)C3c5ccccc5C4c4cc(-c5ccccc5-c5cc(-c6ccccc6)cc(-c6cccc(-c7ccncc7)c6)c5)ccc43)c2)cc1 ; c1ccc(-c2cc(-c3ccccc3-c3ccncc3)nc(-c3ccccc3-c3ccc4c(c3)C3c5ccccc5C4c4cc(-c5ccccc5-c5cc(-c6ccccc6-c6ccncc6)nc(-c6ccccc6)n5)ccc43)n2)cc1 ; c1ccc(-c2cccc(-c3cc(-c4ccccc4)cc(-c4ccccc4-c4ccc5c(c4)C4c6ccccc6C5c5cc(-c6ccccc6-c6nc(-c7ccccc7)cc(-c7cccc(-c8cccnc8)c7)n6)ccc54)c3)c2)cc1]

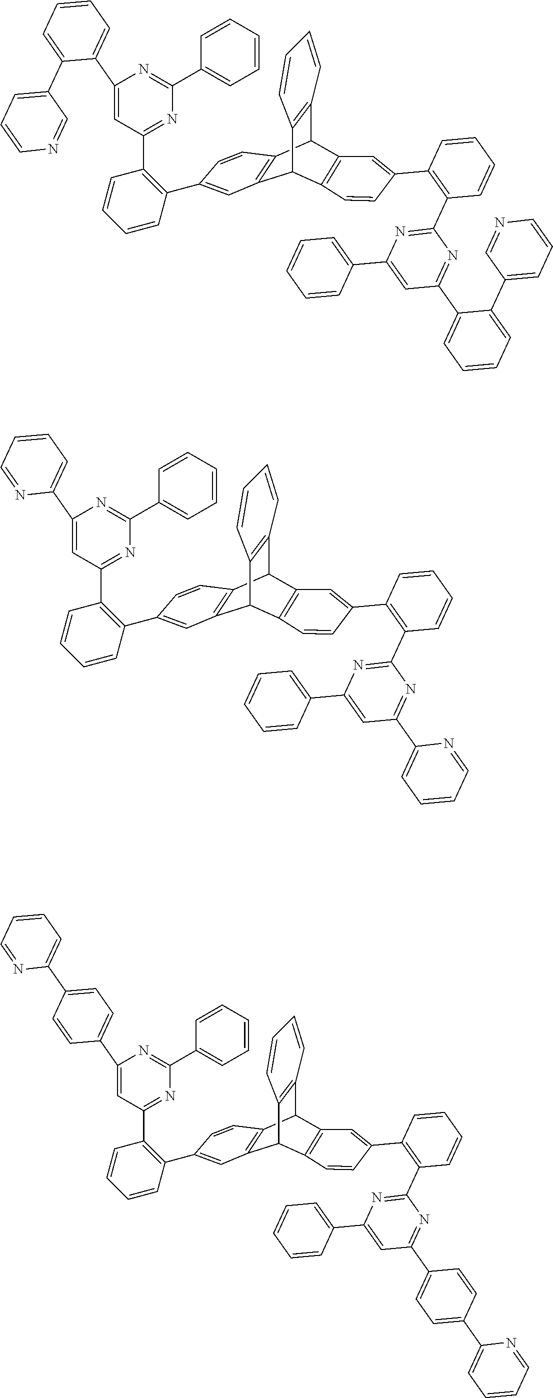 [SMILES: c1ccc(-c2cc(-c3ccccc3-c3cccnc3)nc(-c3ccccc3-c3ccc4c(c3)C3c5ccccc5C4c4cc(-c5ccccc5-c5cc(-c6ccccc6-c6cccnc6)nc(-c6ccccc6)n5)ccc43)n2)cc1 ; c1ccc(-c2cc(-c3ccccn3)nc(-c3ccccc3-c3ccc4c(c3)C3c5ccccc5C4c4cc(-c5ccccc5-c5cc(-c6ccccn6)nc(-c6ccccc6)n5)ccc43)n2)cc1 ; c1ccc(-c2cc(-c3ccc(-c4ccccn4)cc3)nc(-c3ccccc3-c3ccc4c(c3)C3c5ccccc5C4c4cc(-c5ccccc5-c5cc(-c6ccc(-c7ccccn7)cc6)nc(-c6ccccc6)n5)ccc43)n2)cc1]

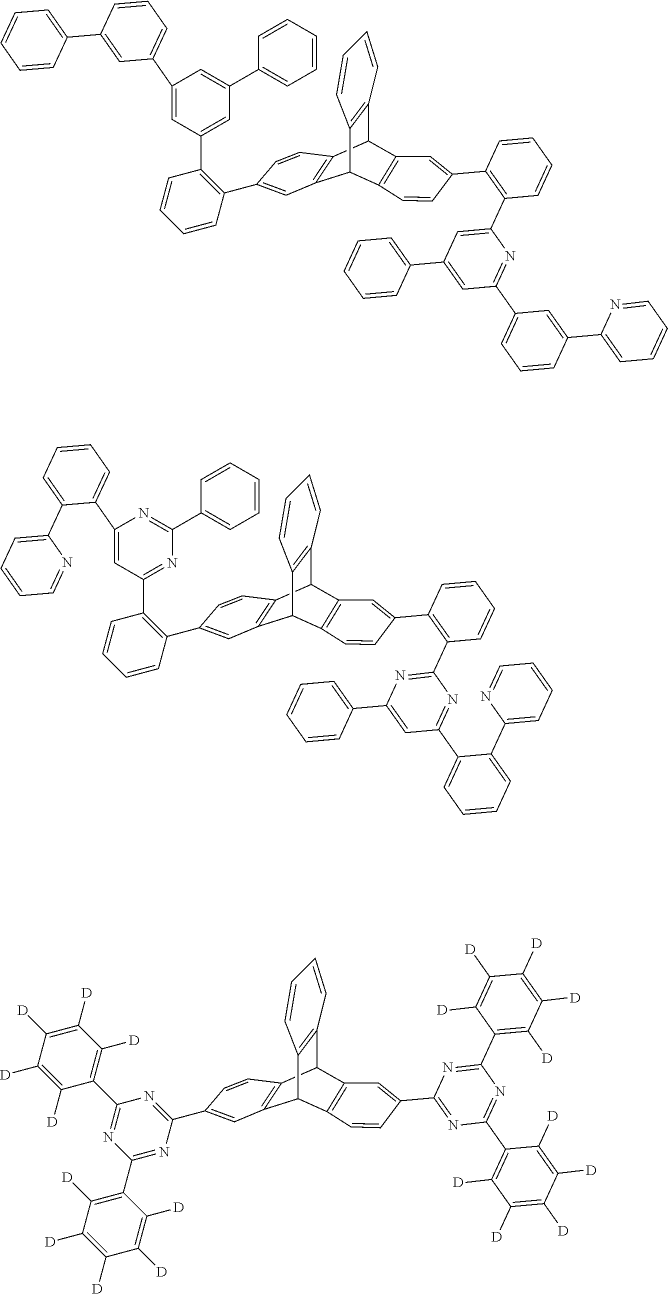 [SMILES: c1ccc(-c2cccc(-c3cc(-c4ccccc4)cc(-c4ccccc4-c4ccc5c(c4)C4c6ccccc6C5c5cc(-c6ccccc6-c6cc(-c7ccccc7)cc(-c7cccc(-c8ccccn8)c7)n6)ccc54)c3)c2)cc1 ; c1ccc(-c2cc(-c3ccccc3-c3ccccn3)nc(-c3ccccc3-c3ccc4c(c3)C3c5ccccc5C4c4cc(-c5ccccc5-c5cc(-c6ccccc6-c6ccccn6)nc(-c6ccccc6)n5)ccc43)n2)cc1 ; [2H]c1c([2H])c([2H])c(-c2nc(-c3ccc4c(c3)C3c5ccccc5C4c4cc(-c5nc(-c6c([2H])c([2H])c([2H])c([2H])c6[2H])nc(-c6c([2H])c([2H])c([2H])c([2H])c6[2H])n5)ccc43)nc(-c3c([2H])c([2H])c([2H])c([2H])c3[2H])n2)c([2H])c1[2H]]

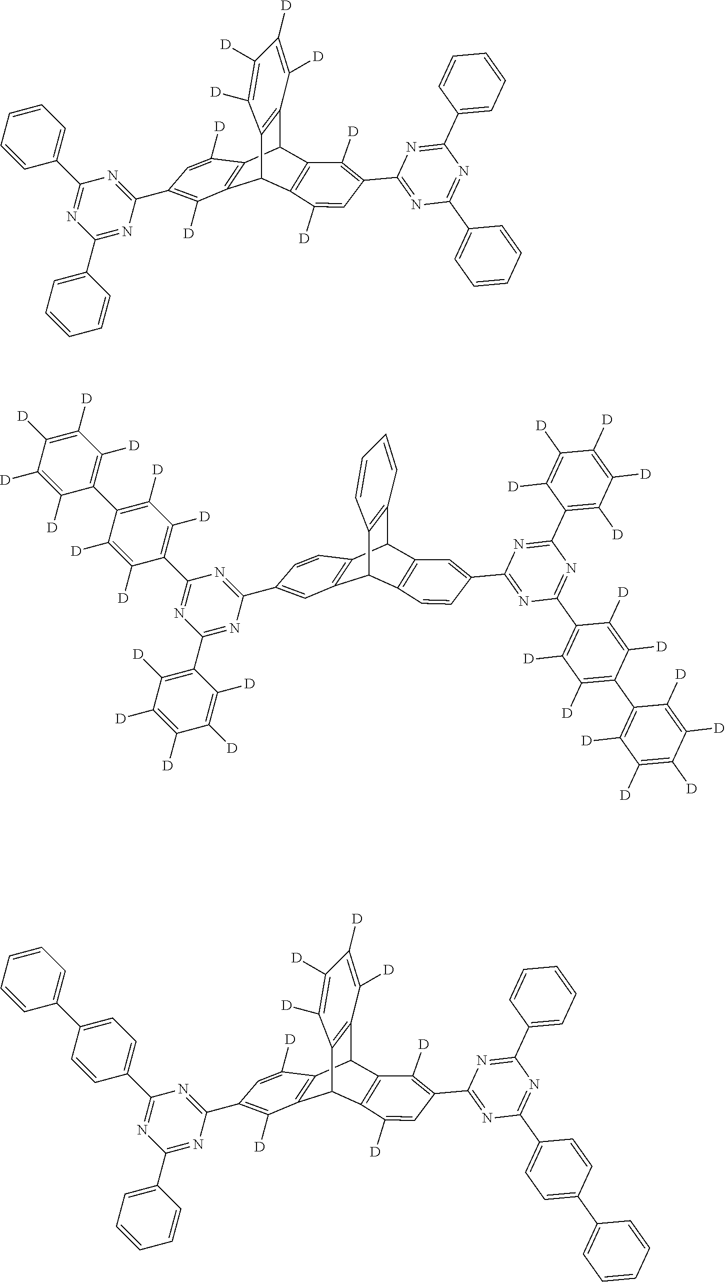

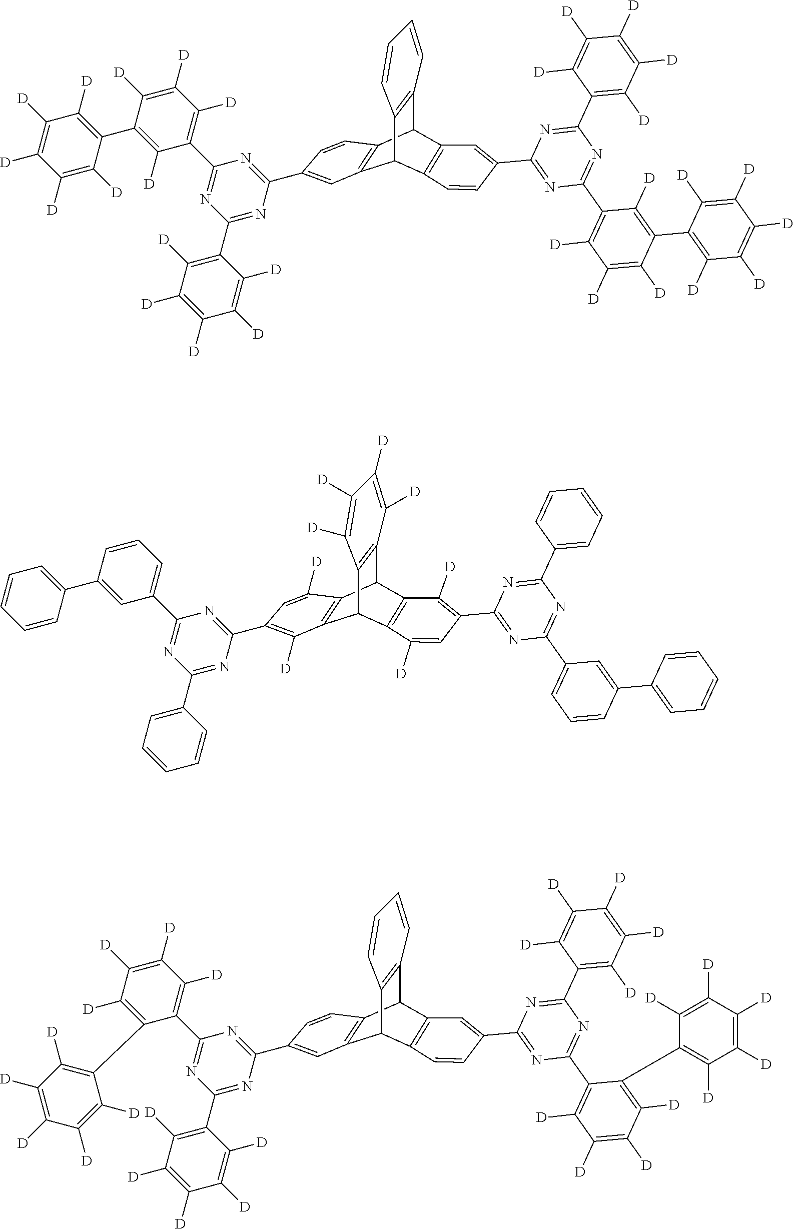

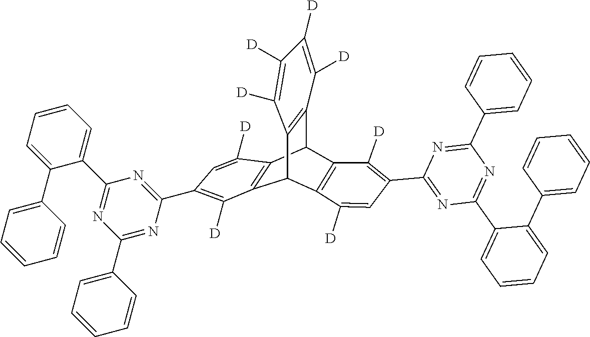 [SMILES: [2H]c1cc(-c2nc(-c3ccccc3)nc(-c3ccccc3-c3ccccc3)n2)c([2H])c2c1C1c3c([2H])c(-c4nc(-c5ccccc5)nc(-c5ccccc5-c5ccccc5)n4)cc([2H])c3C2c2c([2H])c([2H])c([2H])c([2H])c21]

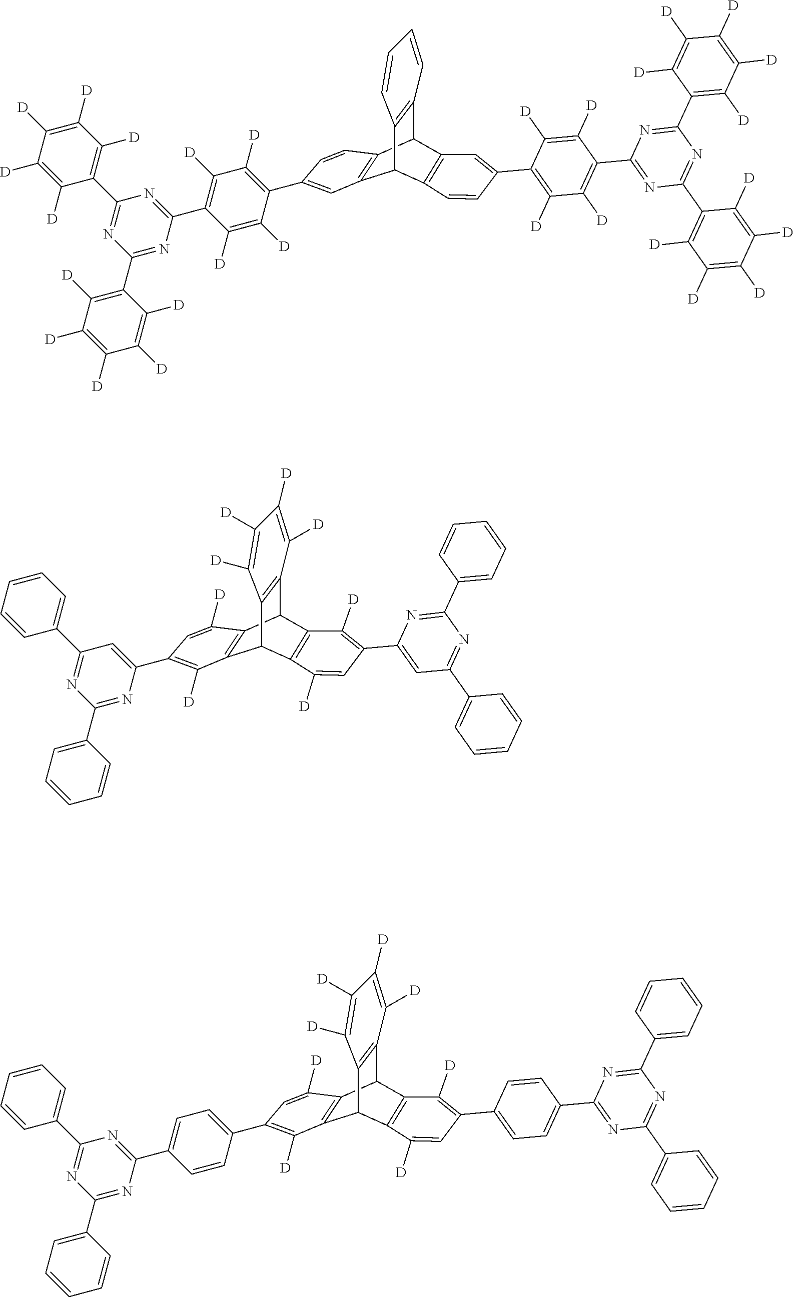

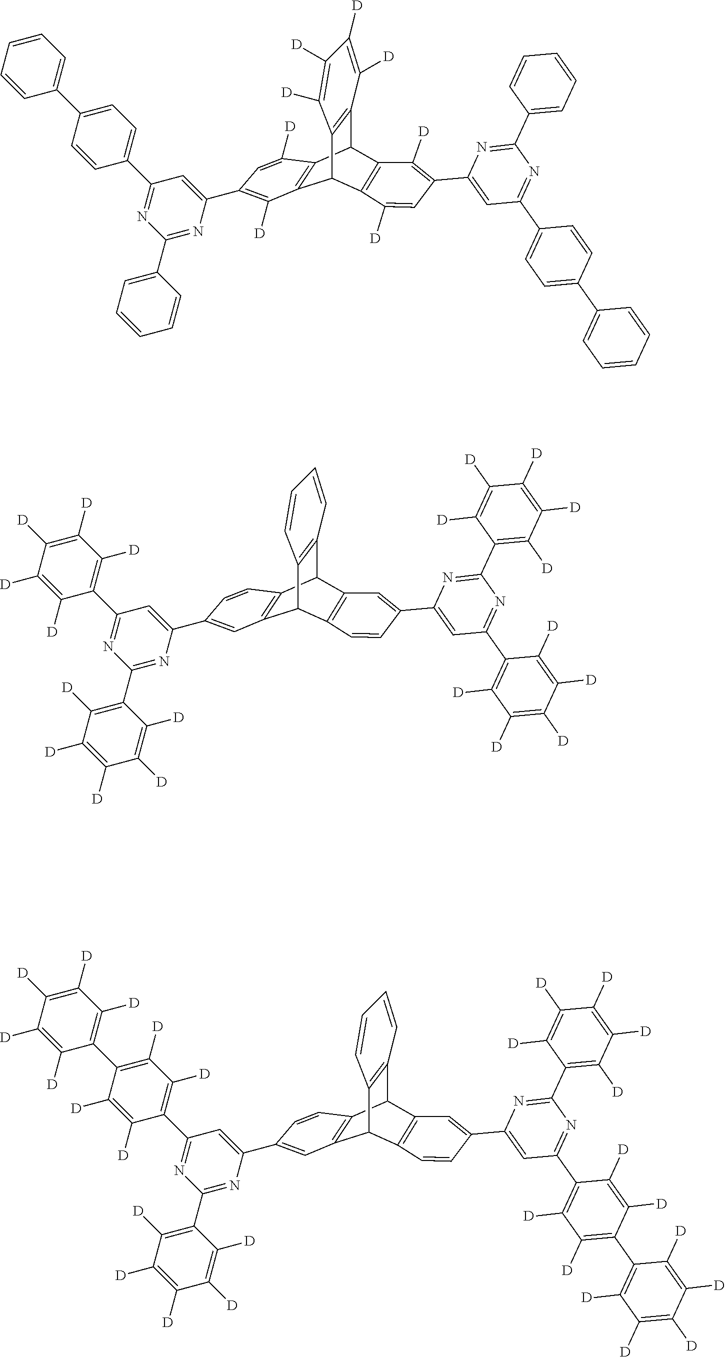

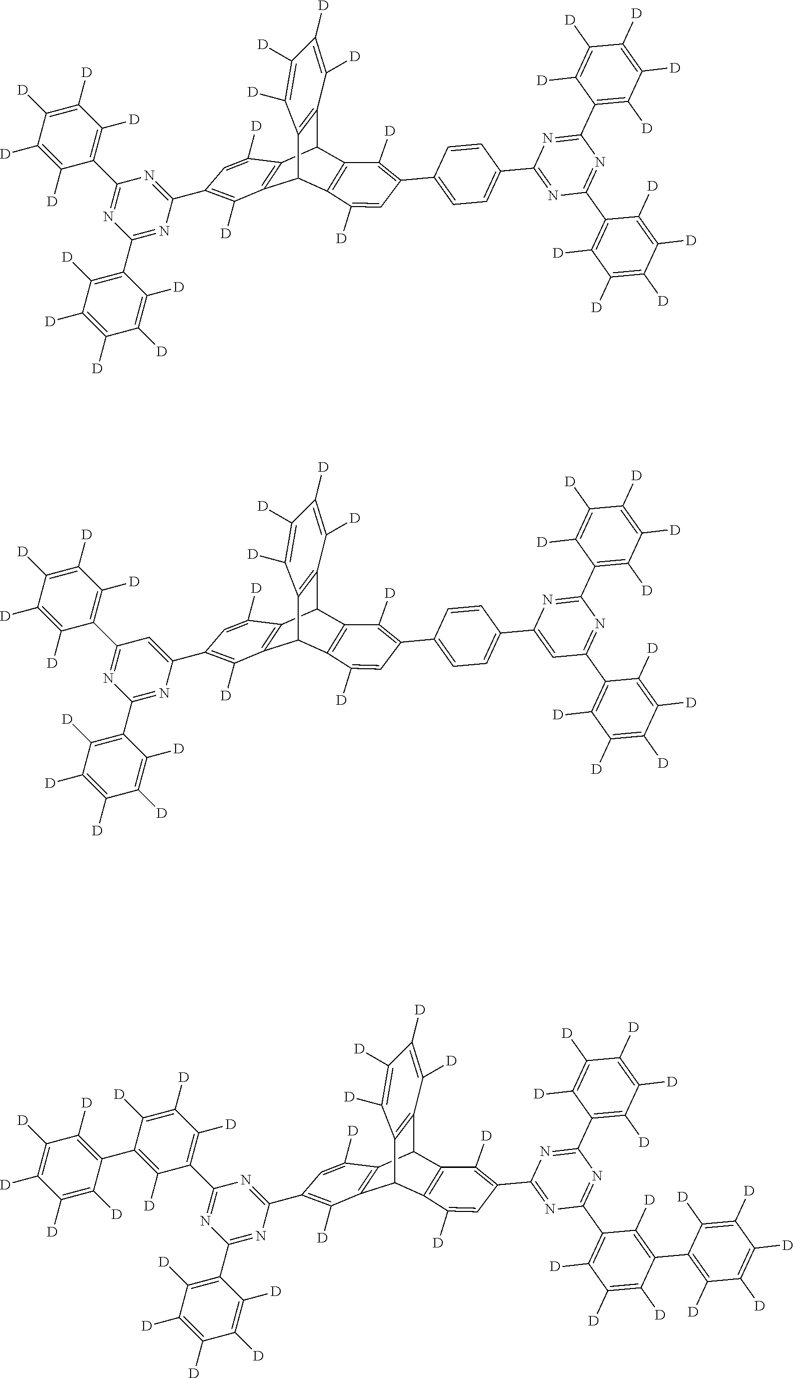

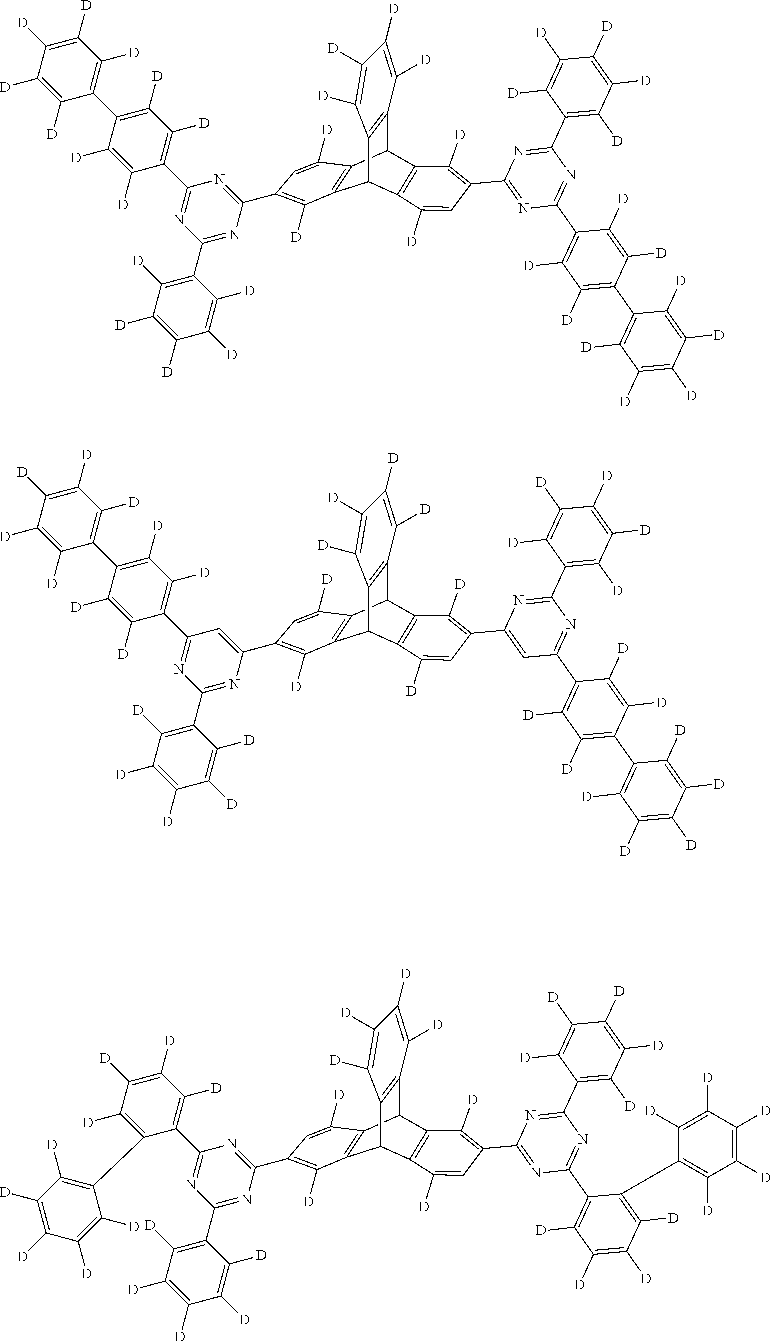

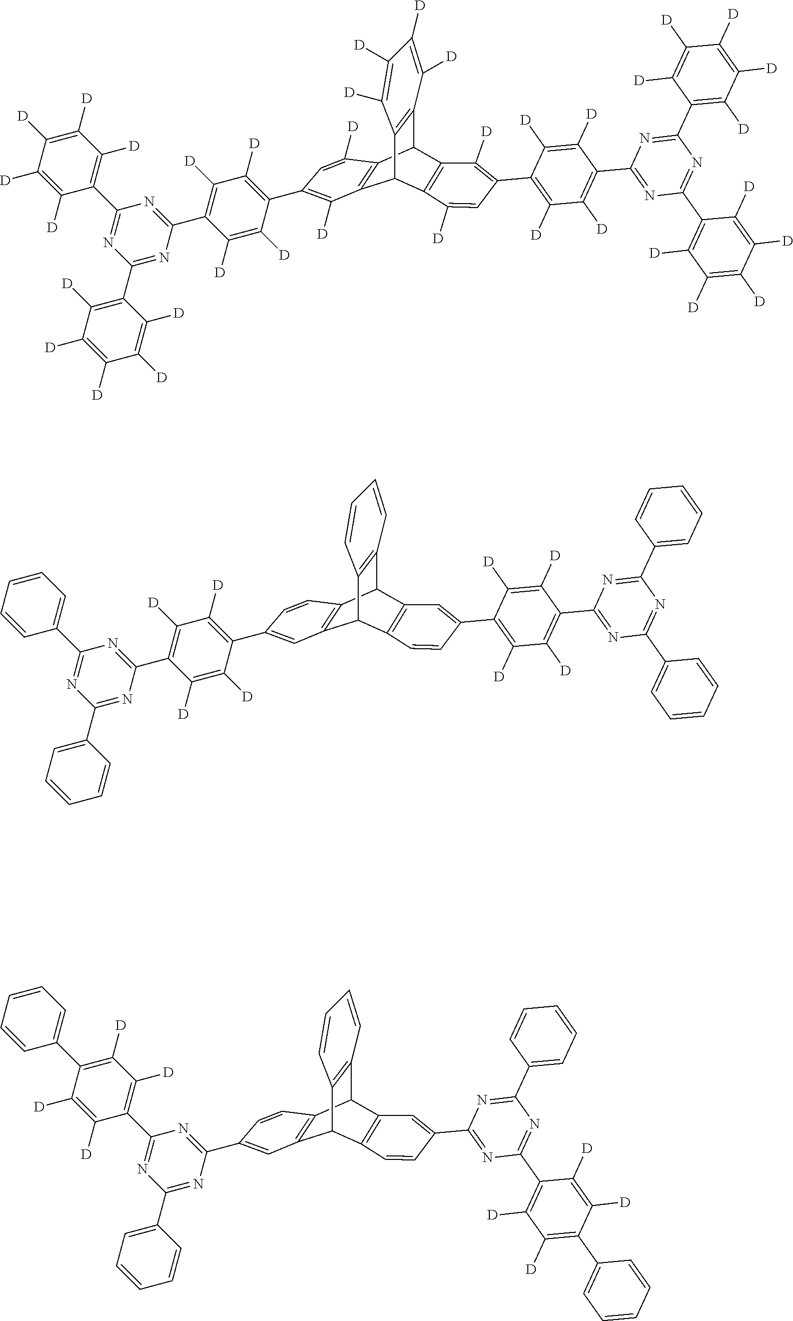

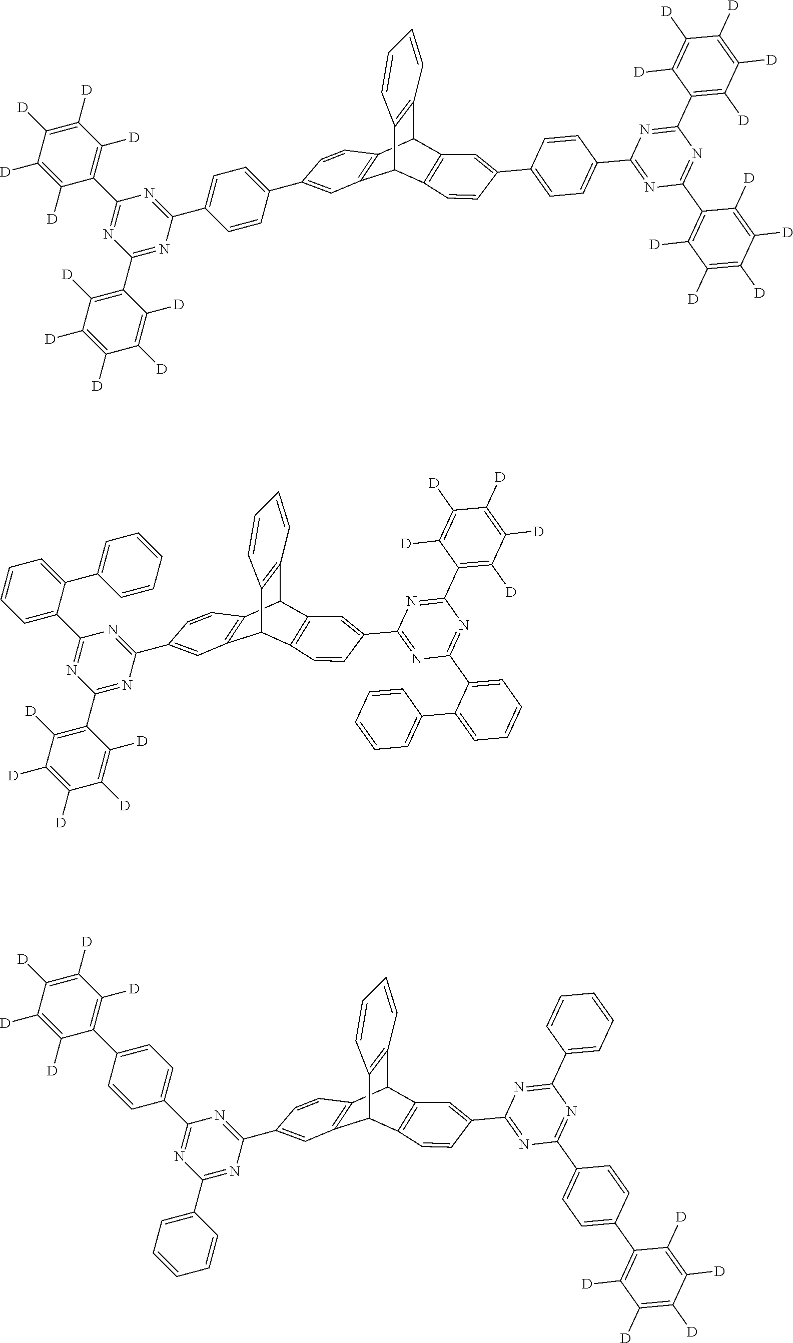

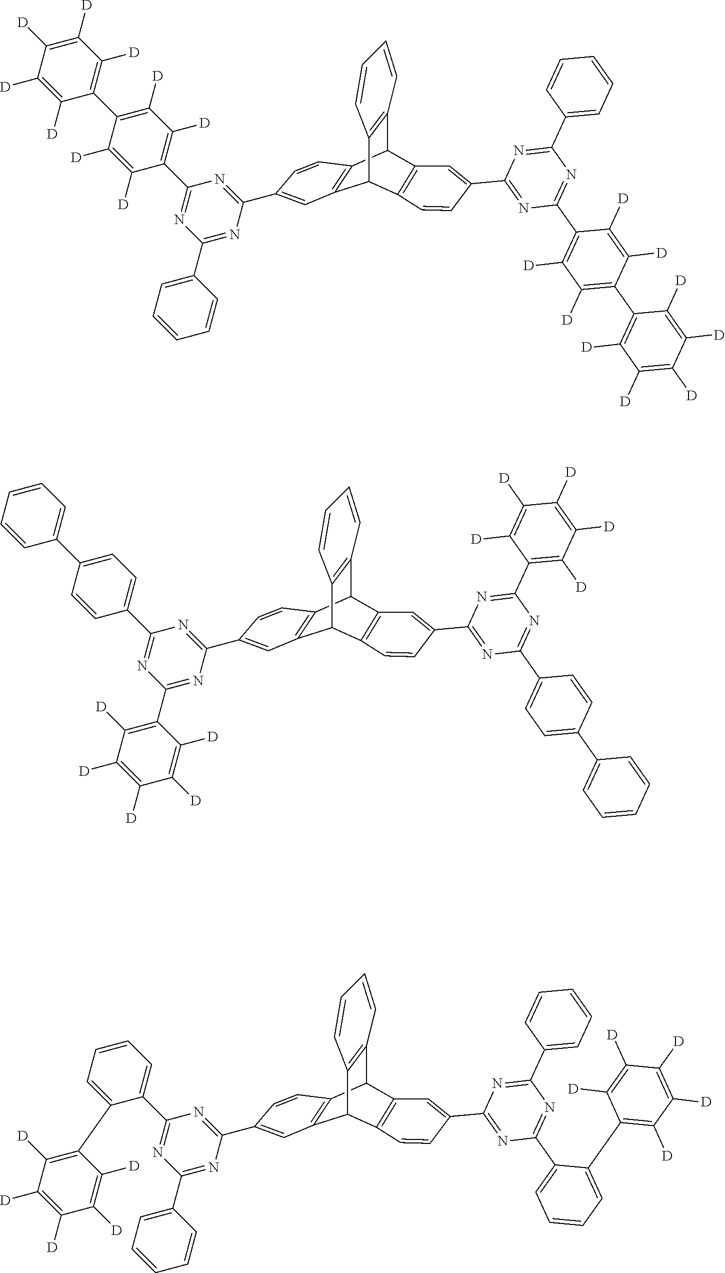

### Second Exemplary Embodiment

### Mixture

A mixture according to the second exemplary embodiment includes the compound according to the first exemplary embodiment as a first compound and a second compound that is an enantiomer of the first compound.

Examples of the combination of the compounds included in the mixture according to the second exemplary embodiment include:

a combination of the compound represented by the formula (10-1a) that serves as a first compound and the compound represented by the formula (10-1b) that serves as a second compound;

a combination of the compound represented by the formula (10-2a) that serves as a first compound and the compound represented by the formula (10-2b) that serves as a second compound; and

a combination of the compound represented by the formula (10-3a) that serves as a first compound and the compound represented by the formula (10-3b) that serves as a second compound.

The mixture according to the second exemplary embodiment can be suitably used as a material for an electron transporting zone (preferably, a material for an electron transporting layer) of an organic EL device.

The mixture according to the second exemplary embodiment may further include another compound in addition to the first compound (the compound according to the first exemplary embodiment) and the second compound that is an enantiomer of the first compound.

According to the second exemplary embodiment, a mixture capable of enhancing the performance of an organic EL device and, in particular, a mixture capable of reducing the drive voltage of an organic EL device can be provided.

Specific examples [1] to [10] of the combination of the first and second compounds included in the mixture according to the second exemplary embodiment are described below. Note that the mixture according to the invention is not limited to the specific examples below.

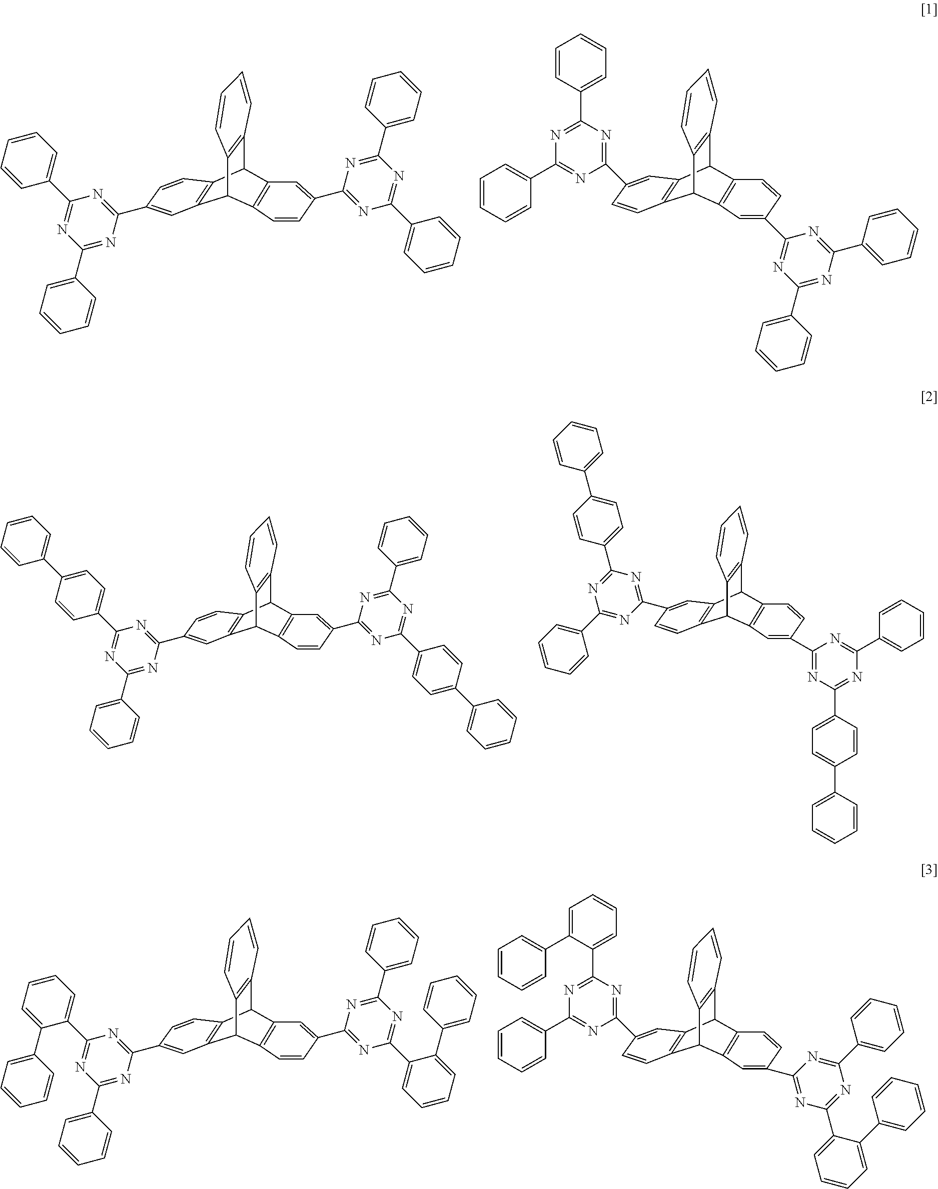

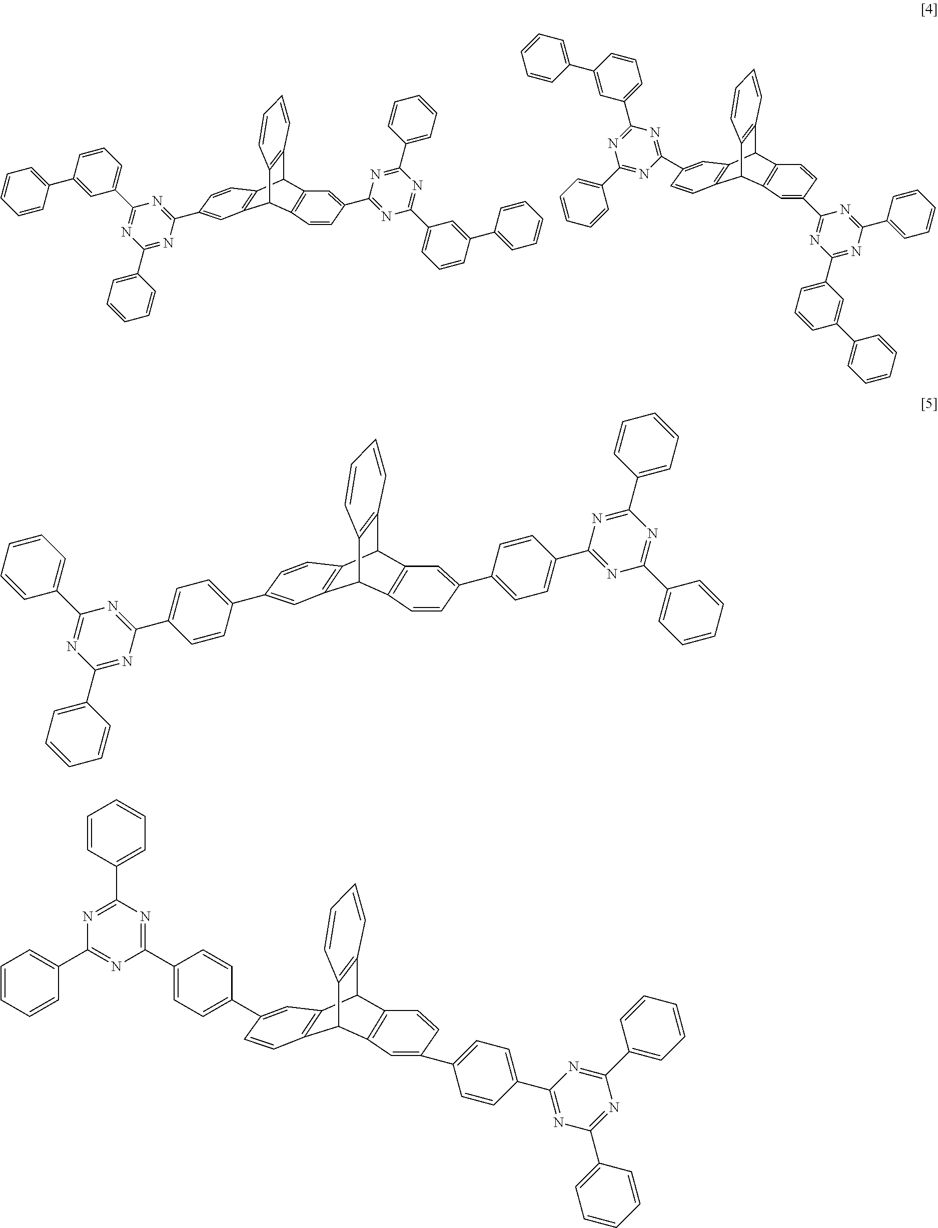

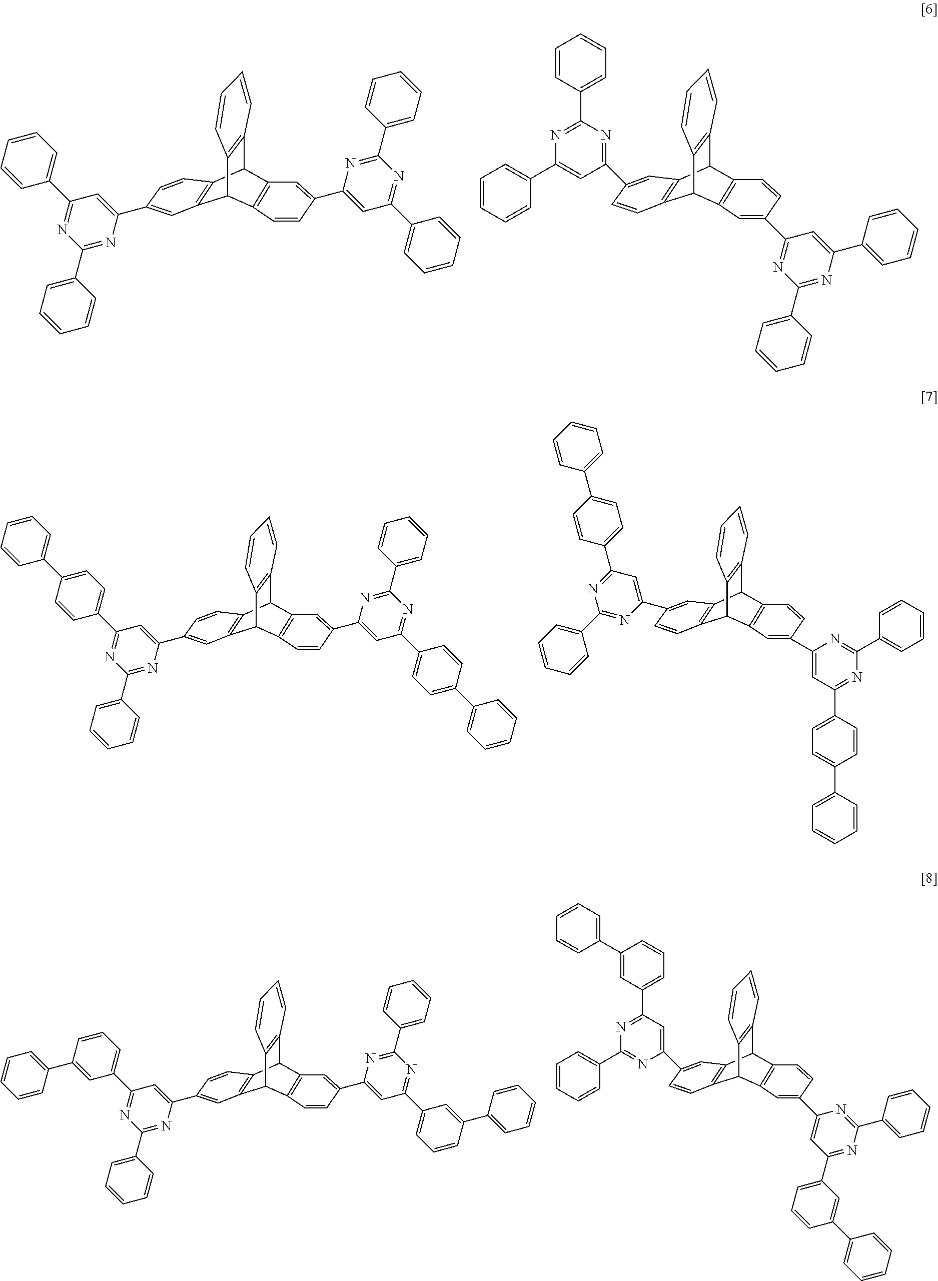

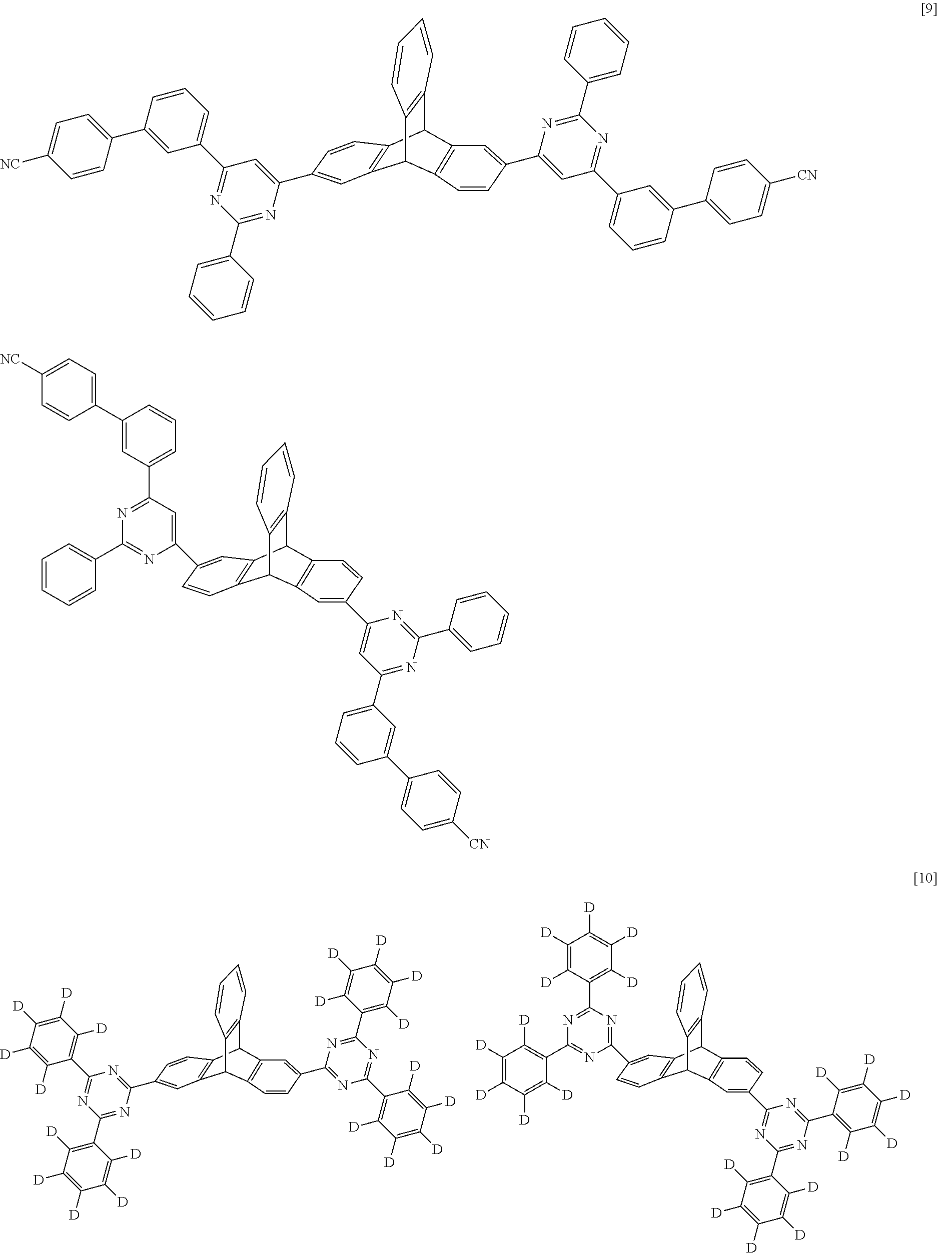

### Third Exemplary Embodiment

### Material for Organic Electroluminescence Device

A material for organic EL device according to a third exemplary embodiment includes the compound according to the first exemplary embodiment. For example, the material for organic EL device may include only the compound according to the first exemplary embodiment. In another case, the material for organic EL device may include the compound according to the first exemplary embodiment and a compound other than the compound according to the first exemplary embodiment. The other compound may be, but is not necessarily, an enantiomer of the compound according to the first exemplary embodiment.

The material for organic EL device according to the third exemplary embodiment can be suitably used as a material for an electron transporting zone (preferably, a material for an electron transporting layer) of an organic EL device.

According to the third exemplary embodiment, a material for organic EL device capable of enhancing the performance of an organic EL device and, in particular, a material for organic EL device capable of reducing the drive voltage of an organic EL device can be provided.

### Fourth Exemplary Embodiment

### Material for Organic Electroluminescence Device

A material for organic EL device according to a fourth exemplary embodiment includes the mixture according to the second exemplary embodiment. For example, the material for organic EL device may include only the mixture according to the second exemplary embodiment. In another case, the material for organic EL device may include the mixture according to the second exemplary embodiment and another compound. Examples of the another compound include any other compound than the first and second compounds included in the mixture according to the second exemplary embodiment.

The material for organic EL device according to the fourth exemplary embodiment can be suitably used as a material for an electron transporting zone (preferably, a material for an electron transporting layer) of an organic EL device.

According to the fourth exemplary embodiment, a material for organic EL device capable of enhancing the performance of an organic EL device and, in particular, a material for organic EL device capable of reducing the drive voltage of an organic EL device can be provided.

### Fifth Exemplary Embodiment

### Organic Electroluminescence Device

An organic EL device according to a fifth exemplary embodiment includes a cathode, an anode, and an organic layer interposed between the cathode and the anode. The organic layer includes at least one layer formed from an organic compound. In another case, the organic layer is formed by a plurality of layers that are formed from an organic compound and stacked on top of each other. The organic layer may further include an inorganic compound.

It is preferable that the organic EL device according to the fifth exemplary embodiment include one or more organic layers and at least one of the organic layers include the compound according to the first exemplary embodiment as a first compound.

It is also preferable that the organic EL device according to the fifth exemplary embodiment include one or more organic layers and at least one of the organic layers include a second compound that is an enantiomer of the first compound.

It is also preferable that the organic EL device according to the fifth exemplary embodiment include one or more organic layers and at least one of the organic layers include the first compound and the second compound that is an enantiomer of the first compound, that is, the mixture according to the second exemplary embodiment.

The organic layer may be formed by, for example, one emitting layer or may include layer(s) that may be included in an organic EL device. Examples of the layers that may be included in an organic EL device include, but are not limited to, at least one layer selected from the group consisting of a hole injecting layer, a hole transporting layer, an electron injecting layer, an electron transporting layer, an electron blocking layer, and a hole blocking layer.

The compound according to the first exemplary embodiment (first compound), the second compound that is an enantiomer of the first compound, and the mixture according to the second exemplary embodiment can be suitably used as a material for an electron transporting zone, preferably used as a material for an electron transporting layer or a hole blocking layer, and more preferably used as a material for an electron transporting layer, of a fluorescent or phosphorescent organic EL device.

In the organic EL device according to the fifth exemplary embodiment, it is preferable that the organic layer include an emitting layer interposed between the cathode and the anode and an electron transporting layer interposed between the cathode and the emitting layer and that the electron transporting layer include the first compound or the second compound that is an enantiomer of the first compound.

In the organic EL device according to the fifth exemplary embodiment, it is also preferable that the organic layer include an emitting layer interposed between the cathode and the anode and an electron transporting layer interposed between the cathode and the emitting layer and that the electron transporting layer include the first compound and the second compound that is an enantiomer of the first compound, that is, the mixture according to the second exemplary embodiment.

In the organic EL device according to the fifth exemplary embodiment, it is preferable that the electron transporting layer include a first electron transporting layer interposed between the cathode and the emitting layer and a second electron transporting layer interposed between the first electron transporting layer and the cathode and that the second electron transporting layer include the first compound or the second compound that is an enantiomer of the first compound.

In the organic EL device according to the fifth exemplary embodiment, it is also preferable that the electron transporting layer include a first electron transporting layer interposed between the cathode and the emitting layer and a second electron transporting layer interposed between the first electron transporting layer and the cathode and that the second electron transporting layer include the first compound and the second compound that is an enantiomer of the first compound, that is, the mixture according to the second exemplary embodiment.

The organic EL device according to the fifth exemplary embodiment may be a fluorescent or phosphorescent monochromatic emitting device or a fluorescent-phosphorescent hybrid white emitting device. The organic EL device according to the fifth exemplary embodiment may be a simple organic EL device including a single emitting unit or a tandem organic EL device including a plurality of emitting units. It is particularly preferable that the organic EL device according to the fifth exemplary embodiment be a fluorescent device. The term “emitting unit” used herein refers to the organic layers. At least one of the organic layers is the emitting layer. The emitting unit is a minimum unit that emits light upon the recombination of the holes and electrons injected.

A typical device arrangement of the simple organic EL device is, for example, as follows:

(1) Anode/Emitting Unit (Organic Layers)/Cathode

The emitting unit may be a multilayer emitting unit that includes a plurality of phosphorescent and fluorescent emitting layers. In such a case, a space layer may be interposed between the emitting layers in order to prevent excitons generated in the phosphorescent emitting layer from being diffused into the fluorescent emitting layer. Typical layer arrangements of the simple emitting unit are described below. Note that the parenthesized layers are optional.

(a) (Hole injecting layer/) Hole transporting layer/Fluorescent emitting layer/Electron transporting layer (/Electron injecting layer)

(b) (Hole injecting layer/) Hole transporting layer/Phosphorescent emitting layer/Electron transporting layer (/Electron injecting layer)

(c) (Hole injecting layer/) Hole transporting layer/First fluorescent emitting layer/Second fluorescent emitting layer/Electron transporting layer (/Electron injecting layer)

(d) (Hole injecting layer/) Hole transporting layer/First phosphorescent emitting layer/Second phosphorescent emitting layer/Electron transporting layer (/Electron injecting layer)

(e) (Hole injecting layer/) Hole transporting layer/Phosphorescent emitting layer/Space layer/Fluorescent emitting layer/Electron transporting layer (/Electron injecting layer)

(f) (Hole injecting layer/) Hole transporting layer/First phosphorescent emitting layer/Second phosphorescent emitting layer/Space layer/Fluorescent emitting layer/Electron transporting layer (/Electron injecting layer)

(g) (Hole injecting layer/) Hole transporting layer/First phosphorescent emitting layer/Space layer/Second phosphorescent emitting layer/Space layer/Fluorescent emitting layer/Electron transporting layer (/Electron injecting layer)

(h) (Hole injecting layer/) Hole transporting layer/Phosphorescent emitting layer/Space layer/First fluorescent emitting layer/Second fluorescent emitting layer/Electron transporting layer (/Electron injecting layer)

(i) (Hole injecting layer/) Hole transporting layer/Electron blocking layer/Fluorescent emitting layer/Electron transporting layer (/Electron injecting layer)

(j) (Hole injecting layer/) Hole transporting layer/Electron blocking layer/Phosphorescent emitting layer/Electron transporting layer (/Electron injecting layer)

(k) (Hole injecting layer/) Hole transporting layer/Exciton blocking layer/Fluorescent emitting layer/Electron transporting layer (/Electron injecting layer)

(l) (Hole injecting layer/) Hole transporting layer/Exciton blocking layer/Phosphorescent emitting layer/Electron transporting layer (/Electron injecting layer)

(m) (Hole injecting layer/) First hole transporting layer/Second hole transporting layer/Fluorescent emitting layer/Electron transporting layer (/Electron injecting layer)

(n) (Hole injecting layer/) First hole transporting layer/Second hole transporting layer/Phosphorescent emitting layer/Electron transporting layer (/Electron injecting layer)

(o) (Hole injecting layer/) First hole transporting layer/Second hole transporting layer/Fluorescent emitting layer/First electron transporting layer/Second electron transporting layer (/Electron injecting layer)

(p) (Hole injecting layer/) First hole transporting layer/Second hole transporting layer/Phosphorescent emitting layer/First electron transporting layer/Second electron transporting layer (/Electron injecting layer)

(q) (Hole injecting layer/) Hole transporting layer/Fluorescent emitting layer/Hole blocking layer/Electron transporting layer (/Electron injecting layer)

(r) (Hole injecting layer/) Hole transporting layer/Phosphorescent emitting layer/Hole blocking layer/Electron transporting layer (/Electron injecting layer)

(s) (Hole injecting layer/) Hole transporting layer/Fluorescent emitting layer/Exciton blocking layer/Electron transporting layer (/Electron injecting layer)

(t) (Hole injecting layer/) Hole transporting layer/Phosphorescent emitting layer/Exciton blocking layer/Electron transporting layer (/Electron injecting layer)

The colors of light emitted by the above phosphorescent or fluorescent emitting layers may be mutually different. Specifically, the above emitting unit (f) may have, for example, the following layer arrangement: (Hole injecting layer/) Hole transporting layer/First phosphorescent emitting layer (emits red light)/Second phosphorescent emitting layer (emits green light)/Space layer/Fluorescent emitting layer (emits blue light)/Electron transporting layer.

Optionally, an electron blocking layer may be interposed between each emitting layer and the hole transporting layer or space layer as needed. Further, a hole blocking layer may be interposed between each emitting layer and the electron transporting layer as needed. The electron blocking layer or hole blocking layer enables electrons or holes to be confined in the emitting layer, thereby increasing the probability of charge recombination in the emitting layer, and consequently enhancing luminous efficiency.

A typical device arrangement of the tandem organic EL device is, for example, as follows:

(2) Anode/First Emitting Unit/Intermediate Layer/Second Emitting Unit/Cathode

The first and second emitting units can be each independently selected from, for example, the above-described emitting units.

The intermediate layer is commonly also referred to as an intermediate electrode, intermediate conductive layer, charge generating layer, electron drawing layer, connection layer, or intermediate insulation layer. The intermediate layer may be formed from a known material capable of feeding electrons and holes to the first and second emitting units, respectively.

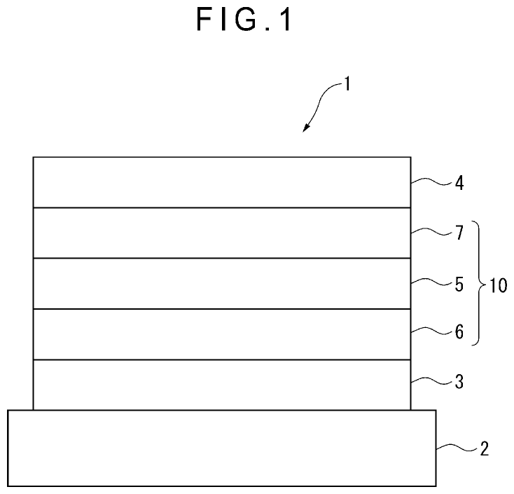

FIG. 1 is a schematic diagram of an exemplary arrangement of the organic EL device according to the invention. An organic EL device 1 includes a substrate 2, an anode 3, a cathode 4, and an emitting unit (organic layers) 10 interposed between the anode 3 and the cathode 4. The emitting unit 10 includes an emitting layer 5. A hole transporting zone 6 (hole injecting layer, hole transporting layer, and the like) is interposed between the emitting layer 5 and the anode 3. An electron transporting zone 7 (electron injecting layer, electron transporting layer, and the like) is interposed between the emitting layer 5 and the cathode 4. Optionally, an electron blocking layer (not illustrated in the drawing) may be provided on a side of the emitting layer 5 close to the anode 3, and a hole blocking layer (not illustrated in the drawing) may be provided on a side of the emitting layer 5 close to the cathode 4. This enables electrons and holes to be confined in the emitting layer 5 and consequently further increases the efficiency with which excitons are generated in the emitting layer 5.

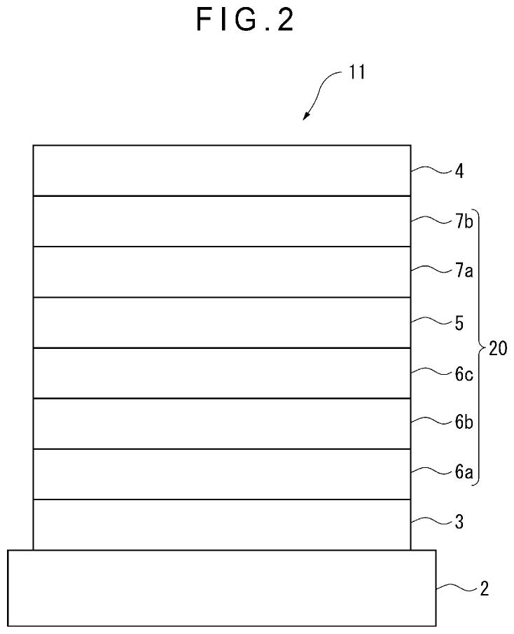

FIG. 2 is a schematic diagram of another exemplary arrangement of the organic EL device according to the invention. An organic EL device 11 includes a substrate 2, an anode 3, a cathode 4, and an emitting unit (organic layers) 20 interposed between the anode 3 and the cathode 4. The emitting unit 20 includes an emitting layer 5. A hole transporting zone interposed between the anode 3 and the emitting layer 5 is formed by a hole injecting layer 6a, a first hole transporting layer 6b, and a second hole transporting layer 6c. An electron transporting zone interposed between the emitting layer 5 and the cathode 4 is formed by a first electron transporting layer 7a and a second electron transporting layer 7b.

In the exemplary embodiment, a host combined with a fluorescent dopant (fluorescent material) is referred to as a fluorescent host, and a host combined with a phosphorescent dopant is referred to as a phosphorescent host. Note that the fluorescent and phosphorescence hosts are not distinguished from each other only by the molecular structure. In other words, the phosphorescence host is a material for forming a phosphorescent emitting layer including a phosphorescent dopant, and it does not mean that the phosphorescence host cannot be used as a material for forming a fluorescent emitting layer. The same applies to the fluorescent host.

The arrangement of the organic EL device is further described below. Hereinafter, reference numerals may be omitted.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of the metal (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Hole Injecting Layer

The hole injecting layer is a layer including a material having high hole injectability and is interposed between the anode and the emitting layer or, when a hole transporting layer is present, between the hole transporting layer and the anode.

Examples of the material having high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

Examples of the highly hole-injectable material further include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,34:20,30-h]quinoxaline-2,3,6,7,10,11-hexacarbonitrile (HAT-CN).

A high polymer compound (e.g., oligomer, dendrimer and polymer) is usable as the material exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide] (abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid)(PAni/PSS) are also usable.

It is also preferable to use an acceptor material, such as a hexaazatriphenylene (HAT) compound represented by a formula (K) below.

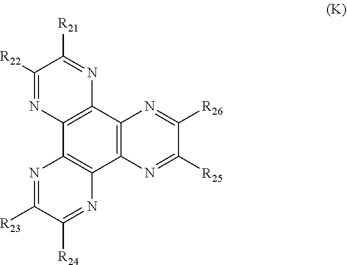

In the formula (K), R₂₁ to R₂₆ each independently represent a cyano group, —CONH₂, a carboxyl group, or —COOR₂₇ (where R₂₇ represents an alkyl group having 1 to 20 carbon atoms or a cycloalkyl group having 3 to 20 carbon atoms); and adjacent two selected from R₂₁ and R₂₂, R₂₃ and R₂₄, and R₂₅ and R₂₆ may be bonded to each other to form a group represented by —CO—O—CO—.

Examples of R₂₇ include a methyl group, an ethyl group, an n-propyl group, an isopropyl group, an n-butyl group, an isobutyl group, a t-butyl group, a cyclopentyl group, and a cyclohexyl group.

### Hole Transporting Layer

The hole transporting layer is a layer including a material having high hole transportability (hole transporting material) and is interposed between the anode and the emitting layer or, when a hole injecting layer is present, between the hole injecting layer and the emitting layer.

The hole transporting layer may have a single-layer structure or a multilayer structure. For example, the hole transporting layer may have a two-layer structure including a first hole transporting layer (anode side) and a second hole transporting layer (cathode side). In an exemplary arrangement of the exemplary embodiment, the hole transporting layer having a single-layer structure is preferably arranged adjacent to the emitting layer, and one of the hole transporting layers included in the multilayer structure closest to the cathode, that is, for example, the second hole transporting layer included in the above two-layer structure, is preferably arranged adjacent to the emitting layer. In another exemplary arrangement of the exemplary embodiment, for example, the electron blocking layer described below may be interposed between the hole transporting layer having a single-layer structure and the emitting layer or between one of the hole transporting layers included in the multilayer structure closest to the emitting layer and the emitting layer.

An aromatic amine compound, carbazole derivative, anthracene derivative and the like are usable for the hole transporting layer.

Examples of the aromatic amine compound include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V.$) or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

A high polymer compound such as poly(N-vinylcarbazole) (abbreviation: PVK) and poly(4-vinyltriphenylamine) (abbreviation: PVTPA) is also usable.

Note that a compound other than the above-described one may be also used when the other compound has hole transportability higher than electron transportability.

### Dopant Material for Emitting Layer

The emitting layer is a layer including a highly emittable material (dopant material). Various materials can be used. For example, a fluorescent material and a phosphorescent material can be used as a dopant material. The fluorescent material is a compound that emits light in a singlet state. The phosphorescent material is a compound that emits light in a triplet state.

Examples of a blue fluorescent material usable for the emitting layer include a pyrene derivative, styrylamine derivative, chrysene derivative, fluoranthene derivative, fluorene derivative, diamine derivative, and triarylamine derivative. Specific examples include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green fluorescent material usable for the emitting layer include an aromatic amine derivative. Specific examples of the green fluorescent material include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N-triphenyl-1,4-phenylenediamine (abbreviation: 2DPABPhA), N-[9,10-bis(1,1′-biphenyl-2-yl)]—N-[4-(9H-carbazole-9-yl)phenyl]-N-phenylanthracene-2-amine (abbreviation: 2YGABPhA), N,N,9-triphenylanthracene-9-amine (abbreviation: DPhAPhA).

Examples of a red fluorescent material usable for the emitting layer include a tetracene derivative and a diamine derivative. Specific examples include N,N,N′,N′-tetrakis(4-methylphenyl)tetracene-5,11-diamine (abbreviation: p-mPhTD), and 7,14-diphenyl-N,N,N′,N-tetrakis(4-methylphenyl)acenaphtho[1,2-a]fluoranthene-3,10-diamine (abbreviation: p-m PhAFD).

Examples of a blue phosphorescent material usable for the emitting layer include metal complexes such as an iridium complex, osmium complex and platinum complex. Specific examples include bis[2-(4′,6′-difluorophenyl)pyridinato-N,C2liridium(III)tetrakis(1-pyrazolyl)borate (abbreviation: FIr6), bis[2-(4′,6′-difluorophenyl)pyridinato-N,C2′]iridium(III)picolinate (abbreviation: Flrpic), bis[2-(3′,5′bistrifluoromethylphenyl)pyridinato-N,C2liridium(III)picolinate (abbreviation: Ir(CF3ppy)2(pic)), and bis[2-(4′,6′-difluorophenyl)pyridinato-N,C2liridium(III)acetylacetonato (abbreviation: Flracac).

Examples of a green phosphorescent material usable for the emitting layer include an iridium complex. Specific examples include tris(2-phenylpyridinato-N,C2′)iridium(III) (abbreviation: Ir(ppy)₃), bis(2-phenylpyridinato-N,C2′)iridium(III)acetylacetonato (abbreviation: Ir(ppy)₂(acac)), bis(1,2-diphenyl-1H-benzimidazolato)iridium(III)acetylacetonato (abbreviation: Ir(pbi)₂(acac)), and bis(benzo[h]quinolinato)iridium(III)acetylacetonato (abbreviation: Ir(bzq)₂(acac)). Examples of a red phosphorescent material usable for the emitting layer include metal complexes such as an iridium complex, platinum complex, terbium complex, and europium complex. Specific examples include organic metal complexes such as bis[2-(2′-benzo[4,5-α]thienyl)pyridinato-N,C3liridium(III)acetylacetonato (abbreviation: Ir(btp)₂(acac)), bis(1-phenylisoquinolinato-N,C2′)iridium(III)acetylacetonato (abbreviation: Ir(piq)₂(acac)), (acetylacetonato)bis[2,3-bis(4-fluorophenyl)quinolinato]iridium(III) (abbreviation: Ir(Fdpq)₂(acac)), and 2,3,7,8,12,13,17,18-octaethyl-21H,23H-porphyrinplatinum(II) (abbreviation: PtOEP).

Moreover, since a rare-earth metal complex, examples of which include tris(acetylacetonato)(monophenanthroline)terbium(III) (abbreviation: Tb(acac)₃(Phen)), tris(1,3-diphenyl-1,3-propanedionatodionato)(monophenanthroline)europium(III) (abbreviation: Eu(DBM)₃(Phen)), and tris[1-(2-thenoyl)-3,3,3-trifluoroacetonato](monophenanthroline)europium(III) (abbreviation: Eu(TTA)₃(Phen)), emits light from rare-earth metal ions (electron transition between different multiplicities), the rare-earth metal complex is usable as a phosphorescent material.

### Host Material For Emitting Layer

The emitting layer may have a structure formed by dispersing the above-described dopant material in another material (host material). It is preferable to use a material having higher Lowest Unoccupied Molecular Orbital (LUMO level) and lower Highest Occupied Molecular Orbital (HOMO level) than the dopant material.

Examples of the host material include:

(1) a metal complex such as an aluminum complex, beryllium complex, or zinc complex;

(2) a heterocyclic compound such as an oxadiazole derivative, benzimidazole derivative, or phenanthroline derivative;

(3) a fused aromatic compound such as a carbazole derivative, anthracene derivative, phenanthrene derivative, pyrene derivative, or chrysene derivative; and

(4) an aromatic amine compound such as a triarylamine derivative or a fused polycylic aromatic amine derivative.

Specific examples include: metal complexes such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);

a heterocyclic compound such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), bathocuproine (abbreviation: BCP);

a fused aromatic compound such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3 ′,3″-(benzene-1,3,5-triyl)tripyrene (abbreviation: TPB3), 9,10-diphenylanthracene (abbreviation: DPAnth), 6,12-dimethoxy-5,11-diphenylchrysene; and

an aromatic amine compound, such as N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 4,4′-bis[N-(1-naphthyl)-N-phenylamino]biphenyl (abbreviation: NPB or a-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB). A plurality of host materials may be used.

A delayed fluorescent (thermally activated delayed fluorescent) compound can be also used as a host material.

In such a case, it is also preferable that the emitting layer include the dopant material and the delayed fluorescent host material.

In an exemplary arrangement of the exemplary embodiment, the emitting layer preferably does not include a phosphorescent metal complex, and preferably does not include a metal complex other than the phosphorescent metal complex.

### Emission Wavelength of Organic EL Device

When the organic EL device according to the exemplary embodiment is driven, a main peak wavelength of light radiated from the organic EL device is preferably in a range from 380 nm to 500 nm, more preferably in a range from 430 nm to 470 nm.

The main peak wavelength of light radiated from the organic EL device is measured as follows. Voltage is applied on the organic EL devices such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the main peak wavelength (unit: nm).

### Content Ratios of Dopant and Host Materials in Emitting Layer

When the emitting layer includes dopant and host materials, the content ratios of the dopant and host materials in the emitting layer preferably fall within, for example, the following ranges.

The content ratio of the host material is preferably in a range from 80 mass % to 99 mass %, more preferably in a range from 90 mass % to 99 mass %, and further preferably in a range from 95 mass % to 99 mass %.

The content ratio of the dopant material is preferably in a range from 1 mass % to 10 mass %, more preferably in a range from 1 mass % to 7 mass %, and further preferably in a range from 1 mass % to 5 mass %.

The upper limit of the total of the content ratios of the dopant and host materials in the emitting layer is 100 mass %.

In particular, in a case of a blue fluorescent device, anthracene compounds described below are preferably used as the host material.

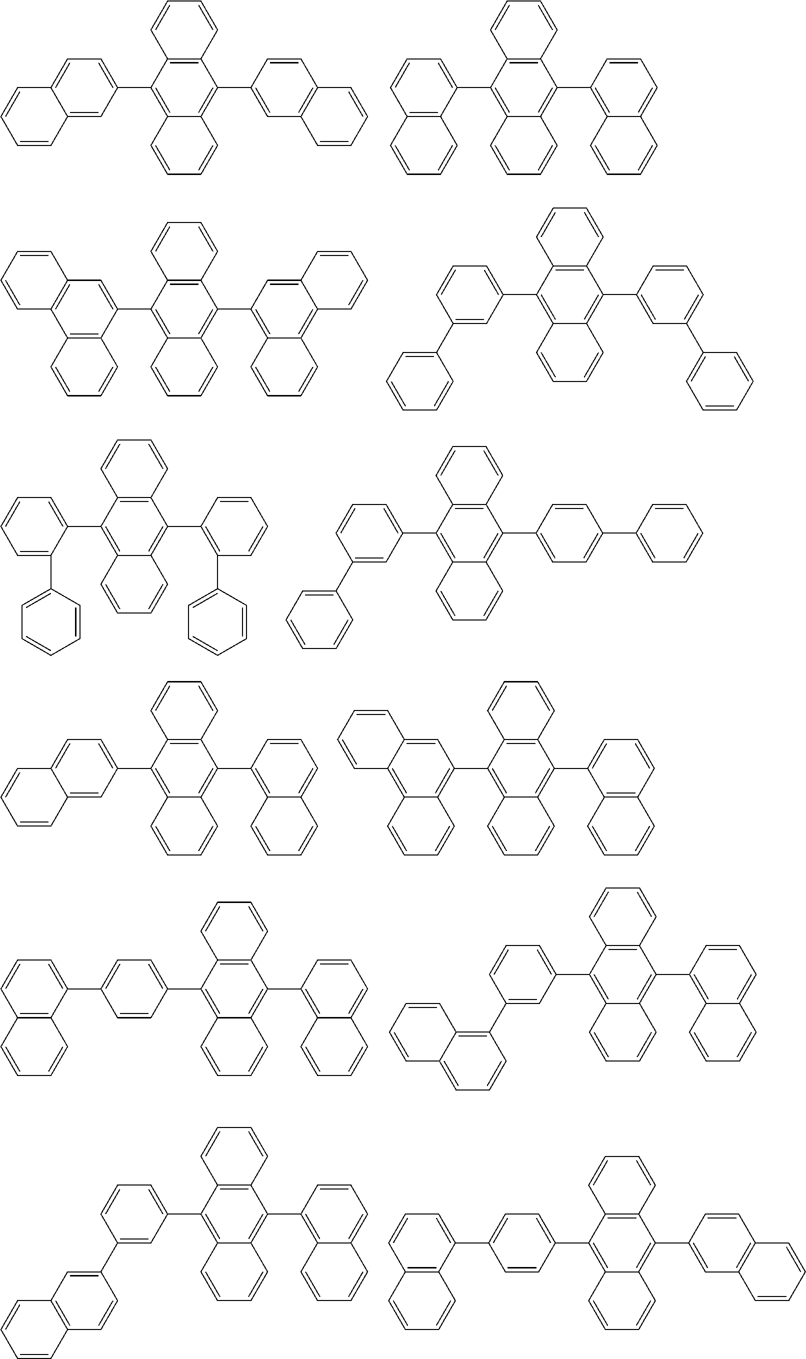

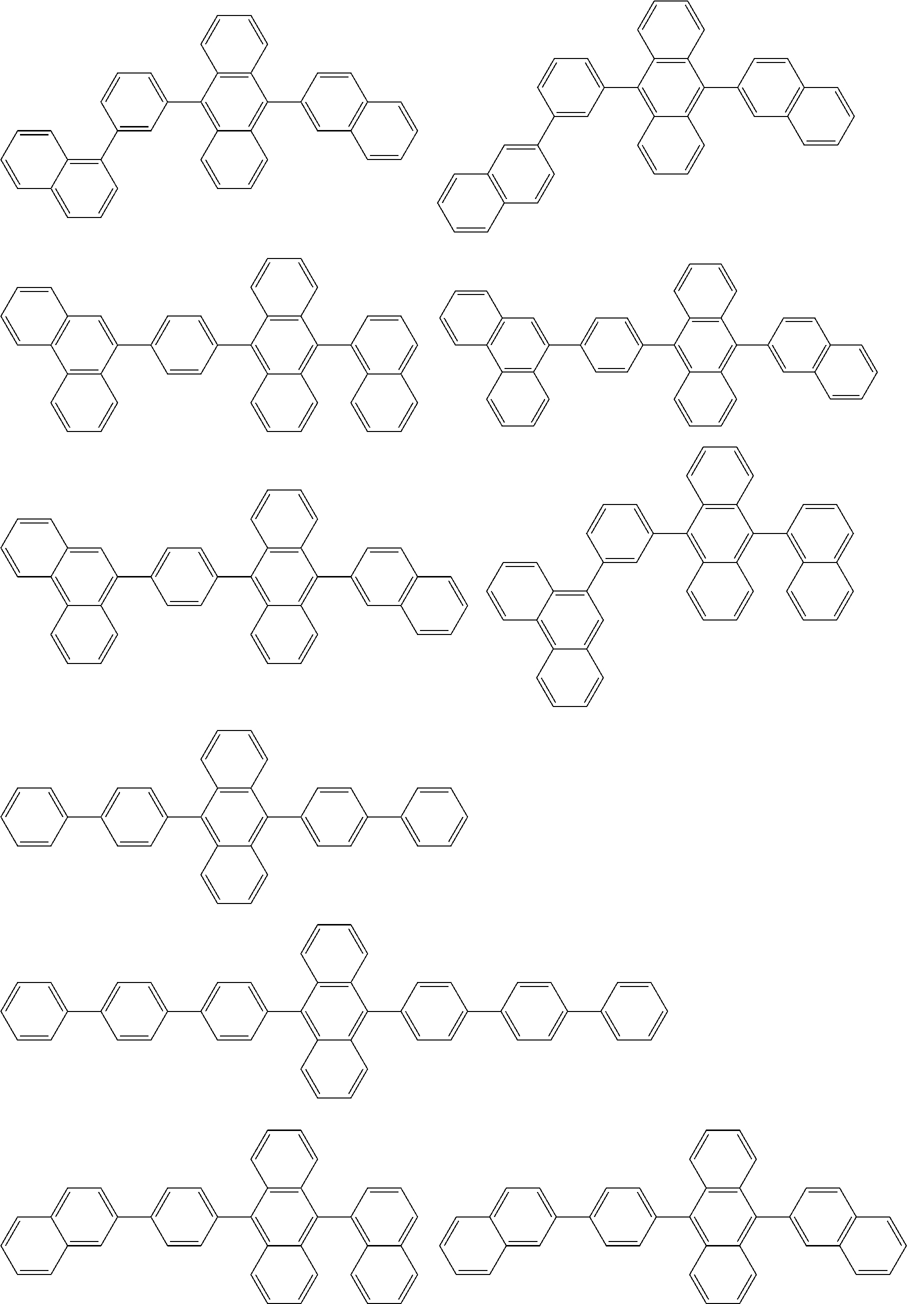

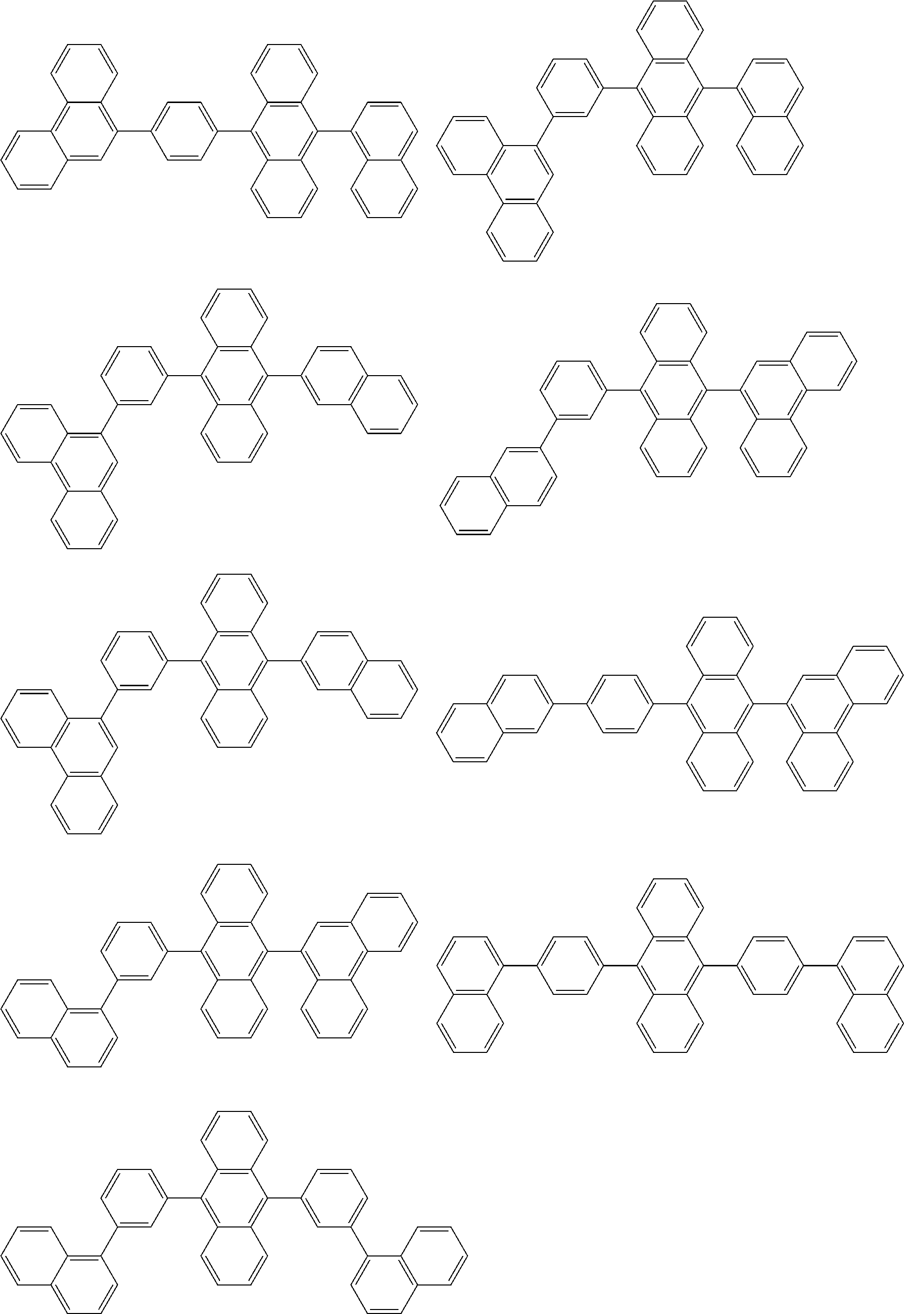

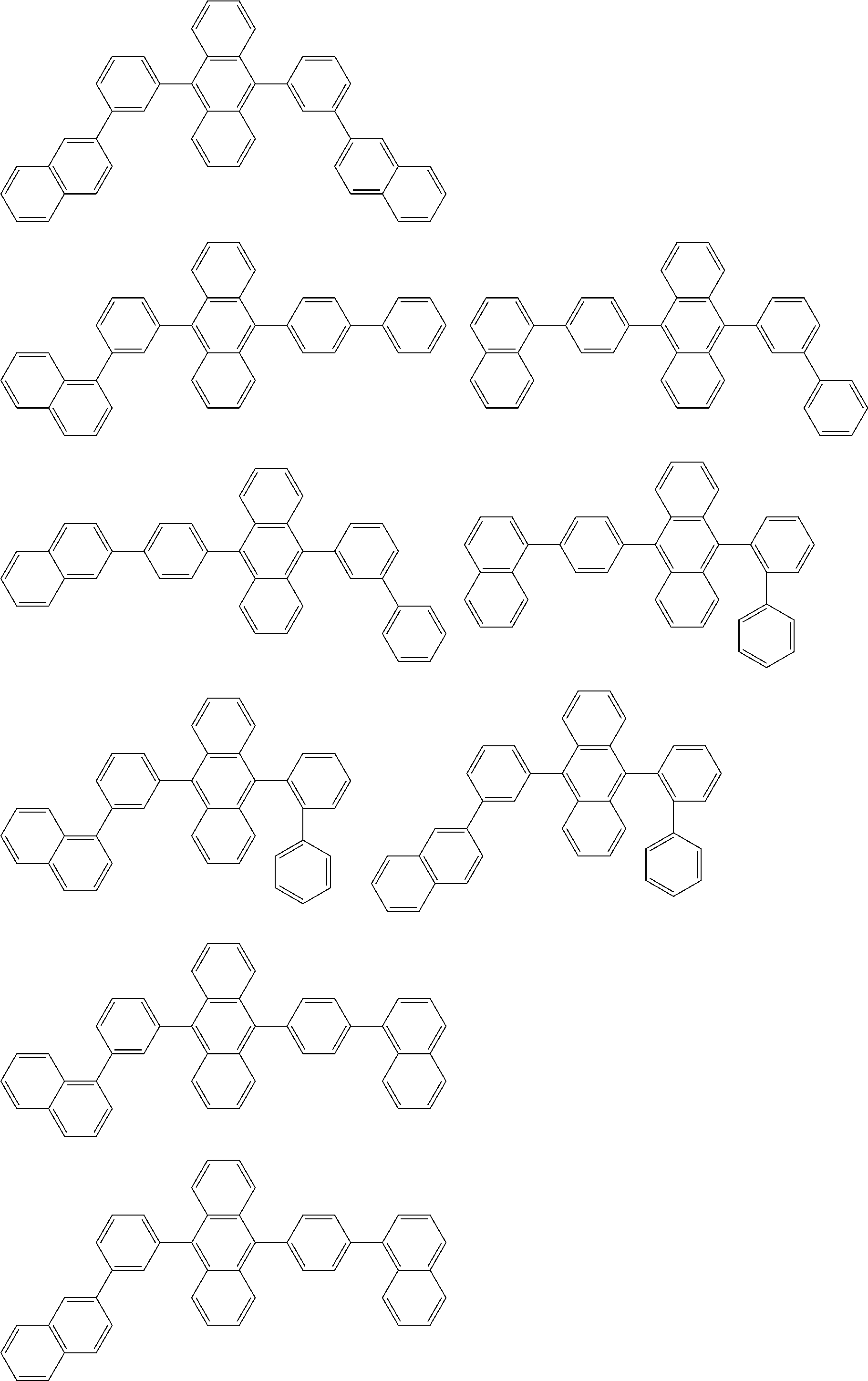

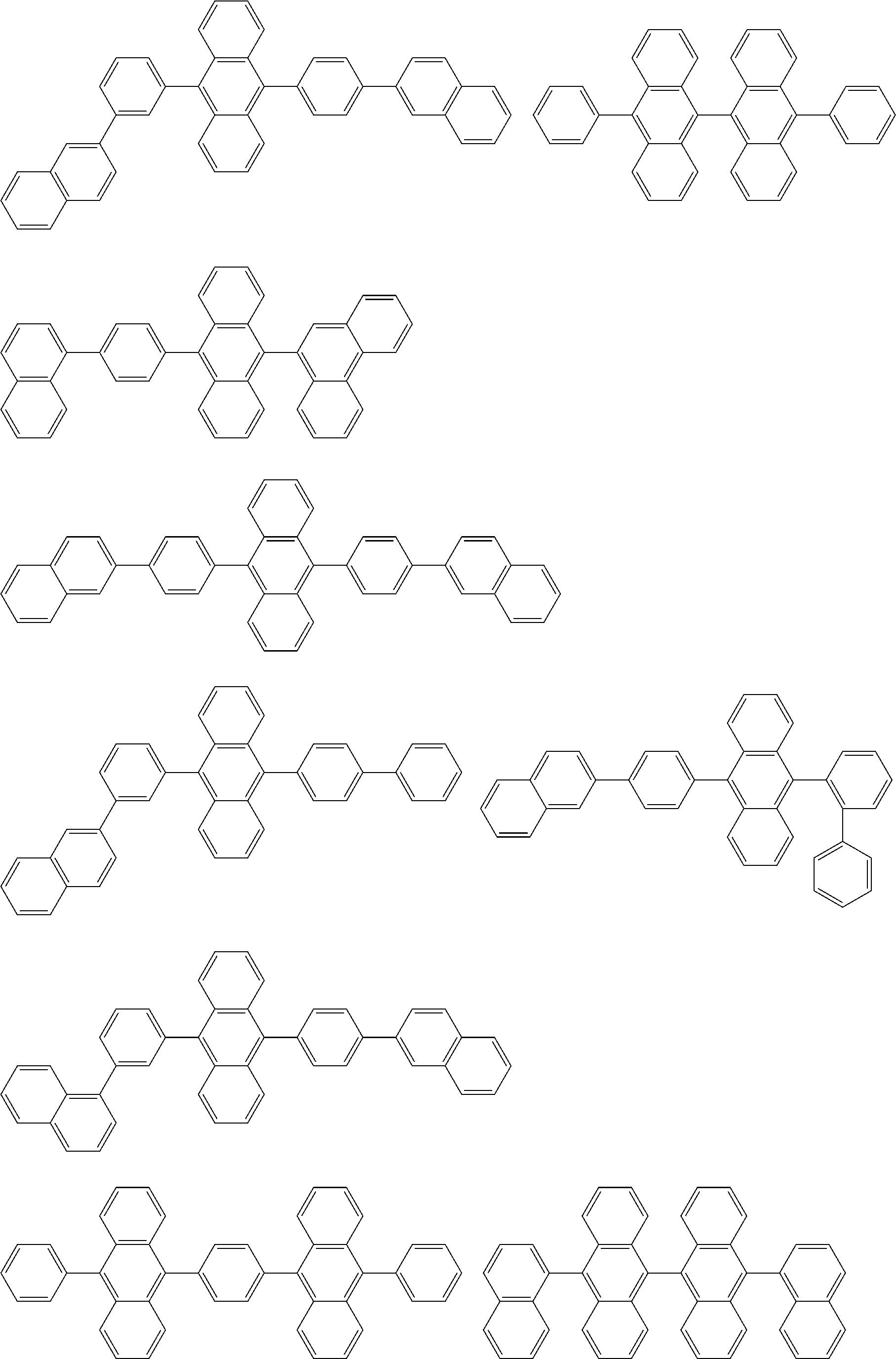

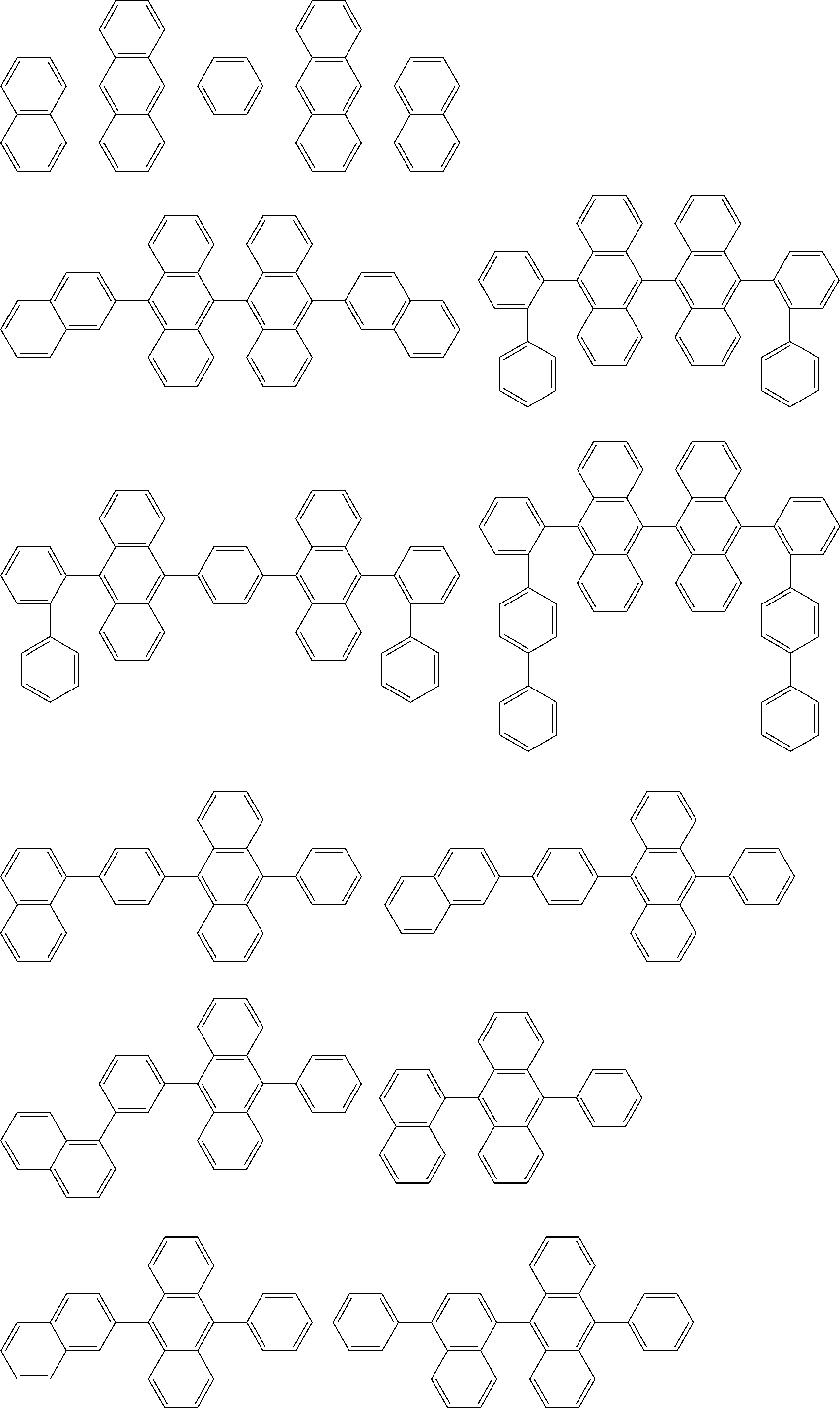

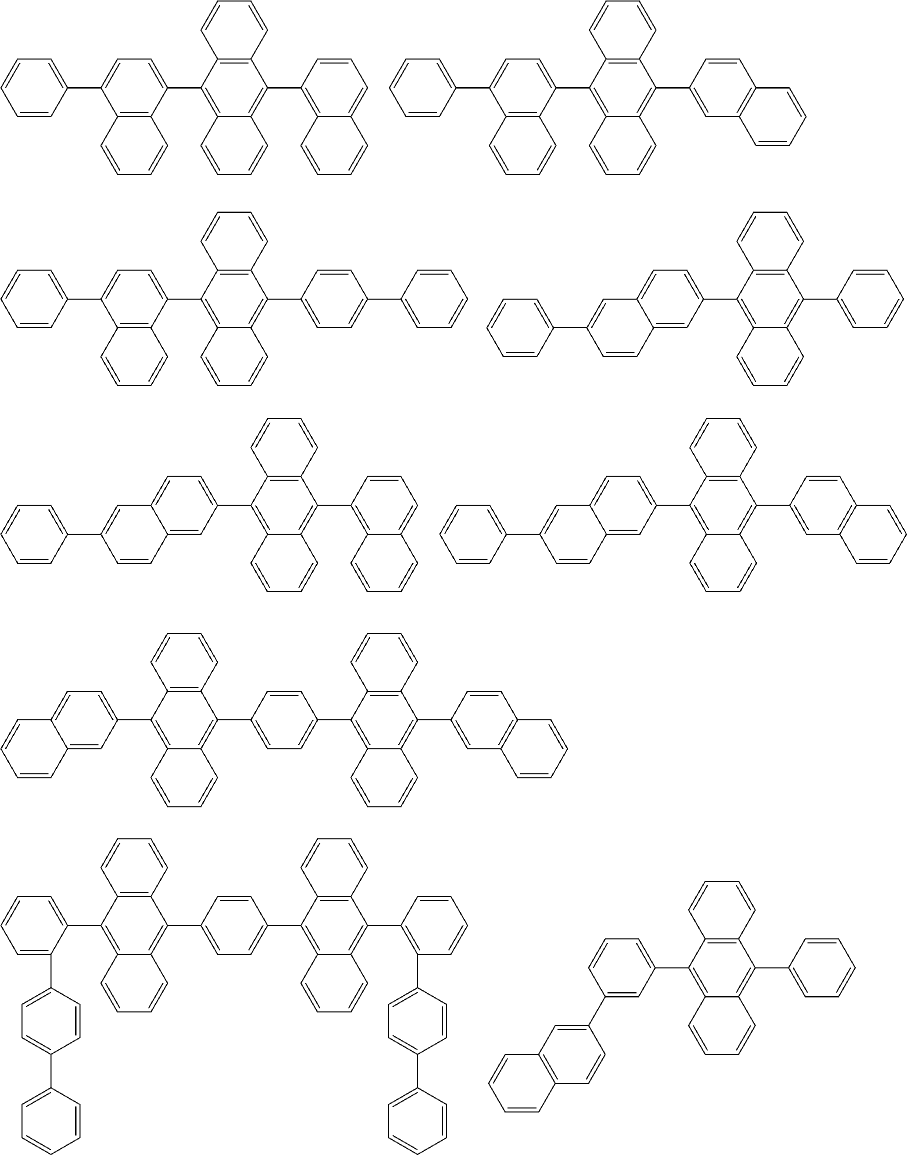

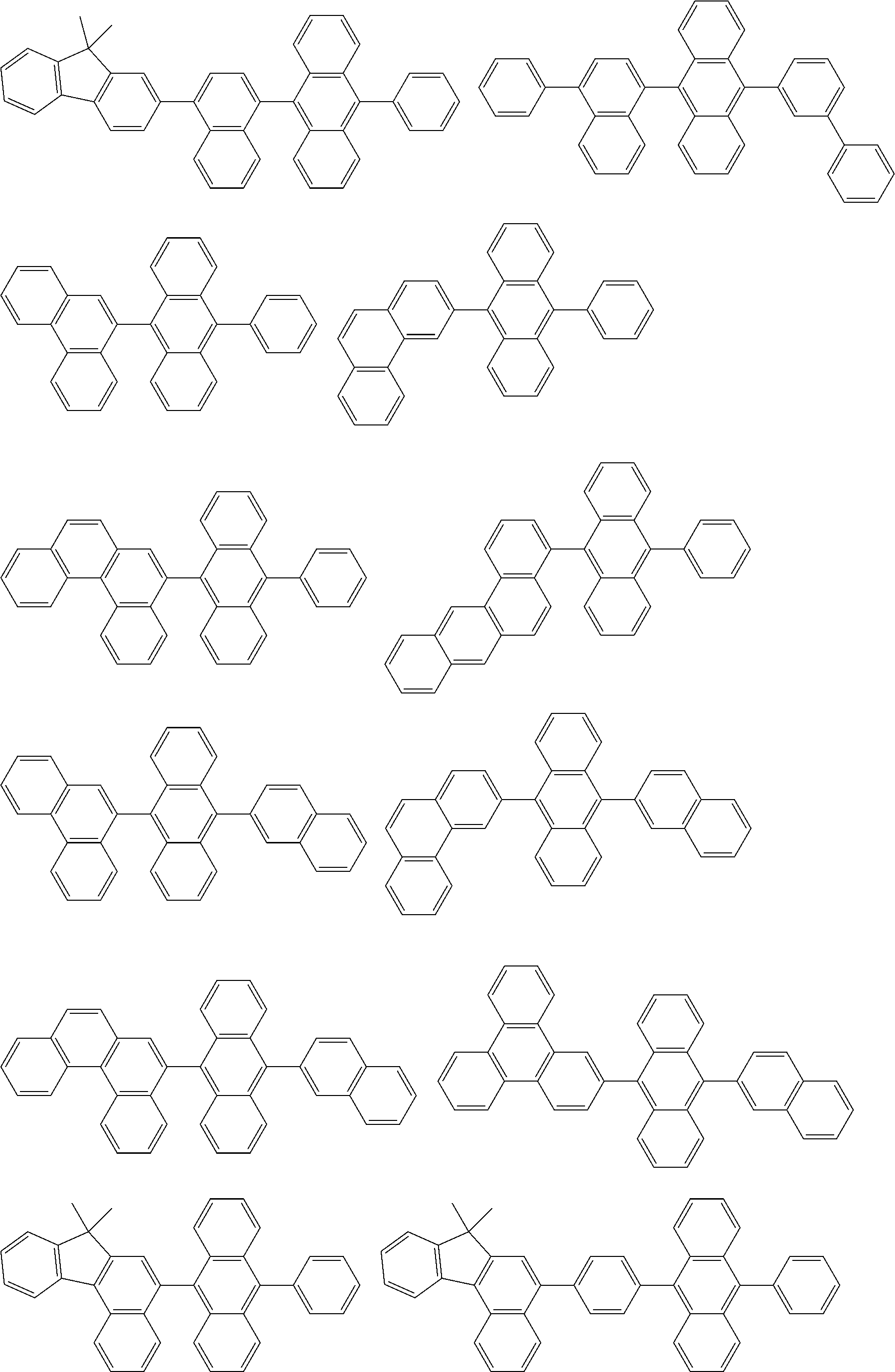

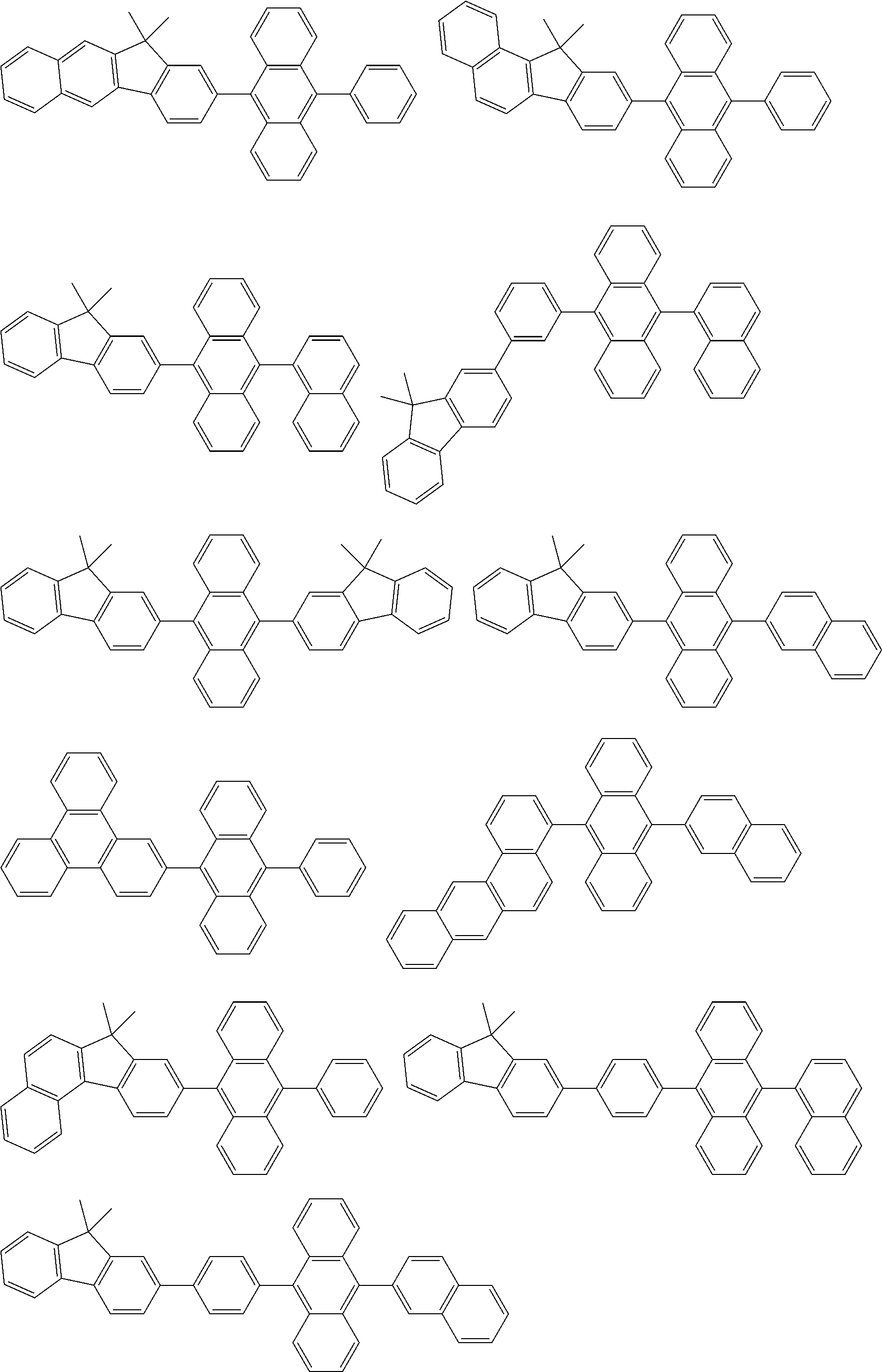

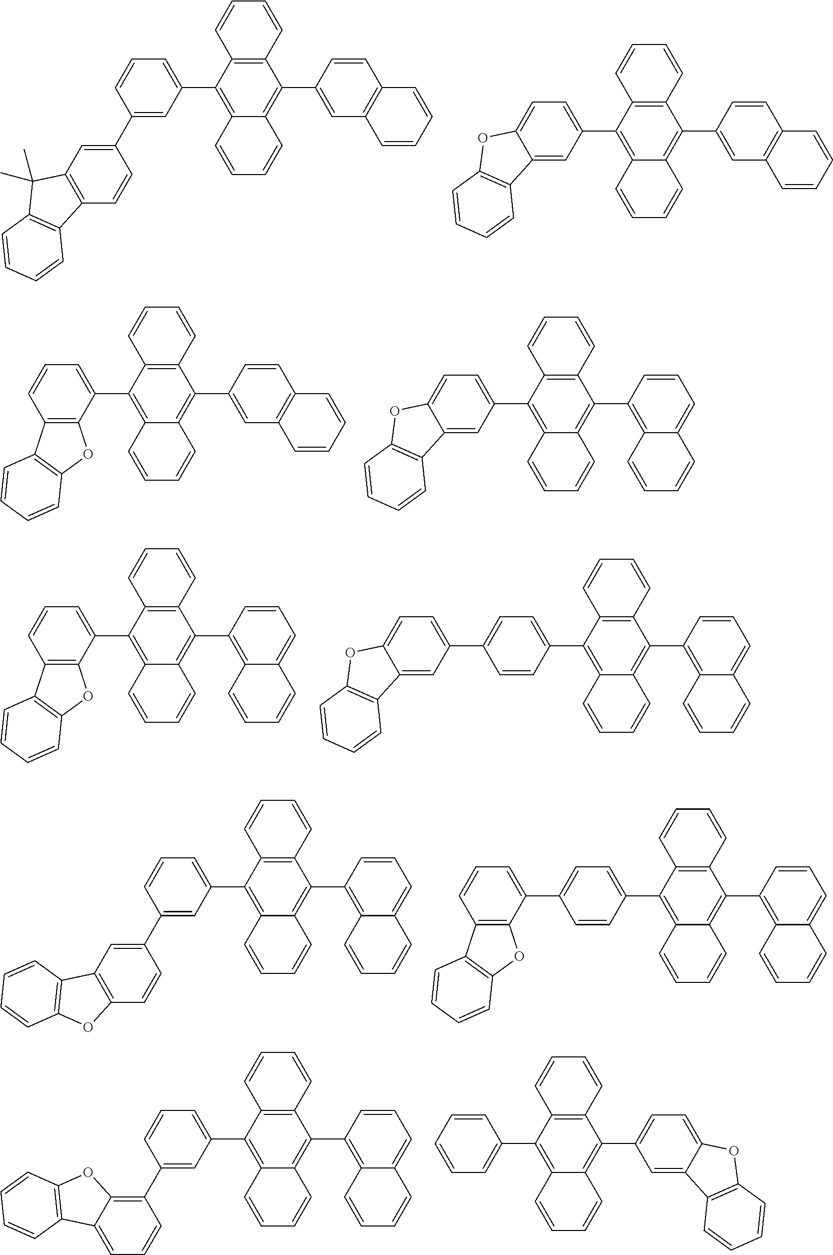

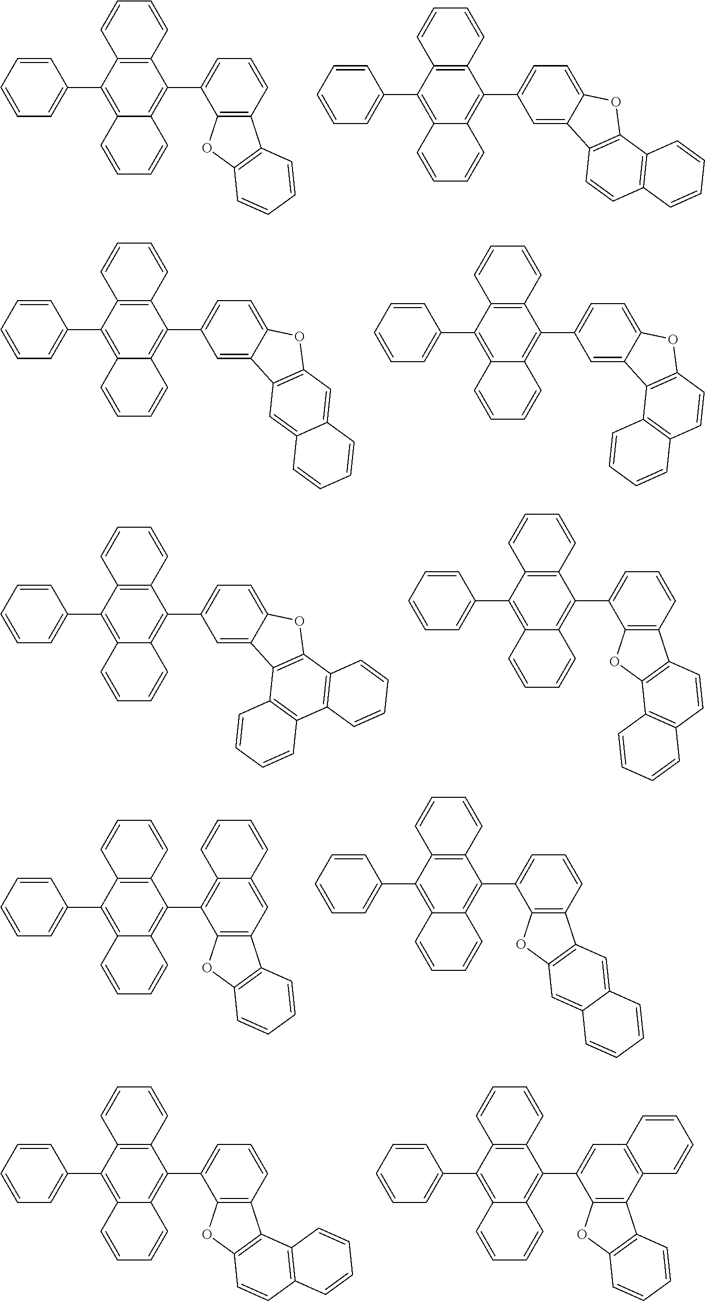

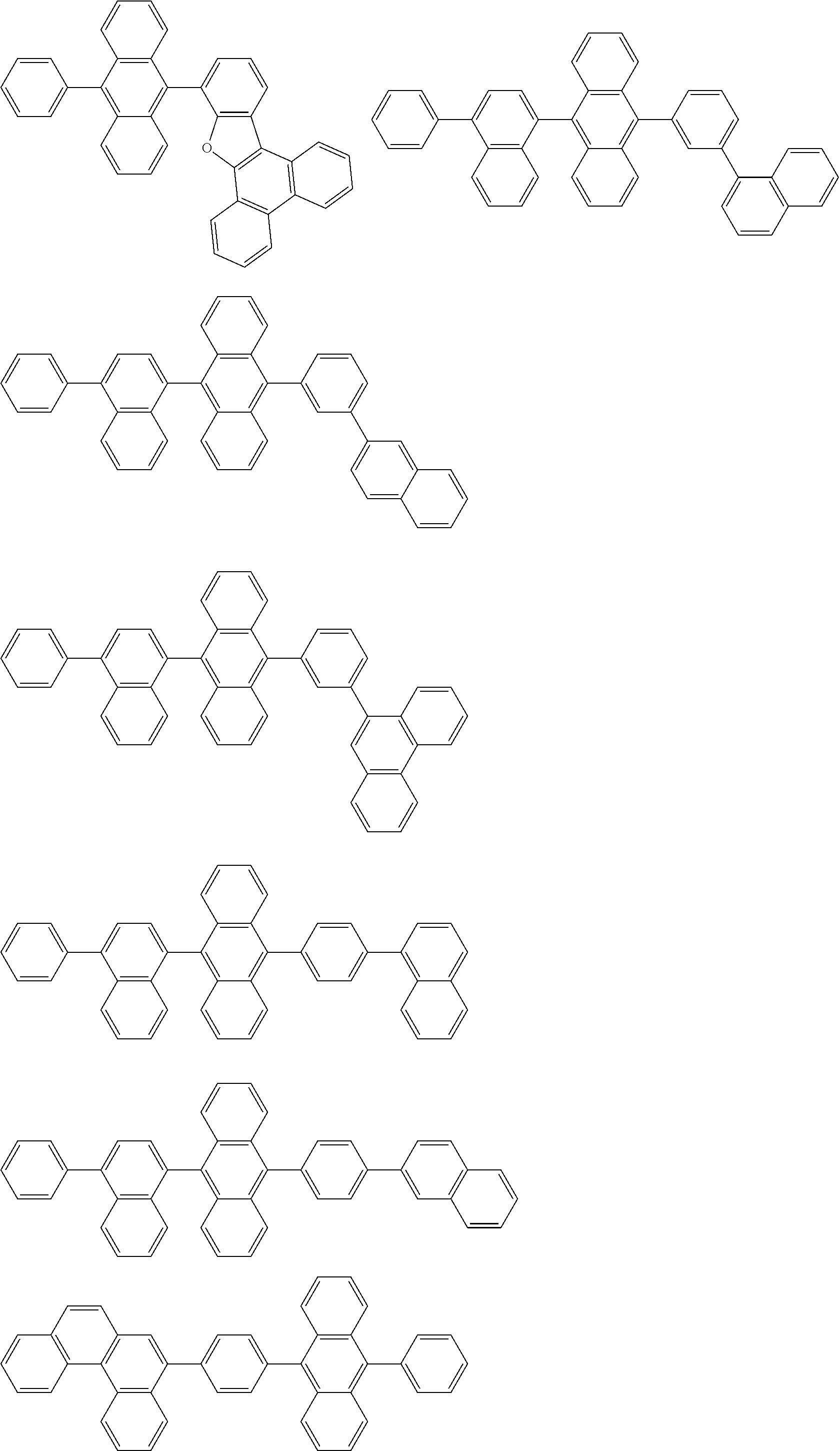

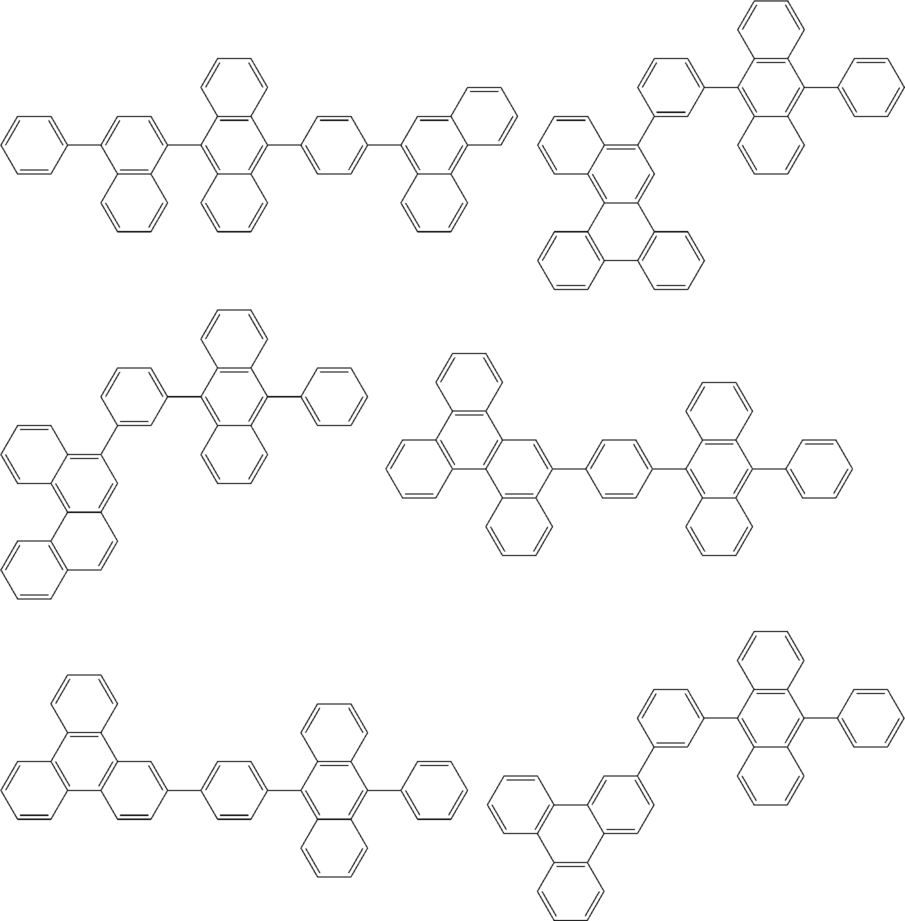

### Electron Transporting Layer

The electron transporting layer is a layer including a material having high electron transportability (electron transporting material) and is interposed between the emitting layer and the cathode or, when an electron injecting layer is present, between the electron injecting layer and the emitting layer.

The electron transporting layer may have a single-layer structure or a multilayer structure. For example, the electron transporting layer may have a two-layer structure including a first electron transporting layer (anode side) and a second electron transporting layer (cathode side). In an exemplary arrangement of the exemplary embodiment, the electron transporting layer having a single-layer structure is preferably arranged adjacent to the emitting layer, and one of the electron transporting layers included in the multilayer structure closest to the anode, that is, for example, the first electron transporting layer included in the above two-layer structure, is preferably arranged adjacent to the emitting layer. In another exemplary arrangement of the exemplary embodiment, for example, the hole blocking layer described below may be interposed between the electron transporting layer having a single-layer structure and the emitting layer or between one of the electron transporting layers included in the multilayer structure closest to the emitting layer and the emitting layer.

Suitable Example in which First Compound is Used

The first compound (the compound according to the first exemplary embodiment) is used as a material for the electron transporting zone, preferably used as a material for the electron injecting layer, the electron transporting layer, the hole blocking layer, or the exciton blocking layer, more preferably used as a material for the electron injecting layer or the electron transporting layer, and further preferably used as a material for the electron transporting layer.

In the above-described electron transporting layer having a two-layer structure, the first compound may be included in one of the first and second electron transporting layers and may be included in both first and second electron transporting layers. In an example of the fifth exemplary embodiment, the first compound is preferably included in only the first electron transporting layer. In another example, the first compound is preferably included in only the second electron transporting layer. In still another example, the first compound is preferably included in both first and second electron transporting layers.

The second compound (the second compound that is an enantiomer of the first compound) and the mixture according to the second exemplary embodiment can be also suitably used as a material for the electron transporting zone.

Examples of the case where the second compound is used as a material for the electron transporting zone include that described in “Suitable Example in Which First Compound Is Used” above in which “first compound” is replaced with “second compound”.

Examples of the case where the mixture according to the second exemplary embodiment is used as a material for the electron transporting zone include that described in “Suitable Example in Which First Compound Is Used” above in which “first compound” is replaced with “mixture according to the second exemplary embodiment”.

In an example of the fifth exemplary embodiment, the first compound included in the organic EL device may include at least one deuterium atom. Or, the first compound may be a mixture of the first compound in which all the hydrogen atoms are protium atoms (hereinafter, referred to as “protium isotope”) and the first compound in which at least one of all the hydrogen atoms is a deuterium atom (deuterium isotope). The protium isotope may include deuterium atoms at a ratio equal to or less than the natural abundance ratio.

In an example of the fifth exemplary embodiment, the first compound included in the electron injecting layer, the electron transporting layer (including the first electron transporting layer, the second electron transporting layer, and the like), the hole blocking layer, and the exciton blocking layer is preferably a protium isotope in consideration of the production costs.

When the organic EL device includes the second compound, the second compound may include at least one deuterium atom. In such a case, the second compound may be a mixture of a protium isotope and a deuterium isotope.

When the organic EL device includes the mixture according to the second exemplary embodiment, at least one of the first compound or second compound included in the mixture may include at least one deuterium atom. In such a case, in the mixture according to the second exemplary embodiment, the first compound may be a mixture of a protium isotope and a deuterium isotope, the second compound may be a mixture of a protium isotope and a deuterium isotope, and both first and second compounds may be a mixture of a protium isotope and a deuterium isotope.

Thus, the organic EL device according to the fifth exemplary embodiment may be an organic EL device in which at least one layer selected from the electron injecting layer, the electron transporting layer, the hole blocking layer, and the exciton blocking layer includes the first compound, second compound, or mixture according to the second exemplary embodiment which is substantially composed only of a protium isotope. The expression “the first compound substantially composed only of a protium isotope” means that the ratio of the content of a protium isotope to the total amount of the first compound is 90 mol % or more, preferably 95 mol % or more, and more preferably 99 mol % or more (each including 100%). The same applies to the expression “the second compound substantially composed only of a protium isotope”. The expression “the mixture according to the second exemplary embodiment which is substantially composed only of a protium isotope” means that the ratio of the content of a protium isotope to the total amount of the first and second compounds is 90 mol % or more, preferably 95 mol % or more, and more preferably 99 mol % or more (each including 100%).

Examples of a material for the electron transporting layer other than the first compound, the second compound, or the mixture according to the second exemplary embodiment include:

(1) a metal complex such as an aluminum complex, beryllium complex, and zinc complex,

(2) a hetero aromatic compound such as imidazole derivative, benzimidazole derivative, azine derivative, carbazole derivative, and phenanthroline derivative, and

(3) a high-molecular compound.

Examples of the metal complex include tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs).

Examples of the high-molecular compound include poly[(9,9-dihexylfluorene-2,7-diyl)-co-(pyridine-3,5-diyl)] (abbreviation: PF-Py) and poly[(9,9-dioctylfluorene-2,7-diyl)-co-(2,2′-bipyridine-6,6′-diyl)] (abbreviation: PF-BPy).

The above materials have an electron mobility of 10⁻⁶ cm²/(V·s) or more. A material other than the above materials may be used for the electron transporting layer as long as the material exhibits a higher electron transportability than the hole transportability.

### Electron Injecting Layer

The electron injecting layer is a layer including a material having high electron injectability. Examples of the material for the electron injecting layer include: an alkali metal such as lithium (Li) and cesium (Cs); an alkaline-earth metal such as magnesium (Mg), calcium (Ca), and strontium (Sr); a rare-earth metal such as europium (Eu) and ytterbium (Yb); and a compound including any of the above metals. Examples of such a compound include an alkali metal oxide, an alkali metal halide, an alkali metal-containing organic complex, an alkaline-earth metal oxide, an alkaline-earth metal halide, an alkaline-earth metal-containing organic complex, a rare-earth metal oxide, a rare-earth metal halide, and a rare-earth metal-containing organic complex. A plurality of the above compounds may be used in a mixture.

In addition, the alkali metal, alkaline earth metal or the compound thereof may be added to the material exhibiting the electron transportability in use. Specifically, for instance, magnesium (Mg) added to Alq may be used. In this case, the electrons can be more efficiently injected from the anode.

Alternatively, the electron injecting layer may be provided by a composite material in a form of a mixture of the organic compound and the electron donor. Such a composite material exhibits excellent electron injectability and electron transportability since the organic compound receives electrons from the electron donor. In this case, the organic compound is preferably a material excellent in transporting the received electrons. Specifically, the above examples (e.g., the metal complex and the hetero aromatic compound) of the material forming the electron transporting layer are usable. As the electron donor, any material exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Cathode

It is preferable to use metal, an alloy, an electroconductive compound, and a mixture thereof, which have a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Insulation Layer

In an organic EL device, pixel defects are likely to occur due to leakage or short circuiting because an electric field is applied to ultrathin films. In order to prevent this, an insulation layer that is an insulative thin film layer may be interposed between a pair of the electrodes.

Examples of the material for the insulation layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or laminate including any of the above materials may be also used.

### Space Layer

The space layer is, for example, a layer interposed between a fluorescent emitting layer and a phosphorescent emitting layer in a case where the fluorescent emitting layer and the phosphorescent emitting layer are stacked on top of each other, in order to prevent the excitons generated in the phosphorescent emitting layer from being diffused into the fluorescent emitting layer or to adjust carrier balance. The space layer may be also interposed between a plurality of phosphorescent emitting layers.

Since the space layer is interposed between the emitting layers, a material for the space layer preferably has both electron transportability and hole transportability. Further, in order to prevent the diffusion of the triplet energy into adjacent phosphorescent emitting layers, the triplet energy is preferably 2.6 eV or more. Examples of the material that can be used for forming the space layer are the same as the above-described examples of the material that can be used for forming the hole transporting layer.

### Blocking Layer

Blocking layers, such as an electron blocking layer, a hole blocking layer, and an exciton blocking layer, may be arranged adjacent to the emitting layer. The electron blocking layer is a layer that prevents electrons from leaking from the emitting layer to the hole transporting layer. The hole blocking layer is a layer that prevents holes from leaking from the emitting layer to the electron transporting layer. The exciton blocking layer prevents the excitons generated in the emitting layer from being diffused into neighboring layers and confines the excitons in the emitting layer.

Each layer included in the organic EL device can be formed by a known method, such as vapor deposition or a coating method. The vapor deposition is exemplified by vacuum deposition and molecular beam epitaxy (MBE)). The coating method is exemplified by a method using a solution of a compound that forms a layer, such as dipping, spin coating, casting, bar coating, and roll coating.

The thickness of each layer is not particularly limited. The thickness is preferably in a range from 5 nm to 10 μm and more preferably in a range from 10 nm to 0.2 μm, because excessively small film thickness is likely to cause defects (e.g. pin holes) and excessively large thickness leads to the necessity of applying high voltage and consequent reduction in efficiency.

According to the fifth exemplary embodiment, an organic electroluminescence device that includes a compound capable of enhancing the performance of the organic EL device and, in particular, a compound capable of reducing the drive voltage of the organic EL device can be provided.

### Sixth Exemplary Embodiment

### Electronic Device

An electronic device according to a sixth exemplary embodiment includes any of the organic EL devices according to the above-described exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

According to the sixth exemplary embodiment, an electronic device including an organic electroluminescence device that includes a compound capable of enhancing the performance of the organic EL device and, in particular, a compound capable of reducing the drive voltage of the organic EL device can be provided.

The organic EL device can be used in an electronic device, such as a display component (e.g., an organic EL panel module), a display of a television, mobile phone, personal computer, or the like, or a light-emitting unit (e.g., a illuminator and a vehicle light).

### EXAMPLES

Examples according to the invention are described below. The invention is not limited to Examples below.

### Compounds

The compounds represented by the formula (1) which were used in Examples 1 to 8 are described below.

In Example 1, a mixture (isomer mixture) of compounds ET-1a and ET-1b was used.

In the following description of Examples, the mixture of the compounds ET-1a and ET-1b is referred to collectively as “ET-1”.

In Example 2, a mixture of compounds ET-2a and ET-2b (isomer mixture: referred to collectively as ET-2) was used. In Example 3, a mixture of compounds ET-3a and ET-3b (isomer mixture: referred to collectively as ET-3) was used. In Example 4, a mixture of the compounds ET-4a and ET-4b (isomer mixture: referred to collectively as ET-4) was used. In Example 5, a mixture of compounds ET-5a and ET-5b (isomer mixture: referred to collectively as ET-5) was used. In Example 6, a mixture of compounds ET-6a and ET-6b (isomer mixture: referred to collectively as ET-6) was used. In Example 7, a mixture of compounds ET-8a and ET-8b (isomer mixture: referred to collectively as ET-8) was used. In Example 8, a mixture of compounds ET-9a and ET-9b (isomer mixture: referred to collectively as ET-9) was used. In Synthesis Example 7, a mixture of compounds ET-7a and ET-7b was synthesized.

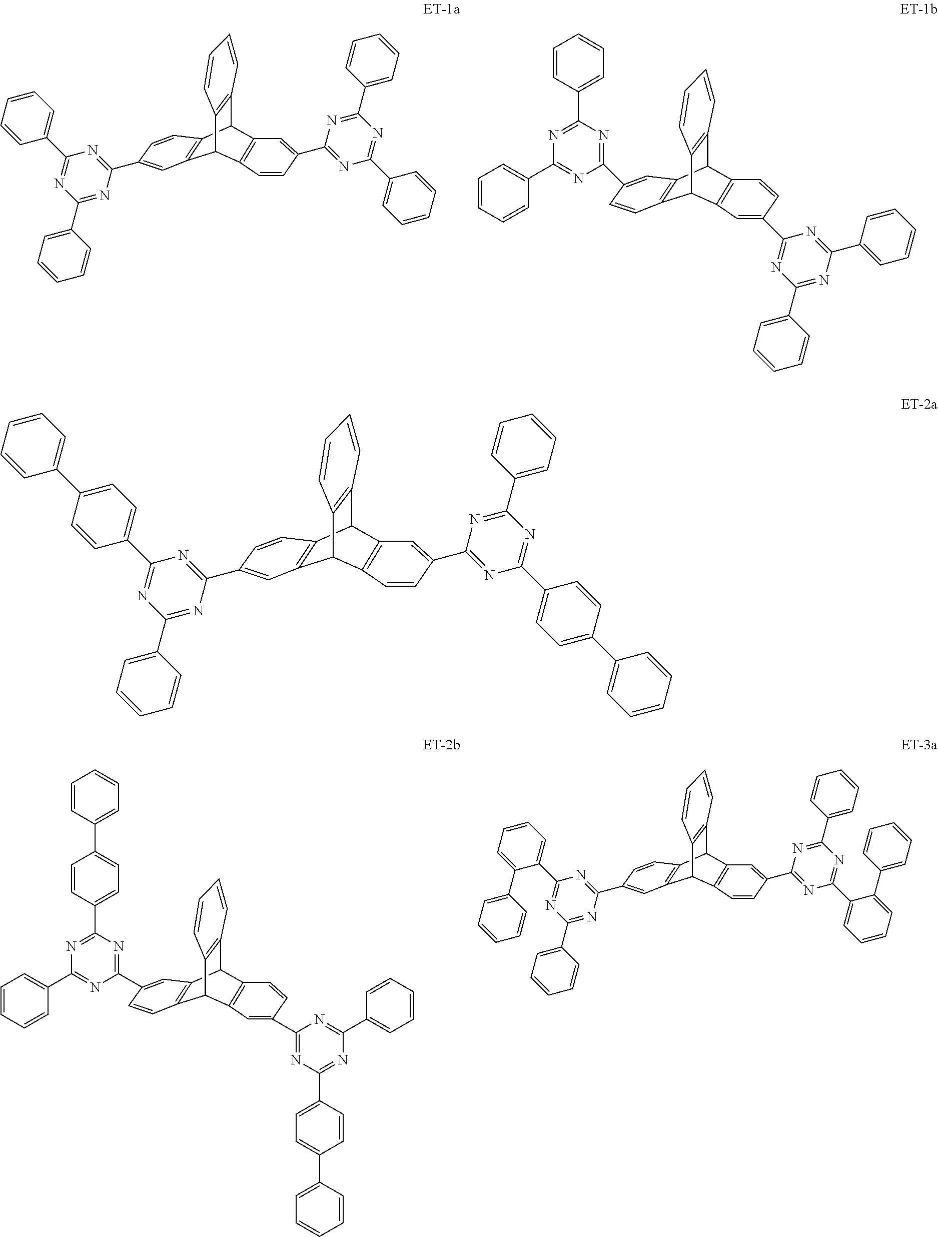

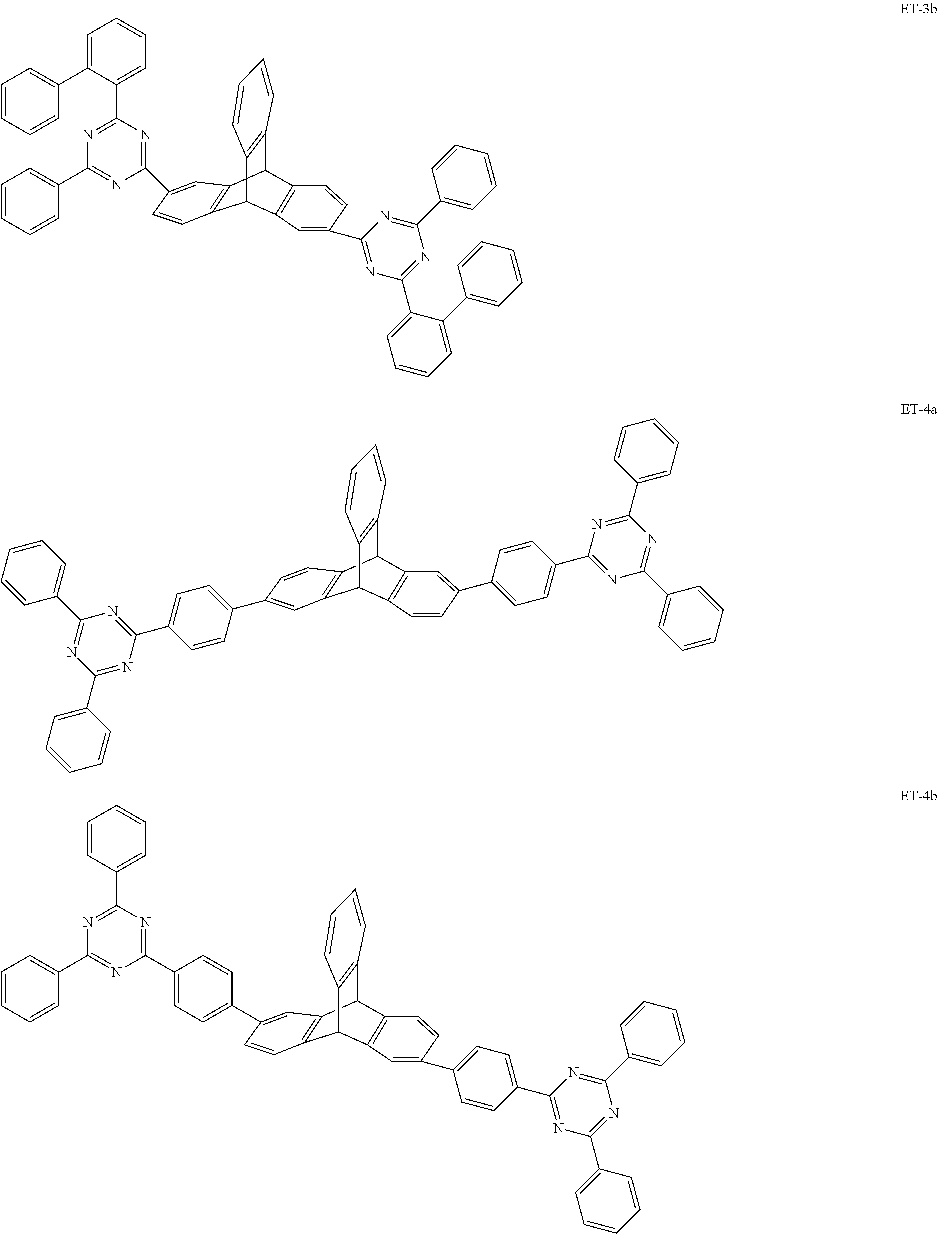

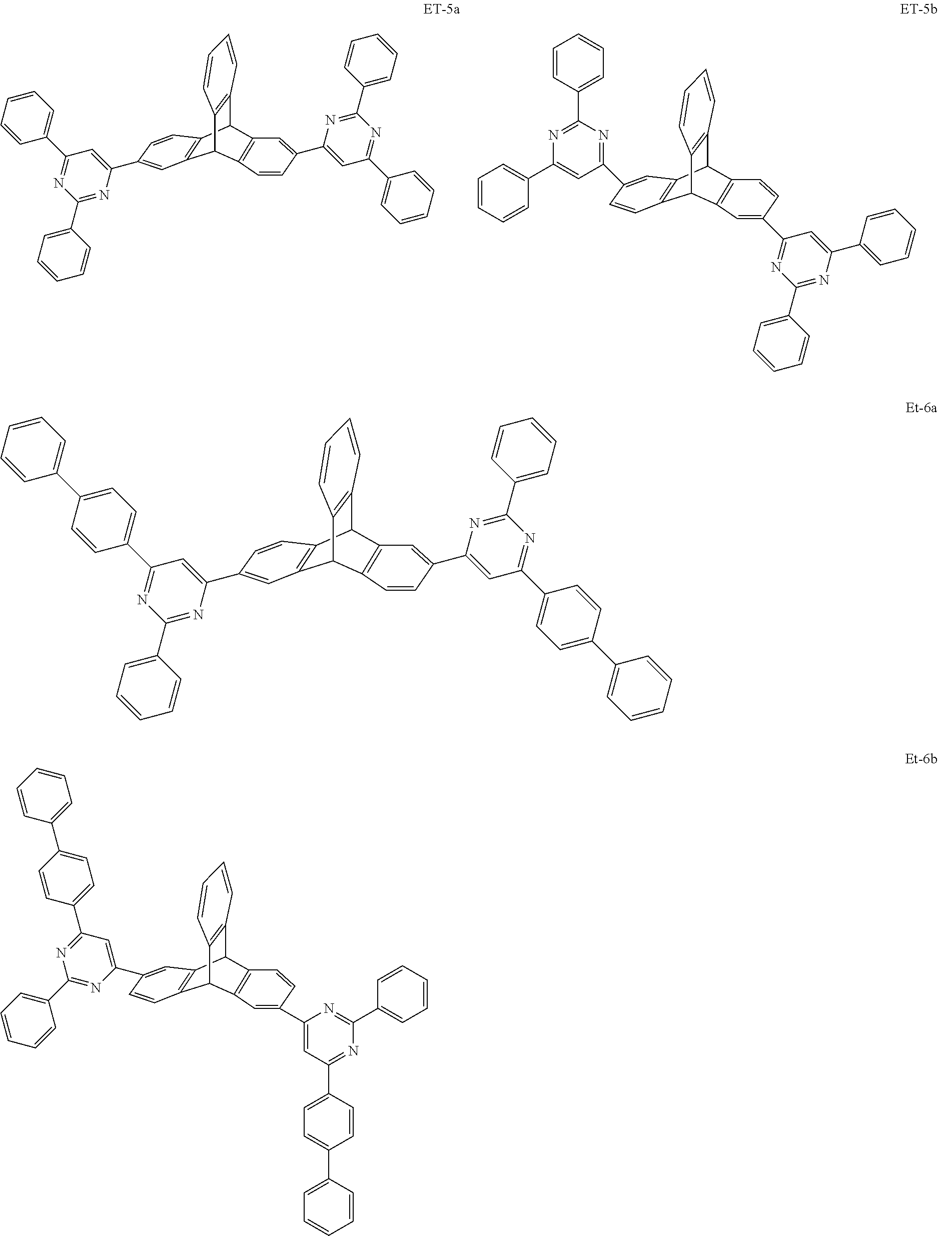 [SMILES: c1ccc(-c2cc(-c3ccc4c(c3)C3c5ccccc5C4c4cc(-c5cc(-c6ccccc6)nc(-c6ccccc6)n5)ccc43)nc(-c3ccccc3)n2)cc1 ; c1ccc(-c2cc(-c3ccc4c(c3)C3c5ccccc5C4c4cc(-c5cc(-c6ccccc6)nc(-c6ccccc6)n5)ccc43)nc(-c3ccccc3)n2)cc1 ; c1ccc(-c2ccc(-c3cc(-c4ccc5c(c4)C4c6ccccc6C5c5cc(-c6cc(-c7ccc(-c8ccccc8)cc7)nc(-c7ccccc7)n6)ccc54)nc(-c4ccccc4)n3)cc2)cc1 ; c1ccc(-c2ccc(-c3cc(-c4ccc5c(c4)C4c6ccccc6C5c5cc(-c6cc(-c7ccc(-c8ccccc8)cc7)nc(-c7ccccc7)n6)ccc54)nc(-c4ccccc4)n3)cc2)cc1]

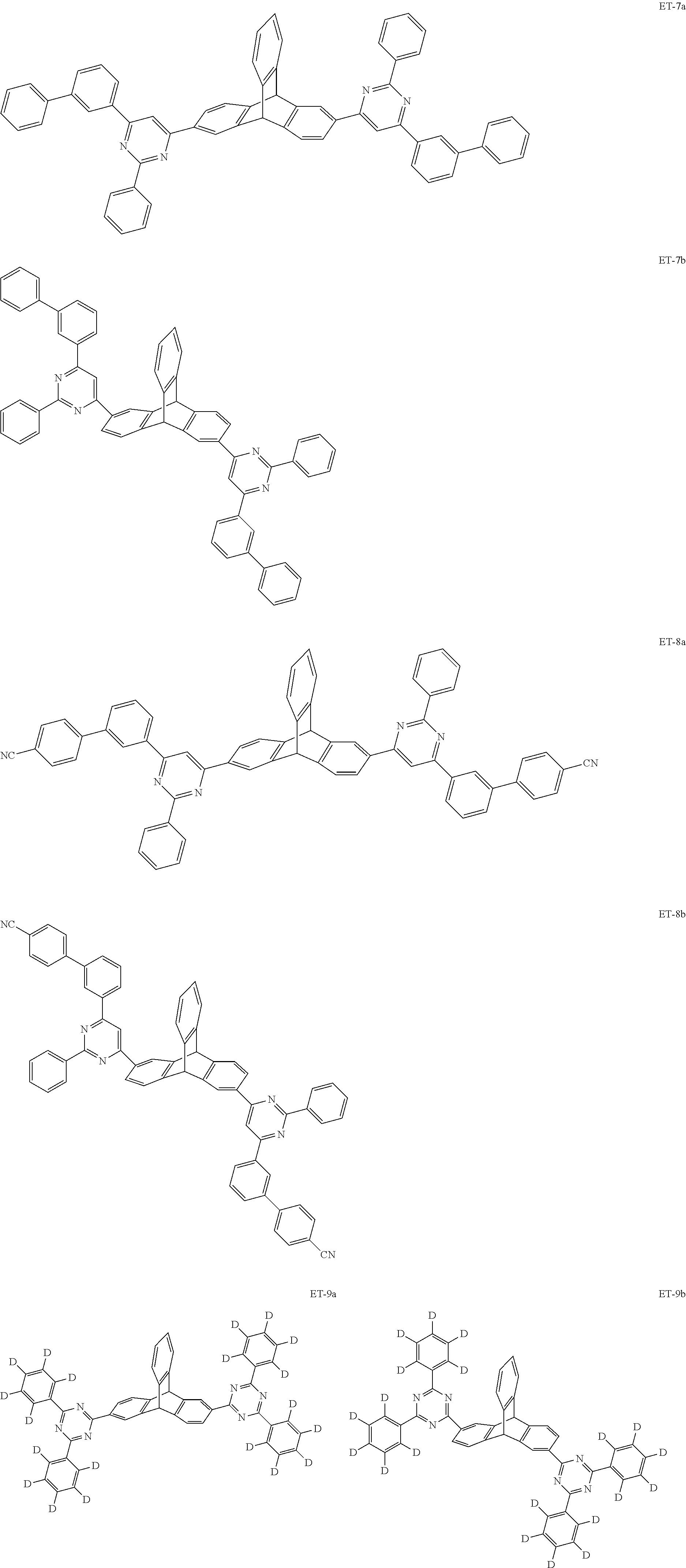

The structure of the compound used for producing an organic EL device of Comparative Example 1 is described below.

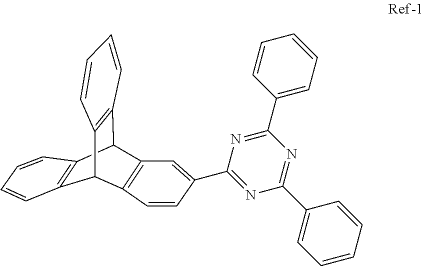 [SMILES: c1ccc(-c2nc(-c3ccccc3)nc(-c3ccc4c(c3)C3c5ccccc5C4c4ccccc43)n2)cc1]

The structures of the other compounds used for producing organic EL devices of Examples 1 to 8 and Comparative Example 1 are described below.

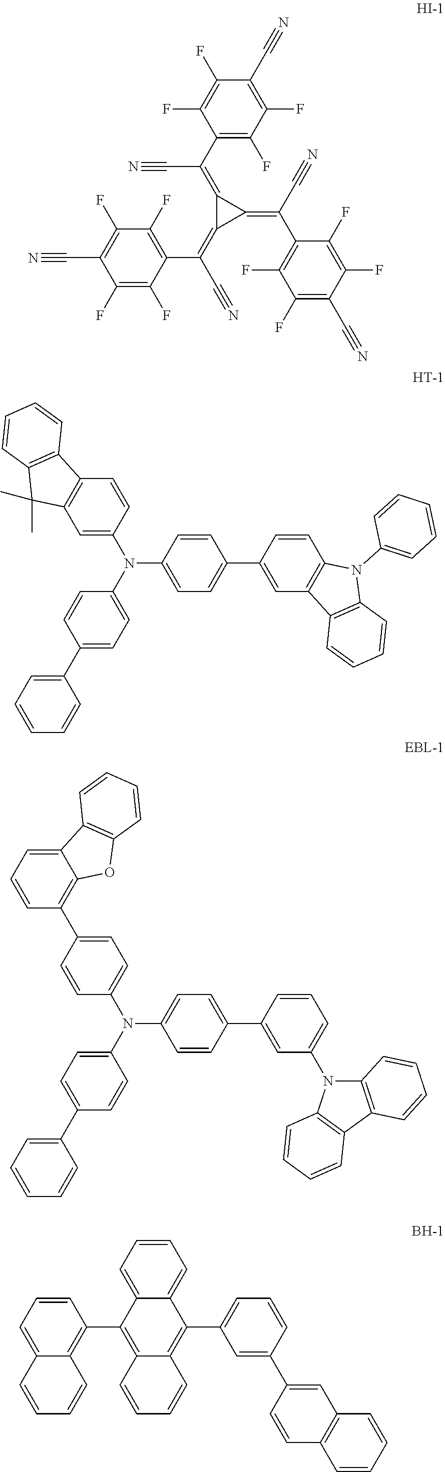

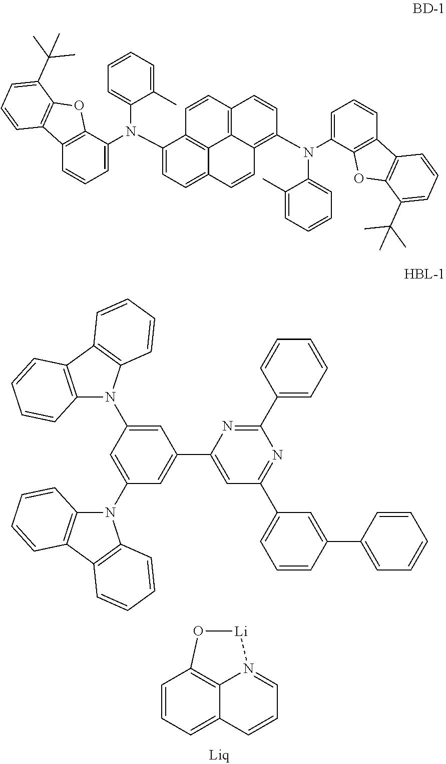

### Preparation of Organic EL Devices

Organic EL devices were prepared and evaluated as described below.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for 30 minutes. The film thickness of ITO was 130 nm.

After the glass substrate having the transparent electrode line was cleaned, the glass substrate was mounted on a substrate holder of a vacuum evaporation apparatus. Firstly, a compound HT-1 and a compound HI-1 were co-deposited on a surface of the glass substrate where the transparent electrode line was provided in a manner to cover the transparent electrode, thereby forming a 10-nm-thick hole injecting layer. The concentrations of the compound HT-1 and the compound HI-1 in the hole injecting layer were 97 mass % and 3 mass %, respectively.

Next, the compound HT-1 was vapor-deposited on the hole injecting layer to form an 80-nm-thick first hole transporting layer.

A compound EBL-1 was vapor-deposited on the first hole transporting layer to form a 5-nm-thick second hole transporting layer.

Next, a compound BH-1(host material) and a compound BD-1(dopant material) were co-deposited on the second hole transporting layer to form a 25-nm-thick emitting layer. The concentrations of the compound BH-1 and the compound BD-1 in the emitting layer were 96 mass % and 4 mass %, respectively.

Next, a compound HBL-1 was vapor-deposited on the emitting layer to form a 5-nm-thick first electron transporting layer.

The compound ET-1 and a compound Liq were co-deposited on the first electron transporting layer to form a 20-nm-thick second electron transporting layer. The ratios of the compound ET-1 and the compound Liq in the second electron transporting layer were both 50 mass %. It should be noted that Liq is an abbreviation for (8-quinolinolato)lithium.

Yb was vapor-deposited on the second electron transporting layer to form a 1-nm-thick electron injecting layer.

Metal Al was vapor-deposited on the electron injecting layer to form a 50-nm-thick cathode.

A device arrangement of the organic EL device in Example 1 is roughly shown as follows.

ITO(130)/HT-1:HI-1(10, 970/03%)/HT-1(80)/EBL-1(5)/BH-1:BD-1(25, 96%:4%)/HBL-1(5)/ET-1:Liq(20, 50%:50%)/Yb(1)/Al (50)

The numerals in parentheses represent a film thickness (unit: nm).

The numerals (97%:3%) represented by percentage in the same parentheses indicate a ratio (mass %) between the compound HT-1 and the compound HI-1 in the hole injecting layer, the numerals (96%:4%) represented by percentage in the same parentheses indicate a ratio (mass %) between the compound BH-1 and the compound BD-1 in the emitting layer, and the numerals (50%:50%) represented by percentage in the same parentheses indicate a ratio (mass %) between the compound ET-1 and the compound Liq in the second electron transporting layer.

### Examples 2 to 8

Organic EL devices of Examples 2 to 8 were each prepared as in Example 1, except that a corresponding one of the compounds described in Table 1 was used instead of the compound ET-1 included in the second electron transporting layer in Example 1.

### Comparative Example 1

An organic EL device of Comparative Example 1 was prepared as in Example 1, except that the compound described in Table 1 was used instead of the compound ET-1 included in the second electron transporting layer in Example 1.

### Evaluation of Organic EL Devices

The organic EL devices prepared in Examples 1 to 8 and Comparative Example 1 were subjected to the following evaluation. Note that, although a compound Ref-1 used in Comparative Example 1 is not the compound represented by the formula (1), the compound Ref-1 is described in the same column as “Compound represented by Formula (1)” of Example 1 for the sake of simplicity.

### Drive Voltage

The initial property of each of the organic EL devices was measured while it was driven at room temperature (25° C.) and a DC (direct current) constant-current of 50 mA/cm² in order to measure voltage (unit: V). Table 1 lists the results.

TABLE 1

| | | | | Second electron | |
|---|---|---|---|---|---|
| | | | | transporting layer | |
| | | | First electron | Compound | Voltage |
| | | | transporting | represented by | [V] @50 |
| | Emitting layer | | layer | Formula (1) | mA/cm² |
| Example 1 | BH-1 | BD-1 | HBL-1 | ET-1 | 4.65 |
| Example 2 | BH-1 | BD-1 | HBL-1 | ET-2 | 4.66 |
| Example 3 | BH-1 | BD-1 | HBL-1 | ET-3 | 4.68 |
| Example 4 | BH-1 | BD-1 | HBL-1 | ET-4 | 4.65 |
| Example 5 | BH-1 | BD-1 | HBL-1 | ET-5 | 4.54 |
| Example 6 | BH-1 | BD-1 | HBL-1 | ET-6 | 4.56 |
| Example 7 | BH-1 | BD-1 | HBL-1 | ET-8 | 4.54 |
| Example 8 | BH-1 | BD-1 | HBL-1 | ET-9 | 4.65 |
| Comparative | BH-1 | BD-1 | HBL-1 | Ref-1 | 5.00 |
| Example 1 | | | | | |

In Examples 1 to 8, where the compound represented by the formula (1) was included in the second electron transporting layer, the drive voltage was reduced compared with Comparative Example 1, where the compound represented by the formula (1) was replaced with the compound Ref-1.

### Synthesis of Compounds

### Synthesis Example 1 (Synthesis of Compound ET-1)

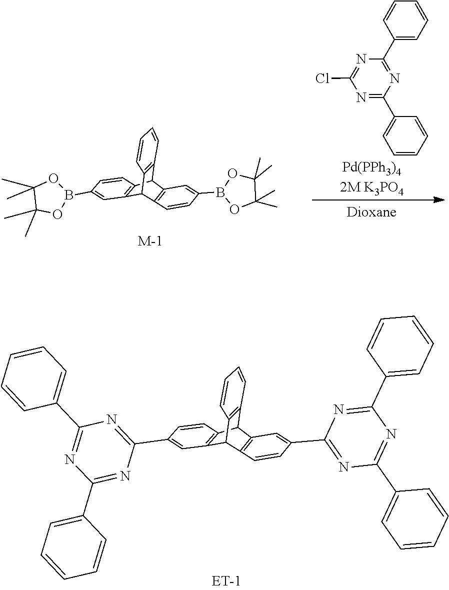

A raw material (the compound M-1: isomer mixture) (4.4 g), 2-chloro-4,6-diphenyl-1,3,5-triazine (5.12 g), Pd(PPh₃)₄ (0.603 g), 87 mL of dioxane, and 22 mL of a 2M aqueous solution of potassium phosphate were charged into a flask. Then, purging was performed with an argon gas. Subsequently, stirring was performed for 7 hours while heating was performed at 80° C. After the temperature had been reduced to room temperature (25° C.), methanol was added to the resulting reaction solution. The precipitated solid was collected by filtration and then purified by silica gel chromatography. The resulting crude product was cleaned with toluene. Hereby, 1.87 g of the compound ET-1 was prepared (yield: 30%). The results of mass spectroscopy confirmed that m/e=717 and this compound was the target substance.

Since the starting material used (compound M-1) was an isomer mixture (mixture of the compounds M-1a and M-1b), ET-1 was a mixture of ET-1a and ET-1b. Note that, in the reaction formula described in Synthesis Example 1, only the structure of the compound M-1a is described as a representative of “compound M-1”, and only the structure of the compound ET-1a is described as a representative of “compound ET-1”.

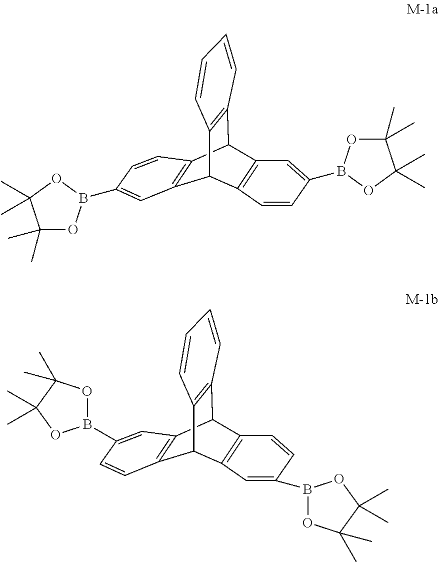

### Synthesis Example 2 (Synthesis of Compound ET-2)

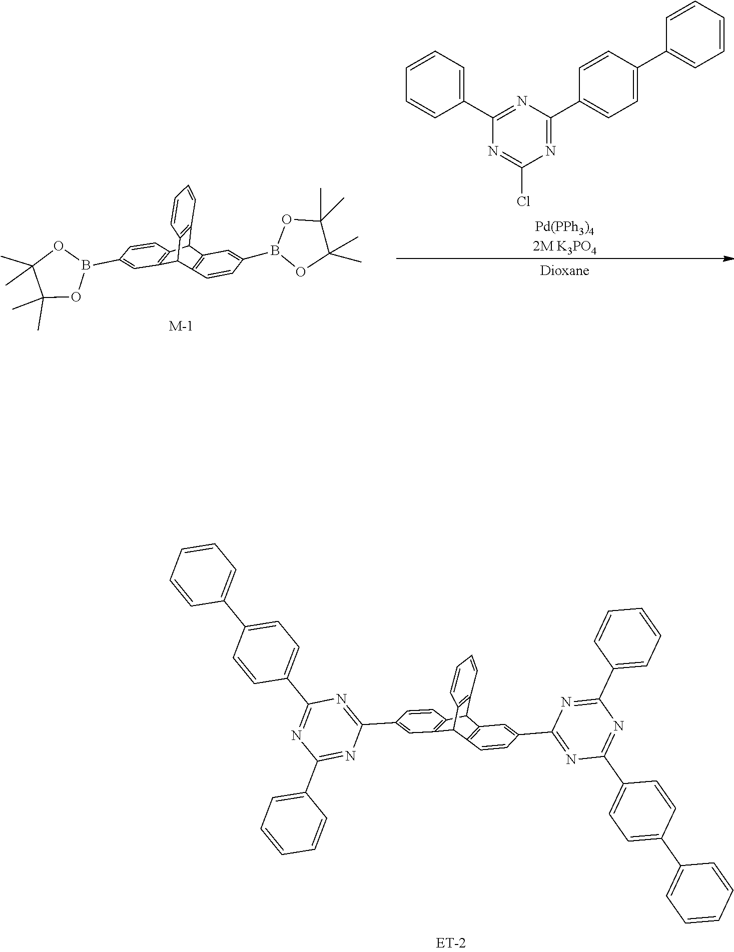

ET-2 was prepared using a raw material (compound M-1: isomer mixture) (3.0 g) and 2-([1,1′-biphenyl]-4-yl)-4-chloro-6-phenyl-1,3,5-triazine (4.5 g) under the same conditions as those described in Synthesis Example 1 as a white solid (2.0 g, yield: 39%).

The results of mass spectroscopy confirmed that m/e=869 and this compound was the target substance.

Since the starting material used (compound M-1) was an isomer mixture (mixture of the compounds M-1a and M-1b), ET-2 was a mixture of ET-2a and ET-2b. Note that, in the reaction formula described in Synthesis Example 2, only the structure of the compound M-1a is described as a representative of “compound M-1”, and only the structure of the compound ET-2a is described as a representative of “compound ET-2”.

### Synthesis Example 3 (Synthesis of Compound ET-3)

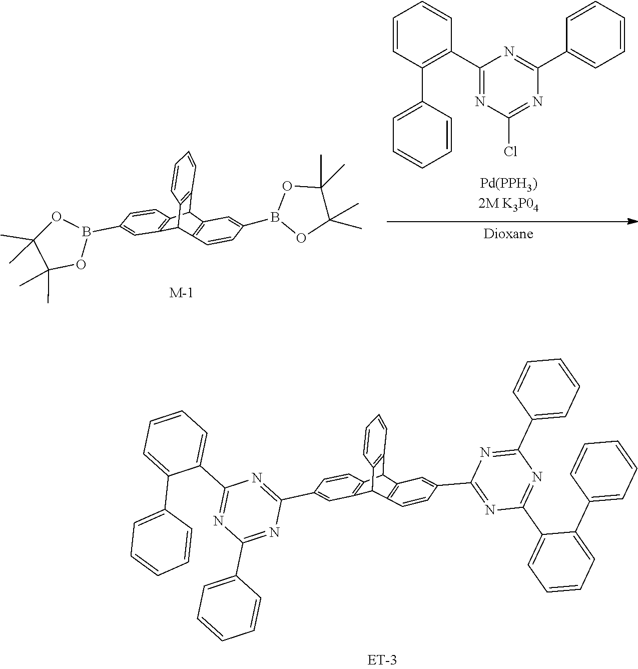

ET-3 was prepared using a raw material (compound M-1: isomer mixture) (3.0 g) and 2-([1,1′-biphenyl]-2-yl)-4-chloro-6-phenyl-1,3,5-triazine (4.5 g) under the same conditions as those described in Synthesis Example 1 as a white solid (1.9 g, yield: 36%).

The results of mass spectroscopy confirmed that m/e=869 and this compound was the target substance.

Since the starting material used (compound M-1) was an isomer mixture (mixture of the compounds M-1a and M-1b), ET-3 was a mixture of ET-3a and ET-3b. Note that, in the reaction formula described in Synthesis Example 3, only the structure of the compound M-1a is described as a representative of “compound M-1”, and only the structure of the compound ET-3a is described as a representative of “compound ET-3”.

### Synthesis Example 4 (Synthesis of Compound ET-4)

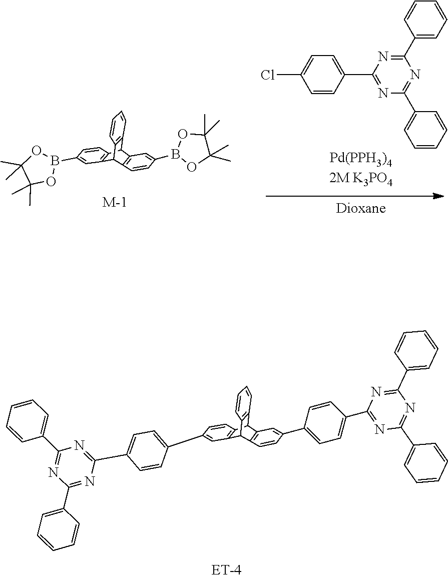

ET-4 was prepared using a raw material (compound M-1: isomer mixture) (3.0 g) and 2-(4-chlorophenyl)-4,6-diphenyl-1,3,5-triazine (4.5 g) under the same conditions as those described in Synthesis Example 1 as a white solid (1.3 g, yield: 26%).

The results of mass spectroscopy confirmed that m/e=869 and this compound was the target substance.

Since the starting material used (compound M-1) was an isomer mixture (mixture of the compounds M-1a and M-1b), ET-4 was a mixture of ET-4a and ET-4b. Note that, in the reaction formula described in Synthesis Example 4, only the structure of the compound M-1a is described as a representative of “compound M-1”, and only the structure of the compound ET-4a is described as a representative of “compound ET-4”.

### Synthesis Example 5 (Synthesis of Compound ET-5)

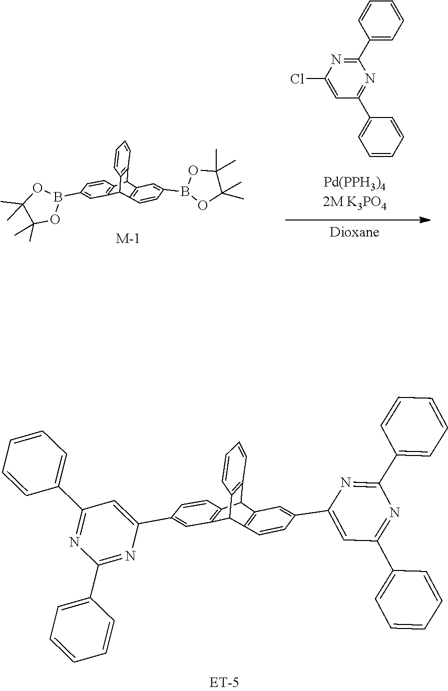

ET-5 was prepared using a raw material (compound M-1: isomer mixture) (3.0 g) and 4-chloro-2,6-diphenylpyrimidine (3.5 g) under the same conditions as those described in Synthesis Example 1 as a white solid (1.8 g, yield: 42%).

The results of mass spectroscopy confirmed that m/e=715 and this compound was the target substance.

Since the starting material used (compound M-1) was an isomer mixture (mixture of the compounds M-1a and M-1b), ET-5 was a mixture of ET-5a and ET-5b. Note that, in the reaction formula described in Synthesis Example 5, only the structure of the compound M-1a is described as a representative of “compound M-1”, and only the structure of the compound ET-5a is described as a representative of “compound ET-5”.

### Synthesis Example 6 (Synthesis of Compound ET-6)

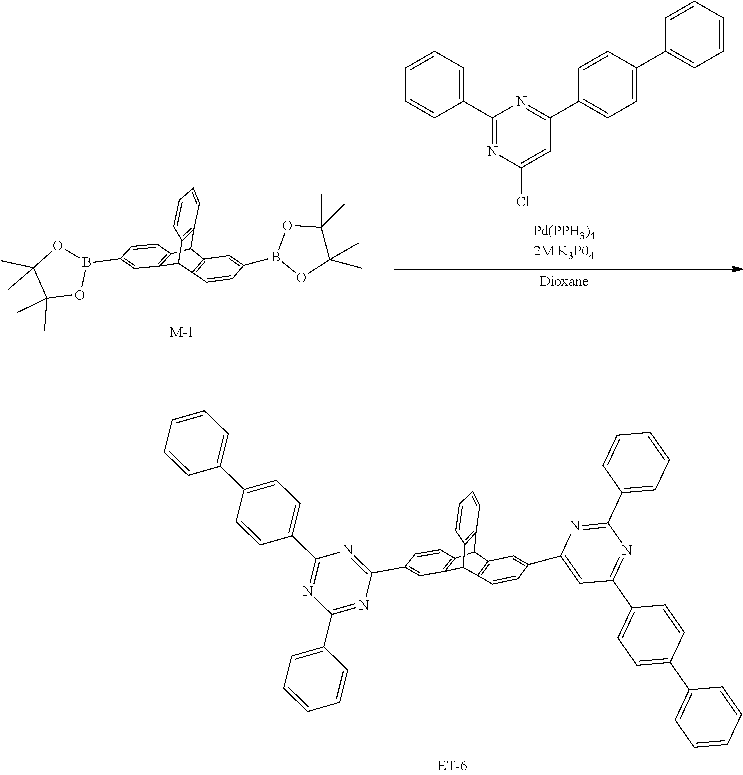

ET-6 was prepared using a raw material (compound M-1: isomer mixture) (3.0 g) and 4-([1,1′-biphenyl]-4-yl)-6-chloro-2-phenylpyrimidine (4.5 g) under the same conditions as those described in Synthesis Example 1 as a white solid (2.6 g, yield: 51%).

The results of mass spectroscopy confirmed that m/e=867 and this compound was the target substance.

Since the starting material used (compound M-1) was an isomer mixture (mixture of the compounds M-1a and M-1b), ET-6 was a mixture of ET-6a and ET-6b. Note that, in the reaction formula described in Synthesis Example 6, only the structure of the compound M-1a is described as a representative of “compound M-1”, and only the structure of the compound ET-6a is described as a representative of “compound ET-6”.

### Synthesis Example 7 (Synthesis of Compound ET-7)

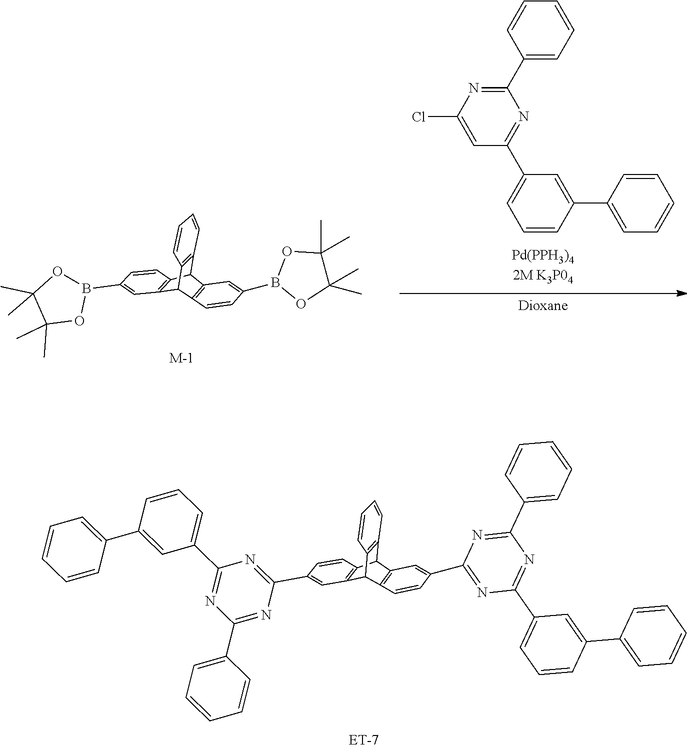

ET-7 was prepared using a raw material (compound M-1: isomer mixture) (3.0 g) and 4-([1,1′-biphenyl]-3-yl)-6-chloro-2-phenylpyrimidine (4.5 g) under the same conditions as those described in Synthesis Example 1 as a white solid (1.6 g, yield: 32%).

The results of mass spectroscopy confirmed that m/e=867 and this compound was the target substance.

Since the starting material used (compound M-1) was an isomer mixture (mixture of the compounds M-1a and M-1b), ET-7 was a mixture of ET-7a and ET-7b. Note that, in the reaction formula described in Synthesis Example 7, only the structure of the compound M-1a is described as a representative of “compound M-1”, and only the structure of the compound ET-7a is described as a representative of “compound ET-7”.

### Synthesis Example 8 (Synthesis of Compound ET-8)

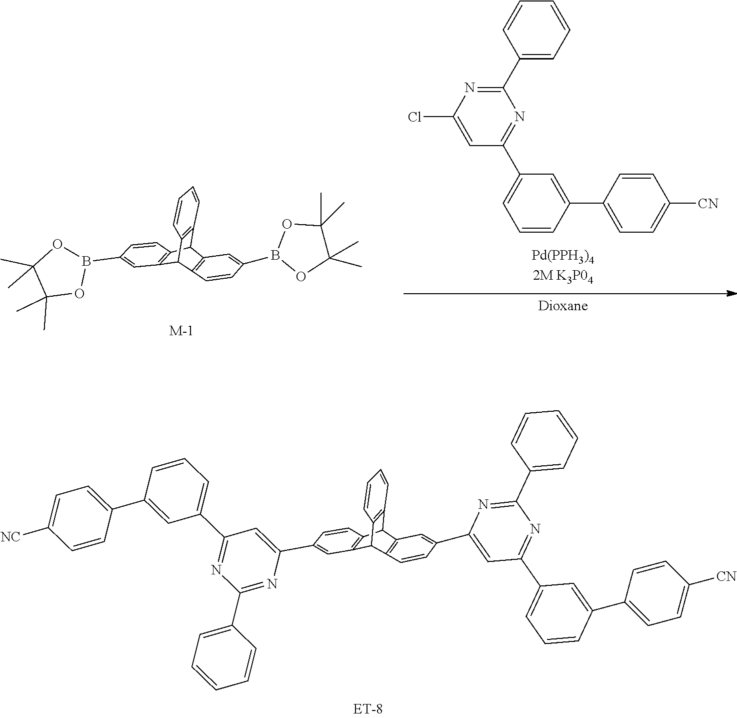

ET-8 was prepared using a raw material (compound M-1: isomer mixture) (3.0 g) and 3′-(6-chloro-2-phenylpyrimidin-4-yl)-[1,1′-biphenyl]-4-carbonitrile (4.8 g) under the same conditions as those described in Synthesis Example 1 as a white solid (1.5 g, yield: 28%).

The results of mass spectroscopy confirmed that m/e=917 and this compound was the target substance.

Since the starting material used (compound M-1) was an isomer mixture (mixture of the compounds M-1a and M-1b), ET-8 was a mixture of ET-8a and ET-8b. Note that, in the reaction formula described in Synthesis Example 8, only the structure of the compound M-1a is described as a representative of “compound M-1”, and only the structure of the compound ET-8a is described as a representative of “compound ET-8”.

### Synthesis Example 9 (Compound ET-9)

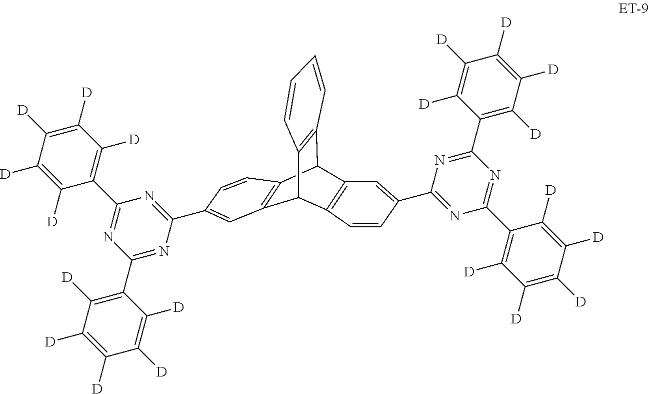 [SMILES: [2H]c1c([2H])c([2H])c(-c2nc(-c3ccc4c(c3)C3c5ccccc5C4c4cc(-c5nc(-c6c([2H])c([2H])c([2H])c([2H])c6[2H])nc(-c6c([2H])c([2H])c([2H])c([2H])c6[2H])n5)ccc43)nc(-c3c([2H])c([2H])c([2H])c([2H])c3[2H])n2)c([2H])c1[2H]]

ET-9 was synthesized by the same synthesis scheme as in Synthesis Example 1, except that 2-chloro-4,6-di(phenyl-2,3,4,5,6-d5)-1,3,5-triazine was used instead of 2-chloro-4,6-diphenyl-1,3,5-triazine.

The results of mass spectroscopy confirmed that m/e=736 and this compound was the target substance.

Since the starting material used (compound M-1) was an isomer mixture (mixture of the compounds M-1a and M-1b), ET-9 was a mixture of ET-9a and ET-9b. Note that, in the reaction formula described in Synthesis Example 9, only the structure of the compound ET-9a is described as a representative of “compound ET-9”.

### Comparative Synthesis Example 1 (Synthesis of Compound Ref-1)

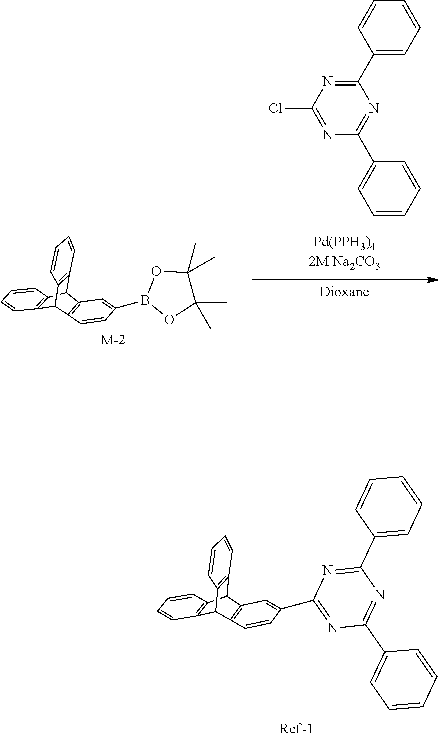

Into a flask, 2-chloro-4,6-diphenyl-1,3,5-triazine (3.01 g), the compound M-2 (2.02 g), Pd(PPh₃)₄ (0.436 g), 76 mL of dioxane, and 9.4 mL of a 2M aqueous solution of sodium carbonate were charged. Then, purging was performed with an argon gas. Subsequently, heating and stirring were performed for 8 hours while refluxing was performed. After the temperature had been reduced to room temperature (25° C.), the resulting solution was concentrated. Subsequently, methanol was added to the solution. The precipitated solid was collected by filtration and then purified by silica gel chromatography. The resulting crude product was recrystallized using toluene. Hereby, 2.86 g of a compound Ref-1 was prepared (yield: 78%). The results of mass spectroscopy confirmed that m/e=486 and this compound was the target substance.

What is claimed is:

1. A compound represented by a formula (1) below, 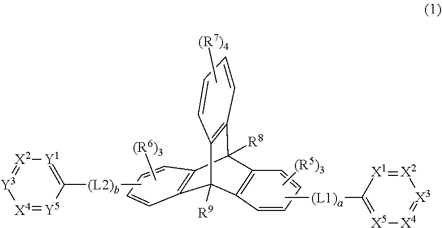
in the formula (1),
X¹ to X⁵ each independently represent a nitrogen atom or CR¹⁰,
two or more of X¹ to X⁵ are each a nitrogen atom,
at least one combination of adjacent two or more of a plurality of R¹⁰ are bonded to each other to form a substituted or unsubstituted monocyclic ring, are bonded to each other to form a substituted or unsubstituted fused ring, or are not bonded to each other,
Y¹ to Y⁵ each independently represent a nitrogen atom or CR²⁰,
one or more of Y¹ to Y⁵ are each a nitrogen atom,
at least one combination of adjacent two or more of a plurality of R²⁰ are bonded to each other to form a substituted or unsubstituted monocyclic ring, are bonded to each other to form a substituted or unsubstituted fused ring, or are not bonded to each other,
at least one combination of adjacent two or more of a plurality of R⁵ are bonded to each other to form a substituted or unsubstituted monocyclic ring, are bonded to each other to form a substituted or unsubstituted fused ring, or are not bonded to each other,
at least one combination of adjacent two or more of a plurality of R⁶ are bonded to each other to form a substituted or unsubstituted monocyclic ring, are bonded to each other to form a substituted or unsubstituted fused ring, or are not bonded to each other,
at least one combination of adjacent two or more of a plurality of R⁷ are bonded to each other to form a substituted or unsubstituted monocyclic ring, are bonded to each other to form a substituted or unsubstituted fused ring, or are not bonded to each other,
R¹⁰, R²⁰, and R⁵ to R⁷ forming neither the substituted or unsubstituted monocyclic ring nor the substituted or unsubstituted fused ring, and R⁸ and R⁹ each independently represent a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms,
when a plurality of R¹⁰ are present, the plurality of R¹⁰ are mutually the same or different,
when a plurality of R²⁰ are present, the plurality of R²⁰ are mutually the same or different,
the plurality of R⁵ are mutually the same or different,
the plurality of R⁶ are mutually the same or different,
the plurality of R⁷ are mutually the same or different,
a represents 0, 1, 2, or 3,
when a is 2 or 3, a plurality of L1 are mutually the same or different,
b represents 0, 1, 2, or 3,
when b is 2 or 3, a plurality of L2 are mutually the same or different,
when a and b are each independently 1, 2, or 3, L1 and L2 each independently represent a single bond, a substituted or unsubstituted arylene group having 6 to 50 ring carbon atoms, or a substituted or unsubstituted divalent heterocyclic group having 5 to 50 ring atoms,
when only one of Y¹ to Y⁵ is a nitrogen atom, L2 is not a single bond, or any of Y¹ to Y⁵ other than a nitrogen atom is not CH,
in the compound represented by the formula (1), R₉₀₁ to R₉₀₇ each independently represent a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms,
when two or more R₉₀₁ are present, the two or more R₉₀₁ are mutually the same or different,
when two or more R₉₀₂ are present, the two or more R₉₀₂ are mutually the same or different,
when two or more R₉₀₃ are present, the two or more R₉₀₃ are mutually the same or different,
when two or more R₉₀₄ are present, the two or more R₉₀₄ are mutually the same or different,
when two or more R₉₀₅ are present, the two or more R₉₀₅ are mutually the same or different,
when two or more R₉₀₆ are present, the two or more R₉₀₆ are mutually the same or different, and
when two or more R₉₀₇ are present, the two or more R₉₀₇ are mutually the same or different.

2. The compound according to claim 1, wherein
at least one combination of adjacent two or more of the plurality of R¹⁰ are not bonded to each other,
at least one combination of adjacent two or more of the plurality of R²⁰ are not bonded to each other,
at least one combination of adjacent two or more of the plurality of R⁵ are not bonded to each other,
at least one combination of adjacent two or more of the plurality of R⁶ are not bonded to each other, and
at least one combination of adjacent two or more of the plurality of R⁷ are not bonded to each other.

3. The compound according to claim 1, wherein
in the formula (1), among partial structures represented by formulae (1A), (1B), and (1C) below, the partial structures represented by the formulae (1A) and (1B) are different from each other, 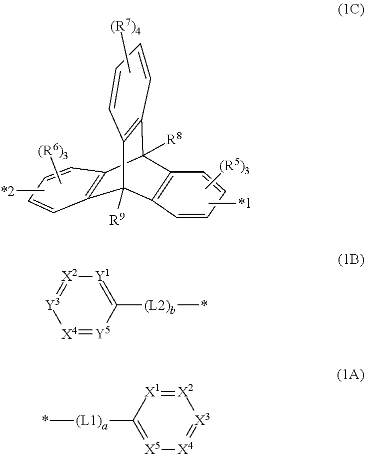
in the formula (1C), R⁵ to R⁹ each independently represent the same as R⁵ to R⁹ in the formula (1),
in the formula (1A), X¹ to X⁵, L1, and a each independently represent the same as X¹ to X⁵, L1, and a in the formula (1), and * represents a bonding position to *1 in the partial structure represented by the formula (1C) in the formula (1), and
in the formula (1B), Y¹ to Y⁵, L2, and b each independently represent the same as Y¹ to Y⁵, L2, and b in the formula (1), and * represents a bonding position to *2 in the partial structure represented by the formula (1C) in the formula (1).

4. The compound according to claim 1, wherein
in the formula (1), partial structures represented by formulae (1A) and (1B) below are the same as each other, 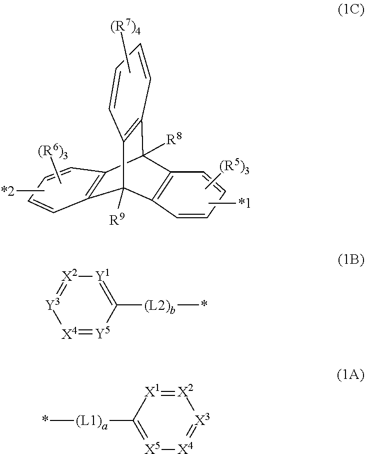
in the formula (1C), R⁵ to R⁹ each independently represent the same as R⁵ to R⁹ in the formula (1),
in the formula (1A), X¹ to X⁵, L1, and a each independently represent the same as X¹ to X⁵, L1, and a in the formula (1), and * represents a bonding position to *1 in the partial structure represented by the formula (1C) in the formula (1), and
in the formula (1B), Y¹ to Y⁵, L2, and b each independently represent the same as Y¹ to Y⁵, L2, and b in the formula (1), and * represents a bonding position to *2 in the partial structure represented by the formula (1C) in the formula (1).

5. The compound according to claim 3, wherein
in the formula (1A), two or three of X¹ to X⁵ are each a nitrogen atom, and
in the formula (1B), one, two, or three of Y¹ to Y⁵ are each a nitrogen atom.

6. The compound according to claim 3, wherein
the partial structure represented by the formula (1A) is represented by any of formulae (1A-1) to (1A-3) below, and
the partial structure represented by the formula (1B) is represented by any of formulae (1B-1) to (1B-6) below, 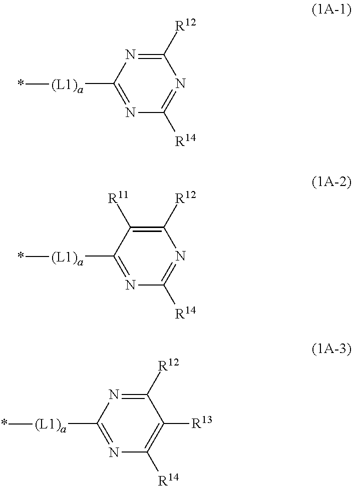
in the formulae (1A-1) to (1A-3), L1 and a each independently represent the same as L1 and a in the formula (1), and R¹¹, R¹², R¹³, and R¹⁴ each independently represent the same as R¹⁰ in the formula (1), 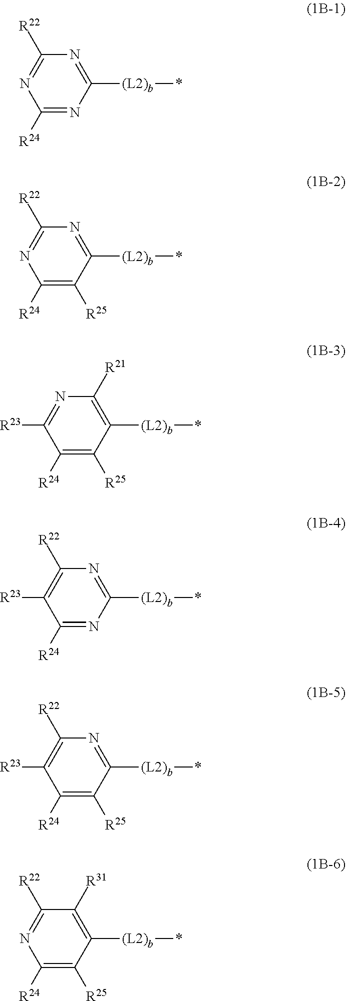
in the formulae (1B-1) to (1B-6), L2 and b each independently represent the same as L2 and b in the formula (1), and R²¹ to R²⁵ each independently represent the same as R²⁰ in the formula (1).

7. The compound according to claim 6, wherein
when the partial structure represented by the formula (1A) is represented by the formula (1A-1), the partial structure represented by the formula (1B) is represented by the formula (1B-1),
when the partial structure represented by the formula (1A) is represented by the formula (1A-2), the partial structure represented by the formula (1B) is represented by the formula (1B-2), and
when the partial structure represented by the formula (1A) is represented by the formula (1A-3), the partial structure represented by the formula (1B) is represented by the formula (1B-4).

8. The compound according to claim 1, wherein
the compound represented by the formula (1) is a compound represented by a formula (10) below, 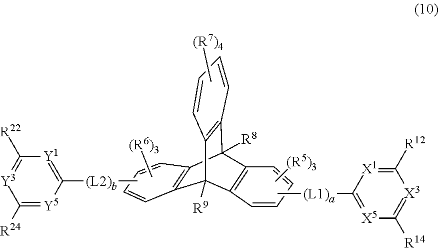
in the formula (10), X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and
R¹² and R¹⁴ each independently represent the same as R¹⁰ in the formula (1), and R²² and R²⁴ each independently represent the same as R²⁰ in the formula (1).

9. The compound according to claim 1, wherein
the compound represented by the formula (1) is a compound represented by a formula (10-1a) below or an enantiomer of the compound represented by the formula (10-1a), 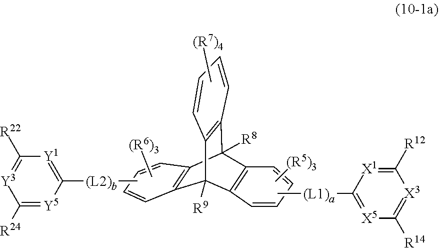
in the formula (10-1a), X¹, X³, X⁵, Y¹, Y³, Y⁵, R⁵ to R⁹, L1, L2, a, and b each independently represent the same as X¹, X³, X⁵, Y¹, Y³, Y⁵, R⁵ to R⁹, L1, L2, a, and b in the formula (1), and
R¹² and R¹⁴ each independently represent the same as R¹⁰ in the formula (1), and R²² and R²⁴ each independently represent the same as R²⁰ in the formula (1).

10. The compound according to claim 1, wherein
L1 and L2 each independently represent a single bond, a substituted or unsubstituted arylene group having 6 to 30 ring carbon atoms, or a substituted or unsubstituted divalent heterocyclic group having 5 to 30 ring atoms.

11. The compound according to claim 1, wherein
L1 and L2 each independently represent a single bond, a substituted or unsubstituted phenylene group, a substituted or unsubstituted biphenylene group, a substituted or unsubstituted terphenylene group, a substituted or unsubstituted naphthylene group, a substituted or unsubstituted phenanthrylene group, a substituted or unsubstituted fluorenylene group, a substituted or unsubstituted dibenzofuranylene group, a substituted or unsubstituted dibenzothienylene group, a substituted or unsubstituted pyridylene group, or a substituted or unsubstituted quinolylene group.

12. The compound according to claim 1, wherein
a is 0 or 1, and b is 0 or 1.

13. The compound according to claim 1, wherein
R¹⁰, R²⁰, and R⁵ to R⁹ each independently represent a hydrogen atom, a halogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —N(R₉₀₆)(R₉₀₇), a substituted or unsubstituted aryl group having 6 to 30 ring carbon atoms, or a substituted or unsubstituted heterocyclic group having 5 to 30 ring atoms.

14. The compound according to claim 1, wherein
R¹⁰, R²⁰and R⁵ to R⁹ each independently represent a hydrogen atom, a halogen atom, a cyano group, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms.

15. The compound according to claim 1, wherein
R¹⁰, R²⁰, and R⁵ to R⁹ each independently represent a hydrogen atom, or a group represented by any of formulae (A1) to (A31) below, 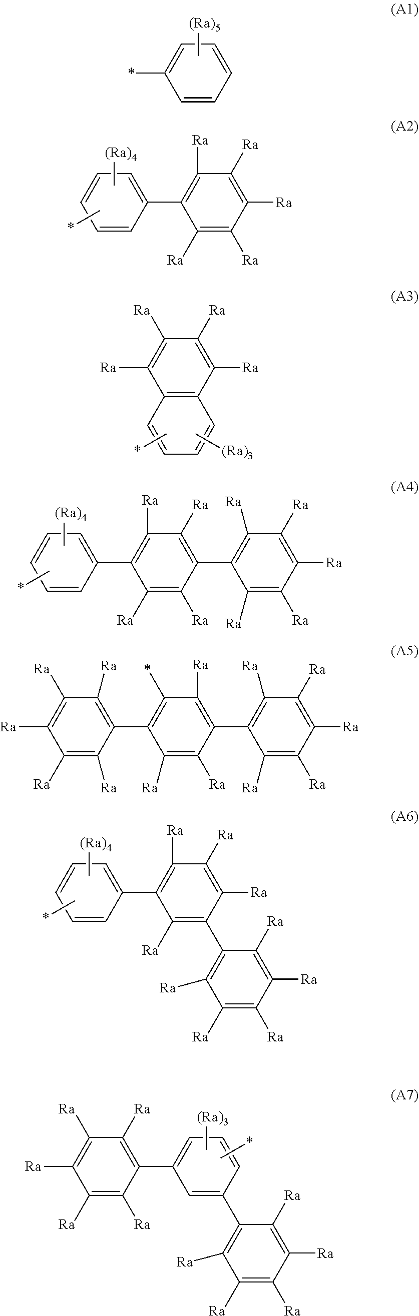 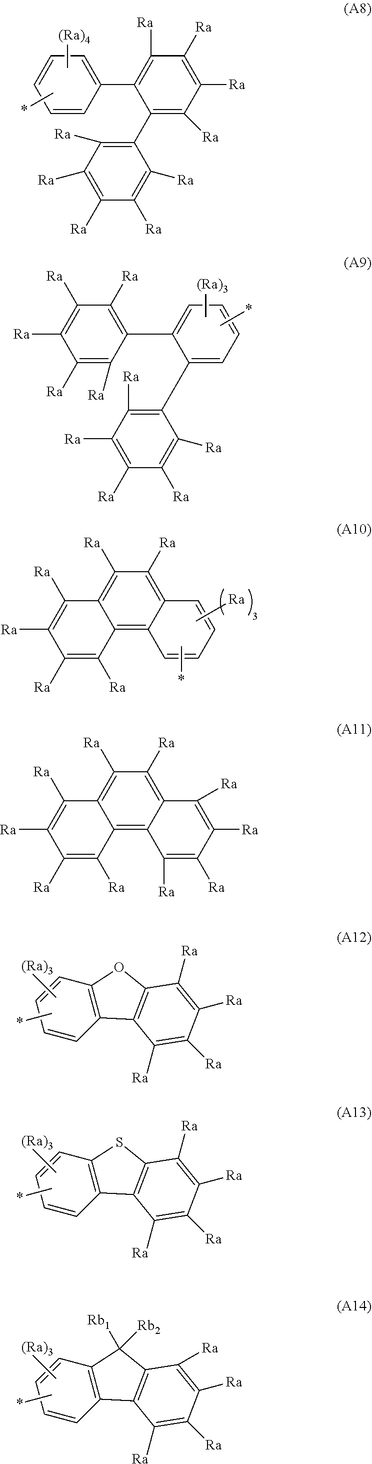 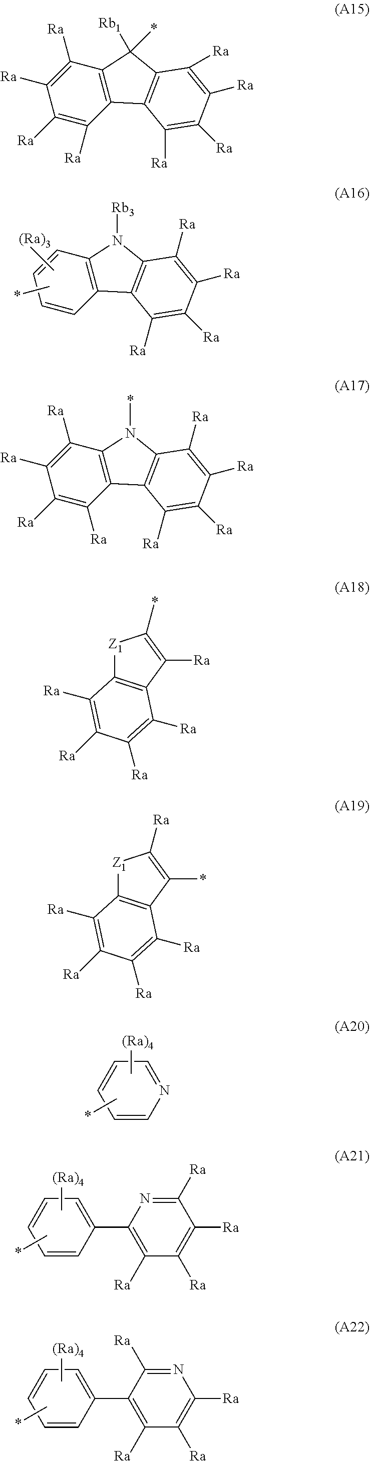 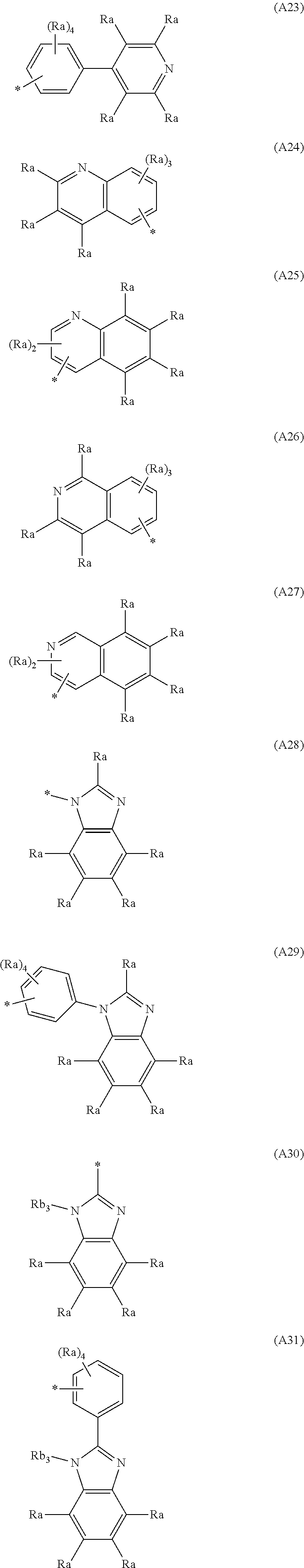
in the formulae (A1) to (A31),
Z₁ represents an oxygen atom, a sulfur atom, or NRb₃,
at least one combination of adjacent two or more of a plurality of Ra are bonded to each other to form a substituted or unsubstituted monocyclic ring, are bonded to each other to form a substituted or unsubstituted fused ring, or are not bonded to each other,
a pair of Rb₁ and Rb₂ are bonded to each other to form a substituted or unsubstituted monocyclic ring, are bonded to each other to form a substituted or unsubstituted fused ring, or are not bonded to each other,
Rb₁ and Rb₂ forming neither the substituted or unsubstituted monocyclic ring nor the substituted or unsubstituted fused ring, and Rb₃ each independently represent a hydrogen atom, a substituted or unsubstituted alkyl group having 1 to 30 carbon atoms, a substituted or unsubstituted cycloalkyl group having 3 to 30 ring carbon atoms, a substituted or unsubstituted aryl group having 6 to 30 ring carbon atoms, or a substituted or unsubstituted heterocyclic group having 5 to 30 ring atoms,
Ra forming neither the substituted or unsubstituted monocyclic ring nor the substituted or unsubstituted fused ring each independently represent a hydrogen atom, a halogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —N(R₉₀₆)(R₉₀₇), a substituted or unsubstituted aryl group having 6 to 30 ring carbon atoms, or a substituted or unsubstituted heterocyclic group having 5 to 30 ring atoms,
R₉₀₁ to R₉₀₄, R₉₀₆, and R₉₀₇ each independently represent the same as R₉₀₁ to R₉₀₄, R₉₀₆, and R₉₀₇ in the formula (1),
a plurality of Ra are mutually the same or different,
when a plurality of Z₁ are present, the plurality of Z₁ are mutually the same or different,
when a plurality of Rb₁ are present, the plurality of Rb₁ are mutually the same or different,
when a plurality of Rb₂ are present, the plurality of Rb₂ are mutually the same or different, and
when a plurality of Rb₃ are present, the plurality of Rb₃ are mutually the same or different.

16. The compound according to claim 1, wherein
R⁵ to R⁹ each represent a hydrogen atom, and R¹⁰ and R²⁰ each independently represent a hydrogen atom, a halogen atom, a cyano group, a substituted or unsubstituted alkyl group having 1 to 18 carbon atoms, a substituted or unsubstituted cycloalkyl group having 3 to 18 ring carbon atoms, a substituted or unsubstituted aryl group having 6 to 18 ring carbon atoms, or a substituted or unsubstituted heterocyclic group having 5 to 18 ring atoms.

17. A mixture comprising:
the compound according to claim 1 as a first compound; and
a second compound that is an enantiomer of the first compound.

18. A material for organic electroluminescence device comprising the compound according to claim 1.

19. A material for organic electroluminescence device comprising the mixture according to claim 17.

20. An organic electroluminescence device comprising:
a cathode;
an anode; and
one or more organic layers interposed between the cathode and the anode, wherein
at least one of the organic layers comprises the compound according to claim 1 as a first compound.

21. The organic electroluminescence device according to claim 20, wherein
the organic layers comprise: an emitting layer interposed between the cathode and the anode; and an electron transporting layer interposed between the cathode and the emitting layer, and
the electron transporting layer comprises the first compound.

22. The organic electroluminescence device according to claim 21 wherein,
the electron transporting layer comprises: a first electron transporting layer interposed between the cathode and the emitting layer, and a second electron transporting layer interposed between the first electron transporting layer and the cathode, and
the second electron transporting layer comprises the first compound.

23. The organic electroluminescence device according to claim 21, wherein
the organic layers comprise a hole transporting layer interposed between the anode and the emitting layer.

24. The organic electroluminescence device according to claim 20, wherein
at least one of the organic layers comprises the first compound and a second compound that is an enantiomer of the first compound.

25. An electronic device comprising the organic electroluminescence device according to claim 20.

26. The compound according to claim 4, wherein
in the formula (1A), two or three of X¹ to X⁵ are each a nitrogen atom, and
in the formula (1B), two or three of Y¹ to Y⁵ are each a nitrogen atom.

27. The compound according to claim 4, wherein
when the partial structure represented by the formula (1A) is represented by a formula (1A-1) below, the partial structure represented by the formula (1B) is represented by a formula (1B-1) below,
when the partial structure represented by the formula (1A) is represented by a formula (1A-2) below, the partial structure represented by the formula (1B) is represented by a formula (1B-2) below; and
when the partial structure represented by the formula (1A) is represented by a formula (1A-3) below, the partial structure represented by the formula (1B) is represented by a formula (1B-4) below, 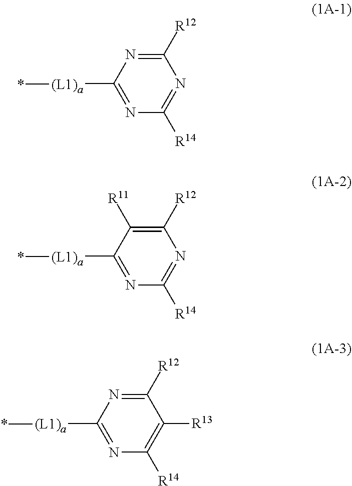
in the formulae (1A-1) to (1A-3), L1 and a each independently represent the same as L1 and a in the formula (1), and R¹¹, R¹², R¹³, and R¹⁴ each independently represent the same as R¹⁰ in the formula (1),
in the formulae (1B-1), (1B-2), and (1B-4), L2 and b each independently represent the same as L2 and b in the formula (1), and R²² to R²⁵ each independently represent the same as R²⁰ in the formula (1).
